(19) United States
(12) Patent Application Publication
(10) Pub. No.: US 2025/0171431 A1
(43) Pub. Date: May 29, 2025

(54) HETEROCYCLIC INHIBITORS OF IGF-1R FOR TREATMENT OF DISEASE

(71) Applicant: HORIZON THERAPEUTICS IRELAND DAC, DUBLIN (IE)

(72) Inventors: ASHLEY FENWICK, NAPERVILLE, IL (US); MARTA E. WENZLER, CHICAGO, IL (US); DENIS BILLEN, BARRINGTON, IL (US); YEEMAN K. RAMTOHUL, MONTREAL (CA); WILLIAM BROWN, MONTREAL (CA); NARESH VEMULA, DORION (CA); NICHOLAS WILLET MICHEL, Montreal (CA); THOMAS JOHNSON, SAINT-LAURENT (CA); JACOB SOLEY, SAINT LAURENT (CA); GEOFFREY S. SINCLAIR, MONTREAL (CA); KEN LOON CHOO, MONTREAL (CA); Yuki Maekawa, MONTREAL (CA); TABINDA AHMAD, MONTREAL (CA)

(21) Appl. No.: 18/793,698

(22) Filed: Aug. 2, 2024

Related U.S. Application Data
(60) Provisional application No. 63/517,285, filed on Aug. 2, 2023.

(51) Int. Cl. C07D 417/14; A61K 31/506; A61K 31/5377; A61K 31/538; A61K 31/5386; A61K 31/541; A61K 31/55; C07D 401/12; C07D 401/14; C07D 403/12; C07D 413/14; C07D 471/04; C07D 471/08; C07D 491/044; C07D 491/056; C07D 491/08; C07D 491/107; C07D 491/113; C07D 495/04

(52) U.S. Cl. CPC C07D 417/14; A61K 31/506; A61K 31/5377; A61K 31/538; A61K 31/5386; A61K 31/541; A61K 31/55; C07D 401/12; C07D 401/14; C07D 403/12; C07D 413/14; C07D 471/04; C07D 471/08; C07D 491/044; C07D 491/056; C07D 491/08; C07D 491/107; C07D 491/113; C07D 495/04

(57) ABSTRACT

The present invention relates to polycyclic heterocyclic compounds and methods which may be useful as inhibitors of IGF-1R for the treatment or prevention of thyroid eye disease (TED), also known as thyroid-associated ophthalmopathy (TAO), or Graves' ophthalmopathy or orbitopathy (GO).

### CROSS-REFERENCE TO RELATED APPLICATIONS

This application claims the benefit of priority to U.S. Provisional Patent Application No. 63/517,285, filed Aug. 2, 2023, which is hereby incorporated by reference in its entirety as if fully set forth herein.

### BACKGROUND OF THE INVENTION

Disclosed herein are novel polycyclic heterocyclic compounds and compositions and their use for the treatment of diseases or disorders, including but not limited to thyroid eye disease (TED). Methods of inhibition of insulin-like growth factor 1 receptor (IGF-1R) activity in a mammal are also provided for the treatment diseases or disorders such as thyroid eye disease (TED), also known as thyroid-associated ophthalmopathy (TAO), or Graves' ophthalmopathy or orbitopathy (GO).

TED is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos.

TED is commonly considered to be the autoimmune orbital manifestation of Graves' Disease (GD). However, only approximately 30% of patients with Graves' hyperthyroidism manifest clinically relevant ocular pathology indicating there is mechanistic heterogeneity and differentiation between the conditions. Although the molecular mechanisms underlying TED remain unclear, it is accepted that the generation of autoantibodies that act as agonists on the thyroid-stimulating hormone receptor (TSHR) is responsible for Graves' hyperthyroidism. Pathogenic overstimulation of TSHR, leads to overproduction of thyroid hormones (T3 and T4) and accelerated metabolism of many tissues.

Antibodies that activate the insulin-like growth factor 1 receptor (IGF-1R) have also been detected and implicated in active TED. Without being bound to any theory, it is believed that TSHR and IGF-1R form a physical and functional complex in orbital fibroblasts, and that blocking IGF-1R appears to attenuate both IGF-1 and TSH-dependent signaling. It has been suggested that blocking IGF-1R activity might reduce both TSHR- and IGF-1-dependent signaling and therefore interrupt the pathological activities of autoantibodies acting as agonists on either receptor.

IGF-1R is a widely expressed heterotetrameric protein involved in the regulation of proliferation and metabolic function of many cell types. It is a tyrosine kinase receptor comprising two subunits. IGF-1Rα contains a ligand-binding domain while IGF-1Rβ is involved in signaling and contains tyrosine phosphorylation sites.

Management of hyperthyroidism due to Graves' disease is imperfect because therapies targeting the specific underlying pathogenic autoimmune mechanisms of the disease are lacking. Even more complex is the treatment of moderate-to-severe active TED. Although recent years have witnessed a better understanding of its pathogenesis, TED remains a therapeutic challenge and dilemma. Intravenous glucocorticoids (ivGCs) and oral glucocorticoids are used to treat patients with moderate-to-severe active TED, but results are seldom satisfactory. Partial responses are frequent and relapses (rebound) after drug withdrawal are not uncommon. Adverse events do occur and many patients eventually require rehabilitative surgery conducted when their condition has transitioned to inactive TED. Teprotumumab, a fully human IGF-1R inhibitory monoclonal antibody, is the only therapeutic specifically approved for patients with active, moderate-to-severe TED.

There is a need for novel therapeutics, specifically small molecules, that inhibit IGF-1R activity. Disclosed herein are novel IGF-1R inhibitory compounds, methods of synthesizing and using the compounds including methods for the treatment of IGF-1R-mediated diseases or disorders in a patient by administering the compounds.

### BRIEF SUMMARY OF THE INVENTION

Compounds disclosed herein inhibit IGF-1R activity.

In one aspect, described herein is a compound of Formula (I):

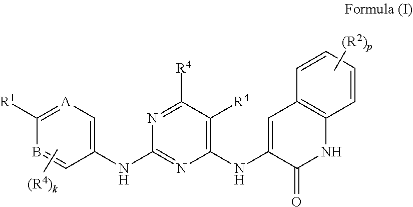

or a pharmaceutically acceptable salt or solvate thereof, wherein:
   R¹ is unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —OR²⁰, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   or R¹ is

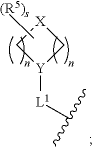

each n is independently 1, 2, or 3;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
Y is CH; and L¹ is —O—, —S—, —N(R²¹)—, -(unsubstituted or substituted C₁-C₆alkyl)-O—, -(unsubstituted or substituted C₁-C₆alkyl)-S—, or -(unsubstituted or substituted C₁-C₆alkyl)-N(R²¹)—;
or Y is N; and L¹ is -(unsubstituted or substituted C₁-C₆alkyl)-O—, -(unsubstituted or substituted C₁-C₆alkyl)-S—, or -(unsubstituted or substituted C₁-C₆alkyl)-N(R²¹)—;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
or both R⁸ are taken together with the carbon to which they are attached to form a unsubstituted or substituted C₃-C₈cycloalkyl, unsubstituted or substituted C₃-C₈heterocycloalkyl;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
B is C(R³) or N;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R¹, —CO₂R²¹, —N(R²¹)₂, —SR²⁰, —S(═O)R²⁰, or —SO₂R²⁰;
or R¹ and R³ are taken together with the intervening carbon atoms connecting R¹ to R³ to form a ring A that is monocyclic C₃-C₈cycloalkyl, monocyclic C₃-C₈heterocycloalkyl, bicyclic C₅-C₁₀cycloalkyl, or bicyclic C₅-C₁₀heterocycloalkyl, wherein ring A is unsubstituted or substituted with 1 or 2 R¹⁴, or 1 or 2 R¹⁵;
or R¹ and R³ are taken together with the intervening carbon atoms connecting R¹ to R³ to form a ring A that is monocyclic C₃-C₈cycloalkyl, bicyclic C₅-C₁₀cycloalkyl, monocyclic C₃-C₈heterocycloalkyl, or bicyclic C₅-C₁₀heterocycloalkyl, wherein ring A is unsubstituted or substituted with one or more groups independently selected from the group consisting of R¹⁴, R¹⁵, R¹⁶, and R¹⁷;
each R¹⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, substituted or unsubstituted C₁-C₆alkyl-N(R²¹)₂, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R¹⁵ is -substituted or unsubstituted C₁-C₆alkyl, -substituted or unsubstituted C₁-C₆alkyl-N(R²¹)₂, —C(═O)-substituted or unsubstituted C₁-C₆alkyl-N(R²¹)₂, -substituted or unsubstituted C₁-C₆alkyl-carbocycle, or heterocycle;
each X^{a} is independently —C(R¹⁶)₂—, —N(R¹⁷)—, —O—, or —S—;
each R¹⁶ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹⁷ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —C(═O)R²¹, —CO₂R²¹, or —C(═O)N(R²¹)₂;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
p is 0, 1, 2, 3, or 4;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In another aspect described herein is a compound of Formula (II):

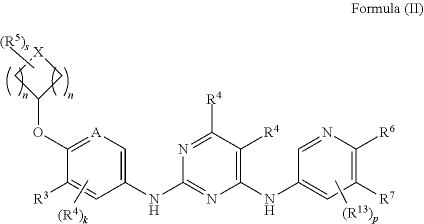

or a pharmaceutically acceptable salt or solvate thereof, wherein:
   each n is independently 1, 2, or 3;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R², —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   or both R⁸ are taken together with the carbon to which they are attached to form a unsubstituted or substituted C₃-C₈cycloalkyl, unsubstituted or substituted C₃-C₈heterocycloalkyl;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
   or R⁶ and R⁷ are taken together with the intervening carbon atoms connecting R⁶ to R⁷ to form

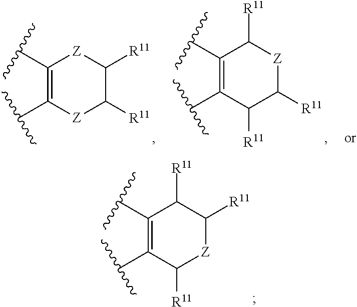

each Z is independently —C(R¹¹)₂, —N(R¹²)—, —O—, —S—, or —SO₂—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
each R¹³ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
p is 0, 1, 2, 3, or 4
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted heterocycle containing 1 or 2 N atoms, 0 or 1 O atoms, and 0 or 1 S, S(O), or S(O)₂.

In another aspect described herein is a compound of Formula (IIIA1):

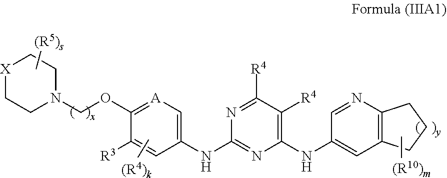

or a pharmaceutically acceptable salt or solvate thereof, wherein:
   x is 1, 2, 3, 4, 5, or 6;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R²¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   y is 1, 2, or 3;
   each R¹⁰ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —CO₂R²¹, —C(═O)N(R²¹)₂, —N(R²¹)₂, —NR²¹C(═O)R²¹, —SR²⁰, —S(═O)R²⁰, —SO₂R²⁰, or —SO₂N(R²¹);
   m is 0, 1, 2, 3, or 4;
   wherein two R¹⁰ can be optionally connected by L;
   L is C₁-C₆ alkyl, —N—, or —O—;
   each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   or two R²¹ on the same N atom are taken together with the N atom to which they are attached to form an unsubstituted or substituted N-containing heterocycle.

In another aspect described herein is a compound of Formula (IIIA2):

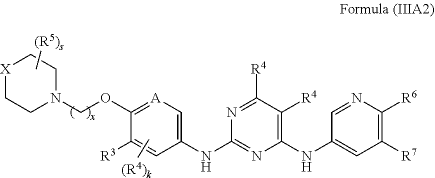

or a pharmaceutically acceptable salt or solvate thereof, wherein:
   x is 1, 2, 3, 4, 5, or 6;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R⁶ and R⁷ are taken together with the intervening carbon atoms connecting R⁶ to R⁷ to form

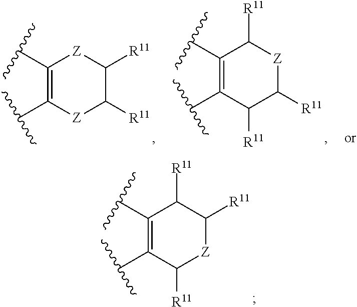

each Z is independently —C(R¹¹)₂, —N(R¹²)—, —O—, —S—, or —SO₂—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In another aspect described herein is a compound of Formula (IIIB):

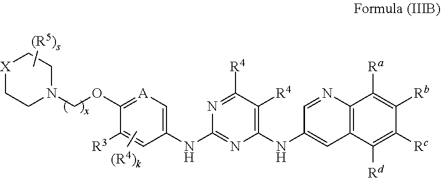

or a pharmaceutically acceptable salt or solvate thereof, wherein:
   x is 1, 2, 3, 4, 5, or 6;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R^{a} is hydrogen, halogen, C₁-C₆alkyl, C₁-C₆fluoroalkyl, C₁-C₆heteroalkyl, C₃-C₈cycloalkyl, C₃-C₈heterocycloalkyl, —CN, —OH, —OR²⁰, or —N(R²¹)₂;
   R^{b} is hydrogen, halogen, C₁-C₆alkyl, C₃-C₈cycloalkyl, C₃-C₈heterocycloalkyl, —CN, —OH, —OR²⁰, or —N(R 21)₂;
   R^{c} is hydrogen, halogen, C₁-C₆alkyl, C₁-C₆fluoroalkyl, C₃-C₈cycloalkyl, C₃-C₈heterocycloalkyl, —CN, —OH, or —N(R²¹)₂;
   R^{d} is hydrogen, halogen, C₁-C₆alkyl, C₁-C₆fluoroalkyl, C₁-C₆heteroalkyl, C₃-C₈cycloalkyl, C₃-C₈heterocycloalkyl, —CN, —OH, —OR²⁰, or —N(R²¹)₂;
   wherein at least one of R^{a}, R^{b}, R^{c}, and R^{d} is not hydrogen;
   each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   or two R²¹ on the same N atom are taken together with the N atom to which they are attached to form an unsubstituted or substituted N-containing heterocycle.

In another aspect described herein is a compound of Formula (IIIC):

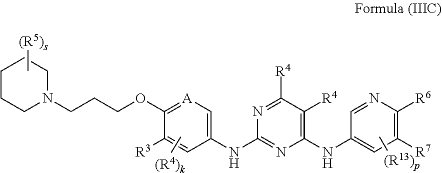

or a pharmaceutically acceptable salt or solvate thereof, wherein:
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   R³ is —O-(unsubstituted or substituted C₁-C₆alkyl);
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
   or R⁶ and R⁷ are taken together with the intervening carbon atoms connecting R⁶ to R⁷ to form

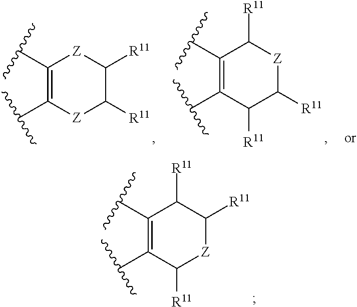

each Z is independently —C(R¹¹)₂, —N(R¹²)—, —O—, or —S—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R², or —C(═O)N(R²¹)₂;
each R¹³ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R¹, —C(═O)N(R²¹)₂, —N(R²¹)₂, —NR²¹C(═O)R¹, —NR²¹CO₂R¹, —SR¹, —S(═O)R²⁰, —SO₂R²¹, or —SO₂N(R²¹)₂;
p is 0, 1, 2, 3, or 4
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In another aspect described herein is a compound of Formula (IV):

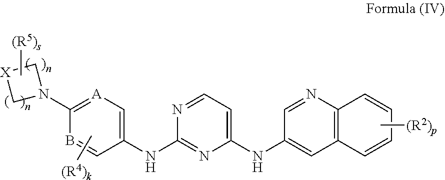

or a pharmaceutically acceptable salt or solvate thereof, wherein:
   each n is independently 1, 2, or 3;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   or both R⁸ are taken together with the carbon to which they are attached to form a unsubstituted or substituted C₃-C₈cycloalkyl, unsubstituted or substituted C₃-C₈heterocycloalkyl;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   B is C(R³) or N;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OH, —OR²⁰, —C(═O)R²¹, —CO₂R²¹, —N(R²¹)₂, —SR²¹, —S(═O)R²⁰, or —SO₂R²;
   p is 0, 1, 2, 3, or 4;
   wherein
   when

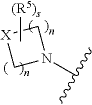

is

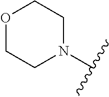 [SMILES: *N1CCOCC1]

and p is 0, at least one of R³ or R⁴ is —O-(substituted C₁-C₆alkyl); or
when

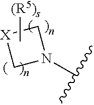

is

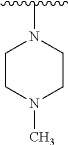 [SMILES: *N1CCN(C)CC1]

and p is 0, at least one of R³ or R⁴ is —O-(unsubstituted or substituted C₁-C₆alkyl),
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In another aspect described herein is a compound of Formula (V):

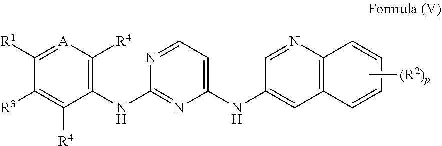

or a pharmaceutically acceptable salt or solvate thereof, wherein:
   R¹ and R³ are taken together with the intervening carbon atoms connecting R¹ to R³ to form a ring A that is

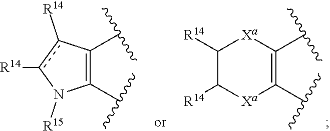

wherein [symbol] indicates a single or double bond;
each R¹⁴ is independently hydrogen, halogen, unsubstituted or substituted C₁-C₀alkyl, unsubstituted or substituted C₁-C₆fluoroalkyl, unsubstituted or substituted C₁-C₆heteroalkyl, substituted or unsubstituted C₁-C₆alkyl-N(R²¹)₂, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R¹⁵ is -substituted or unsubstituted C₁-C₆alkyl, -substituted or unsubstituted C₁-C₆alkyl-N(R²¹)₂, —C(═O)-substituted or unsubstituted C₁-C₆alkyl-N(R²¹)₂, -substituted or unsubstituted C₁-C₆alkyl-carbocycle, or heterocycle;
each X^{a} is independently —C(R¹⁶)₂—, —N(R¹⁷)—, —O—, or —S—;
each R¹⁶ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹⁷ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —C(═O)R²¹, —CO₂R²¹, or —C(═O)N(R²¹)₂;
each R⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
A is C(R⁴) or N;
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²;
p is 0, 1, 2, 3, or 4;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

Also described herein are pharmaceutical compositions comprising a compound described herein, or a pharmaceutically acceptable salt or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered by ophthalmic administration; and/or (g) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the embodiments disclosed herein, the mammal is a human.

In some embodiments, compounds provided herein are orally administered to a human.

Also described herein, in some embodiments, is a compound described herein, or a pharmaceutically acceptable salt or solvate thereof, for use as a medicament.

Also described herein, in some embodiments, is a compound described herein, or a pharmaceutically acceptable salt or solvate thereof, for use in the treatment of a disease or condition in a mammal that would benefit from the modulation of insulin-like growth factor-1 receptor (IGF-1R). In some embodiments, the disease or condition is dependent on IGF-1R activity. In some embodiments, the disease or condition is IGF-1R-mediated. In some embodiments, the disease or condition is selected from thyroid eye disease (TED), also known as thyroid-associated ophthalmopathy (TAO), or Graves' ophthalmopathy or orbitopathy (GO). In some embodiments, the disease or condition is thyroid eye disease (TED).

Also described herein, in some embodiments, is a compound described herein, or a pharmaceutically acceptable salt or solvate thereof, for use in the manufacture of a medicament for the prevention or treatment of a disease or condition ameliorated by the inhibition of IGF-1R.

Also described herein, in some embodiments, is a method of inhibition of IGF-1R comprising contacting IGF-1R with a compound described herein, or a pharmaceutically acceptable salt or solvate thereof

Also described herein, in some embodiments, is a method of treatment of a IGF-1R-mediated disease comprising the administration of a therapeutically effective amount of a compound described herein, or a pharmaceutically acceptable salt or thereof, to a patient in need thereof. In some embodiments, said disease or condition is selected from thyroid eye disease (TED), also known as thyroid-associated ophthalmopathy (TAO), or Graves' ophthalmopathy or orbitopathy (GO). In some embodiments, said disease or condition is thyroid eye disease (TED).

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Compounds disclosed herein are IGF-1R inhibitors activity. In some embodiments, compounds disclosed herein are useful in the treatment of diseases or disorders where inhibition of IGF-1R activity is desired. Also provided is the use of certain compounds disclosed herein for use in the manufacture of a medicament for the treatment of a disease or condition ameliorated by the inhibition of IGF-1R.

### Compounds

Disclosed herein are compounds that inhibit IGF-1R activity. In some embodiments, the IGF-1R inhibitors described herein are useful in the treatment of diseases or disorders in which IGF-1R activity plays an active role. In some embodiments, IGF-1R inhibitors described herein are useful in the treatment of diseases or disorders which would benefit from inhibition of IGF-1R activity.

In one aspect, described herein is a compound of Formula (I):

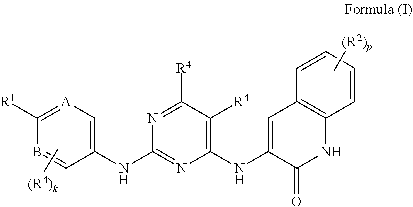

or a pharmaceutically acceptable salt or solvate thereof, wherein:
   R¹ is unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —OR²⁰, —OR²⁰, —C(═O)R²¹, —CO₂R²¹, —C(═O)N(R²¹)₂, —N(R²¹)₂, —NR²¹C(═O)R²¹, —NR²¹CO₂R¹, —SR²¹, —S(═O)R²⁰, —SO₂R²¹, or —SO₂N(R²¹)₂;
   or R¹ is

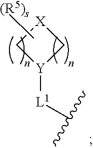

each n is independently 1, 2, or 3;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
Y is CH; and L¹ is —O—, —S—, —N(R²¹)—, -(unsubstituted or substituted C₁-C₆alkyl)-O—, -(unsubstituted or substituted C₁-C₆alkyl)-S—, or -(unsubstituted or substituted C₁-C₆alkyl)-N(R²¹)—;
or Y is N; and L¹ is -(unsubstituted or substituted C₁-C₆alkyl)-O—, -(unsubstituted or substituted C₁-C₆alkyl)-S—, or -(unsubstituted or substituted C₁-C₆alkyl)-N(R²¹)—;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
or both R⁸ are taken together with the carbon to which they are attached to form a unsubstituted or substituted C₃-C₈cycloalkyl, unsubstituted or substituted C₃-C₈heterocycloalkyl;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
B is C(R³) or N;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R¹, —CO₂R²¹, —N(R²¹)₂, —SR²⁰, —S(═O)R²⁰, or —SO₂R²⁰;
or R¹ and R³ are taken together with the intervening carbon atoms connecting R¹ to R³ to form a ring A that is monocyclic C₃-C₈cycloalkyl, monocyclic C₃-C₈heterocycloalkyl, bicyclic C₅-C₁₀cycloalkyl, or bicyclic C₅-C₁₀heterocycloalkyl, wherein ring A is unsubstituted or substituted with 1 or 2 R¹⁴, or 1 or 2 R¹⁵;
or R¹ and R³ are taken together with the intervening carbon atoms connecting R¹ to R³ to form a ring A that is monocyclic C₃-C₈cycloalkyl, bicyclic C₅-C₁₀cycloalkyl, monocyclic C₃-C₈heterocycloalkyl, or bicyclic C₅-C₁₀heterocycloalkyl, wherein ring A is unsubstituted or substituted with one or more groups independently selected from the group consisting of R¹⁴, R¹⁵, R¹⁶, and R¹⁷;
each R¹⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, substituted or unsubstituted C₁-C₆alkyl-N(R²¹)₂, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R¹⁵ is -substituted or unsubstituted C₁-C₆alkyl, -substituted or unsubstituted C₁-C₆alkyl-N(R²¹)₂, —C(═O)-substituted or unsubstituted C₁-C₆alkyl-N(R²¹)₂, -substituted or unsubstituted C₁-C₆alkyl-carbocycle, or heterocycle;
each X^{a} is independently —C(R¹⁶)₂—, —N(R¹⁷)—, —O—, or —S—;
each R¹⁶ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹⁷ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —C(═O)R²¹, —CO₂R²¹, or —C(═O)N(R²¹)₂;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
p is 0, 1, 2, 3, or 4;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

For any and all of the embodiments, substituents are selected from among a subset of the listed alternatives.

In some embodiments, R¹ is unsubstituted or substituted C₃-C₈cycloalkyl, unsubstituted or substituted C₃-C₈heterocycloalkyl, or —OR²⁰; or R¹ is

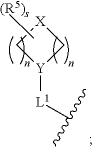

R³ is hydrogen, unsubstituted or substituted C₁-C₆alkyl, unsubstituted or substituted C₁-C₆fluoroalkyl, unsubstituted or substituted C₁-C₆heteroalkyl, or —O-(unsubstituted or substituted C₁-C₆alkyl); or R¹ and R³ are taken together with the intervening carbon atoms connecting R¹ to R³ to form a ring A that is monocyclic C₃-C₈heterocycloalkyl or bicyclic C₅-C₁₀heterocycloalkyl, wherein ring A is substituted with 1 or 2 R¹⁴, or 1 or 2 R¹⁵; or R¹ and R³ are taken together with the intervening carbon atoms connecting R¹ to R³ to form a ring A that is

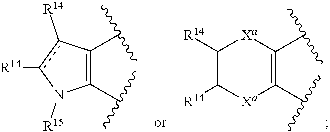

wherein [symbol] indicates a single or double bond; each n is independently 1 or 2; each R⁵ is independently hydrogen, halogen, or unsubstituted or substituted C₁-C₆alkyl; s is 0, 1, or 2; Y is CH; and L¹ is —O— or —N(R²¹)—; X is —C(R⁸)₂—, —N(R⁹)—, or —O—; each R⁸ is independently hydrogen, unsubstituted or substituted C₁-C₆alkyl, unsubstituted or substituted C₁-C₆fluoroalkyl, —C(═O)N(R²¹)₂, —N(R²)₂, or —NR²¹C(═O)R²¹; R⁹ is hydrogen or unsubstituted or substituted C₁-C₆alkyl; each R¹⁴ is independently hydrogen, halogen, unsubstituted or substituted C₁-C₆alkyl, or -(unsubstituted or substituted C₁-C₆alkyl)-N(R²¹)₂; each R¹⁵ is -unsubstituted or substituted C₁-C₆alkyl, -(unsubstituted or substituted C₁-C₆alkyl)-N(R²¹)₂, or —C(═O)-(unsubstituted or substituted C₁-C₆alkyl)-N(R²¹)₂, each X^{a} is independently —C(R¹⁶)₂—, —N(R¹⁷)—, or —O—; each R¹⁶ is independently hydrogen or unsubstituted or substituted C₁-C₆alkyl; each R¹⁷ is independently hydrogen or unsubstituted or substituted C₁-C₆alkyl; and each R²¹ is independently selected from hydrogen or unsubstituted or substituted C₁-C₆alkyl.

In some embodiments, R¹ is

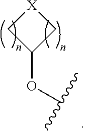

In some embodiments, R¹ is

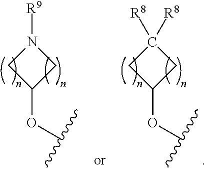

In some embodiments, R¹ is

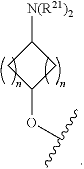

In some embodiments, R¹ is

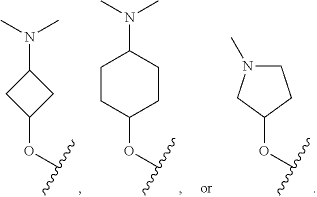

In some embodiments, R¹ is

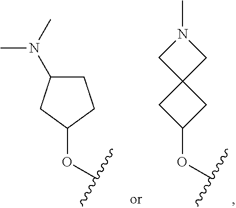

In some embodiments, B is C(R³); and R¹ is substituted C₃-C₈cycloalkyl, unsubstituted or substituted C₃-C₈heterocycloalkyl, or O-(substituted C₁-C₆alkyl).

In some embodiments, R¹ is substituted C₃-C₈heterocycloalkyl, wherein C₃-C₈heterocycloalkyl contains 0-4 N atoms, and 0-2 O atoms. In some embodiments, R¹ is unsubstituted C₃-C₈heterocycloalkyl, wherein C₃-C₈heterocycloalkyl contains 0-4 N atoms, and 0-2 O atoms. In some embodiments, R¹ is unsubstituted or substituted monocyclic C₃-C₈heterocycloalkyl containing at least 1 N atom in the ring. In some embodiments, R¹ is unsubstituted or substituted aziridinyl, unsubstituted or substituted azetidinyl, unsubstituted or substituted morpholinyl, unsubstituted or substituted thiomorpholinyl, unsubstituted or substituted pyrrolidinyl, unsubstituted or substituted piperidinyl, unsubstituted or substituted piperazinyl, or unsubstituted or substituted azepanyl.

In some embodiments, R¹ is substituted with C₁-C₆alkyl, C₁-C₆fluoroalkyl, —N(R²¹)₂, —CN, —OH, or —OR²⁰, wherein C₁-C₆alkyl and C₁-C₆fluoroalkyl can be substituted or unsubstituted. In some embodiments, R¹ is substituted with methyl, ethyl, propyl, iso-propyl, n-butyl, iso-butyl, or tert-butyl, trifluoromethyl, difluoromethyl, fluoromethyl, trifluoroethyl, difluoroethyl, fluoroethyl, —NH₂, —N(CH₃)₂, —CN, —CH₂CN, —OH, or —OCH₃.

In some embodiments, R¹ is

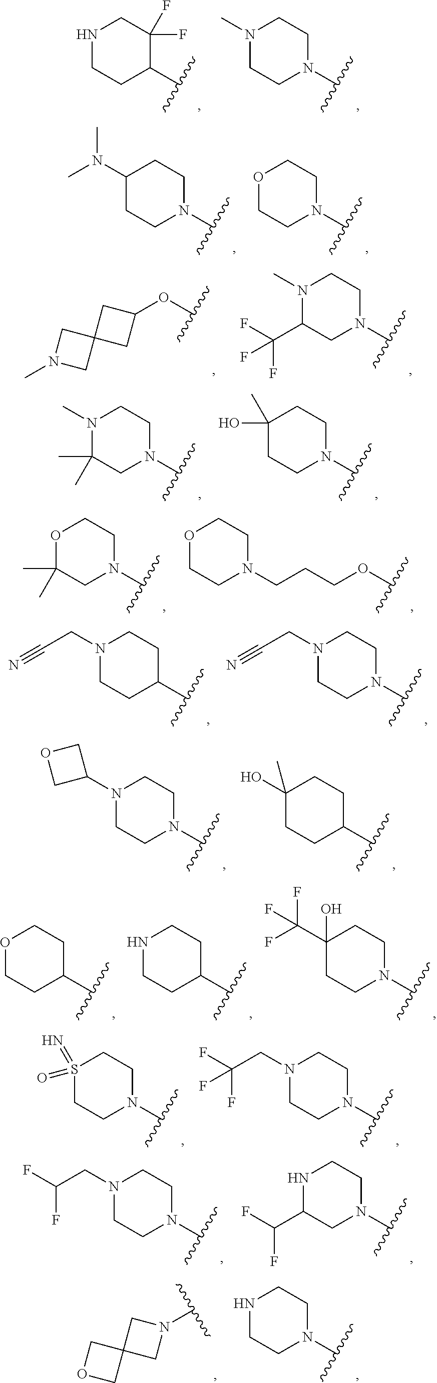

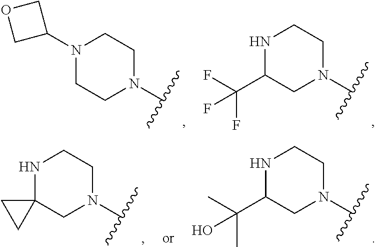

In some embodiments, B is C(R³); and R³ is hydrogen or unsubstituted or substituted C₁-C₆alkoxy. In some embodiments, B is C(R³); and R³ is hydrogen. In some embodiments, B is C(R³); and R³ is —OCH₃.

In some embodiments, B is C(R³); R¹ and R³ are taken together with the intervening carbon atoms connecting R¹ to R³ to form a ring A that is

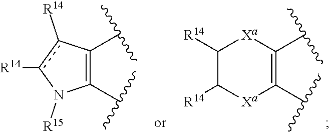

wherein [symbol] indicates a single or double bond; each R⁵ is independently hydrogen, halogen, or unsubstituted or substituted C₁-C₆alkyl; s is 0, 1, or 2; Y is CH; and L¹ is —O— or —N(R²¹)—; X is —C(R⁸)₂—, —N(R⁹)—, or —O—, each R⁸ is independently hydrogen, unsubstituted or substituted C₁-C₆alkyl, unsubstituted or substituted C₁-C₆fluoroalkyl, —C(═O)N(R²¹)₂, —N(R²¹)₂, or —NR²¹C(═O)R²¹; R⁹ is hydrogen or C₁-C₆alkyl; each R¹⁴ is independently hydrogen, halogen, unsubstituted or substituted C₁-C₆alkyl, or -(unsubstituted or substituted C₁-C₆alkyl)-N(R²¹)₂; each R¹⁵ is -(unsubstituted or substituted C₁-C₆alkyl), -(unsubstituted or substituted C₁-C₆alkyl)-N(R²¹)₂, or —C(═O)-(unsubstituted or substituted C₁-C₆alkyl)-N(R²¹)₂; each X^{a} is independently —C(R¹⁶)₂—, —N(R¹⁷)—, or —O—; each R¹⁶ is independently hydrogen or unsubstituted or substituted C₁-C₆alkyl; each R¹⁷ is independently hydrogen or unsubstituted or substituted C₁-C₆alkyl; and each R²¹ is independently selected from hydrogen or unsubstituted or substituted C₁-C₆alkyl.

In some embodiments, B is C(R³); and R¹ and R³ are taken together with the intervening carbon atoms connecting R¹ to R³ to form a ring A that is

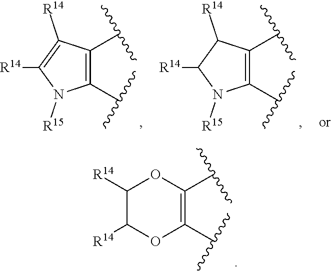

In some embodiments, B is C(R³); and R¹ and R³ are taken together with the intervening carbon atoms connecting R¹ to R³ to form a ring A that is

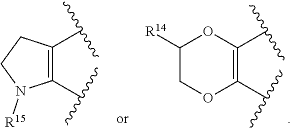

In some embodiments, B is C(R³); and R¹ and R³ are taken together with the intervening carbon atoms connecting R¹ to R³ to form a ring A that is

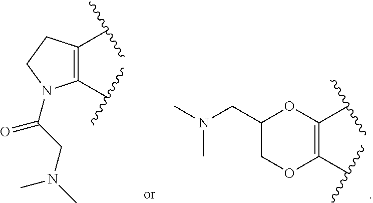 [SMILES: *C1=C(*)N(C(=O)CN(C)C)CC1 ; *C1=C(*)OC(CN(C)C)CO1]

In some embodiments, B is C(R³); R¹ and R³ are taken together with the intervening carbon atoms connecting R¹ to R³ to form a ring A that is monocyclic C₃-C₈heterocycloalkyl or bicyclic C₅-C₁₀heterocycloalkyl, wherein ring A is substituted with 1 or 2 R¹⁴, or 1 or 2 R¹⁵; each R¹⁴ is independently hydrogen, halogen, unsubstituted or substituted C₁-C₆alkyl, or -(unsubstituted or substituted C₁-C₆alkyl)-N(R²¹)₂; and each R¹⁵ is unsubstituted or substituted C₁-C₆alkyl, -(unsubstituted or substituted C₁-C₆alkyl)-N(R²)₂, or —C(═O)-(unsubstituted or substituted C₁-C₆alkyl)-N(R²¹)₂. In some embodiments, B is C(R³); R¹ and R³ are taken together with the intervening carbon atoms connecting R¹ to R³ to form a ring A that is monocyclic C₃-C₈heterocycloalkyl substituted with —C(═O)-(unsubstituted or substituted C₁-C₆alkyl)-N(R²)₂ or -(unsubstituted or substituted C₁-C₆alkyl)-N(R²¹)₂. In some embodiments, B is C(R³); R¹ and R³ are taken together with the intervening carbon atoms connecting R¹ to R³ to form a ring A that is bicyclic C₅-C₁₀heterocycloalkyl substituted with C₁-C₆alkyl.

In some embodiments, B is C(R³); R¹ and R³ are taken together with the intervening carbon atoms connecting R¹ to R³ to form a ring A that is a bicyclic C₅-C₁₀heterocycloalkyl that is a fused bicyclic C₅-C₁₀heterocycloalkyl, bridged bicyclic C₅-C₁₀heterocycloalkyl, or spiro bicyclic C₅-C₁₀heterocycloalkyl.

In some embodiments, B is C(R³); R¹ and R³ are taken together with the intervening carbon atoms connecting R¹ to R³ to form a ring A that is a bicyclic C₅-C₁₀heterocycloalkyl that is a spiro bicyclic C₅-C₁₀heterocycloalkyl containing at least one N atom in the ring.

In some embodiments, B is C(R³); and R¹ and R³ are taken together with the intervening carbon atoms connecting R¹ to R³ to form a ring A, wherein

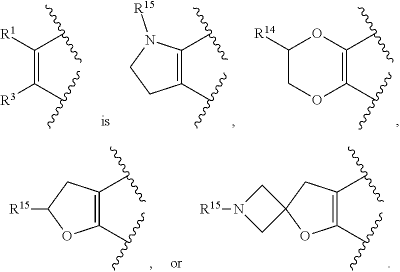

In some embodiments, B is C(R³); and R¹ and R³ are taken together with the intervening carbon atoms connecting R¹ to R³ to form a ring A, wherein

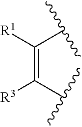

is

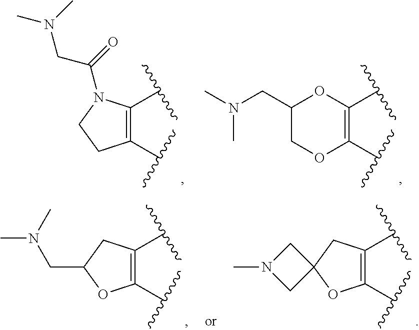

In some embodiments, B is C(R³); and R¹ is substituted C₃-C₈cycloalkyl, unsubstituted or substituted C₃-C₈heterocycloalkyl, or O-(substituted C₁-C₆alkyl).

In some embodiments, each R² is independently hydrogen, halogen, unsubstituted or substituted C₁-C₆alkyl, unsubstituted or substituted C₁-C₆fluoroalkyl, or unsubstituted or substituted C₁-C₆heteroalkyl; p is 0, 1, or 2; and each R⁴ is independently hydrogen, unsubstituted or substituted C₁-C₆alkyl, or unsubstituted or substituted C₁-C₆alkoxy. In some embodiments, each R² is independently hydrogen, halogen, —CH₃, —CF₃, —CHF₂, or —CH₂F; p is 0, 1, or 2; and each R⁴ is independently hydrogen, —CH₃, or —OCH₃. In some embodiments, each R² is hydrogen or halogen; p is 0, 1, or 2; and each R⁴ is independently hydrogen or —OCH₃. In some embodiments, R² is —F; p is 1; and each R⁴ is hydrogen.

In some embodiments, each R² is independently hydrogen, —F, —Cl, or —Br. In some embodiments, R² is —F.

In some embodiments,

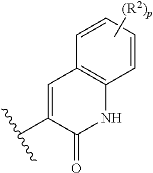

is

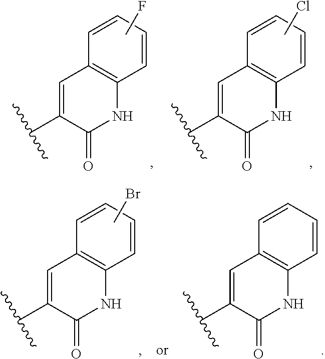

In some embodiments,

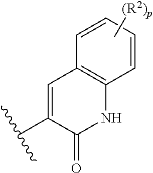

is

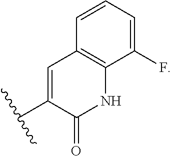 [SMILES: *c1cc2cccc(F)c2[nH]c1=O]

In one aspect, described herein is a compound of Formula (II):

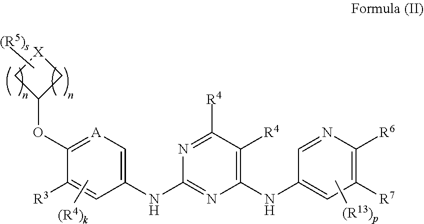

or a pharmaceutically acceptable salt or solvate thereof, wherein:
   each n is independently 1, 2, or 3;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   or both R⁸ are taken together with the carbon to which they are attached to form a unsubstituted or substituted C₃-C₈cycloalkyl, unsubstituted or substituted C₃-C₈heterocycloalkyl;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
   or R⁶ and R⁷ are taken together with the intervening carbon atoms connecting R⁶ to R⁷ to form

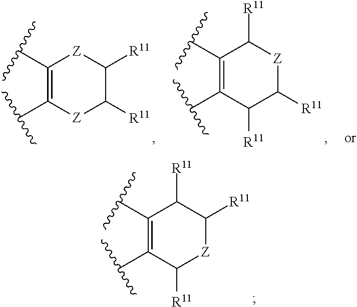

each Z is independently —C(R¹¹)₂, —N(R¹²)—, —O—, —S—, or —SO₂—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R², or —C(═O)N(R²¹)₂;
each R¹³ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R¹, —C(═O)N(R²¹)₂, —N(R²¹)₂, —NR²¹C(═O)R¹, —NR²¹CO₂R¹, —SR¹, —S(═O)R²⁰, —SO₂R²¹, or —SO₂N(R²¹)₂;
p is 0, 1, 2, 3, or 4
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted heterocycle containing 1 or 2 N atoms, 0 or 1 O atoms, and 0 or 1 S, S(O), or S(O)₂.

In some embodiments, n is 1 or 2; X is —C(R⁸)₂— or —N(R⁹)—; each R⁸ is independently hydrogen, C₁-C₆alkyl, or —N(R²¹)₂; R⁹ is hydrogen or unsubstituted or substituted C₁-C₆alkyl; and s is 0.

In some embodiments, n is 1 or 2; X is —C(R⁸)₂—; each R⁸ is independently hydrogen or —N(R²¹)₂; s is 0; and R²¹ is hydrogen or unsubstituted or substituted C₁-C₆ alkyl, or two R²¹ on the same N atom are taken together with the N atom to which they are attached to form an unsubstituted or substituted heterocycle containing 1 or 2 N atoms, 0 or 1 O atoms, and 0 or 1 S, S(O), or S(O)₂.

In some embodiments, X is —CHN(R²¹)₂—; each R²¹ is independently unsubstituted or substituted C₁-C₆ alkyl, or two R²¹ on the same N atom are taken together with the N atom to which they are attached to form an unsubstituted or substituted monocyclic or bicyclic C₂-C₈heterocycloalkyl containing 1 or 2 N atoms, 0 or 1 O atoms, and 0 or 1 S, S(O), or S(O)₂ in the ring.

In some embodiments, X is —CHN(R²¹)₂—; each R²¹ is independently unsubstituted or substituted C₁-C₄ alkyl, or two R²¹ on the same N atom are taken together with the N atom to which they are attached to form an unsubstituted or substituted monocyclic or bridged bicyclic C₂-C₈heterocycloalkyl containing 1 or 2 N atoms, 0 or 1 O atoms, and 0 or 1 S, S(O), or S(O)₂ in the ring.

In some embodiments, R¹ is

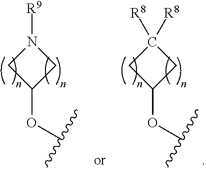

In some embodiments, R¹ is

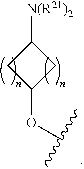

In some embodiments, R¹ is

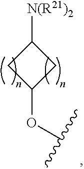

wherein two R²¹ on the same N atom are taken together with the N atom to which they are attached to form an unsubstituted or substituted monocyclic heterocycle containing 1 or 2 N atoms, 0 or 1 O atoms, O or 1 S atoms, or S(O)₂. In some embodiments, R¹ is

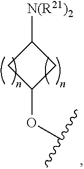

wherein two R²¹ on the same N atom are taken together with the N atom to which they are attached to form an unsubstituted or substituted monocyclic C₂-C₈heterocycloalkyl containing 1 or 2 N atoms, 0 or 1 O atoms, O or 1 S atoms, or S(O)₂. In some embodiments, R¹ is

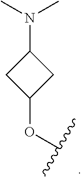

In some embodiments, R¹ is

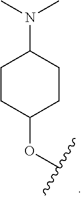 [SMILES: *OC1CCC(N(C)C)CC1]

In some embodiments,

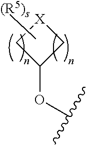

is

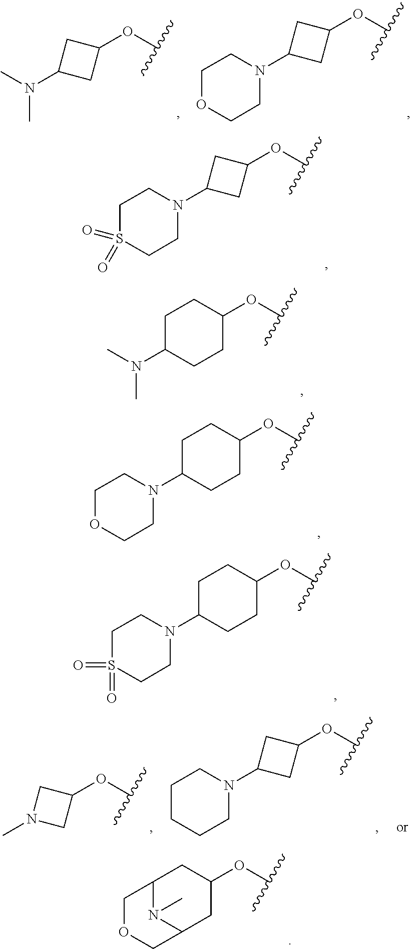

In some embodiments, each R⁴ is independently hydrogen, halogen, unsubstituted or substituted C₁-C₆alkyl, unsubstituted or substituted C₁-C₆fluoroalkyl, or unsubstituted or substituted C₁-C₆alkoxy. In some embodiments, each R⁴ is independently hydrogen, halogen, unsubstituted C₁-C₆alkyl, unsubstituted C₁-C₆fluoroalkyl, or unsubstituted C₁-C₆alkoxy. In some embodiments, each R⁴ is independently hydrogen, —F, —Cl, —Br, —I, methyl, ethyl, propyl, iso-propyl, n-butyl, iso-butyl, tert-butyl, trifluoromethyl, difluoromethyl, fluoromethyl, trifluoroethyl, difluoroethyl, fluoroethyl, methoxy, ethoxy, n-propoxy, isopropoxy, n-butoxy, iso-butoxy, sec-butoxy, or tert-butoxy. In some embodiments, each R⁴ is independently hydrogen or C₁-C₆alkoxy. In some embodiments, each R⁴ is independently hydrogen, methoxy, or ethoxy. In some embodiments, R⁴ is hydrogen. In some embodiments, R⁴ is methoxy.

In some embodiments, each R⁴ is independently hydrogen, halogen, unsubstituted or substituted C₁-C₆alkyl, or unsubstituted or substituted C₁-C₆alkoxy; and R³ is —O(unsubstituted or substituted C₁-C₆ alkyl). In some embodiments, R⁴ is hydrogen, and R³ is —OCH₃. In some embodiments, R⁴ is hydrogen, and R³ is hydrogen.

In some embodiments, R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²; or R⁶ and R⁷ are taken together with the intervening carbon atoms connecting R⁶ to R⁷ to form

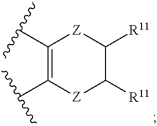

each Z is independently —C(R¹¹)₂, —N(R¹²)—, or —O—; each R¹¹ is independently hydrogen, halogen, unsubstituted or substituted C₁-C₆alkyl, unsubstituted or substituted C₁-C₆fluoroalkyl, —CN, —OH, —OR²⁰, —N(R²¹)₂, or —NR²¹C(═O)R²¹; each R¹³ is independently hydrogen, halogen, unsubstituted or substituted C₁-C₆alkyl, —CN, —OH, —OR²⁰, —CO₂R²¹, —C(═O)N(R²¹)₂, —N(R²¹)₂, or —NR²¹C(═O)R²¹; and p is 0, 1, or 2.

In some embodiments, R⁶ and R⁷ are taken together with the intervening carbon atoms connecting R⁶ to R⁷ to form a ring B that is bicyclic C₅-C₁₀heterocycloalkyl that is a fused bicyclic C₅-C₁₀heterocycloalkyl, bridged bicyclic C₅-C₁₀heterocycloalkyl, or spiro bicyclic C₅-C₁₀heterocycloalkyl; or R⁶ and R⁷ are taken together with the intervening carbon atoms connecting R⁶ to R⁷ to form a ring B that is bicyclic C₅-C₁₀cycloalkyl that is a fused bicyclic C₅-C₁₀cycloalkyl, bridged bicyclic C₅-C₁₀cycloalkyl, or spiro bicyclic C₅-C₁₀cycloalkyl.

In some embodiments, R⁶ and R⁷ are taken together with the intervening carbon atoms connecting R⁶ to R⁷ to form a ring B that is bicyclic C₅-C₁₀heterocycloalkyl that is a fused bicyclic C₅-C₁₀heterocycloalkyl or a bicyclic C₅-C₁₀cycloalkyl that is a fused bicyclic C₅-C₁₀cycloalkyl. In some embodiments, R⁶ and R⁷ are taken together with the intervening carbon atoms connecting R⁶ to R⁷ to form a ring B that is a fused bicyclic C₅-C₁₀heterocycloalkyl containing 0-2 N atoms, 0-2 O atoms, or 0-1 S atoms in the ring.

In some embodiments, R⁶ and R⁷ are taken together with the intervening carbon atoms connecting R⁶ to R⁷ to form a ring B that is bicyclic C₅-C₁₀heterocycloalkyl that is a spiro bicyclic C₅-C₁₀heterocycloalkyl or a bicyclic C₅-C₁₀cycloalkyl that is a spiro bicyclic C₅-C₁₀cycloalkyl. In some embodiments, R⁶ and R⁷ are taken together with the intervening carbon atoms connecting R⁶ to R⁷ to form a ring B that is a spiro bicyclic C₅-C₁₀heterocycloalkyl containing 0-2 N atoms, 0-2 O atoms, or 0-1 S atoms in the ring. In some embodiments, R⁶ and R⁷ are taken together with the intervening carbon atoms connecting R⁶ to R⁷ to form a ring B that is a spiro bicyclic C₅-C₁₀cycloalkyl.

In some embodiments, R⁶ and R⁷ are taken together with the intervening carbon atoms connecting R⁶ to R⁷ to form a ring B that is monocyclic C₃-C₈cycloalkyl, monocyclic C₃-C₈ aryl, or monocyclic C₃-C₈ heteroaryl, wherein ring B is unsubstituted or substituted with 1 or 2 halogen, unsubstituted or substituted C₁-C₆alkyl, unsubstituted or substituted C₁-C₆fluoroalkyl, —CN, —OH, —OR²⁰, —N(R²¹)₂, or —NR²¹C(═O)R²¹ or R⁶ and R⁷ are taken together with the intervening carbon atoms connecting R⁶ to R⁷ to form

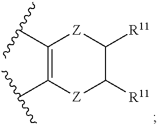

each Z is —O—; each R¹³ is independently hydrogen, halogen, unsubstituted or substituted C₁-C₆alkyl, —CN, —OH, —OR²⁰, —CO₂R²¹, —C(═O)N(R²¹)₂; and p is 0, 1, or 2.

In some embodiments, R⁶ and R⁷ are taken together with the intervening carbon atoms connecting R⁶ to R⁷ to form a ring B that is monocyclic C₃-C₈cycloalkyl or monocyclic C₃-C₈ aryl, wherein ring B is unsubstituted or substituted with 1 or 2 halogen, C₁-C₆alkyl, C₁-C₆fluoroalkyl, —CN, —OH, —OR²⁰, —N(R²¹)₂, or —NR²¹C(═O)R²¹. In some embodiments, R⁶ and R⁷ are taken together with the intervening carbon atoms connecting R⁶ to R⁷ to form a ring B that is cyclopropyl, cyclobutyl, cyclopentyl, cyclohexyl, cycloheptyl, cyclooctyl, wherein ring B is unsubstituted or substituted with 1 or 2 halogen, C₁-C₆alkyl, C₁-C₆fluoroalkyl, —CN, —OH, —O(C₁-C₆alkyl), or —N(C₁-C₆alkyl)₂. In some embodiments, R⁶ and R⁷ are taken together with the intervening carbon atoms connecting R⁶ to R⁷ to form a ring B that is phenyl, wherein Ring B is unsubstituted or substituted with 1 or 2 halogen, C₁-C₆alkyl, C₁-C₆fluoroalkyl, —CN, —OH, —O(C₁-C₆alkyl), or —N(C₁-C₆alkyl)₂.

In some embodiments, R⁶ and R⁷ are taken together with the intervening carbon atoms connecting R⁶ to R⁷ to form a ring B that is monocyclic C₃-C₈heteroaryl, wherein ring B is unsubstituted or substituted with 1 or 2 halogen, C₁-C₆alkyl, C₁-C₆fluoroalkyl, —CN, —OH, —OR²⁰, —N(R²¹)₂, or —NR²¹C(═O)R²¹. In some embodiments, R⁶ and R⁷ are taken together with the intervening carbon atoms connecting R⁶ to R⁷ to form a ring B that is furanyl, thienyl, pyrrolyl, oxazolyl, thiazolyl, imidazolyl, pyrazolyl, triazolylene, tetrazolyl, isoxazolyl, isothiazolyl, oxadiazolyl, thiadiazolyl, pyridinyl, pyrimidinyl, pyrazinyl, pyridazinyl, and triazinyl, wherein ring B is unsubstituted or substituted with 1 or 2 halogen, C₁-C₆alkyl, C₁-C₆fluoroalkyl, —CN, —OH, —O(C₁-C₆alkyl), or —N(C₁-C₆alkyl)₂. In some embodiments, R⁶ and R⁷ are taken together with the intervening carbon atoms connecting R⁶ to R⁷ to form a ring B that is pyrrolyl, pyridinyl, pyrimidinyl, pyrazinyl, or pyridazinyl, wherein Ring B is unsubstituted or substituted with 1 or 2 halogen, C₁-C₆alkyl, C₁-C₆fluoroalkyl, —CN, —OH, —O(C₁-C₆alkyl), or —N(C₁-C₆alkyl)₂.

In some embodiments, R⁶ and R⁷ are taken together with the intervening carbon atoms connecting R⁶ to R⁷ to form

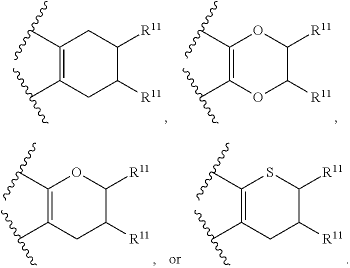

In some embodiments,

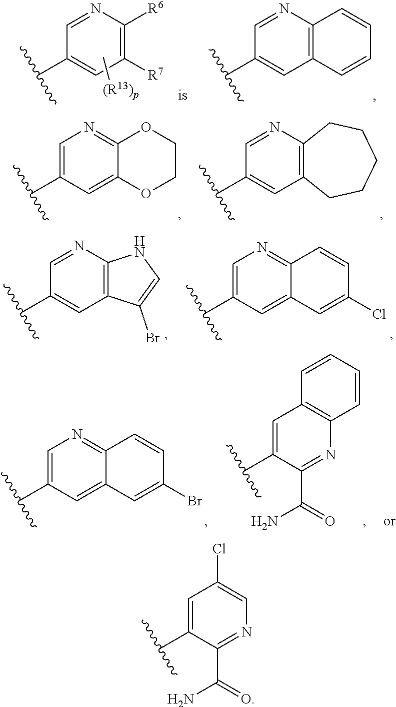

In another aspect described herein is a compound of Formula (IIIA1):

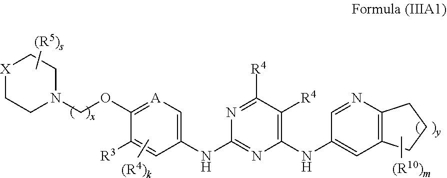

or a pharmaceutically acceptable salt or solvate thereof, wherein:
   x is 1, 2, 3, 4, 5, or 6;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R¹, —C(═O)N(R²¹)₂, —N(R²¹)₂, —NR²¹C(═O)R²¹, —NR²¹CO₂R², —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   y is 1, 2, or 3;
   each R¹⁰ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —CO₂R²¹, —C(═O)N(R²¹)₂, —N(R²¹)₂, —NR²¹C(═O)R²¹, —SR²⁰, —S(═O)R²⁰, —SO₂R²⁰ or —SO₂N(R²¹);
   m is 0, 1, 2, 3, or 4;
   wherein two R¹⁰ can be optionally connected by L;
   L is C₁-C₆ alkyl, —N—, or —O—;
   each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   or two R²¹ on the same N atom are taken together with the N atom to which they are attached to form an unsubstituted or substituted N-containing heterocycle.

In some embodiments, x is 1, 2, 3, or 4; R³ is hydrogen, halogen, unsubstituted or substituted C₁-C₆alkyl, or —O(unsubstituted or substituted C₁-C₆alkyl); X is —C(R⁸)₂—, —O—, or —SO₂—; and each R⁸ is independently hydrogen, halogen, unsubstituted or substituted C₁-C₆alkyl.

In some embodiments, R³ is hydrogen, halogen, unsubstituted or substituted C₁-C₆alkyl, or unsubstituted or substituted C₁-C₆alkoxy. In some embodiments, R³ is hydrogen, unsubstituted or substituted C₁-C₆alkyl, or unsubstituted or substituted C₁-C₆alkoxy. In some embodiments, R³ is hydrogen, methyl, ethyl, propyl, iso-propyl, n-butyl, iso-butyl, tert-butyl, methoxy, ethoxy, n-propoxy, isopropoxy, n-butoxy, iso-butoxy, sec-butoxy, or tert-butoxy. In some embodiments, R³ is hydrogen, methyl, or methoxy. In some embodiments, R³ is hydrogen. In some embodiments, R³ is methoxy.

In some embodiments, each R⁴ is independently hydrogen, halogen, unsubstituted or substituted C₁-C₆alkyl, or unsubstituted or substituted C₁-C₆alkoxy. In some embodiments, each R⁴ is independently unsubstituted or substituted C₁-C₆alkyl, or unsubstituted or substituted C₁-C₆alkoxy. In some embodiments, each R⁴ is independently hydrogen, methyl, ethyl, propyl, iso-propyl, n-butyl, iso-butyl, tert-butyl, methoxy, ethoxy, n-propoxy, isopropoxy, n-butoxy, iso-butoxy, sec-butoxy, or tert-butoxy. In some embodiments, each R⁴ is hydrogen or methoxy. In some embodiments, R⁴ is hydrogen. In some embodiments, R⁴ is methoxy.

In some embodiments,

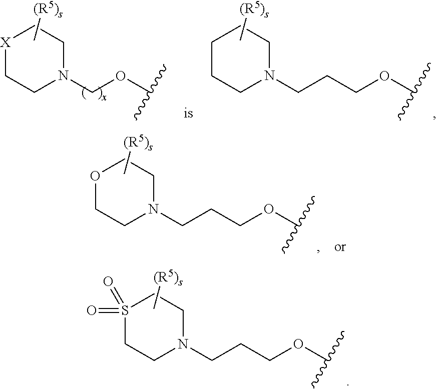

In some embodiments, y is 1 or 2; each R¹⁰ is independently hydrogen, halogen, C₁-C₆alkyl, C₁-C₆fluoroalkyl, —OH, —OR²⁰, or —N(R²¹)₂; m is 0, 1, 2, 3, or 4; wherein two R¹⁰ can be optionally connected by L; and L is C₁-C₆ alkyl or —O—.

In some embodiments, y is 1 or 2; each R¹⁰ is independently hydrogen, halogen, or unsubstituted or substituted C₁-C₆alkyl; m is 0, 1, 2, 3, or 4; wherein two R¹⁰ can be optionally connected by L; and L is unsubstituted or substituted C₁-C₆ alkyl or —O—.

In some embodiments, each R¹⁰ is independently hydrogen, halogen, or unsubstituted or substituted C₁-C₆alkyl; and two R¹⁰ are connected by L, wherein L is unsubstituted or substituted C₁-C₆ alkyl, —N—, or —O—.

In some embodiments,

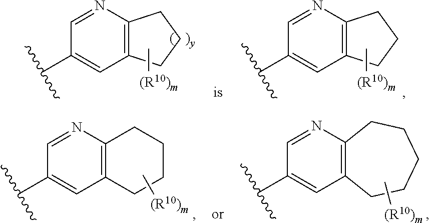

In some embodiments,

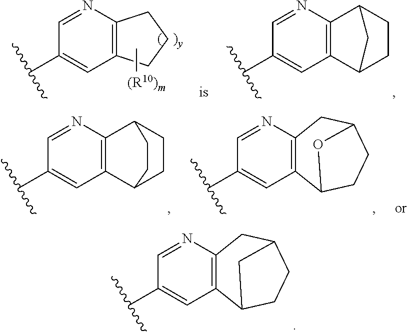

In some embodiments,

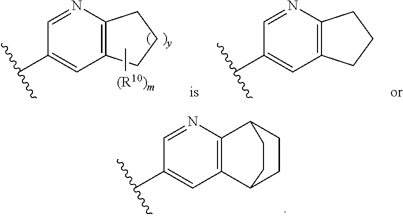

In another aspect described herein is a compound of Formula (IIIA2):

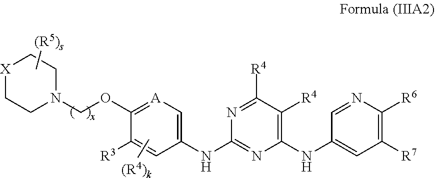

or a pharmaceutically acceptable salt or solvate thereof, wherein:
   x is 1, 2, 3, 4, 5, or 6;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²¹;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R⁶ and R⁷ are taken together with the intervening carbon atoms connecting R⁶ to R⁷ to form

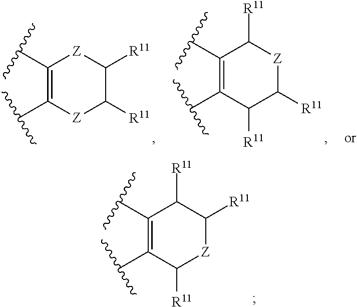

each Z is independently —C(R¹¹)₂, —N(R¹²)—, —O—, —S—, or —SO₂—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R², —CO₂R²¹, or —C(═O)N(R²¹)₂;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In some embodiments, x is 1, 2, 3, or 4; R³ is hydrogen, halogen, unsubstituted or substituted C₁-C₆alkyl, or —O(unsubstituted or substituted C₁-C₆alkyl); X is —C(R⁸)₂—, —O—, or —SO₂—; and each R⁸ is independently hydrogen, halogen, unsubstituted or substituted C₁-C₆alkyl.

In some embodiments, R³ is hydrogen, halogen, unsubstituted or substituted C₁-C₆alkyl, or unsubstituted or substituted C₁-C₆alkoxy. In some embodiments, R³ is hydrogen, unsubstituted or substituted C₁-C₆alkyl, or unsubstituted or substituted C₁-C₆alkoxy. In some embodiments, R³ is hydrogen, methyl, ethyl, propyl, iso-propyl, n-butyl, iso-butyl, tert-butyl, methoxy, ethoxy, n-propoxy, isopropoxy, n-butoxy, iso-butoxy, sec-butoxy, or tert-butoxy. In some embodiments, R³ is hydrogen, methyl, or methoxy. In some embodiments, R³ is hydrogen. In some embodiments, R³ is methoxy.

In some embodiments, each R⁴ is independently hydrogen, halogen, unsubstituted or substituted C₁-C₆alkyl, or unsubstituted or substituted C₁-C₆alkoxy. In some embodiments, each R⁴ is independently unsubstituted or substituted C₁-C₆alkyl, or unsubstituted or substituted C₁-C₆alkoxy. In some embodiments, each R⁴ is independently hydrogen, methyl, ethyl, propyl, iso-propyl, n-butyl, iso-butyl, tert-butyl, methoxy, ethoxy, n-propoxy, isopropoxy, n-butoxy, iso-butoxy, sec-butoxy, or tert-butoxy. In some embodiments, each R⁴ is hydrogen or methoxy. In some embodiments, R⁴ is hydrogen. In some embodiments, R⁴ is methoxy.

In some embodiments,

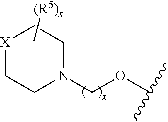

is

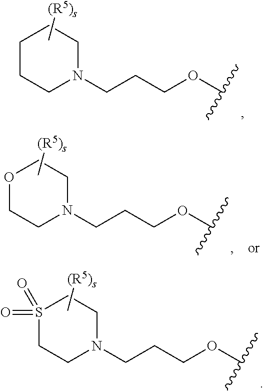

In some embodiments, R⁶ and R⁷ are taken together with the intervening carbon atoms connecting R⁶ to R⁷ to form

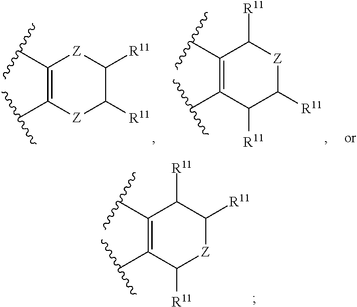

each Z is independently —C(R¹¹)₂, —N(R¹²)—, —O—, —S—, or —SO₂—; each R¹¹ is independently hydrogen, halogen, unsubstituted or substituted C₁-C₆alkyl, unsubstituted or substituted C₁-C₆fluoroalkyl, oxo, —CN, —OH, —OR²⁰, or —N(R²¹)₂; and each R¹² is hydrogen or unsubstituted or substituted C₁-C₆alkyl.

In some embodiments, R⁶ and R⁷ are taken together with the intervening carbon atoms connecting R⁶ to R⁷ to form

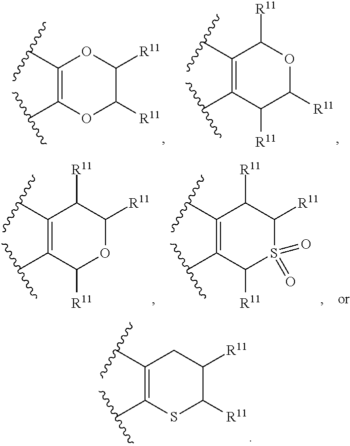

In some embodiments, R⁶ and R⁷ are taken together with the intervening carbon atoms connecting R⁶ to R⁷ to form

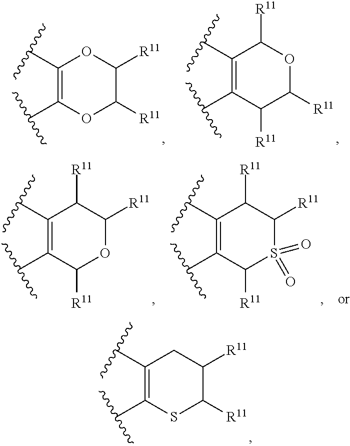

wherein each R¹¹ is independently hydrogen, oxo, or unsubstituted or substituted C₁-C₆alkyl.

In some embodiments, each R¹¹ is independently hydrogen or methyl.

In another aspect described herein is a compound of Formula (IIIB):

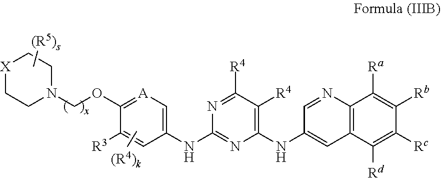

or a pharmaceutically acceptable salt or solvate thereof, wherein:
   x is 1, 2, 3, 4, 5, or 6;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R^{a} is hydrogen, halogen, C₁-C₆alkyl, C₁-C₆fluoroalkyl, C₁-C₆heteroalkyl, C₃-C₈cycloalkyl, C₃-C₈heterocycloalkyl, —CN, —OH, —OR²⁰, or —N(R²¹)₂;
   R^{b} is hydrogen, halogen, C₁-C₆alkyl, C₃-C₈cycloalkyl, C₃-C₈heterocycloalkyl, —CN, —OH, —OR²⁰, or —N(R²¹)₂;
   R^{c} is hydrogen, halogen, C₁-C₆alkyl, C₁-C₆fluoroalkyl, C₃-C₈cycloalkyl, C₃-C₈heterocycloalkyl, —CN, —OH, or —N(R²¹)₂;
   R^{d} is hydrogen, halogen, C₁-C₆alkyl, C₁-C₆fluoroalkyl, C₁-C₆heteroalkyl, C₃-C₈cycloalkyl, C₃-C₈heterocycloalkyl, —CN, —OH, —OR²⁰, or —N(R²¹)₂;
   wherein at least one of R^{a}, R^{b}, R^{c}, and R^{d} is not hydrogen;
   each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
   or two R²¹ on the same N atom are taken together with the N atom to which they are attached to form an unsubstituted or substituted N-containing heterocycle.

In some embodiments, x is 1, 2, 3, or 4; R³ is hydrogen, halogen, unsubstituted or substituted C₁-C₆alkyl, or —O(unsubstituted or substituted C₁-C₆alkyl); X is —C(R⁸)₂—, —O—, or —SO₂—; and each R⁸ is independently hydrogen, halogen, unsubstituted or substituted C₁-C₆alkyl.

In some embodiments,

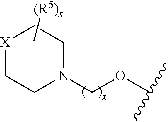

is

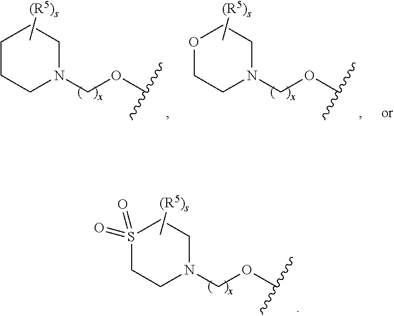

In some embodiments,

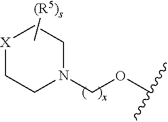

is

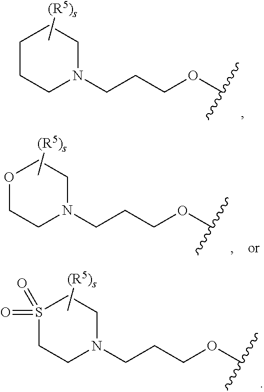

In some embodiments, R^{a} is hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, or —N(R²¹)₂. In some embodiments, R^{a} is hydrogen, halogen, unsubstituted or substituted C₁-C₆alkyl, unsubstituted or substituted C₁-C₆fluoroalkyl, —OH, or —O(unsubstituted or substituted C₁-C₆alkyl). In some embodiments, R^{a} is hydrogen. In some embodiments, R^{a} is —F, —Cl, —Br, or —I. In some embodiments, R^{a} is —OH. In some embodiments, R^{a} is methyl, ethyl, propyl, iso-propyl, n-butyl, iso-butyl, tert-butyl, trifluoromethyl, difluoromethyl, fluoromethyl, trifluoroethyl, difluoroethyl, fluoroethyl, methoxy, ethoxy, n-propoxy, isopropoxy, n-butoxy, iso-butoxy, sec-butoxy, or tert-butoxy.

In some embodiments, R^{b} is hydrogen, halogen, unsubstituted or substituted C₁-C₆alkyl, unsubstituted or substituted C₃-C₈cycloalkyl, unsubstituted or substituted C₃-C₈heterocycloalkyl, —CN, —OH, —OR²⁰, or —N(R²¹)₂. In some embodiments, R^{b} is hydrogen, halogen, unsubstituted or substituted C₁-C₆alkyl, unsubstituted or substituted C₃-C₈cycloalkyl, —OH, or O—(C₁-C₆alkyl). In some embodiments, R^{b} is hydrogen. In some embodiments, R^{b} is —F, —Cl, —Br, or —I. In some embodiments, R^{b} is —OH. In some embodiments, R^{b} is methyl, ethyl, propyl, iso-propyl, n-butyl, iso-butyl, tert-butyl, methoxy, ethoxy, n-propoxy, isopropoxy, n-butoxy, iso-butoxy, sec-butoxy, or tert-butoxy. In some embodiments, R^{b} is cyclopropyl, cyclobutyl, cyclopentyl, or cyclohexyl.

In some embodiments, R^{c} is hydrogen, halogen, unsubstituted or substituted C₁-C₆alkyl, unsubstituted or substituted C₁-C₆fluoroalkyl, unsubstituted or substituted C₃-C₈cycloalkyl, unsubstituted or substituted C₃-C₈heterocycloalkyl, —CN, —OH, or —N(R²¹)₂. In some embodiments, R^{c} is hydrogen, halogen, unsubstituted or substituted C₁-C₆alkyl, unsubstituted or substituted C₁-C₆fluoroalkyl, unsubstituted or substituted C₃-C₈cycloalkyl, —CN, or —OH. In some embodiments, R^{c} is hydrogen. In some embodiments, R^{c} is —F, —Cl, —Br, or —I. In some embodiments, R^{c} is —OH. In some embodiments, R^{c} is —CN. In some embodiments, R^{c} is methyl, ethyl, propyl, iso-propyl, n-butyl, iso-butyl, tert-butyl, trifluoromethyl, difluoromethyl, fluoromethyl, trifluoroethyl, difluoroethyl, or fluoroethyl. In some embodiments, R^{c} is cyclopropyl, cyclobutyl, cyclopentyl, or cyclohexyl.

In some embodiments, R^{d} is hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, or —N(R²¹)₂. In some embodiments, R^{d} is hydrogen, halogen, unsubstituted or substituted C₁-C₆alkyl, or unsubstituted or substituted C₁-C₆fluoroalkyl. In some embodiments, R^{d} is hydrogen. In some embodiments, R^{d} is —F, —Cl, —Br, or —I. In some embodiments, R^{d} is methyl, ethyl, propyl, iso-propyl, n-butyl, iso-butyl, tert-butyl, trifluoromethyl, difluoromethyl, fluoromethyl, trifluoroethyl, difluoroethyl, or fluoroethyl.

In some embodiments, R^{a} is hydrogen, halogen, unsubstituted or substituted C₁-C₆alkyl, unsubstituted or substituted C₁-C₆fluoroalkyl, or —OR²⁰; R^{b} is hydrogen, halogen, unsubstituted or substituted C₁-C₆alkyl, unsubstituted or substituted C₃-C₈cycloalkyl, —OR²⁰, or —N(R²¹)₂; R^{c} is hydrogen, halogen, unsubstituted or substituted C₁-C₆alkyl, unsubstituted or substituted C₁-C₆fluoroalkyl, or —CN; and R^{d} is hydrogen, halogen, or unsubstituted or substituted C₁-C₆alkyl.

In some embodiments, R^{a} is hydrogen, halogen, unsubstituted or substituted C₁-C₆alkyl, —CF₃, —OCH₃, or —OCF₃; R^{b} is hydrogen, halogen, unsubstituted or substituted C₁-C₆alkyl, unsubstituted or substituted C₃-C₆cycloalkyl, —OCH₃, or —OCF₃; R^{c} is hydrogen, halogen, unsubstituted or substituted C₁-C₆alkyl, —CF₃, or —CN; and R^{d} is hydrogen, halogen, or unsubstituted or substituted C₁-C₆alkyl.

In some embodiments, at least one of R^{a}, R^{b}, R^{c}, or R^{d} is halogen.

In another aspect described herein is a compound of Formula (IIIC):

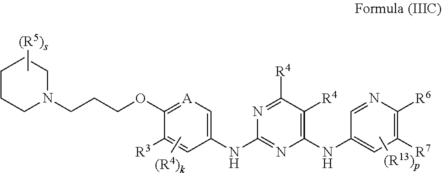

or a pharmaceutically acceptable salt or solvate thereof, wherein:
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   R³ is —O-(unsubstituted or substituted C₁-C₆alkyl);
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
   or R⁶ and R⁷ are taken together with the intervening carbon atoms connecting R⁶ to R⁷ to form

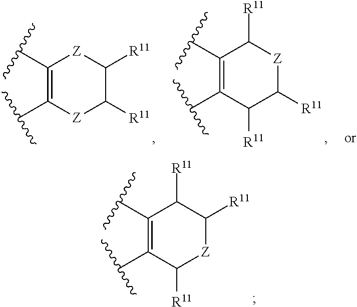

each Z is independently —C(R¹¹)₂, —N(R¹²)—, —O—, or —S—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R², or —C(═O)N(R²¹)₂;
each R¹³ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R¹, —C(═O)N(R²¹)₂, —N(R²¹)₂, —NR²¹C(═O)R¹, —NR²¹CO₂R¹, —SR¹, —S(═O)R²⁰, —SO₂R²¹, or —SO₂N(R²¹)₂;
p is 0, 1, 2, 3, or 4
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In some embodiments, s is 0 and k is 0. In some embodiments, s is 0. In some embodiments, k is 0.

In some embodiments, A is —CH.

In some embodiments, R³ is —O-(unsubstituted or substituted C₁-C₆alkyl). In some embodiments, R³ is —OCH₃. In some embodiments, R³ is methoxy, ethoxy, n-propoxy, isopropoxy, n-butoxy, iso-butoxy, sec-butoxy, or tert-butoxy.

In some embodiments, R⁴ is hydrogen.

In some embodiments, R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²; or R⁶ and R⁷ are taken together with the intervening carbon atoms connecting R⁶ to R⁷ to form

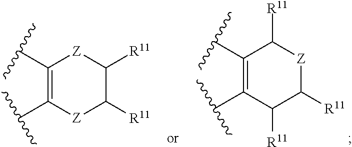

each Z is independently —C(R¹¹)₂, —N(R¹²)—, or —O—; each R¹¹ is independently hydrogen, halogen, oxo, unsubstituted or substituted C₁-C₆alkyl, unsubstituted or substituted C₁-C₆fluoroalkyl, —CN, —OH, —OR²⁰, —N(R²¹)₂, or —NR²¹C(═O)R²¹; each R¹³ is independently hydrogen, halogen, unsubstituted or substituted C₁-C₆alkyl, —CN, —OH, —OR²⁰, —CO₂R²¹, —C(═O)N(R²¹)₂, —N(R²¹)₂, or —NR²¹C(═O)R²¹; p is 0, 1, or 2.

In some embodiments, R⁶ and R⁷ are taken together with the intervening carbon atoms connecting R⁶ to R⁷ to form a ring B that is monocyclic C₃-C₈ aryl, wherein Ring B is unsubstituted or substituted with 1 or 2 R¹¹, or 0 or 1 R¹². In some embodiments, R⁶ and R⁷ are taken together with the intervening carbon atoms connecting R⁶ to R⁷ to form a ring B that is phenyl, wherein Ring B is unsubstituted or substituted with 1 or 2 R¹¹, or 0 or 1 R¹².

In some embodiments, R⁶ and R⁷ are taken together with the intervening carbon atoms connecting R⁶ to R⁷ to form a ring B that is monocyclic C₃-C₈ heteroaryl, wherein Ring B is unsubstituted or substituted with 1 or 2 R¹¹, or 0 or 1 R¹². In some embodiments, R⁶ and R⁷ are taken together with the intervening carbon atoms connecting R⁶ to R⁷ to form a ring B that is furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, or pyridazinyl, wherein Ring B is unsubstituted or substituted with 1 or 2 R¹¹, or 0 or 1 R¹².

In some embodiments,

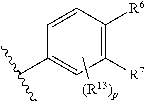

is

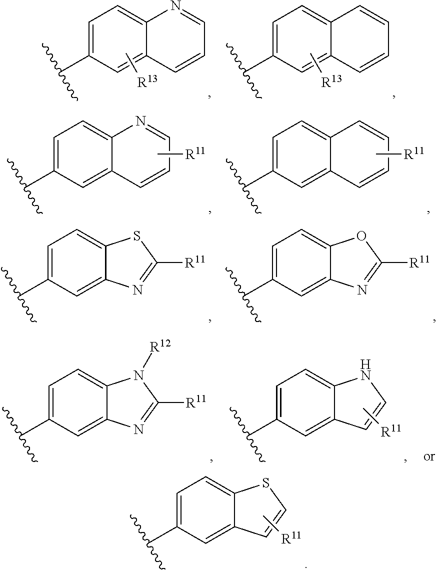

In some embodiments, R¹¹ is hydrogen, halogen, oxo, unsubstituted or substituted C₁-C₆alkyl, unsubstituted or substituted C₁-C₆fluoroalkyl, —CN, —OH, —OR²⁰, —N(R²¹)₂, or —NR²¹C(═O)R²¹. In some embodiments, R¹¹ is hydrogen, halogen, oxo, —OH, unsubstituted or substituted C₁-C₆alkyl, unsubstituted or substituted C₁-C₆fluoroalkyl, or unsubstituted or substituted C₁-C₆alkoxy. In some embodiments, R¹¹ is hydrogen, —F, —Br, —C₁, oxo, —OH, methyl, ethyl, propyl, iso-propyl, n-butyl, iso-butyl, tert-butyl, trifluoromethyl, difluoromethyl, fluoromethyl, trifluoroethyl, difluoroethyl, fluoroethyl, methoxy, ethoxy, n-propoxy, isopropoxy, n-butoxy, iso-butoxy, sec-butoxy, or tert-butoxy. In some embodiments, R¹¹ is hydrogen, —F, oxo, —OH, methyl, or methoxy.

In some embodiments, R¹² is hydrogen or unsubstituted or substituted C₁-C₆alkyl. In some embodiments, R¹² is hydrogen. In some embodiments, R¹² is unsubstituted or substituted C₁-C₆alkyl. In some embodiments, R¹² is methyl.

In some embodiments, R¹³ is hydrogen, halogen, or unsubstituted or substituted C₁-C₆alkyl. In some embodiments, R¹³ is hydrogen. In some embodiments, R¹³ is —F, —Cl, —Br, or —I. In some embodiments, R¹³ is methyl, ethyl, propyl, iso-propyl, n-butyl, iso-butyl, or tert-butyl.

In some embodiments, R⁶ and R⁷ are taken together with the intervening carbon atoms connecting R⁶ to R⁷ to form a ring B that is monocyclic C₃-C₈cycloalkyl or monocyclic C₃-C₈heterocycloalkyl, wherein ring B is unsubstituted or substituted with 1 or 2 R¹¹, or 0 or 1 R¹².

In some embodiments,

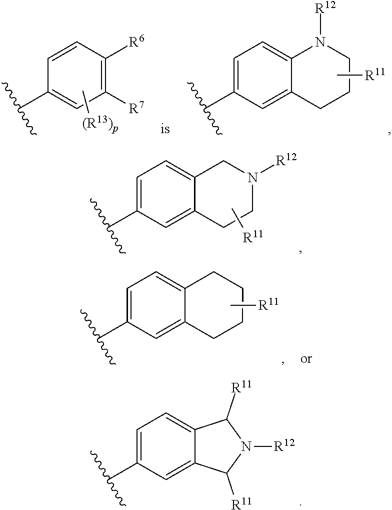

In another aspect described herein is a compound of Formula (IV):

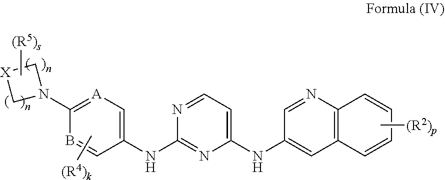

or a pharmaceutically acceptable salt or solvate thereof, wherein:
   each n is independently 1, 2, or 3;
   each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   s is 0, 1, 2, 3, or 4;
   X is —C(R⁸)₂—, —N(R⁹)—, —O—, —S—, —S(═O)—, or —SO₂—;
   each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
   or both R⁸ are taken together with the carbon to which they are attached to form a unsubstituted or substituted C₃-C₈cycloalkyl, unsubstituted or substituted C₃-C₈heterocycloalkyl;
   R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
   B is C(R³) or N;
   R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²;
   each R⁴ is independently hydrogen, halogen, unsubstituted or substituted C₁-C₆alkyl, or unsubstituted or substituted C₁-C₆alkoxy;
   k is 0, 1, or 2;
   A is C(R⁴) or N;
   each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OH, —OR²⁰, —C(═O)R²¹, —CO₂R²¹, —N(R²¹)₂, —SR²¹, —S(═O)R²⁰, or —SO₂R²;
p is 0, 1, 2, 3, or 4;
wherein
   when

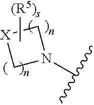

is

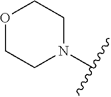 [SMILES: *N1CCOCC1]

and p is 0, at least one of R³ or R⁴ is —O-(substituted C₁-C₆alkyl); or
when

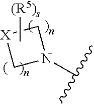

is

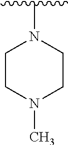 [SMILES: *N1CCN(C)CC1]

and p is 0, at least one of R³ or R⁴ is —O-(unsubstituted or substituted C₁-C₆alkyl);
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In some embodiments, each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —N(R²¹)₂, or —C(═O)N(R²¹)₂. In some embodiments, each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, or —N(R²¹)₂. In some embodiments, each R² is independently hydrogen, halogen, unsubstituted or substituted C₁-C₆alkyl, unsubstituted or substituted C₁-C₆fluoroalkyl, unsubstituted or substituted C₁-C₆alkoxy, —OH, or —C(═O)N(R²¹)₂. In some embodiments, each R² is independently hydrogen, halogen, unsubstituted or substituted C₁-C₆alkyl, unsubstituted or substituted C₁-C₆alkoxy, —OH, or —C(═O)N(H)₂. In some embodiments, each R² is independently hydrogen or halogen. In some embodiments, each R² is independently hydrogen, —F, —Cl, or —Br. In some embodiments, each R² is independently hydrogen or unsubstituted or substituted C₁-C₆alkoxy. In some embodiments, each R² is independently hydrogen or methoxy. In some embodiments, each R² is hydrogen.

In some embodiments, A is C(R⁴) and B is N; or A is N and B is C(R³). In some embodiments, A is C-(unsubstituted or substituted C₁-C₆alkoxy) and B is N; or A is N and B is C-(unsubstituted or substituted C₁-C₆alkoxy). In some embodiments, A is C(H) and B is N; or A is N and B is C(H). In some embodiments, A is N. In some embodiments, A is C(H). In some embodiments, A is C(OCH₃). In some embodiments, B is N. In some embodiments, B is C(H). In some embodiments, B is C(OCH₃). In some embodiments, B is C(OCH₂CH₂N(CH₃)₂).

In some embodiments, each n is independently 1 or 2; each R⁵ is independently hydrogen, halogen, unsubstituted or substituted C₁-C₆alkyl, unsubstituted or substituted C₁-C₆fluoroalkyl, —CN, OH, —OR²⁰, or N(R²¹)₂; and X is —C(R⁸)₂—, —N(R⁹)—, or —O—.

In some embodiments, each n is 1. In some embodiments, each n is 2.

In some embodiments, each R⁴ is independently hydrogen, halogen, unsubstituted or substituted C₁-C₆alkyl, or unsubstituted or substituted C₁-C₆alkoxy. In some embodiments, each R⁴ is independently hydrogen or unsubstituted or substituted C₁-C₆alkoxy. In some embodiments, each R⁴ is independently hydrogen, methoxy, ethoxy, or substituted ethoxy.

In some embodiments, each R⁵ is independently hydrogen, halogen, unsubstituted or substituted C₁-C₆alkyl, unsubstituted or substituted C₁-C₆fluoroalkyl, —CN, OH, —OR²⁰, or N(R²¹)₂. In some embodiments, each R⁵ is independently hydrogen, unsubstituted or substituted C₁-C₆alkyl, unsubstituted or substituted C₁-C₆fluoroalkyl, —OH, or N(R²¹)₂. In some embodiments, each R⁵ is independently hydrogen, methyl, ethyl, propyl, iso-propyl, n-butyl, iso-butyl, tert-butyl, trifluoromethyl, difluoromethyl, fluoromethyl, trifluoroethyl, difluoroethyl, fluoroethyl, —OH, or —N(CH₃)₂. In some embodiments, each R⁵ is independently hydrogen, methyl, trifluoromethyl, difluoromethyl, or —N(CH₃)₂.

In some embodiments, R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C₂R²¹, or —C(═O)N(R²¹)₂. In some embodiments, R⁹ is hydrogen, unsubstituted or substituted C₁-C₆alkyl, unsubstituted or substituted C₃-C₈cycloalkyl, or unsubstituted or substituted C₃-C₈heterocycloalkyl. In some embodiments, R⁹ is hydrogen. In some embodiments, R⁹ is methyl, ethyl, propyl, iso-propyl, n-butyl, iso-butyl, or tert-butyl. In some embodiments, R⁹ is methyl. In some embodiments, R⁹ is methyl substituted with —CN. In some embodiments, R⁹ is C₃-C₆heterocycloalkyl. In some embodiments, R⁹ is oxetanyl.

In some embodiments, X is —C(R⁸)₂—, —N(R⁹)—, or —O—; each R⁸ is independently hydrogen, C₁-C₆alkyl, unsubstituted or substituted C₃-C₈cycloalkyl, unsubstituted or substituted C₃-C₈heterocycloalkyl, —OH, —N(R²¹)₂; or both R⁸ are taken together with the carbon to which they are attached to form a unsubstituted or substituted C₃-C₈cycloalkyl, unsubstituted or substituted C₃-C₈heterocycloalkyl; and R⁹ is hydrogen, unsubstituted or substituted C₁-C₆alkyl, C₃-C₈cycloalkyl, —C₂R²¹, or —C(═O)N(R²¹)₂.

In some embodiments,

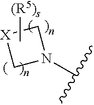

is

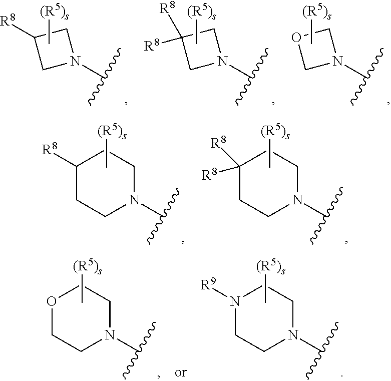

In some embodiments,

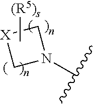

is

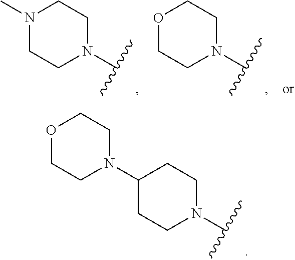

In another aspect described herein is a compound of Formula (V):

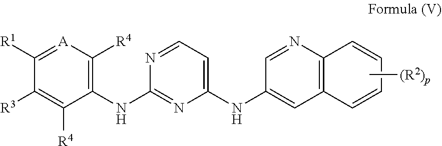

or a pharmaceutically acceptable salt or solvate thereof, wherein:
   R¹ and R³ are taken together with the intervening carbon atoms connecting R¹ to R³ to form a ring A that is

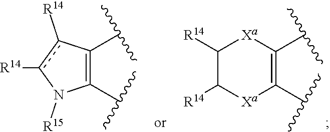

wherein [symbol] indicates a single or double bond;
each R¹⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, substituted or unsubstituted C₁-C₆alkyl-N(R²¹)₂, oxo, —CN, —OH, —OR²⁰, —C(═O)R²¹, —CO₂R²¹, —C(═O)N(R²¹)₂, —N(R¹)₂, —NR²¹C(═O)R²¹, —NR²¹CO₂R²¹, —SR²¹, —S(═O)R²⁰, —SO₂R²¹, or —SO₂N(R²¹)₂;
R¹⁵ is -substituted or unsubstituted C₁-C₆alkyl, -substituted or unsubstituted C₁-C₆alkyl-N(R²¹)₂, —C(═O)-substituted or unsubstituted C₁-C₆alkyl-N(R²¹)₂, -substituted or unsubstituted C₁-C₆alkyl-carbocycle, or heterocycle;
each X^{a} is independently —C(R¹⁶)₂—, —N(R¹⁷)—, —O—, or —S—;
each R¹⁶ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹⁷ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —C(═O)R²¹, —CO₂R²¹, or —C(═O)N(R²¹)₂;
each R⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
A is C(R⁴) or N;
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²;
p is 0, 1, 2, 3, or 4;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

In some embodiments, each R² is independently hydrogen, halogen, unsubstituted or substituted C₁-C₆alkyl, or unsubstituted or substituted C₁-C₆fluoroalkyl. In some embodiments, each R² is independently hydrogen, —F, —Cl, —Br, —I, halogen. In some embodiments, each R² is independently hydrogen, —F, —Cl, —Br, —I, methyl, ethyl, propyl, iso-propyl, n-butyl, iso-butyl, tert-butyl, trifluoromethyl, difluoromethyl, fluoromethyl, trifluoroethyl, difluoroethyl, or fluoroethyl. In some embodiments, each R² is hydrogen. In some embodiments, each R² is independently hydrogen, difluoromethyl, —F, —Cl, or —Br.

In some embodiments, each R¹⁴ is independently hydrogen, halogen, unsubstituted or substituted C₁-C₆alkyl, unsubstituted or substituted C₁-C₆fluoroalkyl, -(unsubstituted or substituted C₁-C₆alkyl)-N(R²)₂, oxo, or —CN; R¹⁵ is unsubstituted or substituted C₁-C₆alkyl, -(unsubstituted or substituted C₁-C₆alkyl)-N(R²¹)₂, —C(═O)-(unsubstituted or substituted C₁-C₆alkyl)-N(R²¹)₂, -(unsubstituted or substituted C₁-C₆alkyl)-carbocycle, or unsubstituted or substituted heterocycle; and each X^{a} is independently —N(R¹⁷)— or —O—.

In some embodiments, R¹ and R³ are taken together with the intervening carbon atoms connecting R¹ to R³ to form a ring A that is

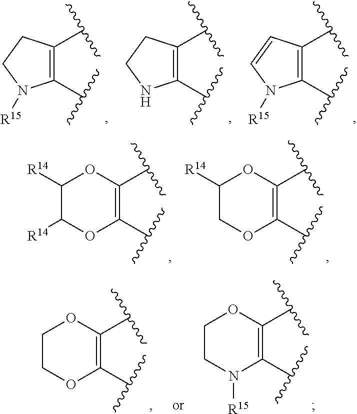

each R¹⁴ is independently halogen, unsubstituted or substituted C₁-C₆alkyl, unsubstituted or substituted C₁-C₆fluoroalkyl, -(unsubstituted or substituted C₁-C₆alkyl)-N(R²¹)₂, or oxo; and R¹⁵ is -(unsubstituted or substituted C₁-C₆alkyl)-N(R²¹)₂, —C(═O)-(unsubstituted or substituted C₁-C₆alkyl)-N(R²¹)₂, -(unsubstituted or substituted C₁-C₆alkyl)-(unsubstituted or substituted C₃-C₈carbocycle), or unsubstituted or substituted C₃-C₈heterocycle.

In some embodiments, R¹ and R³ are taken together with the intervening carbon atoms connecting R¹ to R³ to form a ring A that is

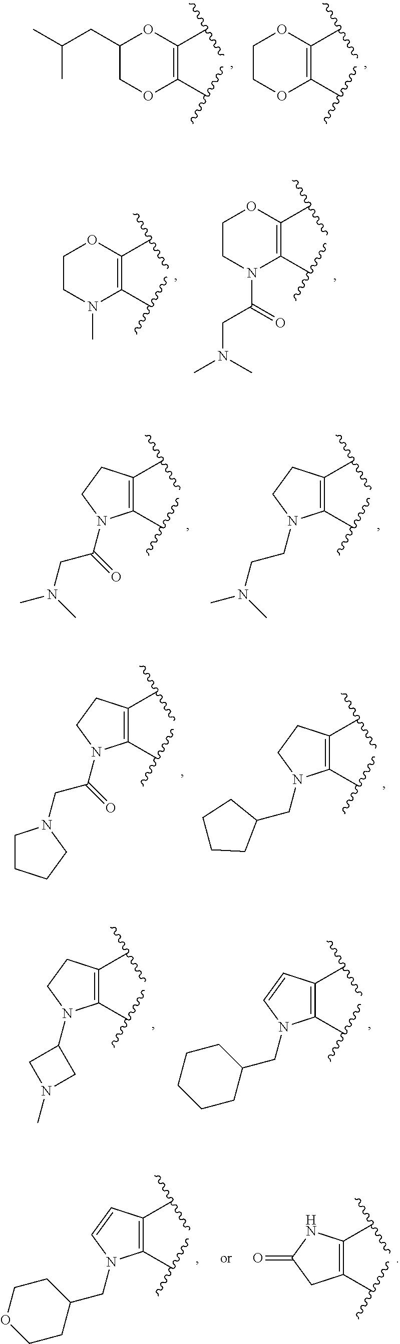

In some embodiments, each R¹⁴ is independently hydrogen or —CH₂—N(CH₃)₂; and R¹⁵ is —CH₂CH₂—N(CH₃)₂, —C(═O)—CH₂—N(CH₃)₂, —CH₂—(C₃-C₈ monocyclic carbocycle), or C₃-C₈heterocycle.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

That is, any combination of the groups described above for the various variables is contemplated herein. Throughout the specification, groups and substituents thereof are chosen by one skilled in the field to provide stable moieties and compounds.

Exemplary compounds of the invention include the compounds described in the following Tables:

TABLE 1

| Cmpd | | |
|---|---|---|
| # | Structure | Name |
| 11 | 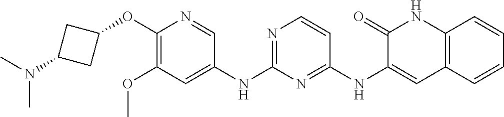 [SMILES: COc1cc(Nc2nccc(Nc3cc4ccccc4[nH]c3=O)n2)cnc1O[C@H]1C[C@@H](N(C)C)C1] | 3-(2-{5-methoxy-6-[(1s,3s)-3- (dimethylamino)cyclobutoxy]-3- pyridylamino}-4- pyrimidinylamino)-2(1H)- quinolinone |
| 45 | 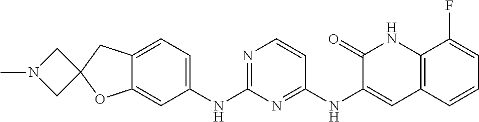 [SMILES: CN1CC2(Cc3ccc(Nc4nccc(Nc5cc6cccc(F)c6[nH]c5=O)n4)cc3O2)C1] | 8-fluoro-3-{2-(1-methyl-3′H- spiro[azetidine-3,2′-[1]benzofuran]- 6′-ylamino)-4-pyrimidinylamino}- 1,2-dihydro-2-quinolinone |
| 109 | 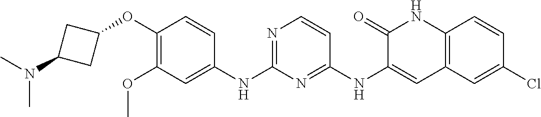 [SMILES: COc1cc(Nc2nccc(Nc3cc4cc(Cl)ccc4[nH]c3=O)n2)ccc1O[C@H]1C[C@H](N(C)C)C1] | 6-chloro-3-(2-{3-methoxy-4- [(1r,3r)-3- (dimethylamino)cyclobutoxy]phenyl- amino}-4-pyrimidinylamino)-1,2- dihydro-2-quinolinone |
| 110 | 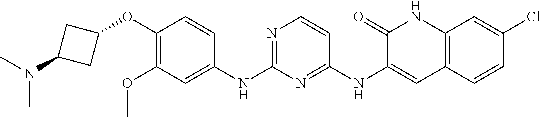 [SMILES: COc1cc(Nc2nccc(Nc3cc4ccc(Cl)cc4[nH]c3=O)n2)ccc1O[C@H]1C[C@H](N(C)C)C1] | 7-chloro-3-(2-{3-methoxy-4- [(1r,3r)-3- (dimethylamino)cyclobutoxy]phenyl- amino}-4-pyrimidinylamino)-1,2- dihydro-2-quinolinone |
| 111 | 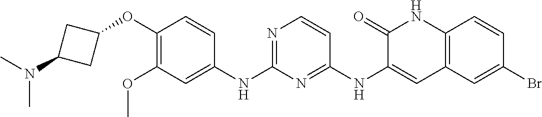 [SMILES: COc1cc(Nc2nccc(Nc3cc4cc(Br)ccc4[nH]c3=O)n2)ccc1O[C@H]1C[C@H](N(C)C)C1] | 4-(8-chloro-3-quinolylamino)-2-{3- methoxy-4-[(1s,4s)-4- (dimethylamino)cyclohexyloxy] phenylamino}pyrimidine |
| 112 | 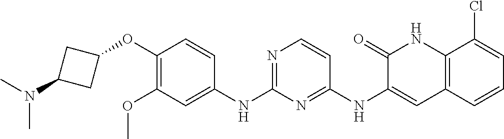 [SMILES: COc1cc(Nc2nccc(Nc3cc4cccc(Cl)c4[nH]c3=O)n2)ccc1O[C@H]1C[C@H](N(C)C)C1] | 8-chloro-3-(2-{3-methoxy-4- [(1r,3r)-3- (dimethylamino)cyclobutoxy]phenyl- amino}-4-pyrimidinylamino)-1,2- dihydro-2-quinolinone |
| 117 | 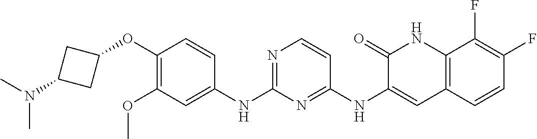 [SMILES: COc1cc(Nc2nccc(Nc3cc4ccc(F)c(F)c4[nH]c3=O)n2)ccc1O[C@H]1C[C@@H](N(C)C)C1] | 7,8-difluoro-3-(2-{3-methoxy-4- [(1s,3s)-3- (dimethylamino)cyclobutoxy]phenyl- amino}-4-pyrimidinylamino)-1,2- dihydro-2-quinolinone |
| 119 | 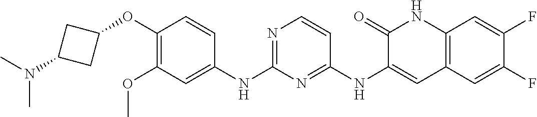 [SMILES: COc1cc(Nc2nccc(Nc3cc4cc(F)c(F)cc4[nH]c3=O)n2)ccc1O[C@H]1C[C@@H](N(C)C)C1] | 6,7-difluoro-3-(2-{3-methoxy-4- [(1s,3s)-3- (dimethylamino)cyclobutoxy]phenyl- amino}-4-pyrimidinylamino)-1,2- dihydro-2-quinolinone |
| 120 | 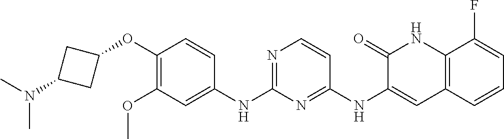 [SMILES: COc1cc(Nc2nccc(Nc3cc4cccc(F)c4[nH]c3=O)n2)ccc1O[C@H]1C[C@@H](N(C)C)C1] | 8-fluoro-3-(2-{3-methoxy-4- [(1s,3s)-3- (dimethylamino)cyclobutoxy]phenyl- amino}-4-pyrimidinylamino)-1,2- dihydro-2-quinolinone |
| 121 | 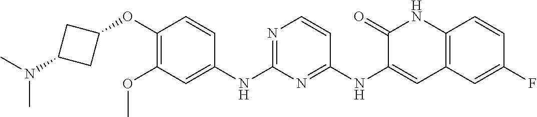 [SMILES: COc1cc(Nc2nccc(Nc3cc4cc(F)ccc4[nH]c3=O)n2)ccc1O[C@H]1C[C@@H](N(C)C)C1] | 6-fluoro-3-(2-{3-methoxy-4- [(1s,3s)-3- (dimethylamino)cyclobutoxy]phenyl- amino}-4-pyrimidinylamino)-1,2- dihydro-2-quinolinone |
| 122 | 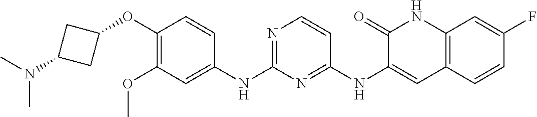 [SMILES: COc1cc(Nc2nccc(Nc3cc4ccc(F)cc4[nH]c3=O)n2)ccc1O[C@H]1C[C@@H](N(C)C)C1] | 7-fluoro-3-(2-{3-methoxy-4- [(1s,3s)-3- (dimethylamino)cyclobutoxy]phenyl- amino}-4-pyrimidinylamino)-1,2- dihydro-2-quinolinone |
| 123 | 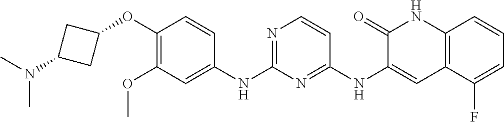 [SMILES: COc1cc(Nc2nccc(Nc3cc4c(F)cccc4[nH]c3=O)n2)ccc1O[C@H]1C[C@@H](N(C)C)C1] | 5-fluoro-3-(2-{3-methoxy-4- [(1s,3s)-3- (dimethylamino)cyclobutoxy]phenyl- amino}-4-pyrimidinylamino)-1,2- dihydro-2-quinolinone |
| 151 | 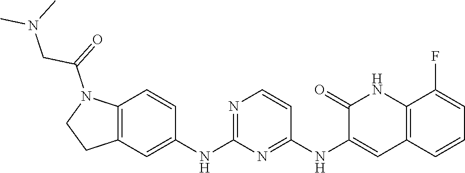 [SMILES: CN(C)CC(=O)N1CCc2cc(Nc3nccc(Nc4cc5cccc(F)c5[nH]c4=O)n3)ccc21] | 3-(2-{1-[2-(dimethylamino)acetyl]- 5-indolinylamino}-4- pyrimidinylamino)-8-fluoro-1,2- dihydro-2-quinolinone |
| 159 | 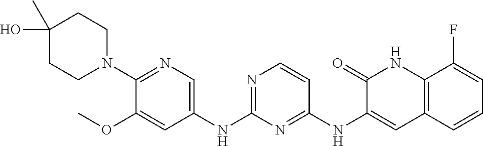 [SMILES: COc1cc(Nc2nccc(Nc3cc4cccc(F)c4[nH]c3=O)n2)cnc1N1CCC(C)(O)CC1] | 8-fluoro-3-{2-[6-(4-hydroxy-4- methyl-1-piperidyl)-5-methoxy-3- pyridylamino]-4- pyrimidinylamino}-1,2-dihydro-2- quinolinone |
| 160 | 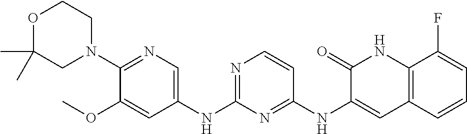 [SMILES: COc1cc(Nc2nccc(Nc3cc4cccc(F)c4[nH]c3=O)n2)cnc1N1CCOC(C)(C)C1] | 3-{2-[6-(2,2-dimethyl-4- morpholinyl)-5-methoxy-3- pyridylamino]-4- pyrimidinylamino}-8-fluoro-1,2- dihydro-2-quinolinone |
| 164 | 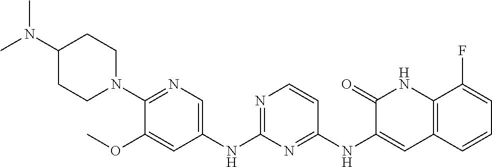 [SMILES: COc1cc(Nc2nccc(Nc3cc4cccc(F)c4[nH]c3=O)n2)cnc1N1CCC(N(C)C)CC1] | 3-(2-{6-[4-(dimethylamino)-1- piperidyl]-5-methoxy-3- pyridylamino}-4- pyrimidinylamino)-8-fluoro-1,2- dihydro-2-quinolinone |
| 165 | 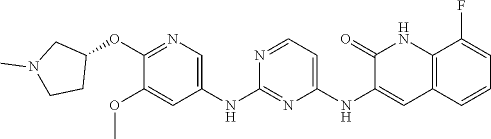 [SMILES: COc1cc(Nc2nccc(Nc3cc4cccc(F)c4[nH]c3=O)n2)cnc1O[C@@H]1CCN(C)C1] | 3-(2-{6-[(R)-1-methyl-3- pyrrolidinyloxy]-5-methoxy-3- pyridylamino}-4- pyrimidinylamino)-8-fluoro-1,2- dihydro-2-quinolinone |
| 166 | 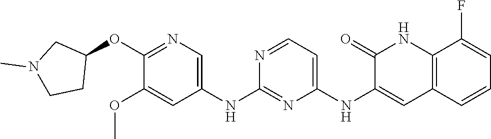 [SMILES: COc1cc(Nc2nccc(Nc3cc4cccc(F)c4[nH]c3=O)n2)cnc1O[C@H]1CCN(C)C1] | 3-(2-{6-[(S)-1-methyl-3- pyrrolidinyloxy]-5-methoxy-3- pyridylamino}-4- pyrimidinylamino)-8-fluoro-1,2- dihydro-2-quinolinone |
| 167 | 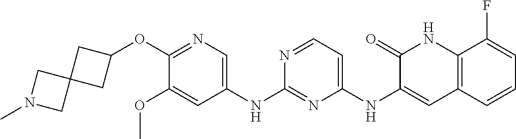 [SMILES: COc1cc(Nc2nccc(Nc3cc4cccc(F)c4[nH]c3=O)n2)cnc1OC1CC2(C1)CN(C)C2] | 8-fluoro-3-(2-{5-methoxy-6-(2- methyl-2-aza-6- spiro[3.3]heptyloxy)-3- pyridylamino}-4- pyrimidinylamino)-1,2-dihydro-2- quinolinone |
| 174 | 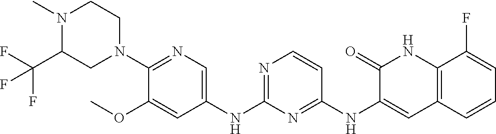 [SMILES: COc1cc(Nc2nccc(Nc3cc4cccc(F)c4[nH]c3=O)n2)cnc1N1CCN(C)C(C(F)(F)F)C1] | 8-fluoro-3-(2-{5-methoxy-6-[4- methyl-3-(trifluoromethyl)-1- piperazinyl]-3-pyridylamino}-4- pyrimidinylamino)-1,2-dihydro-2- quinolinone |
| 175 | 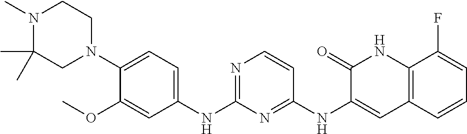 [SMILES: COc1cc(Nc2nccc(Nc3cc4cccc(F)c4[nH]c3=O)n2)ccc1N1CCN(C)C(C)(C)C1] | 3-{2-[4-(3,3-dimethyl-4-methyl-1- piperazinyl)-3-anisidino]-4- pyrimidinylamino}-8-fluoro-1,2- dihydro-2-quinolinone |
| 197 | 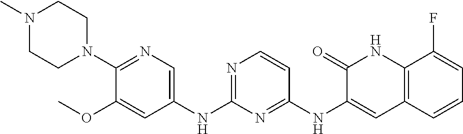 [SMILES: COc1cc(Nc2nccc(Nc3cc4cccc(F)c4[nH]c3=O)n2)cnc1N1CCN(C)CC1] | 8-fluoro-3-{2-[5-methoxy-6-(4- methyl-1-piperazinyl)-3- pyridylamino]-4- pyrimidinylamino}-1,2-dihydro-2- quinolinone |
| 205 | 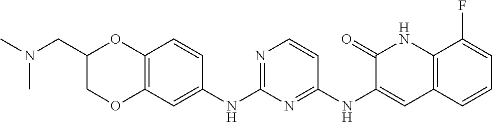 [SMILES: CN(C)CC1COc2cc(Nc3nccc(Nc4cc5cccc(F)c5[nH]c4=O)n3)ccc2O1] | 3-(2-{2-[(dimethylamino)methyl]- 2,3-dihydro-1,4-benzodioxin-6- ylamino}-4-pyrimidinylamino)-8- fluoro-1,2-dihydro-2-quinolinone |
| 208 | 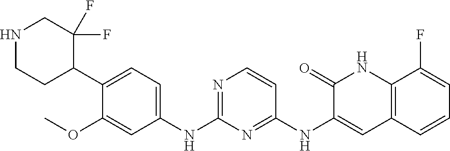 [SMILES: COc1cc(Nc2nccc(Nc3cc4cccc(F)c4[nH]c3=O)n2)ccc1C1CCNCC1(F)F] | 3-{2-[4-(3,3-difluoro-4-piperidyl)- 3-anisidino]-4-pyrimidinylamino}- 8-fluoro-1,2-dihydro-2-quinolinone |
| 211 | 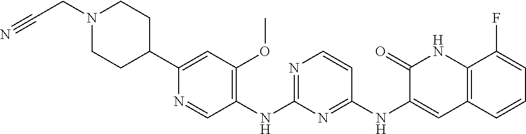 [SMILES: COc1cc(C2CCN(CC#N)CC2)ncc1Nc1nccc(Nc2cc3cccc(F)c3[nH]c2=O)n1] | (4-{5-[4-(8-fluoro-2-oxo-1,2- dihydro-3-quinolylamino)-2- pyrimidinylamino]-4-methoxy-2- pyridyl}-1-piperidyl)acetonitrile |
| 212 | 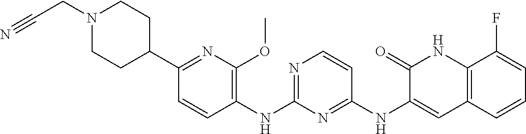 [SMILES: COc1nc(C2CCN(CC#N)CC2)ccc1Nc1nccc(Nc2cc3cccc(F)c3[nH]c2=O)n1] | (4-{5-[4-(8-fluoro-2-oxo-1,2- dihydro-3-quinolylamino)-2- pyrimidinylamino]-6-methoxy-2- pyridyl}-1-piperidyl)acetonitrile |
| 213 | 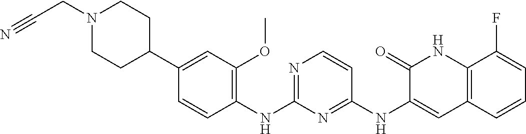 [SMILES: COc1cc(C2CCN(CC#N)CC2)ccc1Nc1nccc(Nc2cc3cccc(F)c3[nH]c2=O)n1] | (4-{4-[4-(8-fluoro-2-oxo-1,2- dihydro-3-quinolylamino)-2- pyrimidinylamino]-3- methoxyphenyl}-1- piperidyl)acetonitrile |
| 214 | 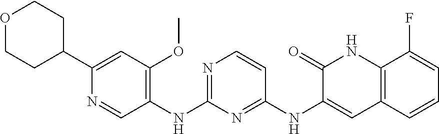 [SMILES: COc1cc(C2CCOCC2)ncc1Nc1nccc(Nc2cc3cccc(F)c3[nH]c2=O)n1] | 8-fluoro-3-{2-[4-methoxy-6- (tetrahydro-2H-pyran-4-yl)-3- pyridylamino]-4- pyrimidinylamino}-1,2-dihydro-2- quinolinone |
| 215 | 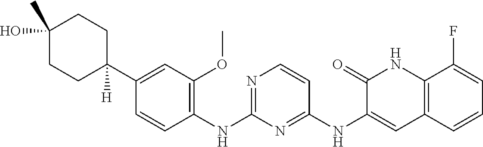 [SMILES: COc1cc([C@H]2CC[C@](C)(O)CC2)ccc1Nc1nccc(Nc2cc3cccc(F)c3[nH]c2=O)n1] | 8-fluoro-3-(2-{2-methoxy-4- [(1s,4s)-4-hydroxy-4- methylcyclohexyl]phenylamino}-4- pyrimidinylamino)-1,2-dihydro-2- quinolinone |
| 216 | 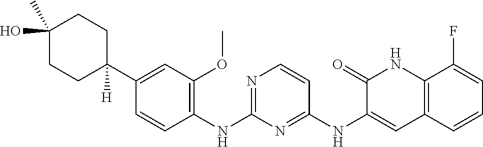 [SMILES: COc1cc([C@H]2CC[C@@](C)(O)CC2)ccc1Nc1nccc(Nc2cc3cccc(F)c3[nH]c2=O)n1] | 8-fluoro-3-(2-{2-methoxy-4- [(1r,4r)-4-hydroxy-4- methylcyclohexyl]phenylamino}-4- pyrimidinylamino)-1,2-dihydro-2- quinolinone |
| 217 | 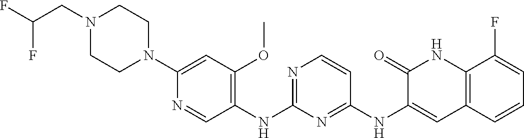 [SMILES: COc1cc(N2CCN(CC(F)F)CC2)ncc1Nc1nccc(Nc2cc3cccc(F)c3[nH]c2=O)n1] | 3-(2-{6-[4-(2,2-difluoroethyl)-1- piperazinyl]-4-methoxy-3- pyridylamino}-4- pyrimidinylamino)-8-fluoro-1,2- dihydro-2-quinolinone |
| 218 | 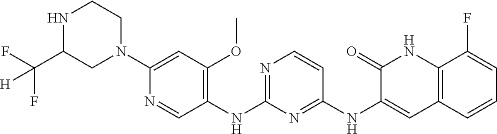 [SMILES: COc1cc(N2CCNC(C(F)F)C2)ncc1Nc1nccc(Nc2cc3cccc(F)c3[nH]c2=O)n1] | 3-(2-{6-[3-(difluoromethyl)-1- piperazinyl]-4-methoxy-3- pyridylamino}-4- pyrimidinylamino)-8-fluoro-1,2- dihydro-2-quinolinone |
| 219 | 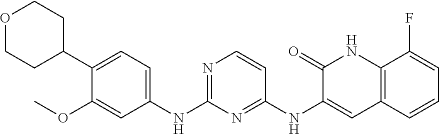 [SMILES: COc1cc(Nc2nccc(Nc3cc4cccc(F)c4[nH]c3=O)n2)ccc1C1CCOCC1] | 8-fluoro-3-{2-[3-methoxy-4- (tetrahydro-2H-pyran-4- yl)phenylamino]-4- pyrimidinylamino}-1,2-dihydro-2- quinolinone |
| 220 | 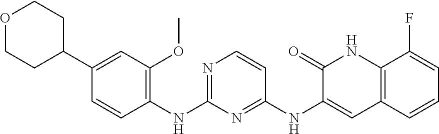 [SMILES: COc1cc(C2CCOCC2)ccc1Nc1nccc(Nc2cc3cccc(F)c3[nH]c2=O)n1] | 8-fluoro-3-{2-[2-methoxy-4- (tetrahydro-2H-pyran-4- yl)phenylamino]-4- pyrimidinylamino}-1,2-dihydro-2- quinolinone |
| 221 | 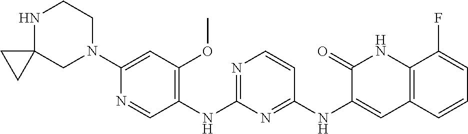 [SMILES: COc1cc(N2CCNC3(CC3)C2)ncc1Nc1nccc(Nc2cc3cccc(F)c3[nH]c2=O)n1] | 3-(2-{6-(4,7-diaza-7- spiro[2.5]octyl)-4-methoxy-3- pyridylamino}-4- pyrimidinylamino)-8-fluoro-1,2- dihydro-2-quinolinone |
| 222 | 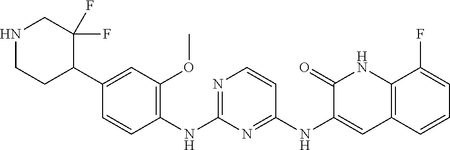 [SMILES: COc1cc(C2CCNCC2(F)F)ccc1Nc1nccc(Nc2cc3cccc(F)c3[nH]c2=O)n1] | 3-{2-[4-(3,3-difluoro-4-piperidyl)- 2-anisidino]-4-pyrimidinylamino}- 8-fluoro-1,2-dihydro-2-quinolinone |
| 223 | 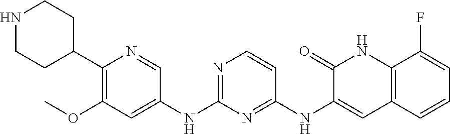 [SMILES: COc1cc(Nc2nccc(Nc3cc4cccc(F)c4[nH]c3=O)n2)cnc1C1CCNCC1] | 8-fluoro-3-{2-[5-methoxy-6-(4- piperidyl)-3-pyridylamino]-4- pyrimidinylamino}-1,2-dihydro-2- quinolinone |
| 224 | 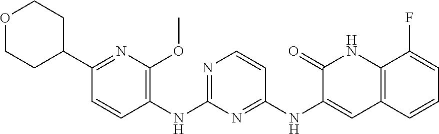 [SMILES: COc1nc(C2CCOCC2)ccc1Nc1nccc(Nc2cc3cccc(F)c3[nH]c2=O)n1] | 8-fluoro-3-{2-[2-methoxy-6- (tetrahydro-2H-pyran-4-yl)-3- pyridylamino]-4- pyrimidinylamino}-1,2-dihydro-2- quinolinone |
| 225 | 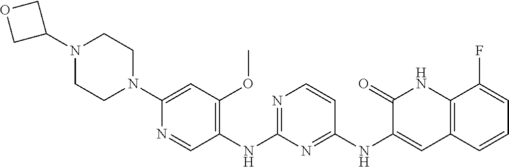 [SMILES: COc1cc(N2CCN(C3COC3)CC2)ncc1Nc1nccc(Nc2cc3cccc(F)c3[nH]c2=O)n1] | 8-fluoro-3-(2-{4-methoxy-6-[4-(3- oxetanyl)-1-piperazinyl]-3- pyridylamino}-4- pyrimidinylamino)-1,2-dihydro-2- quinolinone |
| 226 | 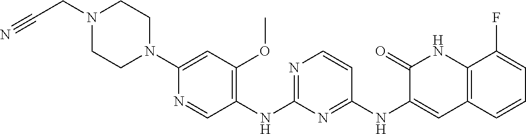 [SMILES: COc1cc(N2CCN(CC#N)CC2)ncc1Nc1nccc(Nc2cc3cccc(F)c3[nH]c2=O)n1] | (4-{5-[4-(8-fluoro-2-oxo-1,2- dihydro-3-quinolylamino)-2- pyrimidinylamino]-4-methoxy-2- pyridyl}-1-piperazinyl)acetonitrile |
| 227 | 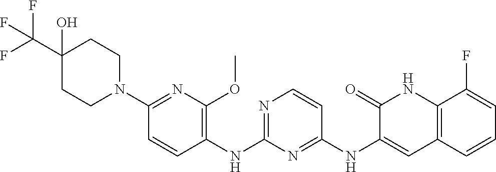 [SMILES: COc1nc(N2CCC(O)(C(F)(F)F)CC2)ccc1Nc1nccc(Nc2cc3cccc(F)c3[nH]c2=O)n1] | 8-fluoro-3-(2-{6-[4-hydroxy-4- (trifluoromethyl)-1-piperidyl]-2- methoxy-3-pyridylamino}-4- pyrimidinylamino)-1,2-dihydro-2- quinolinone |
| 228 | 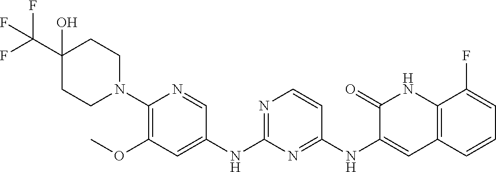 [SMILES: COc1cc(Nc2nccc(Nc3cc4cccc(F)c4[nH]c3=O)n2)cnc1N1CCC(O)(C(F)(F)F)CC1] | 8-fluoro-3-(2-{6-[4-hydroxy-4- (trifluoromethyl)-1-piperidyl]-5- methoxy-3-pyridylamino}-4- pyrimidinylamino)-1,2-dihydro-2- quinolinone |
| 229 | 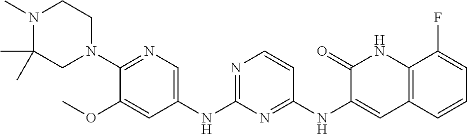 [SMILES: COc1cc(Nc2nccc(Nc3cc4cccc(F)c4[nH]c3=O)n2)cnc1N1CCN(C)C(C)(C)C1] | 3-{2-[6-(3,3-dimethyl-4-methyl-1- piperazinyl)-5-methoxy-3- pyridylamino]-4- pyrimidinylamino}-8-fluoro-1,2- dihydro-2-quinolinone |
| 230 | 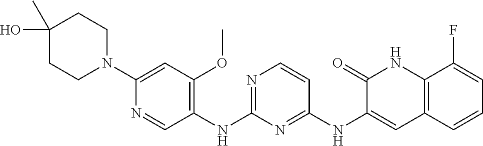 [SMILES: COc1cc(N2CCC(C)(O)CC2)ncc1Nc1nccc(Nc2cc3cccc(F)c3[nH]c2=O)n1] | 8-fluoro-3-{2-[6-(4-hydroxy-4- methyl-1-piperidyl)-4-methoxy-3- pyridylamino]-4- pyrimidinylamino}-1,2-dihydro-2- quinolinone |
| 231 | 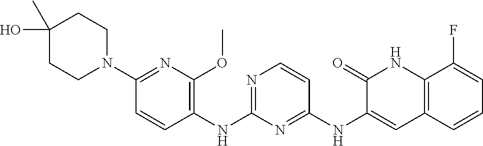 [SMILES: COc1nc(N2CCC(C)(O)CC2)ccc1Nc1nccc(Nc2cc3cccc(F)c3[nH]c2=O)n1] | 8-fluoro-3-{2-[6-(4-hydroxy-4- methyl-1-piperidyl)-2-methoxy-3- pyridylamino]-4- pyrimidinylamino}-1,2-dihydro-2- quinolinone |
| 232 | 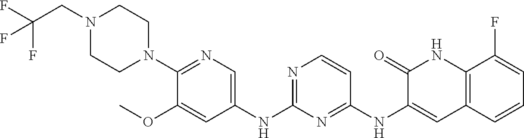 [SMILES: COc1cc(Nc2nccc(Nc3cc4cccc(F)c4[nH]c3=O)n2)cnc1N1CCN(CC(F)(F)F)CC1] | 8-fluoro-3-(2-{5-methoxy-6-[4- (2,2,2-trifluoroethyl)-1-piperazinyl]- 3-pyridylamino}-4- pyrimidinylamino)-1,2-dihydro-2- quinolinone |
| 233 | 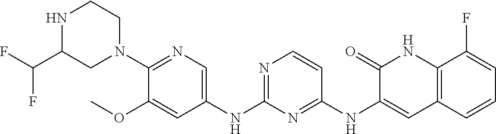 [SMILES: COc1cc(Nc2nccc(Nc3cc4cccc(F)c4[nH]c3=O)n2)cnc1N1CCNC(C(F)F)C1] | 3-(2-{6-[3-(difluoromethyl)-1- piperazinyl]-5-methoxy-3- pyridylamino}-4- pyrimidinylamino)-8-fluoro-1,2- dihydro-2-quinolinone |
| 234 | 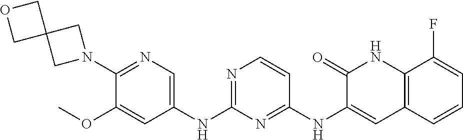 [SMILES: COc1cc(Nc2nccc(Nc3cc4cccc(F)c4[nH]c3=O)n2)cnc1N1CC2(COC2)C1] | 8-fluoro-3-(2-{5-methoxy-6-(2-oxa- 6-aza-6-spiro[3.3]heptyl)-3- pyridylamino}-4- pyrimidinylamino)-1,2-dihydro-2- quinolinone |
| 235 | 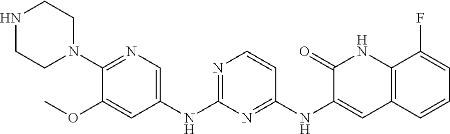 [SMILES: COc1cc(Nc2nccc(Nc3cc4cccc(F)c4[nH]c3=O)n2)cnc1N1CCNCC1] | 8-fluoro-3-{2-[5-methoxy-6-(1- piperazinyl)-3-pyridylamino]-4- pyrimidinylamino}-1,2-dihydro-2- quinolinone |
| 236 | 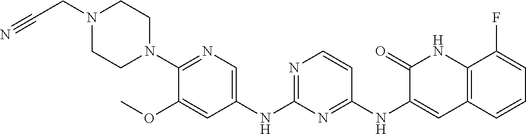 [SMILES: COc1cc(Nc2nccc(Nc3cc4cccc(F)c4[nH]c3=O)n2)cnc1N1CCN(CC#N)CC1] | (4-{5-[4-(8-fluoro-2-oxo-1,2- dihydro-3-quinolylamino)-2- pyrimidinylamino]-3-methoxy-2- pyridyl}-1-piperazinyl)acetonitrile |
| 237 | 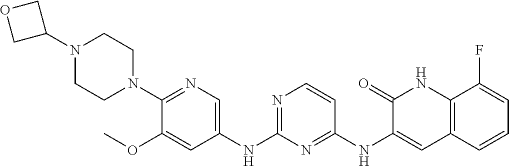 [SMILES: COc1cc(Nc2nccc(Nc3cc4cccc(F)c4[nH]c3=O)n2)cnc1N1CCN(C2COC2)CC1] | 8-fluoro-3-(2-{5-methoxy-6-[4-(3- oxetanyl)-1-piperazinyl]-3- pyridylamino}-4- pyrimidinylamino)-1,2-dihydro-2- quinolinone |
| 238 | 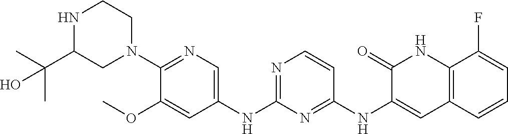 [SMILES: COc1cc(Nc2nccc(Nc3cc4cccc(F)c4[nH]c3=O)n2)cnc1N1CCNC(C(C)(C)O)C1] | 8-fluoro-3-(2-{6-[3-(1-hydroxy-1- methylethyl)-1-piperazinyl]-5- methoxy-3-pyridylamino}-4- pyrimidinylamino)-1,2-dihydro-2- quinolinone |
| 239 | 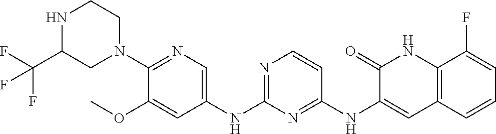 [SMILES: COc1cc(Nc2nccc(Nc3cc4cccc(F)c4[nH]c3=O)n2)cnc1N1CCNC(C(F)(F)F)C1] | 8-fluoro-3-(2-{5-methoxy-6-[3- (trifluoromethyl)-1-piperazinyl]-3- pyridylamino}-4- pyrimidinylamino)-1,2-dihydro-2- quinolinone |
| 240 | 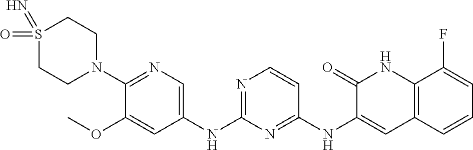 [SMILES: COc1cc(Nc2nccc(Nc3cc4cccc(F)c4[nH]c3=O)n2)cnc1N1CCS(=N)(=O)CC1] | 4-{5-[4-(8-fluoro-2-oxo-1,2- dihydro-3-quinolylamino)-2- pyrimidinylamino]-3-methoxy-2- pyridyl}-1-imino-1λ⁶,4-thiazinan-1- one |
| 241 | 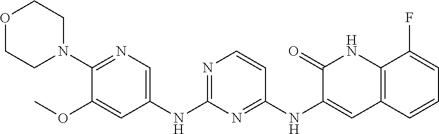 [SMILES: COc1cc(Nc2nccc(Nc3cc4cccc(F)c4[nH]c3=O)n2)cnc1N1CCOCC1] | 8-fluoro-3-[2-(5-methoxy-6- morpholino-3-pyridylamino)-4- pyrimidinylamino]-1,2-dihydro-2- quinolinone |
| 242 | 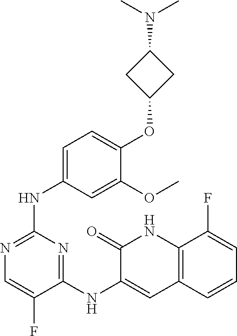 [SMILES: COc1cc(Nc2ncc(F)c(Nc3cc4cccc(F)c4[nH]c3=O)n2)ccc1O[C@H]1C[C@@H](N(C)C)C1] | 8-fluoro-3-(5-fluoro-2-{3-methoxy- 4-[(1s,3s)-3- (dimethylamino)cyclobutoxy]phenyl- amino}-4-pyrimidinylamino)-1,2- dihydro-2-quinolinone |
| 243 | 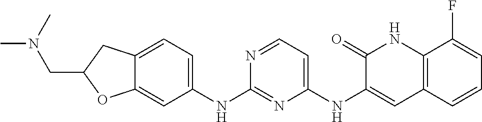 [SMILES: CN(C)CC1Cc2ccc(Nc3nccc(Nc4cc5cccc(F)c5[nH]c4=O)n3)cc2O1] | 3-(2-{2-[(dimethylamino)methyl]- 2,3-dihydro-1-benzofuran-6- ylamino}-4-pyrimidinylamino)-8- fluoro-1,2-dihydro-2-quinolinone |
| 244 | 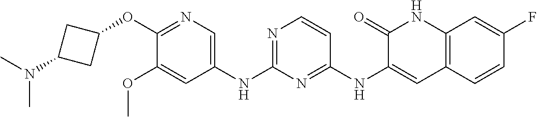 [SMILES: COc1cc(Nc2nccc(Nc3cc4ccc(F)cc4[nH]c3=O)n2)cnc1O[C@H]1C[C@@H](N(C)C)C1] | 7-fluoro-3-(2-{5-methoxy-6- [(1s,3s)-3- (dimethylamino)cyclobutoxy]-3- pyridylamino}-4- pyrimidinylamino)-1,2-dihydro-2- quinolinone |
| 245 | 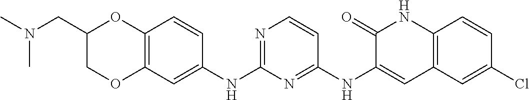 [SMILES: CN(C)CC1COc2cc(Nc3nccc(Nc4cc5cc(Cl)ccc5[nH]c4=O)n3)ccc2O1] | 6-chloro-3-(2-{2- [(dimethylamino)methyl]-2,3- dihydro-1,4-benzodioxin-6- ylamino}-4-pyrimidinylamino)-1,2- dihydro-2-quinolinone |
| 246 | 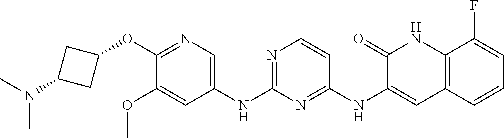 [SMILES: COc1cc(Nc2nccc(Nc3cc4cccc(F)c4[nH]c3=O)n2)cnc1O[C@H]1C[C@@H](N(C)C)C1] | 8-fluoro-3-(2-{5-methoxy-6- [(1s,3s)-3- (dimethylamino)cyclobutoxy]-3- pyridylamino}-4- pyrimidinylamino)-1,2-dihydro-2- quinolinone |
| 247 | 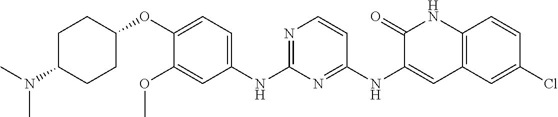 [SMILES: COc1cc(Nc2nccc(Nc3cc4cc(Cl)ccc4[nH]c3=O)n2)ccc1O[C@H]1CC[C@@H](N(C)C)CC1] | 6-chloro-3-(2-{3-methoxy-4- [(1s,4s)-4-(dimethylamino) cyclohexyloxy]phenyl- amino}-4-pyrimidinylamino)- 1,2-dihydro-2-quinolinone |
| 248 | 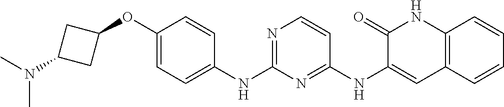 [SMILES: CN(C)[C@H]1C[C@H](Oc2ccc(Nc3nccc(Nc4cc5ccccc5[nH]c4=O)n3)cc2)C1] | 3-(2-{p-[(1r,3r)-3- (dimethylamino)cyclobutoxy] phenylamino}-4-pyrimidinyl- amino)-2(1H)-quinolinone |
| 249 | 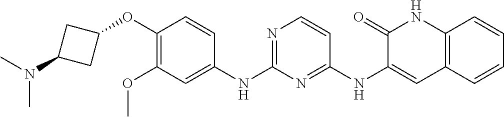 [SMILES: COc1cc(Nc2nccc(Nc3cc4ccccc4[nH]c3=O)n2)ccc1O[C@H]1C[C@H](N(C)C)C1] | 3-(2-{3-methoxy-4-[(1r,3r)-3- (dimethylamino)cyclobutoxy]phenyl- amino}-4-pyrimidinylamino)- 2(1H)-quinolinone |
| 250 | 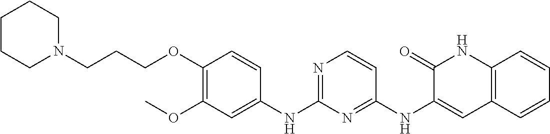 [SMILES: COc1cc(Nc2nccc(Nc3cc4ccccc4[nH]c3=O)n2)ccc1OCCCN1CCCCC1] | 3-{2-[3-methoxy-4-(3- piperidinopropoxy)phenylamino]-4- pyrimidinylamino}-2(1H)- quinolinone |
| 251 | 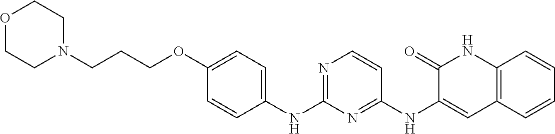 [SMILES: O=c1[nH]c2ccccc2cc1Nc1ccnc(Nc2ccc(OCCCN3CCOCC3)cc2)n1] | 3-{2-[p-(3- morpholinopropoxy)phenylamino]- 4-pyrimidinylamino}-2(1H)- quinolinone |

In some embodiments, the compound is a pharmaceutically acceptable salt of a compound described in Table 1.

TABLE 2

| Cmpd | | |
|---|---|---|
| # | Structure | Name |
| 7 | 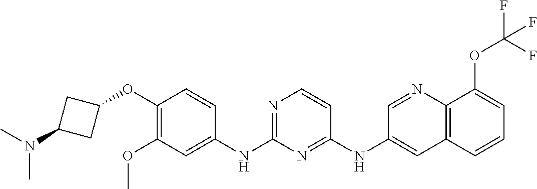 [SMILES: COc1cc(Nc2nccc(Nc3cnc4c(OC(F)(F)F)cccc4c3)n2)ccc1O[C@H]1C[C@H](N(C)C)C1] | 2-{3-methoxy-4-[(1r,3r)-3- (dimethylamino)cyclobutoxy] phenylamino}-4-(8- trifluoromethoxy-3- quinolylamino)pyrimidine |
| 8 | 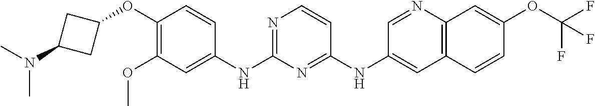 [SMILES: COc1cc(Nc2nccc(Nc3cnc4cc(OC(F)(F)F)ccc4c3)n2)ccc1O[C@H]1C[C@H](N(C)C)C1] | 2-{3-methoxy-4-[(1r,3r)-3- (dimethylamino)cyclobutoxy] phenylamino}-4-(7- trifluoromethoxy-3- quinolylamino)pyrimidine |
| 9 | 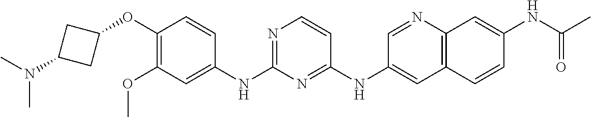 [SMILES: COc1cc(Nc2nccc(Nc3cnc4cc(NC(C)=O)ccc4c3)n2)ccc1O[C@H]1C[C@@H](N(C)C)C1] | N-[3-(2-{3-methoxy-4- [(1s,3s)-3- (dimethylamino)cyclobutoxy] phenylamino}-4- pyrimidinylamino)-7- quinolyl]acetamide |
| 10 | 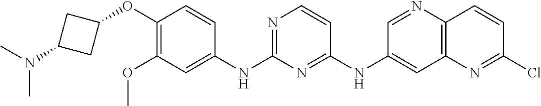 [SMILES: COc1cc(Nc2nccc(Nc3cnc4ccc(Cl)nc4c3)n2)ccc1O[C@H]1C[C@@H](N(C)C)C1] | 4-(6-chloro-1,5-diaza-3- naphthylamino)-2-{3- methoxy-4-[(1s,3s)-3- (dimethylamino)cyclobutoxy] phenylamino}pyrimidine |
| 12 | 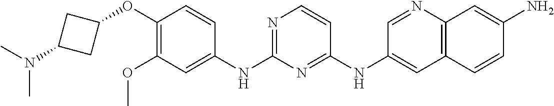 [SMILES: COc1cc(Nc2nccc(Nc3cnc4cc(N)ccc4c3)n2)ccc1O[C@H]1C[C@@H](N(C)C)C1] | 4-(7-amino-3-quinolylamino)- 2-{3-methoxy-4-[(1s,3s)-3- (dimethylamino)cyclobutoxy] phenylamino}pyrimidine |
| 15 | 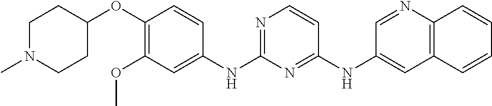 [SMILES: COc1cc(Nc2nccc(Nc3cnc4ccccc4c3)n2)ccc1OC1CCN(C)CC1] | 2-[3-methoxy-4-(1-methyl-4- piperidyloxy)phenylamino]-4- (3-quinolylamino)pyrimidine |
| 16 | 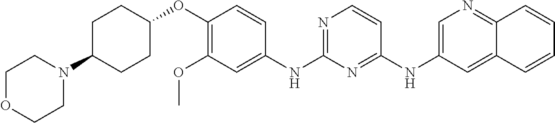 [SMILES: COc1cc(Nc2nccc(Nc3cnc4ccccc4c3)n2)ccc1O[C@H]1CC[C@H](N2CCOCC2)CC1] | 2-{3-methoxy-4-[(1r,4r)-4- morpholinocyclohexyloxy] phenylamino}-4-(3- quinolylamino)pyrimidine |
| 17 | 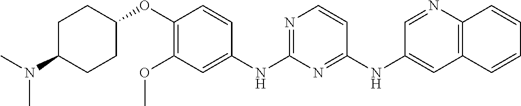 [SMILES: COc1cc(Nc2nccc(Nc3cnc4ccccc4c3)n2)ccc1O[C@H]1CC[C@H](N(C)C)CC1] | 2-{3-methoxy-4-[(1r,4r)-4- (dimethylamino)cyclohexyloxy] phenylamino}-4-(3- quinolylamino)pyrimidine |
| 18 | 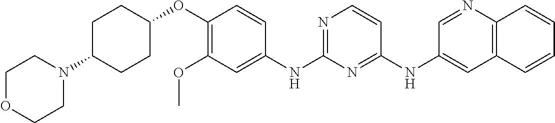 [SMILES: COc1cc(Nc2nccc(Nc3cnc4ccccc4c3)n2)ccc1O[C@H]1CC[C@@H](N2CCOCC2)CC1] | 2-{3-methoxy-4-[(1s,4s)-4- morpholinocyclohexyloxy] phenylamino}-4-(3- quinolylamino)pyrimidine |
| 19 | 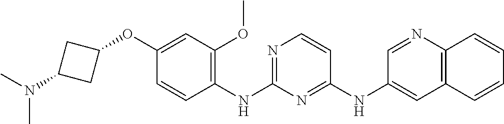 [SMILES: COc1cc(O[C@H]2C[C@@H](N(C)C)C2)ccc1Nc1nccc(Nc2cnc3ccccc3c2)n1] | 2-{2-methoxy-4-[(1s,3s)-3- (dimethylamino)cyclobutoxy] phenylamino}-4-(3- quinolylamino)pyrimidine |
| 21 | 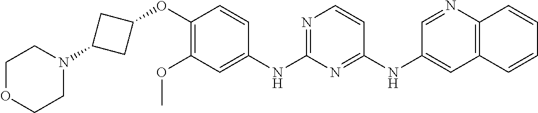 [SMILES: COc1cc(Nc2nccc(Nc3cnc4ccccc4c3)n2)ccc1O[C@H]1C[C@@H](N2CCOCC2)C1] | 2-{3-methoxy-4-[(1s,3s)-3- morpholinocyclobutoxy] phenylamino}-4-(3- quinolylamino)pyrimidine |
| 22 | 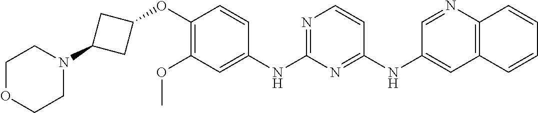 [SMILES: COc1cc(Nc2nccc(Nc3cnc4ccccc4c3)n2)ccc1O[C@H]1C[C@H](N2CCOCC2)C1] | 2-{3-methoxy-4-[(1r,3r)-3- morpholinocyclobutoxy] phenylamino}-4-(3- quinolylamino)pyrimidine |
| 23 | 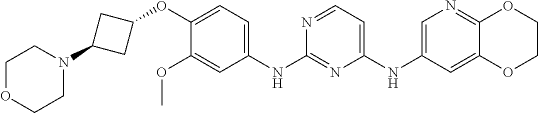 [SMILES: COc1cc(Nc2nccc(Nc3cnc4c(c3)OCCO4)n2)ccc1O[C@H]1C[C@H](N2CCOCC2)C1] | 4-(2,3-dihydro-1,4-dioxa-5- aza-7-naphthylamino)-2-{3- methoxy-4-[(1r,3r)-3- morpholinocyclobutoxy] phenylamino}pyrimidine |
| 24 | 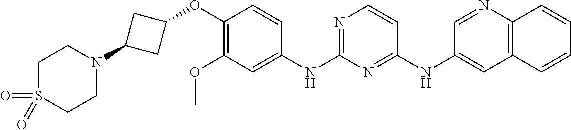 [SMILES: COc1cc(Nc2nccc(Nc3cnc4ccccc4c3)n2)ccc1O[C@H]1C[C@H](N2CCS(=O)(=O)CC2)C1] | 4-[(1r,3r)-3-{2-methoxy-4-[4- (3-quinolylamino)-2- pyrimidinylaminophenoxy} cyclobutyl]-1λ⁶,4-thiazinane- 1,1-dione |
| 25 | 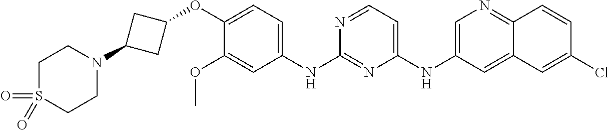 [SMILES: COc1cc(Nc2nccc(Nc3cnc4ccc(Cl)cc4c3)n2)ccc1O[C@H]1C[C@H](N2CCS(=O)(=O)CC2)C1] | 4-[(1r,3r)-3-{4-[4-(6-chloro-3- quinolylamino)-2- pyrimidinylamino]-2- methoxyphenoxy}cyclobutyl]- 1λ⁶,4-thiazinane-1,1-dione |
| 26 | 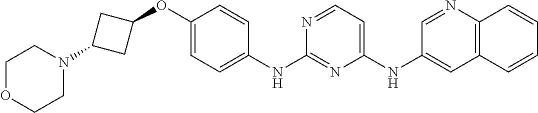 [SMILES: c1ccc2ncc(Nc3ccnc(Nc4ccc(O[C@H]5C[C@H](N6CCOCC6)C5)cc4)n3)cc2c1] | 4-(3-quinolylamino)-2- {p-[(1r,3r)-3- morpholinocyclobutoxy] phenylamino}pyrimidine |
| 27 | 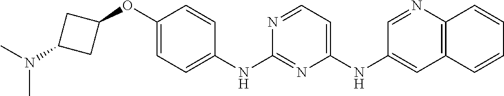 [SMILES: CN(C)[C@H]1C[C@H](Oc2ccc(Nc3nccc(Nc4cnc5ccccc5c4)n3)cc2)C1] | 4-(3-quinolylamino)-2-{p- [(1r,3r)-3- (dimethylamino)cyclobutoxy] phenylamino}pyrimidine |
| 28 | 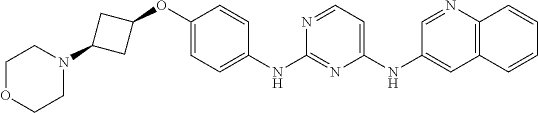 [SMILES: c1ccc2ncc(Nc3ccnc(Nc4ccc(O[C@H]5C[C@@H](N6CCOCC6)C5)cc4)n3)cc2c1] | 4-(3-quinolylamino)-2-{p- [(1s,3s)-3- morpholinocyclobutoxy] phenylamino}pyrimidine |
| 29 | 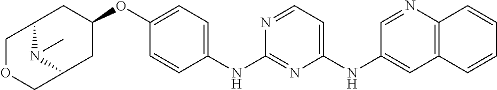 [SMILES: CN1[C@@H]2COC[C@H]1C[C@@H](Oc1ccc(Nc3nccc(Nc4cnc5ccccc5c4)n3)cc1)C2] | 2-(p-{(1R,5S,7r)-9-methyl-3- oxa-9-azabicyclo[3.3.1]non-7- yloxy}phenylamino)-4-(3- quinolylamino)pyrimidine |
| 30 | 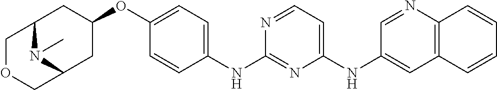 [SMILES: CN1[C@@H]2COC[C@H]1C[C@H](Oc1ccc(Nc3nccc(Nc4cnc5ccccc5c4)n3)cc1)C2] | 2-(p-{(1R,5S,7s)-9-methyl-3- oxa-9-azabicyclo[3.3.1]non-7- yloxy}phenylamino)-4-(3- quinolylamino)pyrimidine |
| 32 | 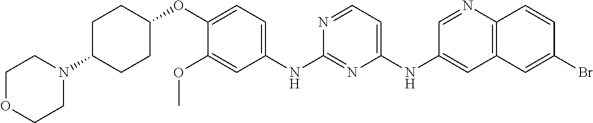 [SMILES: COc1cc(Nc2nccc(Nc3cnc4ccc(Br)cc4c3)n2)ccc1O[C@H]1CC[C@@H](N2CCOCC2)CC1] | 4-(6-bromo-3-quinolylamino)- 2-{3-methoxy-4-[(1s,4s)-4- morpholinocyclohexyloxy] phenylamino}pyrimidine |
| 33 | 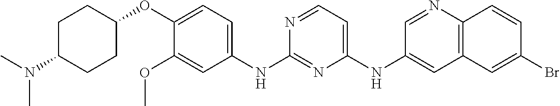 [SMILES: COc1cc(Nc2nccc(Nc3cnc4ccc(Br)cc4c3)n2)ccc1O[C@H]1CC[C@@H](N(C)C)CC1] | 4-(6-bromo-3-quinolylamino)- 2-{3-methoxy-4-[(1s,4s)-4- (dimethylamino)cyclohexyloxy] phenylamino}pyrimidine |
| 34 | 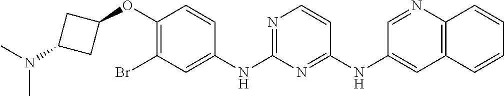 [SMILES: CN(C)[C@H]1C[C@H](Oc2ccc(Nc3nccc(Nc4cnc5ccccc5c4)n3)cc2Br)C1] | 2-{3-bromo-4-[(1r,3r)-3- (dimethylamino)cyclobutoxy] phenylamino}-4-(3- quinolylamino)pyrimidine |
| 35 | 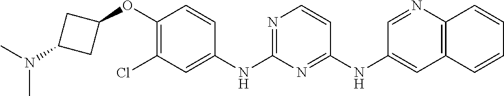 [SMILES: CN(C)[C@H]1C[C@H](Oc2ccc(Nc3nccc(Nc4cnc5ccccc5c4)n3)cc2Cl)C1] | 2-{3-chloro-4-[(1r,3r)-3- (dimethylamino)cyclobutoxy] phenylamino}-4-(3- quinolylamino)pyrimidine |
| 36 | 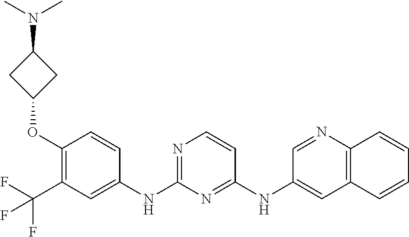 [SMILES: CN(C)[C@H]1C[C@H](Oc2ccc(Nc3nccc(Nc4cnc5ccccc5c4)n3)cc2C(F)(F)F)C1] | 4-(3-quinolylamino)-2-{4- [(1r,3r)-3- (dimethylamino)cyclobutoxy]- 3- (trifluoromethyl)phenylamino} pyrimidine |
| 37 | 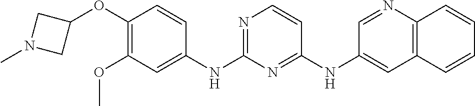 [SMILES: COc1cc(Nc2nccc(Nc3cnc4ccccc4c3)n2)ccc1OC1CN(C)C1] | 2-[3-methoxy-4-(1-methyl-3- azetidinyloxy)phenylamino]- 4-(3- quinolylamino)pyrimidine |
| 38 | 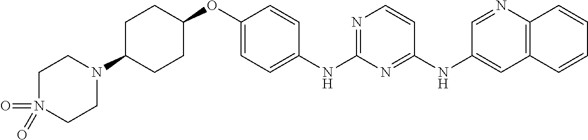 | 4-[(1s,4s)-4-{p-[4-(3- quinolylamino)-2- pyrimidinylamino]phenoxy} cyclohexyl]-126,4-thiazinane- 1,1-dione |
| 39 | 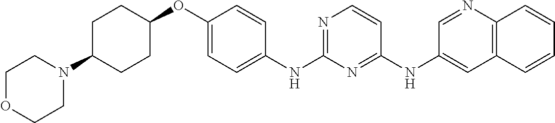 [SMILES: c1ccc2ncc(Nc3ccnc(Nc4ccc(O[C@H]5CC[C@@H](N6CCOCC6)CC5)cc4)n3)cc2c1] | 4-(3-quinolylamino)-2-{p- [(1s, 4s)-4- morpholinocyclohexyloxy] phenylamino}pyrimidine |
| 40 | 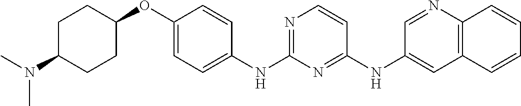 [SMILES: CN(C)[C@H]1CC[C@@H](Oc2ccc(Nc3nccc(Nc4cnc5ccccc5c4)n3)cc2)CC1] | 4-(3-quinolylamino)-2-{p- [(1s,4s)-4- (dimethylamino)cyclohexyloxy] phenylamino}pyrimidine |
| 41 | 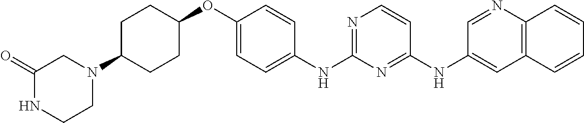 [SMILES: O=C1CN([C@H]2CC[C@@H](Oc3ccc(Nc4nccc(Nc5cnc6ccccc6c5)n4)cc3)CC2)CCN1] | 4-[(1s,4s)-4-{p-[4-(3- quinolylamino)-2- pyrimidinylaminophenoxy} cyclohexyl]-2-piperazinone |
| 42 | 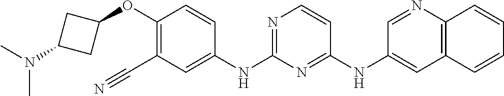 [SMILES: CN(C)[C@H]1C[C@H](Oc2ccc(Nc3nccc(Nc4cnc5ccccc5c4)n3)cc2C#N)C1] | 5-[4-(3-quinolylamino)-2- pyrimidinylamino]-2-[(1r,3r)- 3- (dimethylamino)cyclobutoxy] benzonitrile |
| 43 | 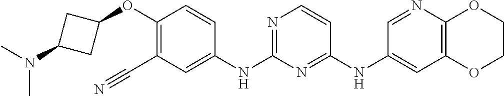 [SMILES: CN(C)[C@H]1C[C@@H](Oc2ccc(Nc3nccc(Nc4cnc5c(c4)OCCO5)n3)cc2C#N)C1] | 5-[4-(2,3-dihydro-1,4-dioxa- 5-aza-7-naphthylamino)-2- pyrimidinylamino]-2-[(1s,3s)- 3- (dimethylamino)cyclobutoxy] benzonitrile |
| 44 | 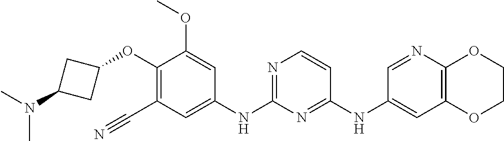 [SMILES: COc1cc(Nc2nccc(Nc3cnc4c(c3)OCCO4)n2)cc(C#N)c1O[C@H]1C[C@H](N(C)C)C1] | 5-[4-(2,3-dihydro-1,4-dioxa- 5-aza-7-naphthylamino)-2- pyrimidinylamino]-3- methoxy-2-[(1r,3r)-3- (dimethylamino)cyclobutoxy] benzonitrile |
| 46 | 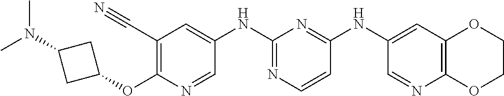 [SMILES: CN(C)[C@H]1C[C@@H](Oc2ncc(Nc3nccc(Nc4cnc5c(c4)OCCO5)n3)cc2C#N)C1] | 5-[4-(2,3-dihydro-1,4-dioxa- 5-aza-7-naphthylamino)-2- pyrimidinylamino]-2-[(1s,3s)- 3- (dimethylamino)cyclobutoxy] nicotinonitrile |
| 47 | 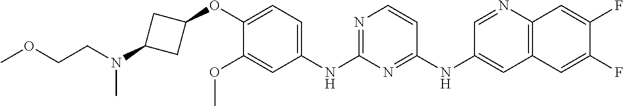 [SMILES: COCCN(C)[C@H]1C[C@@H](Oc2ccc(Nc3nccc(Nc4cnc5cc(F)c(F)cc5c4)n3)cc2OC)C1] | 4-(6,7-difluoro-3- quinolylamino)-2-(3- methoxy-4-{(1s,3s)-3-[(2- methoxyethyl)-N- methylamino]cyclobutoxy} phenylamino)pyrimidine |
| 75 | 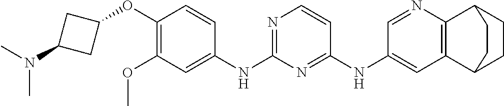 [SMILES: COc1cc(Nc2nccc(Nc3cnc4c(c3)C3CCC4CC3)n2)ccc1O[C@H]1C[C@H](N(C)C)C1] | 4-{3- azatricyclo[6.2.2.02,7]dodeca- 2(7),3,5-trien-5-ylamino}-2- {3-methoxy-4-[(1r,3r)-3- (dimethylamino)cyclobutoxy] phenylamino}pyrimidine |
| 76 | 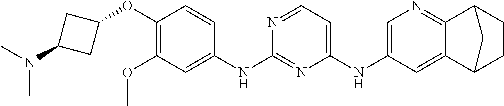 [SMILES: COc1cc(Nc2nccc(Nc3cnc4c(c3)C3CCC4C3)n2)ccc1O[C@H]1C[C@H](N(C)C)C1] | 4-(3- azatricyclo[6.2.1.02,7]undeca- 2,4,6-trien-5-ylamino)-2-{3- methoxy-4-[(1r,3r)-3- (dimethylamino)cyclobutoxy] phenylamino}pyrimidine |
| 77 | 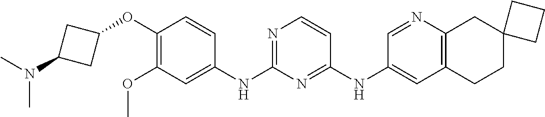 [SMILES: COc1cc(Nc2nccc(Nc3cnc4c(c3)CCC3(CCC3)C4)n2)ccc1O[C@H]1C[C@H](N(C)C)C1] | 4-(6′,8′-dihydro-5′H- spiro[cyclobutane-1,7′- quinolin]-3′-ylamino)-2-{3- methoxy-4-[(1r,3r)-3- (dimethylamino)cyclobutoxy] phenylamino}pyrimidine |
| 78 | 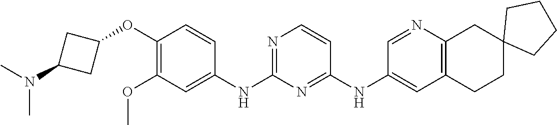 [SMILES: COc1cc(Nc2nccc(Nc3cnc4c(c3)CCC3(CCCC3)C4)n2)ccc1O[C@H]1C[C@H](N(C)C)C1] | 4-(6′,8′-dihydro-5′H- spiro[cyclopentane-1,7′- quinolin]-3′-ylamino)-2-{3- methoxy-4-[(1r,3r)-3- (dimethylamino)cyclobutoxy] phenylamino}pyrimidine |
| 79 | 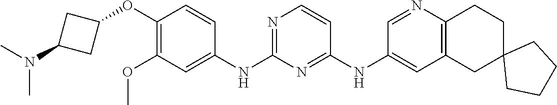 [SMILES: COc1cc(Nc2nccc(Nc3cnc4c(c3)CC3(CCCC3)CC4)n2)ccc1O[C@H]1C[C@H](N(C)C)C1] | 4-(7′,8′-dihydro-5′H- spiro[cyclopentane-1,6′- quinolin]-3′-ylamino)-2-{3- methoxy-4-[(1r,3r)-3- (dimethylamino)cyclobutoxy] phenylamino}pyrimidine |
| 80 | 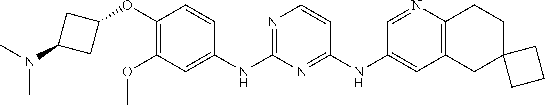 [SMILES: COc1cc(Nc2nccc(Nc3cnc4c(c3)CC3(CCC3)CC4)n2)ccc1O[C@H]1C[C@H](N(C)C)C1] | 4-(7′,8′-dihydro-5′H- spiro[cyclobutane-1,6′- quinolin]-3′-ylamino)-2-{3- methoxy-4-[(1r,3r)-3- (dimethylamino)cyclobutoxy] phenylamino}pyrimidine |
| 81 | 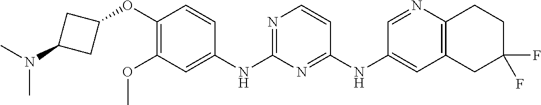 [SMILES: COc1cc(Nc2nccc(Nc3cnc4c(c3)CC(F)(F)CC4)n2)ccc1O[C@H]1C[C@H](N(C)C)C1] | 4-(6,6-difluoro-5,6,7,8- tetrahydro-3-quinolylamino)- 2-{3-methoxy-4-[(1r,3r)-3- (dimethylamino)cyclobutoxy] phenylamino}pyrimidine |
| 82 | 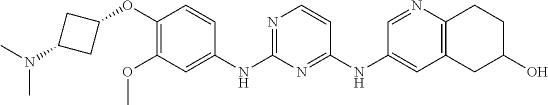 [SMILES: COc1cc(Nc2nccc(Nc3cnc4c(c3)CC(O)CC4)n2)ccc1O[C@H]1C[C@@H](N(C)C)C1] | 3-(2-{3-methoxy-4-[(1s,3s)-3- (dimethylamino)cyclobutoxy] phenylamino}-4- pyrimidinylamino)-5,6,7,8- tetrahydro-6-quinolinol |
| 83 | 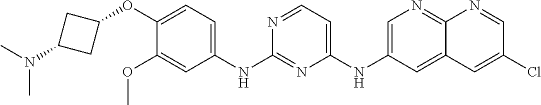 [SMILES: COc1cc(Nc2nccc(Nc3cnc4ncc(Cl)cc4c3)n2)ccc1O[C@H]1C[C@@H](N(C)C)C1] | 4-(6-chloro-1,8-diaza-3- naphthylamino)-2-{3- methoxy-4-[(1s,3s)-3- (dimethylamino)cyclobutoxy] phenylamino}pyrimidine |
| 84 | 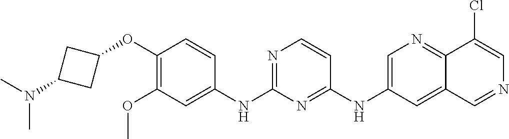 [SMILES: COc1cc(Nc2nccc(Nc3cnc4c(Cl)cncc4c3)n2)ccc1O[C@H]1C[C@@H](N(C)C)C1] | 4-(8-chloro-1,6-diaza-3- naphthylamino)-2-{3- methoxy-4-[(1s,3s)-3- (dimethylamino)cyclobutoxy] phenylamino}pyrimidine |
| 85 | 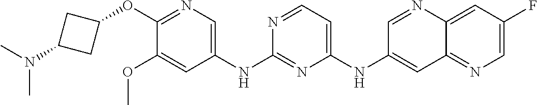 [SMILES: COc1cc(Nc2nccc(Nc3cnc4cc(F)cnc4c3)n2)cnc1O[C@H]1C[C@@H](N(C)C)C1] | 4-(7-fluoro-1,5-diaza-3- naphthylamino)-2-{5- methoxy-6-[(1s,3s)-3- (dimethylamino)cyclobutoxy]- 3-pyridylamino}pyrimidine |
| 86 | 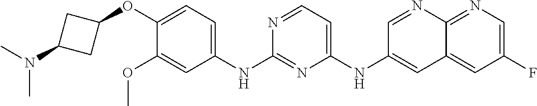 [SMILES: COc1cc(Nc2nccc(Nc3cnc4ncc(F)cc4c3)n2)ccc1O[C@H]1C[C@@H](N(C)C)C1] | 4-(6-fluoro-1,8-diaza-3- naphthylamino)-2-{3- methoxy-4-[(1s,3s)-3- (dimethylamino)cyclobutoxy] phenylamino}pyrimidine |
| 87 | 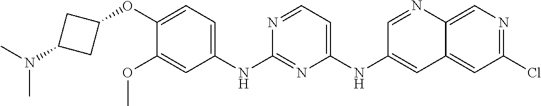 [SMILES: COc1cc(Nc2nccc(Nc3cnc4cnc(Cl)cc4c3)n2)ccc1O[C@H]1C[C@@H](N(C)C)C1] | 4-(6-chloro-1,7-diaza-3- naphthylamino)-2-{3- methoxy-4-[(1s,3s)-3- (dimethylamino)cyclobutoxy] phenylamino}pyrimidine |
| 88 | 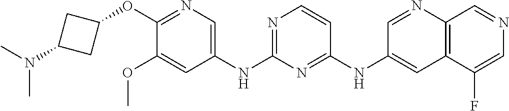 [SMILES: COc1cc(Nc2nccc(Nc3cnc4cncc(F)c4c3)n2)cnc1O[C@H]1C[C@@H](N(C)C)C1] | 4-(5-fluoro-1,7-diaza-3- naphthylamino)-2-{5- methoxy-6-[(1s,3s)-3- (dimethylamino)cyclobutoxy]- 3-pyridylamino}pyrimidine |
| 89 | 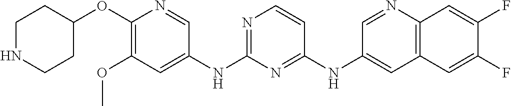 [SMILES: COc1cc(Nc2nccc(Nc3cnc4cc(F)c(F)cc4c3)n2)cnc1OC1CCNCC1] | 4-(6,7-difluoro-3- quinolylamino)-2-[5- methoxy-6-(4-piperidyloxy)- 3-pyridylamino]pyrimidine |
| 113 | 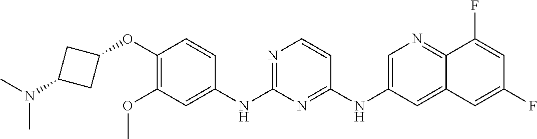 [SMILES: COc1cc(Nc2nccc(Nc3cnc4c(F)cc(F)cc4c3)n2)ccc1O[C@H]1C[C@@H](N(C)C)C1] | 4-(6,8-difluoro-3- quinolylamino)-2-{3- methoxy-4-[(1s,3s)-3- (dimethylamino)cyclobutoxy] phenylamino}pyrimidine |
| 114 | 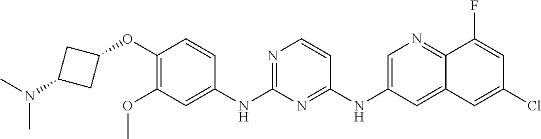 [SMILES: COc1cc(Nc2nccc(Nc3cnc4c(F)cc(Cl)cc4c3)n2)ccc1O[C@H]1C[C@@H](N(C)C)C1] | 4-(6-chloro-8-fluoro-3- quinolylamino)-2-{3- methoxy-4-[(1s,3s)-3- (dimethylamino)cyclobutoxy] phenylamino}pyrimidine |
| 115 | 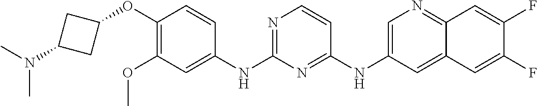 [SMILES: COc1cc(Nc2nccc(Nc3cnc4cc(F)c(F)cc4c3)n2)ccc1O[C@H]1C[C@@H](N(C)C)C1] | 4-(6,7-difluoro-3- quinolylamino)-2-{3- methoxy-4-[(1s,3s)-3- (dimethylamino)cyclobutoxy] phenylamino}pyrimidine |
| 116 | 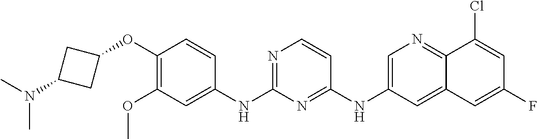 [SMILES: COc1cc(Nc2nccc(Nc3cnc4c(Cl)cc(F)cc4c3)n2)ccc1O[C@H]1C[C@@H](N(C)C)C1] | 4-(8-chloro-6-fluoro-3- quinolylamino)-2-{3- methoxy-4-[(1s,3s)-3- (dimethylamino)cyclobutoxy] phenylamino}pyrimidine |
| 118 | 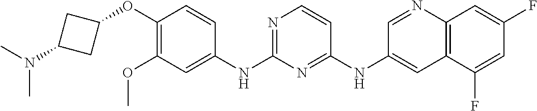 [SMILES: COc1cc(Nc2nccc(Nc3cnc4cc(F)cc(F)c4c3)n2)ccc1O[C@H]1C[C@@H](N(C)C)C1] | 4-(5,7-difluoro-3- quinolylamino)-2-{3- methoxy-4-[(1s,3s)-3- (dimethylamino)cyclobutoxy] phenylamino}pyrimidine |
| 124 | 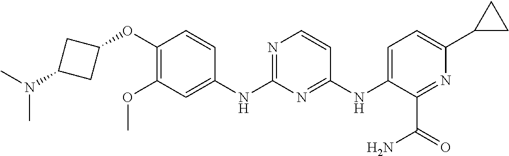 [SMILES: COc1cc(Nc2nccc(Nc3ccc(C4CC4)nc3C(N)=O)n2)ccc1O[C@H]1C[C@@H](N(C)C)C1] | 6-cyclopropyl-3-(2-{3- methoxy-4-[(1s,3s)-3- (dimethylamino)cyclobutoxy] phenylamino}-4- pyrimidinylamino)-2- pyridinecarboxamide |
| 125 | 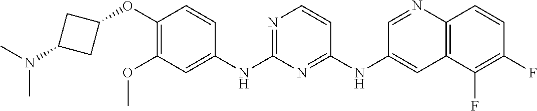 [SMILES: COc1cc(Nc2nccc(Nc3cnc4ccc(F)c(F)c4c3)n2)ccc1O[C@H]1C[C@@H](N(C)C)C1] | 4-(5,6-difluoro-3- quinolylamino)-2-{3- methoxy-4-[(1s,3s)-3- (dimethylamino)cyclobutoxy] phenylamino}pyrimidine |
| 128 | 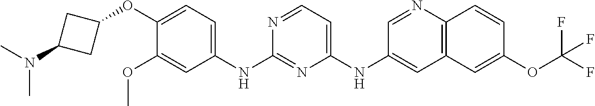 [SMILES: COc1cc(Nc2nccc(Nc3cnc4ccc(OC(F)(F)F)cc4c3)n2)ccc1O[C@H]1C[C@H](N(C)C)C1] | 2-{3-methoxy-4-[(1r,3r)-3- (dimethylamino)cyclobutoxy] phenylamino}-4-(6- trifluoromethoxy-3- quinolylamino)pyrimidine |
| 131 | 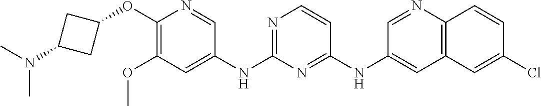 [SMILES: COc1cc(Nc2nccc(Nc3cnc4ccc(Cl)cc4c3)n2)cnc1O[C@H]1C[C@@H](N(C)C)C1] | 4-(6-chloro-3-quinolylamino)- 2-{5-methoxy-6-[(1s,3s)-3- (dimethylamino)cyclobutoxy]- 3-pyridylamino}pyrimidine |
| 132 | 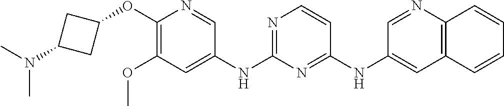 [SMILES: COc1cc(Nc2nccc(Nc3cnc4ccccc4c3)n2)cnc1O[C@H]1C[C@@H](N(C)C)C1] | 2-{5-methoxy-6-[(1s,3s)-3- (dimethylamino)cyclobutoxy]- 3-pyridylamino}-4-(3- quinolylamino)pyrimidine |
| 133 | 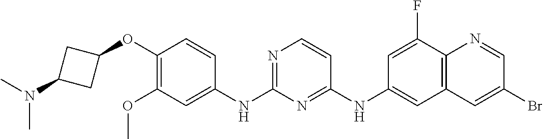 [SMILES: COc1cc(Nc2nccc(Nc3cc(F)c4ncc(Br)cc4c3)n2)ccc1O[C@H]1C[C@@H](N(C)C)C1] | 4-(3-bromo-8-fluoro-6- quinolylamino)-2-{3- methoxy-4-[(1s,3s)-3- (dimethylamino)cyclobutoxy] phenylamino}pyrimidine |
| 134 | 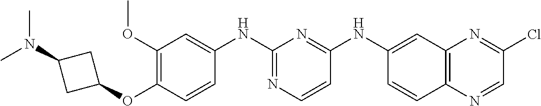 [SMILES: COc1cc(Nc2nccc(Nc3ccc4ncc(Cl)nc4c3)n2)ccc1O[C@H]1C[C@@H](N(C)C)C1] | 4-(3-chloro-6- quinoxalinylamino)-2-{3- methoxy-4-[(1s,3s)-3- (dimethylamino)cyclobutoxy] phenylamino}pyrimidine |
| 153 | 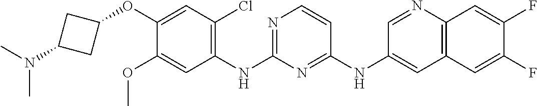 [SMILES: COc1cc(Nc2nccc(Nc3cnc4cc(F)c(F)cc4c3)n2)c(Cl)cc1O[C@H]1C[C@@H](N(C)C)C1] | 2-{2-chloro-5-methoxy-4- [(1s,3s)-3- (dimethylamino)cyclobutoxy] phenylamino}-4-(6,7- difluoro-3- quinolylamino)pyrimidine |
| 154 | 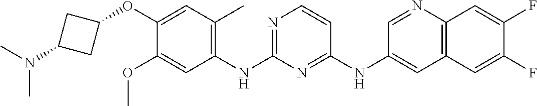 [SMILES: COc1cc(Nc2nccc(Nc3cnc4cc(F)c(F)cc4c3)n2)c(C)cc1O[C@H]1C[C@@H](N(C)C)C1] | 4-(6,7-difluoro-3- quinolylamino)-2-{5- methoxy-4-[(1s,3s)-3- (dimethylamino)cyclobutoxy]- 2-toluidino}pyrimidine |
| 162 | 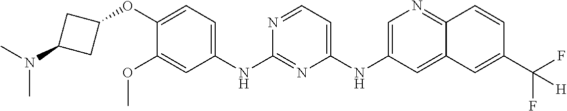 [SMILES: COc1cc(Nc2nccc(Nc3cnc4ccc(C(F)F)cc4c3)n2)ccc1O[C@H]1C[C@H](N(C)C)C1] | 4-[6-(difluoromethyl)-3- quinolylamino]-2-{3- methoxy-4-[(1r,3r)-3- (dimethylamino)cyclobutoxy] phenylamino}pyrimidine |
| 163 | 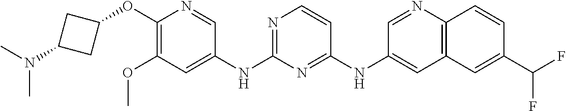 [SMILES: COc1cc(Nc2nccc(Nc3cnc4ccc(C(F)F)cc4c3)n2)cnc1O[C@H]1C[C@@H](N(C)C)C1] | 4-[6-(difluoromethyl)-3- quinolylamino]-2-{5- methoxy-6-[(1s,3s)-3- (dimethylamino)cyclobutoxy]- 3-pyridylamino}pyrimidine |
| 168 | 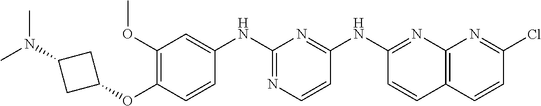 [SMILES: COc1cc(Nc2nccc(Nc3ccc4ccc(Cl)nc4n3)n2)ccc1O[C@H]1C[C@@H](N(C)C)C1] | 4-(7-chloro-1,8-diaza-2- naphthylamino)-2-{3- methoxy-4-[(1s,3s)-3- (dimethylamino)cyclobutoxy] phenylamino}pyrimidine |
| 172 | 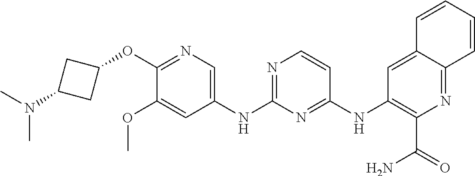 [SMILES: COc1cc(Nc2nccc(Nc3cc4ccccc4nc3C(N)=O)n2)cnc1O[C@H]1C[C@@H](N(C)C)C1] | 3-(2-{5-methoxy-6-[(1s,3s)-3- (dimethylamino)cyclobutoxy]- 3-pyridylamino}-4- pyrimidinylamino)-2- quinolinecarboxamide |
| 173 | 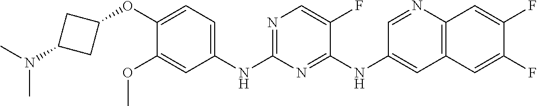 [SMILES: COc1cc(Nc2ncc(F)c(Nc3cnc4cc(F)c(F)cc4c3)n2)ccc1O[C@H]1C[C@@H](N(C)C)C1] | 4-(6,7-difluoro-3- quinolylamino)-5-fluoro-2-{3- methoxy-4-[(1s,3s)-3- (dimethylamino)cyclobutoxy] phenylamino}pyrimidine |
| 179 | 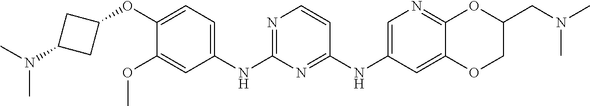 | 4-{3- [(dimethylamino)methyl]-2,3- dihydro-1,4-dioxa-5-aza-7- naphthylamino}-2-{3- methoxy-4-[(1s,3s)-3- (dimethylamino)cyclobutoxy] phenylamino}pyrimidine |
| 180 | 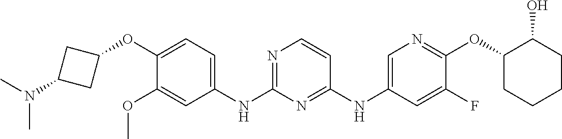 [SMILES: COc1cc(Nc2nccc(Nc3cnc(O[C@H]4CCCC[C@H]4O)c(F)c3)n2)ccc1O[C@H]1C[C@@H](N(C)C)C1] | (1R,2S)-2-[3-fluoro-5-(2-{3- methoxy-4-[(1s,3R)-3- (dimethylamino)cyclobutoxy] phenylamino}-4- pyrimidinylamino)-2- pyridyloxy]cyclohexanol |
| 190 | 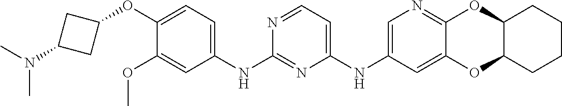 [SMILES: COc1cc(Nc2nccc(Nc3cnc4c(c3)O[C@@H]3CCCC[C@@H]3O4)n2)ccc1O[C@H]1C[C@@H](N(C)C)C1] | 4-[(8aS,10aR)-5,6,7,8,8a, 10a- hexahydro-9,10-dioxa-1-aza- 3-anthrylamino]-2-{3- methoxy-4-[(1s,3S)-3- (dimethylamino)cyclobutoxy] phenylamino}pyrimidine |
| 191 | 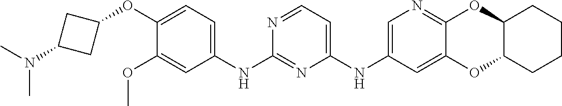 [SMILES: COc1cc(Nc2nccc(Nc3cnc4c(c3)O[C@H]3CCCC[C@@H]3O4)n2)ccc1O[C@H]1C[C@@H](N(C)C)C1] | 4-[(8aS,10aS)-5,6,7,8,8a,10a- hexahydro-9,10-dioxa-1-aza- 3-anthrylamino]-2-{3- methoxy-4-[(1s,3R)-3- (dimethylamino)cyclobutoxy] phenylamino}pyrimidine |
| 192 | 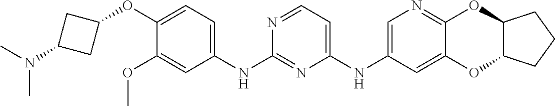 [SMILES: COc1cc(Nc2nccc(Nc3cnc4c(c3)O[C@H]3CCC[C@@H]3O4)n2)ccc1O[C@H]1C[C@@H](N(C)C)C1] | 4-{(3S,7S)-2,8-dioxa-10- azatricyclo[7.4.0.03,7]trideca- 1(13),9,11-trien-12-ylamino}- 2-{3-methoxy-4-[(1s,3R)-3- (dimethylamino)cyclobutoxy] phenylamino}pyrimidine |
| 193 | 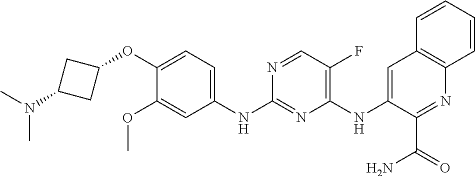 [SMILES: COc1cc(Nc2ncc(F)c(Nc3cc4ccccc4nc3C(N)=O)n2)ccc1O[C@H]1C[C@@H](N(C)C)C1] | 3-(5-fluoro-2-{3-methoxy-4- [(1s,3s)-3- (dimethylamino)cyclobutoxy] phenylamino}-4- pyrimidinylamino)-2- quinolinecarboxamide |
| 195 | 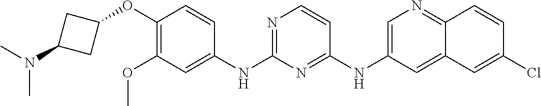 [SMILES: COc1cc(Nc2nccc(Nc3cnc4ccc(Cl)cc4c3)n2)ccc1O[C@H]1C[C@H](N(C)C)C1] | 4-(6-chloro-3-quinolylamino)- 2-{3-methoxy-4-[(1r,3r)-3- (dimethylamino)cyclobutoxy] phenylamino}pyrimidine |
| 198 | 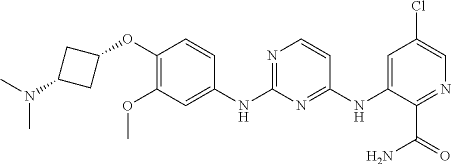 [SMILES: COc1cc(Nc2nccc(Nc3cc(Cl)cnc3C(N)=O)n2)ccc1O[C@H]1C[C@@H](N(C)C)C1] | 5-chloro-3-(2-{3-methoxy-4- [(1s,3s)-3- (dimethylamino)cyclobutoxy] phenylamino}-4- pyrimidinylamino)-2- pyridinecarboxamide |
| 199 | 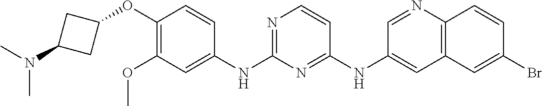 [SMILES: COc1cc(Nc2nccc(Nc3cnc4ccc(Br)cc4c3)n2)ccc1O[C@H]1C[C@H](N(C)C)C1] | 4-(6-bromo-3-quinolylamino)- 2-{3-methoxy-4-[(1r,3r)-3- (dimethylamino)cyclobutoxy] phenylamino}pyrimidine |
| 200 | 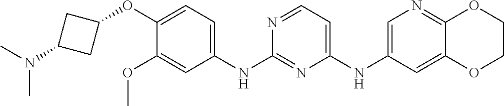 [SMILES: COc1cc(Nc2nccc(Nc3cnc4c(c3)OCCO4)n2)ccc1O[C@H]1C[C@@H](N(C)C)C1] | 4-(2,3-dihydro-1,4-dioxa-5- aza-7-naphthylamino)-2-{3- methoxy-4-[(1s,3s)-3- (dimethylamino)cyclobutoxy] phenylamino}pyrimidine |
| 202 | 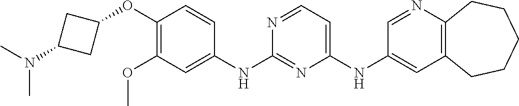 [SMILES: COc1cc(Nc2nccc(Nc3cnc4c(c3)CCCCC4)n2)ccc1O[C@H]1C[C@@H](N(C)C)C1] | 4-{8-azabicyclo[5.4.0]undeca- 1(7),8,10-trien-10-ylamino}- 2-{3-methoxy-4-[(1s,3s)-3- (dimethylamino)cyclobutoxy] phenylamino}pyrimidine |
| 203 | 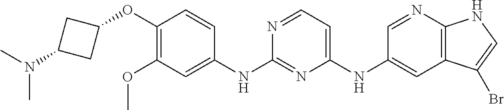 [SMILES: COc1cc(Nc2nccc(Nc3cnc4[nH]cc(Br)c4c3)n2)ccc1O[C@H]1C[C@@H](N(C)C)C1] | 4-(3-bromo-1H-1,7- diazainden-5-ylamino)-2-{3- methoxy-4-[(1s,3s)-3- (dimethylamino)cyclobutoxy] phenylamino}pyrimidine |
| 204 | 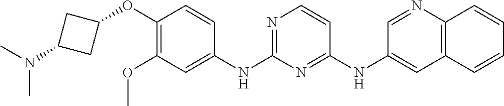 [SMILES: COc1cc(Nc2nccc(Nc3cnc4ccccc4c3)n2)ccc1O[C@H]1C[C@@H](N(C)C)C1] | 2-{3-methoxy-4-[(1s,3s)-3- (dimethylamino)cyclobutoxy] phenylamino}-4-(3- quinolylamino)pyrimidine |
| 210 | 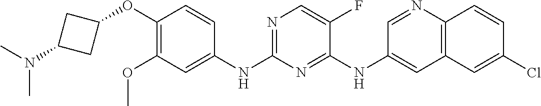 [SMILES: COc1cc(Nc2ncc(F)c(Nc3cnc4ccc(Cl)cc4c3)n2)ccc1O[C@H]1C[C@@H](N(C)C)C1] | 4-(6-chloro-3-quinolylamino)- 5-fluoro-2-{3-methoxy-4- [(1s,3s)-3- (dimethylamino)cyclobutoxy] phenylamino}pyrimidine |
| 252 | 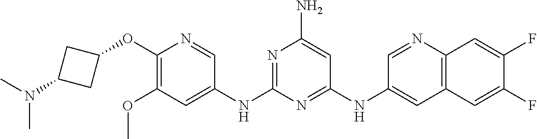 [SMILES: COc1cc(Nc2nc(N)cc(Nc3cnc4cc(F)c(F)cc4c3)n2)cnc1O[C@H]1C[C@@H](N(C)C)C1] | 6-amino-4-(6,7-difluoro-3- quinolylamino)-2-{5- methoxy-6-[(1s,3s)-3- (dimethylamino)cyclobutoxy]- 3-pyridylamino}pyrimidine |
| 253 | 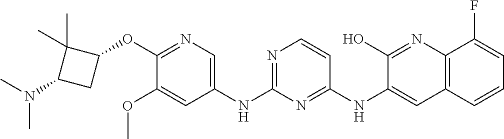 [SMILES: COc1cc(Nc2nccc(Nc3cc4cccc(F)c4nc3O)n2)cnc1O[C@@H]1C[C@H](N(C)C)C1(C)C] | 3-(2-{6-[(1R,3S)-3- (dimethylamino)-2,2- dimethylcyclobutoxy]-5- methoxy-3-pyridylamino}-4- pyrimidinylamino)-8-fluoro- 2-quinolinol |
| 254 | 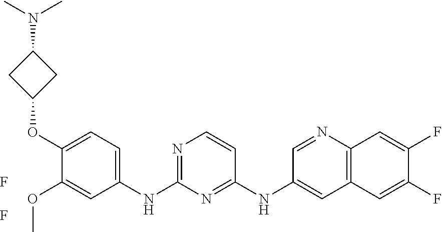 | 2-{6-[(1R,3S)-3- (dimethylamino)-2,2- dimethylcyclobutoxy]-5- methoxy-3-pyridylamino}-4- (6,7-difluoro-3- quinolylamino)pyrimidine |
| 255 | 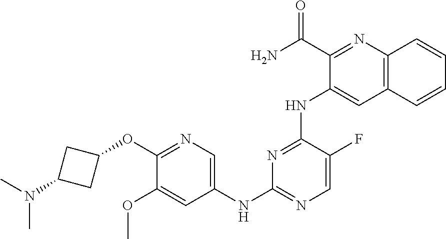 [SMILES: COc1cc(Nc2ncc(F)c(Nc3cc4ccccc4nc3C(N)=O)n2)cnc1O[C@H]1C[C@@H](N(C)C)C1] | 3-(5-fluoro-2-{5-methoxy-6- [(1s,3s)-3- (dimethylamino)cyclobutoxy]- 3-pyridylamino}-4- pyrimidinylamino)-2- quinolinecarboxamide |
| 256 | 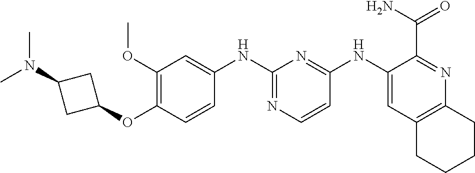 [SMILES: COc1cc(Nc2nccc(Nc3cc4c(nc3C(N)=O)CCCC4)n2)ccc1O[C@H]1C[C@@H](N(C)C)C1] | 3-(2-{3-methoxy-4-[(1s,3s)-3- (dimethylamino)cyclobutoxy] phenylamino}-4- pyrimidinylamino)-5,6,7,8- tetrahydro-2- quinolinecarboxamide |
| 257 | 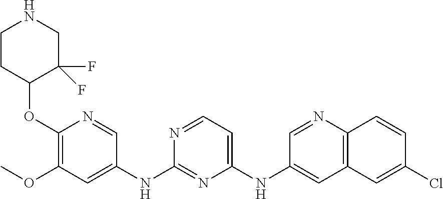 [SMILES: COc1cc(Nc2nccc(Nc3cnc4ccc(Cl)cc4c3)n2)cnc1OC1CCNCC1(F)F] | 4-(6-chloro-3-quinolylamino)- 2-[6-(3,3-difluoro-4- piperidyloxy)-5-methoxy-3- pyridylamino]pyrimidine |
| 258 | 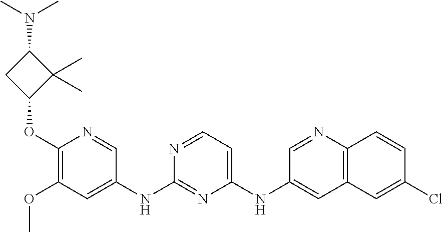 [SMILES: COc1cc(Nc2nccc(Nc3cnc4ccc(Cl)cc4c3)n2)cnc1O[C@@H]1C[C@H](N(C)C)C1(C)C] | 2-{6-[(1R,3S)-3- (dimethylamino)-2,2- dimethylcyclobutoxy]-5- methoxy-3-pyridylamino}-4- (6-chloro-3- quinolylamino)pyrimidine |
| 259 | 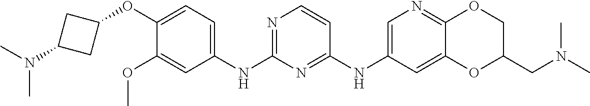 | 4-{2- [(dimethylamino)methyl]-2,3- dihydro-1,4-dioxa-5-aza-7- naphthylamino}-2-{3- methoxy-4-[(1s,3s)-3- (dimethylamino)cyclobutoxy] phenylamino}pyrimidine |
| 260 | 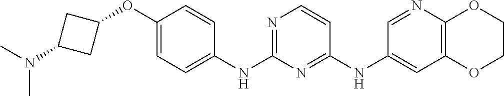 [SMILES: CN(C)[C@H]1C[C@@H](Oc2ccc(Nc3nccc(Nc4cnc5c(c4)OCCO5)n3)cc2)C1] | 4-(2,3-dihydro-1,4-dioxa-5- aza-7-naphthylamino)-2-{p- [(1s,3s)-3- (dimethylamino)cyclobutoxy] phenylamino}pyrimidine |
| 261 | 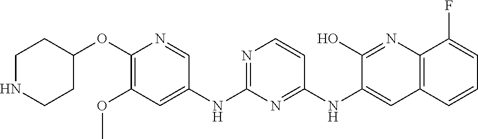 [SMILES: COc1cc(Nc2nccc(Nc3cc4cccc(F)c4nc3O)n2)cnc1OC1CCNCC1] | 8-fluoro-3-{2-[5-methoxy-6- (4-piperidyloxy)-3- pyridylamino]-4- pyrimidinylamino}-2- quinolinol |
| 262 | 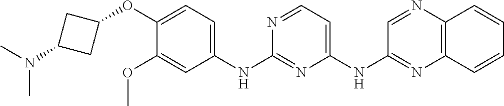 [SMILES: COc1cc(Nc2nccc(Nc3cnc4ccccc4n3)n2)ccc1O[C@H]1C[C@@H](N(C)C)C1] | 2-{3-methoxy-4-[(1s,3s)-3- (dimethylamino)cyclobutoxy] phenylamino}-4-(2- quinoxalinylamino)pyridine |
| 263 | 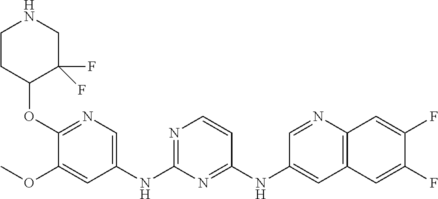 [SMILES: COc1cc(Nc2nccc(Nc3cnc4cc(F)c(F)cc4c3)n2)cnc1OC1CCNCC1(F)F] | 2-[6-(3,3-difluoro-4- piperidyloxy)-5-methoxy-3- pyridylamino]-4-(6,7- difluoro-3- quinolylamino)pyrimidine |
| 264 | 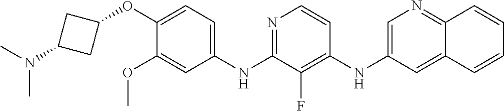 [SMILES: COc1cc(Nc2nccc(Nc3cnc4ccccc4c3)c2F)ccc1O[C@H]1C[C@@H](N(C)C)C1] | 3-fluoro-2-{3-methoxy-4- [(1s,3s)-3- (dimethylamino)cyclobutoxy] phenylamino}-4-(3- quinolylamino)pyridine |
| 265 | 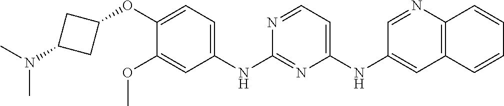 [SMILES: COc1cc(Nc2nccc(Nc3cnc4ccccc4c3)n2)ccc1O[C@H]1C[C@@H](N(C)C)C1] | 2-{3-methoxy-4-[(1s,3s)-3- (dimethylamino)cyclobutoxy] phenylamino}-4-(3- quinolylamino)pyridine |
| 266 | 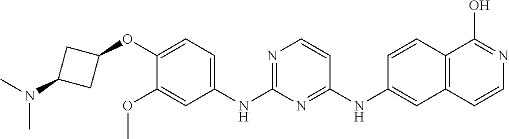 [SMILES: COc1cc(Nc2nccc(Nc3ccc4c(O)nccc4c3)n2)ccc1O[C@H]1C[C@@H](N(C)C)C1] | 6-(2-{3-methoxy-4-[(1s,3s)-3- (dimethylamino)cyclobutoxy] phenylamino}-4- pyrimidinylamino)-1- isoquinolinol |
| 267 | 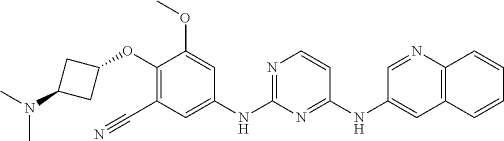 [SMILES: COc1cc(Nc2nccc(Nc3cnc4ccccc4c3)n2)cc(C#N)c1O[C@H]1C[C@H](N(C)C)C1] | 3-methoxy-5-[4-(3- quinolylamino)-2- pyrimidinylamino]-2-[(1r,3r)- 3- (dimethylamino)cyclobutoxy] benzonitrile |
| 268 | 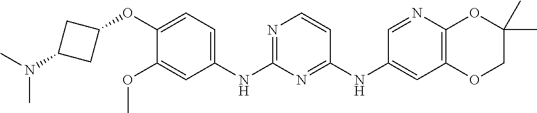 | 4-(3,3-dimethyl-2,3-dihydro- 1,4-dioxa-5-aza-7- naphthylamino)-2-{3- methoxy-4-[(1s,3s)-3- (dimethylamino)cyclobutoxy] phenylamino}pyrimidine |
| 269 | 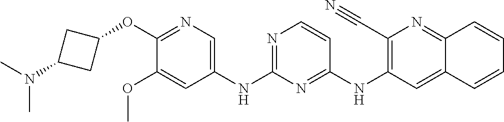 [SMILES: COc1cc(Nc2nccc(Nc3cc4ccccc4nc3C#N)n2)cnc1O[C@H]1C[C@@H](N(C)C)C1] | 3-(2-{5-methoxy-6-[(1s,3s)-3- (dimethylamino)cyclobutoxy]- 3-pyridylamino}-4- pyrimidinylamino)-2- quinolinecarbonitrile |
| 270 | 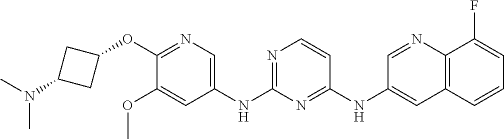 [SMILES: COc1cc(Nc2nccc(Nc3cnc4c(F)cccc4c3)n2)cnc1O[C@H]1C[C@@H](N(C)C)C1] | 4-(8-fluoro-3-quinolylamino)- 2-{5-methoxy-6-[(1s,3s)-3- (dimethylamino)cyclobutoxy]- 3-pyridylamino}pyrimidine |
| 271 | 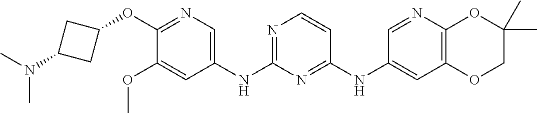 | 4-(3,3-dimethyl-2,3-dihydro- 1,4-dioxa-5-aza-7- naphthylamino)-2-{5- methoxy-6-[(1s,3s)-3- (dimethylamino)cyclobutoxy]- 3-pyridylamino}pyrimidine |
| 272 | 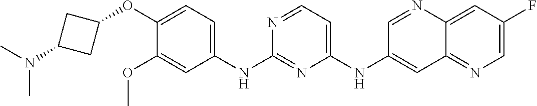 [SMILES: COc1cc(Nc2nccc(Nc3cnc4cc(F)cnc4c3)n2)ccc1O[C@H]1C[C@@H](N(C)C)C1] | 4-(7-fluoro-1,5-diaza-3- naphthylamino)-2-{3- methoxy-4-[(1s,3s)-3- (dimethylamino)cyclobutoxy] phenylamino}pyrimidine |
| 273 | 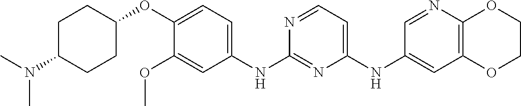 [SMILES: COc1cc(Nc2nccc(Nc3cnc4c(c3)OCCO4)n2)ccc1O[C@H]1CC[C@@H](N(C)C)CC1] | 4-(2,3-dihydro-1,4-dioxa-5- aza-7-naphthylamino)-2-{3- methoxy-4-[(1s,4s)-4- (dimethylamino) cyclohexyloxylphenylamino} pyrimidine |
| 274 | 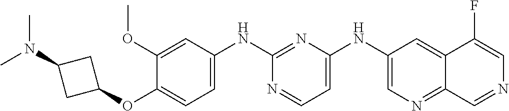 [SMILES: COc1cc(Nc2nccc(Nc3cnc4cncc(F)c4c3)n2)ccc1O[C@H]1C[C@@H](N(C)C)C1] | 4-(5-fluoro-1,7-diaza-3- naphthylamino)-2-{3- methoxy-4-[(1s,3s)-3- (dimethylamino)cyclobutoxy] phenylamino}pyrimidine |
| 275 | 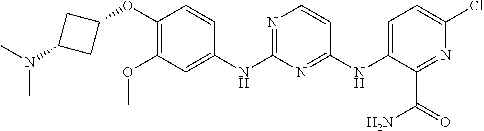 [SMILES: COc1cc(Nc2nccc(Nc3ccc(Cl)nc3C(N)=O)n2)ccc1O[C@H]1C[C@@H](N(C)C)C1] | 6-chloro-3-(2-{3-methoxy-4- [(1s,3s)-3- (dimethylamino)cyclobutoxy] phenylamino}-4- pyrimidinylamino)-2- pyridinecarboxamide |
| 276 | 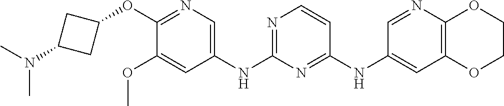 [SMILES: COc1cc(Nc2nccc(Nc3cnc4c(c3)OCCO4)n2)cnc1O[C@H]1C[C@@H](N(C)C)C1] | 4-(2,3-dihydro-1,4-dioxa-5- aza-7-naphthylamino)-2-{5- methoxy-6-[(1s,3s)-3- (dimethylamino)cyclobutoxy]- 3-pyridylamino}pyrimidine |
| 277 | 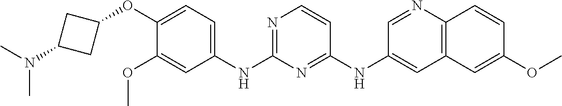 | 4-(6-methoxy-3- quinolylamino)-2-{3- methoxy-4-[(1s,3s)-3- (dimethylamino)cyclobutoxy] phenylamino}pyrimidine |
| 278 | 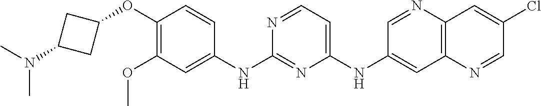 [SMILES: COc1cc(Nc2nccc(Nc3cnc4cc(Cl)cnc4c3)n2)ccc1O[C@H]1C[C@@H](N(C)C)C1] | 4-(7-chloro-1,5-diaza-3- naphthylamino)-2-{3- methoxy-4-[(1s,3s)-3- (dimethylamino)cyclobutoxy] phenylamino}pyrimidine |
| 279 | 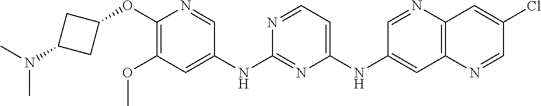 [SMILES: COc1cc(Nc2nccc(Nc3cnc4cc(Cl)cnc4c3)n2)cnc1O[C@H]1C[C@@H](N(C)C)C1] | 4-(7-chloro-1,5-diaza-3- naphthylamino)-2-{5- methoxy-6-[(1s,3s)-3- (dimethylamino)cyclobutoxy]- 3-pyridylamino}pyrimidine |
| 280 | 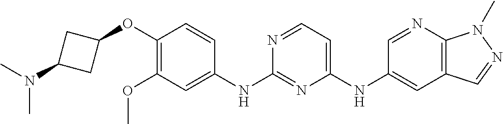 [SMILES: COc1cc(Nc2nccc(Nc3cnc4c(cnn4C)c3)n2)ccc1O[C@H]1C[C@@H](N(C)C)C1] | 2-{3-methoxy-4-[(1s,3s)-3- (dimethylamino)cyclobutoxy] phenylamino}-4-(1-methyl- 1H-1,2,7-triazainden-5- ylamino)pyrimidine |
| 281 | 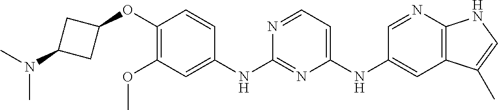 [SMILES: COc1cc(Nc2nccc(Nc3cnc4[nH]cc(C)c4c3)n2)ccc1O[C@H]1C[C@@H](N(C)C)C1] | 2-{3-methoxy-4-[(1s,3s)-3- (dimethylamino)cyclobutoxy] phenylamino}-4-(3-methyl- 1H-1,7-diazainden-5- ylamino)pyrimidine |
| 282 | 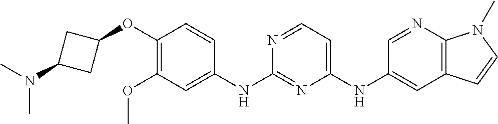 [SMILES: COc1cc(Nc2nccc(Nc3cnc4c(ccn4C)c3)n2)ccc1O[C@H]1C[C@@H](N(C)C)C1] | 2-{3-methoxy-4-[(1s,3s)-3- (dimethylamino)cyclobutoxy] phenylamino}-4-(1-methyl- 1H-1,7-diazainden-5- ylamino)pyrimidine |
| 283 | 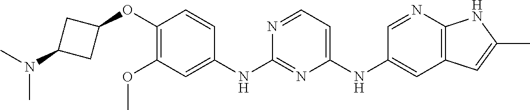 | 2-{3-methoxy-4-[(1s,3s)-3- (dimethylamino)cyclobutoxy] phenylamino}-4-(2-methyl- 1H-1,7-diazainden-5- ylamino)pyrimidine |
| 284 | 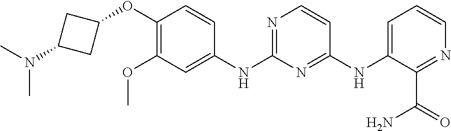 [SMILES: COc1cc(Nc2nccc(Nc3cccnc3C(N)=O)n2)ccc1O[C@H]1C[C@@H](N(C)C)C1] | 3-(2-{3-methoxy-4-[(1s,3s)-3- (dimethylamino)cyclobutoxy] phenylamino}-4- pyrimidinylamino)-2- pyridinecarboxamide |
| 285 | 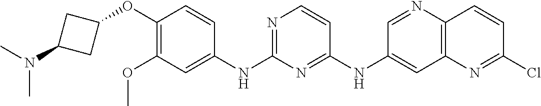 [SMILES: COc1cc(Nc2nccc(Nc3cnc4ccc(Cl)nc4c3)n2)ccc1O[C@H]1C[C@H](N(C)C)C1] | 4-(6-chloro-1,5-diaza-3- naphthylamino)-2-{3- methoxy-4-[(1r,3r)-3- (dimethylamino)cyclobutoxy] phenylamino}pyrimidine |
| 286 | 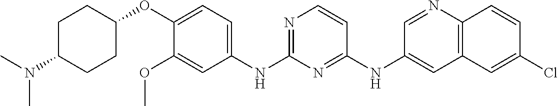 [SMILES: COc1cc(Nc2nccc(Nc3cnc4ccc(Cl)cc4c3)n2)ccc1O[C@H]1CC[C@@H](N(C)C)CC1] | 4-(6-chloro-3-quinolylamino)- 2-{3-methoxy-4-[(1s,4s)-4- (dimethylamino)cyclohexyloxy] phenylamino}pyrimidine |
| 287 | 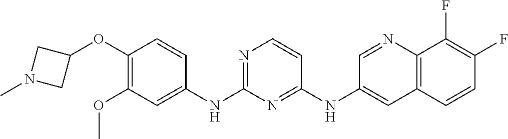 [SMILES: COc1cc(Nc2nccc(Nc3cnc4c(F)c(F)ccc4c3)n2)ccc1OC1CN(C)C1] | 4-(7,8-difluoro-3- quinolylamino)-2-[3- methoxy-4-(1-methyl-3- azetidinyloxy)phenylamino] pyrimidine |
| 288 | 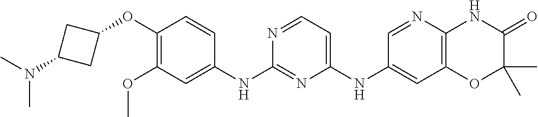 | 7-(2-{3-methoxy-4-[(1s,3s)-3- (dimethylamino)cyclobutoxy] phenylamino}-4- pyrimidinylamino)-2,2- dimethyl-2,4-dihydro-1-oxa- 4,5-diaza-3-naphthalenone |
| 289 | 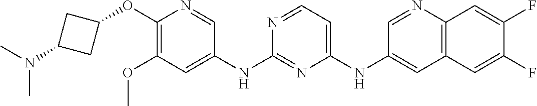 [SMILES: COc1cc(Nc2nccc(Nc3cnc4cc(F)c(F)cc4c3)n2)cnc1O[C@H]1C[C@@H](N(C)C)C1] | 4-(6,7-difluoro-3- quinolylamino)-2-{5- methoxy-6-[(1s,3s)-3- (dimethylamino)cyclobutoxy]- 3-pyridylamino}pyrimidine |
| 290 | 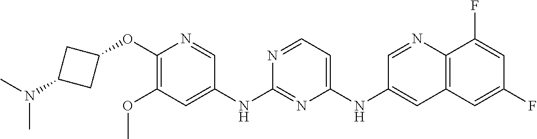 [SMILES: COc1cc(Nc2nccc(Nc3cnc4c(F)cc(F)cc4c3)n2)cnc1O[C@H]1C[C@@H](N(C)C)C1] | 4-(6,8-difluoro-3- quinolylamino)-2-{5- methoxy-6-[(1s,3s)-3- (dimethylamino)cyclobutoxy]- 3-pyridylamino}pyrimidine |
| 291 | 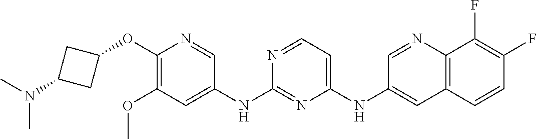 [SMILES: COc1cc(Nc2nccc(Nc3cnc4c(F)c(F)ccc4c3)n2)cnc1O[C@H]1C[C@@H](N(C)C)C1] | 4-(7,8-difluoro-3- quinolylamino)-2-{5- methoxy-6-[(1s,3s)-3- (dimethylamino)cyclobutoxy]- 3-pyridylamino}pyrimidine |
| 292 | 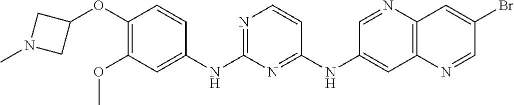 [SMILES: COc1cc(Nc2nccc(Nc3cnc4cc(Br)cnc4c3)n2)ccc1OC1CN(C)C1] | 4-(7-bromo-1,5-diaza-3- naphthylamino)-2-[3- methoxy-4-(1-methyl-3- azetidinyloxy)phenylamino] pyrimidine |
| 293 | 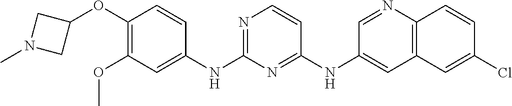 [SMILES: COc1cc(Nc2nccc(Nc3cnc4ccc(Cl)cc4c3)n2)ccc1OC1CN(C)C1] | 4-(6-chloro-3-quinolylamino)- 2-[3-methoxy-4-(1-methyl-3- azetidinyloxy)phenylamino] pyrimidine |
| 294 | 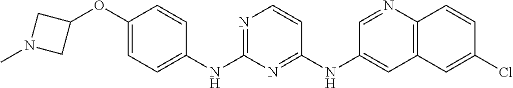 [SMILES: CN1CC(Oc2ccc(Nc3nccc(Nc4cnc5ccc(Cl)cc5c4)n3)cc2)C1] | 4-(6-chloro-3-quinolylamino)- 2-[p-(1-methyl-3- azetidinyloxy)phenylamino] pyrimidine |
| 295 | 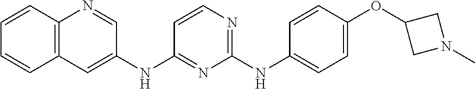 [SMILES: CN1CC(Oc2ccc(Nc3nccc(Nc4cnc5ccccc5c4)n3)cc2)C1] | 2-[p-(1-methyl-3- azetidinyloxy)phenylamino]- 4-(3- quinolylamino)pyrimidine |
| 296 | 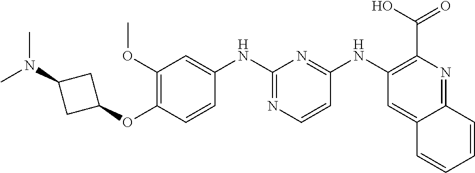 [SMILES: COc1cc(Nc2nccc(Nc3cc4ccccc4nc3C(=O)O)n2)ccc1O[C@H]1C[C@@H](N(C)C)C1] | 3-(2-{3-methoxy-4-[(1s,3s)-3- (dimethylamino)cyclobutoxy] phenylamino}-4- pyrimidinylamino)-2- quinolinecarboxylic acid |
| 297 | 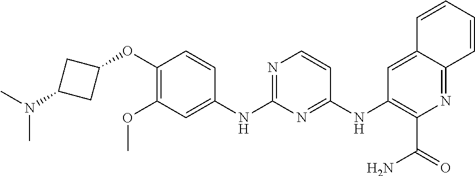 [SMILES: COc1cc(Nc2nccc(Nc3cc4ccccc4nc3C(N)=O)n2)ccc1O[C@H]1C[C@@H](N(C)C)C1] | 3-(2-{3-methoxy-4-[(1s,3s)-3- (dimethylamino)cyclobutoxy] phenylamino}-4- pyrimidinylamino)-2- quinolinecarboxamide |
| 298 | 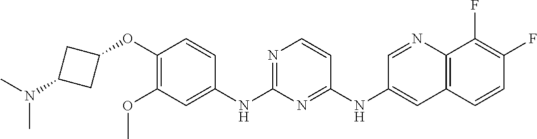 [SMILES: COc1cc(Nc2nccc(Nc3cnc4c(F)c(F)ccc4c3)n2)ccc1O[C@H]1C[C@@H](N(C)C)C1] | 4-(7,8-difluoro-3- quinolylamino)-2-{3- methoxy-4-[(1s,3s)-3- (dimethylamino)cyclobutoxy] phenylamino}pyrimidine |
| 299 | 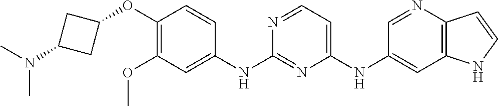 [SMILES: COc1cc(Nc2nccc(Nc3cnc4cc[nH]c4c3)n2)ccc1O[C@H]1C[C@@H](N(C)C)C1] | 4-(1H-1,4-diazainden-6- ylamino)-2-{3-methoxy-4- [(1s,3s)-3- (dimethylamino)cyclobutoxy] phenylamino}pyrimidine |
| 300 | 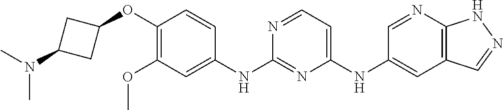 [SMILES: COc1cc(Nc2nccc(Nc3cnc4[nH]ncc4c3)n2)ccc1O[C@H]1C[C@@H](N(C)C)C1] | 2-{3-methoxy-4-[(1s,3s)-3- (dimethylamino)cyclobutoxy] phenylamino}-4-(1H-1,2,7- triazainden-5- ylamino)pyrimidine |
| 301 | 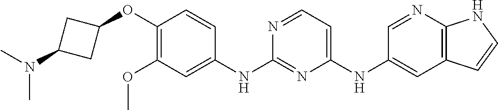 [SMILES: COc1cc(Nc2nccc(Nc3cnc4[nH]ccc4c3)n2)ccc1O[C@H]1C[C@@H](N(C)C)C1] | 4-(1H-1,7-diazainden-5- ylamino)-2-{3-methoxy-4- [(1s,3s)-3- (dimethylamino)cyclobutoxy] phenylamino}pyrimidine |
| 302 | 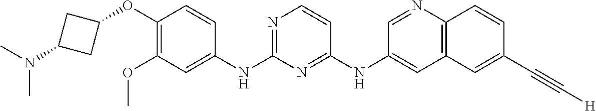 [SMILES: C#Cc1ccc2ncc(Nc3ccnc(Nc4ccc(O[C@H]5C[C@@H](N(C)C)C5)c(OC)c4)n3)cc2c1] | 4-(6-ethynyl-3- quinolylamino)-2-{3- methoxy-4-[(1s,3s)-3- (dimethylamino)cyclobutoxy] phenylamino}pyrimidine |
| 304 | 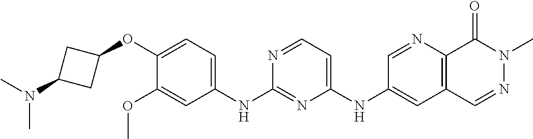 | 3-(2-{3-methoxy-4-[(1s,3s)-3- (dimethylamino)cyclobutoxy] phenylamino}-4- pyrimidinylamino)-7-methyl- 1,6,7-triaza-8(7H)- naphthalenone |
| 305 | 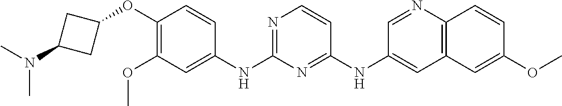 | 4-(6-methoxy-3- quinolylamino)-2-{3- methoxy-4-[(1r,3r)-3- (dimethylamino)cyclobutoxy] phenylamino}pyrimidine |
| 306 | 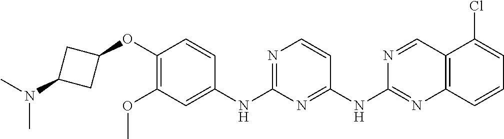 [SMILES: COc1cc(Nc2nccc(Nc3ncc4c(Cl)cccc4n3)n2)ccc1O[C@H]1C[C@@H](N(C)C)C1] | 4-(5-chloro-2- quinazolinylamino)-2-{3- methoxy-4-[(1s,3s)-3- (dimethylamino)cyclobutoxy] phenylamino}pyrimidine |
| 307 | 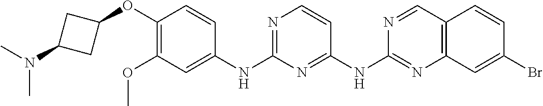 [SMILES: COc1cc(Nc2nccc(Nc3ncc4ccc(Br)cc4n3)n2)ccc1O[C@H]1C[C@@H](N(C)C)C1] | 4-(7-bromo-2- quinazolinylamino)-2-{3- methoxy-4-[(1s,3s)-3- (dimethylamino)cyclobutoxy] phenylamino}pyrimidine |
| 308 | 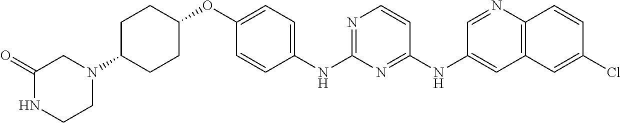 [SMILES: O=C1CN([C@H]2CC[C@@H](Oc3ccc(Nc4nccc(Nc5cnc6ccc(Cl)cc6c5)n4)cc3)CC2)CCN1] | 4-[(1s,4s)-4-{p-[4-(6-chloro- 3-quinolylamino)-2- pyrimidinylaminophenoxy} cyclohexyl]-2-piperazinone |
| 309 | 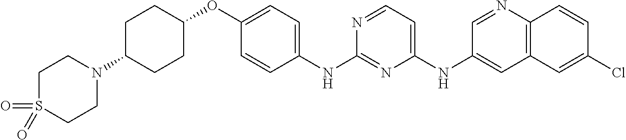 [SMILES: O=S1(=O)CCN([C@H]2CC[C@@H](Oc3ccc(Nc4nccc(Nc5cnc6ccc(Cl)cc6c5)n4)cc3)CC2)CC1] | 4-[(1s,4s)-4-{p-[4-(6-chloro- 3-quinolylamino)-2- pyrimidinylaminophenoxy} cyclohexyl]-126,4-thiazinane- 1,1-dione |
| 310 | 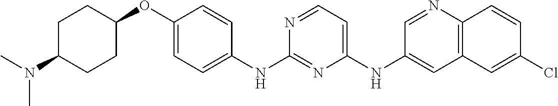 [SMILES: CN(C)[C@H]1CC[C@@H](Oc2ccc(Nc3nccc(Nc4cnc5ccc(Cl)cc5c4)n3)cc2)CC1] | 4-(6-chloro-3-quinolylamino)- 2-{p-[(1s,4s)-4- (dimethylamino)cyclohexyloxy] phenylamino}pyrimidine |
| 311 | 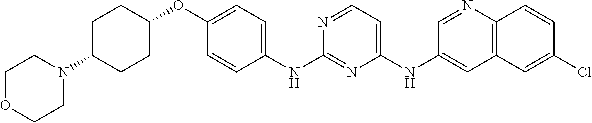 [SMILES: Clc1ccc2ncc(Nc3ccnc(Nc4ccc(O[C@H]5CC[C@@H](N6CCOCC6)CC5)cc4)n3)cc2c1] | 4-(6-chloro-3-quinolylamino)- 2-{p-[(1s,4s)-4- morpholinocyclohexyloxy] phenylamino}pyrimidine |
| 312 | 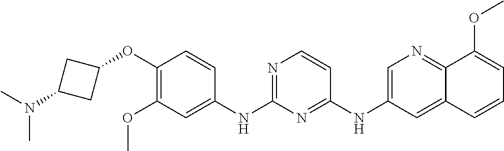 | 4-(8-methoxy-3- quinolylamino)-2-{3- methoxy-4-[(1s,3s)-3- (dimethylamino)cyclobutoxy] phenylamino}pyrimidine |
| 313 | 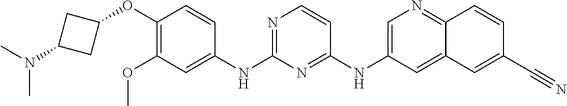 [SMILES: COc1cc(Nc2nccc(Nc3cnc4ccc(C#N)cc4c3)n2)ccc1O[C@H]1C[C@@H](N(C)C)C1] | 3-(2-{3-methoxy-4-[(1s,3s)-3- (dimethylamino)cyclobutoxy] phenylamino}-4- pyrimidinylamino)-6- quinolinecarbonitrile |
| 314 | 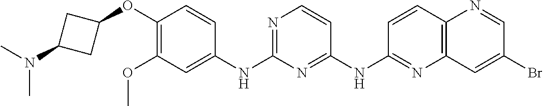 [SMILES: COc1cc(Nc2nccc(Nc3ccc4ncc(Br)cc4n3)n2)ccc1O[C@H]1C[C@@H](N(C)C)C1] | 4-(7-bromo-1,5-diaza-2- naphthylamino)-2-{3- methoxy-4-[(1s,3s)-3- (dimethylamino)cyclobutoxy] phenylamino}pyrimidine |
| 315 | 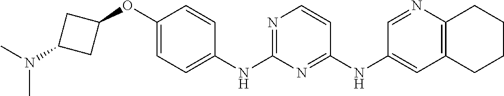 [SMILES: CN(C)[C@H]1C[C@H](Oc2ccc(Nc3nccc(Nc4cnc5c(c4)CCCC5)n3)cc2)C1] | 2-{p-[(1r,3r)-3- (dimethylamino)cyclobutoxy] phenylamino}-4-(5,6,7,8- tetrahydro-3- quinolylamino)pyrimidine |
| 316 | 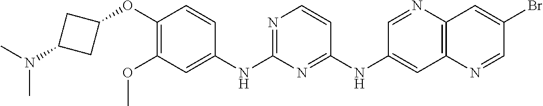 [SMILES: COc1cc(Nc2nccc(Nc3cnc4cc(Br)cnc4c3)n2)ccc1O[C@H]1C[C@@H](N(C)C)C1] | 4-(7-bromo-1,5-diaza-3- naphthylamino)-2-{3- methoxy-4-[(1s,3s)-3- (dimethylamino)cyclobutoxy] phenylamino}pyrimidine |
| 317 | 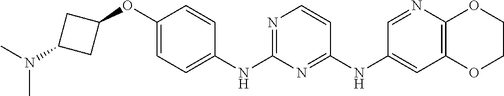 [SMILES: CN(C)[C@H]1C[C@H](Oc2ccc(Nc3nccc(Nc4cnc5c(c4)OCCO5)n3)cc2)C1] | 4-(2,3-dihydro-1,4-dioxa-5- aza-7-naphthylamino)-2-{p- [(1r,3r)-3- (dimethylamino)cyclobutoxy] phenylamino}pyrimidine |
| 318 | 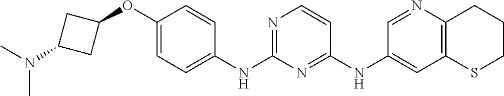 [SMILES: CN(C)[C@H]1C[C@H](Oc2ccc(Nc3nccc(Nc4cnc5c(c4)SCCC5)n3)cc2)C1] | 4-(3,4-dihydro-2H-1-thia-5- azanaphth-7-ylamino)-2-{p- [(1r,3r)-3- (dimethylamino)cyclobutoxy] phenylamino}pyrimidine |
| 319 | 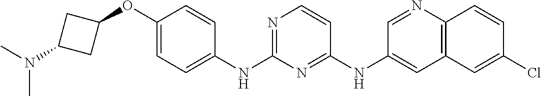 [SMILES: CN(C)[C@H]1C[C@H](Oc2ccc(Nc3nccc(Nc4cnc5ccc(Cl)cc5c4)n3)cc2)C1] | 4-(6-chloro-3-quinolylamino)- 2-{p-[(1r,3r)-3- (dimethylamino)cyclobutoxy] phenylamino}pyrimidine |
| 320 | 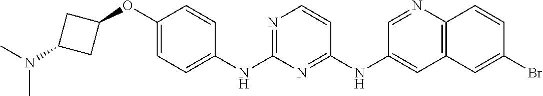 [SMILES: CN(C)[C@H]1C[C@H](Oc2ccc(Nc3nccc(Nc4cnc5ccc(Br)cc5c4)n3)cc2)C1] | 4-(6-bromo-3-quinolylamino)- 2-{p-[(1r,3r)-3- (dimethylamino)cyclobutoxy] phenylamino}pyrimidine |
| 321 | 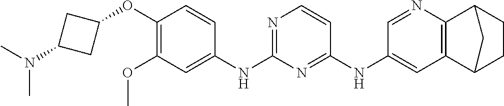 [SMILES: COc1cc(Nc2nccc(Nc3cnc4c(c3)C3CCC4C3)n2)ccc1O[C@H]1C[C@@H](N(C)C)C1] | 4-{3- azatricyclo[6.2.1.02,7]undeca- 2(7),3,5-trien-5-ylamino}-2- {3-methoxy-4-[(1s,3s)-3- (dimethylamino)cyclobutoxy] phenylamino}pyrimidine |
| 322 | 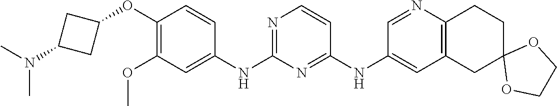 [SMILES: COc1cc(Nc2nccc(Nc3cnc4c(c3)CC3(CC4)OCCO3)n2)ccc1O[C@H]1C[C@@H](N(C)C)C1] | 4-(7′,8′-dihydro-5′H-spiro[1,3- dioxolane-2,6′-quinolin]-3′- ylamino)-2-{3-methoxy-4- [(1s,3s)-3- (dimethylamino)cyclobutoxy] phenylamino}pyrimidine |
| 323 | 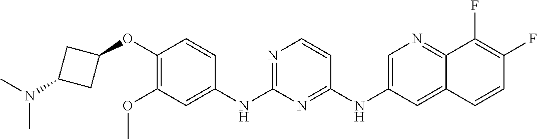 [SMILES: COc1cc(Nc2nccc(Nc3cnc4c(F)c(F)ccc4c3)n2)ccc1O[C@H]1C[C@H](N(C)C)C1] | 4-(7,8-difluoro-3- quinolylamino)-2-{3- methoxy-4-[(1r,3r)-3- (dimethylamino)cyclobutoxy] phenylamino}pyrimidine |
| 324 | 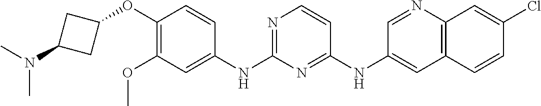 [SMILES: COc1cc(Nc2nccc(Nc3cnc4cc(Cl)ccc4c3)n2)ccc1O[C@H]1C[C@H](N(C)C)C1] | 4-(7-chloro-3-quinolylamino)- 2-{3-methoxy-4-[(1r,3r)-3- (dimethylamino)cyclobutoxy] phenylamino}pyrimidine |
| 325 | 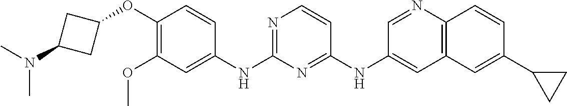 [SMILES: COc1cc(Nc2nccc(Nc3cnc4ccc(C5CC5)cc4c3)n2)ccc1O[C@H]1C[C@H](N(C)C)C1] | 4-(6-cyclopropyl-3- quinolylamino)-2-{3- methoxy-4-[(1r,3r)-3- (dimethylamino)cyclobutoxy] phenylamino}pyrimidine |
| 326 | 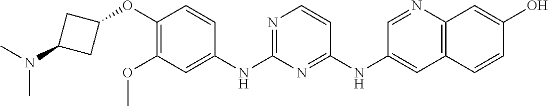 [SMILES: COc1cc(Nc2nccc(Nc3cnc4cc(O)ccc4c3)n2)ccc1O[C@H]1C[C@H](N(C)C)C1] | 3-(2-{3-methoxy-4-[(1r,3r)-3- (dimethylamino)cyclobutoxy] phenylamino}-4- pyrimidinylamino)-7- quinolinol |
| 327 | 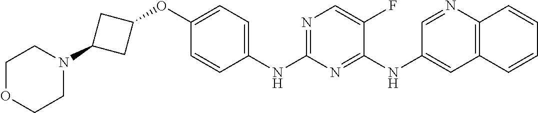 [SMILES: Fc1cnc(Nc2ccc(O[C@H]3C[C@H](N4CCOCC4)C3)cc2)nc1Nc1cnc2ccccc2c1] | 5-fluoro-4-(3-quinolylamino)- 2-{p-[(1r,3r)-3- morpholinocyclobutoxy] phenylamino}pyrimidine |
| 328 | 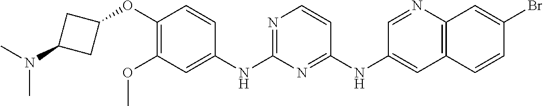 [SMILES: COc1cc(Nc2nccc(Nc3cnc4cc(Br)ccc4c3)n2)ccc1O[C@H]1C[C@H](N(C)C)C1] | 4-(7-bromo-3-quinolylamino)- 2-{3-methoxy-4-[(1r,3r)-3- (dimethylamino)cyclobutoxy] phenylamino}pyrimidine |
| 329 | 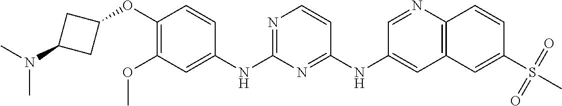 | 4-(6-mesyl-3-quinolylamino)- 2-{3-methoxy-4-[(1r,3r)-3- (dimethylamino)cyclobutoxy] phenylamino}pyrimidine |
| 330 | 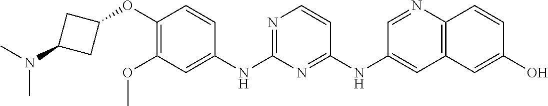 [SMILES: COc1cc(Nc2nccc(Nc3cnc4ccc(O)cc4c3)n2)ccc1O[C@H]1C[C@H](N(C)C)C1] | 3-(2-{3-methoxy-4-[(1r,3r)-3- (dimethylamino)cyclobutoxy] phenylamino}-4- pyrimidinylamino)-6- quinolinol |
| 331 | 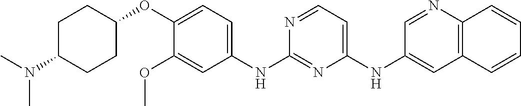 [SMILES: COc1cc(Nc2nccc(Nc3cnc4ccccc4c3)n2)ccc1O[C@H]1CC[C@@H](N(C)C)CC1] | 2-{3-methoxy-4-[(1s,4s)-4- (dimethylamino) cyclohexyloxylphenylamino}- 4-(3- quinolylamino)pyrimidine |
| 332 | 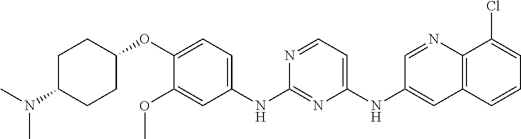 [SMILES: COc1cc(Nc2nccc(Nc3cnc4c(Cl)cccc4c3)n2)ccc1O[C@H]1CC[C@@H](N(C)C)CC1] | 4-(8-chloro-3-quinolylamino)- 2-{3-methoxy-4-[(1s,4s)-4- (dimethylamino)cyclohexyloxy] phenylamino}pyrimidine |
| 333 | 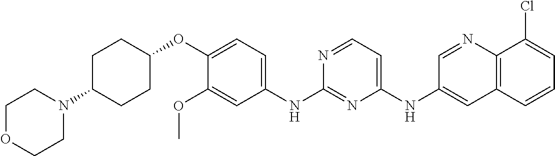 [SMILES: COc1cc(Nc2nccc(Nc3cnc4c(Cl)cccc4c3)n2)ccc1O[C@H]1CC[C@@H](N2CCOCC2)CC1] | 4-(8-chloro-3-quinolylamino)- 2-{3-methoxy-4-[(1s,4s)-4- morpholinocyclohexyloxy] phenylamino}pyrimidine |
| 334 | 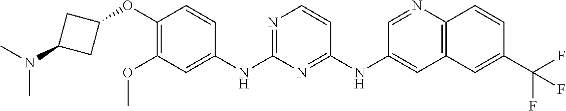 [SMILES: COc1cc(Nc2nccc(Nc3cnc4ccc(C(F)(F)F)cc4c3)n2)ccc1O[C@H]1C[C@H](N(C)C)C1] | 2-{3-methoxy-4-[(1r,3r)-3- (dimethylamino)cyclobutoxy] phenylamino}-4-[6- (trifluoromethyl)-3- quinolylamino]pyrimidine |
| 335 | 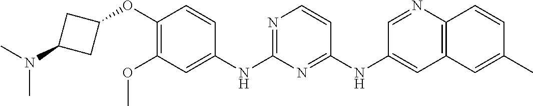 | 2-{3-methoxy-4-[(1r,3r)-3- (dimethylamino)cyclobutoxy] phenylamino}-4-(6-methyl-3- quinolylamino)pyrimidine |
| 336 | 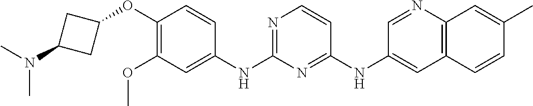 | 2-{3-methoxy-4-[(1r,3r)-3- (dimethylamino)cyclobutoxy] phenylamino}-4-(7-methyl-3- quinolylamino)pyrimidine |
| 337 | 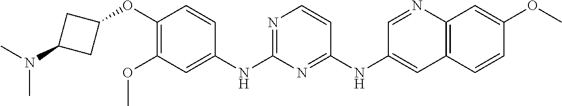 | 4-(7-methoxy-3- quinolylamino)-2-{3- methoxy-4-[(1r,3r)-3- (dimethylamino)cyclobutoxy] phenylamino}pyrimidine |
| 338 | 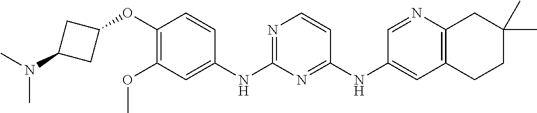 | 4-(7,7-dimethyl-5,6,7,8- tetrahydro-3-quinolylamino)- 2-{3-methoxy-4-[(1r,3r)-3- (dimethylamino)cyclobutoxy] phenylamino}pyrimidine |
| 339 | 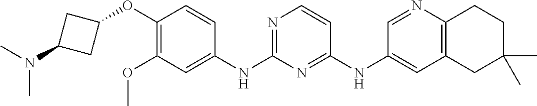 | 4-(6,6-dimethyl-5,6,7,8- tetrahydro-3-quinolylamino)- 2-{3-methoxy-4-[(1r,3r)-3- (dimethylamino)cyclobutoxy] phenylamino}pyrimidine |
| 340 | 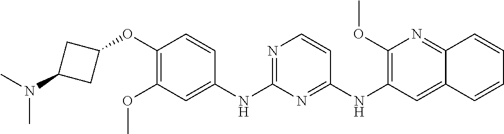 [SMILES: COc1cc(Nc2nccc(Nc3cc4ccccc4nc3OC)n2)ccc1O[C@H]1C[C@H](N(C)C)C1] | 4-(2-methoxy-3- quinolylamino)-2-{3- methoxy-4-[(1r,3r)-3- (dimethylamino)cyclobutoxy] phenylamino}pyrimidine |
| 341 | 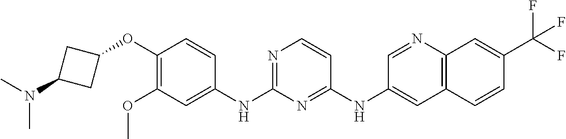 [SMILES: COc1cc(Nc2nccc(Nc3cnc4cc(C(F)(F)F)ccc4c3)n2)ccc1O[C@H]1C[C@H](N(C)C)C1] | 2-{3-methoxy-4-[(1r,3r)-3- (dimethylamino)cyclobutoxy] phenylamino}-4-[7- (trifluoromethyl)-3- quinolylamino]pyrimidine |
| 342 | 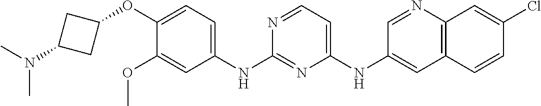 [SMILES: COc1cc(Nc2nccc(Nc3cnc4cc(Cl)ccc4c3)n2)ccc1O[C@H]1C[C@@H](N(C)C)C1] | 4-(7-chloro-3-quinolylamino)- 2-{3-methoxy-4-[(1s,3s)-3- (dimethylamino)cyclobutoxy] phenylamino}pyrimidine |
| 343 | 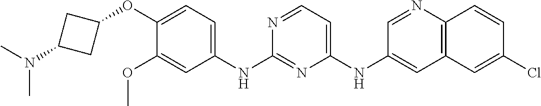 [SMILES: COc1cc(Nc2nccc(Nc3cnc4ccc(Cl)cc4c3)n2)ccc1O[C@H]1C[C@@H](N(C)C)C1] | 4-(6-chloro-3-quinolylamino)- 2-{3-methoxy-4-[(1s,3s)-3- (dimethylamino)cyclobutoxy] phenylamino}pyrimidine |
| 344 | 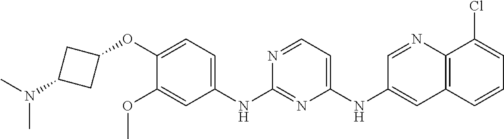 [SMILES: COc1cc(Nc2nccc(Nc3cnc4c(Cl)cccc4c3)n2)ccc1O[C@H]1C[C@@H](N(C)C)C1] | 4-(8-chloro-3-quinolylamino)- 2-{3-methoxy-4-[(1s,3s)-3- (dimethylamino)cyclobutoxy] phenylamino}pyrimidine |
| 345 | 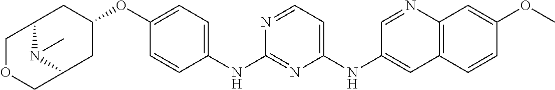 [SMILES: COc1ccc2cc(Nc3ccnc(Nc4ccc(O[C@@H]5C[C@H]6COC[C@@H](C5)N6C)cc4)n3)cnc2c1] | 2-(p-{(1R,5S,7s)-9-methyl-3- oxa-9-azabicyclo[3.3.1]non-7- yloxy} phenylamino)-4-(7- methoxy-3- quinolylamino)pyrimidine |
| 346 | 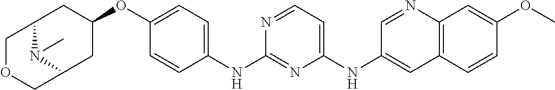 [SMILES: COc1ccc2cc(Nc3ccnc(Nc4ccc(O[C@H]5C[C@H]6COC[C@@H](C5)N6C)cc4)n3)cnc2c1] | 2-(p-{(1R,5S,7r)-9-methyl-3- oxa-9-azabicyclo[3.3.1]non-7- yloxy}phenylamino)-4-(7- methoxy-3- quinolylamino)pyrimidine |
| 347 | 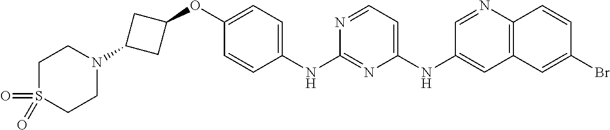 [SMILES: O=S1(=O)CCN([C@H]2C[C@H](Oc3ccc(Nc4nccc(Nc5cnc6ccc(Br)cc6c5)n4)cc3)C2)CC1] | 4-[(1r,3r)-3-{p-[4-(6-bromo- 3-quinolylamino)-2- pyrimidinylaminophenoxy} cyclobutyl]-1λ⁶,4-thiazinane- 1,1-dione |
| 348 | 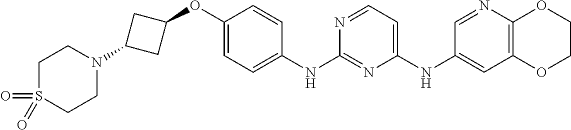 [SMILES: O=S1(=O)CCN([C@H]2C[C@H](Oc3ccc(Nc4nccc(Nc5cnc6c(c5)OCCO6)n4)cc3)C2)CC1] | 4-[(1r,3r)-3-{p-[4-(2,3- dihydro-1,4-dioxa-5-aza-7- naphthylamino)-2- pyrimidinylaminophenoxy} cyclobutyl]-1λ⁶,4-thiazinane- 1,1-dione |
| 349 | 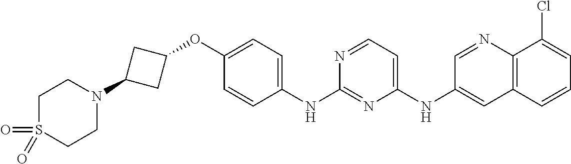 [SMILES: O=S1(=O)CCN([C@H]2C[C@H](Oc3ccc(Nc4nccc(Nc5cnc6c(Cl)cccc6c5)n4)cc3)C2)CC1] | 4-[(1r,3r)-3-{p-[4-(8-chloro-3- quinolylamino)-2- pyrimidinylaminophenoxy} cyclobutyl]-1λ⁶,4-thiazinane- 1,1-dione |
| 350 | 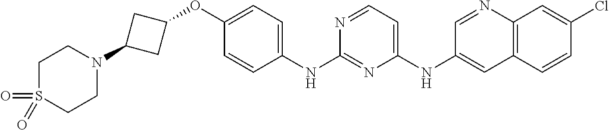 [SMILES: O=S1(=O)CCN([C@H]2C[C@H](Oc3ccc(Nc4nccc(Nc5cnc6cc(Cl)ccc6c5)n4)cc3)C2)CC1] | 4-[(1r,3r)-3-{p-[4-(7-chloro-3- quinolylamino)-2- pyrimidinylamino]phenoxy} cyclobutyl]-1λ⁶,4-thiazinane- 1,1-dione |
| 351 | 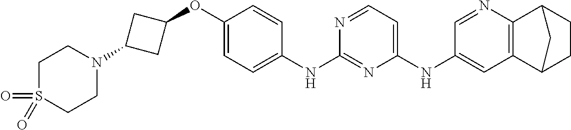 [SMILES: O=S1(=O)CCN([C@H]2C[C@H](Oc3ccc(Nc4nccc(Nc5cnc6c(c5)C5CCC6C5)n4)cc3)C2)CC1] | 4-[(1r,3r)-3-[p-(4-{3- azatricyclo[6.2.1.02,7]undeca- 2(7),3,5-trien-5-ylamino}-2- pyrimidinylamino)phenoxy] cyclobutyl]-1λ⁶,4-thiazinane- 1,1-dione |
| 352 | 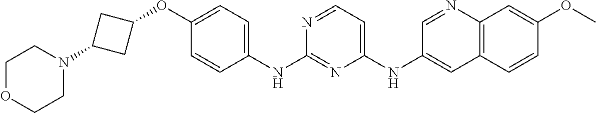 [SMILES: COc1ccc2cc(Nc3ccnc(Nc4ccc(O[C@H]5C[C@@H](N6CCOCC6)C5)cc4)n3)cnc2c1] | 4-(7-methoxy-3- quinolylamino)-2-{p-[(1s,3s)- 3- morpholinocyclobutoxy] phenylamino}pyrimidine |
| 353 | 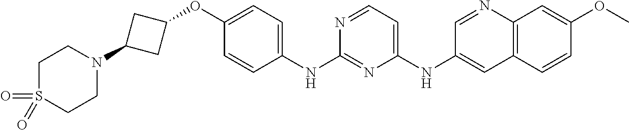 [SMILES: COc1ccc2cc(Nc3ccnc(Nc4ccc(O[C@H]5C[C@H](N6CCS(=O)(=O)CC6)C5)cc4)n3)cnc2c1] | 4-[(1r,3r)-3-{p-[4-(7- methoxy-3-quinolylamino)-2- pyrimidinylaminophenoxy} cyclobutyl]-1λ⁶,4-thiazinane- 1,1-dione |
| 354 | 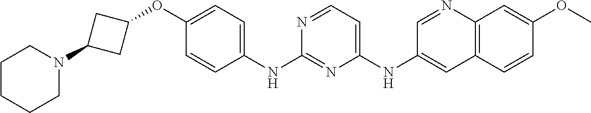 [SMILES: COc1ccc2cc(Nc3ccnc(Nc4ccc(O[C@H]5C[C@H](N6CCCCC6)C5)cc4)n3)cnc2c1] | 4-(7-methoxy-3- quinolylamino)-2-{p-[(1r,3r)- 3- piperidinocyclobutoxy]phenyl amino}pyrimidine |
| 355 | 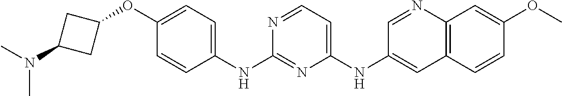 | 4-(7-methoxy-3- quinolylamino)-2-{p-[(1r,3r)- 3- (dimethylamino)cyclobutoxy] phenylamino}pyrimidine |
| 356 | 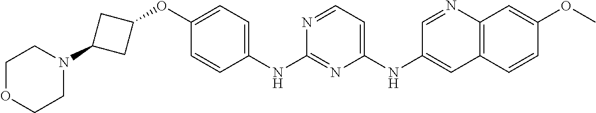 [SMILES: COc1ccc2cc(Nc3ccnc(Nc4ccc(O[C@H]5C[C@H](N6CCOCC6)C5)cc4)n3)cnc2c1] | 4-(7-methoxy-3- quinolylamino)-2-{p-[(1r,3r)-3- morpholinocyclobutoxy] phenylamino}pyrimidine |
| 357 | 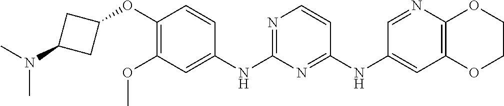 [SMILES: COc1cc(Nc2nccc(Nc3cnc4c(c3)OCCO4)n2)ccc1O[C@H]1C[C@H](N(C)C)C1] | 4-(2,3-dihydro-1,4-dioxa-5- aza-7-naphthylamino)-2-{3- methoxy-4-[(1r,3r)-3- (dimethylamino)cyclobutoxy] phenylamino}pyrimidine |
| 358 | 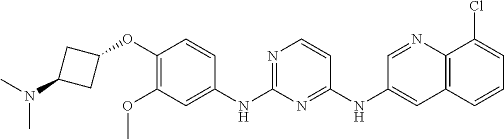 [SMILES: COc1cc(Nc2nccc(Nc3cnc4c(Cl)cccc4c3)n2)ccc1O[C@H]1C[C@H](N(C)C)C1] | 4-(8-chloro-3-quinolylamino)- 2-{3-methoxy-4-[(1r,3r)-3- (dimethylamino)cyclobutoxy] phenylamino}pyrimidine |
| 359 | 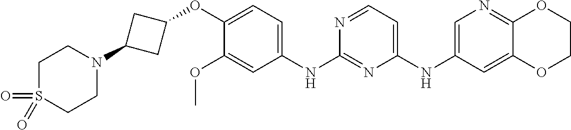 [SMILES: COc1cc(Nc2nccc(Nc3cnc4c(c3)OCCO4)n2)ccc1O[C@H]1C[C@H](N2CCS(=O)(=O)CC2)C1] | 4-[(1r,3r)-3-{4-[4-(2,3- dihydro-1,4-dioxa-5-aza-7- naphthylamino)-2- pyrimidinylamino]-2- methoxyphenoxy}cyclobutyl]- 1λ⁶,4-thiazinane-1,1-dione |
| 360 | 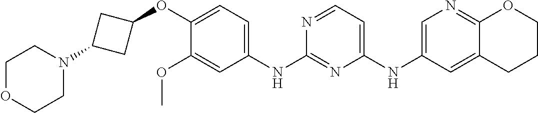 [SMILES: COc1cc(Nc2nccc(Nc3cnc4c(c3)CCCO4)n2)ccc1O[C@H]1C[C@H](N2CCOCC2)C1] | 4-(3,4-dihydro-2H-1-oxa-8- azanaphth-6-ylamino)-2-{3- methoxy-4-[(1r,3r)-3- morpholinocyclobutoxy] phenylamino}pyrimidine |
| 361 | 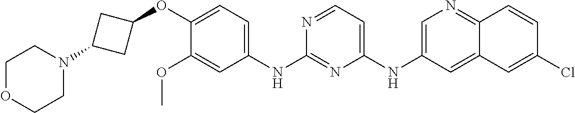 [SMILES: COc1cc(Nc2nccc(Nc3cnc4ccc(Cl)cc4c3)n2)ccc1O[C@H]1C[C@H](N2CCOCC2)C1] | 4-(6-chloro-3-quinolylamino)- 2-{3-methoxy-4-[(1r,3r)-3- morpholinocyclobutoxy] phenylamino}pyrimidine |
| 362 | 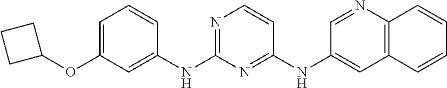 [SMILES: c1cc(Nc2nccc(Nc3cnc4ccccc4c3)n2)cc(OC2CCC2)c1] | 2-(m- cyclobutoxyphenylamino)-4- (3-quinolylamino)pyrimidine |
| 363 | 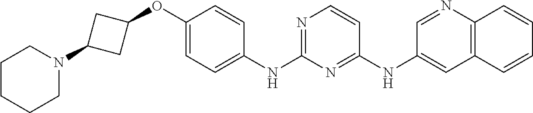 [SMILES: c1ccc2ncc(Nc3ccnc(Nc4ccc(O[C@H]5C[C@@H](N6CCCCC6)C5)cc4)n3)cc2c1] | 4-(3-quinolylamino)-2-{p- [(1s,3s)-3- piperidinocyclobutoxy]phenyl amino}pyrimidine |
| 364 | 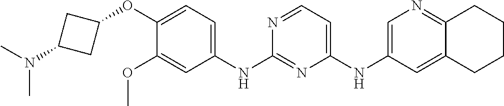 [SMILES: COc1cc(Nc2nccc(Nc3cnc4c(c3)CCCC4)n2)ccc1O[C@H]1C[C@@H](N(C)C)C1] | 2-{3-methoxy-4-[(1s,3s)-3- (dimethylamino)cyclobutoxy] phenylamino}-4-(5,6,7,8- tetrahydro-3- quinolylamino)pyrimidine |
| 365 | 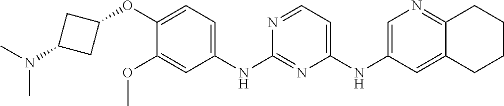 [SMILES: COc1cc(Nc2nccc(Nc3cnc4c(c3)CCCC4)n2)ccc1O[C@H]1C[C@@H](N(C)C)C1] | 4-(3,4-dihydro-2H-1-oxa-8- azanaphth-6-ylamino)-2-{3- methoxy-4-[(1s,3s)-3- (dimethylamino)cyclobutoxy] phenylamino}pyrimidine |
| 366 | 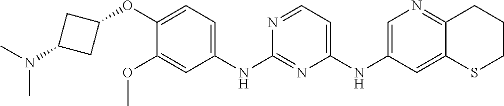 [SMILES: COc1cc(Nc2nccc(Nc3cnc4c(c3)SCCC4)n2)ccc1O[C@H]1C[C@@H](N(C)C)C1] | 4-(3,4-dihydro-2H-1-thia-5- azanaphth-7-ylamino)-2-{3- methoxy-4-[(1s,3s)-3- (dimethylamino)cyclobutoxy] phenylamino}pyrimidine |
| 367 | 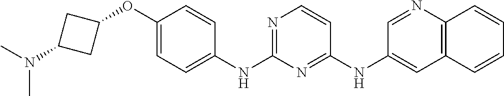 [SMILES: CN(C)[C@H]1C[C@@H](Oc2ccc(Nc3nccc(Nc4cnc5ccccc5c4)n3)cc2)C1] | 4-(3-quinolylamino)-2-{p- [(1s,3s)-3- (dimethylamino)cyclobutoxy] phenylamino}pyrimidine |
| 368 | 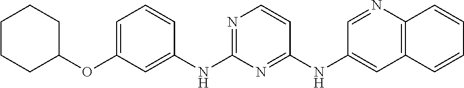 [SMILES: c1cc(Nc2nccc(Nc3cnc4ccccc4c3)n2)cc(OC2CCCCC2)c1] | 2-[m- (cyclohexyloxy)phenylamino]- 4-(3-quinolylamino)pyrimidine |
| 369 | 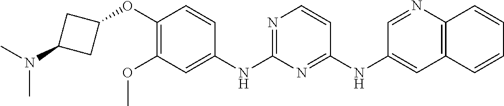 [SMILES: COc1cc(Nc2nccc(Nc3cnc4ccccc4c3)n2)ccc1O[C@H]1C[C@H](N(C)C)C1] | 2-{3-methoxy-4-[(1r,3r)-3- (dimethylamino)cyclobutoxy] phenylamino}-4-(3- quinolylamino)pyrimidine |

In some embodiments, the compound is a pharmaceutically acceptable salt of a compound described in Table 2.

TABLE 3

| Cmpd | | |
|---|---|---|
| # | Structure | Name |
| 4 | 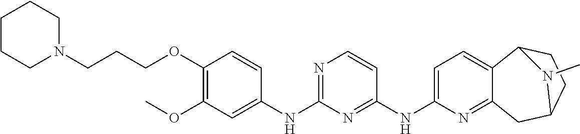 [SMILES: COc1cc(Nc2nccc(Nc3ccc4c(n3)CC3CCC4N3C)n2)ccc1OCCCN1CCCCC1] | 2-[3-methoxy-4-(3- piperidinopropoxy)phenylamino]- 4-(12-methyl-6,12- diazatricyclo[7.2.1.0^{2,7}]dodeca- 2,4,6-trien-5- ylamino)pyrimidine |
| 56 | 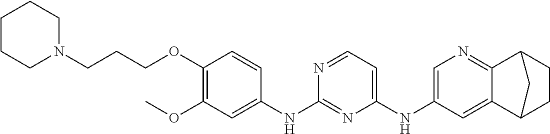 [SMILES: COc1cc(Nc2nccc(Nc3cnc4c(c3)C3CCC4C3)n2)ccc1OCCCN1CCCCC1] | 4-(3- azatricyclo[6.2.1.0^{2,7}]undeca- 2,4,6-trien-5-ylamino)-2-[3- methoxy-4-(3- piperidinopropoxy)phenylamino] pyrimidine |
| 57 | 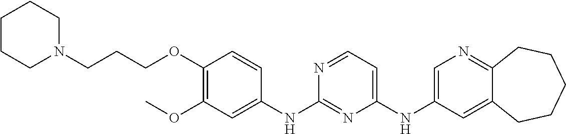 [SMILES: COc1cc(Nc2nccc(Nc3cnc4c(c3)CCCCC4)n2)ccc1OCCCN1CCCCC1] | 4-{8-azabicyclo[5.4.0]undeca- 1(11),7,9-trien-10-ylamino}-2- [3-methoxy-4-(3- piperidinopropoxy)phenylamino] pyrimidine |
| 60 | 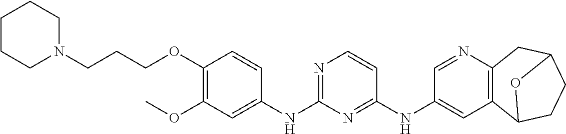 [SMILES: COc1cc(Nc2nccc(Nc3cnc4c(c3)C3CCC(C4)O3)n2)ccc1OCCCN1CCCCC1] | 2-[3-methoxy-4-(3- piperidinopropoxy)phenylamino]- 4-{12-oxa-6- azatricyclo[7.2.1.0^{2,7}]dodeca- 2(7),3,5-trien-4- ylamino}pyrimidine |
| 62 | 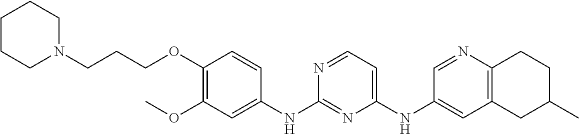 [SMILES: COc1cc(Nc2nccc(Nc3cnc4c(c3)CC(C)CC4)n2)ccc1OCCCN1CCCCC1] | 2-[3-methoxy-4-(3- piperidinopropoxy)phenylamino]- 4-(6-methyl-5,6,7,8- tetrahydro-3- quinolylamino)pyrimidine |
| 63 | 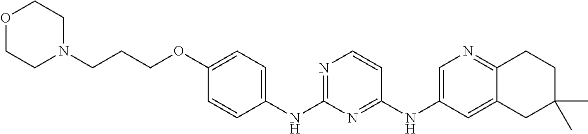 [SMILES: CC1(C)CCc2ncc(Nc3ccnc(Nc4ccc(OCCCN5CCOCC5)cc4)n3)cc2C1] | 4-(6,6-dimethyl-5,6,7,8- tetrahydro-3-quinolylamino)-2- [p-(3- morpholinopropoxy)phenylamino] pyrimidine |
| 64 | 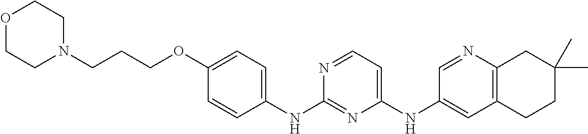 [SMILES: CC1(C)CCc2cc(Nc3ccnc(Nc4ccc(OCCCN5CCOCC5)cc4)n3)cnc2C1] | 4-(7,7-dimethyl-5,6,7,8- tetrahydro-3-quinolylamino)-2- [p-(3- morpholinopropoxy)phenylamino] pyrimidine |
| 66 | 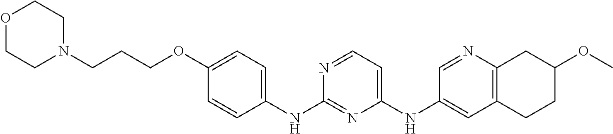 [SMILES: COC1CCc2cc(Nc3ccnc(Nc4ccc(OCCCN5CCOCC5)cc4)n3)cnc2C1] | 4-(7-methoxy-5,6,7,8- tetrahydro-3-quinolylamino)-2- [p-(3- morpholinopropoxy)phenylamino] pyrimidine |
| 67 | 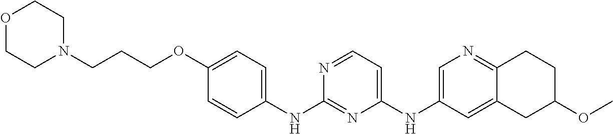 [SMILES: COC1CCc2ncc(Nc3ccnc(Nc4ccc(OCCCN5CCOCC5)cc4)n3)cc2C1] | 4-(6-methoxy-5,6,7,8- tetrahydro-3-quinolylamino)-2- [p-(3- morpholinopropoxy)phenylamino] pyrimidine |
| 68 | 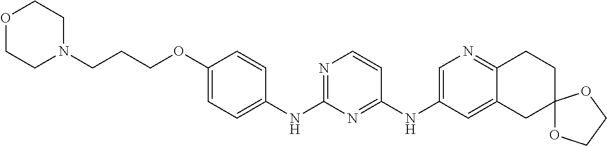 [SMILES: c1cc(Nc2cnc3c(c2)CC2(CC3)OCCO2)nc(Nc2ccc(OCCCN3CCOCC3)cc2)n1] | 4-(7′,8′-dihydro-5′H-spiro[1,3- dioxolane-2,6′-quinolin]-3′- ylamino)-2-[p-(3- morpholinopropoxy)phenylamino] pyrimidine |
| 69 | 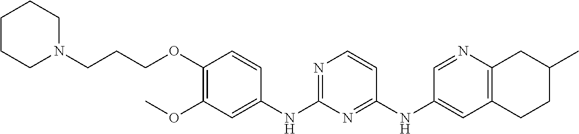 [SMILES: COc1cc(Nc2nccc(Nc3cnc4c(c3)CCC(C)C4)n2)ccc1OCCCN1CCCCC1] | 2-[3-methoxy-4-(3- piperidinopropoxy)phenylamino]- 4-(7-methyl-5,6,7,8- tetrahydro-3- quinolylamino)pyrimidine |
| 70 | 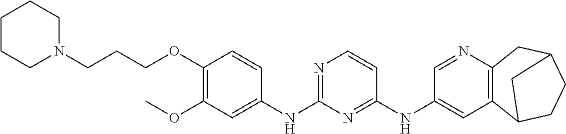 [SMILES: COc1cc(Nc2nccc(Nc3cnc4c(c3)C3CCC(C4)C3)n2)ccc1OCCCN1CCCCC1] | 4-{6- azatricyclo[7.2.1.0^{2,7}] dodeca- 2(7),3,5-trien-4-ylamino}-2-[3- methoxy-4-(3- piperidinopropoxy)phenylamino] pyrimidine |
| 71 | 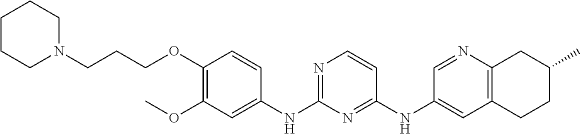 [SMILES: COc1cc(Nc2nccc(Nc3cnc4c(c3)CC[C@@H](C)C4)n2)ccc1OCCCN1CCCCC1] | 4-[(R)-7-methyl-5,6,7,8- tetrahydro-3-quinolylamino]-2- [3-methoxy-4-(3- piperidinopropoxy)phenylamino] pyrimidine |
| 73 | 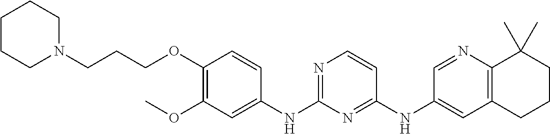 [SMILES: COc1cc(Nc2nccc(Nc3cnc4c(c3)CCCC4(C)C)n2)ccc1OCCCN1CCCCC1] | 4-(8,8-dimethyl-5,6,7,8- tetrahydro-3-quinolylamino)-2- [3-methoxy-4-(3- piperidinopropoxy)phenylamino] pyrimidine |
| 74 | 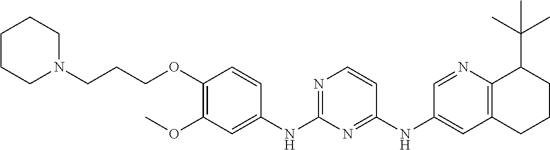 [SMILES: COc1cc(Nc2nccc(Nc3cnc4c(c3)CCCC4C(C)(C)C)n2)ccc1OCCCN1CCCCC1] | 4-[8-(tert-butyl)-5,6,7,8- tetrahydro-3-quinolylamino]-2- [3-methoxy-4-(3- piperidinopropoxy)phenylamino] pyrimidine |
| 194 | 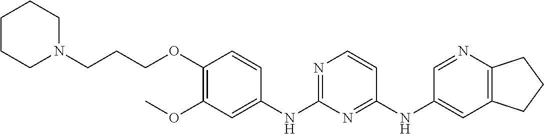 [SMILES: COc1cc(Nc2nccc(Nc3cnc4c(c3)CCC4)n2)ccc1OCCCN1CCCCC1] | 4-(4-aza-6-indanylamino)-2-[3- methoxy-4-(3- piperidinopropoxy)phenylamino] pyrimidine |
| 303 | 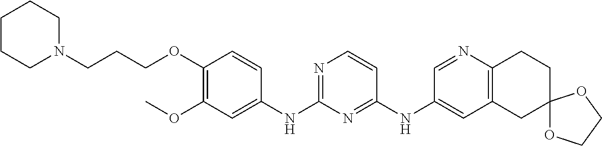 [SMILES: COc1cc(Nc2nccc(Nc3cnc4c(c3)CC3(CC4)OCCO3)n2)ccc1OCCCN1CCCCC1] | 4-(7′,8′-dihydro-5′H-spiro[1,3- dioxolane-2,6′-quinolin]-3′- ylamino)-2-[3-methoxy-4-(3- piperidinopropoxy)phenylamino] pyrimidine |
| 370 | 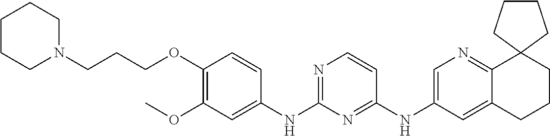 [SMILES: COc1cc(Nc2nccc(Nc3cnc4c(c3)CCCC43CCCC3)n2)ccc1OCCCN1CCCCC1] | 4-(6′,7′-dihydro-5′H- spiro[cyclopentane-1,8′- quinolin]-3′-ylamino)-2-[3- methoxy-4-(3- piperidinopropoxy)phenylamino] pyrimidine |
| 371 | 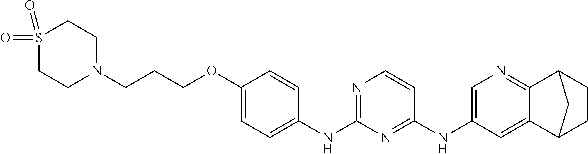 [SMILES: O=S1(=O)CCN(CCCOc2ccc(Nc3nccc(Nc4cnc5c(c4)C4CCC5C4)n3)cc2)CC1] | 4-[3-(p-{4-(3- azatricyclo[6.2.1.0^{2,7}]undeca- 2,4,6-trien-5-ylamino)-2- pyrimidinylamino}phenoxy) propyl]-1λ⁶,4- thiazinane-1,1-dione |
| 372 | 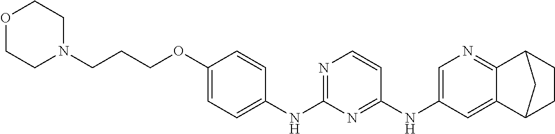 [SMILES: c1cc(Nc2cnc3c(c2)C2CCC3C2)nc(Nc2ccc(OCCCN3CCOCC3)cc2)n1] | 4-(3- azatricyclo[6.2.1.0^{2,7}]undeca- 2,4,6-trien-5-ylamino)-2-[p-(3- morpholinopropoxy) phenylamino]pyrimidine |
| 373 | 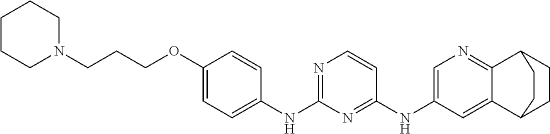 [SMILES: c1cc(Nc2cnc3c(c2)C2CCC3CC2)nc(Nc2ccc(OCCCN3CCCCC3)cc2)n1] | 4-(3- azatricyclo[6.2.2.0^{2,7}]dodeca- 2,4,6-trien-5-ylamino)-2-[p-(3- morpholinopropoxy)phenylamino] pyrimidine |
| 374 | 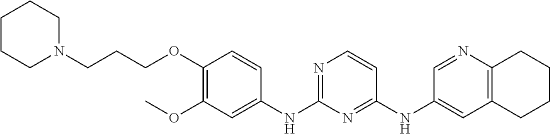 [SMILES: COc1cc(Nc2nccc(Nc3cnc4c(c3)CCCC4)n2)ccc1OCCCN1CCCCC1] | 2-[3-methoxy-4-(3- piperidinopropoxy)phenylamino]- 4-(5,6,7,8-tetrahydro-3- quinolylamino)pyrimidine |

In some embodiments, the compound is a pharmaceutically acceptable salt of a compound described in Table 3.

TABLE 4

| Cmpd | | |
|---|---|---|
| # | Structure | Name |
| 53 | 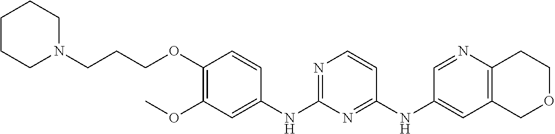 [SMILES: COc1cc(Nc2nccc(Nc3cnc4c(c3)COCC4)n2)ccc1OCCCN1CCCCC1] | 4-(7,8-dihydro-5H-6-oxa-1- azanaphth-3-ylamino)-2-[3- methoxy-4-(3- piperidinopropoxy)phenylamino] pyrimidine |
| 54 | 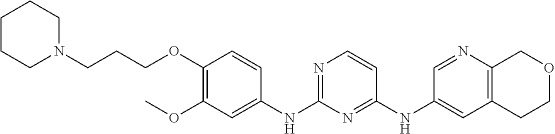 [SMILES: COc1cc(Nc2nccc(Nc3cnc4c(c3)CCOC4)n2)ccc1OCCCN1CCCCC1] | 4-(6,8-dihydro-5H-7-oxa-1- azanaphth-3-ylamino)-2-[3- methoxy-4-(3- piperidinopropoxy)phenylamino] pyrimidine |
| 61 | 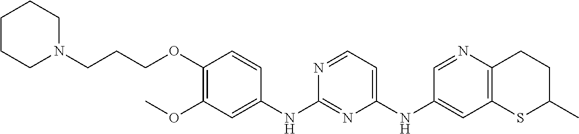 [SMILES: COc1cc(Nc2nccc(Nc3cnc4c(c3)SC(C)CC4)n2)ccc1OCCCN1CCCCC1] | 2-[3-methoxy-4-(3- piperidinopropoxy)phenylamino]- 4-(2-methyl-3,4-dihydro-2H- 1-thia-5-azanaphth-7- ylamino)pyrimidine |
| 65 | 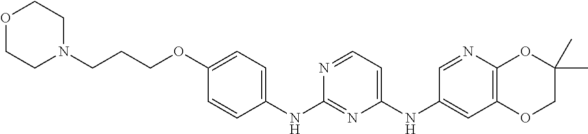 [SMILES: CC1(C)COc2cc(Nc3ccnc(Nc4ccc(OCCCN5CCOCC5)cc4)n3)cnc2O1] | 4-(3,3-dimethyl-2,3-dihydro- 1,4-dioxa-5-aza-7- naphthylamino)-2-[p-(3- morpholinopropoxy) phenylamino]pyrimidine |
| 101 | 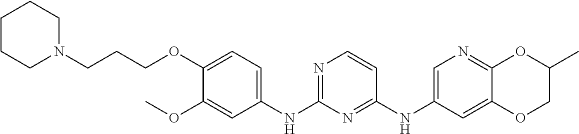 [SMILES: COc1cc(Nc2nccc(Nc3cnc4c(c3)OCC(C)O4)n2)ccc1OCCCN1CCCCC1] | 2-[3-methoxy-4-(3- piperidinopropoxy)phenylamino]- 4-(3-methyl-2,3-dihydro-1,4- dioxa-5-aza-7- naphthylamino)pyrimidine |
| 105 | 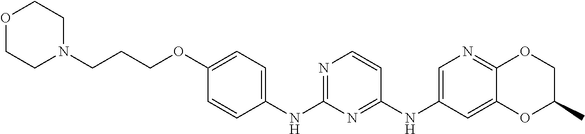 [SMILES: C[C@@H]1COc2ncc(Nc3ccnc(Nc4ccc(OCCCN5CCOCC5)cc4)n3)cc2O1] | 4-[(R)-2-methyl-2,3-dihydro- 1,4-dioxa-5-aza-7- naphthylamino]-2-[p-(3- morpholinopropoxy) phenylamino]pyrimidine |
| 106 | 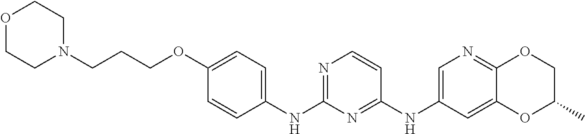 [SMILES: C[C@H]1COc2ncc(Nc3ccnc(Nc4ccc(OCCCN5CCOCC5)cc4)n3)cc2O1] | 4-[(S)-2-methyl-2,3-dihydro- 1,4-dioxa-5-aza-7- naphthy lamino]-2-[p-(3- morpholinopropoxy) phenylamino]pyrimidine |
| 201 | 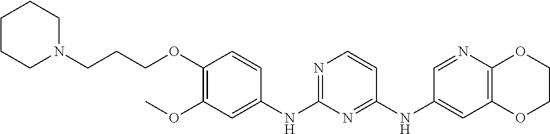 [SMILES: COc1cc(Nc2nccc(Nc3cnc4c(c3)OCCO4)n2)ccc1OCCCN1CCCCC1] | 4-(2,3-dihydro-1,4-dioxa-5-aza- 7-naphthylamino)-2-[3- methoxy-4-(3- piperidinopropoxy) phenylamino]pyrimidine |
| 375 | 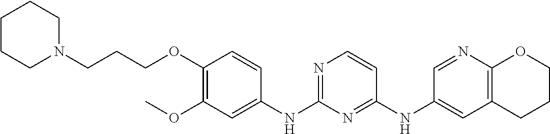 [SMILES: COc1cc(Nc2nccc(Nc3cnc4c(c3)CCCO4)n2)ccc1OCCCN1CCCCC1] | 4-(3,4-dihydro-2H-1-oxa-8- azanaphth-6-ylamino)-2-[3- methoxy-4-(3- piperidinopropoxy) phenylamino]pyrimidine |
| 376 | 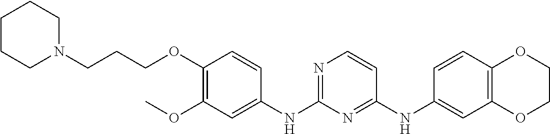 [SMILES: COc1cc(Nc2nccc(Nc3ccc4c(c3)OCCO4)n2)ccc1OCCCN1CCCCC1] | 4-(2,3-dihydro-1,4- benzodioxin-6-ylamino)-2-[3- methoxy-4-(3- piperidinopropoxy) phenylamino]pyrimidine |
| 377 | 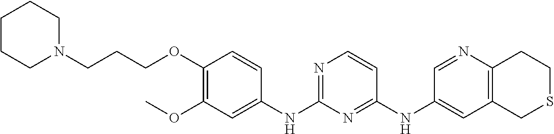 [SMILES: COc1cc(Nc2nccc(Nc3cnc4c(c3)CSCC4)n2)ccc1OCCCN1CCCCC1] | 4-(7,8-dihydro-5H-6-thia-1- azanaphth-3-ylamino)-2-[3- methoxy-4-(3- piperidinopropoxy) phenylamino]pyrimidine |
| 378 | 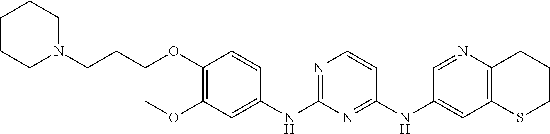 [SMILES: COc1cc(Nc2nccc(Nc3cnc4c(c3)SCCC4)n2)ccc1OCCCN1CCCCC1] | 4-(3,4-dihydro-2H-1-thia-5- azanaphth-7-ylamino)-2-[3- methoxy-4-(3- piperidinopropoxy) phenylamino]pyrimidine |
| 379 | 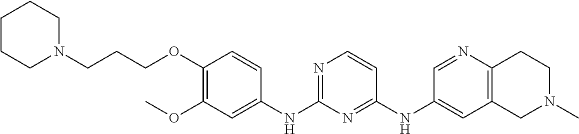 [SMILES: COc1cc(Nc2nccc(Nc3cnc4c(c3)CN(C)CC4)n2)ccc1OCCCN1CCCCC1] | 2-[3-methoxy-4-(3- piperidinopropoxy)phenylamino]- 4-(6-methyl-5,6,7,8- tetrahydro-1,6-diaza-3- naphthylamino)pyrimidine |
| 380 | 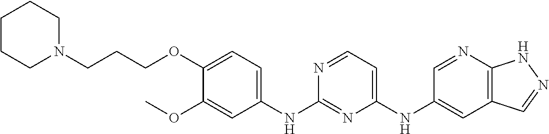 [SMILES: COc1cc(Nc2nccc(Nc3cnc4[nH]ncc4c3)n2)ccc1OCCCN1CCCCC1] | 2-[3-methoxy-4-(3- piperidinopropoxy)phenylamino]- 4-(1H-1,2,7-triazainden-5- ylamino)pyrimidine |
| 381 | 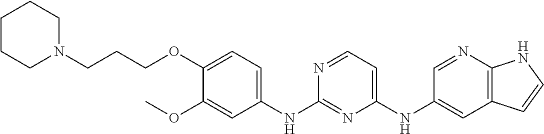 [SMILES: COc1cc(Nc2nccc(Nc3cnc4[nH]ccc4c3)n2)ccc1OCCCN1CCCCC1] | 4-(1H-1,7-diazainden-5- ylamino)-2-[3-methoxy-4-(3- piperidinopropoxy) phenylamino]pyrimidine |
| 382 | 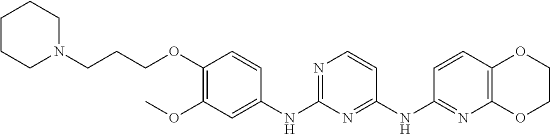 [SMILES: COc1cc(Nc2nccc(Nc3ccc4c(n3)OCCO4)n2)ccc1OCCCN1CCCCC1] | 4-(2,3-dihydro-1,4-dioxa-5-aza- 6-naphthylamino)-2-[3- methoxy-4-(3- piperidinopropoxy) phenylamino]pyrimidine |
| 383 | 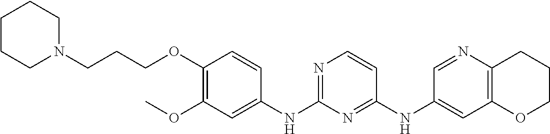 [SMILES: COc1cc(Nc2nccc(Nc3cnc4c(c3)OCCC4)n2)ccc1OCCCN1CCCCC1] | 4-(3,4-dihydro-2H-1-oxa-5- azanaphth-7-ylamino)-2-[3- methoxy-4-(3- piperidinopropoxy) phenylamino]pyrimidine |
| 384 | 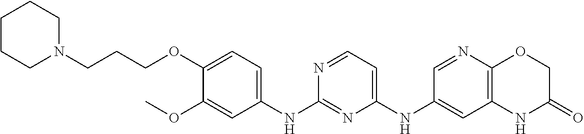 [SMILES: COc1cc(Nc2nccc(Nc3cnc4c(c3)NC(=O)CO4)n2)ccc1OCCCN1CCCCC1] | 7-{2-[3-methoxy-4-(3- piperidinopropoxy)phenylamino]- 4-pyrimidinylamino}-1,3- dihydro-4-oxa-1,5-diaza-2- naphthalenone |
| 385 | 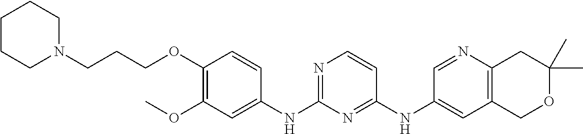 [SMILES: COc1cc(Nc2nccc(Nc3cnc4c(c3)COC(C)(C)C4)n2)ccc1OCCCN1CCCCC1] | 4-(7,7-dimethyl-7,8-dihydro- 5H-6-oxa-1-azanaphth-3- ylamino)-2-[3-methoxy-4-(3- piperidinopropoxy) phenylamino]pyrimidine |
| 386 | 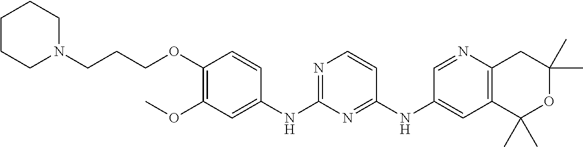 [SMILES: COc1cc(Nc2nccc(Nc3cnc4c(c3)C(C)(C)OC(C)(C)C4)n2)ccc1OCCCN1CCCCC1] | 2-[3-methoxy-4-(3- piperidinopropoxy)phenylamino]- 4-(5,5,7,7-tetramethyl-7,8- dihydro-5H-6-oxa-1-azanaphth- 3-ylamino)pyrimidine |
| 387 | 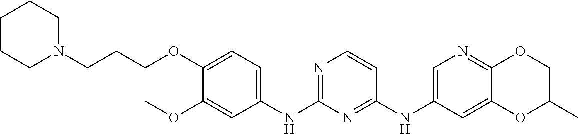 [SMILES: COc1cc(Nc2nccc(Nc3cnc4c(c3)OC(C)CO4)n2)ccc1OCCCN1CCCCC1] | 2-[3-methoxy-4-(3- piperidinopropoxy)phenylamino]- 4-(2-methyl-2,3-dihydro-1,4- dioxa-5-aza-7- naphthylamino)pyrimidine |
| 388 | 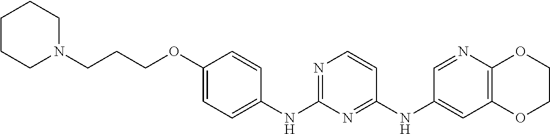 [SMILES: c1cc(Nc2cnc3c(c2)OCCO3)nc(Nc2ccc(OCCCN3CCCCC3)cc2)n1] | 4-(2,3-dihydro-1,4-dioxa-5-aza- 7-naphthylamino)-2-[p-(3- morpholinopropoxy) phenylamino]pyrimidine |
| 389 | 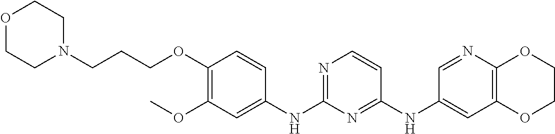 [SMILES: COc1cc(Nc2nccc(Nc3cnc4c(c3)OCCO4)n2)ccc1OCCCN1CCOCC1] | 4-(2,3-dihydro-1,4-dioxa-5-aza- 7-naphthylamino)-2-[3- methoxy-4-(3- morpholinopropoxy) phenylamino]pyrimidine |
| 390 | 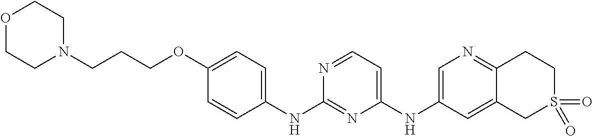 [SMILES: O=S1(=O)CCc2ncc(Nc3ccnc(Nc4ccc(OCCCN5CCOCC5)cc4)n3)cc2C1] | 3-{2-[p-(3- morpholinopropoxy)phenylamino]- 4-pyrimidinylamino}- 5,6,7,8-tetrahydro-6H,6H-6λ⁶- thia-1-azanaphthalene-6,6-dione |
| 391 | 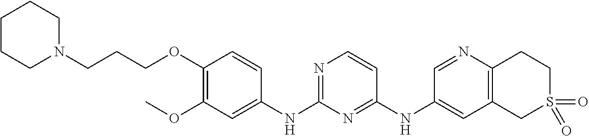 [SMILES: COc1cc(Nc2nccc(Nc3cnc4c(c3)CS(=O)(=O)CC4)n2)ccc1OCCCN1CCCCC1] | 3-{2-[3-methoxy-4-(3- piperidinopropoxy)phenylamino]- 4-pyrimidinylamino}- 5,6,7,8-tetrahydro-6H,6H-6λ⁶- thia-1-azanaphthalene-6,6-dione |
| 506 | 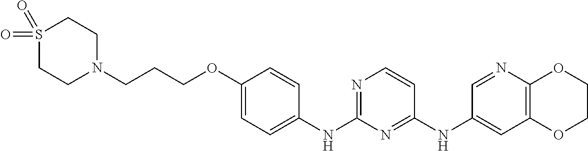 [SMILES: O=S1(=O)CCN(CCCOc2ccc(Nc3nccc(Nc4cnc5c(c4)OCCO5)n3)cc2)CC1] | 4-(3-{p-[4-(2,3-dihydro-1,4- dioxa-5-aza-7-naphthylamino)- 2- pyrimidinylamino]phenoxy} propyl)-1λ⁶,4-thiazinane-1,1-dione |

In some embodiments, the compound is a pharmaceutically acceptable salt of a compound described in Table 4.

TABLE 5

| Cmpd | | |
|---|---|---|
| # | Structure | Name |
| 5 | 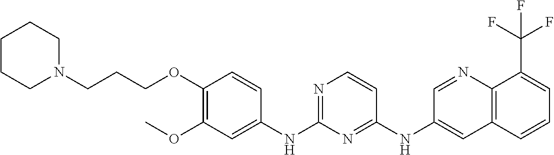 [SMILES: COc1cc(Nc2nccc(Nc3cnc4c(C(F)(F)F)cccc4c3)n2)ccc1OCCCN1CCCCC1] | 2-[3-methoxy-4-(3- piperidinopropoxy) phenylamino]-4-[8- (trifluoromethyl)-3- quinolylamino]pyrimidine |
| 6 | 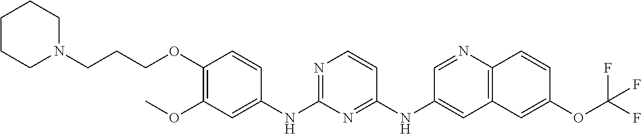 [SMILES: COc1cc(Nc2nccc(Nc3cnc4ccc(OC(F)(F)F)cc4c3)n2)ccc1OCCCN1CCCCC1] | 2-[3-methoxy-4-(3- piperidinopropoxy) phenylamino]-4-(6- trifluoromethoxy-3- quinolylamino)pyrimidine |
| 13 | 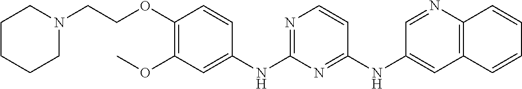 [SMILES: COc1cc(Nc2nccc(Nc3cnc4ccccc4c3)n2)ccc1OCCN1CCCCC1] | 2-[3-methoxy-4-(2- piperidinoethoxy) phenylamino]-4-(3- quinolylamino)pyrimidine |
| 14 | 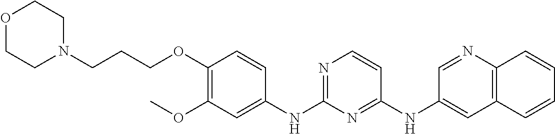 [SMILES: COc1cc(Nc2nccc(Nc3cnc4ccccc4c3)n2)ccc1OCCCN1CCOCC1] | 2-[3-methoxy-4-(3- morpholinopropoxy) phenylamino]-4-(3- quinolylamino)pyrimidine |
| 20 | 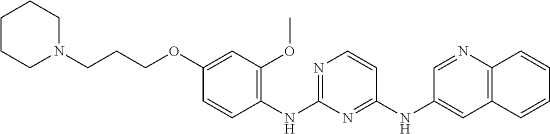 [SMILES: COc1cc(OCCCN2CCCCC2)ccc1Nc1nccc(Nc2cnc3ccccc3c2)n1] | 2-[2-methoxy-4-(3- piperidinopropoxy) phenylamino]-4-(3- quinolylamino)pyrimidine |
| 31 | 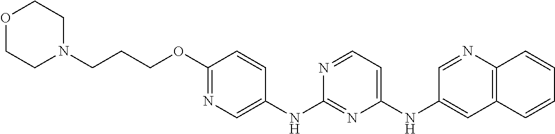 [SMILES: c1ccc2ncc(Nc3ccnc(Nc4ccc(OCCCN5CCOCC5)nc4)n3)cc2c1] | 2-[6-(3- morpholinopropoxy)-3- pyridylamino]-4-(3- quinolylamino)pyrimidine |
| 72 | 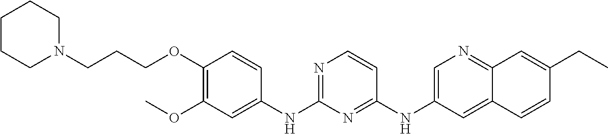 [SMILES: CCc1ccc2cc(Nc3ccnc(Nc4ccc(OCCCN5CCCCC5)c(OC)c4)n3)cnc2c1] | 4-(7-ethyl-3-quinolylamino)- 2-[3-methoxy-4-(3- piperidinopropoxy) phenylamino]pyrimidine |
| 96 | 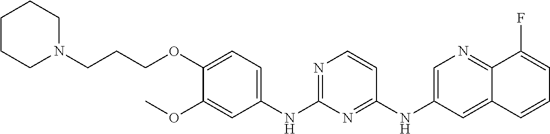 [SMILES: COc1cc(Nc2nccc(Nc3cnc4c(F)cccc4c3)n2)ccc1OCCCN1CCCCC1] | 4-(8-fluoro-3- quinolylamino)-2-[3- methoxy-4-(3- piperidinopropoxy) phenylamino]pyrimidine |
| 97 | 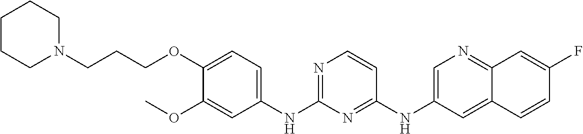 [SMILES: COc1cc(Nc2nccc(Nc3cnc4cc(F)ccc4c3)n2)ccc1OCCCN1CCCCC1] | 4-(7-fluoro-3- quinolylamino)-2-[3- methoxy-4-(3- piperidinopropoxy) phenylamino]pyrimidine |
| 98 | 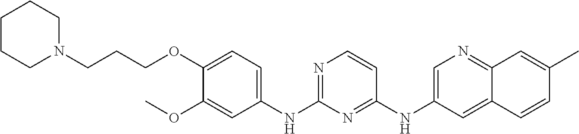 [SMILES: COc1cc(Nc2nccc(Nc3cnc4cc(C)ccc4c3)n2)ccc1OCCCN1CCCCC1] | 2-[3-methoxy-4-(3- piperidinopropoxy) phenylamino]- 4-(7-methyl-3- quinolylamino)pyrimidine |
| 99 | 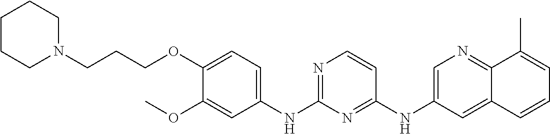 [SMILES: COc1cc(Nc2nccc(Nc3cnc4c(C)cccc4c3)n2)ccc1OCCCN1CCCCC1] | 2-[3-methoxy-4-(3- piperidinopropoxy) phenylamino]- 4-(8-methyl-3- quinolylamino)pyrimidine |
| 102 | 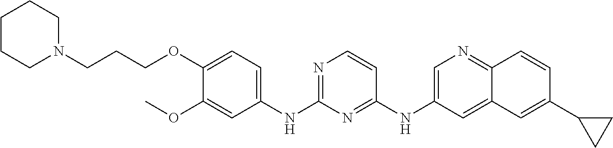 [SMILES: COc1cc(Nc2nccc(Nc3cnc4ccc(C5CC5)cc4c3)n2)ccc1OCCCN1CCCCC1] | 4-(6-cyclopropyl-3- quinolylamino)-2-[3- methoxy-4-(3- piperidinopropoxy) phenylamino]pyrimidine |
| 103 | 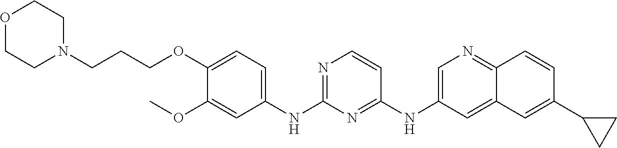 [SMILES: COc1cc(Nc2nccc(Nc3cnc4ccc(C5CC5)cc4c3)n2)ccc1OCCCN1CCOCC1] | 4-(6-cyclopropyl-3- quinolylamino)-2-[3- methoxy-4-(3- morpholinopropoxy) phenylamino]pyrimidine |
| 104 | 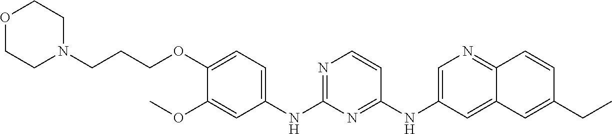 [SMILES: CCc1ccc2ncc(Nc3ccnc(Nc4ccc(OCCCN5CCOCC5)c(OC)c4)n3)cc2c1] | 4-(6-ethyl-3-quinolylamino)- 2-[3-methoxy-4-(3- morpholinopropoxy) phenylamino]pyrimidine |
| 107 | 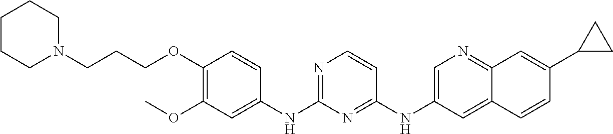 [SMILES: COc1cc(Nc2nccc(Nc3cnc4cc(C5CC5)ccc4c3)n2)ccc1OCCCN1CCCCC1] | 4-(7-cyclopropyl-3- quinolylamino)-2-[3- methoxy-4-(3- piperidinopropoxy) phenylamino]pyrimidine |
| 108 | 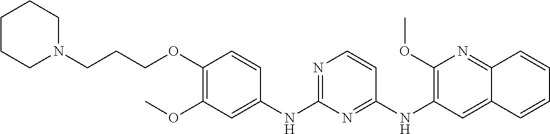 [SMILES: COc1cc(Nc2nccc(Nc3cc4ccccc4nc3OC)n2)ccc1OCCCN1CCCCC1] | 2-[3-methoxy-4-(3- piperidinopropoxy) phenylamino]- 4-(2-methoxy-3- quinolylamino)pyrimidine |
| 196 | 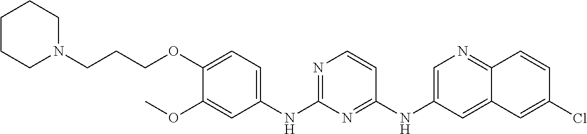 [SMILES: COc1cc(Nc2nccc(Nc3cnc4ccc(Cl)cc4c3)n2)ccc1OCCCN1CCCCC1] | 4-(6-chloro-3- quinolylamino)-2-[3- methoxy-4-(3- piperidinopropoxy) phenylamino]pyrimidine |
| 206 | 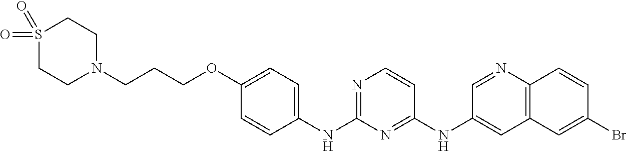 [SMILES: O=S1(=O)CCN(CCCOc2ccc(Nc3nccc(Nc4cnc5ccc(Br)cc5c4)n3)cc2)CC1] | 4-(3-{p-[4-(6-bromo-3- quinolylamino)-2- pyrimidinylamino]phenoxy} propyl)-1λ⁶,4-thiazinane-1,1- dione |
| 392 | 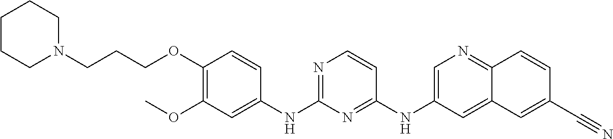 [SMILES: COc1cc(Nc2nccc(Nc3cnc4ccc(C#N)cc4c3)n2)ccc1OCCCN1CCCCC1] | 3-{2-[3-methoxy-4-(3- piperidinopropoxy) phenylamino]-4- pyrimidiny lamino}- 6-quinolinecarbonitrile |
| 393 | 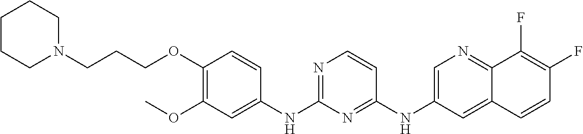 [SMILES: COc1cc(Nc2nccc(Nc3cnc4c(F)c(F)ccc4c3)n2)ccc1OCCCN1CCCCC1] | 4-(7,8-difluoro-3- quinolylamino)-2-[3- methoxy-4-(3- piperidinopropoxy) phenylamino]pyrimidine |
| 394 | 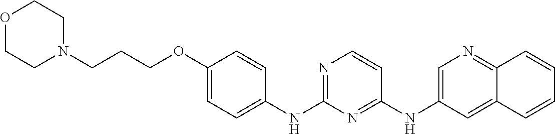 [SMILES: c1ccc2ncc(Nc3ccnc(Nc4ccc(OCCCN5CCOCC5)cc4)n3)cc2c1] | 2-[p-(3- morpholinopropoxy) phenylamino]-4-(3- quinolylamino)pyrimidine |
| 395 | 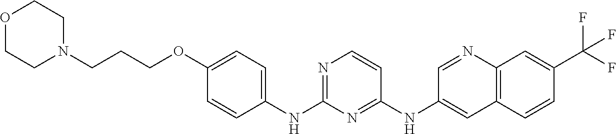 [SMILES: FC(F)(F)c1ccc2cc(Nc3ccnc(Nc4ccc(OCCCN5CCOCC5)cc4)n3)cnc2c1] | 2-[p-(3- morpholinopropoxy) phenylamino]-4-[7- (trifluoromethyl)-3- quinolylamino]pyrimidine |
| 396 | 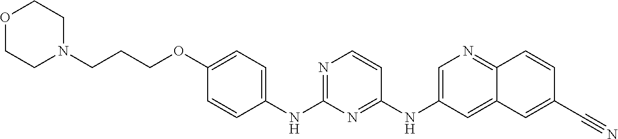 [SMILES: N#Cc1ccc2ncc(Nc3ccnc(Nc4ccc(OCCCN5CCOCC5)cc4)n3)cc2c1] | 3-{2-[p-(3- morpholinopropoxy) phenylamino]-4- pyrimidinylamino}- 6-quinolinecarbonitrile |
| 397 | 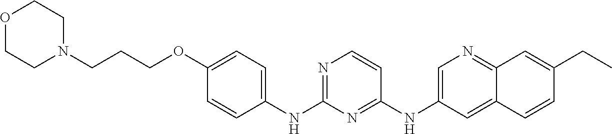 [SMILES: CCc1ccc2cc(Nc3ccnc(Nc4ccc(OCCCN5CCOCC5)cc4)n3)cnc2c1] | 4-(7-ethyl-3-quinolylamino)- 2-[p-(3- morpholinopropoxy) phenylamino]pyrimidine |
| 398 | 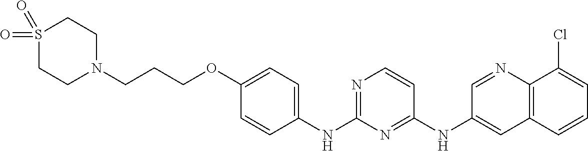 [SMILES: O=S1(=O)CCN(CCCOc2ccc(Nc3nccc(Nc4cnc5c(Cl)cccc5c4)n3)cc2)CC1] | 4-(3-{p-[4-(8-chloro-3- quinolylamino)-2- pyrimidinylamino]phenoxy} propyl)-1λ⁶,4-thiazinane-1,1- dione |
| 399 | 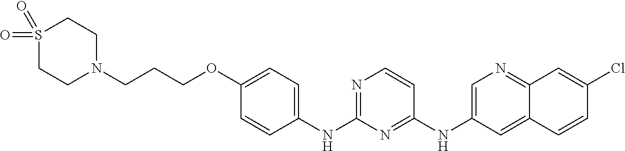 [SMILES: O=S1(=O)CCN(CCCOc2ccc(Nc3nccc(Nc4cnc5cc(Cl)ccc5c4)n3)cc2)CC1] | 4-(3-{p-[4-(7-chloro-3- quinolylamino)-2- pyrimidinylamino]phenoxy} propyl)-1λ⁶,4-thiazinane-1,1- dione |
| 400 | 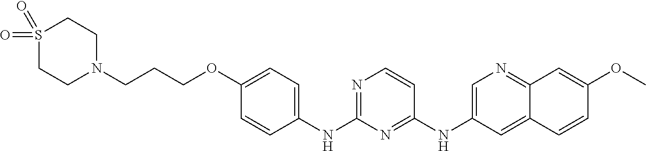 [SMILES: COc1ccc2cc(Nc3ccnc(Nc4ccc(OCCCN5CCS(=O)(=O)CC5)cc4)n3)cnc2c1] | 4-(3-{p-[4-(7-methoxy-3- quinolylamino)-2- pyrimidinylamino]phenoxy} propyl)-1λ⁶,4-thiazinane-1,1- dione |
| 401 | 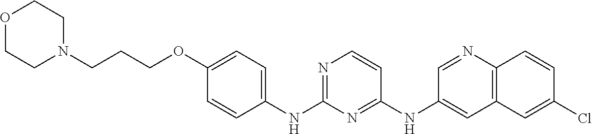 [SMILES: Clc1ccc2ncc(Nc3ccnc(Nc4ccc(OCCCN5CCOCC5)cc4)n3)cc2c1] | 4-(6-chloro-3- quinolylamino)-2-[p-(3- morpholinopropoxy) phenylamino]pyrimidine |
| 402 | 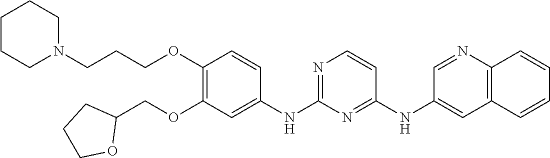 [SMILES: c1ccc2ncc(Nc3ccnc(Nc4ccc(OCCCN5CCCCC5)c(OCC5CCCO5)c4)n3)cc2c1] | 2-{4-(3-piperidinopropoxy)- 3-[(tetrahydro-2- furyl)methoxy]phenylamino}- 4-(3- quinolylamino)pyrimidine |
| 403 | 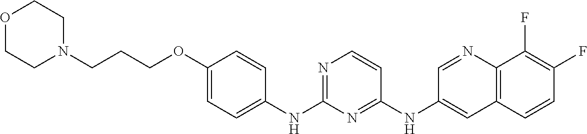 [SMILES: Fc1ccc2cc(Nc3ccnc(Nc4ccc(OCCCN5CCOCC5)cc4)n3)cnc2c1F] | 4-(7,8-difluoro-3- quinolylamino)-2-[p-(3- morpholinopropoxy) phenylamino]pyrimidine |
| 404 | 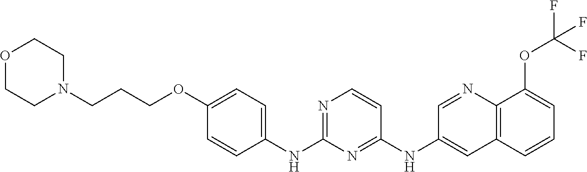 [SMILES: FC(F)(F)Oc1cccc2cc(Nc3ccnc(Nc4ccc(OCCCN5CCOCC5)cc4)n3)cnc12] | 2-[p-(3- morpholinopropoxy) phenylamino]-4-(8- trifluoromethoxy-3- quinolylamino)pyrimidine |
| 405 | 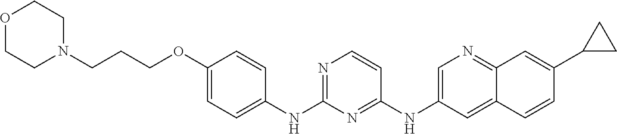 [SMILES: c1cc(Nc2cnc3cc(C4CC4)ccc3c2)nc(Nc2ccc(OCCCN3CCOCC3)cc2)n1] | 4-(7-cyclopropyl-3- quinolylamino)-2-[p-(3- morpholinopropoxy) phenylamino]pyrimidine |
| 406 | 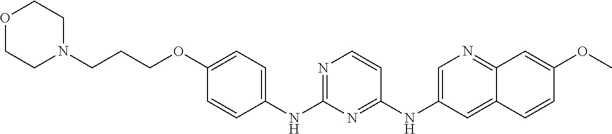 [SMILES: COc1ccc2cc(Nc3ccnc(Nc4ccc(OCCCN5CCOCC5)cc4)n3)cnc2c1] | 4-(7-methoxy-3- quinolylamino)-2-[p-(3- morpholinopropoxy) phenylamino]pyrimidine |
| 407 | 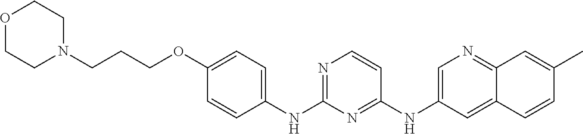 [SMILES: Cc1ccc2cc(Nc3ccnc(Nc4ccc(OCCCN5CCOCC5)cc4)n3)cnc2c1] | 4-(7-methyl-3- quinolylamino)-2-[p-(3- morpholinopropoxy) phenylamino]pyrimidine |
| 408 | 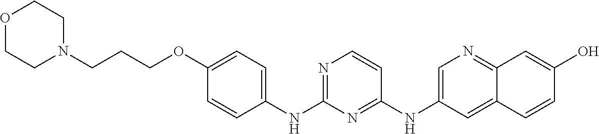 [SMILES: Oc1ccc2cc(Nc3ccnc(Nc4ccc(OCCCN5CCOCC5)cc4)n3)cnc2c1] | 3-{2-[p-(3- morpholinopropoxy) phenylamino]-4- pyrimidinylamino}- 7-quinolinol |
| 409 | 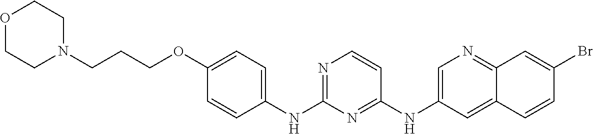 [SMILES: Brc1ccc2cc(Nc3ccnc(Nc4ccc(OCCCN5CCOCC5)cc4)n3)cnc2c1] | 4-(7-bromo-3- quinolylamino)-2-[p-(3- morpholinopropoxy) phenylamino]pyrimidine |
| 410 | 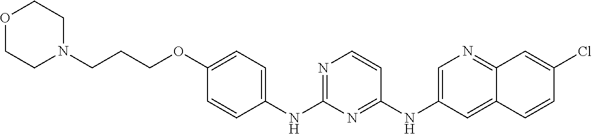 [SMILES: Clc1ccc2cc(Nc3ccnc(Nc4ccc(OCCCN5CCOCC5)cc4)n3)cnc2c1] | 4-(7-chloro-3- quinolylamino)-2-[p-(3- morpholinopropoxy) phenylamino]pyrimidine |
| 411 | 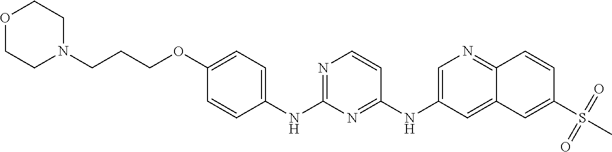 [SMILES: CS(=O)(=O)c1ccc2ncc(Nc3ccnc(Nc4ccc(OCCCN5CCOCC5)cc4)n3)cc2c1] | 4-(6-mesyl-3- quinolylamino)-2-[p-(3- morpholinopropoxy) phenylamino]pyrimidine |
| 412 | 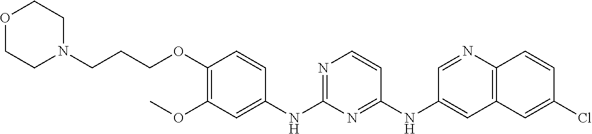 [SMILES: COc1cc(Nc2nccc(Nc3cnc4ccc(Cl)cc4c3)n2)ccc1OCCCN1CCOCC1] | 2-[3-methoxy-4-(3- piperidinopropoxy) phenylamino]- 4-(2-methyl-3,4- dihydro-2H-1-thia-5- azanaphth-7- ylamino)pyrimidine |
| 413 | 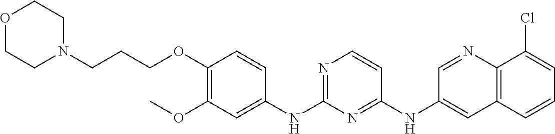 [SMILES: COc1cc(Nc2nccc(Nc3cnc4c(Cl)cccc4c3)n2)ccc1OCCCN1CCOCC1] | 4-(8-chloro-3- quinolylamino)-2-[3- methoxy-4-(3- morpholinopropoxy) phenylamino]pyrimidine |
| 414 | 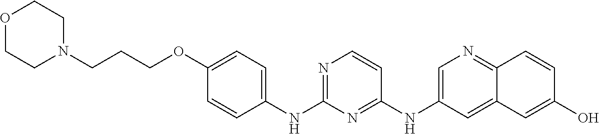 [SMILES: Oc1ccc2ncc(Nc3ccnc(Nc4ccc(OCCCN5CCOCC5)cc4)n3)cc2c1] | 3-{2-[p-(3- morpholinopropoxy) phenylamino]- 4-pyrimidinylamino}- 6-quinolinol |
| 415 | 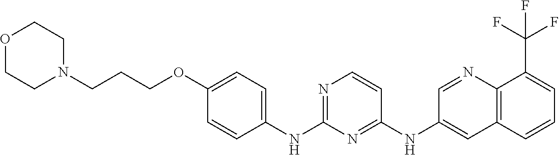 [SMILES: FC(F)(F)c1cccc2cc(Nc3ccnc(Nc4ccc(OCCCN5CCOCC5)cc4)n3)cnc12] | 2-[p-(3- morpholinopropoxy) phenylamino]-4-[8- (trifluoromethyl)-3- quinolylamino]pyrimidine |
| 416 | 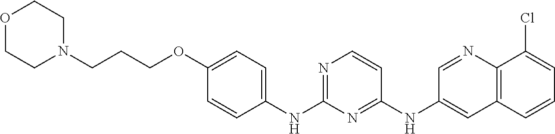 [SMILES: Clc1cccc2cc(Nc3ccnc(Nc4ccc(OCCCN5CCOCC5)cc4)n3)cnc12] | 4-(8-chloro-3- quinolylamino)-2-[p-(3- morpholinopropoxy) phenylamino]pyrimidine |
| 417 | 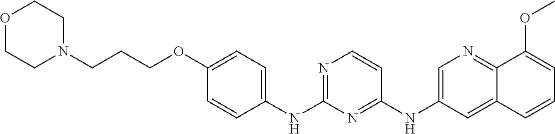 [SMILES: COc1cccc2cc(Nc3ccnc(Nc4ccc(OCCCN5CCOCC5)cc4)n3)cnc12] | 4-(8-methoxy-3- quinolylamino)-2-[p-(3- morpholinopropoxy) phenylamino]pyrimidine |
| 418 | 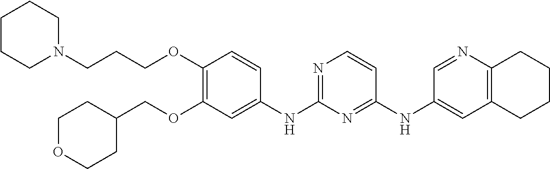 [SMILES: c1cc(Nc2cnc3c(c2)CCCC3)nc(Nc2ccc(OCCCN3CCCCC3)c(OCC3CCOCC3)c2)n1] | 2-{4-(3-piperidinopropoxy)- 3-[(tetrahydro-2H-pyran-4- yl)methoxyphenylamino}- 4-(3- quinolylamino)pyrimidine |
| 419 | 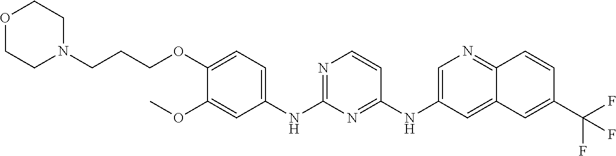 [SMILES: COc1cc(Nc2nccc(Nc3cnc4ccc(C(F)(F)F)cc4c3)n2)ccc1OCCCN1CCOCC1] | 2-[3-methoxy-4-(3- morpholinopropoxy) phenylamino]-4-[6- (trifluoromethyl)-3- quinolylamino]pyrimidine |
| 420 | 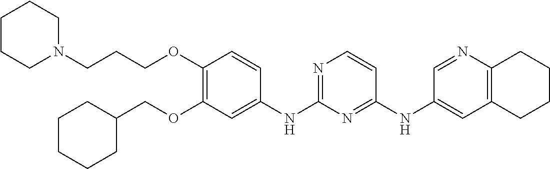 [SMILES: c1cc(Nc2cnc3c(c2)CCCC3)nc(Nc2ccc(OCCCN3CCCCC3)c(OCC3CCCCC3)c2)n1] | 2-[3-(cyclohexylmethoxy)- 4-(3- piperidinopropoxy) phenylamino]-4-(3- quinolylamino)pyrimidine |
| 421 | 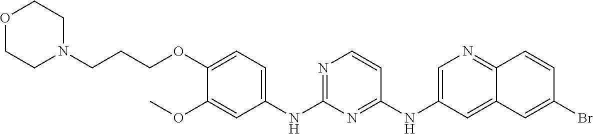 [SMILES: COc1cc(Nc2nccc(Nc3cnc4ccc(Br)cc4c3)n2)ccc1OCCCN1CCOCC1] | 4-(6-bromo-3- quinolylamino)-2-[3- methoxy-4-(3- piperidinopropoxy) phenylamino]pyrimidine |
| 422 | 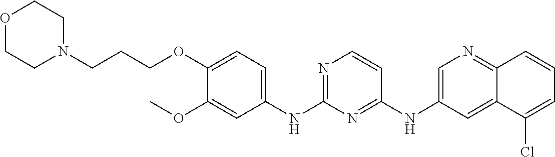 [SMILES: COc1cc(Nc2nccc(Nc3cnc4cccc(Cl)c4c3)n2)ccc1OCCCN1CCOCC1] | 4-(5-chloro-3- quinolylamino)-2-[3- methoxy-4-(3- piperidinopropoxy) phenylamino]pyrimidine |
| 423 | 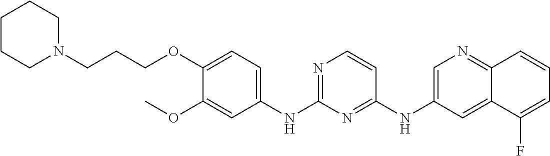 [SMILES: COc1cc(Nc2nccc(Nc3cnc4cccc(F)c4c3)n2)ccc1OCCCN1CCCCC1] | 4-(5-fluoro-3- quinolylamino)-2-[3- methoxy-4-(3- piperidinopropoxy) phenylamino]pyrimidine |
| 424 | 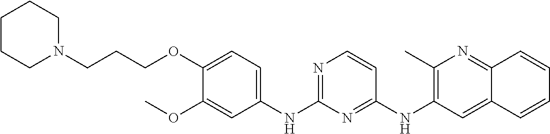 [SMILES: COc1cc(Nc2nccc(Nc3cc4ccccc4nc3C)n2)ccc1OCCCN1CCCCC1] | 2-[3-methoxy-4-(3- piperidinopropoxy) phenylamino]- 4-(2-methyl-3- quinolylamino)pyrimidine |
| 425 | 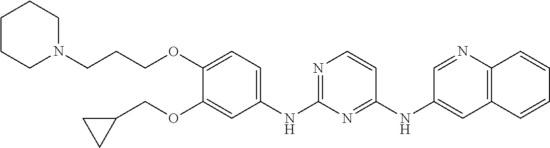 [SMILES: c1ccc2ncc(Nc3ccnc(Nc4ccc(OCCCN5CCCCC5)c(OCC5CC5)c4)n3)cc2c1] | 2-[3-(cyclopropylmethoxy)- 4-(3- piperidinopropoxy) phenylamino]-4-(3- quinolylamino)pyrimidine |
| 426 | 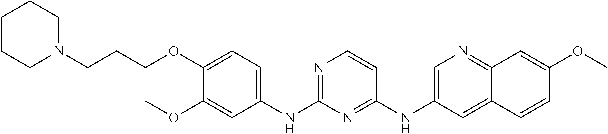 [SMILES: COc1ccc2cc(Nc3ccnc(Nc4ccc(OCCCN5CCCCC5)c(OC)c4)n3)cnc2c1] | 2-[3-methoxy-4-(3- piperidinopropoxy) phenylamino]- 4-(7-methoxy-3- quinolylamino)pyrimidine |
| 427 | 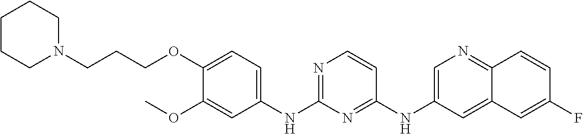 [SMILES: COc1cc(Nc2nccc(Nc3cnc4ccc(F)cc4c3)n2)ccc1OCCCN1CCCCC1] | 4-(6-fluoro-3- quinolylamino)-2-[3- methoxy-4-(3- piperidinopropoxy) phenylamino]pyrimidine |
| 428 | 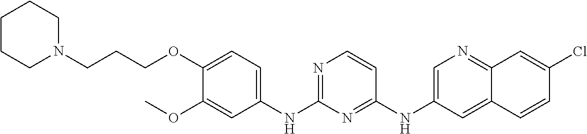 [SMILES: COc1cc(Nc2nccc(Nc3cnc4cc(Cl)ccc4c3)n2)ccc1OCCCN1CCCCC1] | 4-(7-chloro-3- quinolylamino)-2-[3- methoxy-4-(3- piperidinopropoxy) phenylamino]pyrimidine |
| 429 | 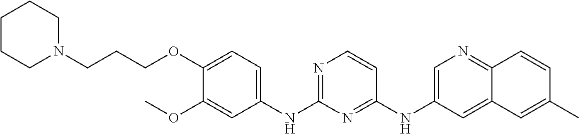 [SMILES: COc1cc(Nc2nccc(Nc3cnc4ccc(C)cc4c3)n2)ccc1OCCCN1CCCCC1] | 2-[3-methoxy-4-(3- piperidinopropoxy) phenylamino]- 4-(6-methyl-3- quinolylamino)pyrimidine |
| 430 | 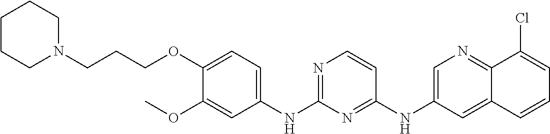 [SMILES: COc1cc(Nc2nccc(Nc3cnc4c(Cl)cccc4c3)n2)ccc1OCCCN1CCCCC1] | 4-(8-chloro-3- quinolylamino)-2-[3- methoxy-4-(3- piperidinopropoxy) phenylamino]pyrimidine |
| 431 | 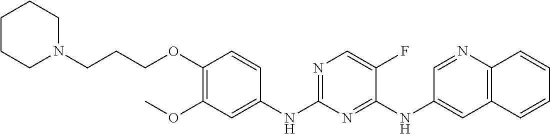 [SMILES: COc1cc(Nc2ncc(F)c(Nc3cnc4ccccc4c3)n2)ccc1OCCCN1CCCCC1] | 5-fluoro-2-[3-methoxy-4-(3- piperidinopropoxy) phenylamino]-4-(3- quinolylamino)pyrimidine |
| 432 | 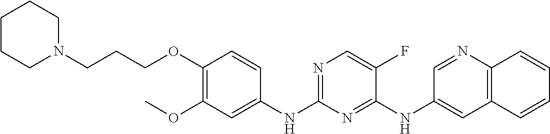 [SMILES: COc1cc(Nc2ncc(F)c(Nc3cnc4ccccc4c3)n2)ccc1OCCCN1CCCCC1] | 5-fluoro-2-[p-(3- piperidinopropoxy) phenylamino]-4-(3- quinolylamino)pyrimidine |

In some embodiments, the compound is a pharmaceutically acceptable salt of a compound described in Table 5.

TABLE 6

| Cmpd | | |
|---|---|---|
| # | Structure | Name |
| 433 | 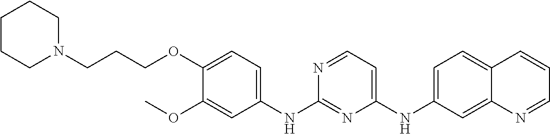 [SMILES: COc1cc(Nc2nccc(Nc3ccc4cccnc4c3)n2)ccc1OCCCN1CCCCC1] | 2-[3-methoxy-4-(3- piperidinopropoxy) phenylamino]- 4-(7-quinolylamino) pyrimidine |
| 434 | 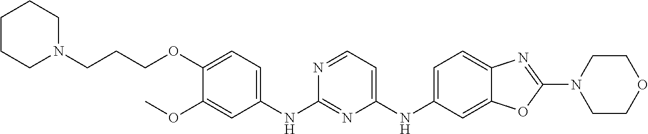 [SMILES: COc1cc(Nc2nccc(Nc3ccc4nc(N5CCOCC5)oc4c3)n2)ccc1OCCCN1CCCCC1] | 2-[3-methoxy-4-(3- piperidinopropoxy) phenylamino]- 4-(2-morpholino-1,3- benzoxazol-6- ylamino)pyrimidine |
| 435 | 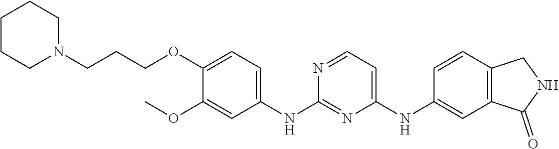 [SMILES: COc1cc(Nc2nccc(Nc3ccc4c(c3)C(=O)NC4)n2)ccc1OCCCN1CCCCC1] | 6-{2-[3-methoxy-4-(3- piperidinopropoxy) phenylamino]- 4-pyrimidinylamino}- 1-isoindolinone |
| 436 | 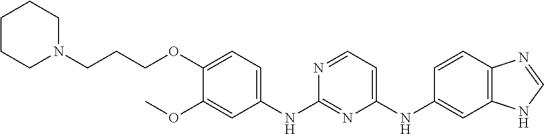 [SMILES: COc1cc(Nc2nccc(Nc3ccc4nc[nH]c4c3)n2)ccc1OCCCN1CCCCC1] | 4-(1H-1,3-benzimidazol-6- ylamino)-2-[3-methoxy-4-(3- piperidinopropoxy) phenylamino] pyrimidine |
| 437 | 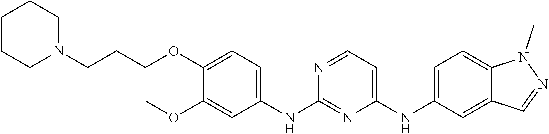 [SMILES: COc1cc(Nc2nccc(Nc3ccc4c(cnn4C)c3)n2)ccc1OCCCN1CCCCC1] | 2-[3-methoxy-4-(3- piperidinopropoxy) phenylamino]- 4-(1-methyl-1H- indazol-5- ylamino)pyrimidine |
| 438 | 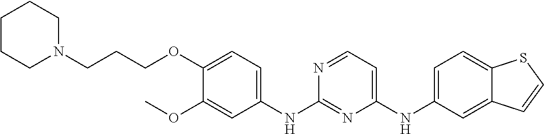 [SMILES: COc1cc(Nc2nccc(Nc3ccc4sccc4c3)n2)ccc1OCCCN1CCCCC1] | 4-(1-benzothiophen-5- ylamino)-2-[3-methoxy-4-(3- piperidinopropoxy) phenylamino] pyrimidine |
| 439 | 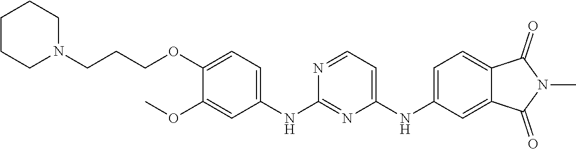 [SMILES: COc1cc(Nc2nccc(Nc3ccc4c(c3)C(=O)N(C)C4=O)n2)ccc1OCCCN1CCCCC1] | 5-{2-[3-methoxy-4-(3- piperidinopropoxy) phenylamino]- 4-pyrimidinylamino}- 2-methyl-1,3- isoindolinedione |
| 440 | 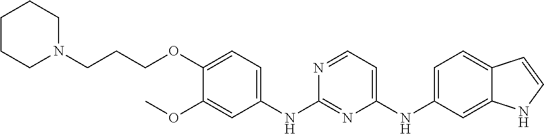 [SMILES: COc1cc(Nc2nccc(Nc3ccc4cc[nH]c4c3)n2)ccc1OCCCN1CCCCC1] | 4-(6-indolylamino)-2-[3- methoxy-4-(3- piperidinopropoxy) phenylamino] pyrimidine |
| 441 | 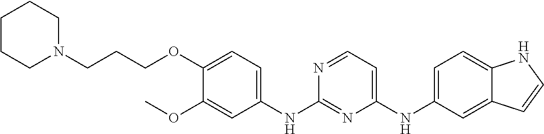 [SMILES: COc1cc(Nc2nccc(Nc3ccc4[nH]ccc4c3)n2)ccc1OCCCN1CCCCC1] | 4-(5-indolylamino)-2-[3- methoxy-4-(3- piperidinopropoxy) phenylamino] pyrimidine |
| 442 | 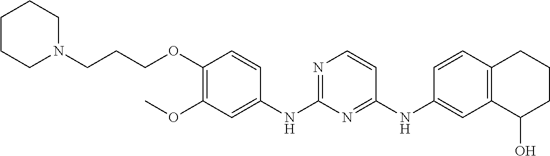 [SMILES: COc1cc(Nc2nccc(Nc3ccc4c(c3)C(O)CCC4)n2)ccc1OCCCN1CCCCC1] | 7-{2-[3-methoxy-4-(3- piperidinopropoxy) phenylamino]- 4-pyrimidinylamino}- 1,2,3,4-tetrahydro-1- naphthol |
| 443 | 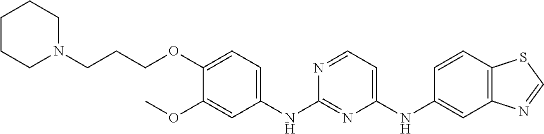 [SMILES: COc1cc(Nc2nccc(Nc3ccc4scnc4c3)n2)ccc1OCCCN1CCCCC1] | 4-(1,3-benzothiazol-5- ylamino)-2-[3-methoxy-4-(3- piperidinopropoxy) phenylamino] pyrimidine |
| 444 | 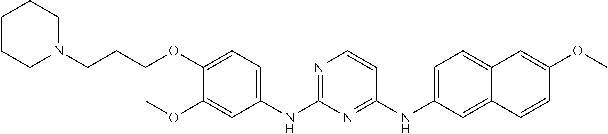 [SMILES: COc1ccc2cc(Nc3ccnc(Nc4ccc(OCCCN5CCCCC5)c(OC)c4)n3)ccc2c1] | 4-(6-methoxy-2- naphthylamino)-2-[3- methoxy-4-(3- piperidinopropoxy) phenylamino] pyrimidine |
| 445 | 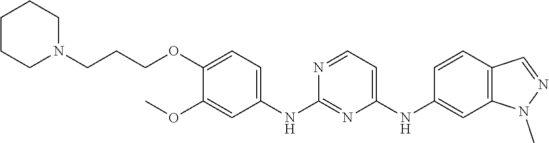 [SMILES: COc1cc(Nc2nccc(Nc3ccc4cnn(C)c4c3)n2)ccc1OCCCN1CCCCC1] | 2-[3-methoxy-4-(3- piperidinopropoxy) phenylamino]- 4-(1-methyl-1H- indazol-6- ylamino)pyrimidine |
| 446 | 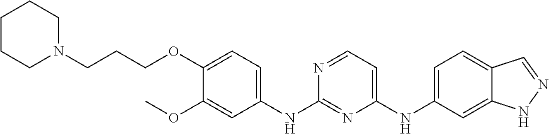 [SMILES: COc1cc(Nc2nccc(Nc3ccc4cn[nH]c4c3)n2)ccc1OCCCN1CCCCC1] | 4-(1H-indazol-6-ylamino)-2- [3-methoxy-4-(3- piperidinopropoxy) phenylamino] pyrimidine |
| 447 | 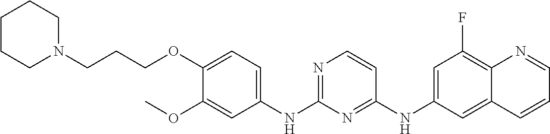 [SMILES: COc1cc(Nc2nccc(Nc3cc(F)c4ncccc4c3)n2)ccc1OCCCN1CCCCC1] | 4-(8-fluoro-6- quinolylamino)-2-[3- methoxy-4-(3- piperidinopropoxy) phenylamino] pyrimidine |
| 448 | 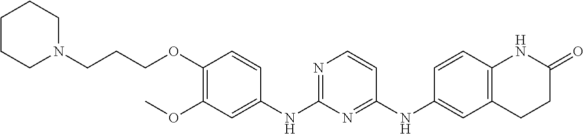 [SMILES: COc1cc(Nc2nccc(Nc3ccc4c(c3)CCC(=O)N4)n2)ccc1OCCCN1CCCCC1] | 6-{2-[3-methoxy-4-(3- piperidinopropoxy) phenylamino]-4-pyrimidinylamino}- 3,4-dihydro-2(1H)- quinolinone |
| 449 | 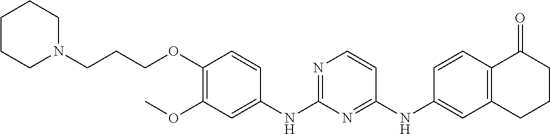 [SMILES: COc1cc(Nc2nccc(Nc3ccc4c(c3)CCCC4=O)n2)ccc1OCCCN1CCCCC1] | 6-{2-[3-methoxy-4-(3- piperidinopropoxy) phenylamino]- 4-pyrimidinylamino}- 3,4-dihydro-1(2H)- naphthalenone |
| 450 | 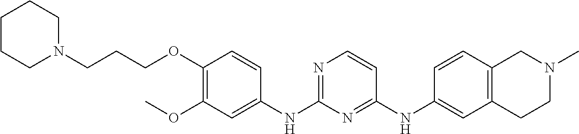 [SMILES: COc1cc(Nc2nccc(Nc3ccc4c(c3)CCN(C)C4)n2)ccc1OCCCN1CCCCC1] | 2-[3-methoxy-4-(3- piperidinopropoxy) phenylamino]- 4-(2-methyl-1,2,3,4- tetrahydro-6- isoquinolylamino) pyrimidine |
| 451 | 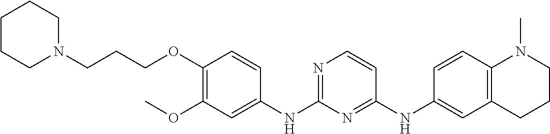 [SMILES: COc1cc(Nc2nccc(Nc3ccc4c(c3)CCCN4C)n2)ccc1OCCCN1CCCCC1] | 2-[3-methoxy-4-(3- piperidinopropoxy) phenylamino]- 4-(1-methyl-1,2,3,4- tetrahydro-6- quinolylamino) pyrimidine |
| 452 | 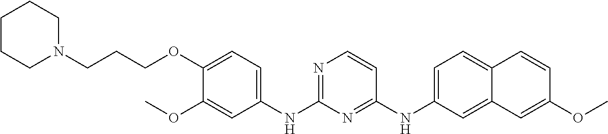 [SMILES: COc1ccc2ccc(Nc3ccnc(Nc4ccc(OCCCN5CCCCC5)c(OC)c4)n3)cc2c1] | 4-(7-methoxy-2- naphthylamino)-2-[3- methoxy-4-(3- piperidinopropoxy) phenylamino] pyrimidine |
| 453 | 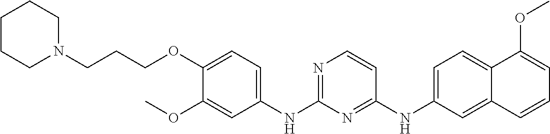 [SMILES: COc1cc(Nc2nccc(Nc3ccc4c(OC)cccc4c3)n2)ccc1OCCCN1CCCCC1] | 4-(5-methoxy-2- naphthylamino)-2-[3- methoxy-4-(3- piperidinopropoxy) phenylamino] pyrimidine |
| 454 | 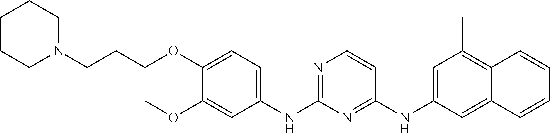 [SMILES: COc1cc(Nc2nccc(Nc3cc(C)c4ccccc4c3)n2)ccc1OCCCN1CCCCC1] | 2-[3-methoxy-4-(3- piperidinopropoxy) phenylamino]- 4-(4-methyl-2-naphthylamino) pyrimidine |
| 455 | 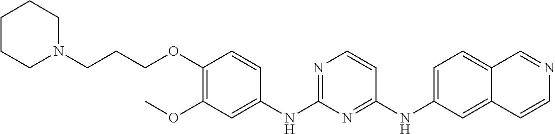 [SMILES: COc1cc(Nc2nccc(Nc3ccc4cnccc4c3)n2)ccc1OCCCN1CCCCC1] | 4-(6-isoquinolylamino)-2-[3- methoxy-4-(3- piperidinopropoxy) phenylamino] pyrimidine |
| 456 | 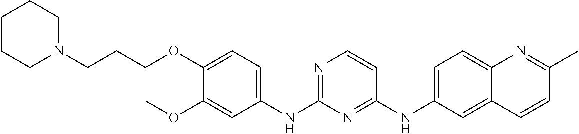 [SMILES: COc1cc(Nc2nccc(Nc3ccc4nc(C)ccc4c3)n2)ccc1OCCCN1CCCCC1] | 2-[3-methoxy-4-(3- piperidinopropoxy) phenylamino]- 4-(2-methyl-6-quinolylamino) pyrimidine |
| 457 | 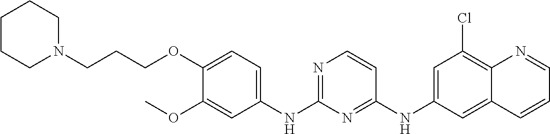 [SMILES: COc1cc(Nc2nccc(Nc3cc(Cl)c4ncccc4c3)n2)ccc1OCCCN1CCCCC1] | 4-(8-chloro-6- quinolylamino)-2-[3- methoxy-4-(3- piperidinopropoxy) phenylamino] pyrimidine |
| 458 | 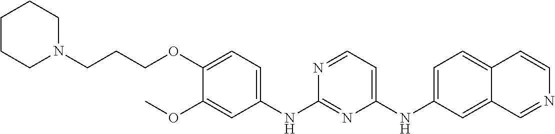 [SMILES: COc1cc(Nc2nccc(Nc3ccc4ccncc4c3)n2)ccc1OCCCN1CCCCC1] | 4-(7-isoquinolylamino)-2-[3- methoxy-4-(3- piperidinopropoxy) phenylamino] pyrimidine |

In some embodiments, the compound is a pharmaceutically acceptable salt of a compound described in Table 6.

TABLE 7

| Cmpd # | Structure | Name |
|---|---|---|
| 1 | 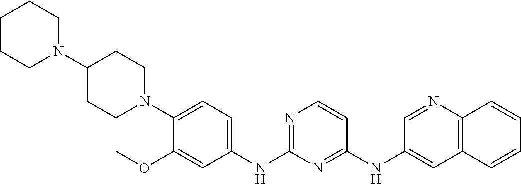 [SMILES: COc1cc(Nc2nccc(Nc3cnc4ccccc4c3)n2)ccc1N1CCC(N2CCCCC2)CC1] | 2-[4-(1,4′-bipiperidy1-1′-yl)- 3-anisidino]-4-(3-quinolylamino)pyrimidine |
| 2 | 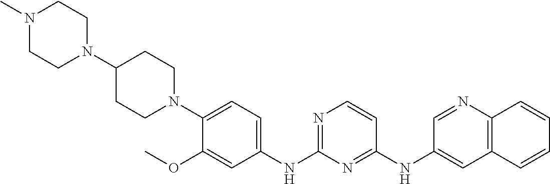 [SMILES: COc1cc(Nc2nccc(Nc3cnc4ccccc4c3)n2)ccc1N1CCC(N2CCN(C)CC2)CC1] | 2-{3-methoxy-4-[4-(4-methyl-1- piperazinyl)-1- piperidyl]phenylamino}-4-(3- quinolylamino)pyrimidine |
| 3 | 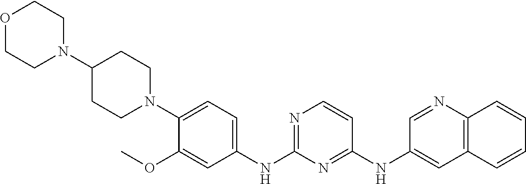 [SMILES: COc1cc(Nc2nccc(Nc3cnc4ccccc4c3)n2)ccc1N1CCC(N2CCOCC2)CC1] | 2-[3-methoxy-4-(4-morpholino-1- piperidyl)phenylamino]-4-(3- quinolylamino)pyrimidine |
| 48 | 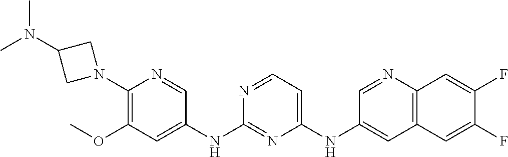 [SMILES: COc1cc(Nc2nccc(Nc3cnc4cc(F)c(F)cc4c3)n2)cnc1N1CC(N(C)C)C1] | 4-(6,7-difluoro-3-quinolylamino)- 2-{6-[3-(dimethylamino)-1-azetidinyl]- 5-methoxy-3-pyridylamino}pyrimidine |
| 49 | 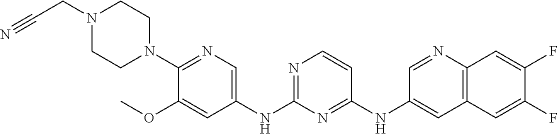 [SMILES: COc1cc(Nc2nccc(Nc3cnc4cc(F)c(F)cc4c3)n2)cnc1N1CCN(CC#N)CC1] | (4-{5-[4-(6,7-difluoro-3- quinolylamino)-2- pyrimidinylamino]- 3-methoxy-2-pyridyl-1 piperazinyl)acetonitrile |
| 50 | 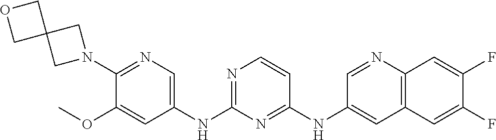 [SMILES: COc1cc(Nc2nccc(Nc3cnc4cc(F)c(F)cc4c3)n2)cnc1N1CC2(COC2)C1] | 4-(6,7-difluoro-3-quinolylamino)- 2-{5-methoxy-6-(2-oxa-6-aza-6-spiro[3.3]heptyl)-3-pyridylamino}pyrimidine |
| 51 | 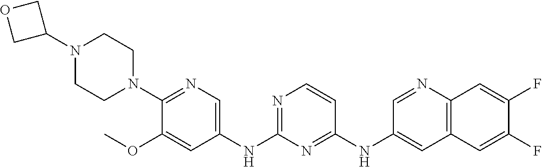 [SMILES: COc1cc(Nc2nccc(Nc3cnc4cc(F)c(F)cc4c3)n2)cnc1N1CCN(C2COC2)CC1] | 4-(6,7-difluoro-3-quinolylamino)- 2-{5-methoxy-6-[4-(3-oxetanyl)- 1-piperazinyl]-3-pyridylamino}pyrimidine |
| 52 | 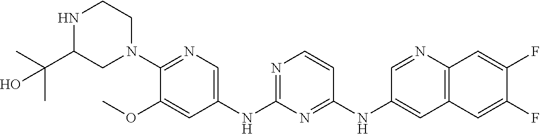 [SMILES: COc1cc(Nc2nccc(Nc3cnc4cc(F)c(F)cc4c3)n2)cnc1N1CCNC(C(C)(C)O)C1] | 2-(4-{5-[4-(6,7-difluoro-3- quinolylamino)-2- pyrimidinylamino]-3-methoxy-2- pyridyl}-2-piperazinyl)-2- propanol |
| 90 | 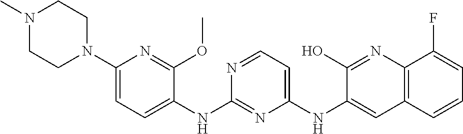 [SMILES: COc1nc(N2CCN(C)CC2)ccc1Nc1nccc(Nc2cc3cccc(F)c3nc2O)n1] | 8-fluoro-3-{2-[2-methoxy-6-(4- methyl-1-piperazinyl)-3- pyridylamino]-4-pyrimidinylamino}- 2-quinolinol |
| 91 | 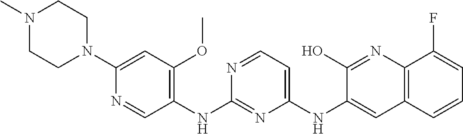 [SMILES: COc1cc(N2CCN(C)CC2)ncc1Nc1nccc(Nc2cc3cccc(F)c3nc2O)n1] | 8-fluoro-3-{2-[4-methoxy-6-(4- methyl-1-piperazinyl)-3- pyridylamino]-4-pyrimidinylamino}- 2-quinolinol |
| 94 | 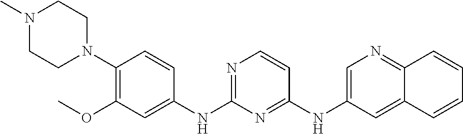 [SMILES: COc1cc(Nc2nccc(Nc3cnc4ccccc4c3)n2)ccc1N1CCN(C)CC1] | 2-[3-methoxy-4-(4-methyl-1- piperazinyl)phenylamino]-4- (3-quinolylamino)pyrimidine |
| 95 | 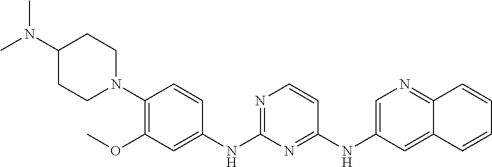 [SMILES: COc1cc(Nc2nccc(Nc3cnc4ccccc4c3)n2)ccc1N1CCC(N(C)C)CC1] | 2-{4-[4-(dimethylamino)-1- piperidyl]-3- anisidino}-4-(3- quinolylamino)pyrimidine |
| 126 | 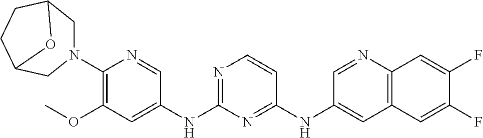 [SMILES: COc1cc(Nc2nccc(Nc3cnc4cc(F)c(F)cc4c3)n2)cnc1N1CC2CCC(C1)O2] | 4-(6,7-difluoro-3-quinolylamino)- 2-{5-methoxy-6- (8-oxa-3- azabicyclo[3.2.1]oct-3-y1)- 3-pyridylamino}pyrimidine |
| 127 | 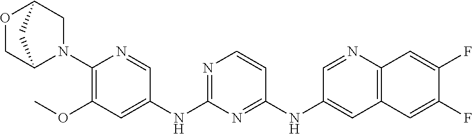 [SMILES: COc1cc(Nc2nccc(Nc3cnc4cc(F)c(F)cc4c3)n2)cnc1N1C[C@H]2C[C@@H]1CO2] | 2-(6-{(1R,4R)-2-oxa-5- azabicyclo[2.2.1]hept-5-yl}-5- methoxy-3-pyridylamino)-4-(6,7- difluoro-3- quinolylamino)pyrimidine |
| 155 | 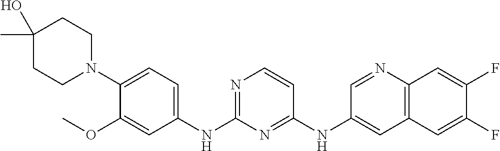 [SMILES: COc1cc(Nc2nccc(Nc3cnc4cc(F)c(F)cc4c3)n2)ccc1N1CCC(C)(O)CC1] | 1-{4-[4-(6,7-difluoro-3- quinolylamino)-2- pyrimidinylamino]-2- methoxyphenyl}-4-methyl-4- piperidinol |
| 156 | 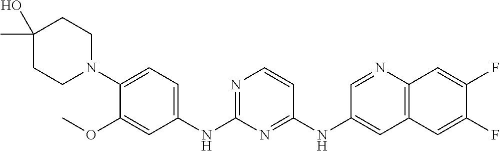 [SMILES: COc1cc(Nc2nccc(Nc3cnc4cc(F)c(F)cc4c3)n2)ccc1N1CCC(C)(O)CC1] | 1-{5-[4-(6,7-difluoro-3- quinolylamino)-2- pyrimidinylamino]-3-methoxy-2-pyridyl}-4-methyl-4-piperidinol |
| 157 | 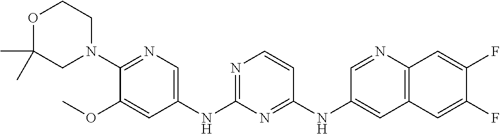 [SMILES: COc1cc(Nc2nccc(Nc3cnc4cc(F)c(F)cc4c3)n2)cnc1N1CCOC(C)(C)C1] | 4-(6,7-difluoro-3-quinolylamino)- 2-[6-(2,2-dimethyl-4- morpholinyl)-5-methoxy-3- pyridylamino]pyrimidine |
| 158 | 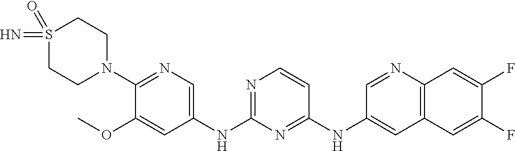 [SMILES: COc1cc(Nc2nccc(Nc3cnc4cc(F)c(F)cc4c3)n2)cnc1N1CCS(=N)(=O)CC1] | 4-{5-[4-(6,7-difluoro-3- quinolylamino)-2- pyrimidinylamino]-3-methoxy-2- pyridyl}-1-imino-126,4-thiazinan- 1-one |
| 161 | 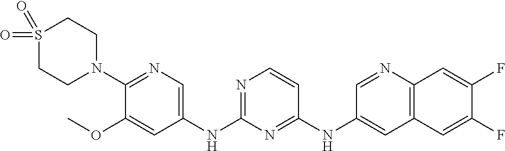 [SMILES: COc1cc(Nc2nccc(Nc3cnc4cc(F)c(F)cc4c3)n2)cnc1N1CCS(=O)(=O)CC1] | 4-{5-[4-(6,7-difluoro-3- quinolylamino)-2- pyrimidinylamino]-3-methoxy-2- pyridy}-1λ⁶,4-thiazinane-1,1- dione |
| 176 | 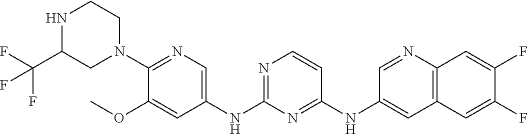 [SMILES: COc1cc(Nc2nccc(Nc3cnc4cc(F)c(F)cc4c3)n2)cnc1N1CCNC(C(F)(F)F)C1] | 4-(6,7-difluoro-3-quinolylamino)- 2-{5-methoxy-6- [3-(trifluoromethyl)- 1-piperazinyl]-3-pyridylamino}pyrimidine |
| 207 | 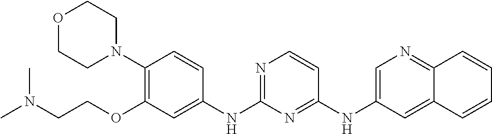 [SMILES: CN(C)CCOc1cc(Nc2nccc(Nc3cnc4ccccc4c3)n2)ccc1N1CCOCC1] | 2-{3-[2-(dimethylamino)ethoxy]- 4-morpholinophenylamino}-4-(3- quinolylamino)pyrimidine |
| 459 | 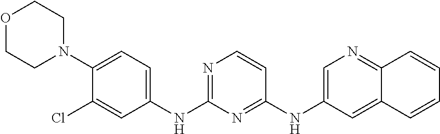 [SMILES: Clc1cc(Nc2nccc(Nc3cnc4ccccc4c3)n2)ccc1N1CCOCC1] | 2-(3-chloro-4- morpholinophenylamino)-4-(3- quinolylamino)pyrimidine |
| 460 | 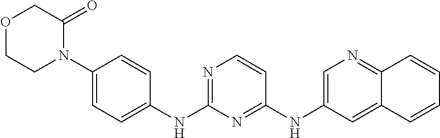 [SMILES: O=C1COCCN1c1ccc(Nc2nccc(Nc3cnc4ccccc4c3)n2)cc1] | 4-{p-[4-(3-quinolylamino)-2- pyrimidinylamino]phenyl}-3-morpholinone |
| 461 | 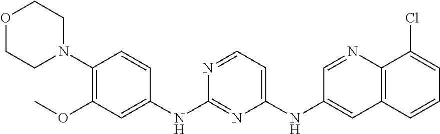 [SMILES: COc1cc(Nc2nccc(Nc3cnc4c(Cl)cccc4c3)n2)ccc1N1CCOCC1] | 4-(8-chloro-3-quinolylamino)-2- (3-methoxy-4- morpholinophenylamino) pyrimidine |
| 462 | 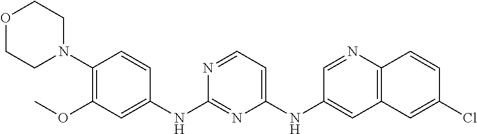 [SMILES: COc1cc(Nc2nccc(Nc3cnc4ccc(Cl)cc4c3)n2)ccc1N1CCOCC1] | 4-(6-chloro-3-quinolylamino)-2- (3-methoxy-4- morpholinophenylamino) pyrimidine |
| 463 | 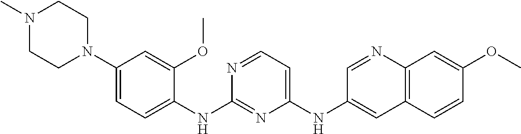 | 2-[2-methoxy-4-(4-methyl-1- piperazinyl)phenylamino]-4-(7- methoxy-3- quinolylamino)pyrimidine |
| 464 | 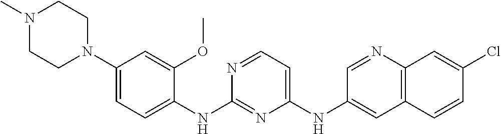 [SMILES: COc1cc(N2CCN(C)CC2)ccc1Nc1nccc(Nc2cnc3cc(Cl)ccc3c2)n1] | 4-(7-chloro-3-quinolylamino)-2- [2-methoxy-4- (4-methyl-1- piperazinyl)phenylamino] pyrimidine |
| 465 | 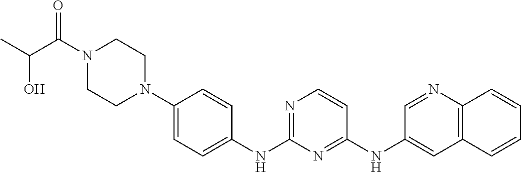 [SMILES: CC(O)C(=O)N1CCN(c2ccc(Nc3nccc(Nc4cnc5ccccc5c4)n3)cc2)CC1] | 2-hydroxy-1-(4-{p-[4-(3- quinolylamino)-2- pyrimidinylamino]phenyl}- 1-piperazinyl)-1-propanone |
| 466 | 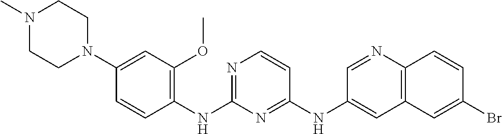 [SMILES: COc1cc(N2CCN(C)CC2)ccc1Nc1nccc(Nc2cnc3ccc(Br)cc3c2)n1] | 4-(6-bromo-3-quinolylamino)-2- [2-methoxy-4-(4-methyl-1- piperazinyl)phenylamino] pyrimidine |
| 467 | 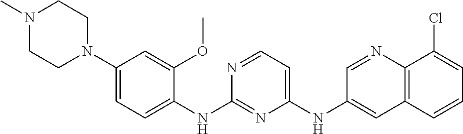 [SMILES: COc1cc(N2CCN(C)CC2)ccc1Nc1nccc(Nc2cnc3c(Cl)cccc3c2)n1] | 4-(8-chloro-3-quinolylamino)-2- [2-methoxy-4-(4-methyl-1- piperazinyl)phenylamino] pyrimidine |
| 468 | 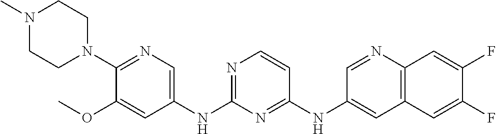 [SMILES: COc1cc(Nc2nccc(Nc3cnc4cc(F)c(F)cc4c3)n2)cnc1N1CCN(C)CC1] | 4-(6,7-difluoro-3-quinolylamino)- 2-[5-methoxy-6-(4-methyl-1- piperazinyl)-3- pyridylamino]pyrimidine |
| 469 | 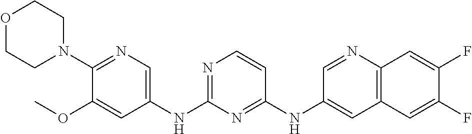 [SMILES: COc1cc(Nc2nccc(Nc3cnc4cc(F)c(F)cc4c3)n2)cnc1N1CCOCC1] | 4-(6,7-difluoro-3-quinolylamino)- 2-(5-methoxy-6-morpholino-3- pyridylamino)pyrimidine |
| 470 | 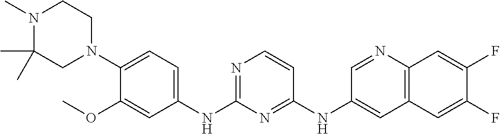 [SMILES: COc1cc(Nc2nccc(Nc3cnc4cc(F)c(F)cc4c3)n2)ccc1N1CCN(C)C(C)(C)C1] | 4-(6,7-difluoro-3-quinolylamino)- 2-[4-(3,3-dimethyl-4-methyl-1- piperazinyl)-3- anisidino]pyrimidine |
| 470 | 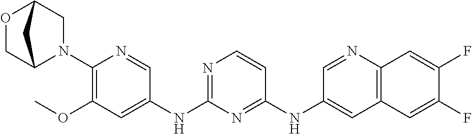 [SMILES: COc1cc(Nc2nccc(Nc3cnc4cc(F)c(F)cc4c3)n2)cnc1N1C[C@@H]2C[C@H]1CO2] | 2-(6-{(1S,4S)-2-oxa-5- azabicyclo[2.2.1]hept-5-yl}-5- methoxy-3-pyridylamino)-4-(6,7- difluoro-3- quinolylamino)pyrimidine |
| 471 | 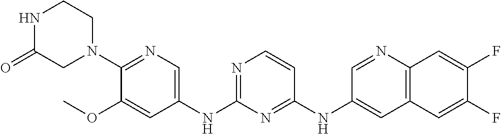 [SMILES: COc1cc(Nc2nccc(Nc3cnc4cc(F)c(F)cc4c3)n2)cnc1N1CCNC(=O)C1] | 4-{5-[4-(6,7-difluoro-3- quinolylamino)-2- pyrimidinylamino]- 3-methoxy-2-pyridyl}- 2-piperazinone |
| 472 | 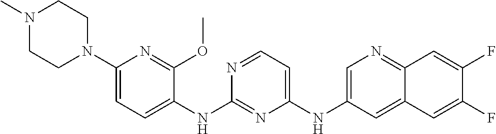 [SMILES: COc1nc(N2CCN(C)CC2)ccc1Nc1nccc(Nc2cnc3cc(F)c(F)cc3c2)n1] | 4-(6,7-difluoro-3-quinolylamino)- 2-[2-methoxy-6-(4-methyl-1- piperazinyl)-3- pyridylamino]pyrimidine |
| 473 | 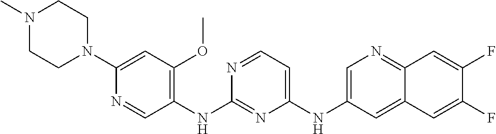 [SMILES: COc1cc(N2CCN(C)CC2)ncc1Nc1nccc(Nc2cnc3cc(F)c(F)cc3c2)n1] | 4-(6,7-difluoro-3-quinolylamino)- 2-[4-methoxy-6-(4-methy1-1- piperazinyl)-3- pyridylamino]pyrimidine |
| 474 | 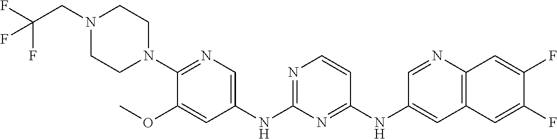 [SMILES: COc1cc(Nc2nccc(Nc3cnc4cc(F)c(F)cc4c3)n2)cnc1N1CCN(CC(F)(F)F)CC1] | 4-(6,7-difluoro-3-quinolylamino)- 2-{5-methoxy-6- [4-(2,2,2-trifluoroethyl)- 1-piperazinyl]-3-pyridylamino}pyrimidine |
| 475 | 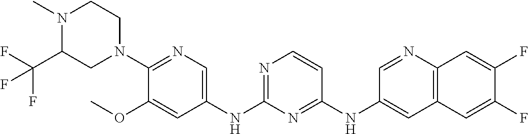 [SMILES: COc1cc(Nc2nccc(Nc3cnc4cc(F)c(F)cc4c3)n2)cnc1N1CCN(C)C(C(F)(F)F)C1] | 4-(6,7-difluoro-3- quinolylamino)- 2-{5-methoxy-6- [4-methyl-3- (trifluoromethyl)-1- piperazinyl]-3- pyridylamino}pyrimidine |
| 476 | 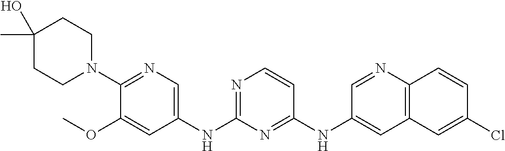 [SMILES: COc1cc(Nc2nccc(Nc3cnc4ccc(Cl)cc4c3)n2)cnc1N1CCC(C)(O)CC1] | 1-{5-[4-(6-chloro-3- quinolylamino)-2- pyrimidinylamino]-3-methoxy-2-pyridyl}-4-methyl-4-piperidinol |
| 477 | 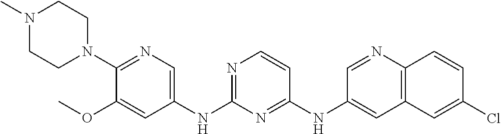 [SMILES: COc1cc(Nc2nccc(Nc3cnc4ccc(Cl)cc4c3)n2)cnc1N1CCN(C)CC1] | 4-(6-chloro-3-quinolylamino)-2- [5-methoxy-6-(4-methyl-1- piperazinyl)-3- pyridylamino]pyrimidine |
| 478 | 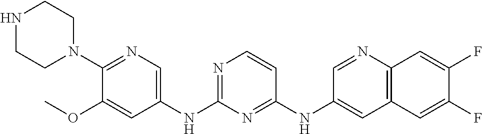 [SMILES: COc1cc(Nc2nccc(Nc3cnc4cc(F)c(F)cc4c3)n2)cnc1N1CCNCC1] | 4-(6,7-difluoro-3-quinolylamino)- 2-[5-methoxy-6- (1-piperazinyl)- 3-pyridylamino]pyrimidine |
| 479 | 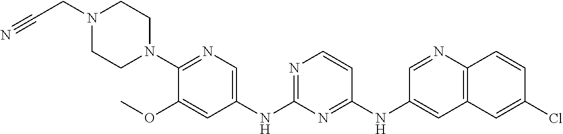 [SMILES: COc1cc(Nc2nccc(Nc3cnc4ccc(Cl)cc4c3)n2)cnc1N1CCN(CC#N)CC1] | (4-{5-[4-(6-chloro-3- quinolylamino)-2- pyrimidinylamino]-3-methoxy-2-pyridyl}-1-piperazinyl)acetonitrile |
| 480 | 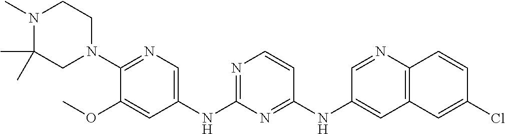 [SMILES: COc1cc(Nc2nccc(Nc3cnc4ccc(Cl)cc4c3)n2)cnc1N1CCN(C)C(C)(C)C1] | 4-(6-chloro-3-quinolylamino)-2- [6-(3,3-dimethyl-4- methyl-1-piperazinyl)-5- methoxy-3- pyridylamino] pyrimidine |
| 481 | 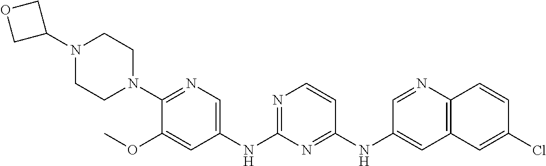 [SMILES: COc1cc(Nc2nccc(Nc3cnc4ccc(Cl)cc4c3)n2)cnc1N1CCN(C2COC2)CC1] | 4-(6-chloro-3-quinolylamino)-2- {5-methoxy-6- [4-(3-oxetanyl)- 1-piperazinyl]-3- pyridylamino}pyrimidine |
| 482 | 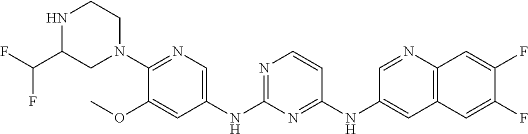 [SMILES: COc1cc(Nc2nccc(Nc3cnc4cc(F)c(F)cc4c3)n2)cnc1N1CCNC(C(F)F)C1] | 2-{6-[3-(difluoromethyl)-1- piperazinyl]-5-methoxy-3- pyridylamino}-4-(6,7-difluoro-3- quinolylamino)pyrimidine |
| 483 | 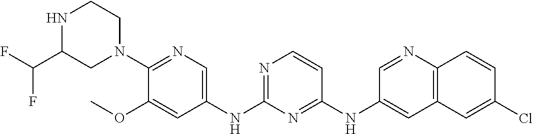 [SMILES: COc1cc(Nc2nccc(Nc3cnc4ccc(Cl)cc4c3)n2)cnc1N1CCNC(C(F)F)C1] | 4-(6-chloro-3-quinolylamino)-2- {6-[3-(difluoromethyl)- 1-piperazinyl]- 5-methoxy-3- pyridylamino}pyrimidine |
| 484 | 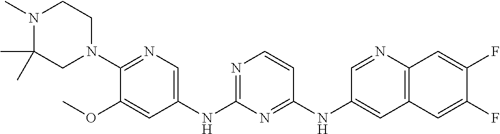 [SMILES: COc1cc(Nc2nccc(Nc3cnc4cc(F)c(F)cc4c3)n2)cnc1N1CCN(C)C(C)(C)C1] | 4-(6,7-difluoro-3-quinolylamino)- 2-[6-(3,3-dimethyl-4-methyl-1- piperazinyl)-5-methoxy-3- pyridylamino]pyrimidine |
| 485 | 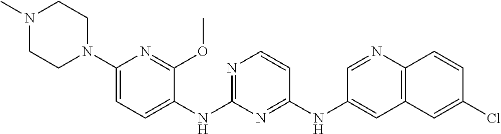 [SMILES: COc1nc(N2CCN(C)CC2)ccc1Nc1nccc(Nc2cnc3ccc(Cl)cc3c2)n1] | 4-(6-chloro-3-quinolylamino)-2- [2-methoxy-6-(4-methyl-1- piperazinyl)-3- pyridylamino]pyrimidine |
| 486 | 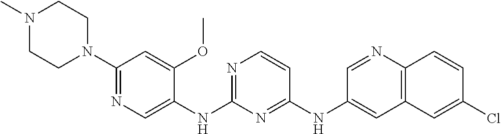 [SMILES: COc1cc(N2CCN(C)CC2)ncc1Nc1nccc(Nc2cnc3ccc(Cl)cc3c2)n1] | 4-(6-chloro-3-quinolylamino)-2- [4-methoxy-6-(4-methyl-1- piperazinyl)-3- pyridylamino]pyrimidine |
| 487 | 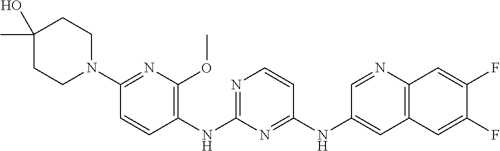 [SMILES: COc1nc(N2CCC(C)(O)CC2)ccc1Nc1nccc(Nc2cnc3cc(F)c(F)cc3c2)n1] | 1-{5-[4-(6,7-difluoro-3- quinolylamino)-2- pyrimidinylamino]-6-methoxy-2-pyridyl}-4-methyl-4-piperidinol |
| 488 | 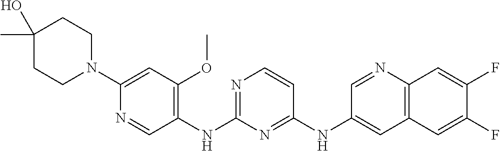 [SMILES: COc1cc(N2CCC(C)(O)CC2)ncc1Nc1nccc(Nc2cnc3cc(F)c(F)cc3c2)n1] | 1-{5-[4-(6,7-difluoro-3- quinolylamino)-2- pyrimidinylamino]-4-methoxy-2-pyridyl}-4-methyl-4-piperidinol |
| 489 | 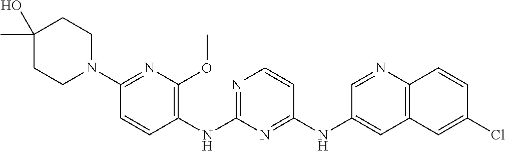 [SMILES: COc1nc(N2CCC(C)(O)CC2)ccc1Nc1nccc(Nc2cnc3ccc(Cl)cc3c2)n1] | 1-{5-[4-(6-chloro-3- quinolylamino)-2- pyrimidinylamino]-6-methoxy-2-pyridyl}-4-methyl-4-piperidinol |
| 490 | 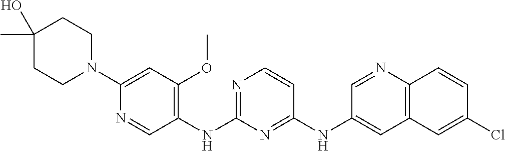 [SMILES: COc1cc(N2CCC(C)(O)CC2)ncc1Nc1nccc(Nc2cnc3ccc(Cl)cc3c2)n1] | 1-{5-[4-(6-chloro-3- quinolylamino)-2- pyrimidinylamino]-4-methoxy-2-pyridyl}-4-methyl-4-piperidinol |
| 491 | 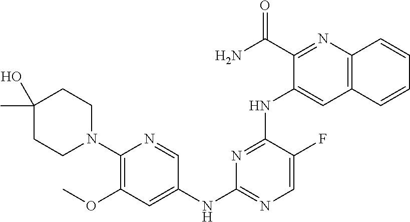 [SMILES: COc1cc(Nc2ncc(F)c(Nc3cc4ccccc4nc3C(N)=O)n2)cnc1N1CCC(C)(O)CC1] | 3-{5-fluoro-2-[6-(4-hydroxy-4- methyl-1-piperidyl)-5-methoxy-3- pyridylamino]-4- pyrimidinylamino}-2- quinolinecarboxamide |
| 492 | 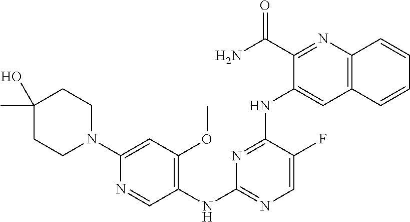 [SMILES: COc1cc(N2CCC(C)(O)CC2)ncc1Nc1ncc(F)c(Nc2cc3ccccc3nc2C(N)=O)n1] | 3-{5-fluoro-2-[6-(4-hydroxy-4- methyl-1-piperidyl)-4-methoxy-3- pyridylamino]-4- pyrimidinylamino}-2- quinolinecarboxamide |
| 493 | 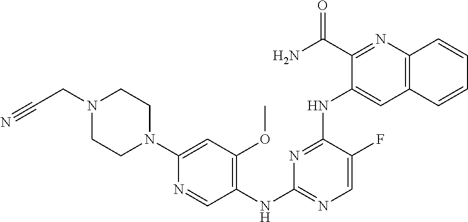 [SMILES: COc1cc(N2CCN(CC#N)CC2)ncc1Nc1ncc(F)c(Nc2cc3ccccc3nc2C(N)=O)n1] | 3-(2-{6-[4-(cyanomethyl)-1- piperazinyl]-4-methoxy-3- pyridylamino}-5-fluoro-4- pyrimidinylamino)-2- quinolinecarboxamide |
| 494 | 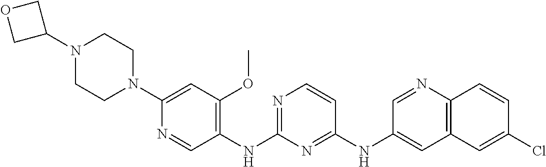 [SMILES: COc1cc(N2CCN(C3COC3)CC2)ncc1Nc1nccc(Nc2cnc3ccc(Cl)cc3c2)n1] | 4-(6-chloro-3- quinolylamino)-2- {4-methoxy-6- [4-(3-oxetanyl)- 1-piperazinyl]-3- pyridylamino}pyrimidine |
| 495 | 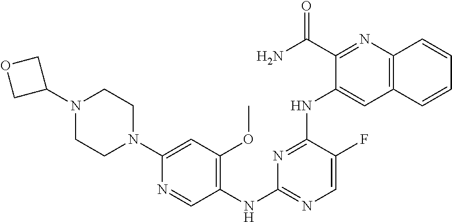 [SMILES: COc1cc(N2CCN(C3COC3)CC2)ncc1Nc1ncc(F)c(Nc2cc3ccccc3nc2C(N)=O)n1] | 3-(5-fluoro-2-{4-methoxy-6-[4-(3- oxetanyl)-1-piperazinyl]-3- pyridylamino}-4- pyrimidinylamino)-2- quinolinecarboxamide |
| 496 | 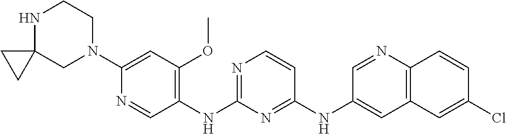 [SMILES: COc1cc(N2CCNC3(CC3)C2)ncc1Nc1nccc(Nc2cnc3ccc(Cl)cc3c2)n1] | 4-(6-chloro-3-quinolylamino)-2- {6-(4,7-diaza-7-spiro[2.5]octyl)-4- methoxy-3- pyridylamino}pyrimidine |

In some embodiments, the compound is a pharmaceutically acceptable salt of a compound described in Table 7.

TABLE 8

| Cmpd # | Structure | Name |
|---|---|---|
| 92 | 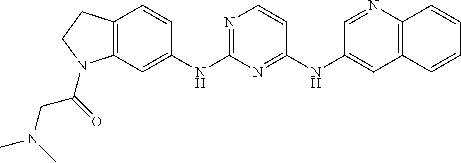 [SMILES: CN(C)CC(=O)N1CCc2ccc(Nc3nccc(Nc4cnc5ccccc5c4)n3)cc21] | 2-(dimethylamino)-1-{6-[4-(3- quinolylamino)-2- pyrimidinylamino]-1-indolinyl}-1-ethanone |
| 93 | 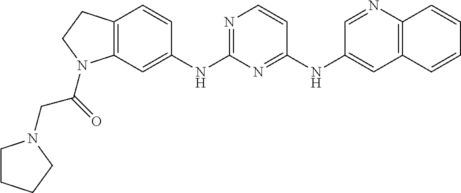 [SMILES: O=C(CN1CCCC1)N1CCc2ccc(Nc3nccc(Nc4cnc5ccccc5c4)n3)cc21] | 2-(1-pyrrolidinyl)-1-{6-[4-(3- quinolylamino)-2-pyrimidinylamino]- 1-indolinyl}-1-ethanone |
| 129 | 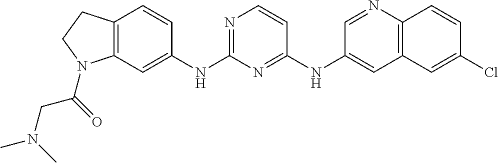 [SMILES: CN(C)CC(=O)N1CCc2ccc(Nc3nccc(Nc4cnc5ccc(Cl)cc5c4)n3)cc21] | 1-{6-[4-(6-chloro-3-quinolylamino)- 2-pyrimidinylamino]-1- indolinyl}-2- (dimethylamino)-1- ethanone |
| 130 | 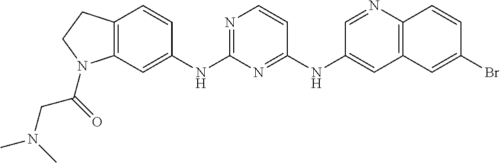 [SMILES: CN(C)CC(=O)N1CCc2ccc(Nc3nccc(Nc4cnc5ccc(Br)cc5c4)n3)cc21] | 1-{6-[4-(6-bromo-3- quinolylamino)- 2-pyrimidinylamino]- 1-indolinyl}-2- (dimethylamino)-1- ethanone |
| 135 | 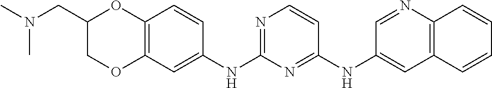 [SMILES: CN(C)CC1COc2cc(Nc3nccc(Nc4cnc5ccccc5c4)n3)ccc2O1] | 2-{2-[(dimethylamino)methyl]-2,3- dihydro-1,4-benzodioxin-6- ylamino}- 4-(3-quinolylamino)pyrimidine |
| 136 | 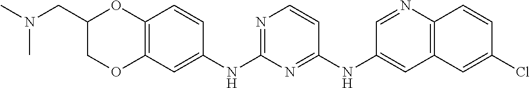 [SMILES: CN(C)CC1COc2cc(Nc3nccc(Nc4cnc5ccc(Cl)cc5c4)n3)ccc2O1] | 4-(6-chloro-3-quinolylamino)-2-{2- [(dimethylamino)methyl]-2,3- dihydro-1,4-benzodioxin-6- ylamino}pyrimidine |
| 137 | 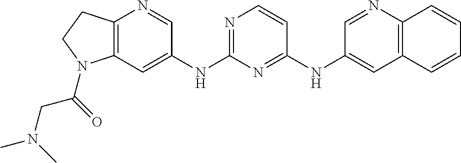 [SMILES: CN(C)CC(=O)N1CCc2ncc(Nc3nccc(Nc4cnc5ccccc5c4)n3)cc21] | 2-(dimethylamino)-1-{6-[4-(3- quinolylamino)-2- pyrimidinylamino]-1,4- diaza-1-indanyl}- 1-ethanone |
| 138 | 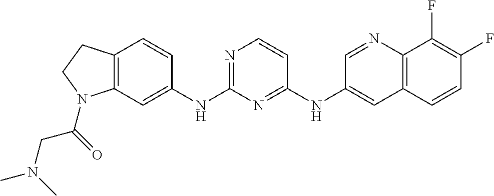 [SMILES: CN(C)CC(=O)N1CCc2ccc(Nc3nccc(Nc4cnc5c(F)c(F)ccc5c4)n3)cc21] | 1-{6-[4-(7,8-difluoro-3- quinolylamino)-2- pyrimidinylamino]- 1-indolinyl}-2-(dimethylamino)-1- ethanone |
| 139 | 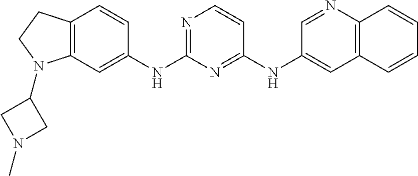 [SMILES: CN1CC(N2CCc3ccc(Nc4nccc(Nc5cnc6ccccc6c5)n4)cc32)C1] | 2-[1-(1-methyl-3-azetidinyl)-6- indolinylamino]-4-(3- quinolylamino)pyrimidine |
| 140 | 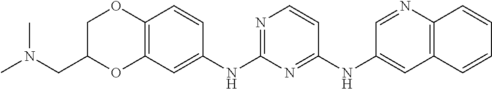 [SMILES: CN(C)CC1COc2ccc(Nc3nccc(Nc4cnc5ccccc5c4)n3)cc2O1] | 2-{3-[(dimethylamino)methyl]-2,3- dihydro-1,4-benzodioxin-6- ylamino}- 4-(3- quinolylamino)pyrimidine |
| 141 | 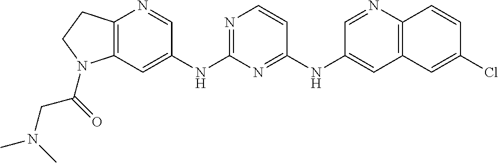 [SMILES: CN(C)CC(=O)N1CCc2ncc(Nc3nccc(Nc4cnc5ccc(Cl)cc5c4)n3)cc21] | 1-{6-[4-(6-chloro-3-quinolylamino)- 2-pyrimidinylamino]-1,4-diaza-1- indanyl}-2-(dimethylamino)-1- ethanone |
| 142 | 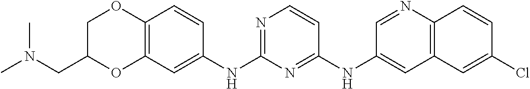 [SMILES: CN(C)CC1COc2ccc(Nc3nccc(Nc4cnc5ccc(Cl)cc5c4)n3)cc2O1] | 4-(6-chloro-3-quinolylamino)-2-{3- [(dimethylamino)methyl]-2,3- dihydro-1,4-benzodioxin-6- ylamino}pyrimidine |
| 143 | 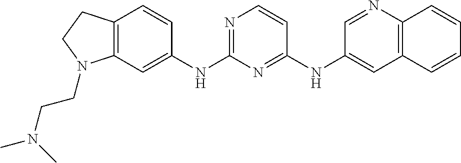 [SMILES: CN(C)CCN1CCc2ccc(Nc3nccc(Nc4cnc5ccccc5c4)n3)cc21] | 2-{1-[2-(dimethylamino)ethyl]-6- indolinylamino}-4-(3- quinolylamino)pyrimidine |
| 144 | 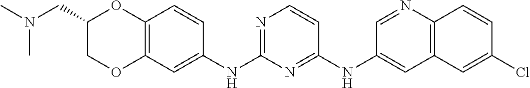 [SMILES: CN(C)C[C@H]1COc2cc(Nc3nccc(Nc4cnc5ccc(Cl)cc5c4)n3)ccc2O1] | 2-{(S)-2-[(dimethylamino)methyl]- 2,3-dihydro-1,4-benzodioxin-6- ylamino}-4-(6-chloro-3- quinolylamino)pyrimidine |
| 145 | 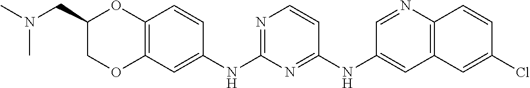 [SMILES: CN(C)C[C@@H]1COc2cc(Nc3nccc(Nc4cnc5ccc(Cl)cc5c4)n3)ccc2O1] | 2-{(R)-2-[(dimethylamino)methyl]- 2,3-dihydro-1,4-benzodioxin-6- ylamino}-4-(6-chloro-3- quinolylamino)pyrimidine |
| 146 | 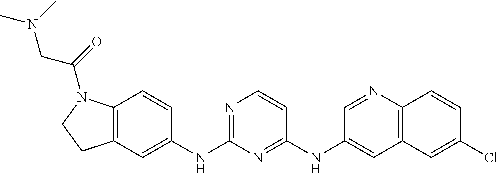 [SMILES: CN(C)CC(=O)N1CCc2cc(Nc3nccc(Nc4cnc5ccc(Cl)cc5c4)n3)ccc21] | 1-{5-[4-(6-chloro-3- quinolylamino)- 2-pyrimidinylamino]- 1-indolinyl}-2- (dimethylamino)- 1-ethanone |
| 147 | 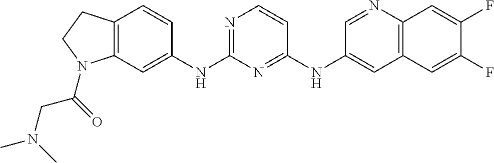 [SMILES: CN(C)CC(=O)N1CCc2ccc(Nc3nccc(Nc4cnc5cc(F)c(F)cc5c4)n3)cc21] | 1-{6-[4-(6,7-difluoro-3- quinolylamino)-2- pyrimidinylamino]- 1-indolinyl}-2-(dimethylamino)-1- ethanone |
| 148 | 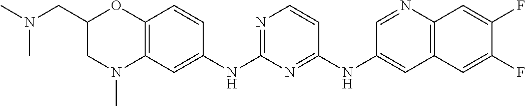 [SMILES: CN(C)CC1CN(C)c2cc(Nc3nccc(Nc4cnc5cc(F)c(F)cc5c4)n3)ccc2O1] | 4-(6,7-difluoro-3-quinolylamino)-2- {2-[(dimethylamino)methyl]-4- methyl-3,4-dihydro-2H-1,4- benzoxazin-6-ylamino}pyrimidine |
| 149 | 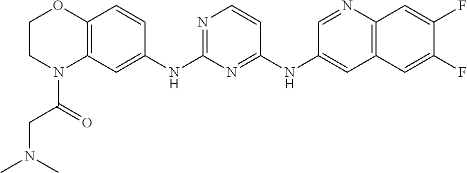 [SMILES: CN(C)CC(=O)N1CCOc2ccc(Nc3nccc(Nc4cnc5cc(F)c(F)cc5c4)n3)cc21] | 1-{6-[4-(6,7-difluoro-3- quinolylamino)-2-pyrimidinylamino]- 3,4-dihydro-2H-1,4-benzoxazin-4- yl}-2-(dimethylamino)-1-ethanone |
| 150 | 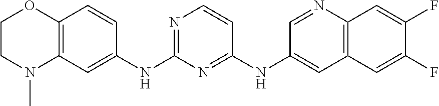 [SMILES: CN1CCOc2ccc(Nc3nccc(Nc4cnc5cc(F)c(F)cc5c4)n3)cc21] | 4-(6,7-difluoro-3-quinolylamino)-2- (4-methyl-3,4-dihydro-2H-1,4- benzoxazin-6-ylamino)pyrimidine |
| 152 | 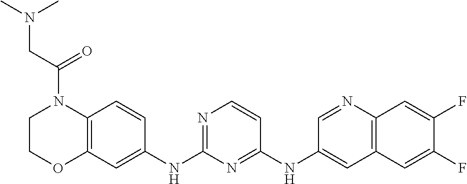 [SMILES: CN(C)CC(=O)N1CCOc2cc(Nc3nccc(Nc4cnc5cc(F)c(F)cc5c4)n3)ccc21] | 1-{7-[4-(6,7-difluoro-3- quinolylamino)-2-pyrimidinylamino]- 3,4-dihydro-2H-1,4-benzoxazin-4- yl}-2-(dimethylamino)-1-ethanone |
| 169 | 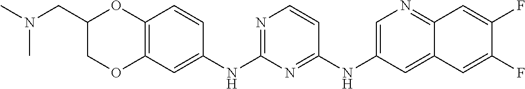 [SMILES: CN(C)CC1COc2cc(Nc3nccc(Nc4cnc5cc(F)c(F)cc5c4)n3)ccc2O1] | 4-(6,7-difluoro-3-quinolylamino)-2- {2-[(dimethylamino)methyl]-2,3- dihydro-1,4-benzodioxin-6- ylamino}pyrimidine |
| 170 | 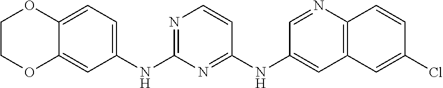 [SMILES: Clc1ccc2ncc(Nc3ccnc(Nc4ccc5c(c4)OCCO5)n3)cc2c1] | 4-(6-chloro-3-quinolylamino)-2-(2,3- dihydro-1,4-benzodioxin-6- ylamino)pyrimidine |
| 171 | 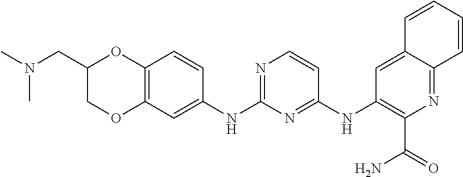 [SMILES: CN(C)CC1COc2cc(Nc3nccc(Nc4cc5ccccc5nc4C(N)=O)n3)ccc2O1] | 3-(2-{2-[(dimethylamino)methyl]- 2,3-dihydro-1,4-benzodioxin-6- ylamino}-4-pyrimidinylamino)-2- quinolinecarboxamide |
| 177 | 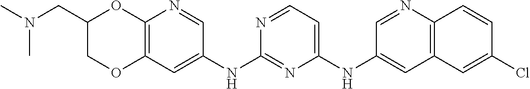 [SMILES: CN(C)CC1COc2cc(Nc3nccc(Nc4cnc5ccc(Cl)cc5c4)n3)cnc2O1] | 4-(6-chloro-3-quinolylamino)-2-{3- [(dimethylamino)methyl]-2,3- dihydro-1,4-dioxa-5-aza-7- naphthylamino}pyrimidine |
| 178 | 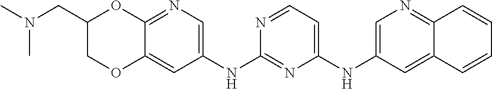 [SMILES: CN(C)CC1COc2cc(Nc3nccc(Nc4cnc5ccccc5c4)n3)cnc2O1] | 2-{3-[(dimethylamino)methyl]-2,3- dihydro-1,4-dioxa-5-aza-7- naphthylamino}-4-(3- quinolylamino)pyrimidine |
| 497 | 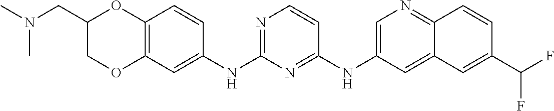 [SMILES: CN(C)CC1COc2cc(Nc3nccc(Nc4cnc5ccc(C(F)F)cc5c4)n3)ccc2O1] | 4-[6-(difluoromethyl)-3- quinolylamino]-2-{2- [(dimethylamino)methyl]-2,3- dihydro-1,4-benzodioxin-6- ylamino}pyrimidine |
| 498 | 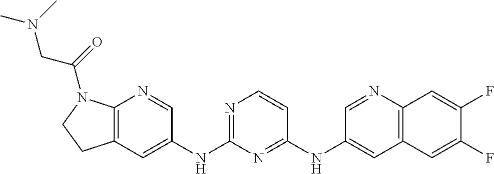 [SMILES: CN(C)CC(=O)N1CCc2cc(Nc3nccc(Nc4cnc5cc(F)c(F)cc5c4)n3)cnc21] | 1-{5-[4-(6,7-difluoro-3- quinolylamino)-2- pyrimidinylamino]- 1,7-diaza-1-indanyl}-2- (dimethylamino)-1-ethanone |
| 499 | 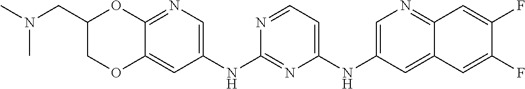 [SMILES: CN(C)CC1COc2cc(Nc3nccc(Nc4cnc5cc(F)c(F)cc5c4)n3)cnc2O1] | 4-(6,7-difluoro-3-quinolylamino)- 2-{3-[(dimethylamino)methyl]-2,3- dihydro-1,4-dioxa-5-aza-7- naphthylamino}pyrimidine |
| 500 | 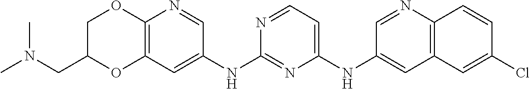 [SMILES: CN(C)CC1COc2ncc(Nc3nccc(Nc4cnc5ccc(Cl)cc5c4)n3)cc2O1] | 4-(6-chloro-3-quinolylamino)-2-{2- [(dimethylamino)methyl]-2,3- dihydro-1,4-dioxa-5-aza-7- naphthylamino}pyrimidine |
| 501 | 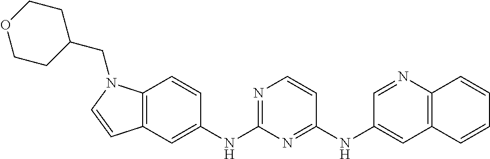 [SMILES: c1ccc2ncc(Nc3ccnc(Nc4ccc5c(ccn5CC5CCOCC5)c4)n3)cc2c1] | 4-(3-quinolylamino)-2-{1- [(tetrahydro-2H-pyran-4-yl)methyl]- 5-indolylamino}pyrimidine |
| 502 | 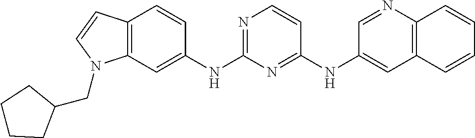 [SMILES: c1ccc2ncc(Nc3ccnc(Nc4ccc5ccn(CC6CCCC6)c5c4)n3)cc2c1] | 2-[1-(cyclopentylmethyl)-6- indolylamino]-4-(3- quinolylamino)pyrimidine |
| 503 | 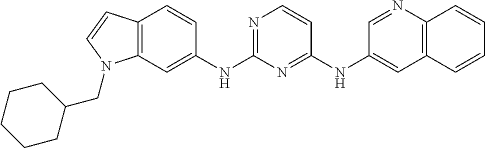 [SMILES: c1ccc2ncc(Nc3ccnc(Nc4ccc5ccn(CC6CCCCC6)c5c4)n3)cc2c1] | 2-[1-(cyclohexylmethyl)-6- indolylamino]-4-(3-quinolylamino)pyrimidine |
| 504 | 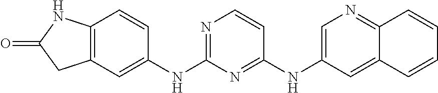 [SMILES: O=C1Cc2cc(Nc3nccc(Nc4cnc5ccccc5c4)n3)ccc2N1] | 5-[4-(3-quinolylamino)-2- pyrimidinylamino]-2-indolinone |
| 505 | 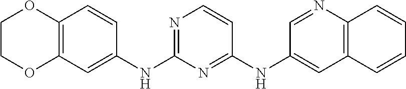 [SMILES: c1ccc2ncc(Nc3ccnc(Nc4ccc5c(c4)OCCO5)n3)cc2c1] | 2-(2,3-dihydro-1,4-benzodioxin-6- ylamino)-4-(3- quinolylamino)pyrimidine |

In some embodiments, the compound is a pharmaceutically acceptable salt of a compound described in Table 8.

Also provided herein is a compound selected from the Examples disclosed herein.

### Further Forms of Compounds

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

The term “acyl,” as used herein refers to the group —C(═O)—R, where R is alkyl, alkenyl, alkynyl, aryl, cycloalkyl, heteroaryl, heterocycle, or any other moiety were the atom attached to the carbonyl is carbon. An “acetyl” group refers to a —C(═O)CH₃ group.

The term “alkenyl,” as used herein refers to a straight-chain or branched-chain hydrocarbon radical having one or more double bonds and containing from 2 to 20 carbon atoms. In certain embodiments, alkenyl includes 2 to 6 carbon atoms. The term “alkenylene” refers to a divalent alkenyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkoxy” refers to a (alkyl)-O— group, where alkyl is as defined herein. In some embodiments, the alkoxy group is a C₁-C₆alkoxy, which refers to a (C₁-C₆alkyl)-O— group. Examples of alkyl groups include methoxy, ethoxy, n-propoxy, isopropoxy, n-butoxy, iso-butoxy, sec-butoxy, tert-butoxy, and the like.

An “alkyl” group refers to an aliphatic hydrocarbon group. In some embodiments, the alkyl is a straight-chain or branched-chain aliphatic hydrocarbon group containing from 1 to 20 carbon atoms. In certain embodiments, alkyl includes 1 to 10 carbon atoms. In further embodiments, the alkyl includes 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. In some embodiments, an alkyl is a C₁-C₆alkyl. In one aspect the alkyl is methyl, ethyl, propyl, iso-propyl, n-butyl, iso-butyl, sec-butyl, or t-butyl. The term “alkylene” refers to a divalent alkyl, such as methylene (—CH₂—). In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like.

The term “amino,” as used herein refers to —NRR′, wherein R and R′ are independently selected from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. In one aspect, “amino” as used herein refers to an —NH₂ group.

The term “alkylthio,” as used herein refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like. In some embodiments, the term “alkylthio” refers to the —S-(alkyl) group. In some embodiments, the alkylthio group is a C₁-C₆alkylthio, which refers to the —S—(C₁-C₆alkyl) group.

The term “alkynyl,” as used herein refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH. The term “alkynylene” refers to a carbon-carbon triple bond attached at two positions such as ethynylene (—C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The term “aromatic” refers to a planar ring having a delocalized it-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

The term “aryl” as used herein means a carbocyclic aromatic system containing one, two or three rings wherein such polycyclic ring systems are fused together. The term “aryl” embraces aromatic groups such as phenyl, naphthyl, anthracenyl, and phenanthryl. In one aspect, aryl is phenyl or a naphthyl. In some embodiments, an aryl is a phenyl. In some embodiments, an aryl is a phenyl, naphthyl, indanyl, indenyl, or tetrahyodronaphthyl. In some embodiments, an aryl is a C₆-C₁₀aryl. Depending on the structure, an aryl group is a monoradical or a diradical (i.e., an arylene group).

The term “aryloxy” as used herein refers to an aryl group attached to the parent molecular moiety through an oxy. In some embodiments, aryloxy as used herein refers to the aryl-O— group. In some embodiments, aryloxy is phenoxy, or a phenyl-O— group.

The terms “benzo” and “benz,” as used herein refer to fused bicyclic or polycyclic ring system that is formed with benzene as one of the rings. Examples include benzofuran, benzothiophene, and benzimidazole.

The term “cycloalkyl,” as used herein refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In some embodiments, cycloalkyl groups include groups having from 3 to 10 ring atoms. In certain embodiments, said cycloalkyl will comprise from 5 to 7 carbon atoms. In certain embodiments, said cycloalkyl will comprise from 3 to 6 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantly, and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane. In some embodiments, a cycloalkyl is a C₃-C₆cycloalkyl. In some embodiments, a cycloalkyl is a C₃-C₄cycloalkyl.

The term “cycloalkyloxy” as used herein refers to a cycloalkyl group attached to the parent molecular moiety through an oxy. In some embodiments, the cycloalkyloxy is a C₃-C₆cycloalkoxy, which refers to a (C₃-C₆cycloalkyl)-O— group.

A “cycloalkylalkyl” refers to an alkyl in which one hydrogen atom is replaced by a cycloalkyl group, as defined herein. In some embodiments, cycloalkylalkyl is a (C₃-C₆cycloalkyl)-C₁-C₆alkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. In some embodiments, the term “heteroaryl,” as used herein refers to a 3 to 15 membered unsaturated heteromonocyclic ring, or a fused monocyclic, bicyclic, or tricyclic ring system in which at least one of the fused rings is aromatic, which contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl, and the like. Exemplary tricyclic heterocyclic groups include carbazolyl, benzidolyl, phenanthrolinyl, dibenzofuranyl, acridinyl, phenanthridinyl, xanthenyl, and the like. In some embodiments, a heteroaryl contains 0-4 N atoms in the ring. In some embodiments, a heteroaryl contains 1-4 N atoms in the ring. In some embodiments, a heteroaryl contains 1 O atom in the ring. In some embodiments, a heteroaryl contains 1 S atom in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, the term “heterocycloalkyl” as used herein each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-2 N atoms in the ring. In some embodiments, a heterocycloalkyl contains 1-2 O atoms. In some embodiments, a heterocycloalkyl contains 1 S atom. In some embodiments, a heterocycloalkyl contains 0-2 N atoms, 0-2 O atoms and 0-1 S atoms in the ring.

The term “carbamate,” as used herein refers to an ester of carbamic acid (—NHCOO—) which may be attached to the parent molecular moiety from either the nitrogen or acid end, and which may be optionally substituted as defined herein.

The term “carboxyl” or “carboxy,” as used herein, refers to —C(═O)OH or the corresponding “carboxylate” anion, such as is in a carboxylic acid salt.

The term “cyano,” as used herein refers to —CN.

The term “ester,” as used herein refers to a carboxy group bridging two moieties linked at carbon atoms.

The term “ether,” as used herein refers to an oxy group bridging two moieties linked at carbon atoms.

The term “halo,” or “halogen,” as used herein refers to fluorine, chlorine, bromine, or iodine. In some embodiments, halo is fluoro, chloro, or bromo.

The term “haloalkyl,” as used herein refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—), and the like. In one aspect, a haloalkyl is a C₁-C₆haloalkyl. In another aspect, a haloalkyl is a C₁-C₄haloalkyl.

The term “haloalkoxy,” as used herein refers to a haloalkyl group attached to the parent molecular moiety through an oxygen atom. In one aspect, the haloalkoxy is a C₁-C₆haloalkoxy, which refers to a (C₁-C₆haloalkyl)-O— group. In another aspect, the haloalkoxy is a C₁-C₄haloalkoxy, which refers to a (C₁-C₄haloalkyl)-O— group.

The term “heteroalkyl” refers to an alkyl wherein 1 or more carbon atoms are replaced with a heteroatom. In some embodiments, “heteroalkyl” refers to an alkyl wherein 1 or more carbon atoms are replaced with one or more heteroatoms that are independently selected from NH, —N(alkyl), O, S, S(═O) and S(═O)₂. The attachment of the heteroatom(s) to the remainder of the compound is at a carbon atoms of the heteroalkyl. In some embodiments, up to two heteroatoms may be consecutive, such as, for example, —CH₂—NH—OCH₃. In some embodiments, “heteroalkyl” is an “alkoxyalkyl”, “alkylthioalkyl”, or “alkylaminoalkyl”. “Alkoxyalkyl” refers to an alkyl in which one hydrogen atom is replaced by an alkoxy group, as defined herein. In some embodiments, an alkoxyalkyl is a (C₁-C₆alkoxy)-C₁-C₆alkyl. Typical alkoxyalkyl groups include, but are not limited to, —CH₂OCH₃, —CH₂CH₂OCH₃, —CH₂CH₂CH₂OCH₃, —CH₂CH₂CH₂CH₂OCH₃, —CH₂OCH₂CH₃, —CH₂CH₂OCH₂CH₃, —CH₂CH₂CH₂OCH₂CH₃, —CH₂CH₂CH₂CH₂OCH₂CH₃, and the like. “Alkylthioalkyl” refers to an alkyl in which one hydrogen atom is replaced by an alkylthio group, as defined herein. In some embodiments, an alkoxyalkyl is a (C₁-C₆ alkylthio)-C₁-C₆alkyl. Typical alkoxyalkyl groups include, but are not limited to, —CH₂SCH₃, —CH₂CH₂SCH₃, —CH₂CH₂CH₂SCH₃, —CH₂CH₂CH₂CH₂SCH₃, —CH₂SCH₂CH₃, —CH₂CH₂SCH₂CH₃, —CH₂CH₂CH₂SCH₂CH₃, —CH₂CH₂CH₂CH₂SCH₂CH₃, and the like. “Alkylaminoalkyl” refers to an alkyl in which one hydrogen atom is replaced by an alkylamino group, as defined herein. In some embodiments, an alkoxyalkyl is a (C₁-C₆alkylamino)-C₁-C₆alkyl. Typical alkoxyalkyl groups include, but are not limited to, —CH₂NHCH₃, —CH₂CH₂NHCH₃, —CH₂CH₂CH₂NHCH₃, —CH₂CH₂CH₂CH₂NHCH₃, —CH₂NHCH₂CH₃, —CH₂CH₂NHCH₂CH₃, —CH₂CH₂CH₂NHCH₂CH₃, —CH₂CH₂CH₂CH₂NHCH₂CH₃, and the like.

The term “hydroxy,” or “hydroxyl,” as used herein refers to —OH.

The term “hydroxyalkyl,” as used herein refers to a hydroxy group attached to the parent molecular moiety through an alkyl group. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

The phrase “linear chain of atoms” refers to the longest straight chain of atoms independently selected from carbon, nitrogen, oxygen and sulfur.

The term “nitro,” as used herein refers to —NO₂.

The term “oxo,” as used herein refers to ═O.

The terms “sulfonate,” “sulfonic acid,” and “sulfonic,” as used herein refer the —SO₃H group and its anion as the sulfonic acid is used in salt formation.

The term “sulfanyl,” as used herein refers to —S—.

The term “sulfinyl,” as used herein refers to —S(═O)—.

The term “sulfonyl,” as used herein refers to a —S(═O)₂—, —S(═O)₂R, or —S(═O)₂R— group, with R as defined herein.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

In some embodiments, the term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention.

Additionally, the compounds disclosed herein can exist in unsolvated as well as “solvated” forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” or “disorder” as used herein refers to any condition that impairs the normal functioning of the body, such as a functional abnormality or disturbance that impairs normal functioning.

The term “combination therapy” means the administration of two or more therapeutic agents to treat a therapeutic condition or disorder described in the present disclosure.

“IGF-1R inhibitor” is used herein to refer to a compound that exhibits an IC₅₀ with respect to IGF-1R activity of no more than about 10 μM and more typically not more than about 5 μM, as measured in the IGF-1R assay described generally herein. “IC₅₀” is that concentration of inhibitor which reduces the activity of an enzyme (e.g., IGF-1R) to half-maximal level. Certain compounds disclosed herein have been discovered to exhibit inhibition against IGF-1R. In certain embodiments, compounds will exhibit an EC₅₀ with respect to IGF-1R of no more than about 2 μM; in yet further embodiments, compounds will exhibit an EC₅₀ with respect to IGF-1R of not more than about 1 μM; in yet further embodiments, compounds will exhibit an EC₅₀ with respect to IGF-1R of not more than about 500 nM, as measured in the IGF-1R assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease or may involve prevention of disease progression. Prevention of diseases may also mean prevention of progression of a disease to a later stage of the disease.

The term “patient” is generally synonymous with the term “subject” and includes all mammals including humans. Examples of patients include humans, non-human primates such as chimpanzees, and other apes and monkey species; livestock such as cows, goats, sheep, pigs, and rabbits, and companion animals such as dogs, cats, rabbits, and horses. Preferably, the patient is a human.

### Pharmaceutical Compositions and Formulations

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound provided herein in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro et. al.).

The compounds provided herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds provided herein or a salt, solvate, or hydrate thereof can be used as active ingredients in pharmaceutical compositions, specifically as IGF-1R inhibitors. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds provided herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds provided herein. Representative doses include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated, or prophylaxis conducted, or on whether further active compounds are administered in addition to the compounds provided herein and as part of a drug combination. The dosage regimen for treating a disease condition with the compounds and/or compositions provided herein is selected in accordance with a variety of factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods provided herein.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents.

Aqueous formulations suitable for oral use can be prepared by dissolving or suspending the active component in water and adding suitable colorants, flavors, stabilizing and thickening agents, as desired.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds provided herein may be formulated as ointments, creams, or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

### Indications and Methods of Treatment

Also provided herein are methods for treating IGF-1R-mediated disorders in a mammal in need of such treatment comprising administering to the mammal an amount of a compound disclosed herein effective to halt the further progression of, reduce the severity of, undo the effect of, or prevent the return of the disease or disorder in the subject. In a related aspect, certain embodiments provide therapeutic compositions comprising at least one compound disclosed herein in combination with one or more additional agents for the treatment of IGF-1R-mediated disorders.

Specific diseases to be treated by the compounds, compositions, and methods disclosed herein include thyroid eye disease (TED), also known as thyroid-associated ophthalmopathy (TAO), or Graves' ophthalmopathy or orbitopathy (GO).

Besides being useful for human treatment, certain compounds and formulations disclosed herein may also be useful for veterinary treatment of companion animals (e.g., horses, dogs, and cats), exotic animals farm animals, rodents, and the like.

Accordingly, the present invention also relates to a method of inhibiting at least one IGF-1R function comprising the step of contacting IGF-1R with a compound as described herein. The cell phenotype, cell proliferation, activity of IGF-1R, change in biochemical output produced by active IGF-1R, expression of IGF-1R, or binding of IGF-1R with a natural binding partner may be monitored. Such methods may be modes of treatment of disease, biological assays, cellular assays, biochemical assays, or the like.

Also provided herein is a method of treatment of a IGF-1R-mediated disease comprising the administration of a therapeutically effective amount of a compound as disclosed herein, or a salt thereof, to a patient in need thereof.

### Thyroid Eye Disease (TED)

As used herein, “Thyroid Eye Disease” (TED), “Thyroid-associated Ophthalmopathy” (TAO), “Thyroid Inflammatory Eye Disease (TIED),” “Graves' Ophthalmopathy” (GO) or “Graves' Orbitopathy” (GO) refer to the same disease or disorder. These terms refer to the inflammatory orbital pathology associated with some autoimmune thyroid disorders, most commonly with “Graves' Disease” (GD), but sometimes with other diseases, e.g. Hashimoto's thyroiditis.

The cause of TED is unknown. TED is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos.

The annual incidence rate of TED has been estimated at 16 cases per 100,000 women and 2.9 cases per 100,000 men from a study based in one largely rural Minnesota community. There appears to be a female preponderance in which women are affected 2.5-6 times more frequently than men; however, severe cases occur more often in men than in women. In addition, most patients are aged 30-50 years, with severe cases appearing to be more frequent in those older than 50 years. Although most cases of TED do not result in loss of vision, this condition can cause vision-threatening exposure keratopathy, troublesome diplopia (double vision), and compressive dysthyroid optic neuropathy.

In some embodiments, TED precedes, coincides with, or follows the systemic complications of dysthyroidism. The ocular manifestations of TED include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences.

Many of the signs and symptoms of TED, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibers, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-1β, interferon-γ, platelet-derived growth factor, thyroid stimulating hormone (TSH), and insulin-like growth factor 1 (IGF-1).

TED is commonly considered to be the autoimmune orbital manifestation of Graves' Disease (GD). However, only approximately 30% of patients with Graves' hyperthyroidism manifest clinically relevant ocular pathology indicating there is mechanistic heterogeneity and differentiation between the conditions. The molecular mechanisms underlying TED remain unclear. It is accepted that the generation of autoantibodies that act as agonists on the thyroid-stimulating hormone receptor (TSHR) is responsible for Graves' hyperthyroidism. Pathogenic overstimulation of TSHR, leads to overproduction of thyroid hormones (T3 and T4) and accelerated metabolism of many tissues.

Antibodies that activate the insulin-like growth factor 1 receptor (IGF-1R) have also been detected and implicated in active TED. Without being bound to any theory, it is believed that TSHR and IGF-1R form a physical and functional complex in orbital fibroblasts, and that blocking IGF-1R appears to attenuate both IGF-1 and TSH-dependent signaling. It has been suggested that blocking IGF-1R using an antibody antagonist might reduce both TSHR- and IGF-1-dependent signaling and therefore interrupt the pathological activities of autoantibodies acting as agonists on either receptor.

Immunoglobulins that activate IGF-1R signaling have been detected in patients with GD and TED. Furthermore, IGF-1 synergistically enhances the actions of thyrotropin. IGF-1R, a membrane-spanning tyrosine kinase receptor with roles in development and metabolism, also stimulates immune function and thus might be targeted therapeutically in autoimmune diseases. IGF-1R is overexpressed by orbital fibroblasts and by T cells and B cells in persons with GD and TED. It forms a signaling complex with TSHR through which it is transactivated. In vitro studies of orbital fibroblasts and fibrocytes show that IGF-1R-inhibitory antibodies can attenuate the actions of IGF-1, thyrotropin, thyroid-stimulating immunoglobulins, and immunoglobulins isolated from patients with GD and TED. These observations prompted a trial of teprotumumab, a fully human IGF-1R-inhibitory monoclonal antibody, in patients with active, moderate-to-severe TED.

The terms “proptosis” and “exophthalmos” (also known as exophthalmus, exophthalmia, or exorbitism) refer to the forward projection, displacement, bulging, or protrusion of an organ. As used herein, the terms refer to the forward projection, displacement, bulging, or protrusion of the eye anteriorly out of the orbit. Proptosis and exophthalmos are considered by some of skill in the art to have the same meaning and are often used interchangeably, while others attribute subtle differences to their meanings. Exophthalmos is used by some to refer to severe proptosis; or to refer to endocrine-related proptosis. Yet others use the term exophthalmos when describing proptosis associated with the eye, in, for example, subjects with TED (TAO or GO).

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases & extra orbital lesions. TED (TAO or GO) is currently recognized as the most common cause of proptosis in adults. Exophthalmos can be either bilateral, as is often seen in TED (TAO or GO), or unilateral (as is often seen in an orbital tumor).

Measurement of the degree of exophthalmos can be performed using an exophthalmometer, an instrument used for measuring the degree of forward displacement of the eye. The device allows measurement of the forward distance of the lateral orbital rim to the front of the cornea.

Computed tomography (CT) scanning and Magnetic resonance imaging (MRI) may also be used in evaluating the degree of exophthalmos or proptosis. CT scanning is an excellent imaging modality for the diagnosis of TED (TAO or GO). In addition to allowing visualization of the enlarged extraocular muscles, CT scans provide the surgeon or clinician with depictions of the bony anatomy of the orbit when an orbital decompression is required. MRI, with its multi-planar and inherent contrast capabilities, provides excellent imaging of the orbital contents without the radiation exposure associated with CT scan studies. MRI provides better imaging of the optic nerve, orbital fat, and extraocular muscle, but CT scans provide better views of the bony architecture of the orbit.

Orbital ultrasonography can also be a used for the diagnosis and evaluation of TED (TAO or GO), because it can be performed quickly and with a high degree of confidence. High reflectivity and enlargement of the extraocular muscles are assessed easily, and serial ultrasonographic examinations can also be used to assess progression or stability of the ophthalmopathy.

Based on the technologies currently available, or that will become available in the future, one of skill in the art would be capable of determining the best modality for diagnosing and evaluating the extent of proptosis or exophthalmos.

Although it is generally accepted that the normal range of proptosis is 12-21 mm, it must be noted that the value for a normal person varies by age, gender and race. For example, in normal adult white males, the average distance of globe protrusion is 16.5 mm, with the upper limit of normal at 21.7 mm. In adult African Americans it averages 18.2 mm, with an upper normal limit of 24.1 mm in males and 22.7 mm in females. In Mexican adults, males averaged 15.2 mm and females averaged 14.8 mm and in Iran, for the age group of 20-70 years, the average was 14.7 mm. In Taiwanese adults, comparing normal subjects to those with Graves' Ophthalmopathy, the normal group had an average reading of 13.9 mm versus 18.3 mm for the TED group.

Even within a group of people, there can be variability. Four ethnic groups in Southern Thailand had exophthalmometry measurement averages ranging from 15.4 mm to 16.6 mm. In 2477 Turkish patients, the median measurement was 13 mm, with an upper limit of 17 mm; and in a Dutch study, the upper limit was 20 mm in males and 16 mm in females.

Although the average and upper limits for exophthalmos or proptosis vary widely, it is accepted in the field that a difference greater than 2 mm between the eyes is significant and not normal.

One of skill in the art, for example an ophthalmologist, surgeon or other clinician skilled in the knowledge and treatment of eye disorders would know what a normal value of proptosis is based on the age, gender and race of the subject and have the ability to diagnose or evaluate the presence or absence of proptosis as well as track its progression.

### Activity Measures or Assessments

Several classification systems have been conceived to assess the clinical manifestations of TED (TAO or GO). In 1969, Werner reported the NOSPECS Classification (No physical signs or symptoms, Only signs, Soft tissue involvement, Proptosis, Extraocular muscle signs, Corneal involvement, and Sight loss) (Werner, S. C. American Journal of Ophthalmology, 1969, 68, no. 4, 646-648.)

The modified NOSPECS was also published by Werner in 1977 and has been broadly used since then (Werner, S. C. American Journal of Ophthalmology, 1977, 83, no. 5, 725-727). This classification grades for clinical severity and does not provide a means of distinguishing active TED (inflammatory progressive) from inactive TED (noninflammatory stationary). Therefore, the indication for treatments used to be based exclusively in the severity of symptoms without consideration whether the disease was active or inactive. In 1989, Mourits et al. described the Clinical Activity Score (CAS) (Mourits et al., British Journal of Ophthalmology, 1989, 73, no. 8, 639-644) as a way of assessing the degree of active disease. This score, based on the classical signs of acute inflammation (pain, redness, swelling, and impaired function) was proposed as a clinical classification to discriminate easily between active and inactive disease and was modified in 1997 (Mourits et al., Clinical Endocrinology, 1997. 47, no. 1, 9-14). This protocol is further described below.

As used herein, the term CAS refers to the protocol described and scored as disclosed below. According to this protocol, one point is given for the presence of each of the parameters assessed in the list below. The sum of all points defines clinical activity and provides the CAS. For patients assessed for the first time only items 1-7 are scored. A CAS≥3/7 indicates active GO. For patients that are assessed for the second or subsequent time (typically, 1-3 months later), items 8-10 are also scored; and a CAS≥4/10 indicates active disease. A ten-item CAS scale exists as well, but in clinical trials, the 7-item scale is generally used, being more amenable to longitudinal studies involving multiple assessments.

The CAS consists of seven components:
1. spontaneous retrobulbar pain,
2. pain on attempted eye movements (upward, side-to-side, and downward gazes),
3. conjunctival redness,
4. redness of the eyelids,
5. chemosis (conjunctival swelling/edema),
6. swelling of the caruncle/plica, and
7. swelling of the eyelids.

Each component is scored as present (1 point) or absent (0 points). The score at each efficacy assessment is the sum of all items present; giving a range of 0-7, where 0 or 1 constitutes inactive disease and 7 severe active ophthalmopathy. A change of >2 points is considered clinically meaningful.

Item 1, spontaneous orbital pain could be a painful, or oppressive feeling on, or behind, the globe. This pain may be caused by the rise in intraorbital pressure, when the orbital tissues volume increases through excess synthesis of extracellular matrix, fluid accumulation, and cellular infiltration and expansion. Item 2, gaze evoked orbital pain, could be pain in the eyes when looking, or attempting to look, up, down or sideways, i.e., pain with upward, downward, or lateral eye movement, or when attempting upward, downward, or lateral gaze. This kind of pain could arise from the stretching of the inflamed muscle(s), especially on attempted up-gaze. The ‘stretching pain’ cannot be provoked by digital pressing on the eyeball, as would be expected if it were a manifestation of the raised intraorbital pressure. Both kinds of pain can be reduced after anti-inflammatory treatment. These kinds of pain are therefore considered to be directly related to autoimmune inflammation in the orbit and thus useful in assessing TED activity.

Swelling in TED (TAO or GO) is seen as chemosis (edema of the conjunctiva) and swelling of the caruncule and/or plica semilunaris. Both are signs of TED activity. Swollen eyelids can be caused by edema, fat prolapse through the orbital septum, or fibrotic degeneration. In addition to swelling, other symptoms indicative of active TED include redness and/or pain of the conjunctiva, eyelid, caruncule and/or plica semilunaris.

Other grading systems have also been developed for the assessment of TED (TAO or GO). The VISA Classification (vision, inflammation, strabismus, and appearance) (Dolman, P. J., and Rootman, J., Ophthalmic Plastic and Reconstructive Surgery, 2006, 22, no. 5, 319-324 and Dolman, P. J., Best Practice & Research Clinical Endocrinology & Metabolism, 2012, 26, no. 3, 229-248) and the European Group of Graves' Orbitopathy (EUGOGO) Classification (Bartalena, L., et al., European Journal of Endocrinology, 2008, 158, no. 3, 273-285) are two such examples. Both systems are grounded in the NO SPECS and CAS classifications and use indicators to assess the signs of activity and the degree of severity. More importantly, they allow the clinician to guide the treatment of the patient with GO. VISA is more commonly used in North America and Canada while EUGOGO is in Europe. Since the VISA and EUGOGO protocols are not interchangeable, only one of them should be employed as a reference in a specific patient.

### Graves Ophthalmopathy Quality of Life (GO-QoL)

In addition to proptosis (or exophthalmos) and CAS, quality of life (QoL) was also evaluated with the use of the Graves' ophthalmopathy quality of life (GO-QoL) questionnaire. This questionnaire is designed to determine the improved quality of life after treatment. In some embodiments, the questionnaire determines the decreased or lack of side effects after being treated with compound disclosed herein, or a pharmaceutically acceptable salt or solvate thereof, according to the methods disclosed herein, as compared to treatment with glucocorticoids.

The GO-QoL questionnaire has two self-assessment subscales. The first relates to the impact of visual function on daily activities, while the second relates to the impact of self-perceived appearance. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

### Severity Measures

For lid aperture, the distance between the lid margins are measured (in mm) with the patient looking in the primary position, sitting relaxed, and with distant fixation. For swelling of the eyelids, the measure/evaluation is either “absent/equivocal,” “moderate,” or “severe.” Redness of the eyelids is either absent or present. Redness of the conjunctivae is either absent or present. Conjunctival edema is either absent or present. Inflammation of the caruncle or plica is either absent or present. Exophthalmos was measured in millimeter using the same Hertel exophthalmometer and same intercanthal distance for an individual patient. Subjective diplopia is scored from 0 to 3 (0=no diplopia; 1=intermittent, i.e., diplopia in primary position of gaze, when tired or when first awakening; 2=inconstant, i.e., diplopia at extremes of gaze; 3=constant, i.e., continuous diplopia in primary or reading position). For eye muscle involvement, the ductions are measured in degrees. Corneal involvement is either absent/punctate or keratopathy/ulcer. For optic nerve involvement, .i.e., best-corrected visual acuity, color vision, optic disc, relative afferent pupillary defect, the condition is either absent or present. In addition, visual fields are checked if optic nerve compression was suspected.

### Severity Classification

Sight-threatening thyroid eye disease: Patients with dysthyroid optic neuropathy (DON) and/or corneal breakdown. This category warranted immediate intervention.

Moderate-to-severe thyroid eye disease: Patients without sight-threatening disease whose eye disease had sufficient impact on daily life to justify the risks of immunosuppression (if active) or surgical intervention (if inactive). Patients with moderate-to-severe thyroid eye disease usually had any one or more of the following: lid retraction ≥2 mm, moderate or severe soft tissue involvement, exophthalmos ≥3 mm above normal for race and gender, inconstant or constant diplopia.

Mild thyroid eye disease: Patients whose features of thyroid eye disease have only a minor impact on daily life insufficient to justify immunosuppressive or surgical treatment. They usually have only one or more of the following: minor lid retraction (<2 mm), mild soft tissue involvement, exophthalmos <3 mm above normal for race and gender, transient or no diplopia, and corneal exposure responsive to lubricants.

### Assessment of Gorman Grading of Diplopia

The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of ≥1 grade is considered clinically meaningful.

Additional testing, including clinical trial protocols and criteria and the lead-in study, which can be performed to determine efficacy for the treatment of TED can be found in US20190225696A1, which is hereby incorporated by reference in its entirety.

In some embodiments, the IGR-1R inhibitors described herein treat TED in subjects who were either proptosis non-responders (<2 mm reduction in proptosis in the study eye) in the lead-in study or were proptosis responders in the lead-in study but meet the criteria for re-treatment due to relapse.

### Methods of Treatment

Accordingly, provided herein are methods for treating IGF-1R-mediated disorders in a mammal in need of such treatment comprising administering to said subject an amount of a compound disclosed herein effective to reduce or prevent said disorder in the subject. In a related aspect, certain embodiments provide therapeutic compositions comprising at least one compound disclosed herein in combination with one or more additional agents for the treatment of IGF-1R-mediated disorders. Specific diseases to be treated by the compounds, compositions, and methods disclosed herein include thyroid eye disease (TED), also known as thyroid-associated ophthalmopathy (TAO), or Graves' ophthalmopathy or orbitopathy (GO).

In some embodiments, the disease is thyroid eye disease (TED).

Also provided herein is a compound disclosed herein, or a pharmaceutically acceptable salt or solvate thereof, for use as a medicament.

Also provided herein is a compound disclosed herein, or a pharmaceutically acceptable salt or solvate thereof, for use as a medicament for the treatment of a IGF-1R-mediated disease.

Also provided is a compound disclosed herein, or a pharmaceutically acceptable salt or solvate thereof, for use in the manufacture of a medicament for the treatment of a IGF-1R-mediated disease.

Also provided is the use of a compound disclosed herein, or a pharmaceutically acceptable salt or solvate thereof, as disclosed herein for the treatment of a IGF-1R-mediated disease.

Also provided herein is a method of inhibition of IGF-1R comprising contacting IGF-1R with a compound as disclosed herein, or a pharmaceutically acceptable salt or solvate thereof.

### Combination Therapy

In certain instances, it may be appropriate to administer at least one of the compounds described herein (or a pharmaceutically acceptable salt, ester, or prodrug thereof) in combination with another therapeutic agent.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:

The invention is further illustrated by the following examples. All IUPAC names were generated CDD Vault from Collaborative Drug Discovery (Burlingame, CA). CDD Vault uses ChemDoodle (from iChem Labs) for the generation of IUPAC names. Currently, the version used within CDD Vault is ChemDoodle ver 2.8.0.

### I. Chemical Synthesis

### List of Commonly Used Abbreviations

Ac (acetyl);
ACN, CH₃CN (acetonitrile);
AcOH, HOAc (acetic acid);
atm (atmosphere);
BH₃-DMS complex, BMS (borane dimethylsulfide);
BINAP (2,2′-bis(diphenylphosphino)-1,1′-binaphthyl);
Boc (tert-butoxycarbonyl);
Boc₂O (di-tert-butyl dicarbonate);
BuLi, n-BuLi (n-buyllithium);
DCM (dichloromethane);
DIPEA (diisopropylethylamine);
DMAP (4-dimethylaminopyridine);
DME (dimethyl ether);
DMF (N,N-dimethylformamide);
DMSO (dimethylsulfoxide);
equiv. (equivalents);
Et (ethyl);
Et₃N, TEA (triethylamine);
EtOAc (ethyl acetate);
EtOH (ethanol);
g (grams); mg (milligrams); μg (micrograms);
h, hr, hrs (hours);
HCl (hydrochloric acid);
HFIP (hexafluoro-2-propanol, hexafluoroisopropanol);
HPLC (high-performance liquid chromatography);
IGF-1R (insulinlike growth factor1 receptor);
i-Pr (isopropyl);
i-PrOH, IPA (isopropanol);
L (liters); mL (milliliters); μL (microliters);
LAH (lithium aluminum hydride);
LC-MS (liquid chromatography-mass spectrometry);
LDA (lithium diisopropylamide);
M (molar); mM (millimolar); μM (micromolar);
Me (methyl);
MeI (methyl iodide, iodomethane);
MeLi (methyllithium);
MeOH (methanol);
min, mins (minute(s));
mol (moles); mmol (millimoles); gmol (micromoles);
MS (mass spectrometry);
MsCl (methanesulfonyl chloride, mesyl chloride);
N₂ (g) (nitrogen gas);
NaOtBu (sodium tert-butoxide);
NBS (N-bromosuccinimide);
NMP (N-methyl-2-pyrrolidine)
NMR (nuclear magnetic resonance);
Pd(OAc)₂ (palladium(II) acetate);
Pd₂(dba)₃ (tris(dibenzylideneacetone)dipalladium(0));
PE (petroleum ether);
PhMe (toluene);
Pr (propyl);
psi (pounds per square inch);
PTFE (polytetrafluoroethylene, Teflon®);
PyBrop (bromo-tris-pyrrolidino-phosphonium hexafluorophosphate);
RBF (round bottom flask);
Rt, RT (room temperature);
SiO₂ (silica);
SOCl₂ (thionyl chloride)
TBABr (tetrabutylammonium bromide);
t-Bu (tert-butyl);
tBu-Josiphos ((R)-(−)-1-[(S)-2-(dicyclohexyl-phosphino)ferrocenyl]ethyl-di-t-butyl phosphine);
tBuOH (tert-butyl alcohol, tert-butanol);
TCFH (N′-tetramethylformamidinium hexafluorophosphate);
THF (tetrahydrofuran);
TLC (thin layer chromatography);
μW (microwave);
vol. (volume equivalents);
Xantphos (4,5-Bis(diphenylphosphino)-9,9-dimethylxanthene).

### Synthetic Methods

The following intermediates and examples can be synthesized using the general synthetic procedures set forth in the following Examples.

### Example 1: General Procedure A

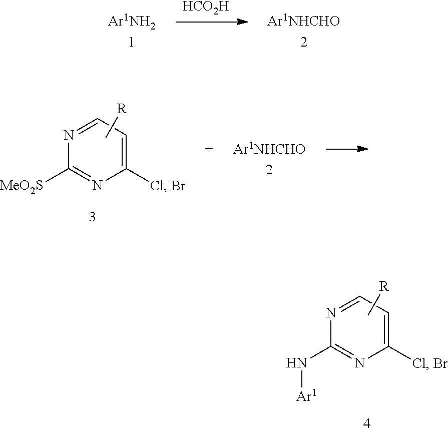

Step 1: A mixture of an appropriately substituted aryl or heteroaryl aniline 1 (1 equiv.) and formic acid (0.5-1 M) was heated at 100° C. until LC-MS analysis showed full conversion to the corresponding formamide. Formic acid was removed under reduced pressure. The residue was diluted with EtOAc and poured into K₂CO₃ (10% aq.) and the layers were separated. The organic layer was dried over Na₂SO₄ and concentrated to afford the desired product 2.

Step 2: To a solution of the formamide 2 (1 equiv.) and an appropriately substituted 4-halo-2-(methylsulfonyl)pyrimidine (1.2-1.5 equiv.) 3 in THF (0.2-0.5 M) was added a base such as sodium tert-butoxide or sodium tert-pentoxide (2-3 equiv.) portionwise over 5 mins and the reaction was stirred until LC-MS analysis showed the reaction was complete. The reaction mixture was diluted with NaOH 1 M aq. and stirred for 1 h. The mixture was extracted with EtOAc, dried, and concentrated. The crude product was purified by flash chromatography (SiO₂, eluting with CH₂Cl₂/MeOH/NH₄OH) to afford 4.

### Example 2: General Procedure B

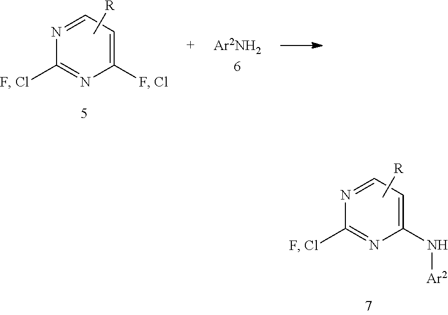

To a solution of an appropriately substituted halopyrimidine 5 (1 equiv.) and substituted aryl or heteroaryl aniline 6 (1.2 equiv.) in a solvent chosen from iPrOH, EtOH, THF, DMF, or NMP (0.2 M) was added either DIPEA, HCl, TFA or AcOH (5 equiv.). The reaction mixture was heated at a temperature ranging from 60-120° C. until LC-MS analysis showed the reaction was complete. The mixture was neutralized first with NH₄OH and concentrated to dryness. Purification was performed either by flash chromatography (SiO₂, eluting with CH₂Cl₂/MeOH/NH₄OH) or by reversed phase flash chromatography (C18, eluting with 10 mM ammonium bicarbonate or 10 mM ammonium formate in MeCN) to afford the desired product 7.

### Example 3: General Procedure C

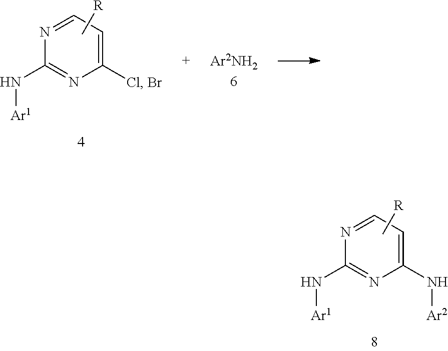

To a solution of an appropriately substituted halopyrimidine 4 (1 equiv.) and substituted aryl or heteroaryl aniline 6 (1.2 equiv.) in a solvent chosen from iPrOH, EtOH, THF, DMF or NMP (0.2 M) was added either HCl, TFA or AcOH (5 equiv.). The reaction mixture was heated at a temperature ranging from 60-120° C. until LC-MS analysis showed the reaction was complete. The mixture was neutralized with NH₄OH and concentrated to dryness. Purification was performed either by flash chromatography (SiO₂, eluting with CH₂Cl₂/MeOH/NH₄OH) or by reversed phase flash chromatography (C18, eluting with 10 mM ammonium bicarbonate or 10 mM ammonium formate in MeCN) to afford the desired product 8.

### Example 4: General Procedure D

A mixture of an appropriately substituted halopyrimidine 4 (1 equiv.), substituted aryl or heteroaryl aniline 6 (1.2 equiv.), a palladium source chosen from Pd(OAc)₂ or Pd₂(dba)₃ (0.1 equiv Pd), a ligand chosen from BINAP, Xantphos or DPEphos (0.1 equiv.) and Cs₂CO₃ (0.1 equiv.) in dioxane (1.0 mL) was purged with N₂. The reaction mixture was heated at a temperature ranging from 60-100° C. until LC-MS analysis showed the reaction was complete. The reaction was diluted with EtOAc/MeOH and filtered. The filtrate was concentrated and purified either by flash chromatography (SiO₂, eluting with CH₂Cl₂/MeOH/NH₄OH) or by reversed phase flash chromatography (C18, eluting with 10 mM ammonium bicarbonate or 10 mM ammonium formate in MeCN) to afford the desired product 8.

### Example 5: General Procedure E

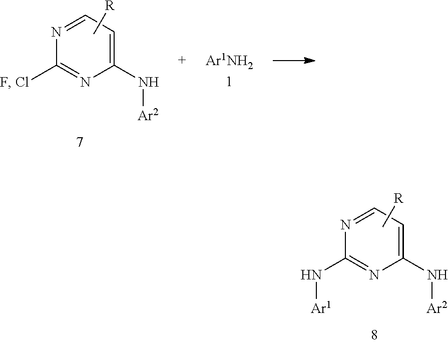

To a solution of an appropriately substituted haloppyrimidine 7 (1 equiv.) and substituted aryl or heteroaryl aniline 1 (1.2 equiv.) in a solvent chosen from iPrOH, EtOH, THF, DMF or NMP (0.2 M) was added either HCl, TFA or AcOH (5 equiv.). The reaction mixture was heated at a temperature ranging from 60-120° C. until LC-MS analysis showed the reaction was complete. The mixture was neutralized with NH₄OH and concentrated to dryness. Purification was performed either by flash chromatography (SiO₂, eluting with CH₂Cl₂/MeOH/NH₄OH) or by reversed phase flash chromatography (C18, eluting with 10 mM ammonium bicarbonate or 10 mM ammonium formate in MeCN) to afford the desired product 8.

### Example 6: General Procedure F

A mixture of an appropriately substituted haloppyrimidine 7 (1 equiv.), substituted aryl or heteroaryl aniline 1 (1.2 equiv.), Pd(OAc)₂ (0.1 equiv.), BINAP (or XPhos, XantPhos) (0.1 equiv.) and Cs₂CO₃ (0.1 equiv.) in Dioxane (1.0 mL) was purged with N₂. The reaction mixture was heated at a temperature ranging from 60-100° C. until LC-MS analysis showed the reaction was complete. The reaction was diluted with EtOAc/MeOH and filtered. The filtrate was concentrated and purified either by flash chromatography (SiO₂, eluting with CH₂Cl₂/MeOH/NH₄OH) or by reversed phase flash chromatography (C18, eluting with 10 mM ammonium bicarbonate or 10 mM ammonium formate in MeCN) to afford the desired product 8.

### Example 7: General Procedure G

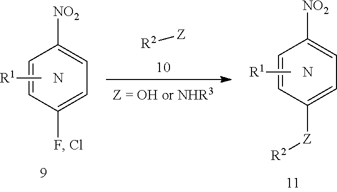

To a solution of an appropriately substituted para halo nitro aryl or heteroaryl 9 (1 equiv.) and amine or alcohol 10 (1.2-1.5 equiv.) in a solvent chosen from DMF, NMP, DMSO, THF or Dioxane (0.2-0.5 M) was added a base such as K₂CO₃, Cs₂CO₃, Et₃N, DIPEA, NaH or NaOtBu (1.2-1.5 equiv.) and the reaction was stirred at RT or heated at 50-100° C. until LC-MS analysis showed the reaction was complete. The reaction mixture was extracted with EtOAc, dried, and concentrated to afford the desired product 11.

### Example 8: General Procedure H

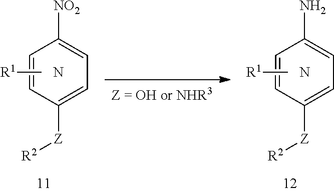

A mixture of the nitro aryl or heteroaryl 11 (1 equiv.) and 10% Pd/C (0.1 equiv.) in MeOH or EtOH (0.2-0.5 M) was hydrogenated under an atmosphere of hydrogen until LC-MS analysis showed the reaction was complete. The reaction mixture was filtered through celite and the volatiles were evaporated to afford the desired product 12.

### Example 9: General Procedure I

A mixture of the nitro aryl or heteroaryl 11 (1 equiv.) in EtOH and sat. NH₄Cl (3:1, 0.2-0.5 M) was added iron powder (or zinc powder) (4-10 equiv.) and the mixture was heated at 60-80° C. until LC-MS analysis showed the reaction was complete. The volatiles were evaporated the residue was slurried with EtOAc and filtered through Celite. The filtrate was washed with water, dried, and concentrated to dryness to afford the desired product 12.

### Example 10: General Procedure J

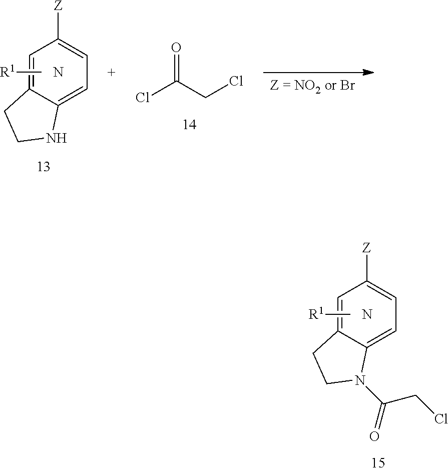

To a mixture of indoline derivative 13 (1 equiv.) and DIPEA (3 equiv.) in CH₂Cl₂ (0.2 M) at 0° C. was added dropwise chloroacetyl chloride 14 (2 equiv.). The reaction mixture was stirred at 0° C. for 15 mins, and then at room temperature until LC-MS analysis showed completion of reaction. The reaction mixture was diluted with sat. NaHCO₃. The mixture was extracted with EtOAc, washed with brine, dried, and concentrated to dryness to afford the chloro amide 15.

### Example 11: General Procedure K

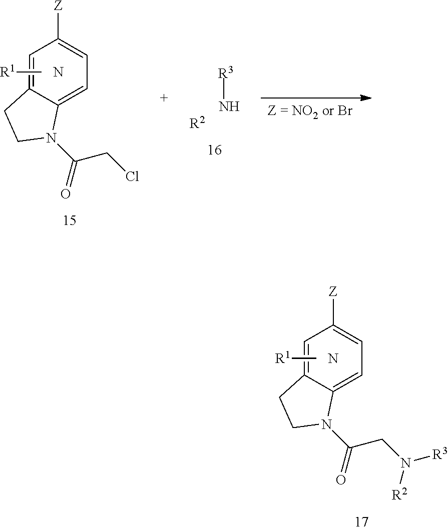

To a mixture of the chloro amide 15 (1 equiv.) and K₂CO₃ (2.6 equiv.) in PhMe (0.5 M) was added amine nucleophile 16 (3 equiv.). The reaction mixture was heated at 70° C. until LC-MS analysis showed the reaction was complete. The reaction mixture was filtered and concentrated to dryness. The crude product was purified by flash chromatography (SiO₂, eluting with CH₂Cl₂/MeOH/NH₄OH) to afford the desired product 17.

### Example 12: General Procedure L

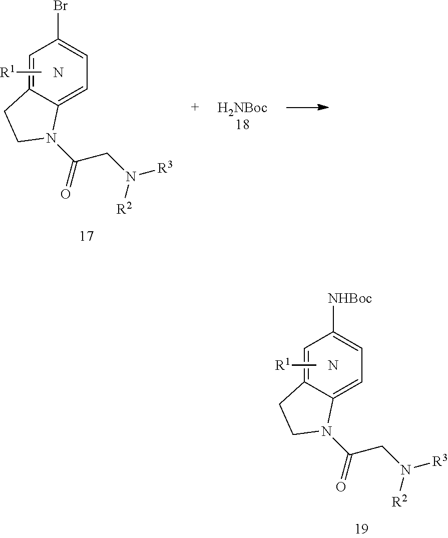

A mixture of bromoarene 17 (1 equiv.), tert-butylcarbamate 18 (1.2 equiv.), Pd(OAc)₂ (0.1 equiv.), BINAP (or XPhos, XantPhos) (0.1 equiv.) and Cs₂CO₃ (0.1 equiv.) in dioxane (1.0 mL) was purged with N₂. The reaction mixture was heated at a temperature ranging from 60-100° C. until LC-MS analysis showed the reaction was complete. The reaction was diluted with EtOAc/MeOH and filtered. The filtrate was concentrated and purified either by flash chromatography (SiO₂, eluting with CH₂Cl₂/MeOH/NH₄OH) or by reversed phase flash chromatography (C18, eluting with 10 mM ammonium bicarbonate or 10 mM ammonium formate in MeCN) to afford the desired product 19.

### Example 13: General Procedure M

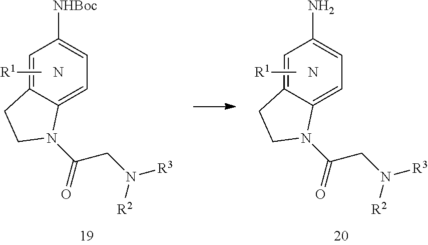

A mixture of carbamate 19 (1 equiv.) and trifluoroacetic acid (20 equiv.) in CH₂Cl₂ (0.05 M) was stirred until LC-MS analysis showed the reaction was complete. The reaction was concentrated to dryness to afford the desired product 20.

### Example 14: General Procedure N

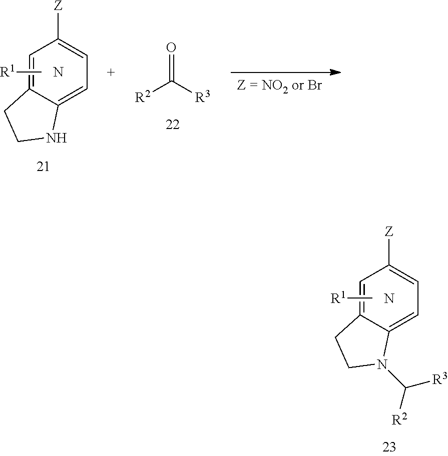

To a mixture of indoline derivative 21 (1 equiv.) MeOH (0.1 M) was added carbonyl 22 (2 equiv.), acetic acid (5 equiv.), and sodium cyanoborohydride (2 equiv.). The reaction mixture was stirred until LC-MS analysis showed completion of reaction. The reaction mixture was diluted with NaOH (6 M aq.) and then concentrated to dryness. The crude mixture was redissolved in water and then extracted with CH₂Cl₂, dried, and concentrated. The crude product was purified by flash chromatography (SiO₂, eluting with CH₂Cl₂/MeOH/NH₄OH) to afford the desired product 23.

### Example 15: General Procedure O

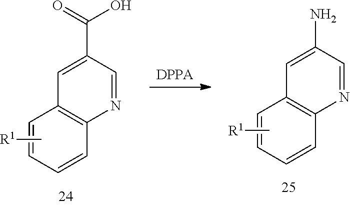

To a solution of quinoline-3-carboxylic acid 24 (1.0 equiv.) in toluene (0.05M) was added triethylamine (1.2 equiv.) and diphenyl phosphoryl azide (1.0 equiv.), and the reaction mixture was heated to reflux for 3 h. After cooling to 0° C., potassium trimethylsilanolate (2.0 equiv.) was added and the mixture was stirred for 16 h at room temperature. Water was added and stirred for 30 min. After quenching with 5% citric acid (5 mL), the mixture was then washed with Et₂O (2 times). The remaining aqueous solution was basified with 1 N NaOH, and then extracted with CH₂Cl₂. The combined CH₂C₁₂ extracts were washed with brine, dried, and concentrated to deliver the final product 25.

### Example 16: General Procedure P

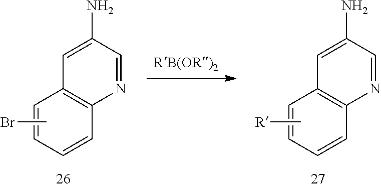

Bromoquinolin-3-amine 26 (1.0 equiv.), boronic ester (1.2 equiv.), tricyclohexylphosphine (0.2 equiv.), palladium(II) acetate (0.1 equiv.), and potassium phosphate tribasic (2.5 equiv.) in toluene and water (0.2 M, 10:1), was heated to 110° C. for 24 hours. The crude reaction was cooled and filtered through celite (EtOAc) and purified by flash column chromatography (SiO₂, eluting with EtOAc/hexanes) to afford the title product 27.

### Example 17: General Procedure Q

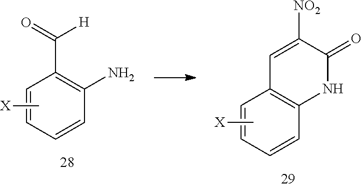

To 2-amino-benzaldehyde 28 (1.0 equiv.) dissolved in 1:1 mixture of H₂O and AcOH (0.25 M) and purged with nitrogen was added ethyl nitroacetate (3.0 equiv.) and the reaction was heated at 70° C. for 16 h. The precipitate was isolated by filtration, washed with cold H₂O, and dried to deliver the title product 29. The product be purified by flash column chromatography (SiO₂, eluting with EtOAc/hexanes) as needed.

### Example 18: General Procedure R

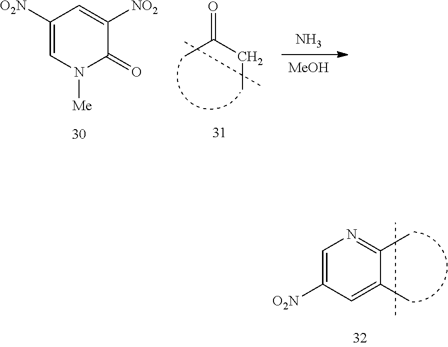

To a 0.2 to 0.4 M solution of 1-methyl-3,5-dinitropyridin-2(1H)-one (30, 1 equiv.) in methanolic ammonia (1 to 7 M). Cyclic ketone 31 (1 to 1.5 equiv.) was added and the mixture was tightly sealed and heated at 80±20° C. for 3 to 24 hours. The reaction was cooled and diluted with either Et₂O or EtOAc then washed with water. The organic layer was dried and concentrated. The residue was either used directly in the next step or, purified by flash chromatography (SiO₂, eluting with EtOAc/hexane) as needed to afford the desired product 32.

### Example 19: General Procedure S

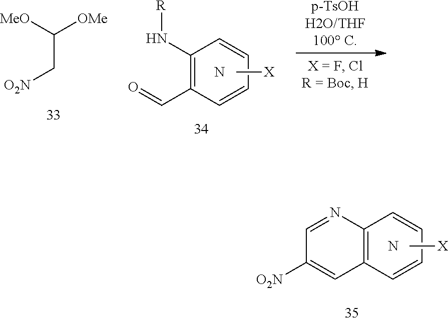

### Step 1: Synthesis of 1,1-dimethoxy-2-nitroethane

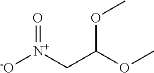

A solution of trimethylorthoformate (150 mL, 1.37 mol), nitromethane (371 mL, 6.84 mol), and zinc chloride (9.42 mL, 68.4 mmol) was heated to 120° C. After 44 hours the mixture was cooled and filtered through celite and purified by distillation (75° C., 12 mmHg) to give the title compound as a colorless oil (40.5 g, 22%). ¹H NMR (500 MHz, CDCl₃) δ (ppm): 5.02 (t, J=5.7 Hz, 1H), 4.50 (d, J=5.7 Hz, 2H), 3.43 (s, 6H).

Step 2: To a solution of ortho amino heteroaromatic aldehyde 34 (1 equiv.) in THF (0.25 to 0.5 M) was added 1,1-dimethoxy-2-nitroethane 33 (3 equiv.) followed by p-TsOH (0.5 to 2 equiv.) in water equal to the volume of THF. The mixture was sealed and heated to 100° C. for 1 to 3 hours. The reaction was quenched with either saturated NaHCO₃ or Na₂CO₃ and extracted three times with either DCM or EtOAc. The combined organic layers were dried, filtered, and concentrated. The residue was either used directly in the next step or, purified by flash chromatography (SiO₂, eluting with EtOAc/hexane) as needed to afford the desired product 35.

### Example 20: General Procedure T

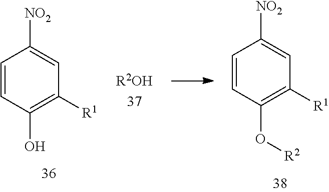

To a solution of triphenylphosphine (1.3 equiv.) in THF (0.06M) cooled to 0° C. was added a solution of 36 (1.1 equiv.). After the mixture was stirred at 0° C. for 10 min, alcohol 37 (1 equiv.) was added followed by diisopropyl-azodicarboxylate (1.3 equiv.). The mixture was allowed to warm to room temperature and was stirred for 48 h. The reaction was concentrated and reconstituted in ethyl acetate and extracted with 1 M HCl (3×). The combined organic phase was basified to pH 10 with 6 M NaOH and extracted with ethyl acetate (3×). The combined organic phase was dried, filtered, and concentrated under reduced pressure to give 38.

### Example 21: General Procedure U

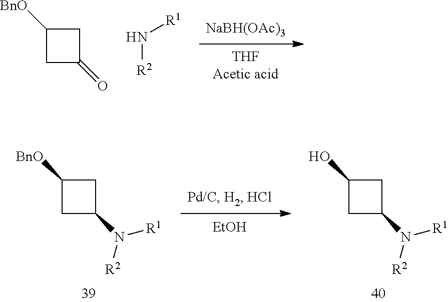

Step 1: To a solution of 3-(benzyloxy)cyclobutanone (1 equiv.) in THF and acetic acid (10:1, 0.20M) was added amine (1.1 equiv.). The solution was stirred for 1 h before sodium triacetoxyborohydride (2 equiv.) was added to the reaction mixture at 0° C., and the mixture was stirred at R.T. for 19 h. The mixture was then treated with sat. NaHCO₃ and concentrated under reduced pressure. The crude was extracted with EtOAc (3×). The combined organic layers were washed with brine, dried, filtered and concentrated under reduced pressure to give 39.

Step 2: To a solution of 39 (1 equiv.) and palladium on carbon (0.1 equiv.) in EtOH (0.20M) was added 4 M Hydrochloric acid in dioxane (4 equiv.) and the reaction was stirred at rt for 5 h under hydrogen atmosphere. The reaction was filtered on celite and washed three times with MeOH. The filtrate was concentrated to give the crude HCl salt. The crude salt was dissolved in a solution of CHCl₃/IPA (3:1) and basified with 6 M NaOH. The aqueous phase was extracted with CHCl₃/IPA (3:1) three times. The combined organic phase was dried, filtered, and concentrated under reduced pressure to give 40.

### Example 22: General Procedure V

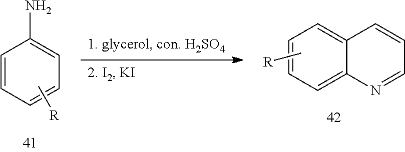

To a solution of sulfuric acid (5.0 equiv.) in glycerol (3.0 equiv.) preheated to 50° C. was charged aniline 41 (1.0 equiv.) and then heated to 90° C. for 40 min. Water (3.0 equiv.), iodine (0.05 equiv.), and potassium iodide (0.06 equiv.) were added and the reaction heated to 125° C. for 3 h. After cooling to room temperature, the reaction was filtered over celite topped and washed with ice-water. The filtrate was basified to pH 12 with 50% NaOH and extracted with Et₂O (3×). The combined organic layers were washed with water and brine then dried. The crude reaction was purified by flash column chromatography (SiO₂, eluting with EtOAc/hexanes) to afford the title product 42.

### Example 23: General Procedure W

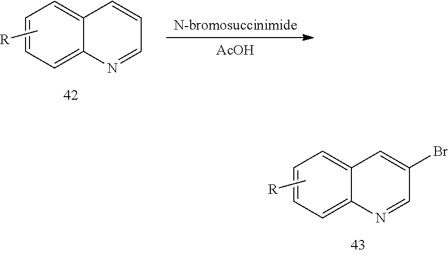

To a solution of aniline 42 (1.0 equiv.) in AcOH (0.5 M) at 110° C. oil bath for 10 min. To the reaction mixture was then added N-bromosuccinimide (1.1 equiv.) portionwise over 1 hour, followed by continued stirring for 3 h. AcOH was removed under reduced pressure and the remaining residue was partitioned between 1 M NaOH and CH₂Cl₂. The aqueous layer was further extracted with CH₂Cl₂ (2×) and the combined organic layers were washed with water and brine. The organic layer was dried, concentrated, and purified by chromatography (SiO₂, eluting with EtOAc/hexanes or C18, eluting with MeCN/0.1% of 10 mM ammonium formate buffer) to deliver the title product 43.

### Example 24: General Procedure X

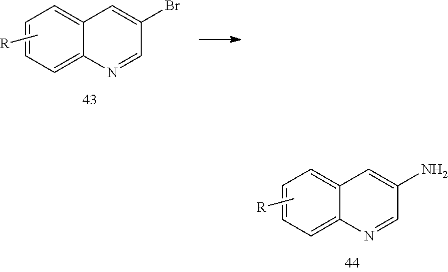

Step 1: To a degassed solution of 43 (1.0 equiv.), tert-Butyl carbamate (1.2 equiv.), and Xantphos (0.13 equiv.) in anhydrous dioxane (0.15 M) was added cesium carbonate (2.0 equiv.) and palladium(II) acetate (0.12 equiv.). The reaction was then heated to 100° C. for 16 h. The reaction was cooled, filtered, concentrated, and purified by flash column chromatography (SiO₂, MeOH/DCM) to afford the Boc-protected desired product.

Step 2: To the Boc-protected intermediate was added 4M HCl in dioxane (0.2 M) and reaction was stirred for 4 h. The resulting precipitate was collected to afford the desired HCl salt product 44 without further purification.

### Example 25: General Procedure Y

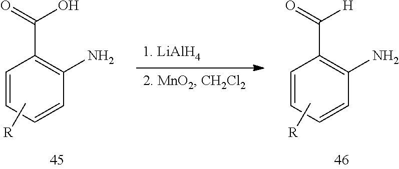

Step 1: Anthranilic acid 45 (1.0 equiv.) was dissolved in anhydrous THF (0.5 M) and cooled to 0° C. Lithium aluminum hydride (1.5 equiv.) (2 M solution in THF) was then added and the reaction stirred at room temperature for 2 h. The crude mixture was quenched at 0° C. with water, followed by 2M NaOH, and water. The slurry was then filtered through Celite and concentrated. The crude residue was redissolved in EtOAc and washed with water. The organic layer was then dried and concentrated to afford the intermediate benzyl alcohol.

Step 2: To the benzyl alcohol (1.0 equiv.) in DCM (0.25 M) was charged activated manganese (IV) oxide (8.0 equiv.). The reaction was stirred at room temperature for 16 h. The reaction mixture was filtered and concentrated under reduced pressure to deliver the title benzaldehyde 46.

### Example 26: General Procedure AD

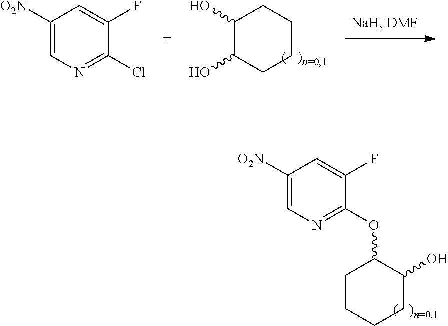

To a suspension of sodium hydride (60% in dispersion in mineral oil) (2 equiv.) in THF was added the corresponding diol xx (2 equiv.) and the reaction mixture was stirred for 20 min at rt. Then, 2-Chloro-3-fluoro-5-nitropyridine (1 equiv.) was added to the mixture and the reaction was stirred at 60° C. for 16 h. The reaction was then cooled to rt and quenched with NH₄Cl, extracted with EtOAc (3×) then washed with water and brine. The combined organic layers were dried, filtered, and concentrated. Purification by flash column chromatography (SiO₂, eluting with EtOAc/hexanes) afforded the desired compound in 82-95% yield.

### Example 27: General Procedure AE

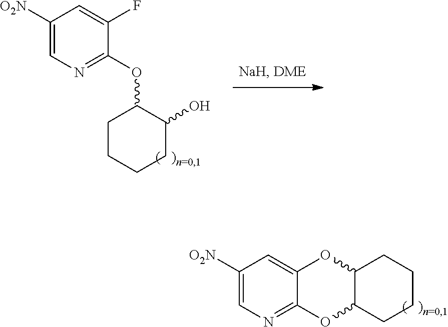

To a solution of the corresponding nitropyridine derivative (1 equiv.) in dimethoxyethane (0.2 M), was added sodium hydride (2 equiv.) in small portions at rt. The mixture was then heated at 80° C. for 12 h. The reaction was cooled, quenched with NH₄Cl, and extracted with EtOAc (3×). The combined organic layers were washed with water and brine, dried, filtered, and concentrated. Purification by flash column chromatography (SiO₂, eluting with EtOAc/hexanes) affords the desired product in 31-54% yields.

### Example 28: Preparation of 4-([1,4′-bipiperidin]-1′-yl)-3-methoxyaniline (Intermediate 1)

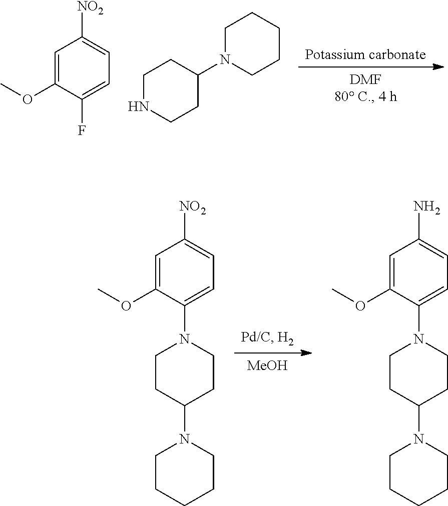

The title compound was prepared according to the SNAr general procedure G starting from 1-fluoro-2-methoxy-4-nitrobenzene and 4-piperidinopiperidine followed by nitro reduction employing the nitro general reduction procedure H. MS (m/z): [M+H]⁺: 290.2.

### Example 29: Preparation of Compound 1

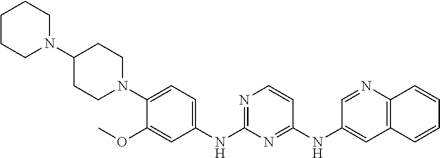 [SMILES: COc1cc(Nc2nccc(Nc3cnc4ccccc4c3)n2)ccc1N1CCC(N2CCCCC2)CC1]

Compound 1 was prepared from the corresponding intermediate according to general procedure E. MS (m/z): [M+H]⁺: 512.3. H NMR (500 MHz, DMSO) δ (ppm): 9.93 (s, 1H), 9.10 (s, 1H), 9.02 (s, 1H), 8.93 (s, 1H), 8.09 (d, J=5.6 Hz, 1H), 7.93 (d, J=8.3 Hz, 1H), 7.73 (s, 1H), 7.63-7.48 (m, 2H), 7.24 (d, J=10.1 Hz, 2H), 6.83 (d, J=8.4 Hz, 1H), 6.32 (d, J=5.5 Hz, 1H), 3.66 (s, 3H), 3.40 (d, J=10.7 Hz, 2H), 2.73-2.61 (m, 4H), 2.59-2.52 (m, 3H), 1.94-1.81 (m, 2H), 1.73-1.49 (m, 6H), 1.49-1.35 (m, 2H).

### Example 30: Preparation of 3-methoxy-4-(4-(4-methylpiperazin-1-yl)piperidin-1-yl)aniline (Intermediate 2)

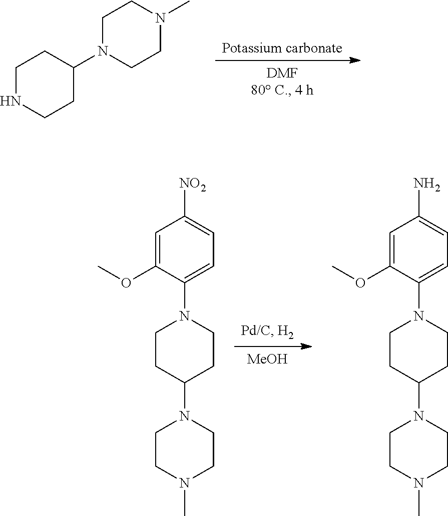

The title compound was prepared according to the SNAr general procedure G starting from 1-fluoro-2-methoxy-4-nitrobenzene and 1-methyl-4-(piperidin-4-yl)piperazine followed by nitro reduction employing the nitro general reduction procedure H. MS (m/z): [M+H]⁺: 305.4.

### Example 31: Preparation of Compound 2

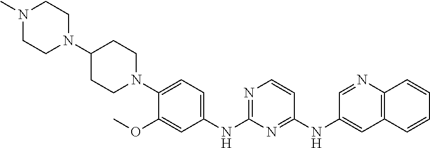 [SMILES: COc1cc(Nc2nccc(Nc3cnc4ccccc4c3)n2)ccc1N1CCC(N2CCN(C)CC2)CC1]

Compound 2 was prepared from the corresponding intermediate according to general procedure E. MS (m/z): [M+H]⁺: 525.9. ¹H NMR (500 MHz, DMSO) δ (ppm): 9.88 (s, 1H), 9.09 (s, 1H), 9.02 (s, 1H), 8.92 (s, 1H), 8.09 (d, J=5.6 Hz, 1H), 7.92 (t, J=11.8 Hz, 1H), 7.73 (s, 1H), 7.65-7.47 (m, 2H), 7.24 (d, J=8.5 Hz, 2H), 6.83 (d, J=8.2 Hz, 1H), 6.31 (d, J=5.6 Hz, 1H), 3.66 (s, 3H), 3.39 (d, J=11.4 Hz, 2H), 2.54 (s, 6H), 2.47-2.26 (m, 5H), 2.20 (s, 3H), 1.84 (d, J=11.3 Hz, 2H), 1.63-1.52 (m, 2H).

### Example 32: Preparation of 3-methoxy-4-(4-morpholinopiperidin-1-yl)aniline (Intermediate 3)

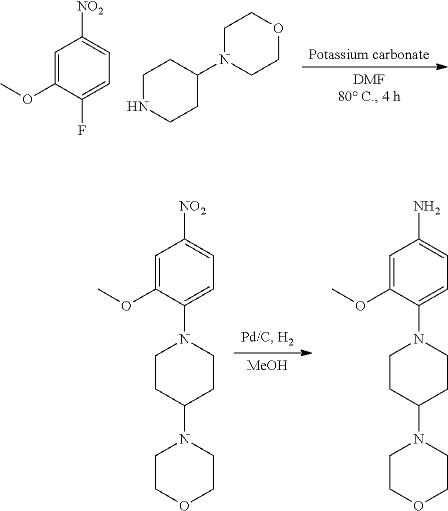

The title compound was prepared according to the SNAr general procedure G starting from 1-fluoro-2-methoxy-4-nitrobenzene and 4-morpholinopiperidine followed by nitro reduction employing the nitro general reduction procedure H. MS (m/z): [M+H]⁺: 292.2.

### Example 33: Preparation of Compound 3

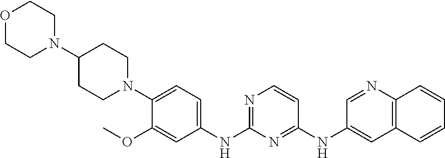 [SMILES: COc1cc(Nc2nccc(Nc3cnc4ccccc4c3)n2)ccc1N1CCC(N2CCOCC2)CC1]

Compound 3 was prepared from the corresponding intermediate according to general procedure E. MS (m/z): [M+H]⁺: 512.3. H NMR (500 MHz, DMSO) δ (ppm): 9.86 (s, 1H), 9.09 (s, 1H), 9.01 (s, 1H), 8.92 (d, J=2.3 Hz, 1H), 8.09 (d, J=5.7 Hz, 1H), 7.93 (d, J=8.4 Hz, 1H), 7.73 (s, 1H), 7.63-7.50 (m, 2H), 7.24 (d, J=9.3 Hz, 2H), 6.84 (d, J=8.3 Hz, 1H), 6.31 (d, J=5.7 Hz, 1H), 3.66 (s, 3H), 3.63-3.57 (m, 5H), 3.39 (d, J=11.4 Hz, 2H), 2.58-2.52 (m, 5H), 2.30-2.22 (m, 1H), 1.87 (s, 2H), 1.61-1.51 (m, 2H).

### Example 34: Preparation of 4-chloro-N-(3-methoxy-4-(3-(piperidin-1-yl)propoxy)phenyl)pyrimidin-2-amine (Intermediate 4)

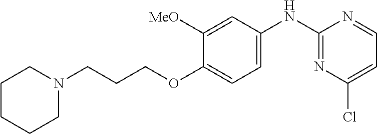 [SMILES: COc1cc(Nc2nccc(Cl)n2)ccc1OCCCN1CCCCC1]

### Step 1: Preparation of 1-(3-(2-methoxy-4-nitrophenoxy)propyl)piperidine

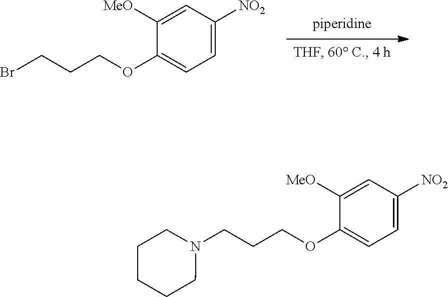 [SMILES: COc1cc([N+](=O)[O-])ccc1OCCCBr ; "THF, 60° C., 4 h" ; "piperidine" ; COc1cc([N+](=O)[O-])ccc1OCCCN1CCCCC1]

To a solution of 1-(3-bromopropoxy)-2-methoxy-4-nitrobenzene (1.00 g, 3.45 mmol) in THF (7 mL) was added piperidine (1.20 mL, 12.1 mmol). The mixture was stirred at 60° C. for 4 h. The reaction was cooled to rt and filtered. The filtrate was concentrated and redissolved in EtOAc (25 mL) and added to a K₂CO₃ 10% aq. solution (25 mL). The organic layer was extracted twice with EtOAc (5 mL). The combined organic layers were washed with brine (2×5 mL), dried, and concentrated to a yellow oil to afford the title product as a yellow oil (1.01 g, 100% yield). MS (m/z): [M+H]⁺: 295.3.

### Step 2: Preparation of 3-methoxy-4-(3-(piperidin-1-yl)propoxy)aniline

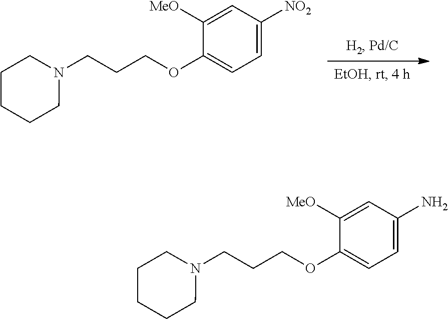 [SMILES: COc1cc([N+](=O)[O-])ccc1OCCCN1CCCCC1 ; "EtOH, rt, 4 h" ; "H2, Pd/C" ; COc1cc(N)ccc1OCCCN1CCCCC1]

The title compound was prepared following general procedure H, starting from 1-(3-(2-methoxy-4-nitrophenoxy)propyl)piperidine in EtOH. MS (m/z): [M+H]⁺: 265.3.

Step 3: Preparation of 4-chloro-N-(3-methoxy-4-(3-(piperidin-1-yl)propoxy)phenyl)-pyrimidin-2-amine

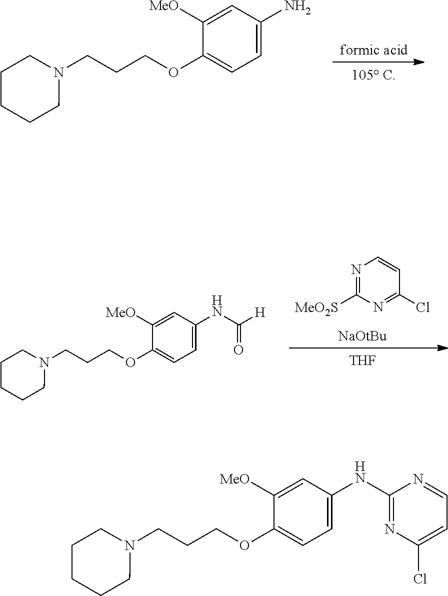

The title compound was prepared following general procedure A, starting from 3-methoxy-4-(3-(piperidin-1-yl)propoxy)aniline, 4-chloro-2-(methylsulfonyl)pyrimidine, and NaOtBu in THF. MS (m/z): [M+H]⁺: 377.5, 379.5.

### Example 35: Preparation of Compound 4

### Preparation of 10-methyl-6,7,8,9-tetrahydro-5H-5,8-epiminocyclohepta[b]pyridin-3-amine

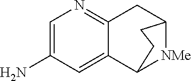 [SMILES: CN1C2CCC1c1cc(N)cnc1C2]

### Step 1: 10-methyl-3-nitro-6,7,8,9-tetrahydro-5H-5,8-epiminocyclohepta[b]pyridine

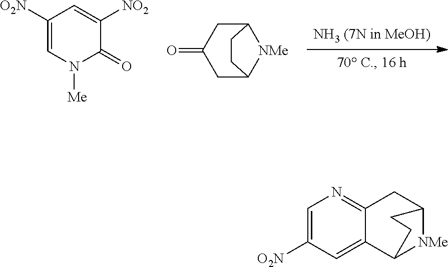 [SMILES: Cn1cc([N+](=O)[O-])cc([N+](=O)[O-])c1=O ; CN1C2CCC1CC(=O)C2 ; "70° C., 16 h" ; "NH3 (7N in MeOH)" ; CN1C2CCC1c1cc([N+](=O)[O-])cnc1C2]

The title compound was prepared following general procedure R starting from tropinone. MS (m/z): [M+H]⁺: 220.0.

### Step 2: 5,6,7,8-tetrahydro-5,8-ethanoquinolin-3-amine

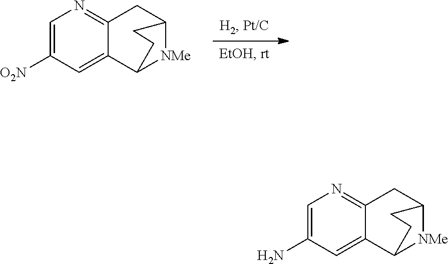 [SMILES: CN1C2CCC1c1cc([N+](=O)[O-])cnc1C2 ; "EtOH, rt" ; "H2, Pt/C" ; CN1C2CCC1c1cc(N)cnc1C2]

10-methyl-3-nitro-6,7,8,9-tetrahydro-5H-5,8-epiminocyclohepta[b]pyridine (125 mg, 0.57 mmol) was hydrogenated (1 atm) in the presence of Pt/C (5% on C) (67 mg) in EtOH (4 mL) for 2 h at rt. The mixture was diluted with EtOAc and filtered on Celite. The filtrate was concentrated to dryness to afford the title product (100 mg, 93% yield). MS (m/z): [M+H]⁺: 190.3.

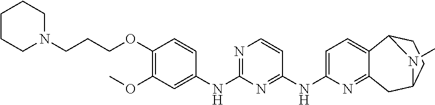 [SMILES: COc1cc(Nc2nccc(Nc3ccc4c(n3)CC3CCC4N3C)n2)ccc1OCCCN1CCCCC1]

Compound 4 was prepared from the corresponding intermediate according to general procedure C starting from 4-chloro-N-(3-methoxy-4-(3-(piperidin-1-yl)propoxy)phenyl)pyrimidin-2-amine and 10-methyl-6,7,8,9-tetrahydro-5H-5,8-epiminocyclohepta[b]pyridin-3-amine. MS (m/z): [M+H]⁺: 530.5. ¹H NMR (400 MHz, DMSO) δ (ppm): 9.36 (s, 1H), 8.94 (s, 1H), 8.45 (s, 1H), 8.00 (d, J=5.7 Hz, 1H), 7.94 (d, J=2.4 Hz, 1H), 7.25 (s, 1H), 7.17 (d, J=8.7 Hz, 1H), 6.85 (d, J=8.7 Hz, 1H), 6.17 (d, J=5.7 Hz, 1H), 3.93 (t, J=6.5 Hz, 2H), 3.72 (d, J=4.8 Hz, 1H), 3.66 (s, 3H), 3.43 (t, J=5.9 Hz, 1H), 3.09 (dd, J=17.3, 4.7 Hz, 1H), 2.45-2.29 (m, 7H), 2.26 (s, 3H), 2.22-2.03 (m, 2H), 1.88-1.78 (m, 2H), 1.62 (t, J=9.4 Hz, 1H), 1.54-1.45 (m, 5H), 1.42-1.35 (m, 2H).

### Example 36: Preparation of Compound 5

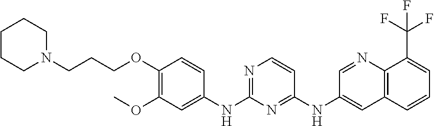 [SMILES: COc1cc(Nc2nccc(Nc3cnc4c(C(F)(F)F)cccc4c3)n2)ccc1OCCCN1CCCCC1]

Compound 5 was prepared from the corresponding intermediate according to general procedure C. MS (m/z): [M+H]⁺: 553.6. H NMR (500 MHz, DMSO) δ (ppm): 10.01 (s, 1H), 9.14 (s, 2H), 9.06 (s, 1H), 8.13 (d, J=5.7 Hz, 1H), 8.06-8.02 (m, 1H), 7.99 (d, J=7.2 Hz, 1H), 7.69 (d, J=7.7 Hz, 1H), 7.35-7.21 (m, 2H), 6.89 (d, J=8.7 Hz, 1H), 6.34 (d, J=5.7 Hz, 1H), 3.97 (t, J=6.5 Hz, 2H), 3.67 (s, 3H), 2.40 (t, J=7.2 Hz, 2H), 2.33 (s, 3H), 1.89-1.81 (m, 2H), 1.52-1.47 (m, 4H), 1.41-1.33 (m, 2H).

### Example 37: Preparation of Compound 6

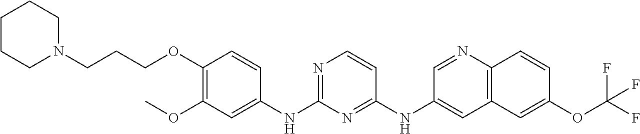 [SMILES: COc1cc(Nc2nccc(Nc3cnc4ccc(OC(F)(F)F)cc4c3)n2)ccc1OCCCN1CCCCC1]

Compound 6 was prepared from the corresponding intermediate according to general procedure C. MS (m/z): [M+H]⁺ 569.5. ¹H NMR (400 MHz, CD3OD) δ (ppm): 9.30 (s, 2H), 8.26 (d, J=9.3 Hz, 1H), 7.94 (s, 1H), 7.84 (d, J=8.5 Hz, 1H), 7.16-7.10 (m, 2H), 7.04 (dd, J=8.5, 2.2 Hz, 1H), 6.63 (d, J=7.2 Hz, 1H), 4.22 (t, J=5.6 Hz, 2H), 3.77 (s, 3H), 3.68 (d, J=12.0 Hz, 2H), 3.39 (t, J=7.5 Hz, 2H), 3.01 (t, J=12.4 Hz, 2H), 2.36-2.26 (m, 2H), 2.02-1.98 (m, 2H), 1.93-1.73 (m, 3H), 1.58 (dd, J=16.9, 8.2 Hz, 2H).

### Example 38: Preparation of 6,7-dihydro-5H-cyclopenta[b]pyridin-3-amine (Intermediate 5)

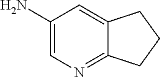

### Step 1: 3-nitro-6,7-dihydro-5H-cyclopenta[b]pyridine

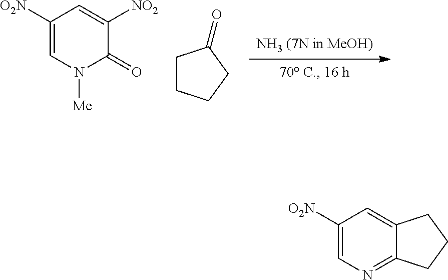

The title compound was prepared following general procedure R starting from cyclopentanone. ¹H NMR (500 MHz, CDCl3) δ (ppm): 9.14 (d, J=2.4 Hz, 1H), 8.19-8.17 (m, 1H), 3.05 (t, J=7.8 Hz, 2H), 3.00 (t, J=7.6 Hz, 2H), 2.20 (dd, J=15.3, 7.7 Hz, 2H).

### Step 2: 6,7-dihydro-5H-cyclopenta[b]pyridin-3-amine

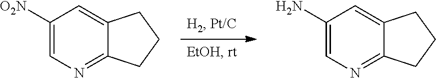 [SMILES: O=[N+]([O-])c1cnc2c(c1)CCC2 ; "EtOH, rt" ; "H2, Pt/C" ; Nc1cnc2c(c1)CCC2]

3-nitro-6,7-dihydro-5H-cyclopenta[b]pyridine (112 mg, 0.68 mmol) was hydrogenated (1 atm) in the presence of Pt/C (5% on C) (66 mg) in EtOH (5 mL) for 4 h at rt. The mixture was diluted with EtOAc and filtered on Celite. The filtrate was concentrated to dryness to afford the title product (79 mg, 86% yield). MS (m/z): [M+H]⁺: 135.

### Example 39: Preparation of 5,6,7,8-tetrahydro-5,8-ethanoquinolin-3-amine (Intermediate 6)

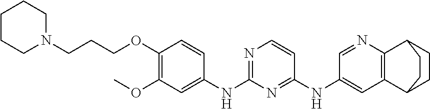 [SMILES: COc1cc(Nc2nccc(Nc3cnc4c(c3)C3CCC4CC3)n2)ccc1OCCCN1CCCCC1]

### Step 1: Preparation of 3-nitro-5,6,7,8-tetrahydro-5,8-ethanoquinoline

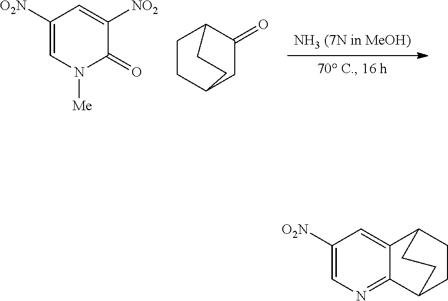

The title compound was prepared following general procedure R starting from bicyclo[2.2.2]octan-2-one. MS (m/z): [M+H]⁺: 205.1

### Step 2: Preparation of 5,6,7,8-tetrahydro-5,8-ethanoquinolin-3-amine

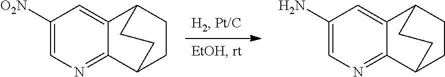 [SMILES: O=[N+]([O-])c1cnc2c(c1)C1CCC2CC1 ; "EtOH, rt" ; "H2, Pt/C" ; Nc1cnc2c(c1)C1CCC2CC1]

The title compound was prepared following general procedure H from 3-nitro-5,6,7,8-tetrahydro-5,8-ethanoquinoline. MS (m/z): [M+H]⁺: 175.3.

### Example 40: Preparation of Compound 209

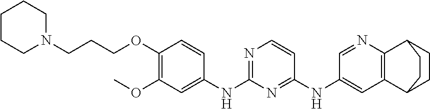 [SMILES: COc1cc(Nc2nccc(Nc3cnc4c(c3)C3CCC4CC3)n2)ccc1OCCCN1CCCCC1]

Compound 209 was prepared from the corresponding intermediate according to general procedure C. MS (m/z): [M+H]⁺ 515.6. H NMR (400 MHz, DMSO) δ (ppm): 9.29 (s, 1H), 8.88 (s, 1H), 8.32 (d, J=1.9 Hz, 1H), 8.07 (d, J=2.4 Hz, 1H), 7.94 (d, J=5.7 Hz, 1H), 7.15 (dd, J=8.6, 2.2 Hz, 1H), 6.77 (d, J=8.7 Hz, 1H), 6.12 (d, J=5.7 Hz, 1H), 3.87 (t, J=6.5 Hz, 2H), 3.60 (s, 3H), 2.97 (s, 1H), 2.93 (s, 1H), 2.35-2.20 (m, 6H), 1.80-1.68 (m, 6H), 1.47-1.42 (m, 4H), 1.36-1.26 (m, 6H).

### Example 41: Preparation of 4-chloro-N-(4-((1r,3r)-3-(dimethylamino)cyclobutoxy)-3-methoxyphenyl)pyrimidin-2-amine (Intermediate 7)

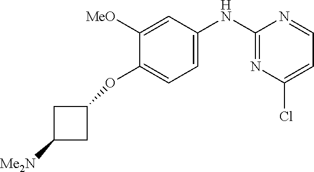 [SMILES: COc1cc(Nc2nccc(Cl)n2)ccc1O[C@H]1C[C@H](N(C)C)C1]

### Step 1: (1r,3r)-3-(2-methoxy-4-nitrophenoxy)-N,N-dimethylcyclobutanamine

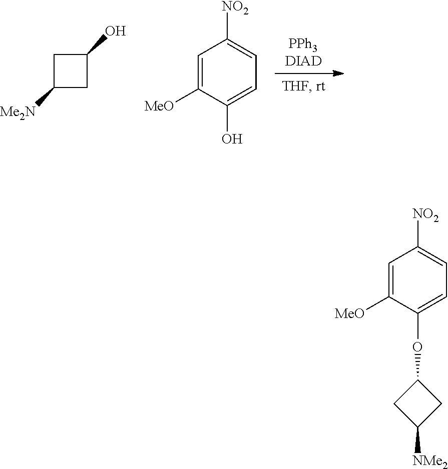 [SMILES: CN(C)[C@H]1C[C@@H](O)C1 ; COc1cc([N+](=O)[O-])ccc1O ; "THF, rt" ; COc1cc([N+](=O)[O-])ccc1O[C@H]1C[C@H](N(C)C)C1 ; "PPh3DIAD"]

The title compound was prepared following general procedure T from 4-nitroguaiacol. ¹H NMR (500 MHz, CDCl₃) δ (ppm): 7.86 (dd, J=8.9, 2.6 Hz, 1H), 7.75 (d, J=2.6 Hz, 1H), 6.68 (d, J=8.9 Hz, 1H), 4.97-4.91 (m, 1H), 3.95 (s, 3H), 3.15 (brs, 1H), 2.66 (brs, 2H), 2.53-2.41 (m, 2H), 2.32 (s, 6H). MS (m/z): [M+H]⁺: 267.

### Step 2: 4-((1r,3r)-3-(dimethylamino)cyclobutoxy)-3-methoxyaniline

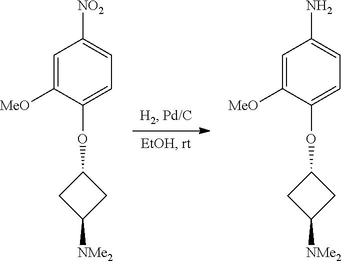 [SMILES: COc1cc([N+](=O)[O-])ccc1O[C@H]1C[C@H](N(C)C)C1 ; "EtOH, rt" ; "H2, Pd/C" ; COc1cc(N)ccc1O[C@H]1C[C@H](N(C)C)C1]

The title compound was prepared following general procedure H, starting from (1r,3r)-3-(2-methoxy-4-nitrophenoxy)-N,N-dimethylcyclobutanamine in EtOH. MS (m/z): [M+H]⁺: 237.3.

### Step 3: 4-chloro-N-(4-((1r,3r)-3-(dimethylamino)cyclobutoxy)-3-methoxyphenyl)-pyrimidin-2-amine

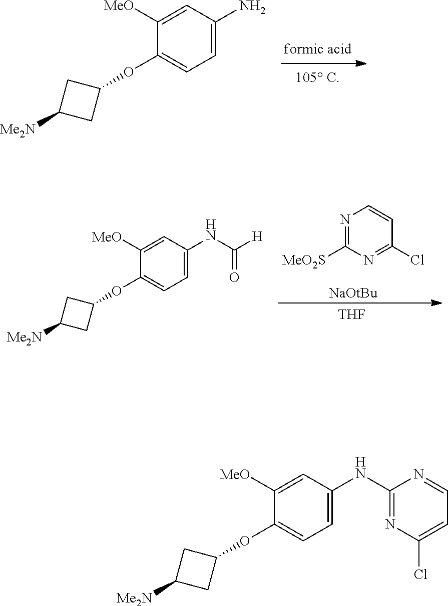 [SMILES: COc1cc(N)ccc1O[C@H]1C[C@H](N(C)C)C1 ; COc1cc(NC=O)ccc1O[C@H]1C[C@H](N(C)C)C1 ; CS(=O)(=O)c1nccc(Cl)n1 ; COc1cc(Nc2nccc(Cl)n2)ccc1O[C@H]1C[C@H](N(C)C)C1 ; "NaOtBu" ; "THF"]

The title compound was prepared following general procedure A, starting from 4-((1r,3r)-3-(dimethylamino)cyclobutoxy)-3-methoxyaniline, 4-chloro-2-(methylsulfonyl)pyrimidine, and NaOtBu in THF. MS (m/z): [M+H]⁺: 349.5, 351.4.

### Example 42: Preparation of Compound 7

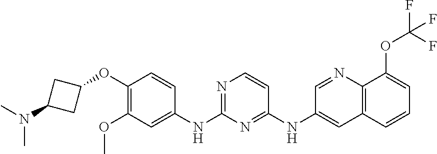 [SMILES: COc1cc(Nc2nccc(Nc3cnc4c(OC(F)(F)F)cccc4c3)n2)ccc1O[C@H]1C[C@H](N(C)C)C1]

Compound 7 was prepared from the corresponding intermediate according to general procedure C. MS (m/z): [M+H]⁺ 541.3. ¹H NMR (400 MHz, DMSO) δ (ppm): 10.00 (s, 1H), 9.13 (s, 1H), 9.10 (s, 1H), 9.05 (s, 1H), 8.12 (d, J=5.7 Hz, 1H), 7.78 (s, 1H), 7.61 (d, J=5.5 Hz, 2H), 7.30-7.19 (m, 2H), 6.69 (d, J=8.7 Hz, 1H), 6.33 (d, J=5.7 Hz, 1H), 4.71-4.64 (m, 1H), 3.67 (s, 3H), 2.82 (td, J=7.6, 3.8 Hz, 1H), 2.34-2.26 (m, 2H), 2.20-2.11 (m, 2H), 2.08 (s, 6H).

### Example 43: Preparation of Compound 8

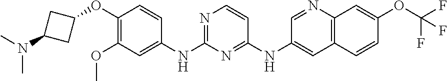 [SMILES: COc1cc(Nc2nccc(Nc3cnc4cc(OC(F)(F)F)ccc4c3)n2)ccc1O[C@H]1C[C@H](N(C)C)C1]

Compound 8 was prepared from the corresponding intermediate according to general procedure C. MS (m/z): [M+H]⁺ 541.3. H NMR (500 MHz, DMSO) δ (ppm): 9.93 (s, 1H), 9.13-8.97 (m, 3H), 8.11 (d, J=5.7 Hz, 1H), 7.91-7.85 (m, 2H), 7.58 (d, J=8.8 Hz, 1H), 7.29-7.19 (m, 2H), 6.69 (d, J=8.7 Hz, 1H), 6.31 (d, J=5.7 Hz, 1H), 4.70-4.64 (m, 1H), 3.66 (s, 3H), 2.85-2.80 (m, 1H), 2.34-2.24 (m, 2H), 2.21-2.12 (m, 2H), 2.08 (s, 6H).

### Example 44: Preparation of 4-chloro-N-(4-((1s,3s)-3-(dimethylamino)cyclobutoxy)-3-methoxyphenyl)pyrimidin-2-amine (Intermediate 8)

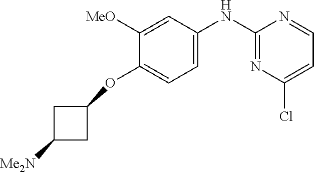 [SMILES: COc1cc(Nc2nccc(Cl)n2)ccc1O[C@H]1C[C@@H](N(C)C)C1]

### Step 1: (1s,3s)-3-(2-methoxy-4-nitrophenoxy)-N,N-dimethylcyclobutanamine

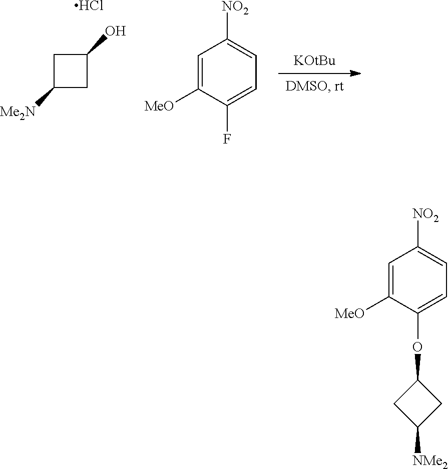 [SMILES: Cl ; CN(C)[C@H]1C[C@@H](O)C1 ; COc1cc([N+](=O)[O-])ccc1F ; "DMSO, rt" ; COc1cc([N+](=O)[O-])ccc1O[C@H]1C[C@@H](N(C)C)C1 ; "KOtBu"]

The title compound was prepared following general procedure G from (1s,3s)-3-(dimethylamino)cyclobutanol hydrochloride and 1-fluoro-2-methoxy-4-nitrobenzene. ¹H NMR (400 MHz, CDCl₃): δ (ppm): 7.86 (dd, J=8.9, 2.6 Hz, 1H), 7.73 (d, J=2.5 Hz, 1H), 6.76 (d, J=8.9 Hz, 1H), 4.50 (p, J=7.2 Hz, 1H), 3.93 (s, 3H), 2.78-2.67 (m, 2H), 2.49-2.39 (m, 1H), 2.20-2.08 (m, 8H). MS (m/z): [M+H]⁺: 267.2.

### Step 2: 4-((1s,3s)-3-(dimethylamino)cyclobutoxy)-3-methoxyaniline

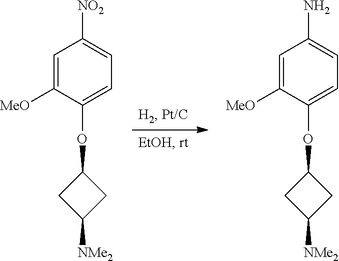 [SMILES: COc1cc([N+](=O)[O-])ccc1O[C@H]1C[C@@H](N(C)C)C1 ; "EtOH, rt" ; "H2, Pt/C" ; COc1cc(N)ccc1O[C@H]1C[C@@H](N(C)C)C1]

The title compound was prepared following general procedure H, starting from (1s,3s)-3-(2-methoxy-4-nitrophenoxy)-N,N-dimethylcyclobutanamine in EtOH. MS (m/z): [M+H]⁺: 237.2.

### Step 3: 4-chloro-N-(4-((1s,3s)-3-(dimethylamino)cyclobutoxy)-3-methoxyphenyl) pyrimidin-2-amine

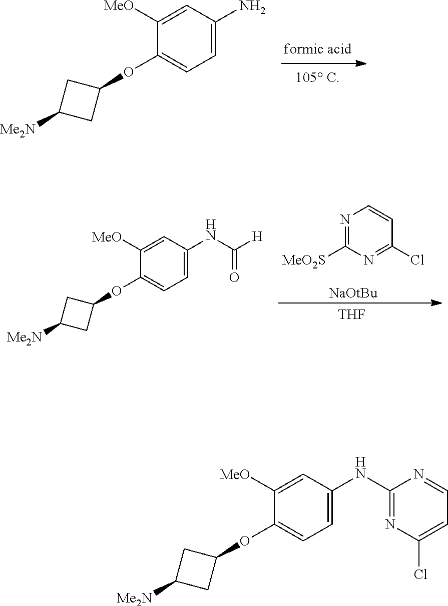 [SMILES: COc1cc(N)ccc1O[C@H]1C[C@@H](N(C)C)C1 ; COc1cc(NC=O)ccc1O[C@H]1C[C@@H](N(C)C)C1 ; CS(=O)(=O)c1nccc(Cl)n1 ; COc1cc(Nc2nccc(Cl)n2)ccc1O[C@H]1C[C@@H](N(C)C)C1 ; "NaOtBu" ; "THF"]

The title compound was prepared following general procedure A, starting from 4-((1s,3s)-3-(dimethylamino)cyclobutoxy)-3-methoxyaniline, 4-chloro-2-(methylsulfonyl)pyrimidine, and NaOtBu in THF. MS (m/z): [M+H]⁺: 349.5, 351.4.

### Example 45: Preparation of Compound 9

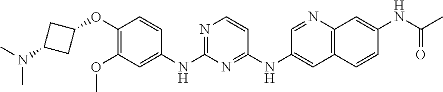 [SMILES: COc1cc(Nc2nccc(Nc3cnc4cc(NC(C)=O)ccc4c3)n2)ccc1O[C@H]1C[C@@H](N(C)C)C1]

Compound 9 was prepared from the corresponding intermediate according to general procedure D. MS (m/z): [M+H]⁺ 514.7. H NMR (500 MHz, DMSO) δ (ppm): 10.22 (s, 1H), 9.74 (s, 1H), 9.05 (s, 1H), 8.88 (s, 1H), 8.84 (s, 1H), 8.31 (s, 1H), 8.07 (d, J=5.2 Hz, 1H), 7.69 (s, 2H), 7.28 (s, 1H), 7.24 (s, 1H), 6.75 (d, J=8.5 Hz, 1H), 6.28 (d, J=5.1 Hz, 1H), 4.38-4.32 (m, 1H), 3.64 (s, 3H), 2.57 (s, 2H), 2.13 (s, 3H), 2.07 (s, 6H), 1.90-1.80 (m, 2H).

### Example 46: Preparation of Compound 10

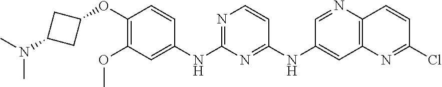 [SMILES: COc1cc(Nc2nccc(Nc3cnc4ccc(Cl)nc4c3)n2)ccc1O[C@H]1C[C@@H](N(C)C)C1]

Compound 10 was prepared from the corresponding intermediate according to general procedure C. MS (m/z): [M+H]⁺ 492.5, 494.6. H NMR (400 MHz, dmso) δ (ppm): 10.08 (s, 1H), 9.27 (s, 1H), 9.09 (d, J=2.5 Hz, 1H), 8.99 (d, J=1.9 Hz, 1H), 8.35 (dd, J=8.7, 0.7 Hz, 1H), 8.14 (d, J=5.6 Hz, 1H), 7.64 (d, J=8.7 Hz, 1H), 7.35 (dd, J=8.7, 2.4 Hz, 1H), 7.29 (s, 1H), 6.78 (d, J=8.7 Hz, 1H), 6.35 (d, J=5.7 Hz, 1H), 4.36 (p, J=7.2 Hz, 1H), 3.69 (s, 3H), 2.60-2.52 (m, 2H), 2.34-2.25 (m, 1H), 1.85-1.78 (m, 2H).

### Example 47: Preparation of 4-chloro-N-(6-((1s,3s)-3-(dimethylamino)cyclobutoxy)-5-methoxypyridin-3-yl)pyrimidin-2-amine (Intermediate 9)

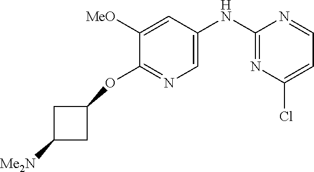 [SMILES: COc1cc(Nc2nccc(Cl)n2)cnc1O[C@H]1C[C@@H](N(C)C)C1]

### Step 1: (1s,3s)-3-((3-methoxy-5-nitropyridin-2-yl)oxy)-N,N-dimethylcyclobutanamine

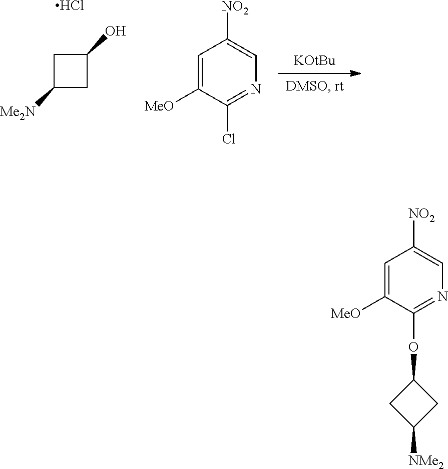 [SMILES: Cl ; CN(C)[C@H]1C[C@@H](O)C1 ; COc1cc([N+](=O)[O-])cnc1Cl ; "DMSO, rt" ; COc1cc([N+](=O)[O-])cnc1O[C@H]1C[C@@H](N(C)C)C1 ; "KOtBu"]

The title compound was prepared following general procedure G starting from (1s,3s)-3-(dimethylamino)cyclobutanol hydrochloride and 2-chloro-3-methoxy-5-nitropyridine. MS (m/z): [M+H]⁺: 268.

### Step 2: 6-((1s,3s)-3-(dimethylamino)cyclobutoxy)-5-methoxypyridin-3-amine

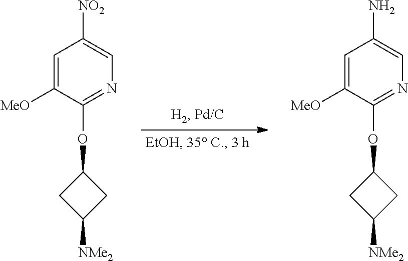 [SMILES: COc1cc([N+](=O)[O-])cnc1O[C@H]1C[C@@H](N(C)C)C1 ; "EtOH, 35° C., 3 h" ; "H2, Pd/C" ; COc1cc(N)cnc1O[C@H]1C[C@@H](N(C)C)C1]

The title compound was prepared following general procedure H, starting from (1s,3s)-3-((3-methoxy-5-nitropyridin-2-yl)oxy)-N,N-dimethylcyclobutanamine in EtOH at 35° C. MS (m/z): [M+H]⁺: 238.2.

### Step 3: 4-chloro-N-(6-((1s,3s)-3-(dimethylamino)cyclobutoxy)-5-methoxypyridin-3-yl)pyrimidin-2-amine

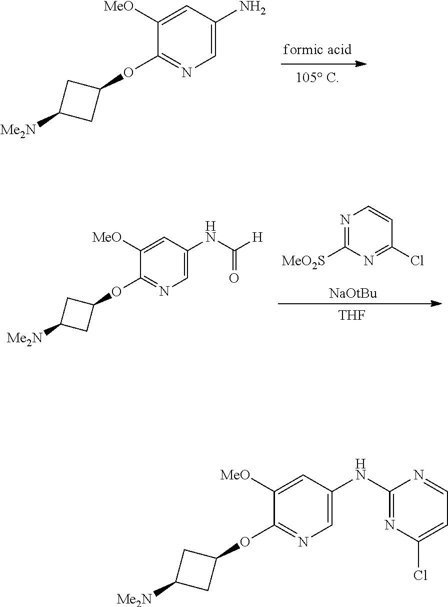 [SMILES: COc1cc(N)cnc1O[C@H]1C[C@@H](N(C)C)C1 ; COc1cc(NC=O)cnc1O[C@H]1C[C@@H](N(C)C)C1 ; CS(=O)(=O)c1nccc(Cl)n1 ; COc1cc(Nc2nccc(Cl)n2)cnc1O[C@H]1C[C@@H](N(C)C)C1 ; "NaOtBu" ; "THF"]

The title compound was prepared following general procedure A, starting from 6-((1s,3s)-3-(dimethylamino)cyclobutoxy)-5-methoxypyridin-3-amine, 4-chloro-2-(methylsulfonyl)pyrimidine, and NaOtBu in THF. MS (m/z): [M+H]⁺: 350.2, 352.4.

### Example 48: Preparation of Compound 11

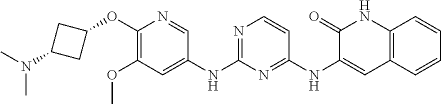 [SMILES: COc1cc(Nc2nccc(Nc3cc4ccccc4[nH]c3=O)n2)cnc1O[C@H]1C[C@@H](N(C)C)C1]

Compound 11 was prepared from the corresponding intermediate according to general procedure C. MS (m/z): [M+H]⁺ 474.4. H NMR (500 MHz, DMSO) δ 12.23 (s, 1H), 9.14 (s, 1H), 9.01 (s, 1H), 8.89 (s, 1H), 8.07 (d, J=5.7 Hz, 1H), 8.05 (d, J=2.1 Hz, 1H), 7.54 (s, 1H), 7.46 (s, 1H), 7.40-7.34 (m, 1H), 7.29 (d, J=8.1 Hz, 1H), 7.17 (t, J=7.2 Hz, 1H), 6.76 (d, J=5.8 Hz, 1H), 4.89 (p, J=7.5 Hz, 1H), 3.72 (s, 3H), 2.57-2.65 (m, 2H), 2.42-2.32 (m, 1H), 2.07 (s, 6H), 1.90-1.83 (m, 2H).

### Example 49: Preparation of Compound 12

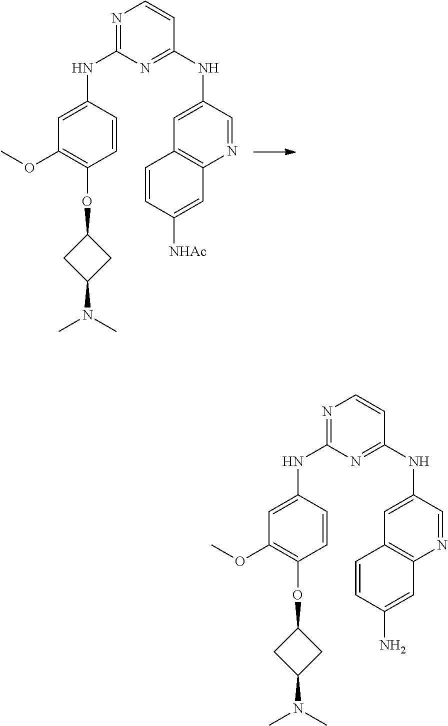 [SMILES: COc1cc(Nc2nccc(Nc3cnc4cc(NC(C)=O)ccc4c3)n2)ccc1O[C@H]1C[C@@H](N(C)C)C1 ; COc1cc(Nc2nccc(Nc3cnc4cc(N)ccc4c3)n2)ccc1O[C@H]1C[C@@H](N(C)C)C1]

To a solution of Compound 9 (42.0 mg, 0.078 mmol) in dioxane (1.0 mL) was added 6M HCl (1.0 mL) and the mixture was heated to 90° C. for 1 h. After cooling to rt, the reaction was neutralized with 6M NaOH, concentrated to dryness, and purified by flash chromatography (CH₂Cl₂/MeOH/NH₄OH) to afford the title product as a white solid (20.0 mg, 54% yield). MS (m/z): [M+H]⁺ 472.5. ¹H NMR (500 MHz, DMSO) δ (ppm): 9.45 (s, 1H), 8.95 (s, 1H), 8.68 (d, J=2.4 Hz, 1H), 8.57 (s, 1H), 8.00 (d, J=5.7 Hz, 1H), 7.46 (d, J=8.5 Hz, 1H), 7.28 (s, 1H), 7.22 (d, J=8.6 Hz, 1H), 6.97 (dd, J=8.7, 2.2 Hz, 1H), 6.93 (d, J=2.1 Hz, 1H), 6.70 (d, J=8.7 Hz, 1H), 6.20 (d, J=5.7 Hz, 1H), 5.56 (s, 1H), 4.32 (p, J=7.3 Hz, 1H), 3.60 (s, 3H), 2.60-2.52 (m, 2H), 2.38-2.32 (m, 1H), 2.08 (s, 6H), 1.87-1.78 (m, 2H).

### Example 50: Preparation of 3-methoxy-4-(2-(piperidin-1-yl)ethoxy)aniline (Intermediate 10)

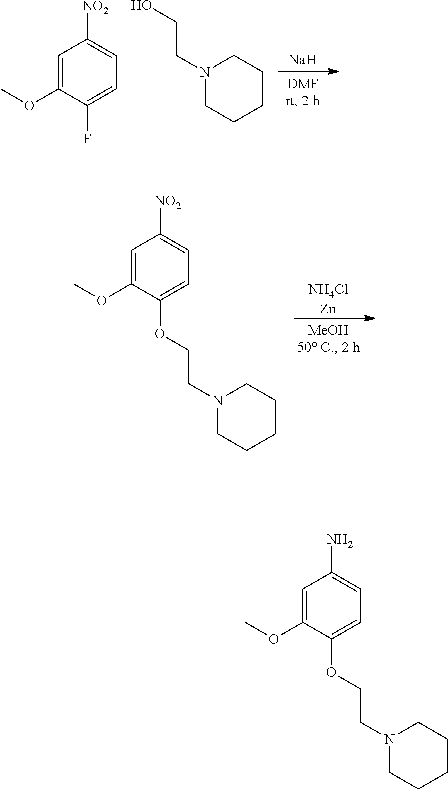

The title compound was prepared following general procedure G, starting from 1-fluoro-2-methoxy-4-nitrobenzene in DMF followed by nitro reduction employing the general nitro reduction procedure I. MS (m/z): [M+H]⁺: 250.9.

### Example 51: Preparation of Compound 13

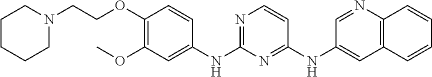 [SMILES: COc1cc(Nc2nccc(Nc3cnc4ccccc4c3)n2)ccc1OCCN1CCCCC1]

Compound 13 was prepared from the corresponding intermediate according to general procedure E. MS (m/z): [M+H]⁺: 471.4. H NMR (500 MHz, DMSO): δ (ppm): 9.81 (s, 1H), 9.07 (s, 1H), 8.95 (s, 1H), 8.93 (d, J=2.4 Hz, 1H), 8.08 (d, J=5.7 Hz, 1H), 7.92 (d, J=8.3 Hz, 1H), 7.75 (s, 1H), 7.62-7.56 (m, 1H), 7.56-7.50 (m, 1H), 7.30 (s, 1H), 7.23 (d, J=8.2 Hz, 1H), 6.90 (d, J=8.7 Hz, 1H), 6.30 (d, J=5.7 Hz, 1H), 4.03 (t, J=6.1 Hz, 2H), 3.64 (s, 3H), 2.65 (t, J=6.1 Hz, 2H), 2.44 (s, 4H), 1.51 (dt, J=11.1, 5.5 Hz, 4H), 1.44-1.35 (m, 2H).

### Example 52: Preparation of 3-methoxy-4-(3-morpholinopropoxy)aniline (Intermediate 11)

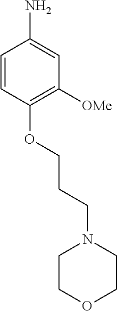 [SMILES: COc1cc(N)ccc1OCCCN1CCOCC1]

### Step 1: 4-(3-(2-methoxy-4-nitrophenoxy)propyl)morpholine

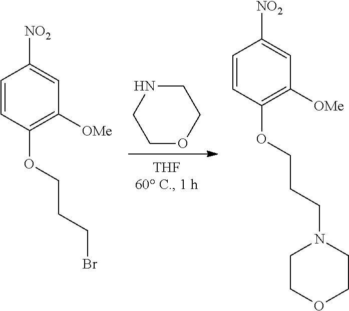

To a solution of 1-(3-bromopropoxy)-2-methoxy-4-nitrobenzene (150 mg, 517 umol) in THF (1.0 mL) was added morpholine (159 mL, 1.82 mmol). The mixture was stirred at 60° C. for 1 h. The reaction was cooled to rt and the precipitate was removed by filtration. The filtrate was concentrated to a yellow oil and re-dissolved in EtAOc and water. The aqueous layer was extracted with EtOAc three times. The combined organic layers were washed with brine twice and concentrated to give 4-(3-(2-methoxy-4-nitrophenoxy)propyl)morpholine (150 mg, 98%) as a pale yellow solid.

### Step 2: 3-methoxy-4-(3-morpholinopropoxy)aniline

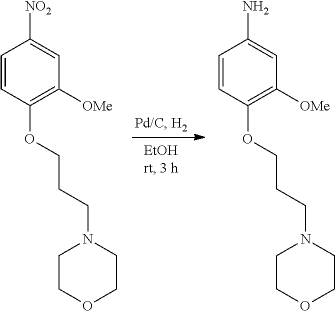 [SMILES: COc1cc([N+](=O)[O-])ccc1OCCCN1CCOCC1 ; COc1cc(N)ccc1OCCCN1CCOCC1]

The title compound was prepared following general procedure H starting from 4-(3-(2-methoxy-4-nitrophenoxy)propyl)morpholine in EtOH. MS (m/z): [M+H]⁺: 266.9.

### Example 53: Preparation of Compound 14

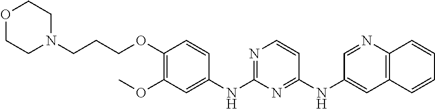 [SMILES: COc1cc(Nc2nccc(Nc3cnc4ccccc4c3)n2)ccc1OCCCN1CCOCC1]

Compound 14 was prepared from the corresponding intermediate according to general procedure E. MS (m/z): [M+H]⁺ 487.4. H NMR (500 MHz, DMSO): δ (ppm): 9.82 (s, 1H), 9.08 (s, 1H), 8.96 (s, 1H), 8.93 (d, J=2.3 Hz, 1H), 8.08 (d, J=5.7 Hz, 1H), 7.92 (d, J=8.3 Hz, 1H), 7.74 (s, 1H), 7.62-7.56 (m, 1H), 7.54 (t, J=6.8 Hz, 1H), 7.29 (s, 1H), 7.23 (d, J=8.6 Hz, 1H), 6.88 (d, J=8.7 Hz, 1H), 6.30 (d, J=5.7 Hz, 1H), 3.98 (t, J=6.4 Hz, 2H), 3.64 (s, 3H), 3.57 (t, J=4.6 Hz, 4H), 2.44 (t, J=7.2 Hz, 2H), 2.37 (s, 4H), 1.87 (p, J=6.7 Hz, 2H).

### Example 54: Preparation of Compound 15

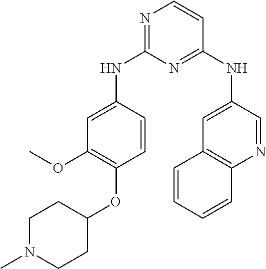 [SMILES: COc1cc(Nc2nccc(Nc3cnc4ccccc4c3)n2)ccc1OC1CCN(C)CC1]

### Step 1: 2-methoxy-4-((4-(quinolin-3-ylamino)pyrimidin-2-yl)amino)phenol

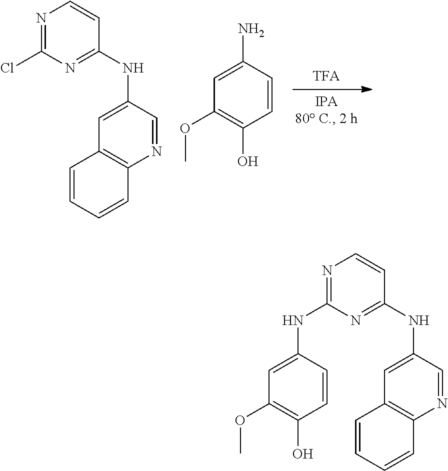

The title compound was prepared following general procedure E, starting from N-(2-chloropyrimidin-4-yl)quinolin-3-amine and 4-amino-2-methoxyphenol in IPA. MS (m/z): [M+H]⁺: 360.4.

### Step 2

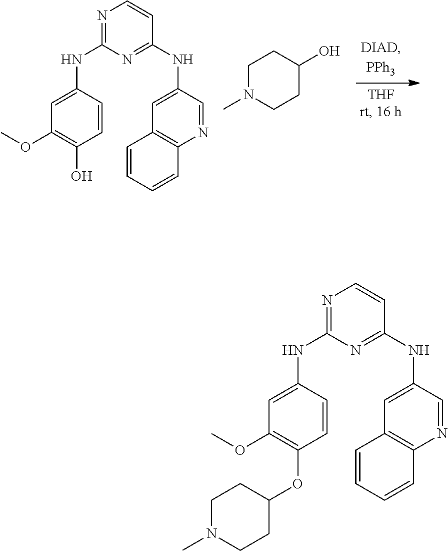

The title compounds was prepared following general procedure T starting from 2-methoxy-4-((4-(quinolin-3-ylamino)pyrimidin-2-yl)amino)phenol and 4-hydroxy-1-methylpiperidine. MS (m/z): [M+H]⁺: 457.5. ¹H NMR (500 MHz, DMSO): δ (ppm): 9.85 (s, 1H), 9.15 (s, 1H), 8.97 (s, 1H), 8.95 (d, J=2.4 Hz, 1H), 8.15 (s, 1H), 8.10 (d, J=5.7 Hz, 1H), 7.94 (d, J=8.3 Hz, 1H), 7.76 (s, 1H), 7.60 (ddd, J=8.4, 6.9, 1.5 Hz, 1H), 7.54 (t, J=6.9 Hz, 1H), 7.35 (s, 1H), 7.24 (d, J=7.0 Hz, 1H), 6.93 (d, J=8.7 Hz, 1H), 6.32 (d, J=5.7 Hz, 1H), 4.30-4.16 (m, 1H), 3.64 (s, 3H), 2.95-2.82 (m, 2H), 2.40 (s, 3H), 2.03-1.92 (m, 2H), 1.81-1.65 (m, 2H), two protons are overlapped with water peak.

### Example 55: Preparation of 3-methoxy-4-(((1r,4r)-4-morpholinocyclohexyl)oxy)aniline (Intermediate 12)

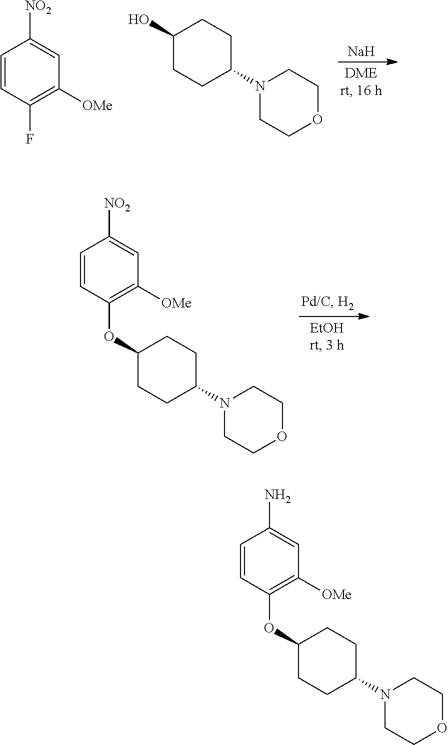

The title compound was prepared following general procedure G, starting from 1-fluoro-2-methoxy-4-nitrobenzene and (1r,4r)-4-morpholinocyclohexan-1-ol in DME followed by nitro reduction employing the general nitro reduction procedure H.

### Example 56: Preparation of Compound 16

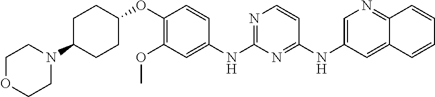 [SMILES: COc1cc(Nc2nccc(Nc3cnc4ccccc4c3)n2)ccc1O[C@H]1CC[C@H](N2CCOCC2)CC1]

Compound 16 was prepared from the corresponding intermediate according to general procedure E. MS (m/z): [M+H]⁺ 527.5. H NMR (500 MHz, DMSO) δ (ppm): 9.83 (s, 1H), 9.10 (s, 1H), 8.97 (s, 1H), 8.94 (d, J=2.5 Hz, 1H), 8.09 (d, J=5.7 Hz, 1H), 7.94 (d, J=8.3 Hz, 1H), 7.76 (d, J=6.7 Hz, 1H), 7.60 (ddd, J=8.3, 6.9, 1.5 Hz, 1H), 7.53 (t, J=7.2 Hz, 1H), 7.31 (s, 1H), 7.23 (d, J=7.9 Hz, 1H), 6.89 (d, J=8.5 Hz, 1H), 6.31 (d, J=5.7 Hz, 1H), 4.11-4.00 (m, 1H), 3.63 (s, 3H), 3.61-3.49 (m, 4H), 2.50-2.39 (m, 4H), 2.32-2.19 (m, 1H), 2.16-2.02 (m, 2H), 1.96-1.78 (m, 2H), 1.49-1.25 (m, 4H).

### Example 57: Preparation of 4-(((1r,4r)-4-(dimethylamino)cyclohexyl)oxy)-3-methoxyaniline (Intermediate 13)

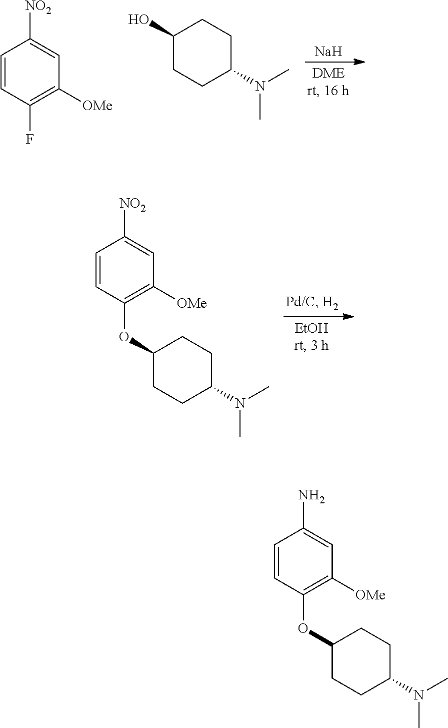

The title compound was prepared following general procedure G, starting from 1-fluoro-2-methoxy-4-nitrobenzene and (1r,4r)-4-(dimethylamino)cyclohexan-1-ol in DME followed by nitro reduction employing the general nitro reduction procedure H. MS (m/z): [M+H]⁺: 265.3.

### Example 58: Preparation of Compound 17

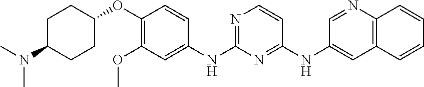 [SMILES: COc1cc(Nc2nccc(Nc3cnc4ccccc4c3)n2)ccc1O[C@H]1CC[C@H](N(C)C)CC1]

Compound 17 was prepared from the corresponding intermediate according to general procedure E. MS (m/z): [M+H]⁺ 485.5. ¹H NMR (500 MHz, DMSO) δ (ppm): 9.82 (s, 1H), 9.09 (s, 1H), 8.97 (s, 1H), 8.93 (d, J=2.5 Hz, 1H), 8.08 (d, J=5.7 Hz, 1H), 7.93 (d, J=8.1 Hz, 1H), 7.79-7.72 (m, 1H), 7.59 (ddd, J=8.4, 6.9, 1.5 Hz, 1H), 7.52 (t, J=7.5 Hz, 1H), 7.30 (s, 1H), 7.22 (d, J=8.4 Hz, 1H), 6.88 (d, J=8.7 Hz, 1H), 6.30 (d, J=5.7 Hz, 1H), 4.10-4.00 (m, 1H), 3.62 (s, 3H), 2.25-2.14 (m, 7H), 2.11-2.01 (m, 2H), 1.87-1.77 (m, 2H), 1.45-1.34 (m, 2H), 1.33-1.23 (m, 2H).

### Example 59: Preparation of 3-methoxy-4-(((1s,4s)-4-morpholinocyclohexyl)oxy)aniline (Intermediate 14)

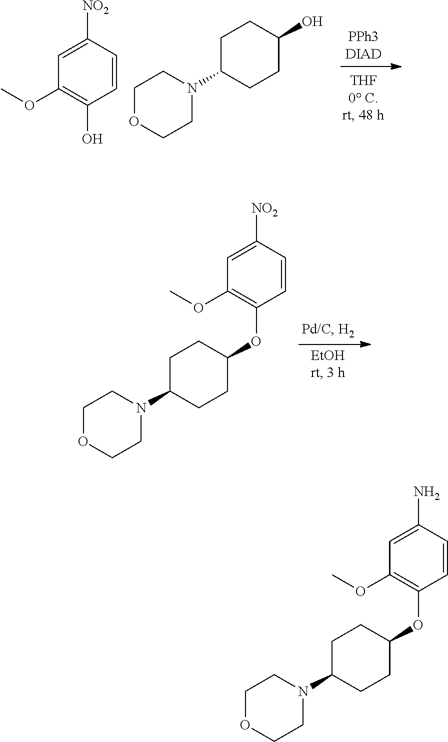

The title compound was prepared following general procedure T, starting from 2-methoxy-4-nitrophenol and (1r,4r)-4-morpholinocyclohexan-1-ol in THF followed by nitro reduction employing the general nitro reduction procedure H. MS (m/z): [M+H]⁺: 306.9.

### Example 60: Preparation of Compound 18

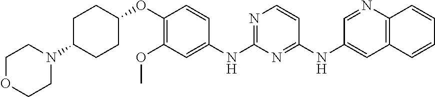 [SMILES: COc1cc(Nc2nccc(Nc3cnc4ccccc4c3)n2)ccc1O[C@H]1CC[C@@H](N2CCOCC2)CC1]

Compound 18 was prepared from the corresponding intermediate according to general procedure E. MS (m/z): [M+H]⁺ 527.5. H NMR (500 MHz, DMSO) δ (ppm): 9.83 (s, 1H), 9.12 (s, 1H), 8.98 (s, 1H), 8.94 (d, J=2.4 Hz, 1H), 8.10 (d, J=5.7 Hz, 1H), 7.93 (d, J=8.4 Hz, 1H), 7.75 (s, 1H), 7.67-7.59 (m, 2H), 7.33 (s, 1H), 7.23 (d, J=8.3 Hz, 1H), 6.90 (d, J=8.7 Hz, 1H), 6.31 (d, J=5.7 Hz, 1H), 4.36 (dd, J=5.8, 1.6 Hz, 1H), 3.64 (s, 3H), 3.62-3.56 (m, 4H), 2.50-2.45 (m, 4H), 2.27-2.18 (m, 1H), 1.98-1.88 (m, 2H), 1.80-1.67 (m, 2H), 1.60-1.47 (m, 4H).

### Example 61: Preparation of 4-((1s,3s)-3-(dimethylamino)cyclobutoxy)-2-methoxyaniline (Intermediate

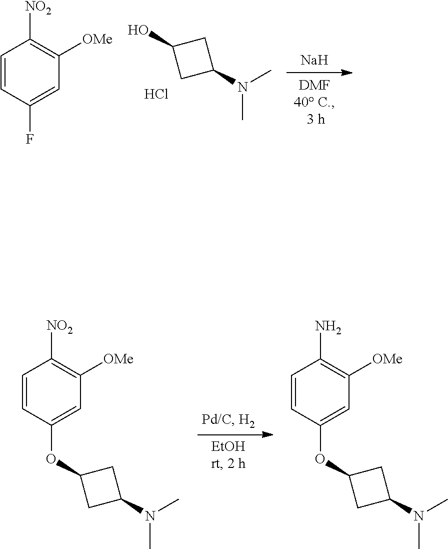

The title compound was prepared following general procedure G, starting from 4-fluoro-2-methoxy-1-nitrobenzene and (1s,3s)-3-(dimethylamino)cyclobutan-1-ol in DMF followed by nitro reduction employing the general nitro reduction procedure H. MS (m/z): [M+H]⁺: 237.0.

### Example 62: Preparation of Compound 19

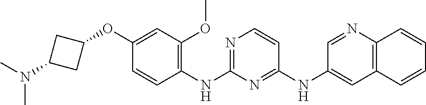 [SMILES: COc1cc(O[C@H]2C[C@@H](N(C)C)C2)ccc1Nc1nccc(Nc2cnc3ccccc3c2)n1]

Compound 19 was prepared from the corresponding intermediate according to general procedure E. MS (m/z): [M+H]⁺ 457.5. ¹H NMR (400 MHz, DMSO) δ (ppm): 9.75 (s, 1H), 8.81 (d, J=2.5 Hz, 1H), 8.76 (s, 1H), 8.03-7.99 (m, 2H), 7.89 (d, J=7.9 Hz, 1H), 7.64-7.45 (m, 4H), 6.61 (d, J=2.6 Hz, 1H), 6.44 (dd, J=8.6, 2.6 Hz, 1H), 6.23 (d, J=5.7 Hz, 1H), 4.46 (p, J=7.1 Hz, 1H), 3.74 (s, 3H), 2.72-2.60 (m, 2H), 2.42-2.33 (m, 1H), 2.07 (s, 6H), 1.94-1.80 (m, 2H).

### Example 63: Preparation of 2-methoxy-4-(3-(piperidin-1-yl)propoxy)aniline (Intermediate 16)

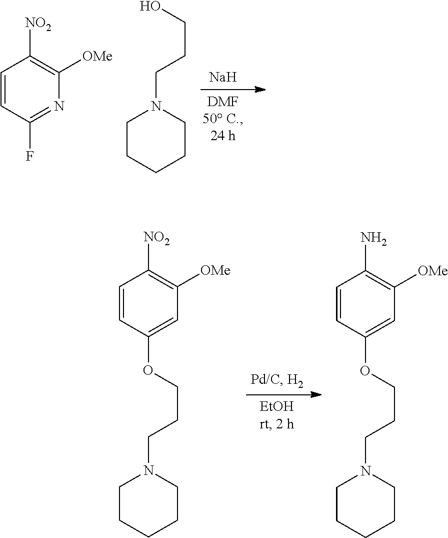

The title compound was prepared following general procedure G, starting from 4-fluoro-2-methoxy-1-nitrobenzene and 3-(piperidin-1-yl)propan-1-ol in DMF followed by nitro reduction employing the general nitro reduction procedure H. MS (m/z): [M+H]⁺: 265.4.

### Example 64: Preparation of Compound 20

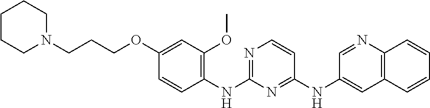 [SMILES: COc1cc(OCCCN2CCCCC2)ccc1Nc1nccc(Nc2cnc3ccccc3c2)n1]

Compound 20 was prepared from the corresponding intermediate according to general procedure E. MS (m/z): [M+H]⁺ 485.6. ¹H NMR (400 MHz, DMSO) δ (ppm): 9.75 (s, 1H), 8.80 (d, J=2.5 Hz, 1H), 8.76 (s, 1H), 8.05-7.99 (m, 2H), 7.89 (d, J=8.4 Hz, 1H), 7.64-7.46 (m, 4H), 6.67 (d, J=2.6 Hz, 1H), 6.54 (dd, J=8.7, 2.7 Hz, 1H), 6.23 (d, J=5.7 Hz, 1H), 4.05 (t, J=6.4 Hz, 2H), 3.75 (s, 3H), 2.43 (t, J=7.2 Hz, 2H), 2.39-2.26 (m, 4H), 1.97-1.87 (m, 2H), 1.56-1.46 (m, 4H), 1.43-1.34 (m, 2H).

### Example 65: Preparation of 2-methoxy-4-(3-(piperidin-1-yl)propoxy)aniline (Intermediate 17)

### Step 1: Preparation of (1s,3s)-3-morpholinocyclobutanol

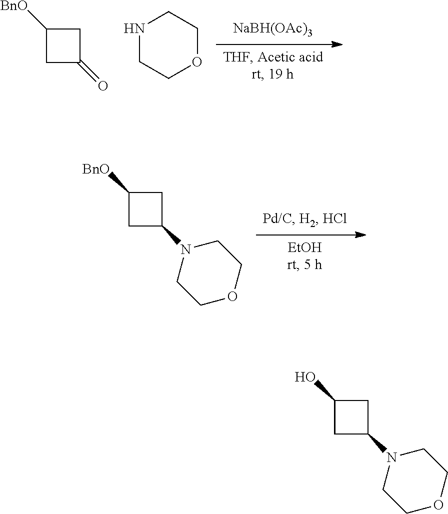

The title compound was prepared following general procedure U with morpholine. MS (m/z): [M+H]⁺: 158.1.

### Step 2: Preparation of 3-methoxy-4-((1s,3s)-3-morpholinocyclobutoxy)aniline

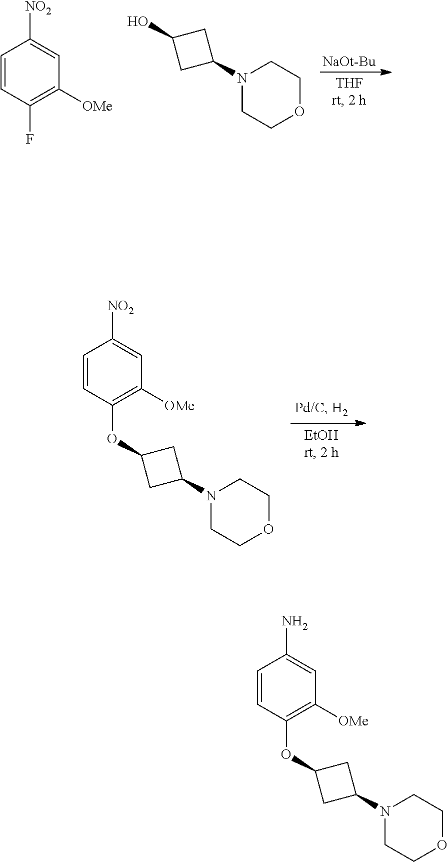

The title compound was prepared following general procedure G, starting from 4-fluoro-2-methoxy-1-nitrobenzene and (1s,3s)-3-morpholinocyclobutan-1-ol in DMF followed by nitro reduction employing the general nitro reduction procedure H. MS (m/z): [M+H]⁺: 309.0 (Nitro intermediate).

### Example 66: Preparation of Compound 21

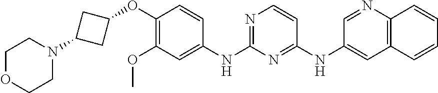 [SMILES: COc1cc(Nc2nccc(Nc3cnc4ccccc4c3)n2)ccc1O[C@H]1C[C@@H](N2CCOCC2)C1]

Compound 21 was prepared from the corresponding intermediate according to general procedure E. MS (m/z): [M+H]⁺ 499.5. H NMR (500 MHz, DMSO) δ (ppm): 9.82 (s, 1H), 9.07 (s, 1H), 8.95 (s, 1H), 8.92 (d, J=2.5 Hz, 1H), 8.08 (d, J=5.7 Hz, 1H), 7.92 (d, J=8.2 Hz, 1H), 7.74 (d, J=6.9 Hz, 1H), 7.59 (ddd, J=8.4, 6.9, 1.7 Hz, 1H), 7.56-7.51 (m, 1H), 7.27 (s, 1H), 7.22 (d, J=8.7 Hz, 1H), 6.76 (d, J=8.7 Hz, 1H), 6.30 (d, J=5.7 Hz, 1H), 4.40 (p, J=7.1 Hz, 1H), 3.63 (s, 3H), 3.61-3.54 (m, 4H), 2.58 (ddd, J=9.2, 8.0, 4.7 Hz, 2H), 2.47-2.38 (m, 1H), 2.28 (brm, 4H), 1.93-1.81 (m, 2H).

### Example 67: Preparation of 3-methoxy-4-((1r,3r)-3-morpholinocyclobutoxy)aniline (Intermediate 18)

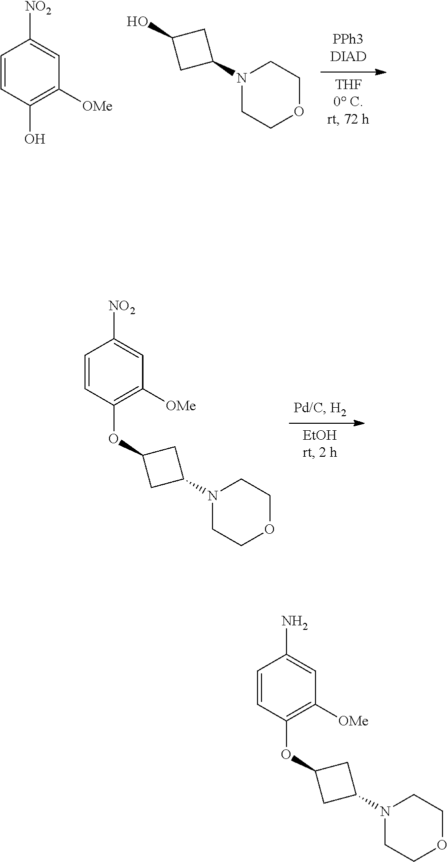

The title compound was prepared following general procedure T, starting from 2-methoxy-4-nitrophenol and (1s,3s)-3-morpholinocyclobutan-1-ol in THF followed by nitro reduction employing the general nitro reduction procedure H. MS (m/z): [M+H]⁺: 278.8.

### Example 68: Preparation of Compound 22

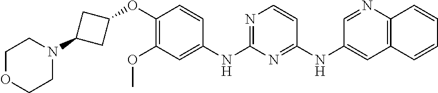 [SMILES: COc1cc(Nc2nccc(Nc3cnc4ccccc4c3)n2)ccc1O[C@H]1C[C@H](N2CCOCC2)C1]

Compound 22 was prepared from the corresponding intermediate according to general procedure E. MS (m/z): [M+H]⁺ 499.5. H NMR (400 MHz, DMSO) δ (ppm): 9.81 (s, 1H), 9.07 (s, 1H), 8.95 (s, 1H), 8.92 (d, J=2.5 Hz, 1H), 8.08 (d, J=5.7 Hz, 1H), 7.92 (d, J=8.1 Hz, 1H), 7.74 (d, J=8.2 Hz, 1H), 7.65-7.49 (m, 2H), 7.28 (s, 1H), 7.22 (d, J=8.8 Hz, 1H), 6.68 (d, J=8.7 Hz, 1H), 6.29 (d, J=5.7 Hz, 1H), 4.73-4.64 (m, 1H), 3.64 (s, 3H), 3.63-3.58 (m, 4H), 2.96-2.85 (m, 1H), 2.39-2.23 (m, 6H), 2.20-2.09 (m, 2H).

### Example 69: Preparation of 4-chloro-N-(3-methoxy-4-((1r,3r)-3-morpholinocyclobutoxy)phenyl)pyrimidin-2-amine (Intermediate 19)

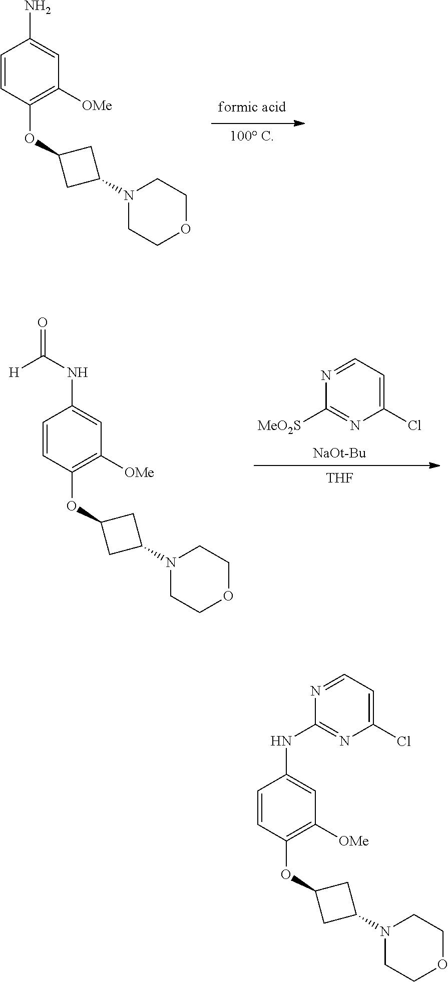

The title compound was prepared following general procedure A, starting from 3-methoxy-4-((1r,3r)-3-morpholinocyclobutoxy)aniline and 4-chloro-2-(methylsulfonyl)pyrimidine. MS (m/z): [M+H]⁺: 391.6.

### Example 70: Preparation of Compound 23

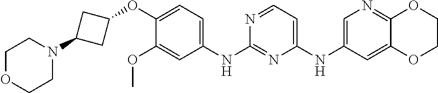 [SMILES: COc1cc(Nc2nccc(Nc3cnc4c(c3)OCCO4)n2)ccc1O[C@H]1C[C@H](N2CCOCC2)C1]

Compound 23 was prepared from the corresponding intermediate according to general procedure C. MS (m/z): [M+H]⁺ 507.4. ¹H NMR (500 MHz, DMSO): δ (ppm): 9.28 (s, 1H), 8.96 (s, 1H), 8.01-7.94 (m, 2H), 7.78 (d, J=2.3 Hz, 1H), 7.29 (s, 1H), 7.20 (dd, J=8.7, 2.1 Hz, 1H), 6.63 (d, J=8.8 Hz, 1H), 6.11 (d, J=5.7 Hz, 1H), 4.67-4.59 (m, 1H), 4.39-4.33 (m, 2H), 4.26-4.22 (m, 2H), 3.67 (s, 3H), 3.58 (t, J=4.6 Hz, 4H), 2.92-2.84 (m, 1H), 2.35-2.22 (m, 6H), 2.11 (ddd, J=15.5, 7.2, 3.4 Hz, 2H).

### Example 71: Preparation of 4-((1r,3r)-3-(4-amino-2-methoxyphenoxy)cyclobutyl)thiomorpholine 1,1-dioxide (Intermediate 20)

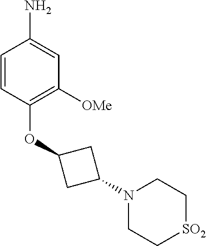 [SMILES: COc1cc(N)ccc1O[C@H]1C[C@H](N2CCS(=O)(=O)CC2)C1]

### Step 1: 4-((1s,3s)-3-hydroxycyclobutyl)thiomorpholine 1,1-dioxide

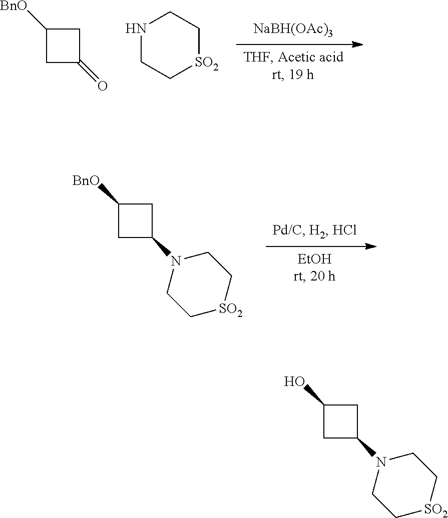

The title compound was prepared following general procedure U, starting from 3-(benzyloxy)cyclobutanone and thiomorpholine 1,1-dioxide. MS (m/z): [M+H]⁺: 296.2 (benzyl intermediate).

### Step 2: 4-((1r,3r)-3-(4-amino-2-methoxyphenoxy)cyclobutyl)thiomorpholine 1,1-dioxide

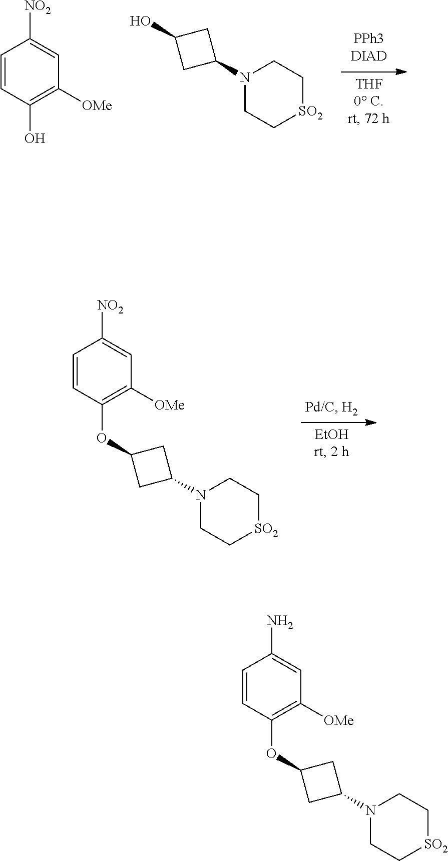

The title compound was prepared following general procedure T, starting from 2-methoxy-4-nitrophenol and 4-((1s,3s)-3-hydroxycyclobutyl)thiomorpholine 1,1-dioxide in THF followed by nitro reduction employing the general nitro reduction procedure H. MS (m/z): [M+H]⁺: 327.2.

### Example 72: Preparation of Compound 24

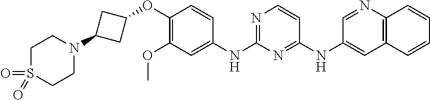 [SMILES: COc1cc(Nc2nccc(Nc3cnc4ccccc4c3)n2)ccc1O[C@H]1C[C@H](N2CCS(=O)(=O)CC2)C1]

Compound 24 was prepared from the corresponding intermediate according to general procedure E. MS (m/z): [M+H]⁺ 547.4. ¹H NMR (400 MHz, DMSO): δ (ppm): 9.81 (s, 1H), 9.07 (s, 1H), 8.95 (s, 1H), 8.93 (d, J=2.5 Hz, 1H), 8.08 (d, J=5.7 Hz, 1H), 7.93 (d, J=8.2 Hz, 1H), 7.74 (d, J=7.1 Hz, 1H), 7.60 (ddd, J=8.4, 6.9, 1.6 Hz, 1H), 7.57-7.51 (m, 1H), 7.29 (s, 1H), 7.23 (dd, J=8.9, 1.8 Hz, 1H), 6.67 (d, J=8.7 Hz, 1H), 6.30 (d, J=5.7 Hz, 1H), 4.71-4.63 (m, 1H), 3.64 (s, 3H), 3.24 (dd, J=13.8, 6.5 Hz, 1H), 3.14-3.07 (m, 4H), 2.79 (d, J=3.8 Hz, 4H), 2.37-2.27 (m, 2H), 2.27-2.15 (m, 2H).

### Example 73: Preparation of 4-((1r,3r)-3-(4-((4-chloropyrimidin-2-yl)amino)-2-methoxyphenoxy)cyclobutyl)thiomorpholine 1,1-dioxide (Intermediate 21)

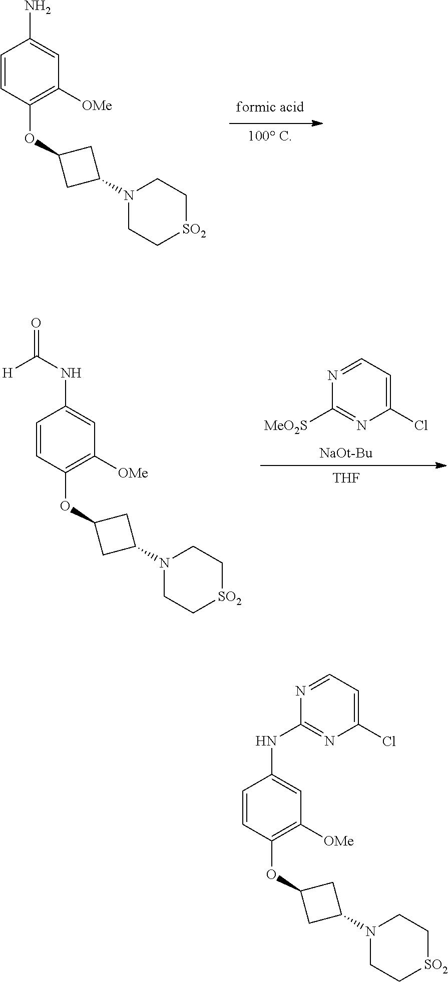

The title compound was prepared following general procedure A, starting from 4-((1r,3r)-3-(4-amino-2-methoxyphenoxy)cyclobutyl)thiomorpholine 1,1-dioxide and 4-chloro-2-(methylsulfonyl)pyrimidine. MS (m/z): [M+H]⁺: 355.1 (Formylated intermediate).

### Example 74: Preparation of Compound 25

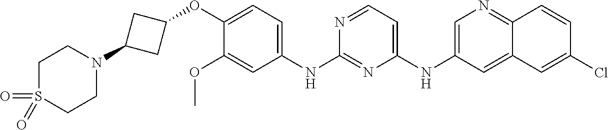 [SMILES: COc1cc(Nc2nccc(Nc3cnc4ccc(Cl)cc4c3)n2)ccc1O[C@H]1C[C@H](N2CCS(=O)(=O)CC2)C1]

Compound 25 was prepared from the corresponding intermediate according to general procedure C. MS (m/z): [M+H]⁺ 581.1, 583.5. H NMR (500 MHz, DMSO): δ (ppm): 9.93 (s, 1H), 9.07 (s, 1H), 9.00 (s, 1H), 8.92 (d, J=2.4 Hz, 1H), 8.10 (d, J=5.7 Hz, 1H), 7.93 (d, J=8.9 Hz, 1H), 7.81 (s, 1H), 7.58 (dd, J=8.9, 2.4 Hz, 1H), 7.28 (s, 1H), 7.22 (d, J=8.9 Hz, 1H), 6.69 (d, J=8.7 Hz, 1H), 6.31 (d, J=5.7 Hz, 1H), 4.73-4.65 (m, 1H), 3.67 (s, 3H), 3.26-3.21 (m, 1H), 3.11-3.08 (m, 4H), 2.81-2.75 (m, 4H), 2.35-2.26 (m, 2H), 2.26-2.17 (m, 2H).

### Example 75: Preparation of 4-chloro-N-(4-((1r,3r)-3-morpholinocyclobutoxy)phenyl)pyrimidin-2-amine (Intermediate 22)

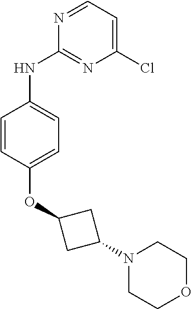 [SMILES: Clc1ccnc(Nc2ccc(O[C@H]3C[C@H](N4CCOCC4)C3)cc2)n1]

### Step 1: 3-methoxy-4-((1r,3r)-3-morpholinocyclobutoxy)aniline

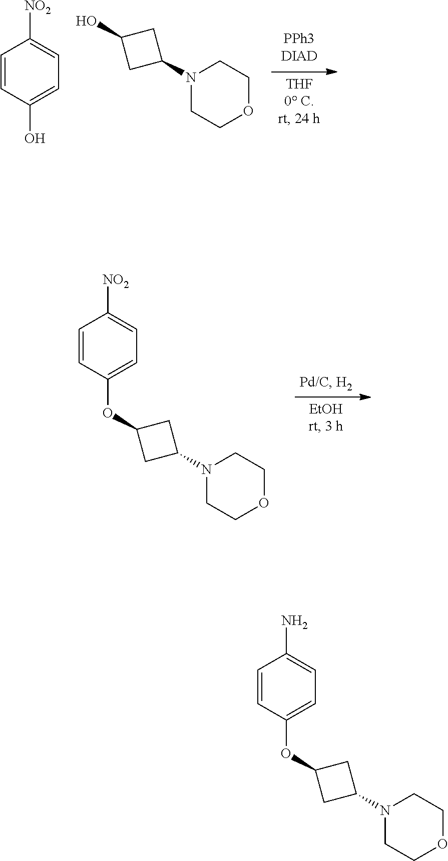

The title compound was prepared following general procedure T, starting from 4-nitrophenol and (1s,3s)-3-morpholinocyclobutan-1-ol in THF followed by nitro reduction employing the general nitro reduction procedure H. MS (m/z): [M+H]⁺: 249.3.

### Step 2: 4-chloro-N-(4-((1r,3r)-3-morpholinocyclobutoxy)phenyl)pyrimidin-2-amine

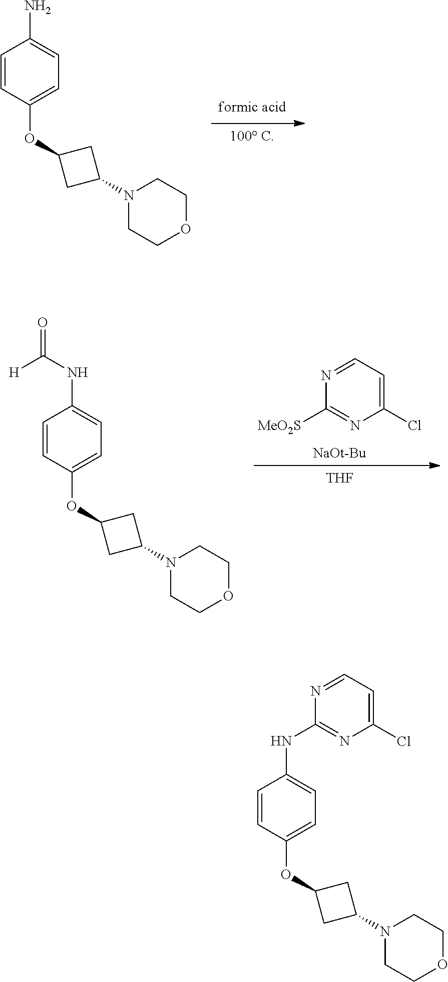

The title compound was prepared following general procedure A, starting from 4-((1r,3r)-3-morpholinocyclobutoxy)aniline and 4-chloro-2-(methylsulfonyl)pyrimidine. MS (m/z): [M+H]⁺: 361.4.

### Example 76: Preparation of Compound 26

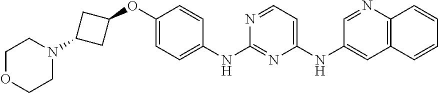 [SMILES: c1ccc2ncc(Nc3ccnc(Nc4ccc(O[C@H]5C[C@H](N6CCOCC6)C5)cc4)n3)cc2c1]

Compound 26 was prepared from the corresponding intermediate according to general procedure C. MS (m/z): [M+H]⁺ 469.4. H NMR (400 MHz, DMSO): δ (ppm): 9.80 (s, 1H), 9.07 (s, 1H), 8.95-8.87 (m, 2H), 8.06 (d, J=5.7 Hz, 1H), 7.93 (d, J=8.2 Hz, 1H), 7.75 (d, J=5.8 Hz, 1H), 7.63-7.50 (m, 4H), 6.82-6.72 (m, 2H), 6.28 (d, J=5.7 Hz, 1H), 4.75-4.67 (m, 1H), 3.61 (t, J=4.6 Hz, 4H), 2.97-2.86 (m, 1H), 2.43-2.34 (m, 2H), 2.31 (s, 4H), 2.19-2.10 (m, 2H).

### Example 77: Preparation of 4-chloro-N-(4-((1r,3r)-3-(dimethylamino)cyclobutoxy)phenyl)pyrimidin-2-amine (Intermediate 23)

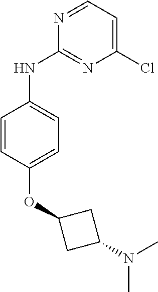 [SMILES: CN(C)[C@H]1C[C@H](Oc2ccc(Nc3nccc(Cl)n3)cc2)C1]

### Step 1: 4-((1r,3r)-3-(dimethylamino)cyclobutoxy)aniline

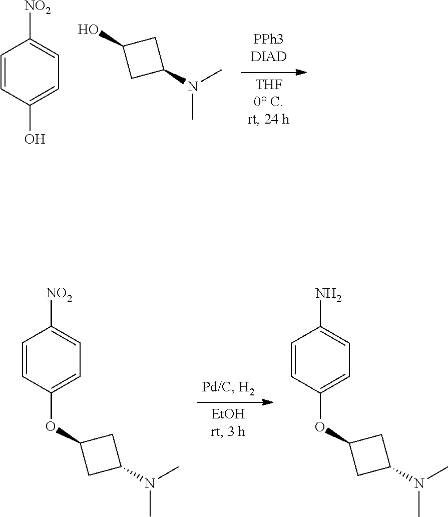

The title compound was prepared following general procedure T, starting from 4-nitrophenol and (1s,3s)-3-(dimethylamino)cyclobutan-1-ol in THF followed by nitro reduction employing the general nitro reduction procedure H. MS (m/z): [M+H]⁺: 207.3.

### Step 2: 4-chloro-N-(4-((1r,3r)-3-(dimethylamino)cyclobutoxy)phenyl)pyrimidin-2-amine

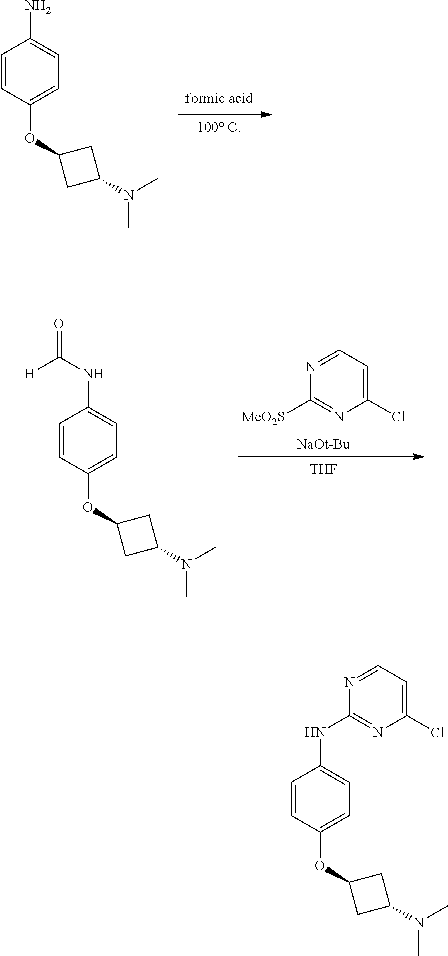

The title compound was prepared following general procedure A, starting from 4-((1r,3r)-3-(dimethylamino)cyclobutoxy)aniline and 4-chloro-2-(methylsulfonyl)pyrimidine. MS (m/z): [M+H]⁺: 319.3.

### Example 78: Preparation of Compound 27

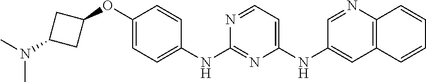 [SMILES: CN(C)[C@H]1C[C@H](Oc2ccc(Nc3nccc(Nc4cnc5ccccc5c4)n3)cc2)C1]

Compound 27 was prepared from the corresponding intermediate according to general procedure C. MS (m/z): [M+H]⁺ 427.6. ¹H NMR (500 MHz, DMSO): δ (ppm): 9.80 (s, 1H), 9.07 (s, 1H), 8.92 (s, 1H), 8.90 (s, 1H), 8.06 (d, J=5.7 Hz, 1H), 7.93 (d, J=8.3 Hz, 1H), 7.75 (d, J=5.7 Hz, 1H), 7.63-7.51 (m, 4H), 6.79-6.72 (m, 2H), 6.28 (d, J=5.7 Hz, 1H), 4.72-4.66 (m, 1H), 2.87-2.79 (m, 1H), 2.38-2.29 (m, 2H), 2.18-2.11 (m, 2H), 2.08 (s, 6H).

### Example 79: Preparation of 4-chloro-N-(4-((1s,3s)-3-morpholinocyclobutoxy)phenyl)pyrimidin-2-amine (Intermediate 24)

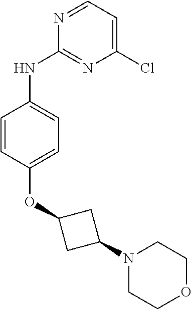 [SMILES: Clc1ccnc(Nc2ccc(O[C@H]3C[C@@H](N4CCOCC4)C3)cc2)n1]

### Step 1: 4-((1s,3s)-3-morpholinocyclobutoxy)aniline

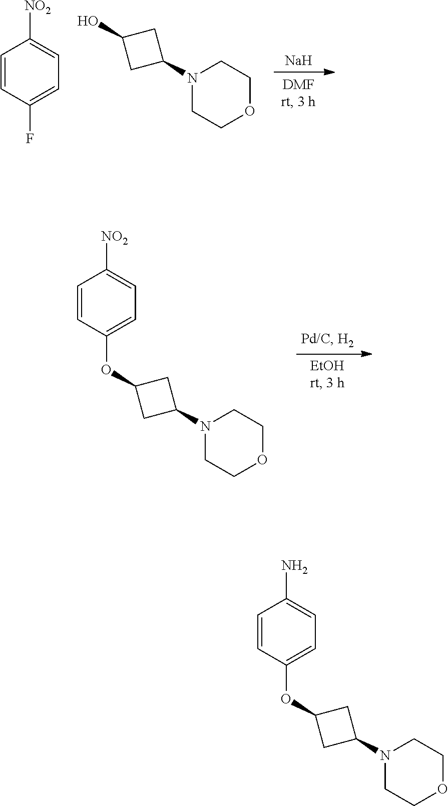

The title compound was prepared following general procedure G, starting from 1-fluoro-4-nitrobenzene and (1s,3s)-3-morpholinocyclobutan-1-ol in DMF followed by nitro reduction employing the general nitro reduction procedure H. MS (m/z): [M+H]⁺: 279.2 (Nitro intermediate).

### Step 2: 4-chloro-N-(4-((1s,3s)-3-morpholinocyclobutoxy)phenyl)pyrimidin-2-amine

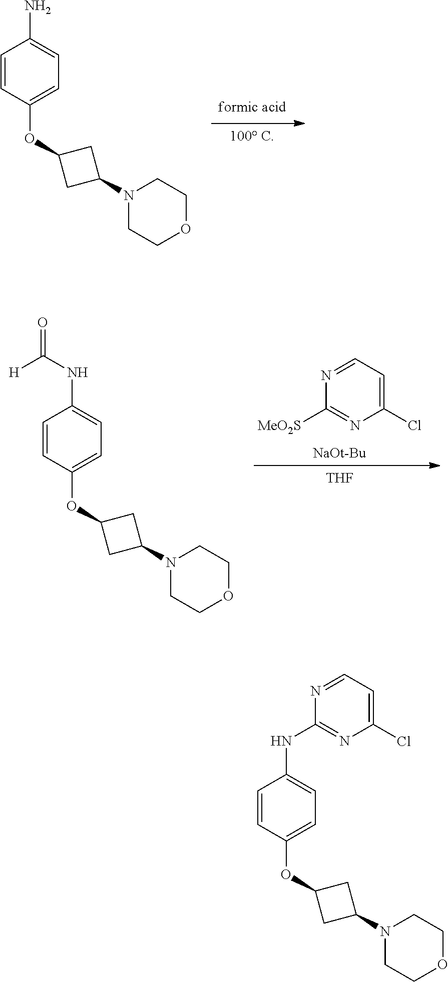

The title compound was prepared following general procedure A, starting from 4-((1s,3s)-3-morpholinocyclobutoxy)aniline and 4-chloro-2-(methylsulfonyl)pyrimidine. MS (m/z): [M+H]⁺: 361.2.

### Example 80: Preparation of Compound 28

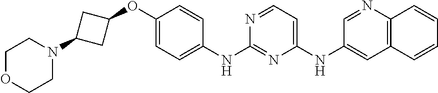 [SMILES: c1ccc2ncc(Nc3ccnc(Nc4ccc(O[C@H]5C[C@@H](N6CCOCC6)C5)cc4)n3)cc2c1]

Compound 28 was prepared from the corresponding intermediate according to general procedure C. MS (m/z): [M+H]⁺ 469.5. ¹H NMR (500 MHz, DMSO): δ (ppm): 9.79 (s, 1H), 9.07 (s, 1H), 8.92 (s, 1H), 8.90 (d, J=2.2 Hz, 1H), 8.06 (d, J=5.7 Hz, 1H), 7.93 (d, J=8.2 Hz, 1H), 7.75 (d, J=5.7 Hz, 1H), 7.63-7.49 (m, 4H), 6.80-6.72 (m, 2H), 6.28 (d, J=5.7 Hz, 1H), 4.75-4.67 (m, 1H), 3.61 (t, J=4.5 Hz, 4H), 2.96-2.88 (m, 1H), 2.42-2.35 (m, 2H), 2.31 (s, 4H), 2.19-2.10 (m, 2H).

### Example 81: Preparation of 4-chloro-N-(4-(((1R,5S,7s)-9-methyl-3-oxa-9-azabicyclo[3.3.1]nonan-7-yl)oxy)phenyl)pyrimidin-2-amine (Intermediate 25)

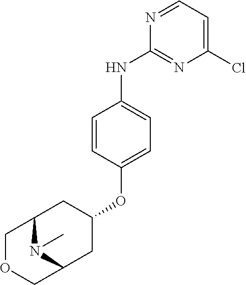 [SMILES: CN1[C@@H]2COC[C@H]1C[C@@H](Oc1ccc(Nc3nccc(Cl)n3)cc1)C2]

### Step 1: 4-(((1R,5S,7s)-9-methyl-3-oxa-9-azabicyclo[3.3.1]nonan-7-yl)oxy)aniline

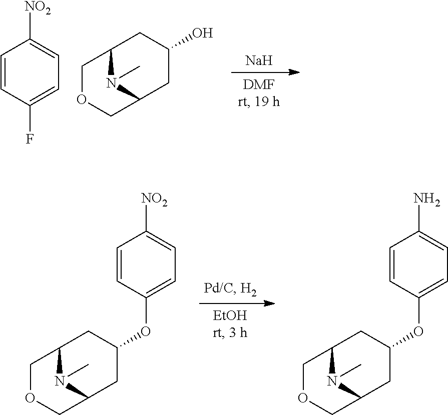

The title compound was prepared following general procedure G, starting from 1-fluoro-4-nitrobenzene and (1R,5S,7s)-9-methyl-3-oxa-9-azabicyclo[3.3.1]nonan-7-ol in DMF followed by nitro reduction employing the general nitro reduction procedure H. MS (m/z): [M+H]⁺: 279.2 (Nitro intermediate).

### Step 2: 4-chloro-N-(4-(((1R,5S,7s)-9-methyl-3-oxa-9-azabicyclo[3.3.1]nonan-7-yl)oxy)phenyl)pyrimidin-2-amine

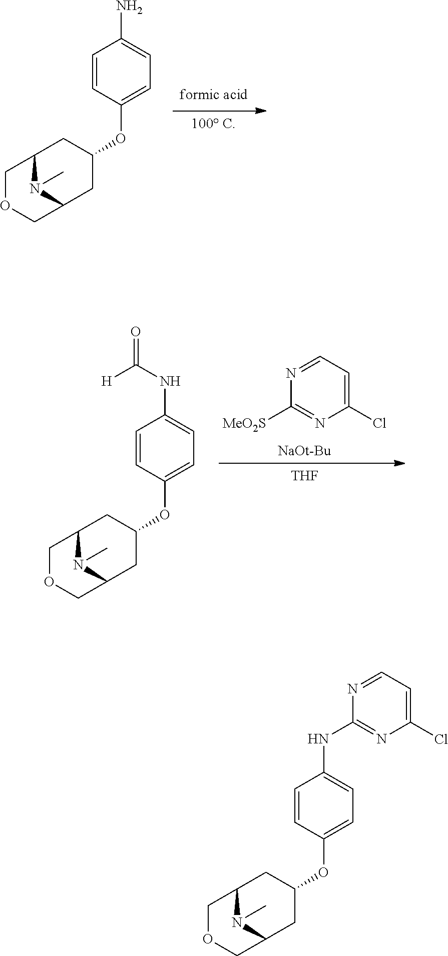

The title compound was prepared following general procedure A, starting from 4-(((1R,5S,7s)-9-methyl-3-oxa-9-azabicyclo[3.3.1]nonan-7-yl)oxy)aniline and 4-chloro-2-(methylsulfonyl)pyrimidine. MS (m/z): [M+H]⁺: 361.4.

### Example 82: Preparation of Compound 29

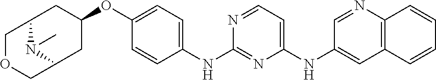 [SMILES: CN1[C@@H]2COC[C@H]1C[C@@H](Oc1ccc(Nc3nccc(Nc4cnc5ccccc5c4)n3)cc1)C2]

Compound 29 was prepared from the corresponding intermediate according to general procedure C. MS (m/z): [M+H]⁺ 469.4. ¹H NMR (500 MHz, DMSO): δ (ppm): 9.80 (s, 1H), 9.07 (s, 1H), 8.95 (s, 1H), 8.90 (d, J=2.4 Hz, 1H), 8.07 (d, J=5.7 Hz, 1H), 7.92 (d, J=8.3 Hz, 1H), 7.76 (d, J=6.9 Hz, 1H), 7.59 (ddd, J=8.4, 6.9, 1.6 Hz, 1H), 7.56-7.49 (m, 3H), 6.89-6.82 (m, 2H), 6.28 (d, J=5.7 Hz, 1H), 4.65 (p, J=6.1 Hz, 1H), 3.80 (dd, J=10.9, 2.2 Hz, 2H), 3.38 (d, J=11.0 Hz, 2H), 2.69 (d, J=8.2 Hz, 2H), 2.44 (s, 3H), 2.43-2.37 (m, 2H), 1.63 (dd, J=14.5, 3.2 Hz, 2H).

### Example 83: Preparation of 4-chloro-N-(4-(((1R,5S,7r)-9-methyl-3-oxa-9-azabicyclo[3.3.1]nonan-7-yl)oxy)phenyl)pyrimidin-2-amine (Intermediate 26)

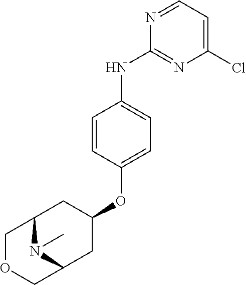 [SMILES: CN1[C@@H]2COC[C@H]1C[C@H](Oc1ccc(Nc3nccc(Cl)n3)cc1)C2]

### Step 1: 4-(((1R,5S,7r)-9-methyl-3-oxa-9-azabicyclo[3.3.1]nonan-7-yl)oxy)aniline

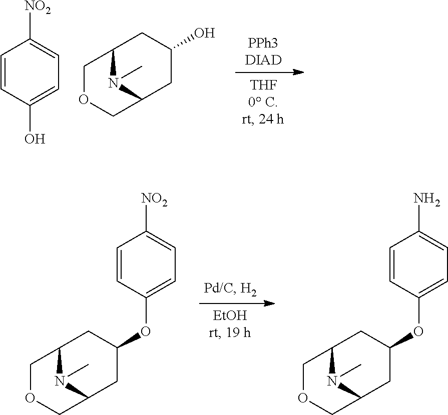

The title compound was prepared following general procedure T, starting from 4-nitrophenol and (1R,5S,7s)-9-methyl-3-oxa-9-azabicyclo[3.3.1]nonan-7-ol in THF followed by nitro reduction employing the general nitro reduction procedure H. MS (m/z): [M+H]⁺: 279.2 (Nitro intermediate).

### Step 2: 4-chloro-N-(4-(((1R,5S,7r)-9-methyl-3-oxa-9-azabicyclo[3.3.1]nonan-7-yl)oxy)phenyl)pyrimidin-2-amine

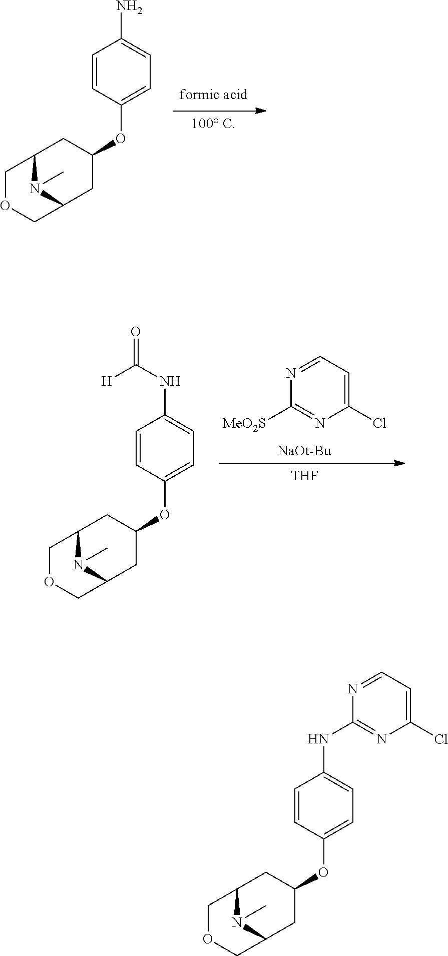

The title compound was prepared following general procedure A, starting from 4-(((1R,5S,7r)-9-methyl-3-oxa-9-azabicyclo[3.3.1]nonan-7-yl)oxy)aniline and 4-chloro-2-(methylsulfonyl)pyrimidine. MS (m/z): [M+H]⁺: 361.2.

### Example 84: Preparation of Compound 30

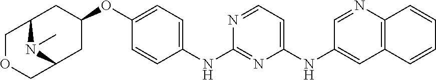 [SMILES: CN1[C@@H]2COC[C@H]1C[C@H](Oc1ccc(Nc3nccc(Nc4cnc5ccccc5c4)n3)cc1)C2]

Compound 30 was prepared from the corresponding intermediate according to general procedure C. MS (m/z): [M+H]⁺ 469.5. H NMR (500 MHz, DMSO): δ 9.80 (s, 1H), 9.08 (s, 1H), 8.93 (s, 1H), 8.90 (d, J=2.3 Hz, 1H), 8.07 (d, J=5.7 Hz, 1H), 7.92 (d, J=8.2 Hz, 1H), 7.76 (d, J=8.1 Hz, 1H), 7.61-7.51 (m, 4H), 6.91-6.81 (m, 2H), 6.28 (d, J=5.7 Hz, 1H), 5.23-5.12 (m, 1H), 3.75-3.67 (m, 4H), 2.76 (s, 2H), 2.44 (s, 3H), 2.00-1.92 (m, 4H).

### Example 85: Preparation of 4-chloro-N-(6-(3-morpholinopropoxy)pyridin-3-yl)pyrimidin-2-amine (Intermediate 27)

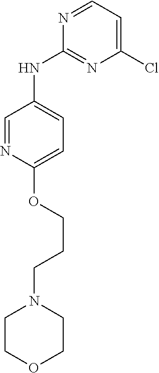 [SMILES: Clc1ccnc(Nc2ccc(OCCCN3CCOCC3)nc2)n1]

### Step 1: 6-(3-morpholinopropoxy)pyridin-3-amine

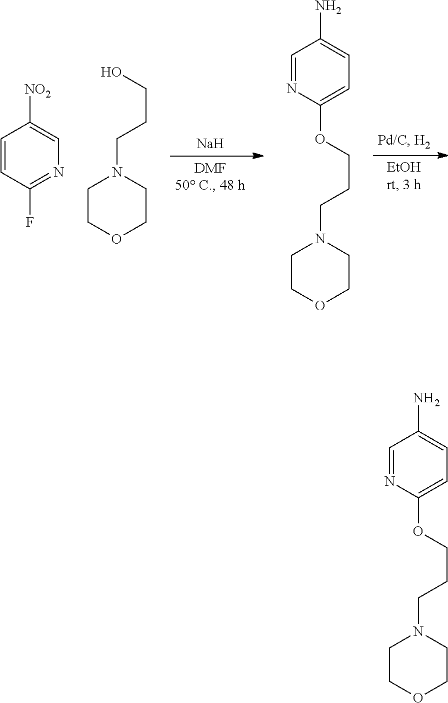

The title compound was prepared following general procedure G, starting from 2-fluoro-5-nitropyridine and 3-(piperidin-1-yl)propan-1-ol in DMF followed by nitro reduction employing the general nitro reduction procedure H. MS (m/z): [M+H]⁺: 237.9.

### Step 2: 4-chloro-N-(6-(3-morpholinopropoxy)pyridin-3-yl)pyrimidin-2-amine

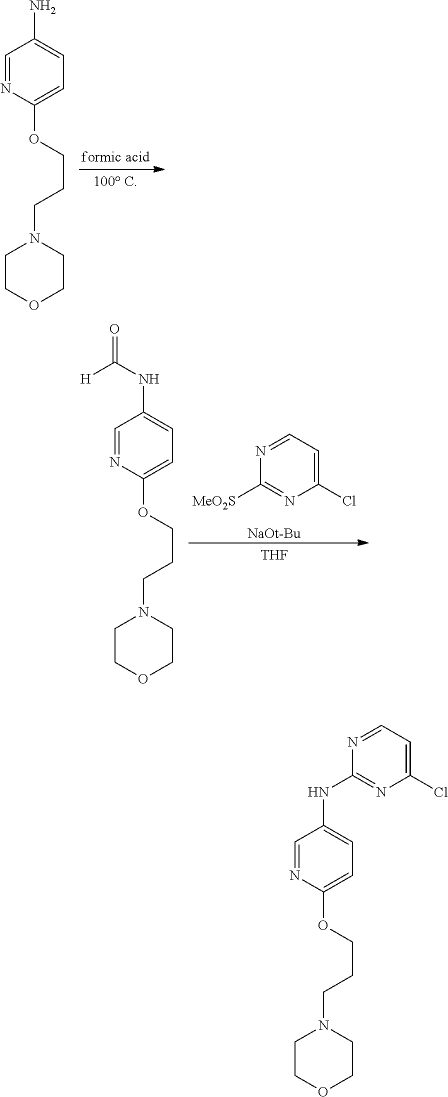 [SMILES: Nc1ccc(OCCCN2CCOCC2)nc1 ; O=CNc1ccc(OCCCN2CCOCC2)nc1 ; CS(=O)(=O)c1nccc(Cl)n1 ; "THF" ; Clc1ccnc(Nc2ccc(OCCCN3CCOCC3)nc2)n1 ; "NaOt-Bu"]

The title compound was prepared following general procedure A, starting from 6-(3-morpholinopropoxy)pyridin-3-amine and 4-chloro-2-(methylsulfonyl)pyrimidine. MS (m/z): [M+H]⁺: 349.9.

### Example 86: Preparation of Compound 31

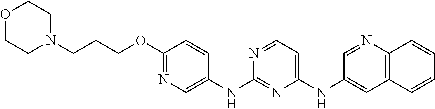 [SMILES: c1ccc2ncc(Nc3ccnc(Nc4ccc(OCCCN5CCOCC5)nc4)n3)cc2c1]

Compound 31 was prepared from the corresponding intermediate according to general procedure C. MS (m/z): [M+H]⁺ 458.3. H NMR (500 MHz, DMSO): δ 9.86 (s, 1H), 9.17 (s, 1H), 8.90 (s, 2H), 8.44 (d, J=2.8 Hz, 1H), 8.08 (d, J=5.7 Hz, 1H), 7.98-7.87 (m, 2H), 7.80 (d, J=7.7 Hz, 1H), 7.68-7.47 (m, 2H), 6.77 (d, J=9.2 Hz, 1H), 6.32 (d, J=5.7 Hz, 1H), 4.29 (t, J=6.6 Hz, 2H), 3.60-3.54 (m, 4H), 2.43 (t, J=7.2 Hz, 2H), 2.41-2.30 (m, 4H), 1.90 (p, J=6.8 Hz, 2H).

### Example 87: Preparation of 4-chloro-N-(3-methoxy-4-(((1s,4s)-4-morpholinocyclohexyl)oxy)phenyl)pyrimidin-2-amine (Intermediate 28)

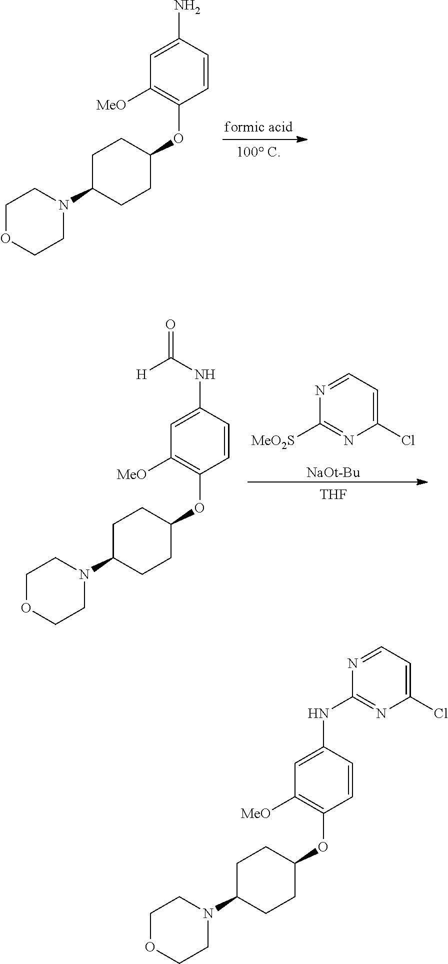 [SMILES: COc1cc(N)ccc1O[C@H]1CC[C@@H](N2CCOCC2)CC1 ; COc1cc(NC=O)ccc1O[C@H]1CC[C@@H](N2CCOCC2)CC1 ; CS(=O)(=O)c1nccc(Cl)n1 ; "THF" ; COc1cc(Nc2nccc(Cl)n2)ccc1O[C@H]1CC[C@@H](N2CCOCC2)CC1 ; "NaOt-Bu"]

The title compound was prepared following general procedure A, starting from 3-methoxy-4-(((1s,4s)-4-morpholinocyclohexyl)oxy)aniline and 4-chloro-2-(methylsulfonyl)pyrimidine. MS (m/z): [M+H]⁺: 419.1.

### Example 88: Preparation of Compound 32

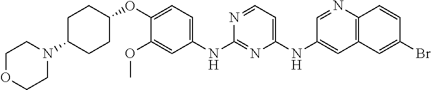 [SMILES: COc1cc(Nc2nccc(Nc3cnc4ccc(Br)cc4c3)n2)ccc1O[C@H]1CC[C@@H](N2CCOCC2)CC1]

Compound 32 was prepared from the corresponding intermediate according to general procedure C. MS (m/z): [M+H]⁺ 605.3, 607.1. H NMR (400 MHz, DMSO): δ 9.94 (s, 1H), 9.10 (s, 1H), 9.03 (s, 1H), 8.94 (d, J=2.5 Hz, 1H), 8.11 (d, J=5.7 Hz, 1H), 8.04 (s, 1H), 7.86 (d, J=8.9 Hz, 1H), 7.69 (dd, J=8.9, 2.2 Hz, 1H), 7.32 (s, 1H), 7.26 (dd, J=8.4, 2.0 Hz, 1H), 6.90 (d, J=8.7 Hz, 1H), 6.32 (d, J=5.7 Hz, 1H), 4.39-4.31 (m, 1H), 3.68 (s, 3H), 3.62-3.54 (m, 4H), 2.48-2.43 (m, 4H), 2.25-2.16 (m, 1H), 1.97-1.88 (m, 2H), 1.78-1.65 (m, 2H), 1.60-1.47 (m, 4H).

### Example 89: Preparation of 4-chloro-N-(4-(((1s,4s)-4-(dimethylamino)cyclohexyl)oxy)-3-methoxyphenyl)pyrimidin-2-amine (Intermediate 29)

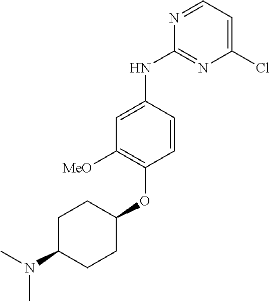 [SMILES: COc1cc(Nc2nccc(Cl)n2)ccc1O[C@H]1CC[C@@H](N(C)C)CC1]

### Step 1: (1s,4s)-4-(dimethylamino)cyclohexanol

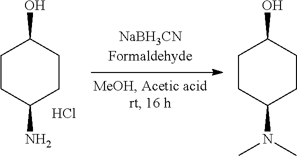

Into a solution of cis-4-aminocyclohexanol hydrochloride (2.50 g, 16.2 mmol) in MeOH (125 mL) was added formaldehyde (37 wt % in water, 2.65 mL, 32.7 mmol), sodium cyanoborohydride (2.14 g, 32.3 mmol) and acetic acid (4.50 mL, 78.7 mmol). After stirring for 16 h, the reaction was quenched 20 mL of 6M NaOH and concentrated under reduced pressure to remove MeOH. The resulting brown solution was diluted with 100 mL of water and extracted with DCM three times. The aqueous phase was further extracted with chloroform/IPA (3:1) three times. The combined organics were dried and concentrated to give the title compound (2.46 g, 100%) as a brown oil.

### Step 2: 4-(((1s,4s)-4-dimethylamino)cyclohexyl)oxy)-3-methoxyaniline

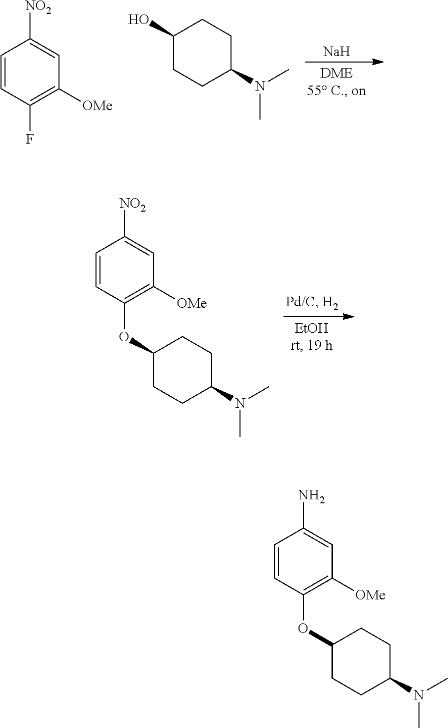

The title compound was prepared following general procedure G, starting from 1-fluoro-2-methoxy-4-nitrobenzene and (1s,4s)-4-(dimethylamino)cyclohexanol in DME followed by nitro reduction employing the general nitro reduction procedure H. MS (m/z): [M+H]⁺: 265.0.

### Step 3: 4-chloro-N-(4-(((1s,4s)-4-(dimethylamino)cyclohexyl)oxy)-3-methoxyphenyl)pyrimidin-2-amine

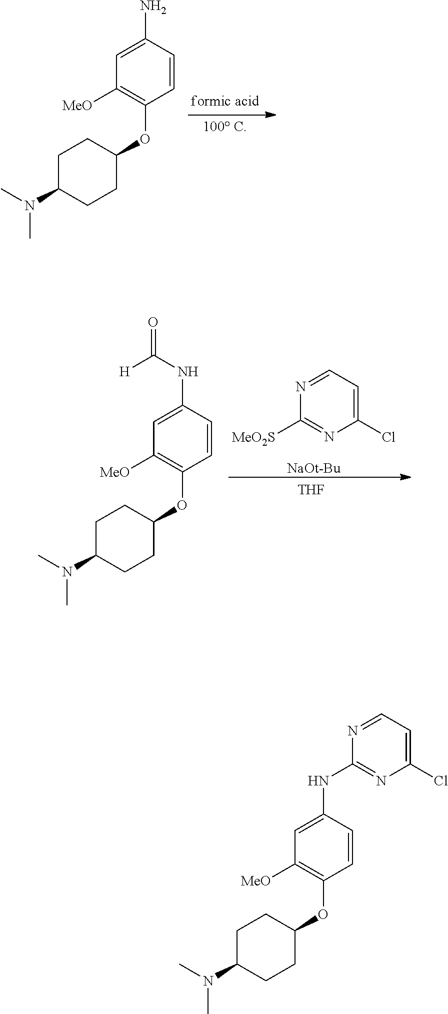 [SMILES: COc1cc(N)ccc1O[C@H]1CC[C@@H](N(C)C)CC1 ; COc1cc(NC=O)ccc1O[C@H]1CC[C@@H](N(C)C)CC1 ; CS(=O)(=O)c1nccc(Cl)n1 ; "THF" ; COc1cc(Nc2nccc(Cl)n2)ccc1O[C@H]1CC[C@@H](N(C)C)CC1 ; "NaOt-Bu"]

The title compound was prepared following general procedure A, starting from 4-(((1s,4s)-4-(dimethylamino)cyclohexyl)oxy)-3-methoxyaniline and 4-chloro-2-(methylsulfonyl)pyrimidine. MS (m/z): [M+H]⁺: 377.4.

### Example 90: Preparation of Compound 33

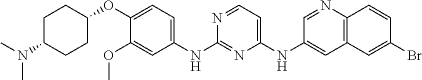 [SMILES: COc1cc(Nc2nccc(Nc3cnc4ccc(Br)cc4c3)n2)ccc1O[C@H]1CC[C@@H](N(C)C)CC1]

Compound 33 was prepared from the corresponding intermediate according to general procedure C. MS (m/z): [M+H]⁺ 563, 565.1. H NMR (500 MHz, DMSO): δ 9.94 (s, 1H), 9.10 (s, 1H), 9.03 (s, 1H), 8.94 (d, J=2.6 Hz, 1H), 8.11 (d, J=5.7 Hz, 1H), 8.05 (s, 1H), 7.87 (d, J=8.9 Hz, 1H), 7.69 (dd, J=8.9, 2.2 Hz, 1H), 7.33 (s, 1H), 7.26 (dd, J=8.6, 2.3 Hz, 1H), 6.90 (d, J=8.7 Hz, 1H), 6.32 (d, J=5.7 Hz, 1H), 4.38-4.31 (m, 1H), 3.69 (s, 3H), 2.19 (s, 6H), 2.17-2.10 (m, 1H), 1.98-1.86 (m, 2H), 1.75-1.63 (m, 2H), 1.57-1.46 (m, 4H).

### Example 91: Preparation of Compound 34

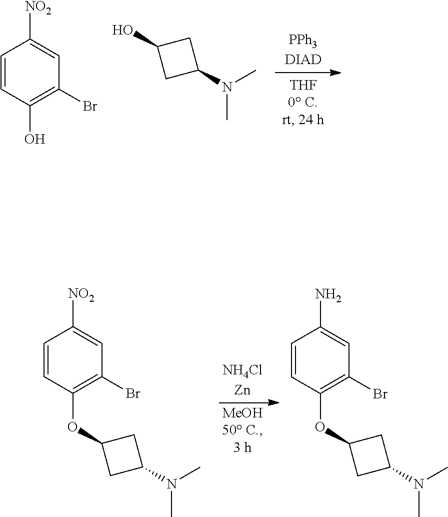

The title compound was prepared following general procedure T, starting from 2-bromo-4-nitrophenol and (1s,3s)-3-(dimethylamino)cyclobutan-1-ol in THF followed by nitro reduction employing the general nitro reduction procedure I. MS (m/z): [M+H]⁺: 285.1, 287.1.

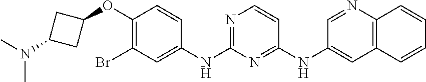 [SMILES: CN(C)[C@H]1C[C@H](Oc2ccc(Nc3nccc(Nc4cnc5ccccc5c4)n3)cc2Br)C1]

Compound 34 was prepared from the corresponding intermediate according to general procedure E. MS (m/z): [M+H]⁺ 505.7, 507.4. H NMR (400 MHz, DMSO): δ 9.85 (s, 1H), 9.23 (s, 1H), 8.94 (d, J=2.5 Hz, 1H), 8.91 (s, 1H), 8.10 (d, J=5.7 Hz, 1H), 7.96 (s, 1H), 7.93 (d, J=7.9 Hz, 1H), 7.80 (d, J=7.3 Hz, 1H), 7.66-7.52 (m, 3H), 6.82 (d, J=9.0 Hz, 1H), 6.33 (d, J=5.7 Hz, 1H), 4.79-4.70 (m, 1H), 2.91-2.81 (m, 1H), 2.38-2.30 (m, 2H), 2.20-2.12 (m, 2H), 2.08 (s, 6H).

### Example 92: Preparation of 3-chloro-4-((1r,3r)-3-(dimethylamino)cyclobutoxy)aniline (Intermediate 30)

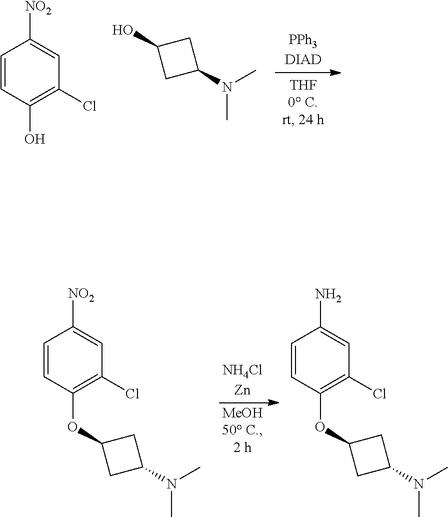

The title compound was prepared following general procedure T, starting from 2-chloro-4-nitrophenol and (1s,3s)-3-(dimethylamino)cyclobutan-1-ol in THF followed by nitro reduction employing the general nitro reduction procedure I.

### Example 93: Preparation of Compound 35

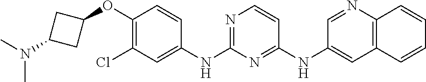 [SMILES: CN(C)[C@H]1C[C@H](Oc2ccc(Nc3nccc(Nc4cnc5ccccc5c4)n3)cc2Cl)C1]

Compound 35 was prepared from the corresponding intermediate according to general procedure E. MS (m/z): [M+H]⁺ 461.7, 463.5. H NMR (500 MHz, DMSO): δ (ppm): 9.86 (s, 1H), 9.24 (s, 1H), 8.94 (d, J=2.3 Hz, 1H), 8.90 (s, 1H), 8.23 (s, 1H, HCO₂H), 8.10 (d, J=5.7 Hz, 1H), 7.93 (d, J=8.3 Hz, 1H), 7.83 (s, 1H), 7.80 (d, J=8.1 Hz, 1H), 7.63-7.53 (m, 3H), 6.85 (d, J=9.0 Hz, 1H), 6.33 (d, J=5.7 Hz, 1H), 4.79-4.70 (m, 1H), 2.86 (tt, J=7.6, 5.4 Hz, 1H), 2.38-2.31 (m, 2H), 2.19-2.13 (m, 2H), 2.09 (s, 6H) (formate salt).

### Example 94: Preparation of 4-((1r,3r)-3-(dimethylamino)cyclobutoxy)-3-(trifluoromethyl)aniline (Intermediate 31)

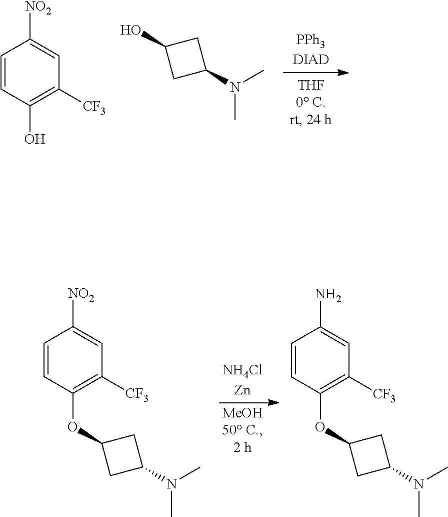

The title compound was prepared following general procedure T, starting from 4-nitro-2-(trifluoromethyl)phenol and (1s,3s)-3-(dimethylamino)cyclobutan-1-ol in THF followed by nitro reduction employing the general nitro reduction procedure I. MS (m/z): [M+H]⁺: 275.1.

### Example 95: Preparation of Compound 36

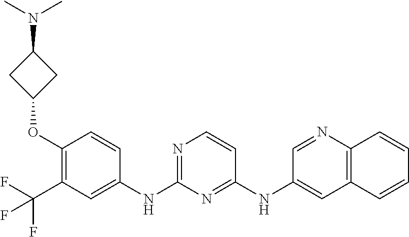 [SMILES: CN(C)[C@H]1C[C@H](Oc2ccc(Nc3nccc(Nc4cnc5ccccc5c4)n3)cc2C(F)(F)F)C1]

Compound 36 was prepared from the corresponding intermediate according to general procedure E. MS (m/z): [M+H]⁺ 495.3. ¹H NMR (500 MHz, DMSO): δ 9.86 (s, 1H), 9.33 (s, 1H), 8.95 (d, J=2.4 Hz, 1H), 8.89 (s, 1H), 8.11 (d, J=5.7 Hz, 1H), 7.97-7.91 (m, 3H), 7.80 (d, J=7.5 Hz, 1H), 7.60 (ddd, J=8.4, 6.9, 1.6 Hz, 1H), 7.58-7.53 (m, 1H), 6.94 (d, J=9.5 Hz, 1H), 6.34 (d, J=5.8 Hz, 1H), 4.86-4.79 (m, 1H), 2.86-2.78 (m, 1H), 2.38-2.32 (m, 2H), 2.17-2.10 (m, 2H), 2.08 (s, 6H).

### Example 96: Preparation of 3-methoxy-4-((1-methylazetidin-3-yl)oxy)aniline (Intermediate 32)

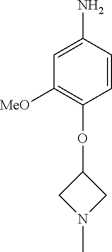

### Step 1: tert-butyl 3-(2-methoxy-4-nitrophenoxy)azetidine-1-carboxylate

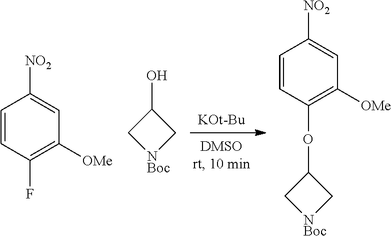 [SMILES: COc1cc([N+](=O)[O-])ccc1F ; CC(C)(C)OC(=O)N1CC(O)C1 ; COc1cc([N+](=O)[O-])ccc1OC1CN(C(=O)OC(C)(C)C)C1 ; "KOt-Bu"]

The title compound was prepared following general procedure G, starting from 1-fluoro-2-methoxy-4-nitrobenzene and Boc-protected azetidin-3-ol (tert-butyl 3-hydroxyazetidine-1-carboxylate) in DMSO.

### Step 2: 3-(2-methoxy-4-nitrophenoxy)-1-methylazetidine

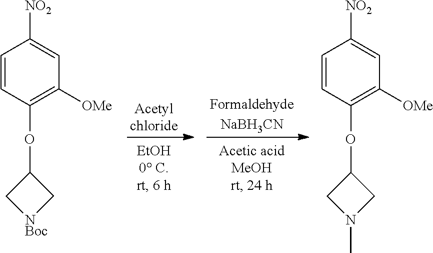 [SMILES: COc1cc([N+](=O)[O-])ccc1OC1CN(C(=O)OC(C)(C)C)C1 ; "Acetylchloride" ; "FormaldehydeNaBH3CN" ; COc1cc([N+](=O)[O-])ccc1OC1CN(C)C1]

### Step 2-1

To a solution of tert-butyl 3-(2-methoxy-4-nitrophenoxy)azetidine-1-carboxylate (900 mg, 2.77 mmol) in EtOH (10.0 mL) was added acetyl chloride (1.01 mL, 13.9 mmol) at 0° C. dropwise. Then the reaction was allowed to warm to room temperature and stirred for 6 hours. Solvent was removed under reduced pressure to give the free amine as a brown oil.

### Step 2-2

Into a solution of the product from Step 1 in MeOH (14.0 mL) was added formaldehyde (3.05 mmol), sodium cyanoborohydride (339 uL, 5.55 mmol) sodium cyanoborohydride (339 uL, 5.55 mmol) and acetic acid (794 uL, 13.9 mmol). After stirring for 24 h, the reaction was diluted with water, extracted with DCM (3×30 mL). The combined organics were dried, concentrated, and purified by column chromatography (DCM(1% triethylamine)/MeOH=0-10%) to give the title product (110 mg, 17%) as a brown solid. MS (m/z): [M+H]⁺: 239.

### Step 3: 3-methoxy-4-((1-methylazetidin-3-yl)oxy)aniline

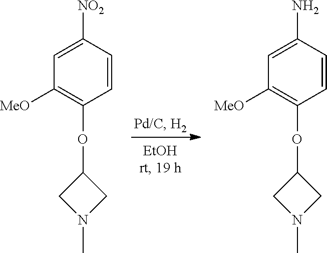

The title compound was prepared following general procedure H starting from 3-(2-methoxy-4-nitrophenoxy)-1-methylazetidine in EtOH. MS (m/z): [M+H]⁺: 209.

### Example 97: Preparation of Compound 37

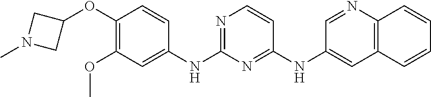 [SMILES: COc1cc(Nc2nccc(Nc3cnc4ccccc4c3)n2)ccc1OC1CN(C)C1]

Compound 37 was prepared from the corresponding intermediate according to general procedure E. MS (m/z): [M+H]⁺ 429.2. ¹H NMR (500 MHz, MeOD): δ 8.96 (d, J=2.4 Hz, 1H), 8.82 (d, J=2.6 Hz, 1H), 8.01 (d, J=5.8 Hz, 1H), 7.94 (d, J=8.6 Hz, 1H), 7.69-7.59 (m, 2H), 7.59-7.52 (m, 1H), 7.24 (d, J=2.4 Hz, 1H), 7.06 (dd, J=8.6, 2.4 Hz, 1H), 6.72 (d, J=8.6 Hz, 1H), 6.28 (d, J=5.8 Hz, 1H), 4.79-4.74 (m, 1H), 3.86-3.77 (m, 2H), 3.65 (s, 3H), 3.30-3.27 (m, 2H), 2.44 (s, 3H).

### Example 98: Preparation of 4-((1s,4s)-4-(4-amino-2-methoxyphenoxy)cyclohexyl)thiomorpholine 1,1-dioxide (Intermediate 33)

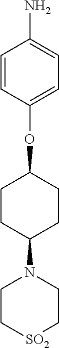 [SMILES: Nc1ccc(O[C@H]2CC[C@@H](N3CCS(=O)(=O)CC3)CC2)cc1]

### Step 1: 4-((1s,4s)-4-hydroxycyclohexyl)thiomorpholine 1,1-dioxide

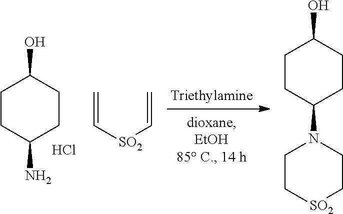

cis-4-Aminocyclohexanol hydrochloride (2.00 g, 13.2 mmol) was suspended in dioxane (15.0 mL) and EtOH (15.0 mL). Triethylamine (7.39 mL, 52.8 mmol) was added, followed by divinyl sulfone (2.65 mL, 26.4 mmol). The mixture was heated to 85° C. overnight. The mixture was concentrated to near dryness, charged with THF (30 mL), and the resulting slurry was filtered. The filtrate was concentrated to a solid which was triturated with ethyl ether to give the title compound (2.36 g, 69%) as a colorless solid. ¹H NMR (500 MHz, DMSO) δ (ppm): 4.28 (d, J=3.4 Hz, 1H), 3.77-3.71 (m, 1H), 3.05-3.00 (m, 4H), 2.96-2.90 (m, 4H), 2.49-2.42 (m, 1H), 1.70-1.56 (m, 4H), 1.44-1.32 (m, 4H).

### Step 2: 4-((1s,4s)-4-(4-amino-2-methoxyphenoxy)cyclohexyl)thiomorpholine 1,1-dioxide

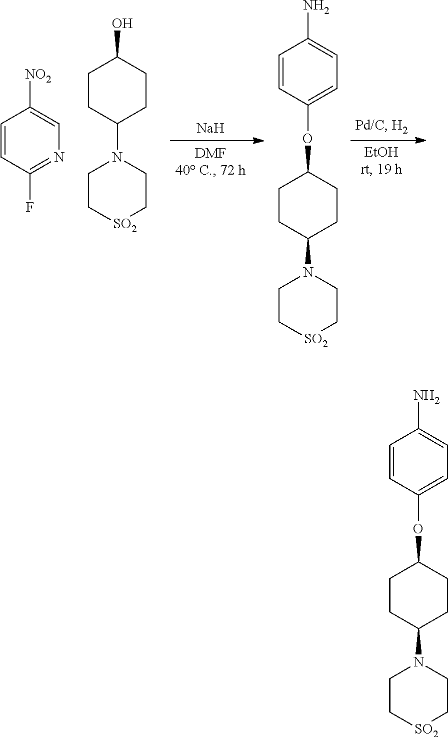

The title compound was prepared following general procedure G, starting from 4-((1s,4s)-4-hydroxycyclohexyl)thiomorpholine 1,1-dioxide and 1-fluoro-4-nitrobenzene in DMF followed by nitro reduction employing the general nitro reduction procedure H. MS (m/z): [M+H]⁺: 325.

### Example 99: Preparation of Compound 38

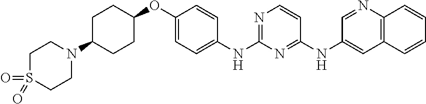 [SMILES: O=S1(=O)CCN([C@H]2CC[C@@H](Oc3ccc(Nc4nccc(Nc5cnc6ccccc6c5)n4)cc3)CC2)CC1]

Compound 38 was prepared from the corresponding intermediate according to general procedure E. MS (m/z): [M+H]⁺ 545.7. H NMR (500 MHz, DMSO): δ (ppm): 9.80 (s, 1H), 9.09 (s, 1H), 8.94 (s, 1H), 8.91 (d, J=2.2 Hz, 1H), 8.07 (d, J=5.7 Hz, 1H), 7.93 (d, J=8.4 Hz, 1H), 7.76 (d, J=5.8 Hz, 1H), 7.62-7.51 (m, 4H), 6.92-6.87 (m, 2H), 6.29 (d, J=5.7 Hz, 1H), 4.50-4.46 (m, 1H), 3.08-3.04 (m, 4H), 3.03-2.97 (m, 4H), 2.64 (tdd, J=6.8, 4.5, 2.4 Hz, 1H), 2.04-1.96 (m, 2H), 1.70-1.52 (m, 6H).

### Example 100: Preparation of 4-(((1s,4s)-4-morpholinocyclohexyl)oxyaniline (Intermediate 34)

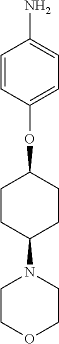 [SMILES: Nc1ccc(O[C@H]2CC[C@@H](N3CCOCC3)CC2)cc1]

### Step 1: (1s,4s)-4-morpholinocyclohexanol

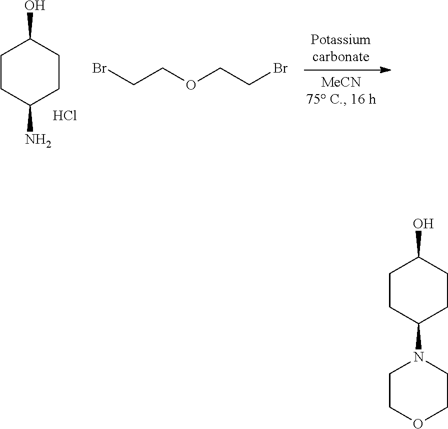 [SMILES: N[C@H]1CC[C@@H](O)CC1 ; Cl ; BrCCOCCBr ; O[C@H]1CC[C@@H](N2CCOCC2)CC1]

A mixture of cis-4-aminocyclohexanol hydrochloride (2.50 g, 16.2 mmol), 2-bromoethyl ether (3.17 mL, 24.2 mmol), and potassium carbonate (11.4 g, 80.8 mmol) in MeCN (80 mL) was stirred at 75° C. for 16 h. The reaction was filtered and concentrated under reduced pressure to give the title compound (3.05 g, 95%) as a white solid. ¹H NMR (500 MHz, DMSO) δ (ppm): 4.26 (d, J=3.5 Hz, 1H), 3.71-3.64 (m, 1H), 3.58-3.52 (m, 4H), 2.44-2.39 (m, 4H), 2.15-2.04 (m, 1H), 1.68-1.57 (m, 4H), 1.46-1.31 (m, 4H).

### Step 2: 4-(((1s,4s)-4-morpholinocyclohexyl)oxyaniline

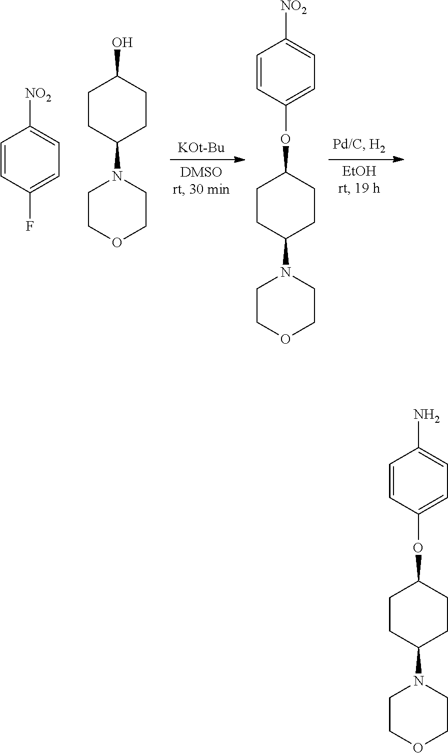

The title compound was prepared following general procedure G, starting from (1s,4s)-4-morpholinocyclohexanol and 1-fluoro-4-nitrobenzene in DMSO followed by nitro reduction employing the general nitro reduction procedure H. MS (m/z): [M+H]⁺: 277.

### Example 101: Preparation of Compound 39

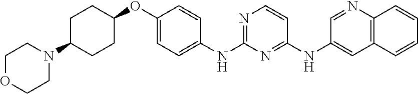 [SMILES: c1ccc2ncc(Nc3ccnc(Nc4ccc(O[C@H]5CC[C@@H](N6CCOCC6)CC5)cc4)n3)cc2c1]

Compound 39 was prepared from the corresponding intermediate according to general procedure E. MS (m/z): [M+H]⁺ 497.5. H NMR (500 MHz, DMSO): δ 9.80 (s, 1H), 9.08 (s, 1H), 8.95 (s, 1H), 8.90 (d, J=2.2 Hz, 1H), 8.07 (d, J=5.7 Hz, 1H), 7.93 (d, J=8.3 Hz, 1H), 7.76 (d, J=6.8 Hz, 1H), 7.59 (ddd, J=8.4, 6.9, 1.6 Hz, 1H), 7.57-7.52 (m, 3H), 6.91-6.86 (m, 2H), 6.28 (d, J=5.7 Hz, 1H), 4.45 (s, 1H), 3.62-3.55 (m, 4H), 2.49-2.45 (m, 4H), 2.27-2.18 (m, 1H), 2.00-1.89 (m, 2H), 1.70-1.53 (m, 6H).

### Example 102: Preparation of 4-(((1s,4s)-4-(dimethylamino)cyclohexyl)oxy)aniline (Intermediate 35)

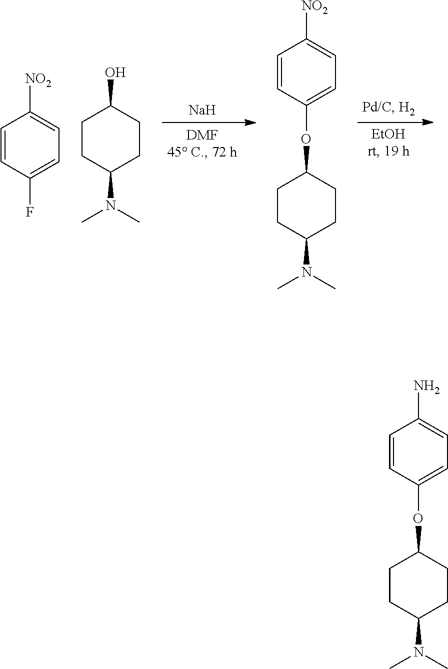

The title compound was prepared following general procedure G, starting from (1s,4s)-4-(dimethylamino)cyclohexanol and 1-fluoro-4-nitrobenzene in THF followed by nitro reduction employing the general nitro reduction procedure H. MS (m/z): [M+H]⁺: 235.

### Example 103: Preparation of Compound 40

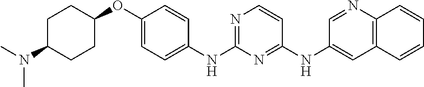 [SMILES: CN(C)[C@H]1CC[C@@H](Oc2ccc(Nc3nccc(Nc4cnc5ccccc5c4)n3)cc2)CC1]

Compound 40 was prepared from the corresponding intermediate according to general procedure E. MS (m/z): [M+H]⁺ 455.5. ¹H NMR (500 MHz, DMSO): δ 9.81 (s, 1H), 9.08 (s, 1H), 8.95 (s, 1H), 8.91 (d, J=2.5 Hz, 1H), 8.07 (d, J=5.7 Hz, 1H), 7.93 (d, J=8.3 Hz, 1H), 7.76 (d, J=7.7 Hz, 1H), 7.63-7.48 (m, 4H), 6.93-6.84 (m, 2H), 6.29 (d, J=5.7 Hz, 1H), 4.47-4.41 (m, 1H), 2.22 (s, 6H), 2.21-2.16 (m, 1H), 1.99-1.89 (m, 2H), 1.70-1.52 (m, 6H).

### Example 104: Preparation of 4-((1s,4s)-4-(4-aminophenoxy)cyclohexyl)piperazin-2-one (Intermediate 36)

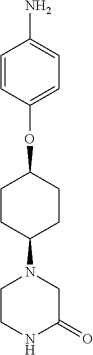 [SMILES: Nc1ccc(O[C@H]2CC[C@@H](N3CCNC(=O)C3)CC2)cc1]

### Step 1: 8-(4-nitrophenoxy)-1,4-dioxaspiro[4.5]decane

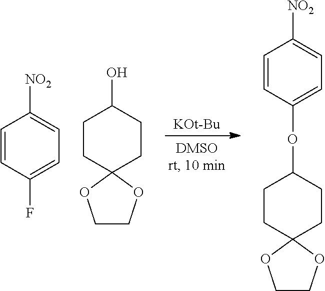

The title compound was prepared following general procedure G, starting from 1-fluoro-nitrobenzene and 1,4-dioxaspiro[4.5]decan-8-ol in DMSO.

### Step 2: 4-(4-nitrophenoxy)cyclohexanone

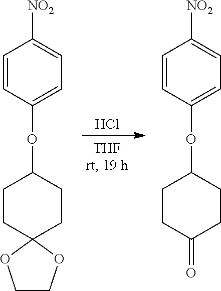 [SMILES: O=[N+]([O-])c1ccc(OC2CCC3(CC2)OCCO3)cc1 ; Cl ; O=C1CCC(Oc2ccc([N+](=O)[O-])cc2)CC1]

To a solution of 8-(4-nitrophenoxy)-1,4-dioxaspiro[4.5]decane (1.13 g, 4.05 mmol) in THF (16 mL) cooled to 0° C. was added a 1.0 M aqueous solution of hydrochloric acid (12 mL). The mixture was stirred at room temperature for 19 h and then concentrated under reduced pressure. The resulting solid was washed with hexane/ethyl acetate mixture (20:1) and dried under vacuum to give the title compound (800 mg, 84%).

### Step 3: 4-((1s,4s)-4-(4-aminophenoxy)cyclohexyl)piperazin-2-one

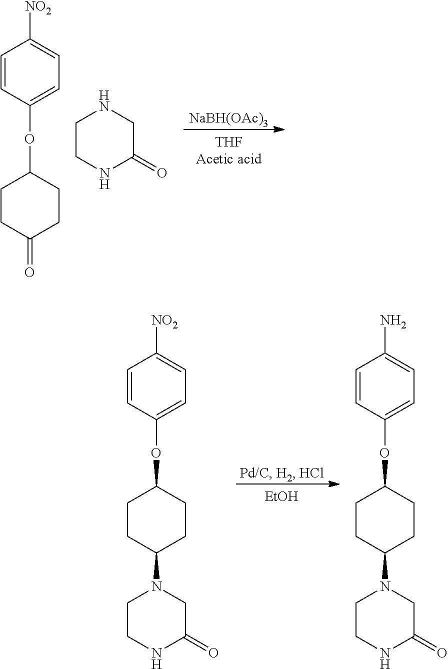

The title compound was prepared following general procedure U, starting from 4-(4-nitrophenoxy)cyclohexanone and 4-(4-nitrophenoxy)cyclohexanone. MS (m/z): [M+H]⁺: 290.

### Example 105: Preparation of Compound 41

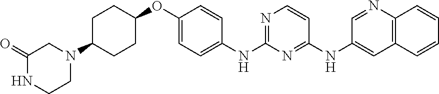 [SMILES: O=C1CN([C@H]2CC[C@@H](Oc3ccc(Nc4nccc(Nc5cnc6ccccc6c5)n4)cc3)CC2)CCN1]

Compound 41 was prepared from the corresponding intermediate according to general procedure E. MS (m/z): [M+H]⁺ 510.5. H NMR (500 MHz, DMSO): δ 9.80 (s, 1H), 9.08 (s, 1H), 8.95 (s, 1H), 8.91 (d, J=2.3 Hz, 1H), 8.07 (d, J=5.7 Hz, 1H), 7.93 (d, J=8.2 Hz, 1H), 7.76 (d, J=6.0 Hz, 1H), 7.72 (s, 1H), 7.61-7.51 (m, 4H), 6.92-6.87 (m, 2H), 6.29 (d, J=5.7 Hz, 1H), 4.48-4.43 (m, 1H), 3.17-3.12 (m, 2H), 3.05 (s, 2H), 2.68-2.63 (m, 2H), 2.36 (tt, J=7.7, 2.5 Hz, 1H), 1.99-1.91 (m, 2H), 1.72-1.53 (m, 6H).

### Example 106: Preparation of Compound 42

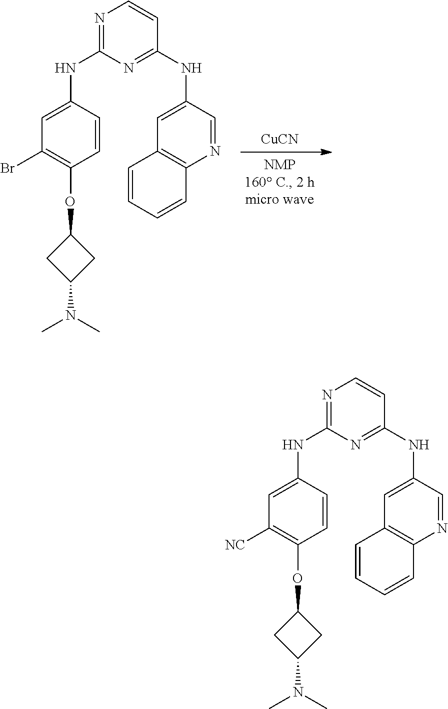

Compound 34 (30.0 mg, 59.4 umol) and Copper(I) cyanide (8.05 mg, 89.0 umol) were heated at reflux in NMP (500 uL) for 2 h under microwave irradiation. The crude was purified by column chromatography (SiO₂, gradient of Hexane in 30% IPA in EtOAc with 10% NH₄OH) to give the desired product, which was triturated with EtOAc/hexane to purify further, affording the title compound (1.30 mg, 4.9%), a white solid. MS (m/z): [M+H]⁺ 452.2. ¹H NMR (500 MHz, DMSO): δ 9.88 (s, 1H), 9.36 (s, 1H), 8.97 (s, 1H), 8.86 (s, 1H), 8.13 (d, J=5.4 Hz, 1H), 8.06 (d, J=4.2 Hz, 1H), 7.94 (d, J=8.0 Hz, 1H), 7.89 (dd, J=9.1, 2.7 Hz, 1H), 7.81 (dd, J=7.6, 3.7 Hz, 1H), 7.63-7.54 (m, 2H), 6.96 (d, J=8.7 Hz, 1H), 6.36 (d, J=5.8 Hz, 1H), 4.87-4.80 (m, 1H), 2.93-2.82 (m, 1H), 2.42-2.38 (m, 2H), 2.21-2.15 (m, 2H), 2.10 (s, 6H).

### Example 107: Preparation of Compound 43

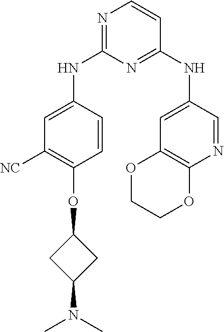 [SMILES: CN(C)[C@H]1C[C@@H](Oc2ccc(Nc3nccc(Nc4cnc5c(c4)OCCO5)n3)cc2C#N)C1]

### Step 1: 3-bromo-4-((1s,3s)-3-(dimethylamino)cyclobutoxy)aniline

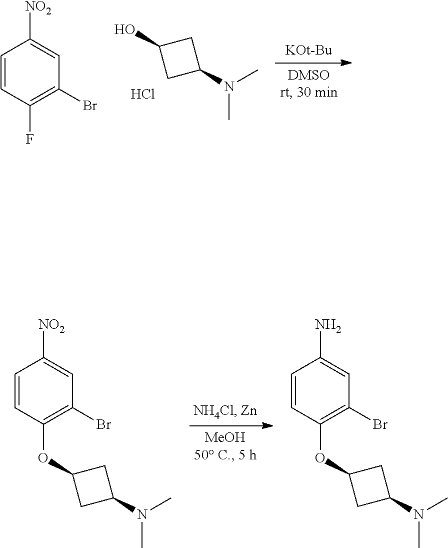

The title compound was prepared following general procedure G, starting from 2-bromo-1-fluoro-4-nitrobenzene and (1s,3s)-3-(dimethylamino)cyclobutan-1-ol HCl salt in DMSO followed by nitro reduction employing the general nitro reduction procedure I. MS (m/z): [M+H]⁺: 285.1, 287.1

### Step 2: N-(2-chloropyrimidin-4-yl)-2,3-dihydro-[1,4]dioxino[2,3-b]pyridin-7-amine

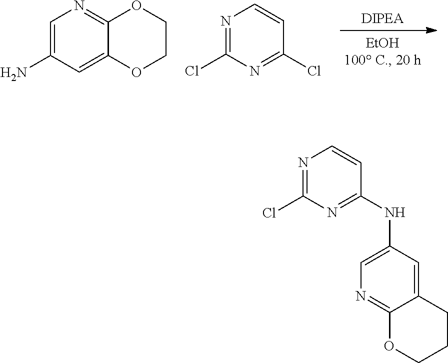

The title compound was prepared following general procedure B, starting 2,4-dichloropyrimidine and 2,3-dihydro-[1,4]dioxino[2,3-b]pyridin-7-amine in EtOH. MS (m/z): [M+H]⁺: 263.0.

### Step 3: 5-bromo-N2-(3-methoxy-4-morpholinophenyl)-N4-(quinolin-3-yl)pyrimidine-2,4-diamine

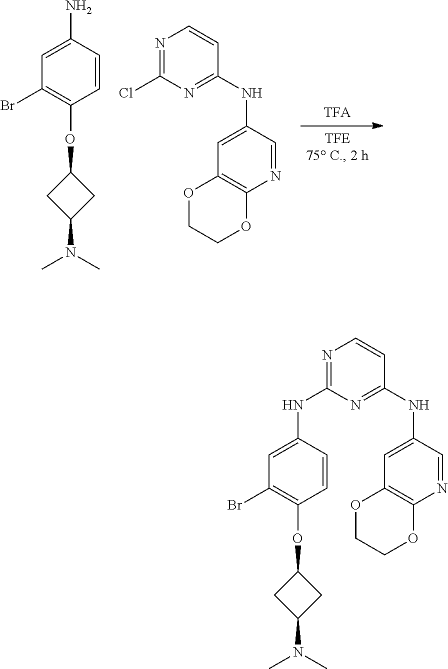

The title compound was prepared following general procedure E, starting from N-(2-chloropyrimidin-4-yl)-2,3-dihydro-[1,4]dioxino[2,3-b]pyridin-7-amine and 3-bromo-4-((1s,3s)-3-(dimethylamino)cyclobutoxy)aniline in TFE. MS (m/z): [M+H]⁺: 513.2, 515.2.

### Step 4

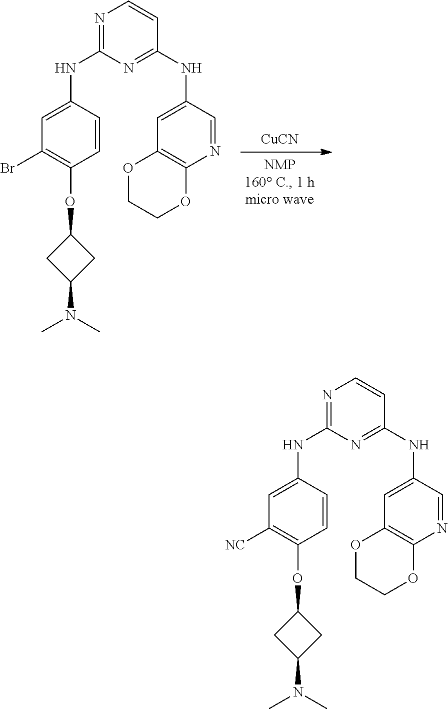

5-bromo-N2-(3-methoxy-4-morpholinophenyl)-N4-(quinolin-3-yl)pyrimidine-2,4-diamine (30.0 mg, 58.4 umol) and Copper(I) cyanide (10.6 mg, 117 umol) were heated at reflux in NMP (500 uL) for 1 h under microwave irradiation. The crude was purified by interative chromatography (C18, gradient of 10 mM ammonium bicarbonate buffer in MeCN) to give the title compound (2.43 mg, 9.0%) as a white solid. MS (m/z): [M+H]⁺ 460.5. ¹H NMR (500 MHz, MeOD): δ 7.86-7.81 (m, 2H), 7.68 (s, 2H), 7.53 (d, J=11.0 Hz, 1H), 6.87 (d, J=8.6 Hz, 1H), 6.06 (d, J=5.9 Hz, 1H), 4.51-4.48 (m, 1H), 4.36-4.32 (m, 2H), 4.22-4.16 (m, 2H), 2.73-2.65 (m, 2H), 2.50-2.42 (m, 1H), 2.11 (s, 6H), 2.00-1.94 (m, 2H).

### Example 108: Preparation of 5-amino-2-((1r,3r)-3-(dimethylamino)cyclobutoxy)-3-methoxybenzonitrile

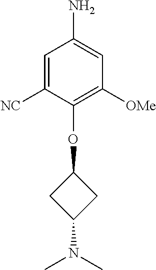 [SMILES: COc1cc(N)cc(C#N)c1O[C@H]1C[C@H](N(C)C)C1]

### Step 1: 2-hydroxy-3-methoxy-5-nitrobenzonitrile

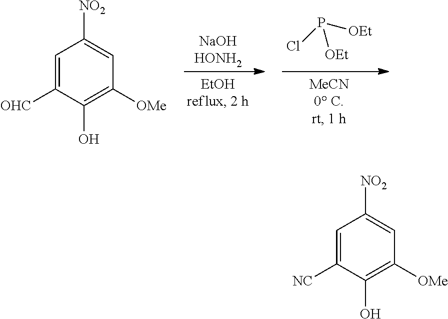 [SMILES: COc1cc([N+](=O)[O-])cc(C=O)c1O ; CCOP(Cl)OCC ; COc1cc([N+](=O)[O-])cc(C#N)c1O]

### Step 1.1

A mixture of hydroxylamine hydrochloride (7.19 g, 101 mmol) and sodium Hydroxide (4.06 g, 101 mmol) in EtOH (40 mL) was warmed to 70° C. A solution of 3-methoxy-5-nitrosalicylaldehyde (5.00 g, 25.4 mmol) in EtOH (40 mL) was added. After the resulting mixture was heated under reflux for 2 h, the solvent was removed under vacuum. The residue was partitioned between 300 mL of ether and 500 mL of water. The organic phase was washed three times with water and dried over Na₂SO₄. After evaporation of the solvent and drying of the compound in a vacuum, 2-hydroxy-3-methoxy-5-nitrobenzaldehyde oxime was obtained as a yellow solid. MS (m/z): [M+H]⁺: 213.1.

### Step 1.2

A solution of 2-hydroxy-3-methoxy-5-nitrobenzaldehyde oxime (500 mg, 2.36 mmol) in MeCN (20 mL) was cooled to 0° C. Diethyl chlorophosphite (706 uL, 4.71 mmol) was added to the solution dropwise at the same temperature. After completion of the addition, the reaction was warmed to rt and stirred for 1 hour. 2-hydroxy-3-methoxy-5-nitrobenzonitrile was obtained as a white solid (150 mg, 33% over 2 steps). MS (m/z): [M−H]⁻: 193.1.

### Step 2: 5-amino-2-((1r,3r)-3-(dimethylamino)cyclobutoxy)-3-methoxybenzonitrile

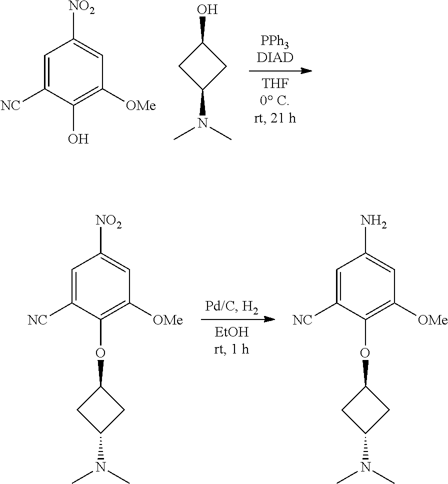

The title compound was prepared following general procedure T, starting from 2-hydroxy-3-methoxy-5-nitrobenzonitrile and (1s,3s)-3-(dimethylamino)cyclobutan-1-ol in THF followed by nitro reduction employing the general nitro reduction procedure H. MS (m/z): [M+H]⁺: 262.

### Example 109: Preparation of Compound 44

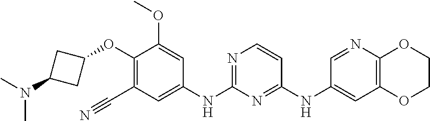 [SMILES: COc1cc(Nc2nccc(Nc3cnc4c(c3)OCCO4)n2)cc(C#N)c1O[C@H]1C[C@H](N(C)C)C1]

Compound 44 was prepared from intermediate 5-amino-2-((1r,3r)-3-(dimethylamino)cyclobutoxy)-3-methoxybenzonitrile according to general procedure E. MS (m/z): [M+H]⁺ 490.3. ¹H NMR (500 MHz, DMSO): δ 9.42 (s, 1H), 9.37 (s, 1H), 8.05 (d, J=5.8 Hz, 1H), 7.94 (s, 1H), 7.77-7.73 (m, 2H), 7.60 (d, J=2.1 Hz, 1H), 6.21 (d, J=5.8 Hz, 1H), 4.85-4.77 (m, 1H), 4.39-4.34 (m, 2H), 4.27-4.22 (m, 2H), 3.75 (s, 3H), 2.94-2.87 (m, 1H), 2.20-2.08 (m, 4H), 2.04 (s, 6H).

### Example 110: Preparation of 1-methyl-3′H-spiro[azetidine-3,2′-benzofuran]-6′-amine HCl salt (Intermediate 37)

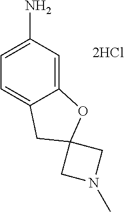

### Step 1: (6-bromo-2,3-dihydrobenzofuran-2,2-diyl)dimethanol

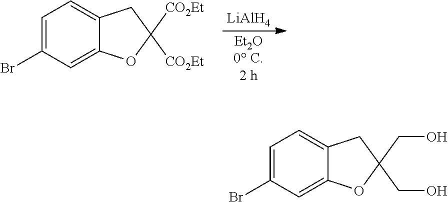 [SMILES: CCOC(=O)C1(C(=O)OCC)Cc2ccc(Br)cc2O1 ; "Et2O0° C.2 h" ; "LiAlH4" ; OCC1(CO)Cc2ccc(Br)cc2O1]

A solution of 2M lithium aluminum hydride in THF (11.7 mL, 23.3 mmol) was added dropwise to a solution of diethyl 6-bromobenzofuran-2,2(3H)-dicarboxylate (1.60 g, 4.66 mmol) in ether (50.0 mL) at 0° C. over 20 min. The reaction was stirred for 2 h at the same temperature. The reaction mixture was quenched with water (0.88 mL), 15% NaOH aq. (0.88 mL), and water (0.88 mL×3) at 0° C. Sodium sulfate was added to the suspension and the mixture was stirred for 30 min. The obtained suspension was filtered and washed with ethyl acetate. The filtrate was concentrated under reduced pressure to give the title product (890 mg, 44%).

### Step 2: 1-methyl-3′H-spiro[azetidine-3,2′-benzofuran]

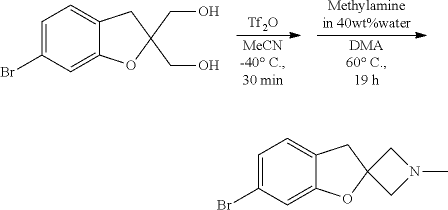

### Step 2.1

To a solution of (6-bromo-2,3-dihydrobenzofuran-2,2-diyl)dimethanol (1.30 g, 3.01 mmol) in MeCN (4 mL) was added trifluoromethanesulfonic anhydride (1.06 mL, 6.32 mmol) followed by N,N-diisopropylethylamine (1.32 mL, 7.53 mmol) dropwise at −40° C. After stirring for 30 min, the reaction was quenched with brine and extracted with ethyl acetate three times. The combined organic phase was dried over Na₂SO₄, filtered, and concentrated under reduced pressure to give a crude intermediate. MS (m/z):

### Step 2.2

The crude intermediate was dissolved in DMA (6 mL). Then methylamine in water (40 wt %, 576 μL, 6.66 mmol) was added to the solution. After stirring the reaction for 19 hours at 60° C., the reaction was poured into water and extracted with ethyl acetate three times. The combined organic phase was washed with 10% LiCl and brine, dried over Na₂SO₄, filtered, and concentrated under reduced pressure. The crude was purified by iterative chromatography (SiO₂, gradient of Hexane in 30% IPA in EtOAc (10% NH4OH) or C18, 10 mM ammonium bicarbonate buffer in MeCN) to give the title compound (100 mg, 17%) as a brown oil. MS (m/z): [M+H]⁺: 254.0, 256.0.

### Step 3: 1-methyl-3′H-spiro[azetidine-3,2′-benzofuran]-6′-amine HCl salt

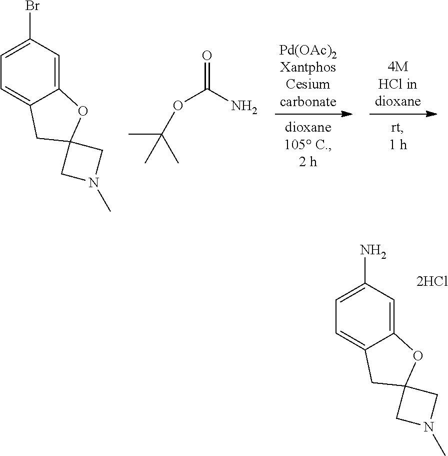

The title compound was prepared following general procedure L, starting from 1-methyl-3′H-spiro[azetidine-3,2′-benzofuran]followed by Boc-deprotection with HCl employing procedure M. MS (m/z): [M+H]⁺: 291.3 (NBoc intermediate).

### Example 111: Preparation of Compound 45

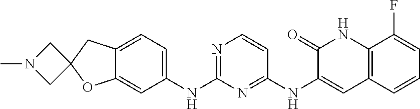 [SMILES: CN1CC2(Cc3ccc(Nc4nccc(Nc5cc6cccc(F)c6[nH]c5=O)n4)cc3O2)C1]

Compound 45 was prepared from the corresponding intermediate according to general procedure E. MS (m/z): [M+H]⁺ 445.5. ¹H NMR (500 MHz, DMSO): δ 12.29 (s, 1H), 9.26 (s, 1H), 9.08 (s, 1H), 9.01 (s, 1H), 8.10 (d, J=5.7 Hz, 1H), 7.35 (s, 2H), 7.29-7.24 (m, 1H), 7.19 (dd, J=10.5, 5.4 Hz, 1H), 7.11 (d, J=8.0 Hz, 1H), 7.07-7.01 (m, 1H), 6.80 (d, J=5.7 Hz, 1H), 3.46 (d, J=7.7 Hz, 2H), 3.39 (s, 2H), 3.24 (d, J=8.3 Hz, 2H), 2.30 (s, 3H).

### Example 112: Preparation of 5-amino-2-((1s,3s)-3-(dimethylamino)cyclobutoxy)nicotinonitrile (Intermediate 38)

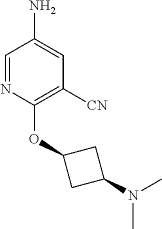 [SMILES: CN(C)[C@H]1C[C@@H](Oc2ncc(N)cc2C#N)C1]

### Step 1: (1s,3s)-3-((3-bromo-5-nitropyridin-2-yl)oxy)-N,N-dimethylcyclobutanamine

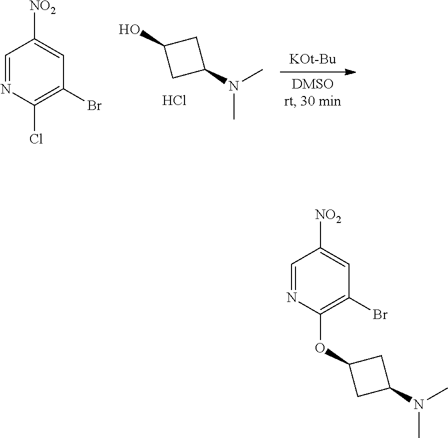

The title compound was prepared following general procedure G, starting from 3-bromo-2-chloro-5-nitropyridine and (1s,3s)-3-(dimethylamino)cyclobutan-1-ol HCl salt in DMSO. MS (m/z): [M+H]⁺: 316.0, 318.0.

### Step 2: 2-((1s,3s)-3-(dimethylamino)cyclobutoxy)-5-nitronicotinonitrile

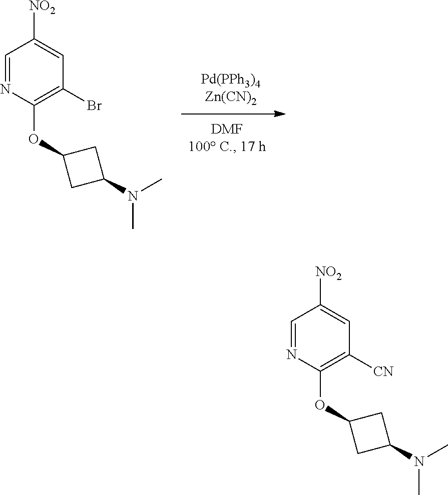 [SMILES: CN(C)[C@H]1C[C@@H](Oc2ncc([N+](=O)[O-])cc2Br)C1 ; CN(C)[C@H]1C[C@@H](Oc2ncc([N+](=O)[O-])cc2C#N)C1]

To a solution of (1s,3s)-3-((3-bromo-5-nitropyridin-2-yl)oxy)-N,N-dimethylcyclobutanamine (60.0 mg, 190 umol) and zinc cyanide (18.2 mg, 152 umol) in DMF (450 uL) was added tetrakis(triphenylphosphine)palladium(0) (22.2 mg, 19.0 umol). The reaction was heated at 100° C. for 17 hours. The reaction mixture was purified by column chromatography (10 mM ammonium bicarbonate buffer/MeCN=30-50%) to give the title compound (30.0 mg, 60%) as a brown solid. MS (m/z): [M+H]⁺: 263.2.

### Step 3: 5-amino-2-((1s,3s)-3-(dimethylamino)cyclobutoxy)nicotinonitrile

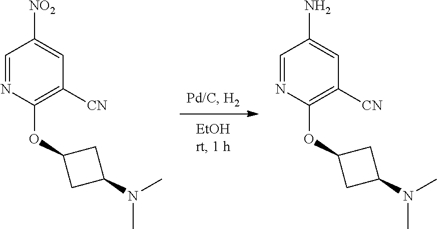 [SMILES: CN(C)[C@H]1C[C@@H](Oc2ncc([N+](=O)[O-])cc2C#N)C1 ; CN(C)[C@H]1C[C@@H](Oc2ncc(N)cc2C#N)C1]

The title compound was prepared following nitro reduction procedure H in EtOH. MS (m/z): [M+H]⁺: 233.

### Example 113: Preparation of Compound 46

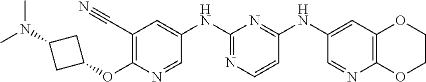 [SMILES: CN(C)[C@H]1C[C@@H](Oc2ncc(Nc3nccc(Nc4cnc5c(c4)OCCO5)n3)cc2C#N)C1]

Compound 46 was prepared from the corresponding intermediate according to general procedure E. MS (m/z): [M+H]⁺ 461.3. ¹H NMR (400 MHz, dmso): δ 9.44 (s, 1H), 9.41 (s, 1H), 8.55 (s, 2H), 8.02 (d, J=5.8 Hz, 1H), 7.89 (s, 1H), 7.70 (s, 1H), 6.20 (d, J=5.8 Hz, 1H), 4.94 (p, J=7.5 Hz, 1H), 4.40-4.35 (m, 2H), 4.28-4.22 (m, 2H), 2.65-2.58 (m, 2H), 2.41-2.33 (m, 1H), 2.05 (s, 6H), 1.92-1.82 (m, 2H).

### Example 114: Preparation of 3-methoxy-4-((1s,3s)-3-((2-methoxyethyl)(methyl)amino)cyclobutoxy)aniline (Intermediate 39)

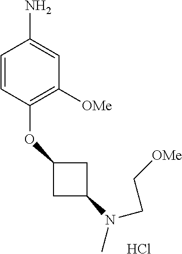 [SMILES: COCCN(C)[C@H]1C[C@@H](Oc2ccc(N)cc2OC)C1 ; Cl]

### Step 1: (1s,3s)-3-((2-methoxyethyl)(methyl)amino)cyclobutanol HCl salt

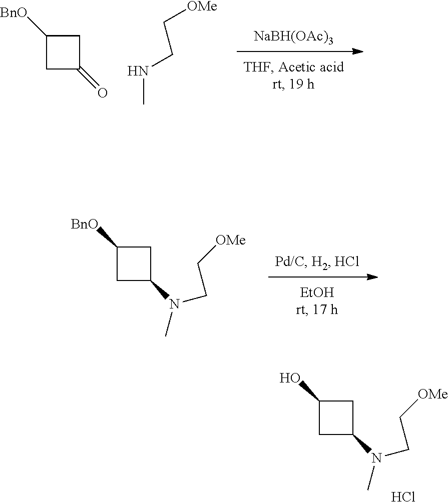

The title compound was prepared following general procedure U without basification, starting from 2-methoxy-N-methylethan-1-amine. MS (m/z): [M+H]⁺: 160.1.

### Step 2: 3-methoxy-4-((1s,3s)-3-((2-methoxyethyl)(methyl)amino)cyclobutoxy)aniline

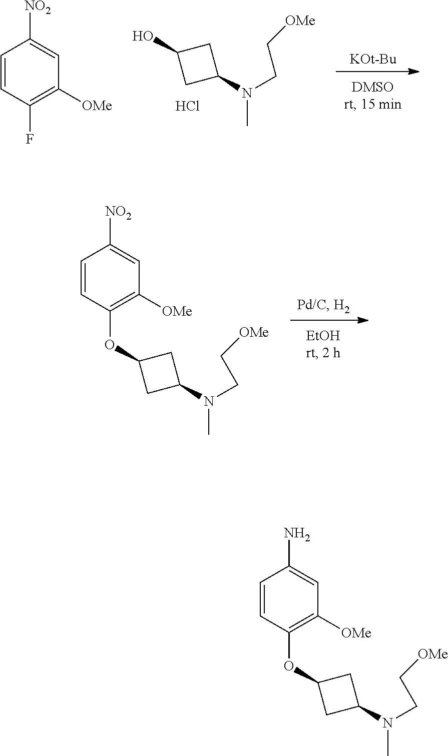

The title compound was prepared following general procedure G, starting from 1-fluoro-2-methoxy-4-nitrobenzene and (1s,3s)-3-((2-methoxyethyl)(methyl)amino)cyclobutanol HCl salt in DMSO followed by nitro reduction employing the general nitro reduction procedure I. MS (m/z): [M+H]⁺: 281.

### Example 115: Preparation of Compound 47

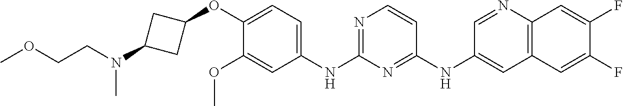 [SMILES: COCCN(C)[C@H]1C[C@@H](Oc2ccc(Nc3nccc(Nc4cnc5cc(F)c(F)cc5c4)n3)cc2OC)C1]

Compound 47 was prepared from the corresponding intermediate according to general procedure E. MS (m/z): [M+H]⁺ 537.4. H NMR (500 MHz, DMSO): δ 9.90 (s, 1H), 9.05 (s, 1H), 8.97 (s, 1H), 8.95 (s, 1H), 8.09 (d, J=5.7 Hz, 1H), 7.97-7.91 (m, 1H), 7.67 (s, 1H), 7.25 (s, 1H), 7.20 (d, J=8.3 Hz, 1H), 6.77 (d, J=8.7 Hz, 1H), 6.29 (d, J=5.7 Hz, 1H), 4.34 (p, J=7.2 Hz, 1H), 3.65 (s, 3H), 3.39 (t, J=6.0 Hz, 2H), 3.23 (s, 3H), 2.62-2.55 (m, 2H), 2.55-2.51 (m, 1H), 2.40 (t, J=6.0 Hz, 2H), 2.09 (s, 3H), 1.86-1.78 (m, 2H).

### Example 116: Preparation of 6-(3-(dimethylamino)azetidin-1-yl)-5-methoxypyridin-3-amine (Intermediate 40)

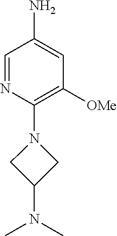 [SMILES: COc1cc(N)cnc1N1CC(N(C)C)C1]

### Step 1: N,N-dimethylazetidin-3-amine HCl salt

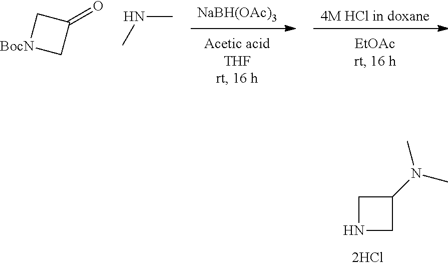

### Step 1.1

1-Boc-3-azetidinone (5.00 g, 28.6 mmol) was dissolved in THF (55 mL) and dimethylamine (5.44 mL, 42.9 mmol) (40% in water) was added in one portion. Acetic acid (5.65 mL, 98.7 mmol) was added. The reaction was stirred for 30 min at rt. Sodium triacetoxyborohydride (10.6 g, 48.7 mmol) was added. The reaction was left to stir at rt overnight. The reaction was quenched by slow addition of NaOH 3 M aq. (60 mL). EtOAc (60 mL) was added and the layers were separated. The aqueous layer was basified to pH 10 with NaOH 50% aq. and extracted with EtOAc (2×60 mL). The combined organic layers were washed with brine (30 mL), dried over Na2SO4 and concentrated. The crude was purified by column chromatography (Hexane/30% IPA in EtOAc (10% NH₄OH)=0-90%) to give tert-butyl 3-(dimethylamino)azetidine-1-carboxylate (2.10 g, 24%) as a mixture of alcohol derived from the reduction of starting material (˜2:1 ratio by ¹H NMR).

### Step 1.2

The obtained material was dissolved in EtOAc (11 mL) and 4M HCl in doxane (17 mL). The solution was stirred for 16 h. The reaction was concentrated under reduced pressure to give the title product (1.40 g, 18%) as a sticky white solid. The product was used for a next step without further purification.

### Step 2: 6-(3-(dimethylamino)azetidin-1-yl)-5-methoxypyridin-3-amine

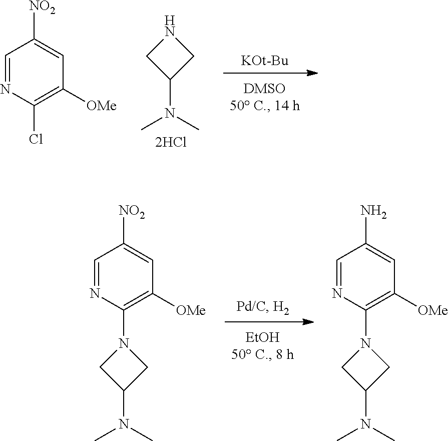

The title compound was prepared following general procedure G, starting from 2-chloro-3-methoxy-5-nitropyridine and N,N-dimethylazetidin-3-amine HCl salt in DMSO followed by nitro reduction employing the general nitro reduction procedure I. MS (m/z): [M+H]⁺: 223.1.

### Example 117: Preparation of Compound 48

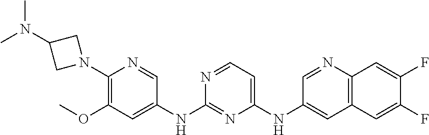 [SMILES: COc1cc(Nc2nccc(Nc3cnc4cc(F)c(F)cc4c3)n2)cnc1N1CC(N(C)C)C1]

Compound 48 was prepared from the corresponding intermediate according to general procedure F. MS (m/z): [M+H]⁺ 479.5. H NMR (500 MHz, DMSO): δ 9.87 (s, 1H), 8.96 (s, 1H), 8.93 (s, 1H), 8.81 (s, 1H), 8.01 (d, J=5.6 Hz, 1H), 7.98 (d, J=2.0 Hz, 1H), 7.86 (dd, J=11.5, 8.0 Hz, 1H), 7.76-7.68 (m, 1H), 7.27 (s, 1H), 6.22 (d, J=5.7 Hz, 1H), 3.99 (t, J=7.5 Hz, 2H), 3.75-3.69 (m, 2H), 3.57 (s, 3H), 3.02 (p, J=6.1 Hz, 1H), 2.03 (s, 6H).

### Example 118: Preparation of 2-(4-(5-amino-3-methoxypyridin-2-yl)piperazin-1-yl)acetonitrile (Intermediate 41)

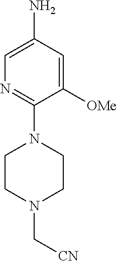 [SMILES: COc1cc(N)cnc1N1CCN(CC#N)CC1]

### Step 1: 1-(3-methoxy-5-nitropyridin-2-yl)piperazine

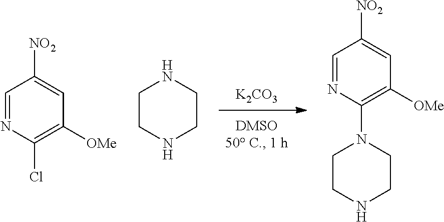

The title compound was prepared following general procedure G, starting from 3-methoxy-2-chloro-5-nitropyridine and piperazine in DMSO. MS (m/z): [M+H]⁺: 239.

### Step 2: 2-(4-(3-methoxy-5-nitropyridin-2-yl)piperazin-1-yl)acetonitrile

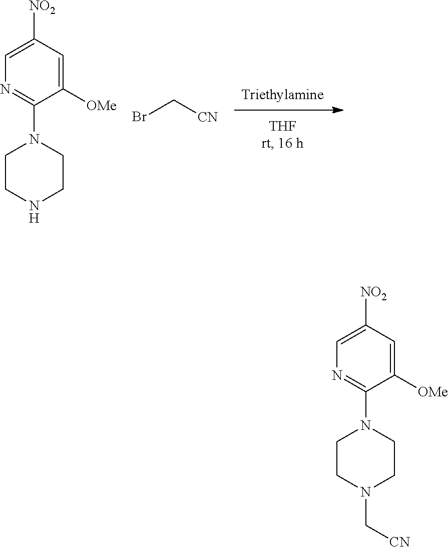

1-(3-methoxy-5-nitropyridin-2-yl)piperazine (375 mg, 1.57 mmol) was dissolved in THF (6 mL). Triethylamine (665 uL, 4.72 mmol) was added, followed by bromoacetonitrile (164 uL, 2.36 mmol). The reaction was stirred overnight. The precipitate was filtered and washed with THF (5 mL×2), and the obtained filtrate was dried under reduced pressure to give the title product (330 mg, 79%) as a yellow solid. MS (m/z): [M+H]⁺: 278.

### Step 3: 2-(4-(5-amino-3-methoxypyridin-2-yl)piperazin-1-yl)acetonitrile

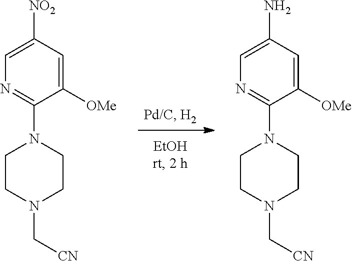 [SMILES: COc1cc([N+](=O)[O-])cnc1N1CCN(CC#N)CC1 ; "Pd/C, H2" ; COc1cc(N)cnc1N1CCN(CC#N)CC1]

The title compound was prepared following general procedure H, starting from 2-(4-(3-methoxy-5-nitropyridin-2-yl)piperazin-1-yl)acetonitrile in EtOH. MS (m/z): [M+H]⁺: 248.

### Example 119: Preparation of Compound 49

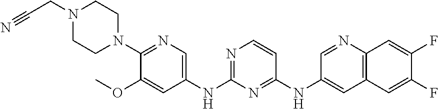 [SMILES: COc1cc(Nc2nccc(Nc3cnc4cc(F)c(F)cc4c3)n2)cnc1N1CCN(CC#N)CC1]

Compound 49 was prepared from the corresponding intermediate according to general procedure F. MS (m/z): [M+H]⁺ 504.0. ¹H NMR (500 MHz, DMSO): δ 9.97 (s, 1H), 9.16 (s, 1H), 9.02 (s, 1H), 8.91 (s, 1H), 8.20 (d, J=2.1 Hz, 1H), 8.11 (d, J=5.7 Hz, 1H), 7.93 (dd, J=11.5, 8.0 Hz, 1H), 7.74 (s, 1H), 7.52 (s, 1H), 6.33 (d, J=5.7 Hz, 1H), 3.80 (s, 2H), 3.69 (s, 3H), 3.31-3.26 (m, 4H), 2.64 (t, J=4.7 Hz, 4H).

### Example 120: Preparation of 5-methoxy-6-(2-oxa-6-azaspiro[3.3]heptan-6-yl)pyridin-3-amine

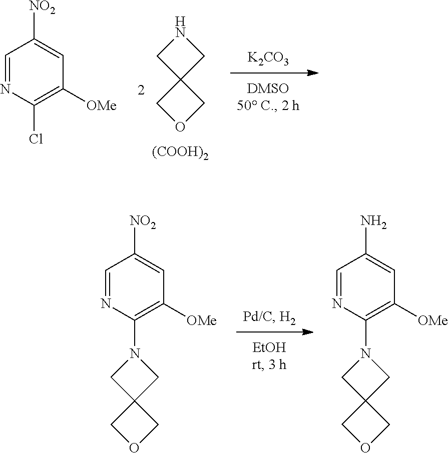

The title compound was prepared following general procedure G, starting from 2-chloro-3-methoxy-5-nitropyridine and 2-oxa-6-azaspiro[3.3]heptane oxalate salt in DMSO followed by nitro reduction employing the general nitro reduction procedure I. MS (m/z): [M+H]⁺: 222.

### Example 121: Preparation of Compound 50

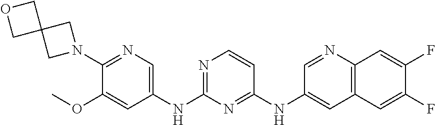 [SMILES: COc1cc(Nc2nccc(Nc3cnc4cc(F)c(F)cc4c3)n2)cnc1N1CC2(COC2)C1]

Compound 50 was prepared from the corresponding intermediate according to general procedure F. MS (m/z): [M+H]⁺ 478.4. ¹H NMR (500 MHz, DMSO): δ 10.03 (s, 1H), 8.99 (s, 2H), 8.89 (s, 1H), 8.06 (d, J=5.7 Hz, 1H), 8.03 (d, J=2.0 Hz, 1H), 7.91 (dd, J=11.5, 8.2 Hz, 1H), 7.71 (s, 1H), 7.34 (s, 1H), 6.29 (d, J=5.6 Hz, 1H), 4.72 (s, 4H), 4.16 (s, 4H), 3.63 (s, 3H).

### Example 122: Preparation of 4-(3,3-difluoropiperidin-4-yl)-3-methoxyaniline (Intermediate 43)

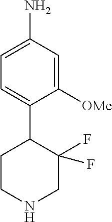 [SMILES: COc1cc(N)ccc1C1CCNCC1(F)F]

### Step 1: benzyl 4-(2-methoxy-4-nitrophenyl)-5,6-dihydropyridine-1(2H)-carboxylate

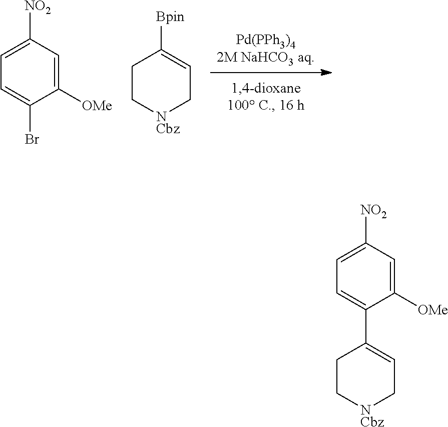

A solution of 2-Bromo-5-nitroanisole (3.00 g, 12.9 mmol), N-cbz-1,2,3,6-tetrahydropyridine-4-boronic acid pinacol ester (4.66 g, 13.6 mmol), tetrakis(triphenylphosphine)palladium(0) (755 mg, 646 umol), sodium hydrogenocarbonate (26.4 mL, 52.9 mmol) in dioxane (65 mL) was sealed and heated to 100° C. for 16 hours with stirring. After cooling to rt, dioxane was removed under reduced pressure. Then the residue was diluted with water and extracted with ethyl acetate three times. The combined organic phase was dried over Na₂SO₄, filtered, and concentrated under reduced pressure to give a brown oil. The oil was dissolved in EtOAc/Heptane and the filtrate was concentrated and purified by column chromatography (Hexane/EtOAc=10-50%) to give the title compound (3.00 g, 63%) as an yellow oil. MS (m/z): [M+H]⁺: 369.

### Step 2: benzyl 3,3-difluoro-4-(2-methoxy-4-nitrophenyl)piperidine-1-carboxylate

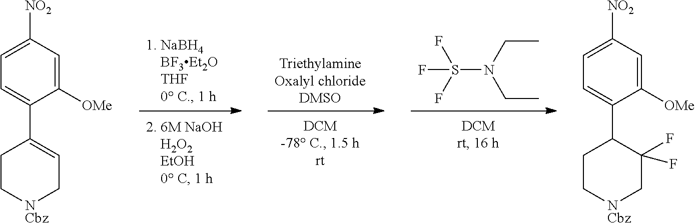

### Step 2.1

To a stirring mixture of sodium borohydride (366 mg, 9.28 mmol) in THF (25 mL) cooled to 0° C., was added boron trifluoride diethyl etherate (1.15 mL, 9.28 mmol) and the mixture was allowed to warm up to rt over 30 min. It was then re-cooled to 0° C. and to it was added a solution of benzyl 4-(2-methoxy-4-nitrophenyl)-5,6-dihydropyridine-1(2H)-carboxylate (2.85 g, 7.74 mmol) in THF (9.5 mL). The resulting mixture was allowed to warm to rt over 1 h. The reaction mixture was cooled again to 0° C., and EtOH (9.50 mL), 6M NaOH (16.0 mL, 95.9 mmol) and hydrogen peroxide (2.47 mL, 24.2 mmol) were sequentially added. The final mixture was warmed to rt for 1 h. The reaction was quenched with water and the mixture was extracted with ethyl acetate (50 mL×3). The combined organic layers were dried over Na₂SO₄ and concentrated. The crude was purified by column chromatography (Hexane/EtOAc=10-50%) to give benzyl 3-hydroxy-4-(2-methoxy-4-nitrophenyl)piperidine-1-carboxylate (800 mg, 27%) as a pale yellow solid. MS (m/z): [M+H]⁺: 387.

### Step 2.2

To a stirring mixture of dimethylsulfoxide (456 uL, 6.42 mmol) in DCM (7.0 mL) cooled to −78° C. was added oxalyl chloride (536 uL, 6.21 mmol) and the mixture was stirred for 15 min. To it was added a solution of benzyl 3-hydroxy-4-(2-methoxy-4-nitrophenyl)piperidine-1-carboxylate (800 mg, 2.07 mmol) in DCM (7.0 mL). The resulting mixture was stirred for 1.5 h. Triethylamine (1.75 mL, 12.4 mmol) was added and the reaction was warmed to rt. After cooling, the reaction was quenched with water and the mixture was extracted with ethyl acetate (3×50 mL). The combined organic layers were dried over Na₂SO₄ and concentrated in vacuo yielding benzyl 4-(2-methoxy-4-nitrophenyl)-3-oxopiperidine-1-carboxylate (796 mg, 100%) as an orange sticky oil. MS (m/z): [M+H]⁺: 384.

### Step 2.3

To a solution of benzyl 4-(2-methoxy-4-nitrophenyl)-3-oxopiperidine-1-carboxylate (650 mg, 1.69 mmol) in DCM (26.0 mL) cooled to 0° C. was added (diethylamino)sulfur trifluoride (628 uL, 5.07 mmol). The resulting mixture was allowed to warm to rt, and stirred for 16 hours. The reaction was quenched with sat. NaHCO₃ aq. and the mixture was extracted with ethyl acetate (3×50 mL). The combined organic layers were dried over Na₂SO₄ and concentrated in vacuo. The crude was purified by column chromatography (Hexane/EtOAc=0-30%) to give the title compound (300 mg, 44%) as a yellow oil. MS (m/z): [M+H]⁺: 407.

### Step 2.3: 4-(3,3-difluoropiperidin-4-yl)-3-methoxyaniline

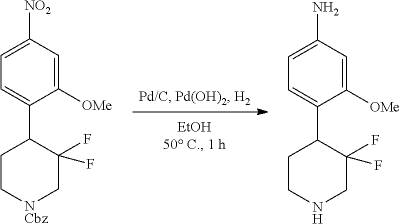 [SMILES: COc1cc([N+](=O)[O-])ccc1C1CCN(C(=O)OCc2ccccc2)CC1(F)F ; "Pd/C, Pd(OH)2, H2" ; COc1cc(N)ccc1C1CCNCC1(F)F]

The title compound was prepared following general procedure H, starting from benzyl 3,3-difluoro-4-(2-methoxy-4-nitrophenyl)piperidine-1-carboxylate with a mixture of Pd/C and Pd(OH)₂ (1:1) in EtOH. MS (m/z): [M+H]⁺: 243.

### Example 123: Preparation of 5-methoxy-6-(4-(oxetan-3-yl)piperazin-1-yl)pyridin-3-amine (Intermediate

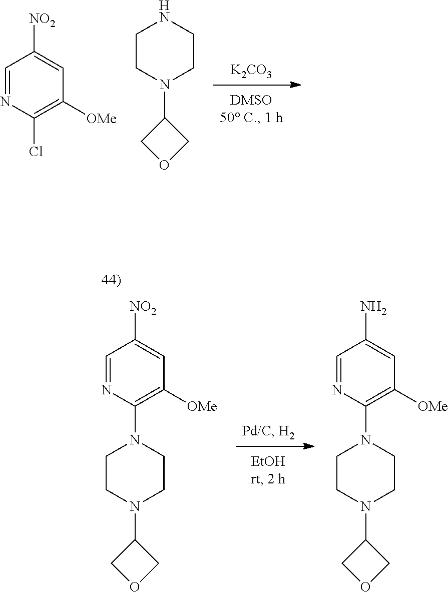

The title compound was prepared following general procedure G, starting from 2-chloro-3-methoxy-5-nitropyridine and 1-(oxetan-3-yl)piperazine in DMSO followed by nitro reduction employing the general nitro reduction procedure I. MS (m/z): [M+H]⁺: 265.

### Example 124: Preparation of Compound 51

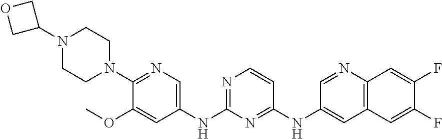 [SMILES: COc1cc(Nc2nccc(Nc3cnc4cc(F)c(F)cc4c3)n2)cnc1N1CCN(C2COC2)CC1]

Compound 51 was prepared from the corresponding intermediate according to general procedure F. MS (m/z): [M+H]⁺ 521. ¹H NMR (400 MHz, DMSO): δ 9.96 (s, 1H), 9.14 (s, 1H), 9.01 (s, 1H), 8.91 (s, 1H), 8.19 (d, J=2.1 Hz, 1H), 8.11 (d, J=5.7 Hz, 1H), 7.93 (dd, J=11.5, 8.0 Hz, 1H), 7.74 (t, J=10.2 Hz, 1H), 7.52 (s, 1H), 6.32 (d, J=5.7 Hz, 1H), 4.57 (t, J=6.5 Hz, 2H), 4.48 (t, J=6.1 Hz, 2H), 3.67 (s, 3H), 3.51-3.43 (m, 1H), 2.45-2.38 (m, 4H), 4H are overlapping with DMSO peak.

### Example 125: Preparation of 2-(4-(5-amino-3-methoxypyridin-2-yl)piperazin-2-yl)propan-2-ol (Intermediate 45)

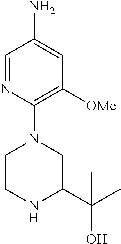 [SMILES: COc1cc(N)cnc1N1CCNC(C(C)(C)O)C1]

### Step 1: 1-tert-butyl 3-methyl 4-benzylpiperazine-1,3-dicarboxylate

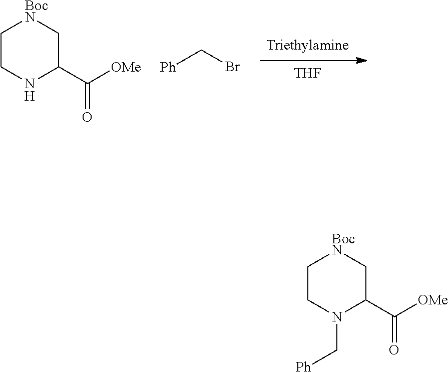 [SMILES: COC(=O)C1CN(C(=O)OC(C)(C)C)CCN1 ; BrCc1ccccc1 ; "Triethylamine" ; COC(=O)C1CN(C(=O)OC(C)(C)C)CCN1Cc1ccccc1 ; "THF"]

A solution of 1-tert-butyl 3-methyl piperazine-1,3-dicarboxylate (1.50 g, 5.83 mmol), triethylamine (2.46 mL, 17.5 mmol), and benzyl bromide (693 uL, 5.83 mmol) in THF (25 mL) was stirred at rt for 16 hours. Additional benzyl bromide (353 uL, 2.92 mmol) was added to the reaction, and the mixture was stirred for 5 hours at 50° C. The reaction mixture was filtered and the filtrate was concentrated. The crude was purified by column chromatography (Hexane/EtOAc=0-50%) to give the title compound (980 mg, 50%) as a colorless oil. MS (m/z): [M+H]⁺: 335.

### Step 2: 2-(1-benzylpiperazin-2-yl)propan-2-ol

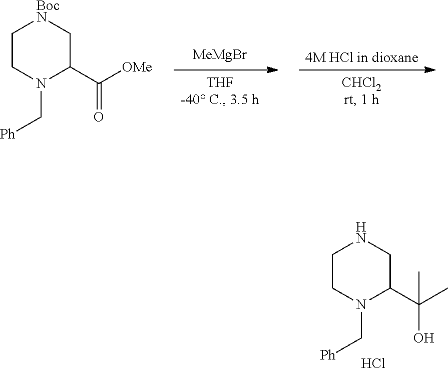

### Step A

To a solution of 1-tert-butyl 3-methyl 4-benzylpiperazine-1,3-dicarboxylate (500 mg, 1.50 mmol) in THF (10 mL) at −40° C. was added dropwise methylmagnesium bromide (1.74 mL, 5.23 mmol) (3M in THF). The reaction was stirred for 3.5 h at −40° C. The reaction was warmed to 0° C. and stirred for another 30 min. The reaction mixture was quenched by dropwise addition of saturated ammonium chloride solution at 0° C. The reaction mixture was extracted with EtOAc three times. The combined organic phase was dried over Na₂SO₄, filtered, and concentrated under reduced pressure. The crude was purified by column chromatography (Hexane/EtOAc=0-50%) to give tert-butyl 4-benzyl-3-(2-hydroxypropan-2-yl)piperazine-1-carboxylate (287 mg, 57%) as a colorless oil. MS (m/z): [M+H]⁺: 335.

### Step B

tert-butyl 4-benzyl-3-(2-hydroxypropan-2-yl)piperazine-1-carboxylate (287 mg, 858 umol) was dissolved in 4M HCl in Dioxane (3 mL) and CH₂Cl₂ (1.5 mL). The solution was stirred for 1 h. All volatile was removed under reduced pressure. The crude was dissolved in EtOAc and basified with sat. NaHCO₃ aq., then extracted with EtOAc three times. The combined organic phase was dried over Na₂SO₄, filtered, and concentrated under reduced pressure to give the title compound (232 mg, 100%) as a white solid. MS (m/z): [M+H]⁺: 235.

### Step 3: 2-(4-(5-amino-3-methoxypyridin-2-yl)piperazin-2-yl)propan-2-ol

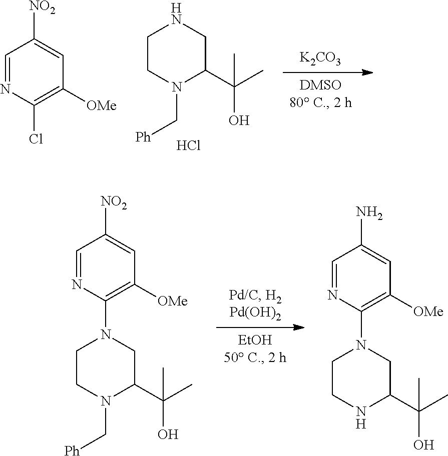

The title compound was prepared following general procedure G, starting from 2-chloro-3-methoxy-5-nitropyridine and 2-(1-benzylpiperazin-2-yl)propan-2-ol in DMSO followed by nitro reduction employing the general nitro reduction procedure I with a mixture of Pd/C and Pd(OH)₂ (1:1). MS (m/z): [M+H]⁺: 267.

### Example 126: Preparation of Compound 52

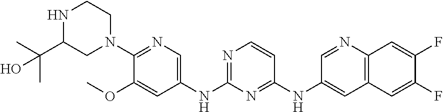 [SMILES: COc1cc(Nc2nccc(Nc3cnc4cc(F)c(F)cc4c3)n2)cnc1N1CCNC(C(C)(C)O)C1]

Compound 52 was prepared from the corresponding intermediate according to general procedure C. MS (m/z): [M+H]⁺ 523.5. H NMR (500 MHz, DMSO) δ (ppm): 9.95 (s, 1H), 9.12 (s, 1H), 9.01 (s, 1H), 8.92 (s, 1H), 8.17 (d, J=2.1 Hz, 1H), 8.11 (d, J=5.7 Hz, 1H), 7.99-7.89 (m, 1H), 7.74 (s, 1H), 7.51 (s, 1H), 6.32 (d, J=5.7 Hz, 1H), 4.32 (s, 1H), 3.76 (d, J=11.4 Hz, 1H), 3.72-3.64 (m, 4H), 3.00 (d, J=11.3 Hz, 1H), 2.88-2.81 (m, 1H), 2.63-2.58 (m, 2H), 2.40 (t, J=11.0 Hz, 1H), 2.09 (s, 1H), 1.13 (s, 3H), 1.11 (s, 3H).

### Example 127: Preparation of 7,8-dihydro-5H-pyrano[4,3-b]pyridin-3-amine (Intermediate 47)

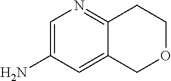 [SMILES: Nc1cnc2c(c1)COCC2]

### Step 1: 3-nitro-7,8-dihydro-5H-pyrano[4,3-b]pyridine

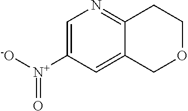

The title compound was prepared from tetrahydro-4H-pyran-4-one according to 3-nitropyridine synthesis general procedure R and the crude material was used directly in the next step. [M+H]⁺: 180.9.

### Step 2: 7,8-dihydro-5H-pyrano[4,3-b]pyridin-3-amine

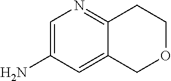 [SMILES: Nc1cnc2c(c1)COCC2]

The title compound was prepared from 3-nitro-7,8-dihydro-5H-pyrano[4,3-b]pyridine according to nitro reduction general procedure H and the crude material was used directly in the next step. [M+H]⁺: 151.1.

### Example 128: Preparation of Compound 53

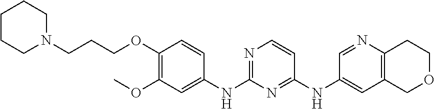 [SMILES: COc1cc(Nc2nccc(Nc3cnc4c(c3)COCC4)n2)ccc1OCCCN1CCCCC1]

Compound 53 was prepared from the corresponding intermediate according to general procedure C. MS (m/z): [M+H]⁺ 491.5. H NMR (500 MHz, DMSO) δ 9.43 (s, 1H), 8.96 (s, 1H), 8.52 (s, 1H), 8.01 (d, J=5.7 Hz, 1H), 7.95 (d, J=2.0 Hz, 1H), 7.27 (s, 1H), 7.15 (d, J=8.7 Hz, 1H), 6.86 (d, J=8.8 Hz, 1H), 6.18 (d, J=5.8 Hz, 1H), 4.62 (s, 2H), 4.01-3.90 (m, 5H), 3.71-3.62 (m, 4H), 2.82 (t, J=5.8 Hz, 2H), 2.42 (broad m, 4H), 1.88-1.80 (broad m, 2H), 1.52 (broad m, 4H), 1.40 (broad m, 2H).

### Example 129: Preparation of 6,8-dihydro-5H-pyrano[3,4-b]pyridin-3-amine (Intermediate 47)

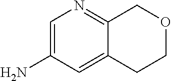 [SMILES: Nc1cnc2c(c1)CCOC2]

### Step 1: 3-nitro-6,8-dihydro-5H-pyrano[3,4-b]pyridine

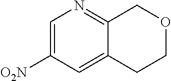 [SMILES: O=[N+]([O-])c1cnc2c(c1)CCOC2]

The title compound was prepared from tetrahydro-4H-pyran-3-one according to 3-nitropyridine synthesis general procedure R and the crude material was used directly in the next step. [M+H]⁺: 180.9.

### Step 2: 6,8-dihydro-5H-pyrano[3,4-b]pyridin-3-amine

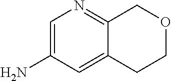 [SMILES: Nc1cnc2c(c1)CCOC2]

The title compound was prepared from crude 3-nitro-6,8-dihydro-5H-pyrano[3,4-b]pyridine according to nitro reduction general procedure H and was purified by flash chromatography. ¹H NMR (500 MHz, CDCl3) δ 7.88 (d, J=2.7 Hz, 1H), 6.73 (d, J=2.5 Hz, 1H), 4.68 (s, 2H), 3.91 (t, J=5.8 Hz, 2H), 3.5 (broad s, 2H), 2.76 (t, J=5.7 Hz, 2H).

### Example 130: Preparation of Compound 54

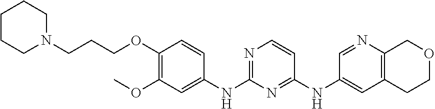 [SMILES: COc1cc(Nc2nccc(Nc3cnc4c(c3)CCOC4)n2)ccc1OCCCN1CCCCC1]

Compound 54 was prepared from the corresponding intermediate according to general procedure C. MS (m/z): [M+H]⁺ 489.5 (M−1). H NMR (500 MHz, DMSO) δ 9.43 (s, 1H), 8.98 (s, 1H), 8.53 (s, 1H), 8.02 (m, 2H), 7.26 (s, 1H), 7.16 (d, J=8.0 Hz, 1H), 6.85 (d, J=8.7 Hz, 1H), 6.18 (d, J=5.7 Hz, 1H), 4.61 (s, 2H), 3.93 (t, J=6.5 Hz, 2H), 3.89 (t, J=5.7 Hz, 2H), 3.64 (s, 3H), 2.76 (t, J=5.5 Hz, 2H), 2.39-2.31 (m, 6H), 1.85-1.80 (m, 2H), 1.53-1.46 (m, 4H), 1.41-1.35 (m, 2H).

### Example 131: Preparation of 7,8-dihydro-5H-thiopyrano[4,3-b]pyridin-3-amine (Intermediate 48)

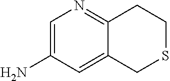 [SMILES: Nc1cnc2c(c1)CSCC2]

### Step 1: 3-nitro-7,8-dihydro-5H-thiopyrano[4,3-b]pyridine

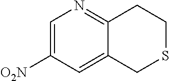 [SMILES: O=[N+]([O-])c1cnc2c(c1)CSCC2]

The title compound was prepared from tetrahydro-4H-thiopyran-4-one according to 3-nitropyridine synthesis general procedure R and the crude material was used directly in the next step. [M+H]⁺: 197.1.

### Step 2: 7,8-dihydro-5H-thiopyrano[4,3-b]pyridin-3-amine

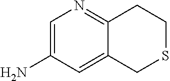 [SMILES: Nc1cnc2c(c1)CSCC2]

The title compound was prepared from crude 3-nitro-7,8-dihydro-5H-thiopyrano[4,3-b]pyridine according to nitro reduction general procedure I and was purified by flash chromatography. [M+H]⁺: 167.1.

### Example 132: Preparation of Compound 55

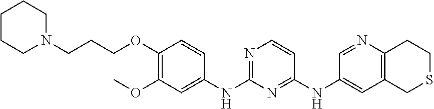 [SMILES: COc1cc(Nc2nccc(Nc3cnc4c(c3)CSCC4)n2)ccc1OCCCN1CCCCC1]

Compound 55 was prepared from the corresponding intermediate according to general procedure C. MS (m/z): [M+H]⁺ 507.5. H NMR (500 MHz, DMSO) δ 9.46 (broad m, 1H), 8.95 (broad s, 1H), 8.55 (d, J=0.5 Hz, 1H), 8.02 (m, 2H), 7.29 (s, 1H), 7.22-7.11 (m, 1H), 6.92 (d, J=8.7 Hz, 1H), 6.24 (d, J=5.8 Hz, 1H), 4.01 (t, J=5.8 Hz, 2H), 3.72 (s, 2H), 3.67 (s, 3H), 3.51 (d, J=11.8 Hz, 2H), 3.26-3.19 (m, 2H), 3.08-2.87 (m, 6H), 2.16-2.07 (m, 2H), 1.85 (d, J=14.3 Hz, 2H), 1.77-1.58 (m, 3H), 1.40 (q, J=13.3 Hz, 1H).

### Example 133: Preparation of (5R,8S)-5,6,7,8-tetrahydro-5,8-methanoquinolin-3-amine (Intermediate 49)

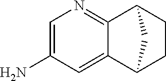 [SMILES: Nc1cnc2c(c1)[C@@H]1CC[C@H]2C1]

### Step 1: (5R,8S)-3-nitro-5,6,7,8-tetrahydro-5,8-methanoquinoline

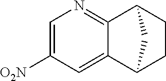 [SMILES: O=[N+]([O-])c1cnc2c(c1)[C@@H]1CC[C@H]2C1]

The title compound was prepared from norcamphor according to 3-nitropyridine synthesis general procedure R and was purified by chromatography. [M+H]⁺: 190.6

### Step 2: (5R,8S)-5,6,7,8-tetrahydro-5,8-methanoquinolin-3-amine

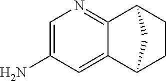 [SMILES: Nc1cnc2c(c1)[C@@H]1CC[C@H]2C1]

The title compound was prepared from (5R,8S)-3-nitro-5,6,7,8-tetrahydro-5,8-methanoquinoline according to nitro reduction general procedure H and the crude material was used directly in the next step. [M+H]⁺: 161.2.

### Example 134: Preparation of Compound 56

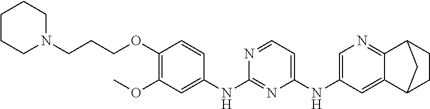 [SMILES: COc1cc(Nc2nccc(Nc3cnc4c(c3)C3CCC4C3)n2)ccc1OCCCN1CCCCC1]

Compound 56 was prepared from the corresponding intermediate according to general procedure C. MS (m/z): [M+H]⁺ 501.6. H NMR (500 MHz, DMSO) δ 9.28 (s, 1H), 8.93 (s, 1H), 8.25 (s, 1H), 8.03 (s, 1H), 7.97 (d, J=5.7 Hz, 1H), 7.28 (d, J=0.5 Hz, 1H), 7.18 (d, J=8.7 Hz, 1H), 6.83 (d, J=8.7 Hz, 1H), 6.14 (d, J=5.7 Hz, 1H), 3.94 (t, J=6.1 Hz, 2H), 3.65 (s, 3H), 3.25 (s, 1H), 2.48 (broad m, overlap with DMSO, 8H) 1.98-1.83 (m, 4H), 1.73 (d, J=8.6 Hz, 1H), 1.59-1.50 (m, 4H), 1.41 (s, 2H), 1.18-1.07 (m, 2H).

### Example 135: Preparation of 6,7,8,9-tetrahydro-5H-cyclohepta[b]pyridin-3-amine (Intermediate 50)

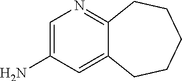 [SMILES: Nc1cnc2c(c1)CCCCC2]

### Step 1: 3-nitro-6,7,8,9-tetrahydro-5H-cyclohepta[b]pyridine

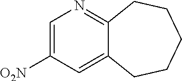 [SMILES: O=[N+]([O-])c1cnc2c(c1)CCCCC2]

The title compound was prepared in 94% yield from cycloheptanone according to 3-nitropyridine synthesis general procedure R and was purified by chromatography. [M+H]⁺: 193.4.

### Step 2: 6,7,8,9-tetrahydro-5H-cyclohepta[b]pyridin-3-amine

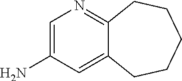 [SMILES: Nc1cnc2c(c1)CCCCC2]

The title compound was prepared in 65% yield from 3-nitro-6,7,8,9-tetrahydro-5H-cyclohepta[b]pyridine according to nitro reduction general procedure H and was purified by chromatography. [M+H]⁺: 163.2.

### Example 136: Preparation of Compound 57

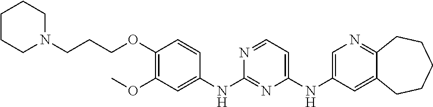 [SMILES: COc1cc(Nc2nccc(Nc3cnc4c(c3)CCCCC4)n2)ccc1OCCCN1CCCCC1]

Compound 57 was prepared from the corresponding intermediate according to general procedure C. MS (m/z): [M+H]⁺ 503.5. H NMR (500 MHz, DMSO) δ 9.33 (s, 1H), 8.97 (s, 1H), 8.43 (s, 1H), 8.00 (d, J=5.7 Hz, 1H), 7.97 (d, J=2.4 Hz, 1H), 7.28 (s, 1H), 7.20 (dd, J=8.4, 1.7 Hz, 1H), 6.84 (d, J=8.8 Hz, 1H), 6.17 (d, J=5.7 Hz, 1H), 3.94 (t, J=6.4 Hz, 2H), 3.66 (s, 3H), 2.96-2.83 (m, 2H), 2.74-2.65 (m, 2H), 2.44 (overlap with solvent, broad s, 6H), 1.93-1.76 (m, 4H), 1.62-1.49 (m, 8H), 1.41 (s, 2H).

### Example 137: Preparation of 3,4-dihydro-2H-pyrano[3,2-b]pyridin-7-amine (Intermediate 51)

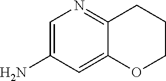 [SMILES: Nc1cnc2c(c1)OCCC2]

### Step 1: 4-(3,4-dihydro-2H-pyran-5-yl)morpholine

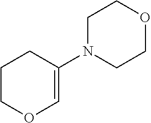 [SMILES: C1=C(N2CCOCC2)CCCO1]

To a solution of morpholine (1.31 mL, 14.8 mmol) in Toluene (20.0 mL) was added dihydro-2H-pyran-3(4H)-one (1.00 g, 9.99 mmol). The mixture was refluxed for 4 hours with water removal. The mixture was concentrated in vacuo to give the title compound which was used without further purification. ¹H NMR (400 MHz, CDCl₃) δ (ppm): 6.04 (t, J=1.2 Hz, 1H), 3.88-3.80 (m, 2H), 3.72 (ddd, J=9.9, 6.1, 4.0 Hz, 4H), 2.71-2.65 (m, 4H), 2.16-2.08 (m, 2H), 1.93-1.84 (m, 2H).

### Step 2: 7-nitro-3,4-dihydro-2H-pyrano[3,2-b]pyridine

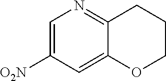 [SMILES: O=[N+]([O-])c1cnc2c(c1)OCCC2]

The title compound was prepared from crude 4-(3,4-dihydro-2H-pyran-5-yl)morpholine according to 3-nitropyridine synthesis general procedure R and was purified by flash chromatography. ¹H NMR (400 MHz, CDCl₃) δ (ppm): 8.95 (s, 1H), 7.86 (d, J=2.1 Hz, 1H), 4.38-4.16 (m, 2H), 3.07 (t, J=6.4 Hz, 2H), 2.24-2.05 (m, 2H).

### Step 3: 3,4-dihydro-2H-pyrano[3,2-b]pyridin-7-amine

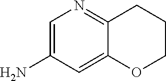 [SMILES: Nc1cnc2c(c1)OCCC2]

The title compound was prepared from 7-nitro-3,4-dihydro-2H-pyrano[3,2-b]pyridine according to nitro reduction general procedure H and the crude material was used directly in the next step. [M+H]⁺: 151.

### Example 138: Preparation of Compound 58

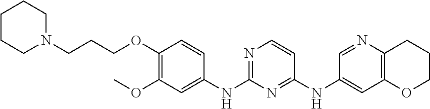 [SMILES: COc1cc(Nc2nccc(Nc3cnc4c(c3)OCCC4)n2)ccc1OCCCN1CCCCC1]

Compound 58 was prepared from the corresponding intermediate according to general procedure C. MS (m/z): [M+H]⁺ 491.5. H NMR (500 MHz, DMSO) δ 9.43 (s, 1H), 9.07 (s, 1H), 8.33 (d, J=1.9 Hz, 1H), 8.07 (d, J=5.7 Hz, 1H), 7.79 (d, J=1.9 Hz, 1H), 7.38 (s, 1H), 7.30 (dd, J=8.6, 2.0 Hz, 1H), 6.90 (d, J=8.8 Hz, 1H), 6.23 (d, J=5.8 Hz, 1H), 4.24-4.16 (m, 2H), 4.00 (t, J=6.4 Hz, 2H), 3.74 (s, 3H), 2.86 (t, J=6.5 Hz, 2H), 2.42 (broad s, 6H), 2.14-2.02 (m, 2H), 1.91 (broad s, 2H), 1.57 (broad s, 4H), 1.46 (broad s, 2H).

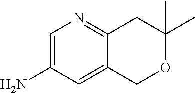

### Step 1: 7,7-dimethyl-3-nitro-7,8-dihydro-5H-pyrano[4,3-b]pyridine

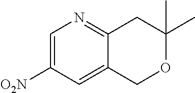

The title compound was prepared from 2,2-dimethyldihydro-4H-pyran-4-one according to 3-nitropyridine synthesis general procedure R and was purified by flash chromatography. ¹H NMR (500 MHz, CDCl3) δ (ppm): 9.21 (d, J=2.4 Hz, 1H), 8.12 (d, J=2.5 Hz, 1H), 4.85 (s, 2H), 2.94 (s, 2H), 1.30 (s, 6H).

### Step 2: 7,7-dimethyl-7,8-dihydro-5H-pyrano[4,3-b]pyridin-3-amine

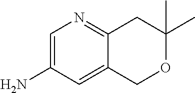

The title compound was prepared from 7,7-dimethyl-3-nitro-7,8-dihydro-5h-pyrano[4,3-b]pyridine according to nitro reduction general procedure H and the crude material was used directly in the next step. [M+H]⁺: 179.2.

### Example 140: Preparation of Compound 59

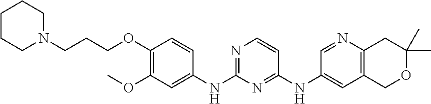 [SMILES: COc1cc(Nc2nccc(Nc3cnc4c(c3)COC(C)(C)C4)n2)ccc1OCCCN1CCCCC1]

Compound 59 was prepared from the corresponding intermediate according to general procedure C. MS (m/z): [M+H]⁺ 519.6. H NMR (500 MHz, DMSO) δ 9.42 (s, 1H), 8.95 (s, 1H), 8.51 (d, J=0.8 Hz, 1H), 8.00 (d, J=5.7 Hz, 1H), 7.98 (d, J=2.2 Hz, 1H), 7.25 (s, 1H), 7.14 (d, J=8.2 Hz, 1H), 6.84 (d, J=8.7 Hz, 1H), 6.17 (d, J=5.8 Hz, 1H), 4.62 (s, 2H), 3.93 (t, J=6.5 Hz, 2H), 3.64 (s, 3H), 2.70 (s, 2H), 2.43-2.23 (m, 6H), 1.87-1.78 (m, 2H), 1.49 (p, J=5.4 Hz, 4H), 1.43-1.30 (m, 2H), 1.23 (s, 6H).

### Example 141: Preparation of 6,7,8,9-tetrahydro-5H-5,8-epoxycylohepta[b]pyridin-3-amine (Intermediate

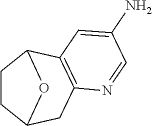 [SMILES: Nc1cnc2c(c1)C1CCC(C2)O1]

### Step 1: 3-nitro-6,7,8,9-tetrahydro-5H-5,8-epoxycyclohepta[b]pyridine

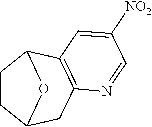 [SMILES: O=[N+]([O-])c1cnc2c(c1)C1CCC(C2)O1]

The title compound was prepared from 8-oxabicyclo[3.2.1]octan-3-one according to 3-nitropyridine synthesis general procedure R and was purified by flash chromatography. ¹H NMR (500 MHz, CDCl3) δ (ppm): 9.25 (d, J=2.5 Hz, 1H), 8.11 (d, J=2.5 Hz, 1H), 5.18 (d, J=6.5 Hz, 1H), 4.96-4.83 (m, 1H), 3.49 (dd, J=18.4, 5.7 Hz, 1H), 2.82 (d, J=18.3 Hz, 1H), 2.38-2.25 (m, 2H), 2.08-1.95 (m, 1H), 1.83-1.69 (m, 1H).

### Step 2: 6,7,8,9-tetrahydro-5H-5,8-epoxycyclohepta[b]pyridin-3-amine

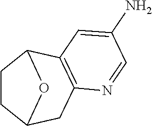 [SMILES: Nc1cnc2c(c1)C1CCC(C2)O1]

The title compound was prepared from 3-nitro-6,7,8,9-tetrahydro-5h-5,8-epoxycyclohepta[b]pyridine according to nitro reduction general procedure H and the crude material was used directly in the next step. [M+H]⁺: 177.1.

### Example 142: Preparation of Compound 60

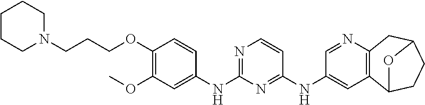 [SMILES: COc1cc(Nc2nccc(Nc3cnc4c(c3)C3CCC(C4)O3)n2)ccc1OCCCN1CCCCC1]

Compound 60 was prepared from the corresponding intermediate according to general procedure C. MS (m/z): [M+H]⁺ 517.5. H NMR (500 MHz, DMSO) δ 9.39 (s, 1H), 8.93 (s, 1H), 8.49 (s, 1H), 8.00 (d, J=5.8 Hz, 1H), 7.94 (d, J=2.3 Hz, 1H), 7.25 (s, 1H), 7.16 (d, J=9.5 Hz, 1H), 6.84 (d, J=8.7 Hz, 1H), 6.17 (d, J=5.7 Hz, 1H), 4.95 (d, J=5.2 Hz, 1H), 4.74 (dd, J=7.3, 5.5 Hz, 1H), 3.92 (t, J=6.5 Hz, 2H), 3.65 (s, 3H), 3.15 (dd, J=17.0, 5.2 Hz, 1H), 2.54 (d, J=17.0 Hz, 1H), 2.38 (m, 6H), 2.15-2.01 (m, 2H), 1.87-1.79 (m, 3H), 1.69-1.63 (m, 1H), 1.53-1.46 (m, 4H), 1.41-1.34 (m, 2H).

### Example 143: Preparation of 2-methyl-3,4-dihydro-2H-thiopyrano[3,2-b]pyridin-7-amine (Intermediate

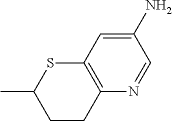

### Step 1: methyl 4-((2-methoxy-2-oxoethyl)thio)pentanoate

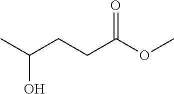

Potassium hydroxide (2.23 g, 33.8 mmol) was dissolved in 15 mL of water and then γ-Valerolactone (4.77 mL, 47.5 mmol) was added as a solution in 20 mL of water. The reaction was heated to reflux for 3 hours then was concentrated and chased with iPrOH and toluene several times to give a hard white solid. 50 mL of DMF was added followed by iodomethane (14.9 mL, 238 mmol) and the mixture was stirred at room temperature for 24 hours. The reaction was diluted with water and extracted three times EtOAc, then the combined organic layers were washed three times with 5% LiCl then dried over magnesium sulfate, filtered and concentrated to give the title compound as a yellow oil in 46% yield. ¹H NMR (500 MHz, CDCl3) δ (ppm): 3.82-3.71 (m, 1H), 3.61 (s, 3H), 2.39 (t, J=7.4 Hz, 2H), 1.82-1.60 (m, 3H), 1.15 (d, J=7.6 Hz, 3H).

### Step 2: methyl 6-methyl-3-oxotetrahydro-2H-thiopyran-2-carboxylate

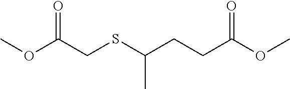

A solution of methyl 4-hydroxypentanoate (2.80 g, 21.2 mmol) and triethylamine (4.43 mL, 31.8 mmol) in THF (80.0 mL) was cooled to 0° C. and methanesulfonyl chloride (2.14 mL, 27.5 mmol) was added dropwise then the mixture was warmed to room temperature and stirred for 90 minutes. The mixture was then diluted with EtOAc, washed with NH4Cl then concentrated to give 3.4 g of crude mesylate.

The residue was dissolved in DMF (42.4 mL) then methyl thioglycolate (2.25 g, 21.2 mmol) and K₂CO₃ (5.85 g, 42.4 mmol) were added and the mixture was heated to 60° C. after 1 hour. Additional DMF (42.4 mL) was added and stirring continued for two more hours. The reaction was cooled, diluted with 1% Na₂CO₃, extracted three times with Et₂O then the combined organic layers were washed three times with water, dried over magnesium sulfate, filtered and concentrated. The residue was purified by flash chromatography using 0 to 25% EtOAc in hexanes to give the title compound (3.69 g, 79%) as a colorless oil. ¹H NMR (500 MHz, CDCl3) δ (ppm): 3.72 (s, 3H), 3.67 (s, 3H), 3.25 (s, 2H), 2.94 (m, 1H), 2.46 (m, 2H), 1.88 (d, J=7.3 Hz, 1H), 1.85 (dd, J=7.3, 0.9 Hz, 1H), 1.30 (d, J=6.8 Hz, 3H).

### Step 3: 6-methyldihydro-2H-thiopyran-3(4H)-one

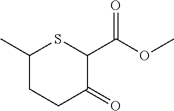

A solution of methyl 4-((2-methoxy-2-oxoethyl)thio)pentanoate (3.69 g, 16.8 mmol) in THF (70 mL) was cooled in an ice bath then tBuOK (3.84 g, 33.5 mmol) was added and the solution was stirred at room temperature for 2 hours. The reaction diluted with 1M HCl and extracted 3 times with DCM then dried over magnesium sulfate, filtered and concentrated to give the title compound (3.00 g, 95%) as an orange oil which was used in the next step without further purification. ¹H NMR (500 MHz, CDCl3) δ (ppm): 12.15 (s, 1H), 3.81 (s, 3H), 3.26-3.05 (m, 1H), 2.50-2.40 (m, 2H), 2.18-2.08 (m, 1H), 1.87-1.75 (m, 1H), 1.32 (d, J=6.8 Hz, 3H).

### Step 4: 6-methyldihydro-2H-thiopyran-3(4H)-one

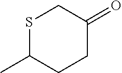

Aqueous sulfuric acid (10%, 70 mL), was added to crude methyl 4-((2-methoxy-2-oxoethyl)thio)pentanoate (3 g, 19 mmol) and the mixture was heated to reflux for 3 hours. The reaction was then cooled, extracted three times with EtOAc, dried over magnesium sulfate, filtered and concentrated. The residue was purified by Flash chromatography (SiO₂, EtOAc/Hexane) to give the title compound as an orange oil (1.39 g, 56%). ¹H NMR (400 MHz, cdcl3) δ (ppm): 3.43 (dd, J=13.1, 0.6 Hz, 1H), 3.31-3.18 (m, 1H), 3.00 (dd, J=13.2, 1.7 Hz, 1H), 2.53-2.36 (m, 3H), 2.22-2.05 (m, 1H), 1.31 (d, J=6.8 Hz, 3H).

### Step 5: 2-methyl-7-nitro-3,4-dihydro-2H-thiopyrano[3,2-b]pyridine

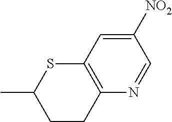

The title compound was prepared from 6-methyldihydro-2H-thiopyran-3(4H)-one according to 3-nitropyridine synthesis general procedure R and was purified by flash chromatography. ¹H NMR (500 MHz, CDCl3) δ (ppm): 8.96 (d, J=2.5 Hz, 1H), 8.13 (d, J=2.4 Hz, 1H), 3.53-3.44 (m, 1H), 3.19 (ddd, J=18.1, 5.3, 4.3 Hz, 1H), 3.05 (ddd, J=18.1, 10.9, 4.7 Hz, 1H), 2.35 (dtd, J=13.6, 5.0, 3.2 Hz, 1H), 2.00-1.86 (m, 1H), 1.42 (d, J=6.8 Hz, 3H).

### Step 6: 2-methyl-3,4-dihydro-2H-thiopyrano[3,2-b]pyridin-7-amine

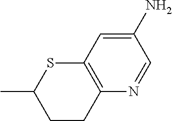

The title compound was prepared from 2-methyl-7-nitro-3,4-dihydro-2H-thiopyrano[3,2-b]pyridine according to nitro reduction general procedure H and the crude material was used directly in the next step. [M+H]⁺: 181.2.

### Example 144: Preparation of Compound 61

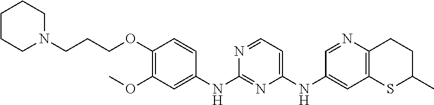 [SMILES: COc1cc(Nc2nccc(Nc3cnc4c(c3)SC(C)CC4)n2)ccc1OCCCN1CCCCC1]

Compound 61 was prepared from the corresponding intermediate according to general procedure C. MS (m/z): [M+H]⁺ 521.6. H NMR (500 MHz, DMSO) δ 9.37 (s, 1H), 9.00 (s, 1H), 8.37 (d, J=0.9 Hz, 1H), 8.00 (d, J=5.7 Hz, 1H), 7.94 (d, J=2.4 Hz, 1H), 7.27-7.16 (m, 2H), 6.88 (d, J=8.7 Hz, 1H), 6.15 (d, J=5.7 Hz, 1H), 3.93 (t, J=6.5 Hz, 2H), 3.67 (s, 3H), 3.44 (m, 1H), 2.92-2.84 (m, 2H), 2.43-2.20 (m, 7H), 1.86-1.73 (m, 3H), 1.55-1.44 (m, 4H), 1.40 (m, 2H), 1.33 (d, J=6.8 Hz, 3H).

### Example 145: Preparation of 6-methyl-5,6,7,8-tetrahydroquinolin-3-amine (Intermediate 55)

### Step 1: 6-methyl-3-nitro-5,6,7,8-tetrahydroquinoline

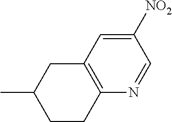

The title compound was prepared from 4-methylcyclohexanone according to 3-nitropyridine synthesis general procedure R and was purified by flash chromatography. [M+H]⁺: 193.2.

### Step 2: 6-methyl-5,6,7,8-tetrahydroquinolin-3-amine

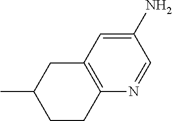

The title compound was prepared from 6-methyl-3-nitro-5,6,7,8-tetrahydroquinoline according to nitro reduction nitro reduction general procedure I and the crude material was used directly in the next step. [M+H]⁺: 163.2.

### Example 146: Preparation of Compound 62

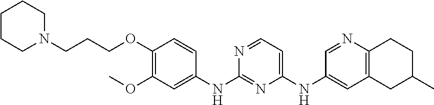 [SMILES: COc1cc(Nc2nccc(Nc3cnc4c(c3)CC(C)CC4)n2)ccc1OCCCN1CCCCC1]

Compound 62 was prepared from the corresponding intermediate according to general procedure C. MS (m/z): [M+H]⁺ 503.5. H NMR (400 MHz, dmso) δ 9.28 (s, 1H), 8.90 (s, 1H), 8.37 (s, 1H), 7.94 (d, J=5.7 Hz, 1H), 7.87 (d, J=2.3 Hz, 1H), 7.19 (s, 1H), 7.12 (dd, J=8.6, 1.8 Hz, 1H), 6.78 (d, J=8.8 Hz, 1H), 6.10 (d, J=5.7 Hz, 1H), 3.88 (t, J=6.4 Hz, 2H), 3.57 (s, 3H), 2.74 (dd, J=11.0, 7.3 Hz, 2H), 2.68-2.60 (m, 1H), 2.42-2.22 (m, 7H), 1.80 (dt, J=13.9, 9.0 Hz, 4H), 1.48-1.40 (m, 5H), 1.38-1.30 (m, 2H), 0.99 (d, J=6.4 Hz, 3H).

### Example 147: Preparation of 6,6-dimethyl-5,6,7,8-tetrahydroquinolin-3-amine (Intermediate 56)

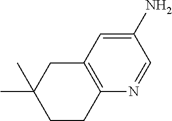

### Step 1: 6,6-dimethyl-3-nitro-5,6,7,8-tetrahydroquinoline

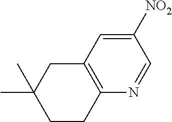

The title compound was prepared from 4,4-dimethyl-cyclohexanone according to 3-nitropyridine synthesis general procedure R and was purified by flash chromatography.

### Step 2: 6,6-dimethyl-5,6,7,8-tetrahydroquinolin-3-amine

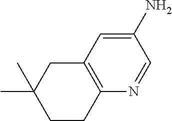

The title compound was prepared from 6,6-dimethyl-3-nitro-5,6,7,8-tetrahydroquinoline according to nitro reduction general procedure I and the crude material was used directly in the next step. [M+H]⁺: 177.2.

### Example 148: Preparation of Compound 63

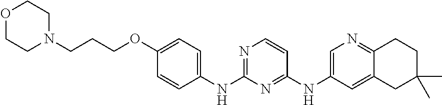 [SMILES: CC1(C)CCc2ncc(Nc3ccnc(Nc4ccc(OCCCN5CCOCC5)cc4)n3)cc2C1]

Compound 63 was prepared from the corresponding intermediate according to general procedure C. MS (m/z): [M+H]⁺ 489.5. H NMR (500 MHz, DMSO) δ 9.32 (s, 1H), 8.95 (s, 1H), 8.39 (s, 1H), 7.97 (d, J=5.7 Hz, 2H), 7.50 (d, J=8.8 Hz, 2H), 6.83 (d, J=9.1 Hz, 2H), 6.15 (d, J=5.7 Hz, 1H), 3.97 (t, J=6.5 Hz, 2H), 3.57 (t, J=4.5 Hz, 4H), 2.77 (t, J=6.7 Hz, 2H), 2.47 (s, 2H), 2.42 (t, J=7.3 Hz, 2H), 2.36 (s, 4H), 1.90-1.81 (m, 2H), 1.61 (t, J=6.9 Hz, 2H), 0.97 (s, 6H).

### Example 149: Preparation of 7,7-dimethyl-5,6,7,8-tetrahydroquinolin-3-amine (Intermediate 57)

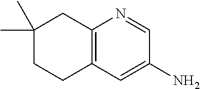

### Step 1: 7,7-dimethyl-3-nitro-5,6,7,8-tetrahydroquinoline

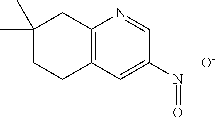

The title compound was prepared from 3,3-dimethylcyclohexanone according to 3-nitropyridine synthesis general procedure R and was purified by flash chromatography. ¹H NMR (400 MHz, cdcl3) δ (ppm): 9.18 (d, J=2.5 Hz, 1H), 8.19 (d, J=2.5 Hz, 1H), 3.01-2.86 (m, 2H), 2.80 (s, 2H), 1.66 (t, J=6.7 Hz, 2H), 1.05 (s, 6H).

### Step 2: 7,7-dimethyl-5,6,7,8-tetrahydroquinolin-3-amine

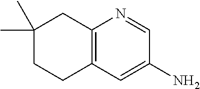

The title compound was prepared from 7,7-dimethyl-3-nitro-5,6,7,8-tetrahydroquinoline according to nitro reduction general procedure I and was purified by flash chromatography. [M+H]⁺: 177.1.

### Example 150: Preparation of Compound 64

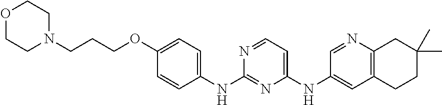 [SMILES: CC1(C)CCc2cc(Nc3ccnc(Nc4ccc(OCCCN5CCOCC5)cc4)n3)cnc2C1]

Compound 64 was prepared from the corresponding intermediate according to general procedure D. MS (m/z): [M+H]⁺ 489.5. ¹H NMR (500 MHz, DMSO) δ 9.33 (s, 1H), 8.97 (s, 1H), 8.41 (s, 1H), 7.97 (d, J=5.7 Hz, 1H), 7.94 (d, J=1.9 Hz, 1H), 7.50 (d, J=8.5 Hz, 2H), 6.82 (d, J=9.1 Hz, 2H), 6.14 (d, J=5.7 Hz, 1H), 3.95 (t, J=6.5 Hz, 2H), 3.57 (t, J=4.6 Hz, 4H), 2.70 (t, J=6.3 Hz, 2H), 2.54 (s, 2H), 2.41 (t, J=7.2 Hz, 2H), 2.36 (s, 4H), 1.89-1.82 (m, 2H), 1.53 (t, J=6.7 Hz, 2H), 0.98 (s, 6H).

### Example 151: Preparation of 3,3-dimethyl-2,3-dihydro-[1,4]dioxino[2,3-b]pyridin-7-amine (Intermediate

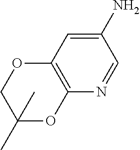

### Step 1: 7-bromo-3,3-dimethyl-2,3-dihydro-[1,4]dioxino[2,3-b]pyridine

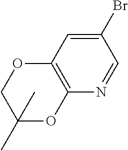

To a solution of 2-methylpropane-1,2-diol (368 mg, 4.00 mmol) in DMF (10.0 mL) was added sodium hydride 60% in dispersion in mineral oil (240 mg, 6.00 mmol). The mixture was stirred at room temperature for 20 minutes then 5-Bromo-2-chloro-3-fluoropyridine (421 mg, 2.00 mmol) was added. Stirring was continued at room temperature for 1 hour then heated to 100° C. for 5 hours. The mixture was diluted with EtOAc and washed once with 0.1M HCl then three times with 5% LiCl. The organic layer was dried over magnesium sulfate, filtered and concentrated. The residue was purified by flash chromatography (SiO₂, 40% EtOAc in hexanes). Fractions containing the title compound were combined and washed four times with 0.5M NaOH. The organic layer was dried over magnesium sulfate, filtered and concentrated to give the title compound (105 mg, 22%) as colorless oil. [M+H]⁺: 244.1.

### Step 2: 3,3-dimethyl-2,3-dihydro-[1,4]dioxino[2,3-b]pyridin-7-amine

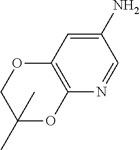

The title compound was prepared from 7-bromo-3,3-dimethyl-2,3-dihydro-[1,4]dioxino[2,3-b]pyridine following general procedure X and was purified by flash chromatography. [M+H]⁺: 181.

### Example 152: Preparation of Compound 65

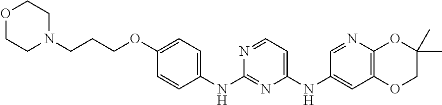 [SMILES: CC1(C)COc2cc(Nc3ccnc(Nc4ccc(OCCCN5CCOCC5)cc4)n3)cnc2O1]

Compound 65 was prepared from the corresponding intermediate according to general procedure D. MS (m/z): [M+H]⁺ 493.4. ¹H NMR (400 MHz, dmso) δ 9.27 (s, 1H), 8.99 (s, 1H), 7.95 (d, J=5.7 Hz, 1H), 7.89 (s, 1H), 7.80 (d, J=2.3 Hz, 1H), 7.54 (d, J=9.0 Hz, 2H), 6.82 (d, J=9.0 Hz, 2H), 6.11 (d, J=5.7 Hz, 1H), 4.08 (s, 2H), 3.95 (t, J=6.3 Hz, 2H), 3.59-3.52 (m, 4H), 2.41 (t, J=7.2 Hz, 2H), 2.36 (s, 4H), 1.91-1.77 (m, 2H), 1.32 (s, 6H).

### Example 153: Preparation of 7-methoxy-5,6,7,8-tetrahydroquinolin-3-amine (Intermediate 59)

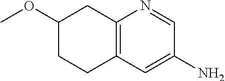

### Step 1: 7-methoxy-3-nitro-5,6,7,8-tetrahydroquinoline

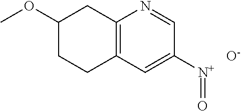

The title compound was prepared from 3-methoxycyclohexanone according to 3-nitropyridine synthesis general procedure R and was purified by flash chromatography. ¹H NMR (400 MHz, cdcl3) δ (ppm): 9.17 (d, J=2.5 Hz, 1H), 8.16 (d, J=2.5 Hz, 1H), 3.87 (m, 1H), 3.41 (s, 3H), 3.22 (dd, J=18.4, 4.6 Hz, 1H), 3.14 (dd, J=18.5, 5.0 Hz, 1H), 3.06 (ddd, J=15.8, 9.2, 5.8 Hz, 1H), 2.84 (dt, J=17.2, 5.9 Hz, 1H), 2.13-2.03 (m, 1H), 2.03-1.93 (m, 1H).

### Step 2: 7-methoxy-5,6,7,8-tetrahydroquinolin-3-amine

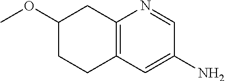

The title compound was prepared from 7-methoxy-3-nitro-5,6,7,8-tetrahydroquinoline according to nitro reduction general procedure I and was purified by flash chromatography. [M+H]⁺: 179.2.

### Example 154: Preparation of Compound 66

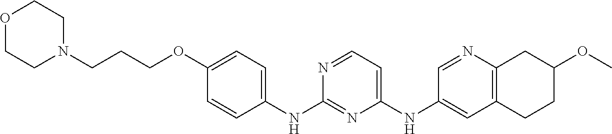 [SMILES: COC1CCc2cc(Nc3ccnc(Nc4ccc(OCCCN5CCOCC5)cc4)n3)cnc2C1]

Compound 66 was prepared from the corresponding intermediate according to general procedure D. MS (m/z): [M+H]⁺ 491.6. ¹H NMR (500 MHz, DMSO) δ 9.34 (s, 1H), 8.97 (s, 1H), 8.46-8.39 (m, 1H), 7.97 (d, J=5.7 Hz, 1H), 7.93 (d, J=2.4 Hz, 1H), 7.51 (d, J=8.7 Hz, 2H), 6.83 (d, J=9.1 Hz, 2H), 6.14 (d, J=5.7 Hz, 1H), 3.96 (t, J=6.4 Hz, 2H), 3.76-3.71 (m, 1H), 3.57 (t, J=4.6 Hz, 4H), 3.31 (s, 3H), 3.02 (dd, J=17.0, 4.6 Hz, 1H), 2.79-2.71 (m, 2H), 2.68-2.57 (m, 1H), 2.41 (t, J=7.2 Hz, 2H), 2.36 (s, 4H), 1.93 (dd, J=16.8, 9.3 Hz, 1H), 1.89-1.77 (m, 3H).

### Example 155: Preparation of 6-methoxy-5,6,7,8-tetrahydroquinolin-3-amine (Intermediate 60)

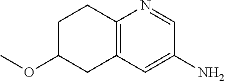

### Step 1: 6-methoxy-3-nitro-5,6,7,8-tetrahydroquinoline

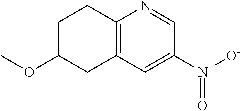

The title compound was prepared from 4-methoxycyclohexanone according to 3-nitropyridine synthesis general procedure R and was purified by flash chromatography. [M+H]⁺: 209.1.

### Step 2: 6-methoxy-5,6,7,8-tetrahydroquinolin-3-amine

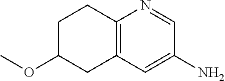

The title compound was prepared from 6-methoxy-3-nitro-5,6,7,8-tetrahydroquinoline according to nitro reduction general procedure I and was purified by flash chromatography. [M+H]⁺: 179.1.

### Example 156: Preparation of Compound 67

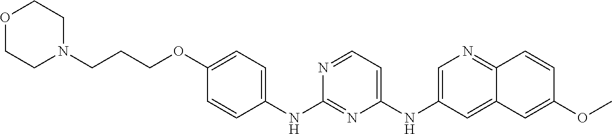 [SMILES: COc1ccc2ncc(Nc3ccnc(Nc4ccc(OCCCN5CCOCC5)cc4)n3)cc2c1]

Compound 67 was prepared from the corresponding intermediate according to general procedure D. MS (m/z): [M+H]⁺ 491.4. ¹H NMR (500 MHz, DMSO) δ 9.33 (s, 1H), 8.96 (s, 1H), 8.40 (d, J=1.1 Hz, 1H), 8.02-7.93 (m, 2H), 7.51 (d, J=8.8 Hz, 2H), 6.84 (d, J=9.0 Hz, 2H), 6.15 (d, J=5.7 Hz, 1H), 3.96 (t, J=6.5 Hz, 2H), 3.72-3.64 (m, 1H), 3.57 (t, J=4.7 Hz, 4H), 2.95 (dd, J=15.9, 4.4 Hz, 1H), 2.83 (dt, J=13.6, 6.2 Hz, 1H), 2.79-2.65 (m, 2H), 2.42 (t, J=7.2 Hz, 2H), 2.39-2.33 (m, 4H), 2.00 (dt, J=10.8, 7.7 Hz, 1H), 1.94-1.82 (m, 3H). A 3H singlet overlaps with the water peak.

### Example 157: Preparation of 7′,8′-dihydro-5′H-spiro[[1.3]dioxolane-2,6′-quinolin]-3′-amine (Intermediate 67)

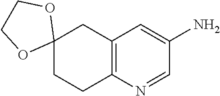 [SMILES: Nc1cnc2c(c1)CC1(CC2)OCCO1]

### Step 1: 3′-nitro-7′,8′-dihydro-5′H-spiro[[1,3]dioxolane-2,6′-quinoline]

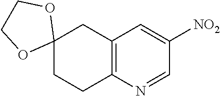 [SMILES: O=[N+]([O-])c1cnc2c(c1)CC1(CC2)OCCO1]

The title compound was prepared from 1,4-cyclohexanedione monoethylene acetal according to 3-nitropyridine synthesis general procedure R and the crude material was used directly in the next step.

### Step 2: 7′,8′-dihydro-5′H-spiro[[1,3]dioxolane-2,6′-quinolin]-3′-amine

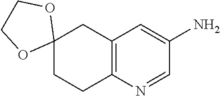 [SMILES: Nc1cnc2c(c1)CC1(CC2)OCCO1]

The title compound was prepared from crude 3′-nitro-7′,8′-dihydro-5′h-spiro[[1,3]dioxolane-2,6′-quinoline]according to nitro reduction general procedure H and was purified by flash chromatography. [M+H]⁺: 207.2.

### Example 158: Preparation of Compound 68

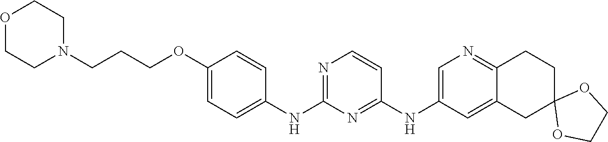 [SMILES: c1cc(Nc2cnc3c(c2)CC2(CC3)OCCO2)nc(Nc2ccc(OCCCN3CCOCC3)cc2)n1]

Compound 68 was prepared from the corresponding intermediate according to general procedure D. MS (m/z): [M+H]⁺ 519.4. H NMR (500 MHz, CD3CN) δ 8.40 (s, 1H), 7.97 (d, J=5.7 Hz, 1H), 7.87 (s, 1H), 7.65 (s, 1H), 7.51-7.41 (m, 3H), 6.86 (d, J=8.8 Hz, 2H), 6.12 (d, J=5.7 Hz, 1H), 4.04-3.93 (m, 6H), 3.61 (t, J=4.4 Hz, 4H), 2.96 (t, J=6.9 Hz, 2H), 2.90 (s, 2H), 2.46 (t, J=7.2 Hz, 2H), 2.40 (s, 4H), 1.99 (t, J=6.8 Hz, 2H), 1.92-1.84 (m, 2H).

### Example 159: Preparation of 7-methyl-5,6,7,8-tetrahydroquinolin-3-amine (Intermediate 62)

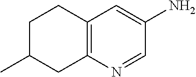

### Step 1: 7-methyl-3-nitro-5,6,7,8-tetrahydroquinoline

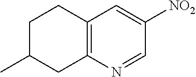

The title compound was prepared from 3-methylcyclohexanone according to 3-nitropyridine synthesis general procedure R and the crude material was used directly in the next step.

### Step 2: 7-methyl-5,6,7,8-tetrahydroquinolin-3-amine

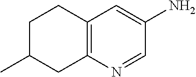

The title compound was prepared from crude 7-methyl-3-nitro-5,6,7,8-tetrahydroquinoline according to nitro reduction general procedure H and was purified by flash chromatography. ¹H NMR (500 MHz, CDCl3) δ (ppm): 7.88 (d, J=2.7 Hz, 1H), 6.70 (d, J=2.7 Hz, 1H), 3.64 (s, 2H), 2.89 (ddd, J=16.7, 5.1, 1.5 Hz, 1H), 2.73-2.65 (m, J=6.2, 4.0 Hz, 2H), 2.38 (dd, J=16.9, 10.6 Hz, 1H), 1.96-1.79 (m, 2H), 1.39-1.30 (m, 1H), 1.06 (d, J=6.5 Hz, 3H).

### Example 160: Preparation of Compound 69

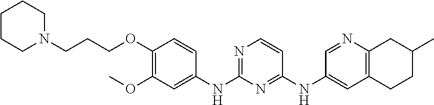 [SMILES: COc1cc(Nc2nccc(Nc3cnc4c(c3)CCC(C)C4)n2)ccc1OCCCN1CCCCC1]

Compound 69 was prepared from the corresponding intermediate according to general procedure D. MS (m/z): [M+H]⁺ 503.5. H NMR (500 MHz, CDCl3) δ 9.31 (s, 1H), 8.93 (s, 1H), 8.45 (s, 1H), 7.99 (s, 1H), 7.90 (d, J=2.0 Hz, 1H), 7.25 (s, 1H), 7.15 (d, J=8.2 Hz, 1H), 6.82 (d, J=8.7 Hz, 1H), 6.15 (d, J=5.7 Hz, 1H), 3.92 (t, J=6.5 Hz, 2H), 3.64-3.56 (m, 3H), 2.85 (dd, J=17.0, 4.7 Hz, 1H), 2.73-2.66 (m, 2H), 2.42-2.31 (m, 7H), 1.92-1.79 (m, 4H), 1.53-1.46 (m, 4H), 1.36 (dd, J=20.3, 10.8 Hz, 3H), 1.05 (d, J=6.6 Hz, 3H).

### Example 161: Preparation of 6,7,8,9-tetrahydro-5H-5,8-methanocyclohepta[b]pyridin-3-amine (Intermediate 63)

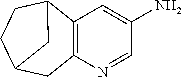 [SMILES: Nc1cnc2c(c1)C1CCC(C2)C1]

### Step 1: 3-nitro-6,7,8,9-tetrahydro-5H-5,8-methanocyclohepta[b]pyridine

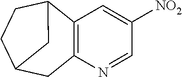 [SMILES: O=[N+]([O-])c1cnc2c(c1)C1CCC(C2)C1]

The title compound was prepared from bicyclo[3.2.1]octan-3-one according to 3-nitropyridine synthesis general procedure R and the crude material was used directly in the next step. [M+H]⁺: 205.2.

### Step 2: 6,7,8,9-tetrahydro-5H-5,8-methanocyclohepta[b]pyridin-3-amine

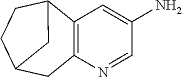 [SMILES: Nc1cnc2c(c1)C1CCC(C2)C1]

The title compound was prepared from crude 3-nitro-6,7,8,9-tetrahydro-5H-5,8-methanocyclohepta[b]pyridine according to nitro reduction general procedure H and was purified by flash chromatography. [M+H]⁺: 175.2.

### Example 162: Preparation of Compound 7065gh

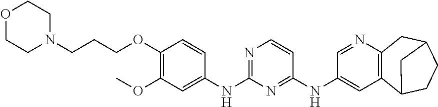 [SMILES: COc1cc(Nc2nccc(Nc3cnc4c(c3)C3CCC(C4)C3)n2)ccc1OCCCN1CCOCC1]

Compound 70 was prepared from the corresponding intermediate according to general procedure D. MS (m/z): [M+H]⁺ 515.5. H NMR (400 MHz, dmso) δ 9.33 (s, 1H), 8.94 (s, 1H), 8.42 (s, 1H), 7.98 (d, J=5.7 Hz, 1H), 7.88 (d, J=2.3 Hz, 1H), 7.25 (s, 1H), 7.19 (dd, J=8.6, 2.1 Hz, 1H), 6.83 (d, J=8.8 Hz, 1H), 6.16 (d, J=5.7 Hz, 1H), 3.92 (t, J=6.4 Hz, 2H), 3.65 (s, 3H), 2.99 (dd, J=17.8, 4.9 Hz, 2H), 2.60 (d, J=16.4 Hz, 2H), 2.37 (m, 6H), 1.98-1.80 (m, 4H), 1.74-1.61 (m, 3H), 1.50 (m, 4H), 1.44-1.34 (m, 3H).

### Example 163: Preparation of (R)-7-methyl-5,6,7,8-tetrahydroquinolin-3-amine (Intermediate 64)

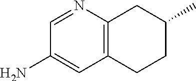

### Step 1: (R)-7-methyl-3-nitro-5,6,7,8-tetrahydroquinoline

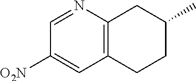

The title compound was prepared from (R)-(+)-3-methylcyclohexanone according to 3-nitropyridine synthesis general procedure R and the crude material was used directly in the next step.

### Step 2: (R)-7-methyl-5,6,7,8-tetrahydroquinolin-3-amine

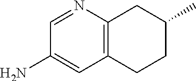

The title compound was prepared from crude (R)-7-methyl-3-nitro-5,6,7,8-tetrahydroquinoline according to nitro reduction general procedure H and was purified by flash chromatography. [M+H]⁺: 163.1.

### Example 164: Preparation of Compound 71

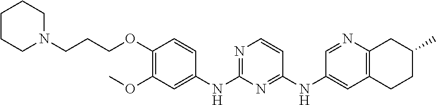 [SMILES: COc1cc(Nc2nccc(Nc3cnc4c(c3)CC[C@@H](C)C4)n2)ccc1OCCCN1CCCCC1]

Compound 71 was prepared from the corresponding intermediate according to general procedure D. MS (m/z): [M+H]⁺ 503.5. ¹H NMR (500 MHz, DMSO) δ 9.31 (s, 1H), 8.94 (s, 1H), 8.45 (s, 1H), 7.99 (d, J=5.7 Hz, 1H), 7.90 (d, J=2.4 Hz, 1H), 7.25 (s, 1H), 7.15 (d, J=9.0 Hz, 1H), 6.82 (d, J=8.7 Hz, 1H), 6.15 (d, J=5.7 Hz, 1H), 3.92 (t, J=6.4 Hz, 2H), 3.62 (s, 3H), 2.85 (dd, J=16.5, 5.0 Hz, 1H), 2.73-2.65 (m, 2H), 2.44-2.26 (m, 7H), 1.93-1.77 (m, 4H), 1.50 (s, 4H), 1.44-1.28 (m, 3H), 1.05 (d, J=6.6H, 3H).

### Example 165: Preparation of 8-(tert-butyl)-5,6,7,8-tetrahydroquinolin-3-amine (Intermediate 65)

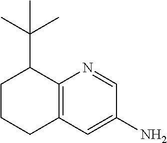

### Step 1: 8-(tert-butyl)-3-nitro-5,6,7,8-tetrahydroquinoline

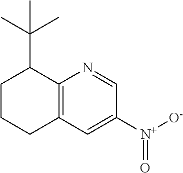 [SMILES: CC(C)(C)C1CCCc2cc([N+](=O)[O-])cnc21]

The title compound was prepared from 2-tert-butylcyclohexanone according to 3-nitropyridine synthesis general procedure R and the crude material was used directly in the next step.

### Step 2: 8-(tert-butyl)-5,6,7,8-tetrahydroquinolin-3-amine

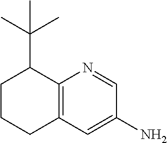

The title compound was prepared from crude 8-(tert-butyl)-3-nitro-5,6,7,8-tetrahydroquinoline according to nitro reduction general procedure H and was purified by flash chromatography. [M+H]⁺: 205.2.

### Example 167: Preparation of 8,8-dimethyl-5,6,7,8-tetrahydroquinolin-3-amine (Intermediate 66)

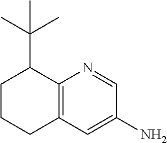

### Step 1: 8,8-dimethyl-3-nitro-5,6,7,8-tetrahydroquinoline

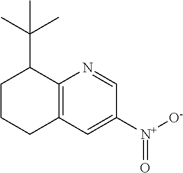 [SMILES: CC(C)(C)C1CCCc2cc([N+](=O)[O-])cnc21]

The title compound was prepared from 2,2-dimethylcyclohexanone according to 3-nitropyridine synthesis general procedure R and the crude material was used directly in the next step.

### Step 2: 8,8-dimethyl-5,6,7,8-tetrahydroquinolin-3-amine

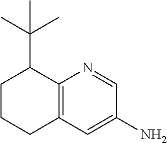

The title compound was prepared from crude 8,8-dimethyl-3-nitro-5,6,7,8-tetrahydroquinoline according to nitro reduction general procedure H and was purified by flash chromatography. [M+H]⁺: 176.9.

### Example 168: Preparation of Compound 73

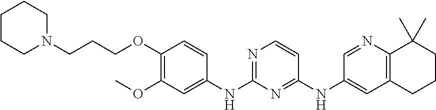 [SMILES: COc1cc(Nc2nccc(Nc3cnc4c(c3)CCCC4(C)C)n2)ccc1OCCCN1CCCCC1]

Compound 73 was prepared from the corresponding intermediate according to general procedure D. MS (m/z): [M+H]⁺ 517.5. H NMR (500 MHz, DMSO) δ 9.35 (s, 1H), 8.99 (s, 1H), 8.68 (s, 1H), 7.99 (d, J=5.7 Hz, 1H), 7.74 (d, J=2.0 Hz, 1H), 7.33 (s, 1H), 7.20 (d, J=7.6 Hz, 1H), 6.85 (d, J=8.7 Hz, 1H), 6.18 (d, J=5.7 Hz, 1H), 3.96 (t, J=5.8 Hz, 2H), 3.65 (s, 3H), 3.20-2.55 (br m, 4H), 2.87 (t, J=5.69 Hz, 2H), 2.03 (broad s, 2H), 1.86-1.32 (m, 12H), 1.25 (s, 6H).

### Example 169: Preparation of 6′,7′-dihydro-5′H-spiro[cyclopentane-1,8′-quinolin]-3′-amine (Intermediate 67)

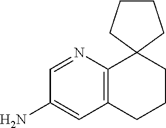 [SMILES: Nc1cnc2c(c1)CCCC21CCCC1]

### Step 1: 3′-nitro-6′,7′-dihydro-5′H-spiro[cyclopentane-1,8′-quinoline]

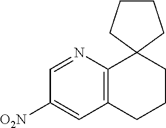 [SMILES: O=[N+]([O-])c1cnc2c(c1)CCCC21CCCC1]

The title compound was prepared from spiro[4.5]decan-6-one according to 3-nitropyridine synthesis general procedure R and the crude material was used directly in the next step.

### Step 2: 6′,7′-dihydro-5′H-spiro[cyclopentane-1,8′-quinolin]-3′-amine

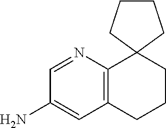 [SMILES: Nc1cnc2c(c1)CCCC21CCCC1]

The title compound was prepared from crude 3′-nitro-6′,7′-dihydro-5′h-spiro[cyclopentane-1,8′-quinoline]according to nitro reduction general procedure H and was purified by flash chromatography. [M+H]⁺: 203.2.

### Example 170: Preparation of Compound 74

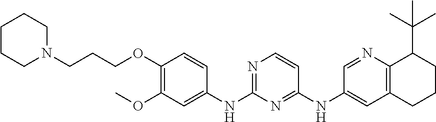 [SMILES: COc1cc(Nc2nccc(Nc3cnc4c(c3)CCCC4C(C)(C)C)n2)ccc1OCCCN1CCCCC1]

Compound 74 was prepared from the corresponding intermediate according to general procedure D. MS (m/z): [M+H]⁺ 545.6. ¹H NMR (400 MHz, DMSO) δ (ppm): 9.95 (s, 1H), 9.12 (s, 1H), 9.01 (s, 1H), 8.92 (s, 1H), 8.17 (d, J=2.1 Hz, 1H), 8.11 (d, J=5.7 Hz, 1H), 7.99-7.89 (m, 1H), 7.74 (s, 1H), 7.51 (s, 1H), 6.32 (d, J=5.7 Hz, 1H), 4.32 (s, 1H), 3.76 (d, J=11.4 Hz, 1H), 3.72-3.64 (m, 4H), 3.00 (d, J=11.3 Hz, 1H), 2.88-2.81 (m, 1H), 2.63-2.58 (m, 2H), 2.40 (t, J=11.0 Hz, 1H), 2.09 (s, 1H), 1.13 (s, 3H), 1.11 (s, 3H).

### Example 171: Preparation of 5,6,7,8-tetrahydro-5,8-ethanoquinolin-3-amine (Intermediate 68)

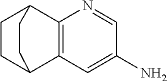 [SMILES: Nc1cnc2c(c1)C1CCC2CC1]

### Step 1: 3-nitro-5,6,7,8-tetrahydro-5,8-ethanoquinoline

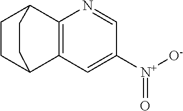 [SMILES: O=[N+]([O-])c1cnc2c(c1)C1CCC2CC1]

The title compound was prepared from bicyclo[2.2.2]octan-2-one according to 3-nitropyridine synthesis general procedure R and the crude material was used directly in the next step.

### Step 2: 5,6,7,8-tetrahydro-5,8-ethanoquinolin-3-amine

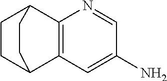 [SMILES: Nc1cnc2c(c1)C1CCC2CC1]

The title compound was prepared from crude 3-nitro-5,6,7,8-tetrahydro-5,8-ethanoquinoline according to nitro reduction general procedure H and was purified by flash chromatography. [M+H]⁺: 175.2.

### Example 172: Preparation of Compound 75

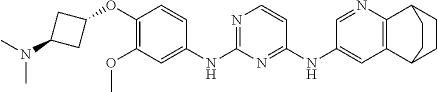 [SMILES: COc1cc(Nc2nccc(Nc3cnc4c(c3)C3CCC4CC3)n2)ccc1O[C@H]1C[C@H](N(C)C)C1]

Compound 75 was prepared from the corresponding intermediate according to general procedure D. MS (m/z): [M+H]⁺ 487.4. ¹H NMR (400 MHz, CD₃CN) δ (ppm): 8.37 (d, J=2.5 Hz, 1H), 7.98 (d, J=5.7 Hz, 1H), 7.93 (d, J=2.5 Hz, 1H), 7.62 (s, 1H), 7.46 (s, 1H), 7.23 (d, J=2.3 Hz, 1H), 7.10 (dd, J=8.7, 2.4 Hz, 1H), 6.65 (d, J=8.6 Hz, 1H), 6.13 (d, J=5.7 Hz, 1H), 4.76-4.59 (m, 1H), 3.71 (s, 3H), 3.06-3.03 (m, 1H), 3.01-2.97 (m, 1H), 2.92-2.80 (m, 1H), 2.37-2.28 (m, 2H), 2.22-2.19 (m, 2H), 2.12 (s, 6H), 1.90-1.77 (m, 4H), 1.47-1.29 (m, 4H).

### Example 173: Preparation of 5,6,7,8-tetrahydro-5,8-methanoquinolin-3-amine (Intermediate 69)

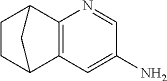 [SMILES: Nc1cnc2c(c1)C1CCC2C1]

### Step 1: 3-nitro-5,6,7,8-tetrahydro-5,8-methanoquinoline

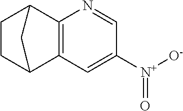 [SMILES: O=[N+]([O-])c1cnc2c(c1)C1CCC2C1]

The title compound was prepared from bicyclo[2.2.2]octan-2-one according to 3-nitropyridine synthesis general procedure R and the crude material was used directly in the next step.

### Step 2: 5,6,7,8-tetrahydro-5,8-methanoquinolin-3-amine

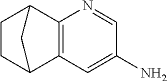 [SMILES: Nc1cnc2c(c1)C1CCC2C1]

The title compound was prepared from crude 3-nitro-5,6,7,8-tetrahydro-5,8-methanoquinoline according to nitro reduction general procedure H and was purified by flash chromatography. [M+H]⁺: 161.1.

### Example 174: Preparation of Compound 76

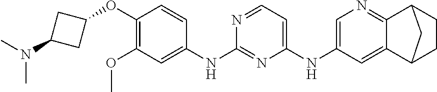 [SMILES: COc1cc(Nc2nccc(Nc3cnc4c(c3)C3CCC4C3)n2)ccc1O[C@H]1C[C@H](N(C)C)C1]

Compound 76 was prepared from the corresponding intermediate according to general procedure D. MS (m/z): [M+H]⁺ 473.4. ¹H NMR (500 MHz, DMSO) δ 9.28 (s, 1H), 8.93 (s, 1H), 8.24 (s, 1H), 8.04 (d, J=2.2 Hz, 1H), 7.98 (d, J=5.7 Hz, 1H), 7.27 (s, 1H), 7.22-7.12 (m, 1H), 6.62 (d, J=8.7 Hz, 1H), 6.14 (d, J=5.8 Hz, 1H), 4.67-4.58 (m, 1H), 3.66 (s, 3H), 3.36 (s, 1H), 3.26 (s, 1H), 2.83 (s, 1H), 2.32-2.24 (m, 2H), 2.17-2.11 (m, 2H), 2.07 (s, 6H), 1.95 (d, J=7.4 Hz, 2H), 1.75-1.71 (m, 1H), 1.59-1.54 (m, 1H), 1.18-1.10 (m, 2H).

### Example 175: Preparation of 6′,8′-dihydro-5′H-spiro[cyclobutane-1,7′-quinolin]-3′-amine (Intermediate

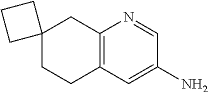 [SMILES: Nc1cnc2c(c1)CCC1(CCC1)C2]

### Step 1: 3′-nitro-6′,8′-dihydro-5′H-spiro[cyclobutane-1,7′-quinoline]

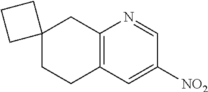 [SMILES: O=[N+]([O-])c1cnc2c(c1)CCC1(CCC1)C2]

The title compound was prepared from spiro[3.5]nonan-6-one according to 3-nitropyridine synthesis general procedure R and the crude material was used directly in the next step.

### Step 2: 6′,8′-dihydro-5′H-spiro[cyclobutane-1,7′-quinolin]-3′-amine

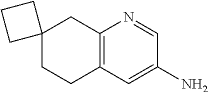 [SMILES: Nc1cnc2c(c1)CCC1(CCC1)C2]

The title compound was prepared from crude 3′-nitro-6′,8′-dihydro-5′h-spiro[cyclobutane-1,7′-quinoline]according to nitro reduction general procedure H and was purified by flash chromatography. ¹H NMR (400 MHz, CDCl3) δ (ppm): 7.90 (d, J=2.7 Hz, 1H), 6.70 (d, J=2.7 Hz, 1H), 3.48 (broad s, 2H), 2.86 (s, 2H), 2.69 (t, J=6.5 Hz, 2H), 2.10-1.62 (m, 8H).

### Example 176: Preparation of Compound 77

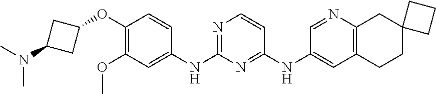 [SMILES: COc1cc(Nc2nccc(Nc3cnc4c(c3)CCC3(CCC3)C4)n2)ccc1O[C@H]1C[C@H](N(C)C)C1]

Compound 77 was prepared from the corresponding intermediate according to general procedure D. MS (m/z): [M+H]⁺ 501.4. H NMR (500 MHz, DMSO) δ 9.32 (s, 1H), 8.93 (s, 1H), 8.42 (s, 1H), 7.98 (d, J=5.7 Hz, 1H), 7.91 (d, J=2.4 Hz, 1H), 7.22 (s, 1H), 7.16 (dd, J=8.7, 1.7 Hz, 1H), 6.62 (d, J=8.7 Hz, 1H), 6.15 (d, J=5.8 Hz, 1H), 4.66-4.59 (m, 1H), 3.62 (s, 3H), 2.82 (s, 2H), 2.67 (t, J=6.1 Hz, 2H), 2.33-2.24 (m, 2H), 2.19-2.07 (m, 9H), 1.97-1.81 (m, 4H), 1.79-1.73 (m, 4H).

### Example 177: Preparation of 6′,8′-dihydro-5′H-spiro[cyclopentane-1,7′-quinolin]-3′-amine (Intermediate

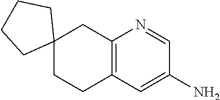 [SMILES: Nc1cnc2c(c1)CCC1(CCCC1)C2]

### Step 1: 3′-nitro-6′,8′-dihydro-5′H-spiro[cyclopentane-1,7′-quinoline]

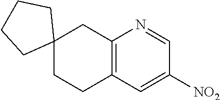 [SMILES: O=[N+]([O-])c1cnc2c(c1)CCC1(CCCC1)C2]

The title compound was prepared from spiro[4.5]decan-7-one according to 3-nitropyridine synthesis general procedure R and the crude material was used directly in the next step.

### Step 2: 6′,8′-dihydro-5′H-spiro[cyclopentane-1,7′-quinolin]-3′-amine

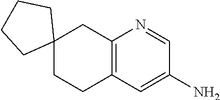 [SMILES: Nc1cnc2c(c1)CCC1(CCCC1)C2]

The title compound was prepared in 74% yield from crude 3′-nitro-6′,8′-dihydro-5′h-spiro[cyclopentane-1,7′-quinoline]according to nitro reduction general procedure H and was purified by flash chromatography. ¹H NMR (500 MHz, CDCl3) δ (ppm): 7.98 (d, J=2.0 Hz, 1H), 6.85 (d, J=0.9 Hz, 1H), 3.86 (s, 2H), 2.93-2.61 (m, 4H), 1.91-1.57 (m, 6H), 1.46 (m, 4H).

### Example 178: Preparation of Compound 78

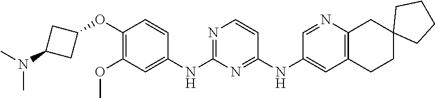 [SMILES: COc1cc(Nc2nccc(Nc3cnc4c(c3)CCC3(CCCC3)C4)n2)ccc1O[C@H]1C[C@H](N(C)C)C1]

Compound 78 was prepared from the corresponding intermediate according to general procedure D. MS (m/z): [M+H]⁺ 515.5. H NMR (500 MHz, DMSO) δ 9.39 (s, 1H), 9.00 (s, 1H), 8.47 (s, 1H), 8.05 (d, J=5.8 Hz, 1H), 8.01 (d, J=2.0 Hz, 1H), 7.39-7.25 (m, 1H), 7.23 (dd, J=8.5, 2.0 Hz, 1H), 6.69 (d, J=8.7 Hz, 1H), 6.22 (d, J=5.8 Hz, 1H), 4.75-4.64 (m, 1H), 3.69 (s, 3H), 2.90 (s, 1H), 2.76 (t, J=6.1 Hz, 2H), 2.70 (s, 2H), 2.34 (s, 2H), 2.26-2.05 (m, 8H), 1.78-1.62 (m, 6H), 1.56-1.37 (m, 4H).

### Example 179: Preparation of 7′,8′-dihydro-5′H-spiro[cyclopentane-1,6′-quinolin]-3′-amine (Intermediate 72)

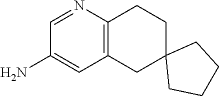 [SMILES: Nc1cnc2c(c1)CC1(CCCC1)CC2]

### Step 1: 3′-nitro-7′,8′-dihydro-5′H-spiro[cyclopentane-1,6′-quinoline]

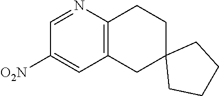 [SMILES: O=[N+]([O-])c1cnc2c(c1)CC1(CCCC1)CC2]

The title compound was prepared from spiro[4.5]decan-8-one according to 3-nitropyridine synthesis general procedure R and the crude material was used directly in the next step.

### Step 2: 7′,8′-dihydro-5′H-spiro[cyclopentane-1,6′-quinolin]-3′-amine

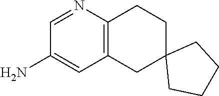 [SMILES: Nc1cnc2c(c1)CC1(CCCC1)CC2]

The title compound was prepared from crude 3′-nitro-7′,8′-dihydro-5′h-spiro[cyclopentane-1,6′-quinoline]according to nitro reduction general procedure H and was purified by flash chromatography. [M+H]⁺: 203.2.

### Example 180: Preparation of Compound 79

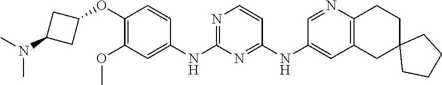 [SMILES: COc1cc(Nc2nccc(Nc3cnc4c(c3)CC3(CCCC3)CC4)n2)ccc1O[C@H]1C[C@H](N(C)C)C1]

Compound 79 was prepared from the corresponding intermediate according to general procedure D. MS (m/z): [M+H]⁺ 515.3. H NMR (500 MHz, DMSO) δ 9.32 (s, 1H), 8.93 (s, 1H), 8.42 (s, 1H), 7.99 (d, J=5.8 Hz, 1H), 7.93 (d, J=2.4 Hz, 1H), 7.25 (s, 1H), 7.12 (d, J=7.1 Hz, 1H), 6.63 (d, J=8.7 Hz, 1H), 6.16 (d, J=5.7 Hz, 1H), 4.78-4.53 (m, 1H), 3.63 (s, 3H), 2.82 (s, 1H), 2.79 (t, J=6.5 Hz, 2H), 2.53 (s, 2H), 2.33-2.24 (m, 2H), 2.17-2.04 (m, 8H), 1.72 (t, J=6.5 Hz, 2H), 1.67 (dd, J=9.3, 4.9 Hz, 4H), 1.50-1.42 (m, 2H), 1.42-1.34 (m, 2H).

### Example 181: Preparation of 7′,8′-dihydro-5′H-spiro[cyclobutane-1,6′-quinolin]-3′-amine (Intermediate

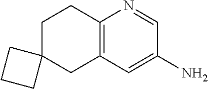 [SMILES: Nc1cnc2c(c1)CC1(CCC1)CC2]

### Step 1: 3′-nitro-7′,8′-dihydro-5′H-spiro[cyclobutane-1,6′-quinoline]

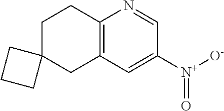 [SMILES: O=[N+]([O-])c1cnc2c(c1)CC1(CCC1)CC2]

The title compound was prepared from spiro[3.5]nonan-7-one according to 3-nitropyridine synthesis general procedure R and the crude material was used directly in the next step.

### Step 2: 7′,8′-dihydro-5′H-spiro[cyclobutane-1,6′-quinolin]-3′-amine

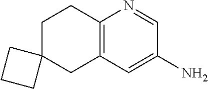 [SMILES: Nc1cnc2c(c1)CC1(CCC1)CC2]

The title compound was prepared from crude 3′-nitro-7′,8′-dihydro-5′h-spiro[cyclobutane-1,6′-quinoline]according to nitro reduction general procedure H and was purified by flash chromatography. [M+H]⁺: 189.2.

### Example 182: Preparation of Compound 80

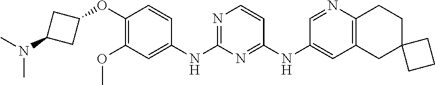 [SMILES: COc1cc(Nc2nccc(Nc3cnc4c(c3)CC3(CCC3)CC4)n2)ccc1O[C@H]1C[C@H](N(C)C)C1]

Compound 80 was prepared from the corresponding intermediate according to general procedure D. MS (m/z): [M+H]⁺ 501.4. H NMR (500 MHz, DMSO) δ 9.33 (s, 1H), 8.94 (s, 1H), 8.49-8.31 (m, 1H), 7.99 (d, J=5.7 Hz, 1H), 7.98 (d, J=2.4 Hz, 1H), 7.25 (s, 1H), 7.14 (d, J=8.4 Hz, 1H), 6.65 (d, J=8.8 Hz, 1H), 6.16 (d, J=5.7 Hz, 1H), 4.69-4.60 (m, 1H), 3.64 (s, 3H), 2.83 (s, 1H), 2.77 (t, J=6.5 Hz, 2H), 2.71 (s, 2H), 2.32-2.25 (m, 2H), 2.18-2.10 (m, 2H), 2.08 (s, 6H), 2.01-1.81 (m, 6H), 1.79-1.67 (m, 2H).

### Example 183: Preparation of 6,6-difluoro-5,6,7,8-tetrahydroquinolin-3-amine (Intermediate 74)

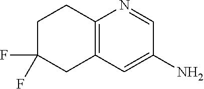 [SMILES: Nc1cnc2c(c1)CC(F)(F)CC2]

### Step 1: 6,6-difluoro-3-nitro-5,6,7,8-tetrahydroquinoline

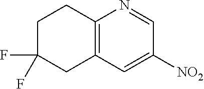 [SMILES: O=[N+]([O-])c1cnc2c(c1)CC(F)(F)CC2]

The title compound was prepared from 4,4-difluorocyclohexanone according to 3-nitropyridine synthesis general procedure R and the crude material was used directly in the next step.

### Step 2: 6,6-difluoro-5,6,7,8-tetrahydroquinolin-3-amine

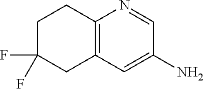 [SMILES: Nc1cnc2c(c1)CC(F)(F)CC2]

The title compound was prepared from crude 6,6-difluoro-3-nitro-5,6,7,8-tetrahydroquinoline according to nitro reduction general procedure H and was purified by flash chromatography. ¹H NMR (400 MHz, cdcl3) δ (ppm): 7.94 (d, J=2.5 Hz, 1H), 6.69 (d, J=2.5 Hz, 1H), 3.66 (s, 2H), 3.16 (t, J=14.5 Hz, 2H), 3.04 (t, J=6.9 Hz, 2H), 2.26 (tt, J=13.9, 7.0 Hz, 2H).

### Example 184: Preparation of Compound 81

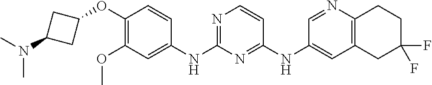 [SMILES: COc1cc(Nc2nccc(Nc3cnc4c(c3)CC(F)(F)CC4)n2)ccc1O[C@H]1C[C@H](N(C)C)C1]

Compound 81 was prepared from the corresponding intermediate according to general procedure D. MS (m/z): [M+H]⁺ 497.3. ¹H NMR (400 MHz, dmso) δ 9.47 (s, 1H), 8.96 (s, 1H), 8.51 (s, 1H), 8.08 (d, J=2.1 Hz, 1H), 8.01 (d, J=5.7 Hz, 1H), 7.24-7.09 (m, 2H), 6.64 (d, J=8.6 Hz, 1H), 6.18 (d, J=5.7 Hz, 1H), 4.69-4.55 (m, 1H), 3.66 (s, 3H), 3.24 (d, J=14.6 Hz, 2H), 2.98 (t, J=6.9 Hz, 2H), 2.80 (m, 1H), 2.35-2.25 (m, 4H), 2.17-2.08 (m, 2H), 2.06 (s, 6H).

### Example 185: Preparation of 3-amino-5,6,7,8-tetrahydroquinolin-6-ol (Intermediate 75)

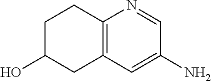 [SMILES: Nc1cnc2c(c1)CC(O)CC2]

### Step 1: 3-nitro-5,6,7,8-tetrahydroquinolin-6-ol

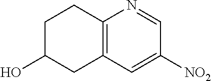 [SMILES: O=[N+]([O-])c1cnc2c(c1)CC(O)CC2]

The title compound was prepared from 4-hydroxycyclohexanone according to 3-nitropyridine synthesis general procedure R and was purified by flash chromatography. [M+H]⁺: 195.1.

### Step 2: 3-amino-5,6,7,8-tetrahydroquinolin-6-ol

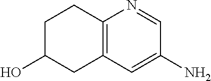 [SMILES: Nc1cnc2c(c1)CC(O)CC2]

The title compound was prepared from 3-nitro-5,6,7,8-tetrahydroquinolin-6-ol according to nitro reduction general procedure H and the crude material was used directly in the next step. [M+H]⁺: 165.1.

### Example 186: Preparation of Compound 82

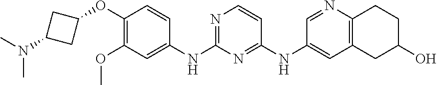 [SMILES: COc1cc(Nc2nccc(Nc3cnc4c(c3)CC(O)CC4)n2)ccc1O[C@H]1C[C@@H](N(C)C)C1]

Compound 82 was prepared from the corresponding intermediate according to general procedure C. MS (m/z): [M+H]⁺ 477.5. H NMR (500 MHz, DMSO) δ 9.38 (s, 1H), 9.02 (s, 1H), 8.53 (s, 1H), 8.05 (d, J=5.7 Hz, 1H), 7.96 (s, 1H), 7.31 (s, 1H), 7.27 (d, J=8.9 Hz, 1H), 6.79 (d, J=8.7 Hz, 1H), 6.22 (d, J=5.7 Hz, 1H), 4.89 (s, 1H), 4.43-4.34 (m, 1H), 4.04 (m, 1H), 3.70 (s, 3H), 3.35 (s, 1H), 2.94 (dd, J=14.9, 8.9 Hz, 2H), 2.86-2.75 (m, 1H), 2.69-2.59 (m, 3H), 2.14 (s, 6H), 2.05-1.78 (m, 4H).

### Example 187: Preparation of 4-((1s,3s)-3-(dimethylamino)cyclobutoxy)-3-methoxyaniline (Intermediate

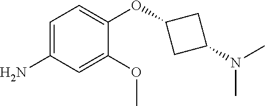 [SMILES: COc1cc(N)ccc1O[C@H]1C[C@@H](N(C)C)C1]

### Step 1: (1s,3s)-3-(2-methoxy-4-nitrophenoxy)-N,N-dimethylcyclobutanamine

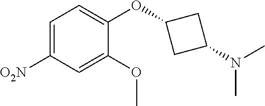 [SMILES: COc1cc([N+](=O)[O-])ccc1O[C@H]1C[C@@H](N(C)C)C1]

The title compound was prepared from (1s,3s)-3-(dimethylamino)cyclobutanol hydrochloride and 1-fluoro-2-methoxy-4-nitrobenzene according to general procedure G and the crude material was used directly in the next step. [M+H]⁺: 267.2.

### Step 2: 4-((1s,3s)-3-(dimethylamino)cyclobutoxy)-3-methoxyaniline

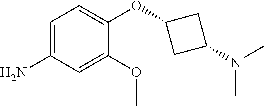 [SMILES: COc1cc(N)ccc1O[C@H]1C[C@@H](N(C)C)C1]

The title compound was prepared from (1s,3s)-3-(2-methoxy-4-nitrophenoxy)-N,N-dimethylcyclobutanamine according to nitro reduction general procedure H and the crude material was used directly in the next step. [M+H]⁺: 237.2.

### Synthesis of 6-chloro-N-(2-chloropyrimidin-4-yl)-1,8-naphthyridin-3-amine

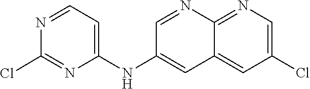 [SMILES: Clc1cnc2ncc(Nc3ccnc(Cl)n3)cc2c1]

### Step 1: 3-bromo-6-chloro-1,8-naphthyridine

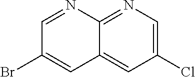 [SMILES: Clc1cnc2ncc(Br)cc2c1]

The title compound was prepared from 3-chloro-1,8-naphthyridine according to Bromination general procedure W and the crude material was used directly in the next step. [M+H]⁺: 242.9, 244.9, 247.0.

### Step 2: tert-butyl (6-chloro-1,8-naphthyridin-3-yl)carbamate

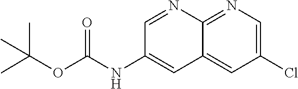 [SMILES: CC(C)(C)OC(=O)Nc1cnc2ncc(Cl)cc2c1]

The title compound was prepared from crude 3-bromo-6-chloro-1,8-naphthyridine according to coupling general procedure L and was purified by flash chromatography. [M+H]⁺: 280.1.

### Step 3: 6-chloro-1,8-naphthyridin-3-amine

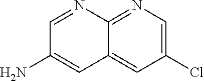 [SMILES: Nc1cnc2ncc(Cl)cc2c1]

The title compound was prepared from tert-butyl (6-chloro-1,8-naphthyridin-3-yl)carbamate according to Boc deprotection general procedure M and was purified by flash chromatography. [M+H]⁺: 180.1.

### Step 4: 6-chloro-N-(2-chloropyrimidin-4-yl)-1,8-naphthyridin-3-amine

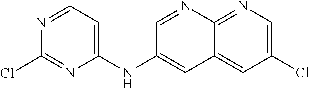 [SMILES: Clc1cnc2ncc(Nc3ccnc(Cl)n3)cc2c1]

The title compound was prepared from 6-chloro-1,8-naphthyridin-3-amine according to general procedure B and was purified by flash chromatography. [M+H]⁺: 292.1.

### Example 188: Preparation of Compound 83

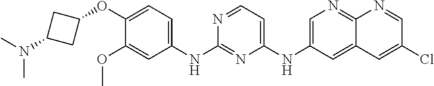 [SMILES: COc1cc(Nc2nccc(Nc3cnc4ncc(Cl)cc4c3)n2)ccc1O[C@H]1C[C@@H](N(C)C)C1]

Compound 83 was prepared from the corresponding intermediate according to general procedure E. MS (m/z): [M+H]⁺ 492.2. ¹H NMR (500 MHz, CD3CN) δ 8.91 (d, J=2.8 Hz, 1H), 8.84 (d, J=2.9 Hz, 1H), 8.73 (d, J=2.6 Hz, 1H), 8.20 (s, 1H), 8.03 (d, J=5.7 Hz, 1H), 8.00 (s, 1H), 7.55 (s, 1H), 7.17 (s, 1H), 7.04 (dd, J=8.5, 2.4 Hz, 1H), 6.73 (d, J=8.6 Hz, 1H), 6.21 (d, J=5.7 Hz, 1H), 4.33 (p, J=7.2 Hz, 1H), 3.62 (s, 3H), 2.57 (dt, J=9.4, 6.6 Hz, 2H), 2.37-2.24 (m, 1H), 2.04 (s, 6H). 2 protons overlap with the solvent.

### Example 189: Preparation of 8-chloro-1,6-naphthyridin-3-amine (Intermediate 77)

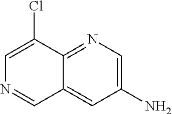 [SMILES: Nc1cnc2c(Cl)cncc2c1]

### Step 1: tert-butyl (3-chloropyridin-4-yl)carbamate

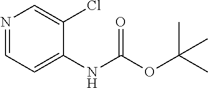

To a solution of 4-Amino-3-chloropyridine (2.00 g, 15.1 mmol) dissolved in DCM (75.5 mL) was added triethylamine (3.17 mL, 22.6 mmol), di-tert-butyl dicarbonate (4.94 g, 22.6 mmol) and DMAP (188 mg, 1.51 mmol). The mixture was stirred overnight at room temperature then quenched with sat. NH₄Cl and extracted three times with DCM. The combined organic layers were dried over Na₂SO₄, filtered and concentrated then the residue was purified by Flash chromatography using 0 to 100% EtOAc in hexanes to give the title compound (2.92 g, 85%) as a white solid. [M-tBu+H]−: 173.0, 175.0.

### Step 2: tert-butyl (3-chloro-5-formylpyridin-4-yl)carbamate

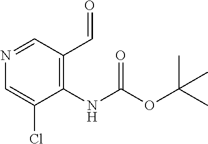 [SMILES: CC(C)(C)OC(=O)Nc1c(Cl)cncc1C=O]

A solution of tert-butyl (2-chloropyridin-3-yl)carbamate (229 mg, 1.00 mmol) and N,N,N′,N′-tetramethylethylenediamine (333 uL, 2.20 mmol) in THF (5 mL) was cooled −78° C. then n-butyllithium (880 uL, 2.20 mmol) was added dropwise. The mixture was warmed to −20° C. and stirred for 1 hour. After cooling back to −78° C. DMF (310 uL, 4.00 mmol) was added then the reaction was removed from the cooling bath and stirred for 30 minutes. Then the reaction was quenched with NH4Cl, filtered and concentrated and the residue was purified by flash chromatography using 0 to 50% EtOAC in hexanes to give the title compound (65.0 mg, 25%) as a yellow gum. [M−H]−: 255.1.

### Step 3: 8-chloro-3-nitro-1,6-naphthyridine

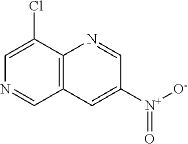 [SMILES: O=[N+]([O-])c1cnc2c(Cl)cncc2c1]

The title compound was prepared from tert-butyl (3-chloro-5-formylpyridin-4-yl)carbamate according to 3-nitronapthiridine synthesis general procedure S and was purified by flash chromatography. H NMR (500 MHz, CDCl3) δ 9.86 (d, J=2.5 Hz, 1H), 9.36 (s, 1H), 9.16 (d, J=2.5 Hz, 1H), 9.01 (s, 1H).

### Step 4: 8-chloro-1,6-naphthyridin-3-amine

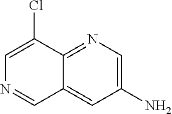 [SMILES: Nc1cnc2c(Cl)cncc2c1]

The title compound was prepared from 8-chloro-3-nitro-1,6-naphthyridine according to nitro reduction general procedure I and was purified by flash chromatography. [M+H]⁺: 180.

### Example 190: Preparation of Compound 84

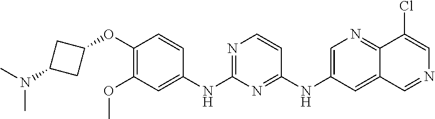 [SMILES: COc1cc(Nc2nccc(Nc3cnc4c(Cl)cncc4c3)n2)ccc1O[C@H]1C[C@@H](N(C)C)C1]

Compound 84 was prepared from the corresponding intermediate according to general procedure D. MS (m/z): [M+H]⁺ 492.6. ¹H NMR (500 MHz, CDCl3) δ 10.21 (s, 1H), 9.27 (s, 1H), 9.22 (s, 1H), 9.18 (s, 1H), 9.07 (s, 1H), 8.71 (s, 1H), 8.14 (d, J=5.7 Hz, 1H), 7.26 (s, 1H), 7.21 (d, J=7.1 Hz, 1H), 6.80 (d, J=8.6 Hz, 1H), 6.34 (d, J=5.6 Hz, 1H), 4.35 (p, J=7.0 Hz, 1H), 3.67 (s, 3H), 2.63-2.56 (m, 2H), 2.40-2.33 (m, 1H), 2.10 (s, 6H), 1.92-1.82 (m, 2H).

### Example 191: Preparation of 7-fluoro-1,5-naphthyridin-3-amine (Intermediate 78)

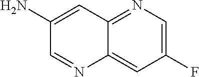 [SMILES: Nc1cnc2cc(F)cnc2c1]

### Step 1: 3-amino-5-fluoropicolinaldehyde

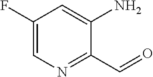

A solution of n-butyllithium in hexanes (5.24 mL, 13.1 mmol) was diluted in THF (28.0 mL) and cooled to −78° C. Then a solution of 3-Amino-2-bromo-5-fluoropyridine (1.02 g, 5.24 mmol) in THF (7.00 mL) was added dropwise. After 1 hour DMF (1.62 mL, 20.9 mmol) was added and the mixture was stirred at −78° C. for an additional 1 hour before quenching with sat. NaHCO₃. The mixture was extracted three times with DCM and the combined organic layers were dried over MgSO₄, filtered and concentrated. The residue was purified by flash chromatography using 0 to 100% DCM in hexanes to give the title compound (198 mg, 27%) as a yellow solid. [M−H]−: 138.9.

### Step 2: 3-fluoro-7-nitro-1,5-naphthyridine

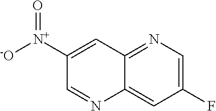

The title compound was prepared from 3-amino-5-fluoropicolinaldehyde according to 3-nitronapthiridine synthesis general procedure S and was purified by flash chromatography. [M+H]⁺: 194.8.

### Step 3: 7-fluoro-1,5-naphthyridin-3-amine

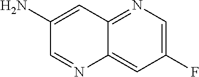 [SMILES: Nc1cnc2cc(F)cnc2c1]

The title compound was prepared from 3-fluoro-7-nitro-1,5-naphthyridine according to nitro reduction general procedure I and was purified by flash chromatography. [M+H]⁺: 164.1.

### Example 192: Preparation of Compound 85

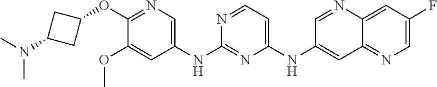 [SMILES: COc1cc(Nc2nccc(Nc3cnc4cc(F)cnc4c3)n2)cnc1O[C@H]1C[C@@H](N(C)C)C1]

Compound 85 was prepared from the corresponding intermediate according to general procedure C. MS (m/z): [M+H]⁺ 477.3. H NMR (500 MHz, DMSO) δ 10.04 (s, 1H), 9.27 (s, 1H), 9.19 (d, J=2.5 Hz, 1H), 9.00 (d, J=2.8 Hz, 1H), 8.96 (s, 1H), 8.21 (dd, J=9.5, 2.3 Hz, 1H), 8.13 (d, J=5.7 Hz, 1H), 7.99 (s, 1H), 7.67 (s, 1H), 6.36 (d, J=5.7 Hz, 1H), 4.82 (p, J=7.5 Hz, 1H), 3.66 (s, 3H), 2.64-2.52 (m, 2H), 2.40-2.30 (m, 1H), 2.05 (s, 6H), 1.81 (ddd, J=16.7, 8.3, 2.7 Hz, 2H).

### Example 193: Preparation of 6-fluoro-1,8-naphthyridin-3-amine (Intermediate 79)

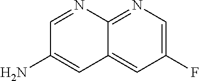 [SMILES: Nc1cnc2ncc(F)cc2c1]

### Step 1: 2-amino-5-fluoronicotinaldehyde

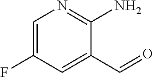

A solution of 2-Amino-3-bromo-5-fluoropyridine (1.00 g, 5.13 mmol) in THF (25 mL) was cooled to −78° C. then MeLi (3M in DME, 1.82 mL, 5.64 mmol) was added dropwise. The solution was stirred for 30 minutes before adding n-butyllithium (2.5 M in hexane, 4.52 mL, 11.3 mmol) dropwise. The mixture was stirred for 45 minutes before adding 4-formylmorpholine (1.55 mL, 15.4 mmol). The mixture was stirred for an additional 30 minutes before quenching with 0.1 M HCl. The mixture was adjusted to pH 10 and extracted four times with DCM. The combined organic layers were dried over MgSO₄, filtered and concentrated then the residue was purified by flash chromatography using 0 to 50% EtOAc in hexanes to give the title compound (425 mg, 59%) as a yellow solid. [M+H]⁺: 141.1.

### Step 2: 3-fluoro-6-nitro-1,8-naphthyridine

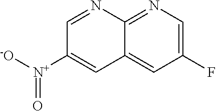

The title compound was prepared from 2-amino-5-fluoronicotinaldehyde according to 3-nitronapthiridine synthesis general procedure S and was purified by flash chromatography. [M+H]⁺: 194.1.

### Step 3: 6-fluoro-1,8-naphthyridin-3-amine

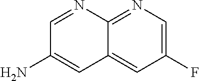 [SMILES: Nc1cnc2ncc(F)cc2c1]

The title compound was prepared from 3-fluoro-6-nitro-1,8-naphthyridine according to nitro reduction general procedure I and was purified by flash chromatography. [M+H]⁺: 164.1.

### Example 194: Preparation of Compound 86

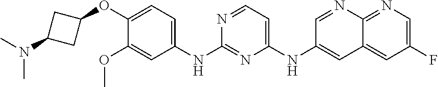 [SMILES: COc1cc(Nc2nccc(Nc3cnc4ncc(F)cc4c3)n2)ccc1O[C@H]1C[C@@H](N(C)C)C1]

Compound 86 was prepared from the corresponding intermediate according to general procedure C. MS (m/z): [M+H]⁺ 476.4. H NMR (500 MHz, DMSO) δ 10.09 (s, 1H), 9.11-9.02 (m, 3H), 8.91 (d, J=3.0 Hz, 1H), 8.13 (d, J=5.7 Hz, 1H), 7.90 (s, 1H), 7.25 (s, 1H), 7.20 (d, J=9.5 Hz, 1H), 6.80 (d, J=8.7 Hz, 1H), 6.33 (d, J=5.6 Hz, 1H), 4.34 (p, J=7.4 Hz, 1H), 3.65 (s, 3H), 2.61-2.55 (m, 2H), 2.35-2.28 (m, 1H), 2.05 (s, 6H), 1.88-1.80 (m, 2H).

### Example 195: Preparation of 6-chloro-1,7-naphthyridin-3-amine (Intermediate 80)

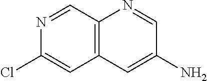 [SMILES: Nc1cnc2cnc(Cl)cc2c1]

### Step 1: tert-butyl (6-chloro-4-formylpyridin-3-yl)carbamate

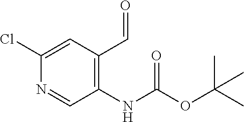 [SMILES: CC(C)(C)OC(=O)Nc1cnc(Cl)cc1C=O]

A solution of TMEDA (1.26 mL, 8.31 mmol) and (6-Chloro-pyridin-3-yl)-carbamic acid tert-butyl ester (2.00 g, 8.31 mmol) in Et2O (40.0 mL) was cooled to −78° C. and n-butyllithium (9.97 mL, 24.9 mmol) was added. The mixture was stirred at −20° C. for 1 hour then cooled to −78° C. and 4-formylmorpholine (3.34 mL, 33.2 mmol) was added. After stirring 30 minutes, pH 4 citrate buffer was added and the mixture was extracted three times with EtOAc. The combined organic layers were washed with brine, dried over MgSO₄, filtered and concentrated then the residue was purified by flash chromatography (SiO₂, EtOAc hexane) to give the title compound (920 mg, 41%) as a white solid. [M+H]⁺: 257.1.

### Step 2: 6-chloro-3-nitro-1,7-naphthyridine

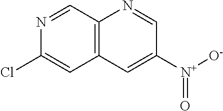 [SMILES: O=[N+]([O-])c1cnc2cnc(Cl)cc2c1]

The title compound was prepared from tert-butyl (6-chloro-4-formylpyridin-3-yl)carbamate according to 3-nitronapthiridine synthesis general procedure S and was purified by flash chromatography.

### Step 3: 6-chloro-1,7-naphthyridin-3-amine

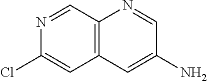 [SMILES: Nc1cnc2cnc(Cl)cc2c1]

The title compound was prepared from 6-chloro-3-nitro-1,7-naphthyridine according to nitro reduction general procedure I and the crude material was used directly in the next step. [M+H]⁺: 180.1.

### Example 196: Preparation of Compound 87

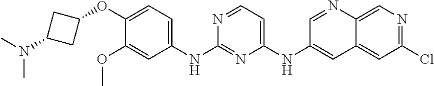 [SMILES: COc1cc(Nc2nccc(Nc3cnc4cnc(Cl)cc4c3)n2)ccc1O[C@H]1C[C@@H](N(C)C)C1]

Compound 87 was prepared from the corresponding intermediate according to general procedure C. MS (m/z): [M+H]⁺ 492.4. H NMR (500 MHz, DMSO) δ 10.28 (s, 1H), 9.14 (s, 1H), 9.11-8.94 (m, 3H), 8.16 (d, J=5.6 Hz, 1H), 7.69 (s, 1H), 7.27-7.18 (m, 2H), 6.81 (d, J=8.6 Hz, 1H), 6.36 (d, J=5.6 Hz, 1H), 4.37 (p, J=7.2 Hz, 1H), 3.68 (s, 3H), 2.63-2.53 (m, 2H), 2.36-2.27 (m, 1H), 2.05 (s, 6H), 1.85 (ddd, J=16.6, 8.3, 2.5 Hz, 2H).

### Example 197: Preparation of 8-fluoro-1,6-naphthyridin-3-amine (Intermediate 81) Synthesis of 8-fluoro-1,6-naphthyridin-3-amine

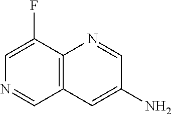 [SMILES: Nc1cnc2c(F)cncc2c1]

### Step 1: 3-bromo-5-fluoropyridin-4-amine

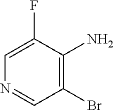

The title compound was prepared from 3-fluoropyridin-4-amine according to general procedure W and the crude material was used directly in the next step. [M+H]⁺: 191.0, 193.

### Step 2: 4-amino-5-fluoronicotinaldehyde

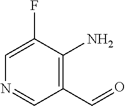

A solution of 3-bromo-5-fluoropyridin-4-amine (3.34 g, 17.5 mmol) in THF (117 mL) was cooled to −78° C. then MeLi (6.21 mL, 19.2 mmol) was added dropwise. The mixture was stirred for 20 minutes then 4-formylmorpholine (2.46 mL, 24.5 mmol) was added followed by dropwise addition of n-butyllithium (7.69 mL, 19.2 mmol). After 1 hour additional n-butyllithium (7.69 mL, 19.2 mmol) was added and the mixture was stirred 15 before additional 4-formylmorpholine (2.46 mL, 24.5 mmol) was added. After an additional 30 minutes the reaction was quenched with saturated NaHCO₃. After extracting three times with EtOAc the combined organic layers were dried over MgSO₄, filtered and concentrated then the residue was purified by Flash chromatography (SiO₂, EtOAc/hexane) to give the title compound (975 mg, 40%) as white solid. [M+H]⁺: 141.

### Step 3: 8-fluoro-3-nitro-1,6-naphthyridine

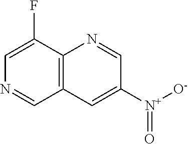 [SMILES: O=[N+]([O-])c1cnc2c(F)cncc2c1]

The title compound was prepared from 4-amino-5-fluoronicotinaldehyde according to 3-nitronapthiridine synthesis general procedure S and was purified by flash chromatography.

### Step 4: 8-fluoro-1,6-naphthyridin-3-amine

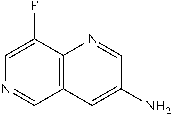 [SMILES: Nc1cnc2c(F)cncc2c1]

The title compound was prepared from 8-fluoro-3-nitro-1,6-naphthyridine according to nitro reduction general procedure I and was purified by flash chromatography. [M+H]⁺: 164.1.

### Example 198: Preparation of N2-(4-((1s,3s)-3-(dimethylamino)cyclobutoxy)-3-methoxyphenyl)-N4-(5-fluoro-1,7-naphthyridin-3-yl)pyrimidine-2,4-diamine (Compound 88)

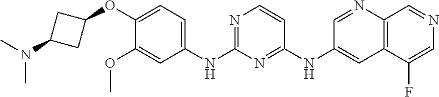 [SMILES: COc1cc(Nc2nccc(Nc3cnc4cncc(F)c4c3)n2)ccc1O[C@H]1C[C@@H](N(C)C)C1]

Cesium carbonate (212 mg, 0.645 mmol) was added to solution of 5-fluoro-1,7-naphthyridin-3-amine (35 mg (0.215 mmol), and 4-chloro-N-(4-((1s,3s)-3-(dimethylamino)cyclobutoxy)-3-methoxyphenyl)pyrimidin-2-amine (75 mg, 0.215 mmol) in DMF (2.15 mL). The mixture was heated to 100° C. and stirred for 8 hours, then was cooled and diluted with Et2O. The solid was filtered off and the filtrate was concentrated and further purified by RP flash chromatography using 20 to 100% MeOH in 10 mM ammonium bicarbonate. Appropriate fractions were combined and further purified by PREP MS using a gradient of 25 to 45% to give the title compound (5 mg, 4.9%) as a white solid. [M+H]⁺: 476.5 ¹H NMR (500 MHz, CDCl3) δ 10.17 (s, 1H), 9.23 (s, 1H), 9.19 (s, 1H), 9.15 (s, 1H), 8.97 (s, 1H), 8.56 (d, J=2.7 Hz, 1H), 8.14 (d, J=5.6 Hz, 1H), 7.26 (s, 1H), 7.20 (d, J=7.6 Hz, 1H), 6.80 (d, J=8.7 Hz, 1H), 6.34 (d, J=5.7 Hz, 1H), 4.35 (p, J=7.0 Hz, 1H), 3.67 (s, 3H), 2.62-2.56 (m, 2H), 2.38-2.31 (m, 1H), 2.08 (s, 6H), 1.86 (ddd, J=16.3, 8.6, 2.7 Hz, 2H).

### Example 199: Preparation of tert-butyl 4-((5-amino-3-methoxypyridin-2-yl)oxy)piperidine-1-carboxylate (Intermediate 82)

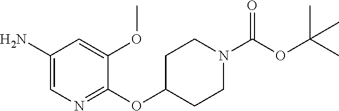

### Step 1: tert-butyl 4-((3-methoxy-5-nitropyridin-2-yl)oxy)piperidine-1-carboxylate

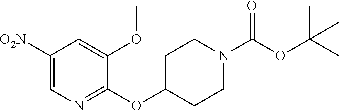

The title compound was prepared from 2-chloro-3-methoxy-5-nitropyridine and n-boc-4-piperidinol according to general procedure G and was purified by flash chromatography. [M+H]⁺: 298.2 (M-tBu+H).

### Step 2: tert-butyl 4-((5-amino-3-methoxypyridin-2-yl)oxy)piperidine-1-carboxylate

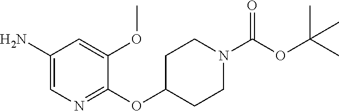

The title compound was prepared from tert-butyl 4-((3-methoxy-5-nitropyridin-2-yl)oxy)piperidine-1-carboxylate according to nitro reduction general procedure H and was purified by flash chromatography. [M+H]⁺: 324.3.

### Example 200: Preparation of N4-(6,7-difluoroquinolin-3-yl)-N2-(5-methoxy-6-(piperidin-4-yloxy)pyridin-3-yl)pyrimidine-2,4-diamine (Compound 89)

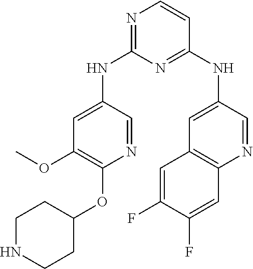 [SMILES: COc1cc(Nc2nccc(Nc3cnc4cc(F)c(F)cc4c3)n2)cnc1OC1CCNCC1]

To 5-methoxy-6-(piperidin-4-yloxy)pyridin-3-amine (75.0 mg, 232 umol) and N-(2-chloropyrimidin-4-yl)-6,7-difluoroquinolin-3-amine (67.9 mg, 232 umol) in iPrOH (2.00 mL) was charged trifluoroacetic acid (89.7 uL, 1.16 mmol). The mixture was heated to 80° C. and stirred for 1 hour then the reaction was cooled and a mixture of TFA (1.8 mL), triisopropylsilane (100 uL), and Water (100 uL) was added. The mixture was stirred at 40° C. for 1 hour then was poured into 100 mL of Et2O. The precipitated solid was collected and purified by PREP MS using a gradient of 15 to 35% CH3CN in ammonium formate to give the title compound (27.1 mg, 22%) as a white solid. ¹H NMR (500 MHz, DMSO) δ 10.09 (s, 1H), 9.19 (s, 1H), 8.96 (s, 1H), 8.38 (s, 1H), 8.10 (d, J=5.7 Hz, 1H), 8.06 (d, J=1.9 Hz, 1H), 7.93 (dd, J=11.1, 8.5 Hz, 1H), 7.70 (s, 1H), 7.59 (s, 1H), 6.35 (d, J=5.7 Hz, 1H), 5.24-5.15 (m, 1H), 3.68 (s, 3H), 3.26-3.12 (m, 2H), 3.04-2.94 (m, 2H), 2.21-1.99 (m, 2H), 1.90-1.74 (m, 2H). [M+H]⁺:480.4

### Example 201: Preparation of 2-methoxy-6-(4-methylpiperazin-1-yl)pyridin-3-amine (Intermediate 83)

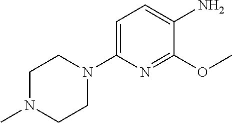

### Step 1: 1-(6-methoxy-5-nitropyridin-2-yl)-4-methylpiperazine

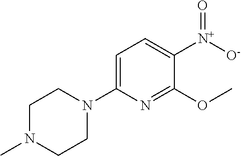 [SMILES: COc1nc(N2CCN(C)CC2)ccc1[N+](=O)[O-]]

The title compound was prepared from 6-chloro-2-methoxy-3-nitropyridine and 1-methylpiperazine according to general procedure G and the crude material was used directly in the next step. [M+H]⁺: 253.

### Step 2: 2-methoxy-6-(4-methylpiperazin-1-yl)pyridin-3-amine

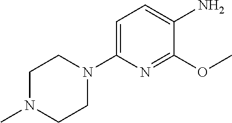

The title compound was prepared from crude 1-(6-methoxy-5-nitropyridin-2-yl)-4-methylpiperazine according to nitro reduction general procedure H and was purified by flash chromatography. [M+H]⁺: 223.3.

### Example 202: Preparation of Compound 90

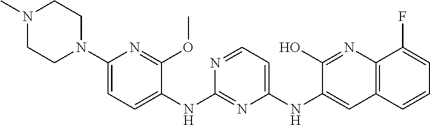 [SMILES: COc1nc(N2CCN(C)CC2)ccc1Nc1nccc(Nc2cc3cccc(F)c3nc2O)n1]

Compound 90 was prepared from the corresponding intermediate according to general procedure E. MS (m/z): [M+H]⁺ 477.4. ¹H NMR (400 MHz, DMSO) δ 8.88 (s, 1H), 8.52 (broad s, 1H), 8.29 (broad s, 1H), 8.18 (s, 1H), 7.93 (d, J=5.7 Hz, 1H), 7.50 (d, J=7.0 Hz, 1H), 7.18 (dd, J=11.0, 9.3 Hz, 1H), 7.12-6.86 (m, 2H), 6.61 (d, J=5.7 Hz, 1H), 6.35 (d, J=8.4 Hz, 1H), 3.73 (s, 3H), 3.49-3.44 (m, 4H), 2.44-2.40 (m, 4H), 2.21 (s, 3H).

### Example 203: Preparation of 4-methoxy-6-(4-methylpiperazin-1-yl)pyridin-3-amine (Intermediate 84)

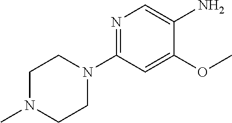

### Step 1: 1-(4-methoxy-5-nitropyridin-2-yl)-4-methylpiperazine

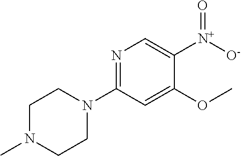 [SMILES: COc1cc(N2CCN(C)CC2)ncc1[N+](=O)[O-]]

The title compound was prepared from 2-chloro-4-methoxy-5-nitropyridine and 1-methylpiperazine according to general procedure G and the crude material was used directly in the next step. [M+H]⁺: 253.2.

### Step 2: 4-methoxy-6-(4-methylpiperazin-1-yl)pyridin-3-amine

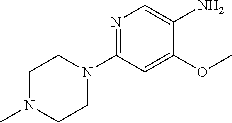

The title compound was prepared from crude 1-(4-methoxy-5-nitropyridin-2-yl)-4-methylpiperazine according to nitro reduction general procedure H and was purified by flash chromatography. [M+H]⁺: 223.3.

### Example 204: Preparation of Compound 91

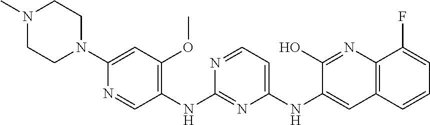 [SMILES: COc1cc(N2CCN(C)CC2)ncc1Nc1nccc(Nc2cc3cccc(F)c3nc2O)n1]

Compound 91 was prepared from the corresponding intermediate according to general procedure E. MS (m/z): [M+H]⁺ 476.5. H NMR (400 MHz, DMSO) δ 12.19 (s, 1H), 8.94 (s, 1H), 8.58 (s, 1H), 8.15 (s, 1H), 8.07 (s, 1H), 7.97 (d, J=5.7 Hz, 1H), 7.26-7.19 (m, 1H), 7.17-6.98 (m, 2H), 6.66 (d, J=5.7 Hz, 1H), 6.50 (s, 1H), 3.77 (s, 3H), 3.60-3.52 (m, 4H), 2.48-2.43 (m, 4H), 2.26 (s, 3H).

### Example 205: Preparation of 1-(6-aminoindolin-1-yl)-2-(dimethylamino)ethenone (Intermediate 85)

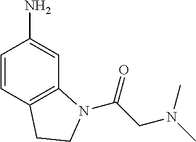 [SMILES: CN(C)CC(=O)N1CCc2ccc(N)cc21]

### Step 1: 2-(dimethylamino)-1-(6-nitroindolin-1-yl)ethanone

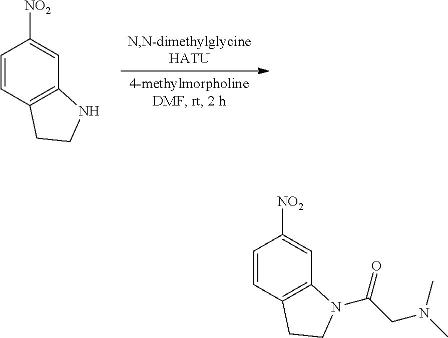 [SMILES: O=[N+]([O-])c1ccc2c(c1)NCC2 ; "4-methylmorpholineDMF, rt, 2 h" ; "N,N-dimethylglycineHATU" ; CN(C)CC(=O)N1CCc2ccc([N+](=O)[O-])cc21]

To a flask charged with 6-nitroindoline (12.9 uL, 2.95 mmol) and HATU (1.69 g, 4.43 mmol) dissolved in DMF (15 mL) was added N,N-dimethylglycine (5.16 uL, 2.95 mmol) and 4-methylmorpholine (1.21 g, 11.8 mmol). The reaction was stirred at room temperature for 2 h then quenched with NH₄Cl and extracted with EtOAc (3×). The combined organic layers were washed with water and brine before drying and concentrating. The crude reaction mixture purified by normal phase column chromatography using (0-100% EtOAc in hexanes) followed by 10% (9:1 NH₄OH/MeOH) in CHCl₃ to deliver the title compound (329 mg, 45%). MS (m/z): [M+H]⁺: 250.2

### Step 2: 1-(6-aminoindolin-1-yl)-2-(dimethylamino)ethanone

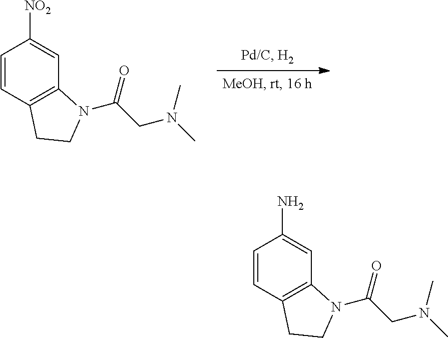 [SMILES: CN(C)CC(=O)N1CCc2ccc([N+](=O)[O-])cc21 ; "MeOH, rt, 16 h" ; "Pd/C, H2" ; CN(C)CC(=O)N1CCc2ccc(N)cc21]

The title compound was prepared from 2-(dimethylamino)-1-(6-nitroindolin-1-yl)ethanone according to general procedure H. MS (m/z): [M+H]⁺: 220.1 Example 206: Preparation of Compound 92

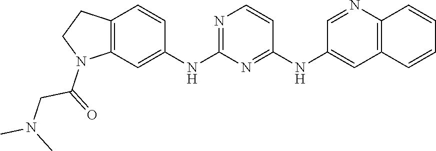 [SMILES: CN(C)CC(=O)N1CCc2ccc(Nc3nccc(Nc4cnc5ccccc5c4)n3)cc21]

Compound 92 was prepared from the corresponding intermediate according to general procedure E. MS (m/z): [M+H]⁺ 440.5. H NMR (500 MHz, DMSO) δ 9.87 (s, 1H), 9.28 (s, 1H), 9.10 (s, 1H), 8.90 (d, J=2.1 Hz, 1H), 8.39 (s, 1H), 8.10 (d, J=5.7 Hz, 1H), 7.91 (dd, J=6.0, 3.5 Hz, 1H), 7.68 (s, 1H), 7.61-7.53 (m, 2H), 7.45 (s, 1H), 7.16 (d, J=8.1 Hz, 1H), 6.34 (d, J=5.7 Hz, 1H), 4.18 (t, J=8.3 Hz, 2H), 3.13 (m, 4H), 2.11 (s, 6H).

### Example 207: Preparation of 1-(6-aminoindolin-1-yl)-2-(pyrrolidin-1-yl)ethanone (Intermediate 86)

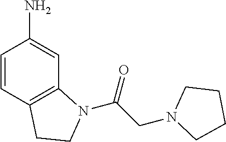 [SMILES: Nc1ccc2c(c1)N(C(=O)CN1CCCC1)CC2]

### Step 1: 2-chloro-1-(6-nitroindolin-1-yl)ethanone

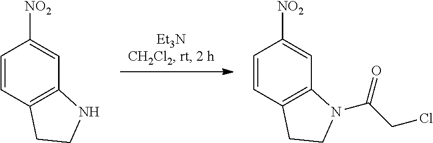 [SMILES: O=[N+]([O-])c1ccc2c(c1)NCC2 ; "Et3NCH2Cl2, rt, 2 h" ; O=C(CCl)N1CCc2ccc([N+](=O)[O-])cc21]

The title compound was prepared from 6-nitroindoline according to general procedure K. MS (m/z): [M+H]⁺: 240.6

### Step 2: 1-(6-nitroindolin-1-yl)-2-(pyrrolidin-1-yl)ethanone

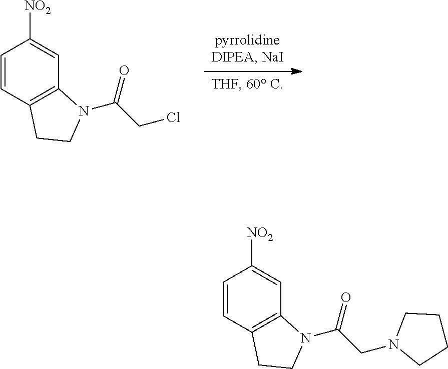 [SMILES: O=C(CCl)N1CCc2ccc([N+](=O)[O-])cc21 ; "THF, 60° C." ; "pyrrolidineDIPEA, NaI" ; O=C(CN1CCCC1)N1CCc2ccc([N+](=O)[O-])cc21]

2-Chloro-1-(6-nitroindolin-1-yl)ethanone (200 mg, 831 umol) and sodium iodide (200 mg, 1.33 mmol) were dissolved in THF (5.00 mL) and stirred. Pyrrolidine (34.5 uL, 997 umol) and N,N-diisopropylethylamine (436 uL, 2.49 mmol) were then added and the reaction was stirred overnight at 60 C. The crude reaction was purified by reverse phase chromatography on a C18 column, eluting with a solvent gradient of 15-40% MeCN/H2O (0.1% of 10 mM ammonium formate buffer). The clean fractions were combined and lyophilized to afford the title product (55.0 mg, 24%). MS (m/z): [M+H]⁺: 276.3

### Step 3: 1-(6-aminoindolin-1-yl)-2-(pyrrolidin-1-yl)ethanone

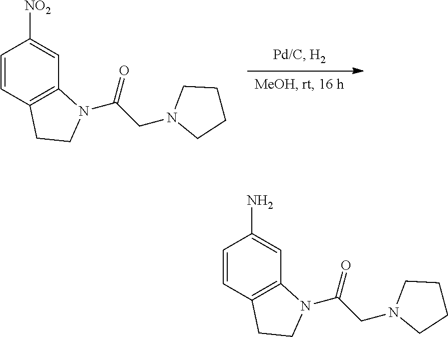 [SMILES: O=C(CN1CCCC1)N1CCc2ccc([N+](=O)[O-])cc21 ; "MeOH, rt, 16 h" ; "Pd/C, H2" ; Nc1ccc2c(c1)N(C(=O)CN1CCCC1)CC2]

The title compound was prepared from 1-(6-nitroindolin-1-yl)-2-(pyrrolidin-1-yl)ethanone according to general procedure H. MS (m/z): [M+H]⁺: 246.3 Example 208: Preparation of Compound 93

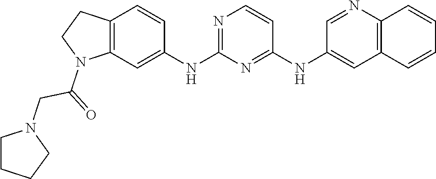 [SMILES: O=C(CN1CCCC1)N1CCc2ccc(Nc3nccc(Nc4cnc5ccccc5c4)n3)cc21]

Compound 93 was prepared from the corresponding intermediate according to general procedure E. MS (m/z): [M+H]⁺ 466.4. ¹H NMR (500 MHz, MeOD) δ 8.93 (d, J=2.4 Hz, 1H), 8.75 (d, J=2.5 Hz, 1H), 8.42 (d, J=1.5 Hz, 2H), 7.93 (d, J=5.8 Hz, 1H), 7.82 (d, J=8.1 Hz, 1H), 7.54-7.44 (m, 2H), 7.42 (d, J=7.7 Hz, 1H), 7.17-7.08 (m, 2H), 6.21 (d, J=5.8 Hz, 1H), 4.01 (t, J=8.3 Hz, 2H), 3.82 (s, 2H), 3.22 (s, 2H), 2.82 (s, 4H), 1.80 (s, 4H).

### Example 209: Preparation of 3-methoxy-4-(4-methylpiperazin-1-yl)aniline (Intermediate 87)

### Step 1: 1-(2-methoxy-4-nitrophenyl)-4-methylpiperazine

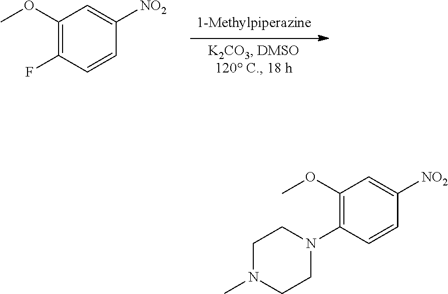 [SMILES: COc1cc([N+](=O)[O-])ccc1F ; "1-Methylpiperazine" ; "K2CO3, DMSO120° C., 18 h" ; COc1cc([N+](=O)[O-])ccc1N1CCN(C)CC1]

The title compound was prepared from 1-fluoro-2-methoxy-4-nitrobenzene and 1-methylpiperazine according to general procedure G. MS (m/z): [M+H]⁺: 252.3

### Step 2: 3-methoxy-4-(4-methylpiperazin-1-yl)aniline

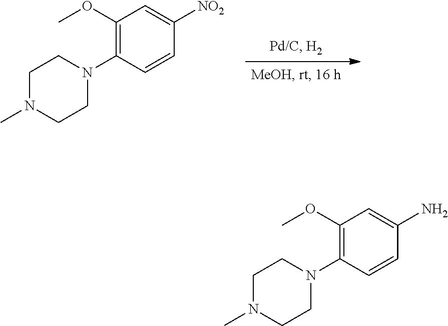 [SMILES: COc1cc([N+](=O)[O-])ccc1N1CCN(C)CC1 ; "MeOH, rt, 16 h" ; "Pd/C, H2" ; COc1cc(N)ccc1N1CCN(C)CC1]

The title compound was prepared from 1-(2-methoxy-4-nitrophenyl)-4-methylpiperazine according to general procedure H. MS (m/z): [M+H]⁺: 222.2 Example 210: Preparation of Compound 94

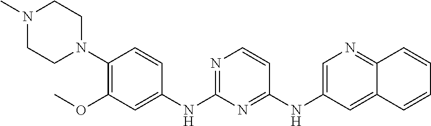 [SMILES: COc1cc(Nc2nccc(Nc3cnc4ccccc4c3)n2)ccc1N1CCN(C)CC1]

Compound 94 was prepared from the corresponding intermediate according to general procedure E. MS (m/z): [M+H]⁺ 442.5. ¹H NMR (500 MHz, DMSO) δ 9.83 (s, 1H), 9.10 (s, 1H), 9.01 (s, 1H), 8.92 (d, J=2.2 Hz, 1H), 8.09 (d, J=5.6 Hz, 1H), 7.93 (d, J=8.5 Hz, 1H), 7.73 (s, 1H), 7.62-7.56 (m, 1H), 7.52 (t, J=7.1 Hz, 1H), 7.25 (d, J=12.7 Hz, 2H), 6.83 (d, J=8.4 Hz, 1H), 6.31 (d, J=5.7 Hz, 1H), 3.66 (s, 3H), 2.97 (s, 4H), 2.48 (s, 4H), 2.24 (s, 3H).

### Example 211: Preparation of 1-(4-amino-2-methoxyphenyl)-N,N-dimethylpiperidin-4-amine (Intermediate 88)

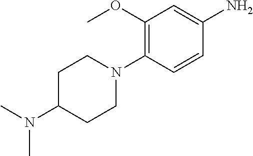 [SMILES: COc1cc(N)ccc1N1CCC(N(C)C)CC1]

### Step 1: 1-(2-methoxy-4-nitrophenyl)-N,N-dimethylpiperidin-4-amine

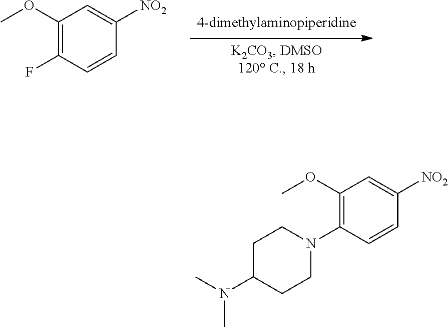 [SMILES: COc1cc([N+](=O)[O-])ccc1F ; "4-dimethylaminopiperidine" ; "K2CO3, DMSO120° C., 18 h" ; COc1cc([N+](=O)[O-])ccc1N1CCC(N(C)C)CC1]

The title compound was prepared from 1-Fluoro-2-methoxy-4-nitrobenzene and 4-dimethylaminopiperidine according to general procedure G. MS (m/z): [M+H]⁺: 280.3

### Step 2: 1-(4-amino-2-methoxyphenyl)-N,N-dimethylpiperidin-4-amine

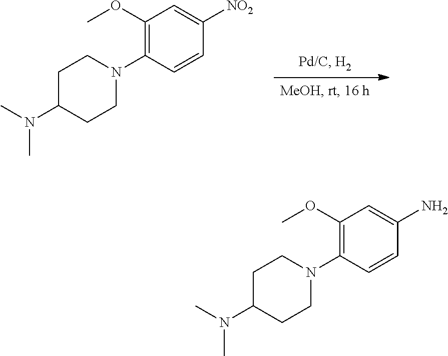 [SMILES: COc1cc([N+](=O)[O-])ccc1N1CCC(N(C)C)CC1 ; "MeOH, rt, 16 h" ; "Pd/C, H2" ; COc1cc(N)ccc1N1CCC(N(C)C)CC1]

The title compound was prepared from 1-(2-methoxy-4-nitrophenyl)-N,N-dimethylpiperidin-4-amine according to general procedure H. MS (m/z): [M+H]⁺: 250.3 Example 212: Preparation of Compound 95

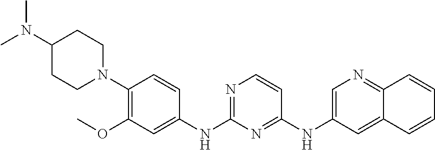 [SMILES: COc1cc(Nc2nccc(Nc3cnc4ccccc4c3)n2)ccc1N1CCC(N(C)C)CC1]

Compound 95 was prepared from the corresponding intermediate according to general procedure E. MS (m/z): [M+H]⁺ 468.4. ¹H NMR (500 MHz, DMSO) δ 9.83 (s, 1H), 9.09 (s, 1H), 9.01 (s, 1H), 8.92 (d, J=2.0 Hz, 1H), 8.09 (d, J=5.7 Hz, 1H), 7.93 (d, J=8.4 Hz, 1H), 7.73 (s, 1H), 7.63-7.57 (m, 1H), 7.53 (t, J=7.1 Hz, 1H), 7.24 (d, J=8.7 Hz, 2H), 6.84 (d, J=8.3 Hz, 1H), 6.30 (d, J=5.7 Hz, 1H), 3.66 (s, 3H), 3.38 (d, J=11.6 Hz, 2H), 2.55 (d, J=11.8 Hz, 2H), 2.23 (s, 6H), 2.21-2.16 (m, 1H), 1.84 (d, J=10.6 Hz, 2H), 1.59-1.51 (m, 2H).

### Example 213: Preparation of 8-fluoroquinolin-3-amine (Intermediate 89)

### Step 1: Preparation of 8-fluoroquinolin-3-amine

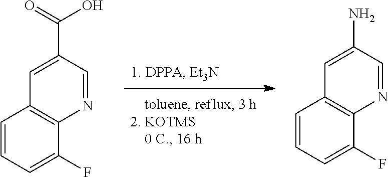 [SMILES: O=C(O)c1cnc2c(F)cccc2c1 ; "1. DPPA, Et3N" ; Nc1cnc2c(F)cccc2c1]

The title compound was prepared from 8-Fluoroquinoline-3-carboxylic acid according to general procedure O. MS (m/z): [M+H]⁺: 163.2 Example 214: Preparation of Compound 96

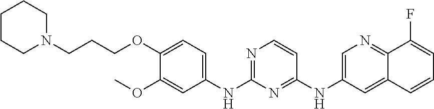 [SMILES: COc1cc(Nc2nccc(Nc3cnc4c(F)cccc4c3)n2)ccc1OCCCN1CCCCC1]

Compound 96 was prepared from the corresponding intermediate according to general procedure C. MS (m/z): [M+H]⁺ 503.4. H NMR (500 MHz, DMSO) δ 9.96 (s, 1H), 9.12 (s, 1H), 9.05 (s, 1H), 8.99 (s, 1H), 8.12 (d, J=5.7 Hz, 1H), 7.56 (s, 1H), 7.52 (td, J=7.8, 4.9 Hz, 1H), 7.39 (dd, J=11.0, 8.8 Hz, 1H), 7.30 (s, 1H), 7.23 (d, J=7.9 Hz, 1H), 6.90 (d, J=8.7 Hz, 1H), 6.33 (d, J=5.7 Hz, 1H), 3.98 (t, J=6.4 Hz, 2H), 3.66 (s, 3H), 2.48-2.33 (m, 6H), 1.90 (s, 2H), 1.53 (s, 4H), 1.41 (s, 2H).

### Example 215: Preparation of 7-fluoroquinolin-3-amine (Intermediate 90)

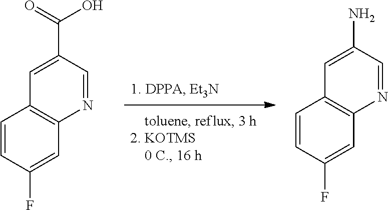 [SMILES: O=C(O)c1cnc2cc(F)ccc2c1 ; "1. DPPA, Et3N" ; Nc1cnc2cc(F)ccc2c1]

The title compound was prepared from 7-Fluoroquinoline-3-carboxylic acid according to general procedure O. MS (m/z): [M+H]⁺: 163.1 Example 216: Preparation of Compound 97

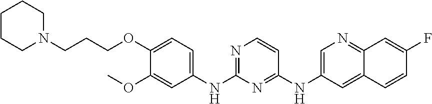 [SMILES: COc1cc(Nc2nccc(Nc3cnc4cc(F)ccc4c3)n2)ccc1OCCCN1CCCCC1]

Compound 97 was prepared from the corresponding intermediate according to general procedure C. MS (m/z): [M+H]⁺ 503.4. ¹H NMR (500 MHz, DMSO) δ 9.84 (s, 1H), 9.08 (s, 1H), 9.01 (s, 1H), 8.98 (s, 1H), 8.09 (d, J=5.7 Hz, 1H), 7.84 (s, 1H), 7.68 (dd, J=10.4, 2.6 Hz, 1H), 7.52 (td, J=8.7, 2.6 Hz, 1H), 7.31 (s, 1H), 7.24 (d, J=8.2 Hz, 1H), 6.88 (d, J=8.7 Hz, 1H), 6.30 (d, J=5.7 Hz, 1H), 3.98 (t, J=6.4 Hz, 2H), 3.65 (s, 3H), 2.41 (s, 4H), 1.90 (s, 2H), 1.53 (s, 4H), 1.41 (s, 2H). *CH₂ peak missing due to overlap with solvent.

### Example 217: Preparation of 7-methylquinolin-3-amine (Intermediate 91)

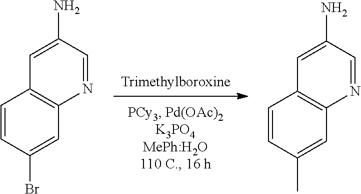

The title compound was prepared from 7-bromoquinolin-3-amine according to general procedure P. MS (m/z): [M+H]⁺: 159.2 Example 218: Preparation of Compound 98

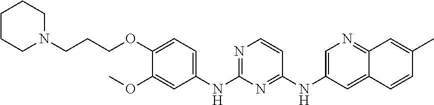 [SMILES: COc1cc(Nc2nccc(Nc3cnc4cc(C)ccc4c3)n2)ccc1OCCCN1CCCCC1]

Compound 98 was prepared from the corresponding intermediate according to general procedure C. MS (m/z): [M+H]⁺ 499.6. H NMR (500 MHz, DMSO) δ 9.78 (s, 1H), 9.07 (s, 1H), 8.90 (s, 2H), 8.07 (d, J=5.7 Hz, 1H), 7.72 (s, 1H), 7.66 (s, 1H), 7.39 (dd, J=8.4, 1.4 Hz, 1H), 7.31 (s, 1H), 7.24 (d, J=8.1 Hz, 1H), 6.88 (d, J=8.7 Hz, 1H), 6.29 (d, J=5.7 Hz, 1H), 3.98 (t, J=6.3 Hz, 2H), 3.64 (s, 3H), 2.58 (s, 4H), 1.99-1.88 (m, 2H), 1.56 (d, J=5.2 Hz, 4H), 1.43 (s, 2H).*one CH₃ peak missing due to overlap with solvent.

### Example 219: Preparation of 8-methylquinolin-3-amine (Intermediate 92)

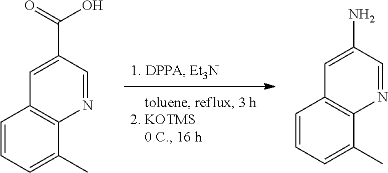

The title compound was prepared from 8-methylquinoline-3-carboxylic acid according to general procedure O. MS (m/z): [M+H]⁺: 159.2 Example 220: Preparation of Compound 99

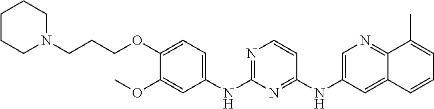 [SMILES: COc1cc(Nc2nccc(Nc3cnc4c(C)cccc4c3)n2)ccc1OCCCN1CCCCC1]

Compound 99 was prepared from the corresponding intermediate according to general procedure C. MS (m/z): [M+H]⁺ 499.5. H NMR (500 MHz, DMSO) δ 9.82 (s, 1H), 9.09 (s, 1H), 9.00 (s, 1H), 8.90 (s, 1H), 8.09 (d, J=5.7 Hz, 1H), 7.59 (s, 1H), 7.51-7.38 (m, 2H), 7.31 (s, 1H), 7.25 (d, J=8.4 Hz, 1H), 6.88 (d, J=8.7 Hz, 1H), 6.31 (d, J=5.7 Hz, 1H), 3.98 (t, J=6.4 Hz, 2H), 3.65 (s, 3H), 2.70 (s, 3H), 2.66-2.46 (m, 6H), 1.92 (br s, 2H), 1.55 (br s, 4H), 1.42 (br s, 2H).

### Example 221: Preparation of 2-methyl-2,3-dihydro-[1,4]dioxino[2,3-b]pyridin-7-amine (Intermediate 93)

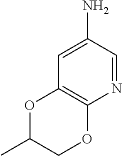

### Step 1: 2-methyl-7-nitro-2,3-dihydro-[1,4]dioxino[2,3-b]pyridine

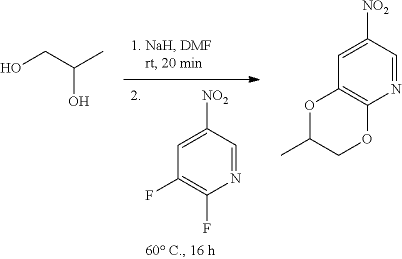

To 1,2-propanediol (112 uL, 1.53 mmol) dissolved in DMF (3 mL was added sodium hydride (60% in dispersion in mineral oil, 1.53 mmol) and stirred for 20 min. 2,3-difluoro-5-nitropyridine (100 mg, 612 umol) dissolved in DMF was then added and stirred at 60° C. for 16 h. The reaction was quenched with NH₄Cl, extracted with EtOAc (3×) and washed with H2O/brine. The collected organic layers were dried and concentrated onto silica for purification by flash column chromatography on silica gel column, eluting with a gradient of 0-25% EtOAc/hexanes to afford the title product (31.0 mg, 26%). MS (m/z): [M+H]⁺: 197.1

### Step 2: 2-methyl-2,3-dihydro-[1,4]dioxino[2,3-b]pyridin-7-amine

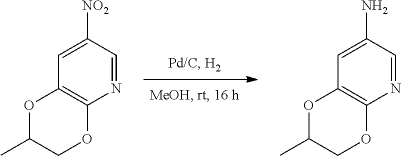

The title compound was prepared from 2-methyl-7-nitro-2,3-dihydro-[1,4]dioxino[2,3-b]pyridine according to general procedure H. MS (m/z): [M+H]⁺: 167.1 Example 222: Preparation of Compound 100

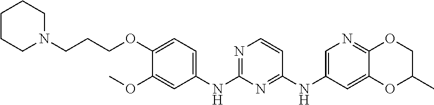 [SMILES: COc1cc(Nc2nccc(Nc3cnc4c(c3)OC(C)CO4)n2)ccc1OCCCN1CCCCC1]

Compound 100 was prepared from the corresponding intermediate according to general procedure C. MS (m/z): [M+H]⁺ 507.4. ¹H NMR (500 MHz, DMSO) δ 9.28 (s, 1H), 8.98 (s, 1H), 8.02 (s, 1H), 7.98 (d, J=5.7 Hz, 1H), 7.79 (d, J=1.9 Hz, 1H), 7.32 (s, 1H), 7.24 (d, J=8.3 Hz, 1H), 6.83 (d, J=8.7 Hz, 1H), 6.13 (d, J=5.7 Hz, 1H), 4.46-4.38 (m, 1H), 4.30 (dd, J=11.4, 2.3 Hz, 1H), 3.93 (t, J=6.4 Hz, 2H), 3.84 (dd, J=11.4, 7.9 Hz, 1H), 3.68 (s, 3H), 2.42 (br s, 4H), 1.87 (br s, 2H), 1.53 (br s, 4H), 1.41 (br s, 2H), 1.34 (d, J=6.4 Hz, 3H).

### Example 223: Preparation of 3-methyl-2,3-dihydro-[1,4]dioxino[2,3-b]pyridin-7-amine (Intermediate 94)

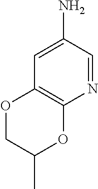

### Step 1: 7-bromo-3-methyl-2,3-dihydro-[1,4]dioxino[2,3-b]pyridine

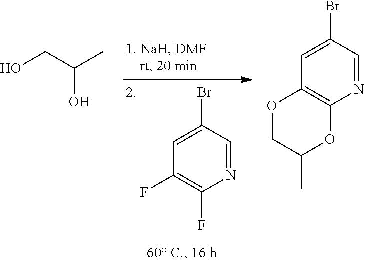

To 1,2-propanediol (855 uL, 11.6 mmol) dissolved in DMF (23 mL) was added sodium hydride (60% in dispersion in mineral oil) (11.6 mmol) and the reaction stirred for 20 min at room temperature. 5-Bromo-2-chloro-3-fluoropyridine (1.00 g, 4.66 mmol) was then added and the reaction was heated to 100° C. and stirred overnight. The reaction was quenched with NH4Cl and extracted with EtOAc (3×). The crude reaction was purified by flash column chromatography on silica gel column, eluting with a solvent gradient of 0-30% EtOAc/hexanes in order to deliver the title compound (93.0 mg, 6.9%). MS (m/z): [M+H]⁺: 230.0, 232.0

### Step 2: 3-methyl-2,3-dihydro-[1,4]dioxino[2,3-b]pyridin-7-amine

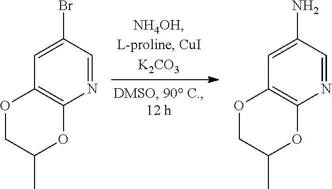

Potassium carbonate (67.0 mg, 485 umol), L-proline (14.9 mg, 128 umol), copper (I) iodide (12.4 mg, 64.7 umol), and 7-bromo-3-methyl-2,3-dihydro-[1,4]dioxino[2,3-b]pyridine (93.0 mg, 323 umol) were combined as solids in a reaction vessel, followed by purging with nitrogen. DMSO (1.00 mL) was added and stirred for 5 minutes before added ammonium hydroxide (30% solution) (20 uL, 485 umol). The mixture was heated to 90° C. and stirring was continued overnight. The reaction was quenched with water, extracted with diethyl ether, and purified by reverse phase chromatography on a C18 column, eluting with a solvent gradient of 5-25% MeCN/H2O (0.1% of 10 mM ammonium formate buffer). The clean fractions were combined and lyophilized to afford the title product (13.4 mg, 25%). MS (m/z): [M+H]⁺: 167.1

### Example 224: Preparation of Compound 101

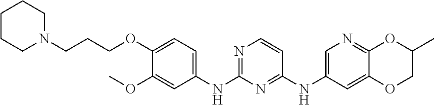 [SMILES: COc1cc(Nc2nccc(Nc3cnc4c(c3)OCC(C)O4)n2)ccc1OCCCN1CCCCC1]

Compound 101 was prepared from the corresponding intermediate according to general procedure C. MS (m/z): [M+H]⁺ 507.4. ¹H NMR (500 MHz, DMSO) δ 9.32 (s, 1H), 9.01 (s, 1H), 8.04-7.97 (m, 2H), 7.84 (d, J=2.3 Hz, 1H), 7.32 (s, 1H), 7.26 (d, J=8.7 Hz, 1H), 6.86 (d, J=8.7 Hz, 1H), 6.16 (d, J=5.7 Hz, 1H), 4.45 (dd, J=11.6, 2.3 Hz, 1H), 4.35 (ddd, J=10.6, 6.4, 3.2 Hz, 1H), 4.00 (dd, J=11.6, 8.0 Hz, 1H), 3.96 (t, J=6.5 Hz, 2H), 3.71 (s, 3H), 2.44-2.39 (m, 2H), 2.36 (br s, 4H), 1.90-1.83 (m, 2H), 1.52 (dd, J=11.1, 5.5 Hz, 4H), 1.42 (d, J=5.2 Hz, 2H), 1.35 (d, J=6.4 Hz, 3H).

### Example 225: Preparation of 6-cyclopropylquinolin-3-amine (Intermediate 95)

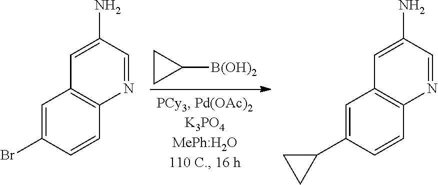

The title compound was prepared from 6-bromoquinolin-3-amine according to general procedure P. MS (m/z): [M+H]⁺: 185.2 Example 226: Preparation of Compound 102

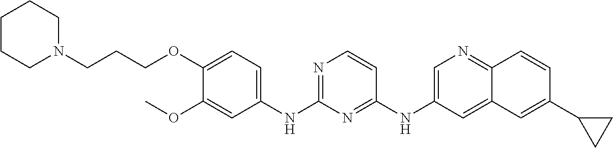 [SMILES: COc1cc(Nc2nccc(Nc3cnc4ccc(C5CC5)cc4c3)n2)ccc1OCCCN1CCCCC1]

Compound 102 was prepared from the corresponding intermediate according to general procedure C. MS (m/z): [M+H]⁺ 525.5. H NMR (500 MHz, DMSO) δ 9.70 (s, 1H), 9.00 (s, 1H), 8.80 (s, 1H), 8.74 (d, J=2.4 Hz, 1H), 8.00 (d, J=5.7 Hz, 1H), 7.73 (d, J=8.6 Hz, 1H), 7.32 (s, 1H), 7.23 (dd, J=8.7, 2.0 Hz, 2H), 7.14 (d, J=8.2 Hz, 1H), 6.81 (d, J=8.7 Hz, 1H), 6.22 (d, J=5.7 Hz, 1H), 3.90 (t, J=6.4 Hz, 2H), 3.57 (s, 3H), 2.38 (s, 2H), 2.31 (d, J=11.4 Hz, 4H), 2.02 (ddd, J=13.4, 8.4, 5.0 Hz, 1H), 1.83-1.76 (m, 2H), 1.43 (dt, J=11.0, 5.5 Hz, 4H), 1.32 (d, J=5.0 Hz, 2H), 1.01-0.94 (m, 2H), 0.81-0.73 (m, 2H).

### Example 227: Preparation of Compound 103

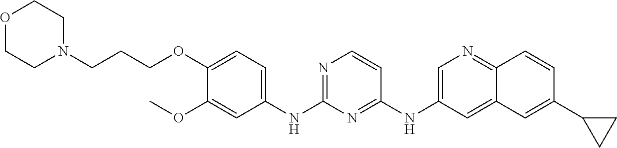 [SMILES: COc1cc(Nc2nccc(Nc3cnc4ccc(C5CC5)cc4c3)n2)ccc1OCCCN1CCOCC1]

Compound 103 was prepared from the corresponding intermediate according to general procedure C. MS (m/z): [M+H]⁺ 527. ¹H NMR (500 MHz, DMSO) δ 9.78 (s, 1H), 9.07 (s, 1H), 8.87 (s, 1H), 8.81 (d, J=2.3 Hz, 1H), 8.08 (d, J=5.7 Hz, 1H), 7.80 (d, J=8.6 Hz, 1H), 7.39 (s, 1H), 7.31 (dd, J=8.7, 2.0 Hz, 1H), 7.21 (d, J=8.6 Hz, 1H), 6.89 (d, J=8.7 Hz, 1H), 6.30 (d, J=5.7 Hz, 1H), 3.98 (t, J=6.5 Hz, 2H), 3.64 (s, 3H), 3.62-3.49 (m, 4H), 2.47-2.40 (m, 2H), 2.37 (d, J=1.8 Hz, 4H), 2.09 (m, 1H), 1.94-1.82 (m, 2H), 1.05 (m, 2H), 0.83 (dt, J=6.5, 4.4 Hz, 2H).

### Example 228: Preparation of 6-ethylquinolin-3-amine (Intermediate 96)

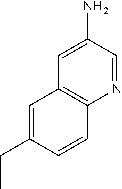

### Step 1: Preparation of 6-vinylquinolin-3-amine

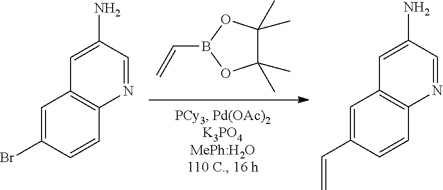

The title compound was prepared from 6-bromoquinolin-3-amine according to general procedure P. MS (m/z): [M+H]⁺: 171.1

### Step 2: Preparation of 6-ethylquinolin-3-amine

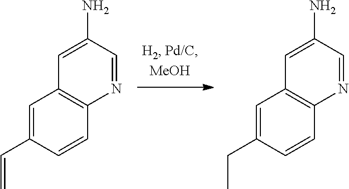

The title compound was prepared from 6-Vinylquinolin-3-amine according to general procedure H with MeOH. MS (m/z): [M+H]⁺: 173.2 Example 229: Preparation of Compound 104

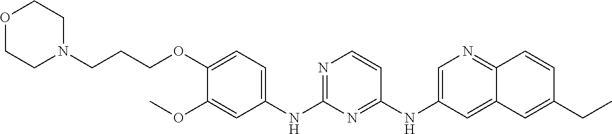 [SMILES: CCc1ccc2ncc(Nc3ccnc(Nc4ccc(OCCCN5CCOCC5)c(OC)c4)n3)cc2c1]

Compound 104 was prepared from the corresponding intermediate according to general procedure C. MS (m/z): [M+H]⁺ 515.5. ¹H NMR (500 MHz, DMSO) δ 9.88 (s, 1H), 9.16 (s, 1H), 9.02 (s, 1H), 8.92 (d, J=2.3 Hz, 1H), 8.17 (d, J=5.7 Hz, 1H), 7.92 (d, J=8.5 Hz, 1H), 7.59 (s, 1H), 7.55 (dd, J=8.5, 1.9 Hz, 1H), 7.39 (s, 1H), 7.31 (d, J=8.2 Hz, 1H), 6.98 (d, J=8.7 Hz, 1H), 6.39 (d, J=5.7 Hz, 1H), 4.07 (t, J=6.4 Hz, 2H), 3.72 (s, 3H), 3.70-3.64 (m, 4H), 2.88 (q, J=7.4 Hz, 2H), 2.56-2.50 (m, 2H), 2.46 (s, 4H), 2.02-1.92 (m, 2H), 1.39 (t, J=7.6 Hz, 3H).

### Example 230: Preparation of (R)-2-methyl-2,3-dihydro-[1,4]dioxino[2,3-b]pyridin-7-amine (Intermediate

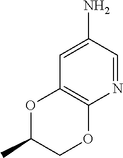

### Step 1: (R)-2-methyl-7-nitro-2,3-dihydro-[1,4]dioxino[2,3-b]pyridine

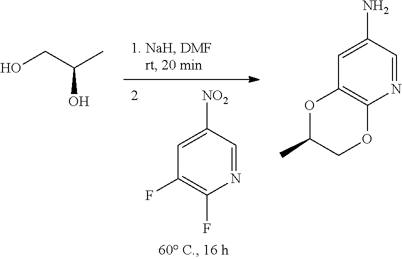

To (R)-(−)-1,2-propanediol (112 uL, 1.53 mmol) dissolved in DMF (3 mL) was added sodium hydride (60% in dispersion in mineral oil) (1.53 mmol) and stirred for 20 min. 2,3-difluoro-5-nitropyridine (100 mg, 612 umol) dissolved in DMF was then added and stirring was continued at 60° C. for 16 h. The reaction was quenched with NH₄Cl, extracted with EtOAc (3×) and washed with H2O/brine. The collected organic layers were dried and concentrated onto silica for purification by flash column chromatography on silica gel column, eluting with a solvent gradient of 0-25% EtOAc/hexanes to afford the title product (20 mg, 17%). MS (m/z): [M+H]⁺: 197.1

### Step 2: (R)-2-methyl-2,3-dihydro-[1,4]dioxino[2,3-b]pyridin-7-amine

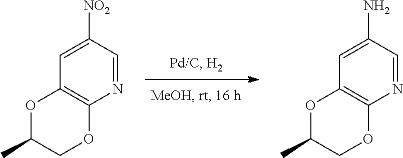

The title compound was prepared from (R)-2-methyl-7-nitro-2,3-dihydro-[1,4]dioxino[2,3-b]pyridine according to general procedure H. MS (m/z): [M+H]⁺: 166.9 Example 231: Preparation of Compound 105

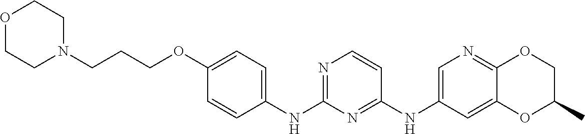 [SMILES: C[C@@H]1COc2ncc(Nc3ccnc(Nc4ccc(OCCCN5CCOCC5)cc4)n3)cc2O1]

Compound 105 was prepared from the corresponding intermediate according to general procedure C. MS (m/z): [M+H]⁺ 479.4. H NMR (500 MHz, DMSO) δ 9.38 (s, 1H), 9.10 (s, 1H), 8.06 (t, J=7.3 Hz, 2H), 7.90 (d, J=2.0 Hz, 1H), 7.65 (d, J=8.9 Hz, 2H), 6.95-6.87 (m, 2H), 6.20 (d, J=5.7 Hz, 1H), 4.58-4.48 (m, 1H), 4.40 (dd, J=11.4, 2.3 Hz, 1H), 4.05 (t, J=6.4 Hz, 2H), 3.94 (dd, J=11.4, 7.9 Hz, 1H), 3.66 (t, J=4.6 Hz, 4H), 2.51 (t, J=7.2 Hz, 2H), 2.46 (s, 4H), 2.02-1.90 (m, 2H), 1.42 (d, J=6.4 Hz, 3H).

### Example 232: Preparation of (S)-2-methyl-2,3-dihydro-[1,4]dioxino[2,3-b]pyridin-7-amine (Intermediate 9-8)

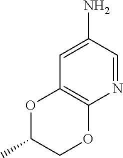

### Step 1: (S)-2-methyl-7-nitro-2,3-dihydro-[1,4]dioxino[2,3-b]pyridine

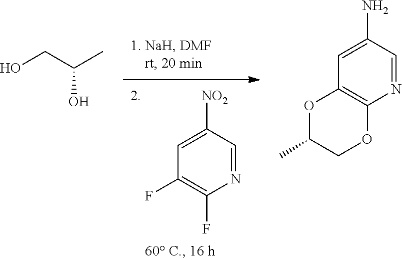

To (S)-(−)-1,2-propanediol (112 uL, 1.53 mmol) dissolved in DMF (3 mL) was added sodium hydride (60% in dispersion in mineral oil) (1.53 mmol) and stirred for 20 min. 2,3-difluoro-5-nitropyridine (100 mg, 612 umol) dissolved in DMF was then added to the primary reaction mixture stirring was continued at 60° C. for 16 h. The reaction was quenched with NH₄Cl, extracted with EtOAc (3×) and washed with H2O/brine. The collected organic layers were dried and concentrated onto silica for purification by flash column chromatography on silica gel column, eluting with a solvent gradient of 0-25% EtOAc/hexanes to afford the title product (20 mg, 17%). MS (m/z): [M+H]⁺: 197.1

### Step 2: (S)-2-methyl-2,3-dihydro-[1,4]dioxino[2,3-b]pyridin-7-amine

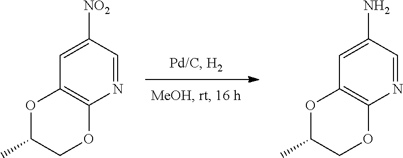

The title compound was prepared in an 79% yield from (S)-2-methyl-7-nitro-2,3-dihydro-[1,4]dioxino[2,3-b]pyridine according to general procedure H. MS (m/z): [M+H]⁺: 166.9

### Example 233: Preparation of Compound 106

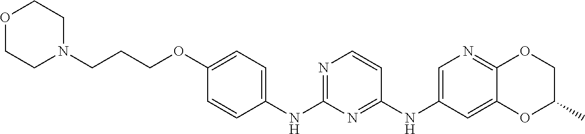 [SMILES: C[C@H]1COc2ncc(Nc3ccnc(Nc4ccc(OCCCN5CCOCC5)cc4)n3)cc2O1]

Compound 106 was prepared from the corresponding intermediate according to general procedure C. MS (m/z): [M+H]⁺ 479.3. H NMR (500 MHz, DMSO) δ 9.29 (s, 1H), 9.02 (s, 1H), 7.96 (d, J=5.7 Hz, 2H), 7.81 (d, J=2.0 Hz, 1H), 7.56 (d, J=8.9 Hz, 2H), 6.86-6.79 (m, 2H), 6.11 (d, J=5.7 Hz, 1H), 4.46-4.39 (m, 1H), 4.32 (dd, J=11.4, 2.3 Hz, 1H), 3.96 (t, J=6.4 Hz, 2H), 3.86 (dd, J=11.4, 7.9 Hz, 1H), 3.58 (t, J=4.6 Hz, 4H), 2.42 (t, J=7.2 Hz, 2H), 2.37 (s, 4H), 1.87 (dd, J=13.8, 6.7 Hz, 2H), 1.34 (d, J=6.4 Hz, 3H).

### Example 234: Preparation of 7-cyclopropylquinolin-3-amine (Intermediate 99)

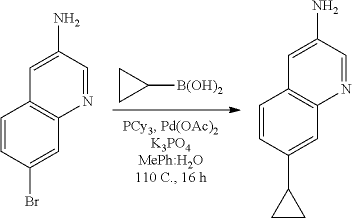

The title compound was prepared from 7-bromoquinolin-3-amine according to general procedure P. MS (m/z): [M+H]⁺: 184.9 Example 235: Preparation of Compound 107

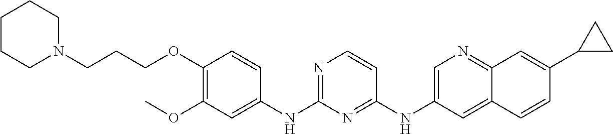 [SMILES: COc1cc(Nc2nccc(Nc3cnc4cc(C5CC5)ccc4c3)n2)ccc1OCCCN1CCCCC1]

Compound 107 was prepared from the corresponding intermediate according to general procedure C. MS (m/z): [M+H]⁺ 525.6. H NMR (500 MHz, MeOD) δ 8.76 (d, J=2.4 Hz, 1H), 8.63 (s, 1H), 7.81 (d, J=6.4 Hz, 1H), 7.53 (s, 1H), 7.50 (d, J=8.5 Hz, 1H), 7.22 (dd, J=8.5, 1.7 Hz, 1H), 7.08 (d, J=2.3 Hz, 1H), 6.93 (dd, J=8.5, 2.4 Hz, 1H), 6.88 (d, J=8.6 Hz, 1H), 6.29 (d, J=6.6 Hz, 1H), 4.07 (t, J=5.6 Hz, 2H), 3.55 (s, 3H), 3.33-3.26 (m, 2H), 2.89 (s, 4H), 2.19 (dt, J=12.4, 6.0 Hz, 2H), 2.02 (td, J=8.4, 4.2 Hz, 1H), 1.90 (t, J=14.1 Hz, 2H), 1.71 (s, 4H), 1.02 (ddd, J=8.3, 6.4, 4.5 Hz, 2H), 0.80-0.70 (m, 2H).

### Example 236: Preparation of 7-ethylquinolin-3-amine (Intermediate 100)

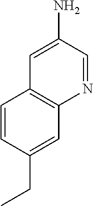

### Step 1: Preparation of 7-vinylquinolin-3-amine

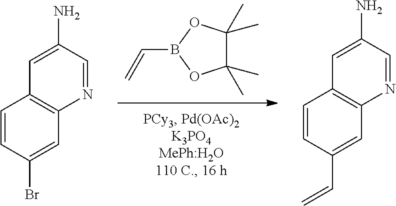

The title compound was prepared from 7-bromoquinolin-3-amine according to general procedure P. MS (m/z): [M+H]⁺: 170.9

### Step 2: Preparation of 7-ethylquinolin-3-amine

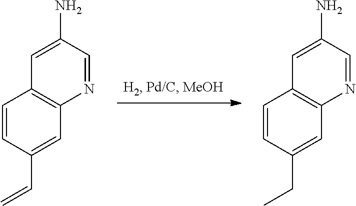

The title compound was prepared from 7-Vinylquinolin-3-amine according to general procedure P in MeOH. MS (m/z): [M+H]⁺: 172.9 Example 237: Preparation of Compound 72

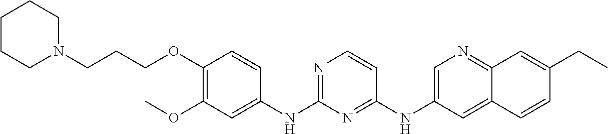 [SMILES: CCc1ccc2cc(Nc3ccnc(Nc4ccc(OCCCN5CCCCC5)c(OC)c4)n3)cnc2c1]

Compound 108 was prepared from the corresponding intermediate according to general procedure C. MS (m/z): [M+H]⁺ 513.5. H NMR (500 MHz, MeOD) δ 8.81 (d, J=2.2 Hz, 1H), 8.66 (d, J=2.4 Hz, 1H), 7.89 (s, 1H), 7.63 (s, 1H), 7.47 (d, J=8.4 Hz, 1H), 7.37-7.26 (m, 1H), 7.11 (d, J=2.3 Hz, 1H), 6.95 (dd, J=8.5, 2.4 Hz, 1H), 6.82 (d, J=8.6 Hz, 1H), 6.15 (d, J=5.8 Hz, 1H), 3.96 (t, J=6.2 Hz, 2H), 3.54 (s, 3H), 2.75 (q, J=7.6 Hz, 2H), 2.52-2.45 (m, 2H), 2.39 (s, 4H), 1.93 (td, J=12.1, 6.2 Hz, 2H), 1.53 (dt, J=11.1, 5.7 Hz, 4H), 1.39 (s, 2H), 1.25 (t, J=7.6 Hz, 3H).

### Example 238: Preparation of 2-methoxyquinolin-3-amine (Intermediate 101)

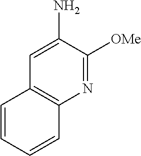 [SMILES: COc1nc2ccccc2cc1N]

### Step 1: Preparation of 2-methoxy-3-nitroquinoline

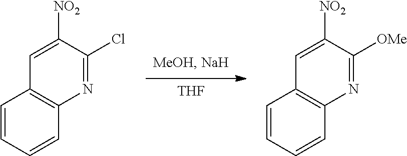 [SMILES: O=[N+]([O-])c1cc2ccccc2nc1Cl ; COc1nc2ccccc2cc1[N+](=O)[O-] ; "THF"]

To anhydrous MeOH (500 uL) under N₂ in an ice bath was added sodium hydride (60% dispersion in mineral oil) (1.5 equiv) and the reaction stirred for 15 minutes. The vial was removed from the ice and 2-chloro-3-nitroquinoline (20.0 mg, 91.1 umol) was added and continued stirring for 16 h. The reaction was then quenched with sat. NH₄Cl solution and extracted with EtOAc (3×) before drying and concentrating to deliver the title product. MS (m/z): [M+H]⁺: 204.8

### Step 2: Preparation of 2-methoxyquinolin-3-amine

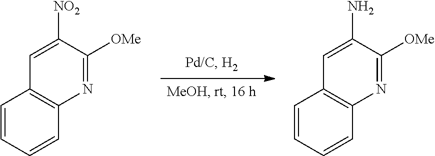 [SMILES: COc1nc2ccccc2cc1[N+](=O)[O-] ; "MeOH, rt, 16 h" ; COc1nc2ccccc2cc1N]

The title compound was prepared from 2-methoxy-3-nitroquinoline according to general procedure H. MS (m/z): [M+H]⁺: 174.7 Example 239: Preparation of Compound 108

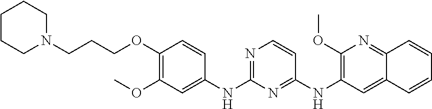 [SMILES: COc1cc(Nc2nccc(Nc3cc4ccccc4nc3OC)n2)ccc1OCCCN1CCCCC1]

Compound 108 was prepared from the corresponding intermediate according to general procedure C. MS (m/z): [M+H]⁺ 515.5. H NMR (500 MHz, DMSO) δ 9.16 (s, 1H), 9.09 (s, 1H), 8.95 (s, 1H), 8.08 (d, J=5.7 Hz, 1H), 7.73 (d, J=8.3 Hz, 1H), 7.68 (s, 1H), 7.54 (ddd, J=8.4, 7.0, 1.5 Hz, 1H), 7.44-7.37 (m, 1H), 7.32 (s, 1H), 7.26 (d, J=8.3 Hz, 1H), 6.87 (d, J=8.6 Hz, 1H), 6.64 (d, J=5.7 Hz, 1H), 4.12 (s, 3H), 4.00 (t, J=5.9 Hz, 2H), 3.64 (br s, 3H), 2.89 (br s, 4H), 2.08 (br s, 2H), 1.68 (br s, 4H), 1.45 (br s, 2H). *one CH2 peak missing due to overlap with solvent peak.

### Example 240: Preparation of 3-amino-6-chloroquinolin-2(1H)-one (Intermediate 102)

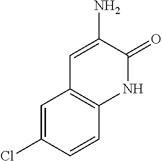 [SMILES: Nc1cc2cc(Cl)ccc2[nH]c1=O]

### Step 1: Preparation of 6-chloro-3-nitroquinolin-2(1H)-one

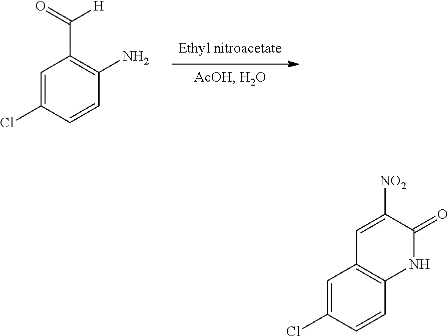

The title compound was prepared from 2-amino-5-chlorobenzaldehyde according to general procedure Q. MS (m/z): [M+H]⁺: 225.1

### Step 2: Preparation of 3-amino-6-chloroquinolin-2(1H)-one

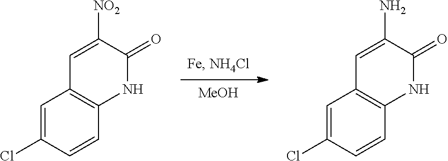 [SMILES: "MeOH" ; O=c1[nH]c2ccc(Cl)cc2cc1[N+](=O)[O-] ; Nc1cc2cc(Cl)ccc2[nH]c1=O]

The title compound was prepared from 6-chloro-3-nitroquinolin-2(1H)-one according to general procedure I. MS (m/z): [M+H]⁺: 195.0

### Example 241: Preparation of Compound 109

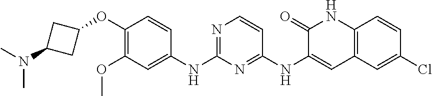 [SMILES: COc1cc(Nc2nccc(Nc3cc4cc(Cl)ccc4[nH]c3=O)n2)ccc1O[C@H]1C[C@H](N(C)C)C1]

Compound 109 was prepared from the corresponding intermediate according to general procedure C. MS (m/z): [M+H]⁺ 507.7. ¹H NMR (500 MHz, DMSO) δ 12.28 (s, 1H), 8.98 (d, J=8.3 Hz, 2H), 8.90 (s, 1H), 8.00 (d, J=5.7 Hz, 1H), 7.42 (s, 1H), 7.32 (dd, J=8.7, 2.3 Hz, 1H), 7.22 (d, J=8.7 Hz, 2H), 7.14 (d, J=7.7 Hz, 1H), 6.68 (d, J=5.7 Hz, 1H), 6.64 (d, J=8.7 Hz, 1H), 4.67-4.60 (m, 1H), 3.65 (s, 3H), 2.79 (s, 1H), 2.29-2.18 (m, 2H), 2.15-2.06 (m, 2H), 2.02 (s, 6H).

### Example 242: Preparation of 3-amino-7-chloroquinolin-2(1H)-one (Intermediate 103)

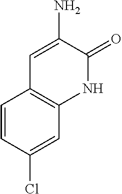 [SMILES: Nc1cc2ccc(Cl)cc2[nH]c1=O]

### Step 1: Preparation of 7-chloro-3-nitroquinolin-2(1H)-one

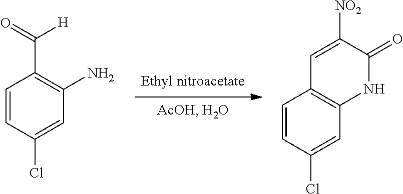

The title compound was prepared from 2-amino-4-chlorobenzaldehyde according to general procedure Q. MS (m/z): [M+H]⁺: 225.1

### Step 2: Preparation of 3-amino-7-chloroquinolin-2(1H)-one

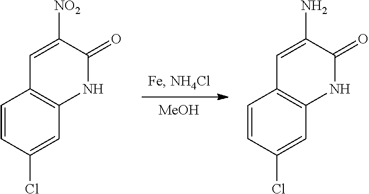 [SMILES: "MeOH" ; O=c1[nH]c2cc(Cl)ccc2cc1[N+](=O)[O-] ; Nc1cc2ccc(Cl)cc2[nH]c1=O]

The title compound was prepared from 7-chloro-3-nitroquinolin-2(1H)-one according to general procedure I. MS (m/z): [M+H]⁺: 195.1 Example 243: Preparation of Compound 110

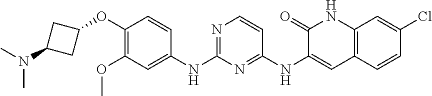 [SMILES: COc1cc(Nc2nccc(Nc3cc4ccc(Cl)cc4[nH]c3=O)n2)ccc1O[C@H]1C[C@H](N(C)C)C1]

Compound 110 was prepared from the corresponding intermediate according to general procedure C. MS (m/z): [M+H]⁺ 507.5. H NMR (500 MHz, DMSO) δ 9.15 (s, 1H), 9.11 (s, 1H), 9.00 (s, 1H), 8.14 (d, J=5.7 Hz, 1H), 7.49 (s, 1H), 7.36 (s, 2H), 7.30 (d, J=7.5 Hz, 1H), 7.25 (d, J=7.9 Hz, 1H), 6.80 (t, J=7.4 Hz, 2H), 4.81-4.74 (m, 1H), 3.79 (s, 3H), 2.95-2.88 (m, 1H), 2.43-2.36 (m, 2H), 2.29-2.22 (m, 2H), 2.17 (s, 6H).

### Example 244: Preparation of 3-amino-6-bromoquinolin-2(1H)-one (Intermediate 104)

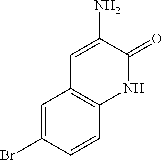 [SMILES: Nc1cc2cc(Br)ccc2[nH]c1=O]

### Step 1: Preparation of 6-bromo-3-nitroquinolin-2(1H)-one

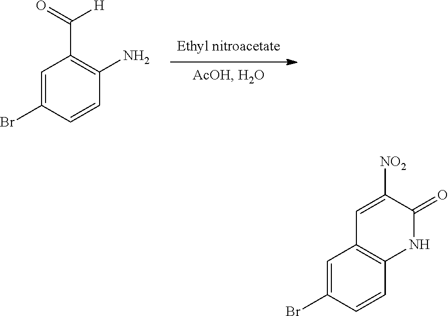

The title compound was prepared from 2-amino-5-bromobenzaldehyde according to general procedure Q. MS (m/z): [M+H]⁺: 268.9

### Step 2: Preparation of 3-amino-6-bromoquinolin-2(1H)-one

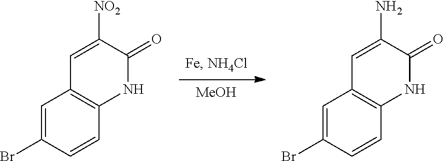 [SMILES: "MeOH" ; O=c1[nH]c2ccc(Br)cc2cc1[N+](=O)[O-] ; Nc1cc2cc(Br)ccc2[nH]c1=O]

The title compound was prepared from 6-bromo-3-nitroquinolin-2(1H)-one according to general procedure I. MS (m/z): [M+H]⁺: 239.0, 241.0 Example 245: Preparation of Compound 111

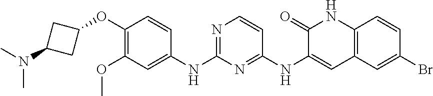 [SMILES: COc1cc(Nc2nccc(Nc3cc4cc(Br)ccc4[nH]c3=O)n2)ccc1O[C@H]1C[C@H](N(C)C)C1]

Compound 111 was prepared from the corresponding intermediate according to general procedure C. MS (m/z): [M+H]⁺ 551.4. H NMR (500 MHz, DMSO) δ 12.36 (s, 1H), 9.07 (d, J=8.8 Hz, 2H), 9.03-8.88 (m, 1H), 8.08 (d, J=5.7 Hz, 1H), 7.65 (s, 1H), 7.51 (dd, J=8.7, 2.0 Hz, 1H), 7.28 (s, 1H), 7.24 (d, J=8.7 Hz, 2H), 6.76 (d, J=5.7 Hz, 1H), 6.71 (d, J=8.6 Hz, 1H), 4.71 (s, 1H), 3.73 (s, 3H), 2.87-2.79 (m, 1H), 2.35-2.25 (m, 2H), 2.21-2.12 (m, 2H), 2.07 (s, 6H).

### Example 246: Preparation of 3-amino-8-chloroquinolin-2(1H)-one (Intermediate 105)

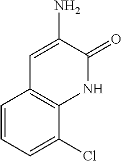 [SMILES: Nc1cc2cccc(Cl)c2[nH]c1=O]

### Step 1: Preparation of 8-chloro-3-nitroquinolin-2(1H)-one

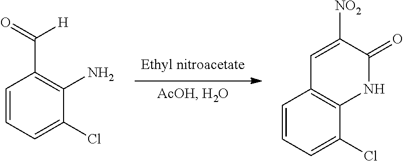

The title compound was prepared from 2-amino-3-chlorobenzaldehyde according to general procedure Q. MS (m/z): [M+H]⁺: 225.0

### Step 2: Preparation of 3-amino-8-chloroquinolin-2(1H)-one

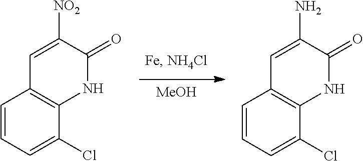 [SMILES: "MeOH" ; O=c1[nH]c2c(Cl)cccc2cc1[N+](=O)[O-] ; Nc1cc2cccc(Cl)c2[nH]c1=O]

The title compound was prepared from 8-chloro-3-nitroquinolin-2(1H)-one according to general procedure I. MS (m/z): [M+H]⁺: 195.1 Example 247: Preparation of Compound 112

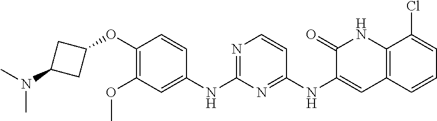 [SMILES: COc1cc(Nc2nccc(Nc3cc4cccc(Cl)c4[nH]c3=O)n2)ccc1O[C@H]1C[C@H](N(C)C)C1]

Compound 112 was prepared from the corresponding intermediate according to general procedure C. MS (m/z): [M+H]⁺ 507.4. H NMR (500 MHz, DMSO) δ 11.59 (s, 1H), 9.11 (d, J=8.2 Hz, 2H), 9.01 (s, 1H), 8.09 (d, J=5.7 Hz, 1H), 7.51 (d, J=7.9 Hz, 1H), 7.40 (s, 1H), 7.27 (s, 1H), 7.20 (t, J=7.9 Hz, 2H), 6.78 (d, J=5.7 Hz, 1H), 6.72 (d, J=8.7 Hz, 1H), 4.74-4.64 (m, 1H), 3.70 (s, 3H), 2.89-2.80 (m, 1H), 2.35-2.27 (m, 2H), 2.21-2.12 (m, 2H), 2.09 (s, 6H).

### Example 248: Preparation of 6,8-difluoroquinolin-3-amine (Intermediate 106)

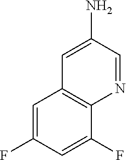 [SMILES: Nc1cnc2c(F)cc(F)cc2c1]

### Step 1: Preparation 3-bromo-6,8-difluoroquinoline

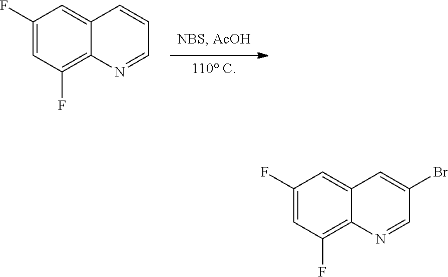 [SMILES: Fc1cc(F)c2ncccc2c1 ; "110° C." ; Fc1cc(F)c2ncc(Br)cc2c1]

The title compound was prepared from 6,8-difluoroquinoline according to general procedure W. MS (m/z): [M+H]⁺: 244.0

### Step 2: Preparation of 6,8-difluoroquinolin-3-amine

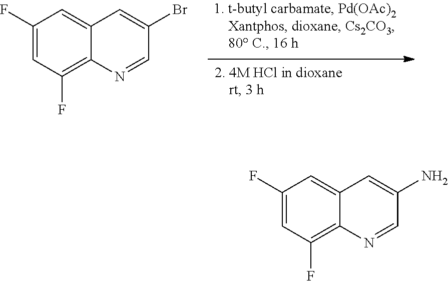 [SMILES: Fc1cc(F)c2ncc(Br)cc2c1 ; "2. 4M HCl in dioxane    rt, 3 h" ; Nc1cnc2c(F)cc(F)cc2c1]

The title compound was prepared from 3-bromo-6,8-difluoroquinoline according to general procedure X. MS (m/z): [M+H]⁺: 181.0

### Example 249: Preparation of Compound 113

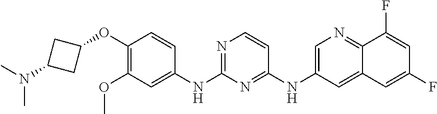 [SMILES: COc1cc(Nc2nccc(Nc3cnc4c(F)cc(F)cc4c3)n2)ccc1O[C@H]1C[C@@H](N(C)C)C1]

Compound 113 was prepared from the corresponding intermediate according to general procedure C. MS (m/z): [M+H]⁺ 493.3. H NMR (500 MHz, DMSO) δ 10.07 (s, 1H), 9.13 (s, 1H), 9.09 (s, 1H), 8.98 (s, 1H), 8.16 (d, J=5.6 Hz, 1H), 7.59-7.50 (m, 1H), 7.33 (s, 1H), 7.31-7.22 (m, 2H), 6.84 (d, J=8.6 Hz, 1H), 6.37 (d, J=5.6 Hz, 1H), 4.43-4.34 (m, 1H), 3.71 (s, 3H), 2.62 (m, 2H), 2.36 (m, 1H), 2.10 (s, 6H), 1.89 (m, 2H).

### Example 250: Preparation of 6-chloro-8-fluoroquinolin-3-amine (Intermediate 107)

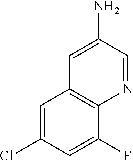 [SMILES: Nc1cnc2c(F)cc(Cl)cc2c1]

### Step 1: Preparation of 6-chloro-8-fluoroquinoline

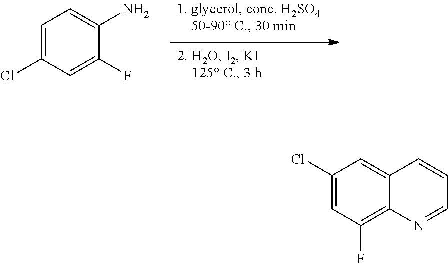 [SMILES: Nc1ccc(Cl)cc1F ; "2. H2O, I2, KI    125° C., 3 h" ; Fc1cc(Cl)cc2cccnc12]

The title compound was prepared from 4-chloro-2-fluoroaniline according to general procedure V. MS (m/z): [M+H]⁺: 182.0

### Step 2: Preparation of 3-bromo-6-chloro-8-fluoroquinoline

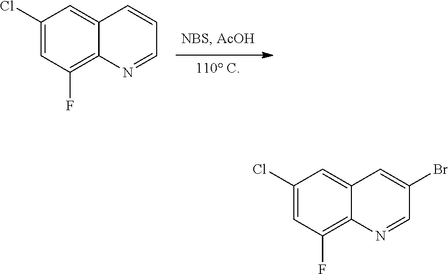 [SMILES: Fc1cc(Cl)cc2cccnc12 ; "110° C." ; Fc1cc(Cl)cc2cc(Br)cnc12]

The title compound was prepared from 6-chloro-8-fluoroquinoline according to general procedure W. MS (m/z): [M+H]⁺:260.0, 262.0.

### Step 3: Preparation of 6-chloro-8-fluoroquinolin-3-amine

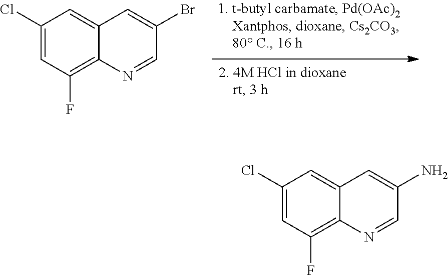 [SMILES: Fc1cc(Cl)cc2cc(Br)cnc12 ; "2. 4M HCl in dioxane    rt, 3 h" ; Nc1cnc2c(F)cc(Cl)cc2c1]

The title compound was prepared from 3-bromo-6-chloro-8-fluoroquinoline according to general procedure X. MS (m/z): [M+H]⁺: 197.1

### Example 251: Preparation of Compound 114

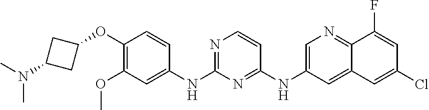 [SMILES: COc1cc(Nc2nccc(Nc3cnc4c(F)cc(Cl)cc4c3)n2)ccc1O[C@H]1C[C@@H](N(C)C)C1]

Compound was prepare from the corresponding intermediate according to general procedure C. MS (m/z): [M+H]⁺ 509.7. ¹H NMR (500 MHz, DMSO) δ 10.06 (s, 1H), 9.11 (s, 1H), 9.08 (s, 1H), 8.98 (s, 1H), 8.13 (d, J=5.7 Hz, 1H), 7.68 (s, 1H), 7.58 (dd, J=10.4, 2.2 Hz, 1H), 7.25 (d, J=8.4 Hz, 2H), 6.79 (d, J=8.5 Hz, 1H), 6.33 (d, J=5.7 Hz, 1H), 4.40-4.30 (m, 1H), 3.68 (s, 3H), 2.64-2.53 (m, 2H), 2.42-2.29 (m, 1H), 2.08 (s, 6H), 1.90-1.83 (m, 2H).

### Example 252: Preparation of 6,7-difluoroquinolin-3-amine (Intermediate 108)

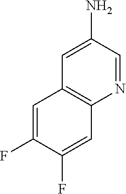 [SMILES: Nc1cnc2cc(F)c(F)cc2c1]

### Step 1: Preparation of 6,7-difluoroquinoline

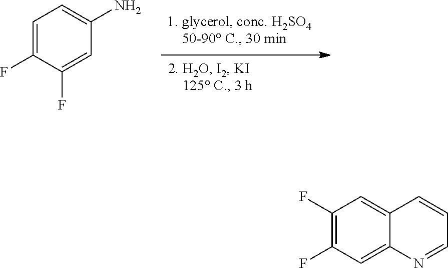 [SMILES: Nc1ccc(F)c(F)c1 ; "2. H2O, I2, KI    125° C., 3 h" ; Fc1cc2cccnc2cc1F]

The title compound was prepared from 3,4-difluoroaniline according to general procedure V. MS (m/z): [M+H]⁺: 166.1

### Step 2: Preparation of 3-bromo-6,7-difluoroquinoline

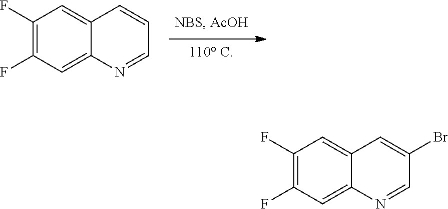 [SMILES: Fc1cc2cccnc2cc1F ; "110° C." ; Fc1cc2cc(Br)cnc2cc1F]

The title compound was prepared from 6,7-difluoroquinoline according to general procedure W. MS (m/z): [M+H]⁺: 244.0

### Step 3: Preparation of 6,7-difluoroquinolin-3-amine

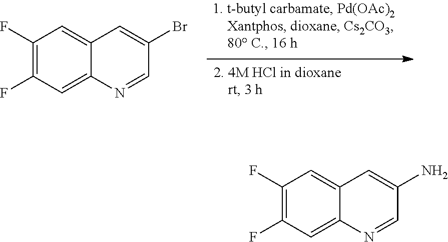 [SMILES: Fc1cc2cc(Br)cnc2cc1F ; "2. 4M HCl in dioxane    rt, 3 h" ; Nc1cnc2cc(F)c(F)cc2c1]

The title compound was prepared from 3-bromo-6,7-difluoroquinoline according to general procedure X. MS (m/z): [M+H]⁺: 181.1

### Example 253: Preparation of Compound 115

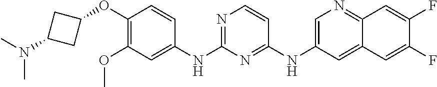 [SMILES: COc1cc(Nc2nccc(Nc3cnc4cc(F)c(F)cc4c3)n2)ccc1O[C@H]1C[C@@H](N(C)C)C1]

Compound 115 was prepared from the corresponding intermediate according to general procedure C. MS (m/z): [M+H]⁺ 493.3. ¹H NMR (500 MHz, DMSO) δ 9.91 (s, 1H), 9.06 (s, 1H), 8.98 (s, 1H), 8.96 (s, 1H), 8.10 (d, J=5.7 Hz, 1H), 7.94 (t, J=9.8 Hz, 1H), 7.68 (s, 1H), 7.31-7.16 (m, 2H), 6.78 (d, J=8.7 Hz, 1H), 6.30 (d, J=5.7 Hz, 1H), 4.34 (p, J=7.3 Hz, 1H), 3.65 (s, 3H), 2.59 (dtd, J=9.3, 6.7, 2.8 Hz, 2H), 2.36 (dd, J=6.8, 5.0 Hz, 1H), 2.09 (d, J=7.0 Hz, 6H), 1.90-1.80 (m, 2H).

### Example 254: Preparation of 8-chloro-6-fluoroquinolin-3-amine (Intermediate 109)

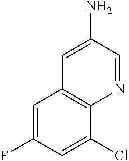 [SMILES: Nc1cnc2c(Cl)cc(F)cc2c1]

### Step 1: Preparation of 8-chloro-6-fluoroquinoline

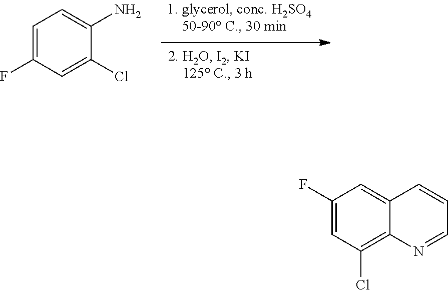 [SMILES: Nc1ccc(F)cc1Cl ; "2. H2O, I2, KI    125° C., 3 h" ; Fc1cc(Cl)c2ncccc2c1]

The title compound was prepared from 2-chloro-4-fluoroaniline according to general procedure V. MS (m/z): [M+H]⁺: 182.2

### Step 2: Preparation of 3-bromo-8-chloro-6-fluoroquinoline

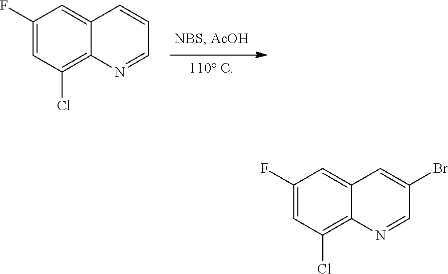 [SMILES: Fc1cc(Cl)c2ncccc2c1 ; "110° C." ; Fc1cc(Cl)c2ncc(Br)cc2c1]

The title compound was prepared from 8-chloro-6-fluoroquinoline according to general procedure W. MS (m/z): [M+H]⁺: 262.0

### Step 3: Preparation of 8-chloro-6-fluoroquinolin-3-amine

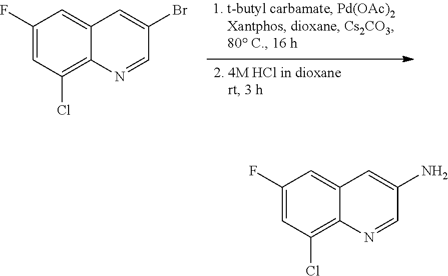 [SMILES: Fc1cc(Cl)c2ncc(Br)cc2c1 ; "2. 4M HCl in dioxane    rt, 3 h" ; Nc1cnc2c(Cl)cc(F)cc2c1]

The title compound was prepared from 3-bromo-8-chloro-6-fluoroquinoline according to general procedure X. MS (m/z): [M+H]⁺: 197.1

### Example 255: Preparation of Compound 116

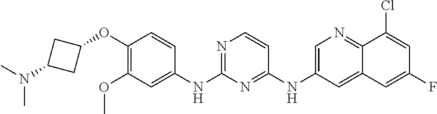 [SMILES: COc1cc(Nc2nccc(Nc3cnc4c(Cl)cc(F)cc4c3)n2)ccc1O[C@H]1C[C@@H](N(C)C)C1]

Compound 116 was prepared from the corresponding intermediate according to general procedure C. MS (m/z): [M+H]⁺ 509.3. ¹H NMR (500 MHz, DMSO) δ 10.10 (s, 1H), 9.15 (s, 1H), 9.08 (s, 2H), 8.17 (dd, J=5.6, 3.3 Hz, 1H), 7.85 (dd, J=8.6, 2.8 Hz, 1H), 7.49 (d, J=2.5 Hz, 1H), 7.36-7.22 (m, 2H), 6.84 (d, J=8.6 Hz, 1H), 6.38 (d, J=5.7 Hz, 1H), 4.43-4.33 (m, 1H), 3.71 (s, 3H), 2.66-2.59 (m, 2H), 2.39-2.31 (m, 1H), 2.09 (s, 6H), 1.93-1.84 (m, 2.7 Hz, 2H).

### Example 256: Preparation of 3-amino-7,8-difluoroquinolin-2(1H)-one (Intermediate 110)

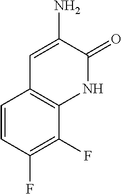 [SMILES: Nc1cc2ccc(F)c(F)c2[nH]c1=O]

### Step 1: Preparation of 2-amino-3,4-difluorobenzaldehyde

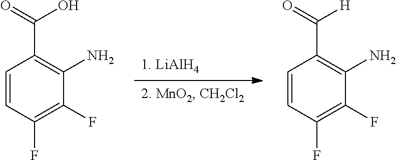 [SMILES: Nc1c(C(=O)O)ccc(F)c1F ; "2. MnO2, CH2Cl2" ; Nc1c(C=O)ccc(F)c1F]

The title compound was prepared from 2-amino-3,4-difluorobenzoic acid according to general procedure Y. ¹H NMR (500 MHz, CDCl₃) δ 9.75 (d, J=2.0 Hz, 1H), 7.22-7.19 (m, 1H), 6.51-6.45 (m, 1H).

### Step 2: Preparation of 7,8-difluoro-3-nitroquinolin-2(1H)-one

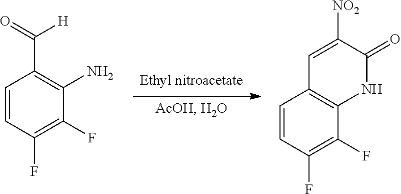

The title compound was prepared from 2-amino-3,4-difluorobenzaldehyde according to general procedure Q. MS (m/z): [M+H]⁺: 227.1

### Step 3: Preparation of 3-amino-7,8-difluoroquinolin-2(1H)-one

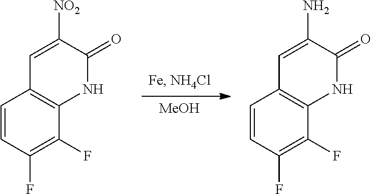 [SMILES: "MeOH" ; O=c1[nH]c2c(F)c(F)ccc2cc1[N+](=O)[O-] ; Nc1cc2ccc(F)c(F)c2[nH]c1=O]

The title compound was prepared from 7,8-difluoro-3-nitroquinolin-2(1H)-one according to general procedure I. MS (m/z): [M+H]⁺: 197.1

### Example 257: Preparation of Compound 117

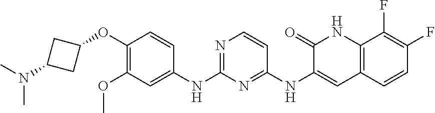 [SMILES: COc1cc(Nc2nccc(Nc3cc4ccc(F)c(F)c4[nH]c3=O)n2)ccc1O[C@H]1C[C@@H](N(C)C)C1]

Compound 117 was prepared from the corresponding intermediate according to general procedure C. MS (m/z): [M+H]⁺ 509.3. ¹H NMR (500 MHz, DMSO) δ 9.02 (d, J=3.1 Hz, 2H), 8.80 (s, 1H), 8.04 (d, J=5.7 Hz, 1H), 7.28 (s, 1H), 7.22 (d, J=7.4 Hz, 1H), 7.13 (s, 1H), 7.02 (s, 1H), 6.79 (d, J=8.6 Hz, 1H), 6.65 (d, J=5.1 Hz, 1H), 4.41-4.31 (m, 1H), 3.70 (s, 3H), 2.63-2.56 (m, 2H), 2.34 (dd, J=17.4, 10.0 Hz, 1H), 2.06 (s, 6H), 1.85 (dd, J=19.0, 8.5 Hz, 2H).

### Example 258: Preparation of 5,7-difluoroquinolin-3-amine (Intermediate 111)

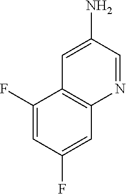 [SMILES: Nc1cnc2cc(F)cc(F)c2c1]

### Step 1: Preparation of 5,7-difluoroquinoline 1-oxide

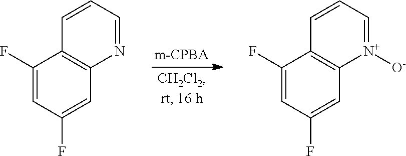 [SMILES: Fc1cc(F)c2cccnc2c1 ; [O-][n+]1cccc2c(F)cc(F)cc21]

To 5,7-difluoroquinoline (1.00 g, 5.75 mmol) in DCM (24 mL) cooled to 0° C. was added m-CPBA (1.70 g, 6.90 mmol) portionwise. The mixture was allowed to warm to room temperature and stirred overnight. The reaction was washed with sat. Na₂CO₃ (2×), then brine. The organic layer was dried over Na₂SO₄, filtered and concentrated to give 5,7-difluoroquinoline 1-oxide (884 mg, 66%) without any further purification. MS (m/z): [M+H]⁺: 182.1

### Step 2: Preparation of 5,7-difluoro-3-nitroquinoline 1-oxide

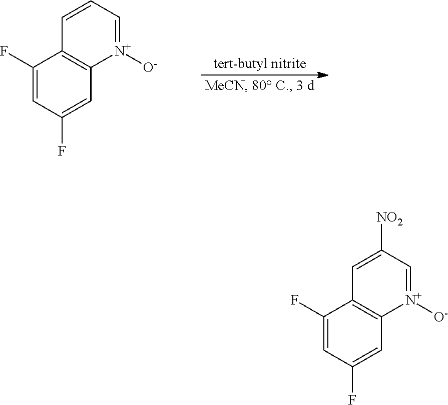 [SMILES: [O-][n+]1cccc2c(F)cc(F)cc21 ; O=[N+]([O-])c1cc2c(F)cc(F)cc2[n+]([O-])c1]

To 5,7-difluoroquinoline 1-oxide (884 mg, 4.88 mmol) in MeCN (19.5 mL) was added tert-butyl nitrite (2.58 mL, 19.5 mmol). The reaction mixture was heated to 80° C. and stirred for 3 d. The crude reaction was concentrated directly onto silica gel and purified by chromatography on a silica column, eluting with a solvent gradient of 0-10% MeOH (3% NH₄OH) in DCM to deliver the title compound (185 mg, 17%). MS (m/z): [M+H]⁺: 226.9

### Step 3: Preparation of 5,7-difluoroquinolin-3-amine

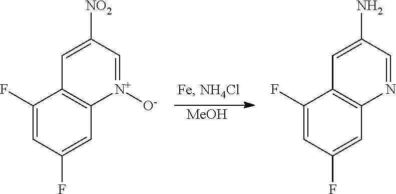 [SMILES: "MeOH" ; O=[N+]([O-])c1cc2c(F)cc(F)cc2[n+]([O-])c1 ; Nc1cnc2cc(F)cc(F)c2c1]

The title compound was prepared from 5,7-difluoro-3-nitroquinoline 1-oxide according to general procedure I. MS (m/z): [M+H]⁺: 181.1

### Example 259: Preparation of Compound 118

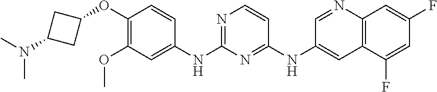 [SMILES: COc1cc(Nc2nccc(Nc3cnc4cc(F)cc(F)c4c3)n2)ccc1O[C@H]1C[C@@H](N(C)C)C1]

Compound 118 was prepared from the corresponding intermediate according to general procedure C. MS (m/z): [M+H]⁺ 493.3. H NMR (400 MHz, dmso) δ 9.86 (s, 1H), 9.16 (s, 1H), 9.05 (s, 1H), 8.68 (d, J=2.1 Hz, 1H), 8.06 (d, J=5.6 Hz, 1H), 7.56 (d, J=10.0 Hz, 1H), 7.53-7.44 (m, 1H), 7.21 (s, 1H), 7.16 (dd, J=8.6, 2.3 Hz, 1H), 6.64 (d, J=8.7 Hz, 1H), 6.24 (d, J=5.7 Hz, 1H), 4.31-4.20 (m, 1H), 3.54 (s, 3H), 2.51 (dd, J=9.0, 2.5 Hz, 2H), 2.28 (s, 1H), 2.03 (s, 6H), 1.78 (dd, J=19.0, 8.8 Hz, 2H).

### Example 260: Preparation of 3-amino-6,7-difluoroquinolin-2(1H)-one (Intermediate 112)

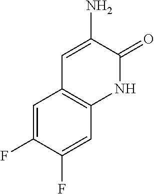 [SMILES: Nc1cc2cc(F)c(F)cc2[nH]c1=O]

### Step 1: Preparation of 2-amino-4,5-difluorobenzaldehyde

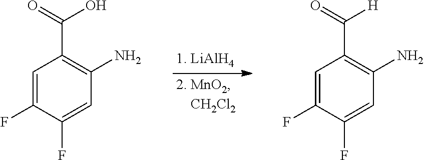 [SMILES: Nc1cc(F)c(F)cc1C(=O)O ; Nc1cc(F)c(F)cc1C=O]

The title compound was prepared from 2-amino-4,5-difluorobenzoic acid according to general procedure Y.

### Step 2: Preparation of 6,7-difluoro-3-nitroquinolin-2(1H)-one

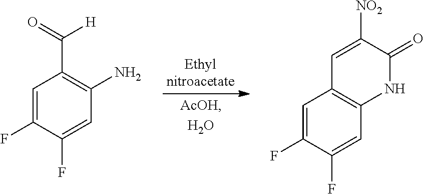 [SMILES: Nc1cc(F)c(F)cc1C=O ; O=c1[nH]c2cc(F)c(F)cc2cc1[N+](=O)[O-]]

The title compound was prepared from 2-amino-4,5-difluorobenzaldehyde according to general procedure Q. MS (m/z): [M+H]⁺: 227.0

### Step 3: Preparation of 3-amino-6,7-difluoroquinolin-2(1H)-one

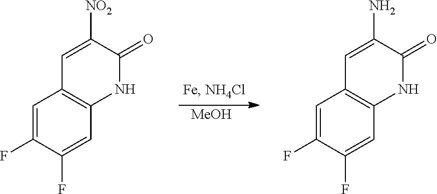 [SMILES: "MeOH" ; O=c1[nH]c2cc(F)c(F)cc2cc1[N+](=O)[O-] ; Nc1cc2cc(F)c(F)cc2[nH]c1=O]

The title compound was prepared from 6,7-difluoro-3-nitroquinolin-2(1H)-one according to general procedure I. MS (m/z): [M+H]⁺: 197.0

### Example 261: Preparation of Compound 119

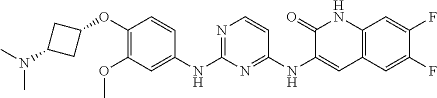 [SMILES: COc1cc(Nc2nccc(Nc3cc4cc(F)c(F)cc4[nH]c3=O)n2)ccc1O[C@H]1C[C@@H](N(C)C)C1]

Compound 119 was prepared from the corresponding intermediate according to general procedure C. MS (m/z): [M+H]⁺ 509.4. ¹H NMR (500 MHz, DMSO) δ 12.33 (s, 1H), 9.05 (s, 1H), 9.02 (s, 1H), 8.93 (s, 1H), 8.07 (d, J=5.7 Hz, 1H), 7.35 (s, 1H), 7.28-7.17 (m, 3H), 6.82 (d, J=8.7 Hz, 1H), 6.74 (d, J=5.8 Hz, 1H), 4.35 (dt, J=14.5, 7.2 Hz, 1H), 3.70 (s, 3H), 2.63-2.55 (m, 2H), 2.32 (dt, J=15.2, 7.7 Hz, 1H), 2.06 (s, 6H), 1.90-1.80 (m, 2H).

### Example 262: Preparation of 3-amino-8-fluoroquinolin-2(1H)-one (Intermediate 113)

### Step 1: Preparation of 8-fluoro-3-nitroquinolin-2(1H)-one

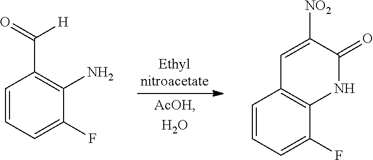 [SMILES: Nc1c(F)cccc1C=O ; O=c1[nH]c2c(F)cccc2cc1[N+](=O)[O-]]

The title compound was prepared from 2-amino-3-fluorobenzaldehyde according to general procedure Q. MS (m/z): [M+H]⁺: 209.1

### Step 2: Preparation of 3-amino-8-fluoroquinolin-2(1H)-one

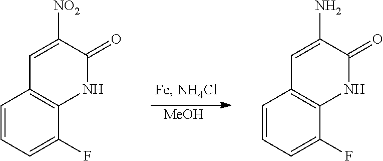 [SMILES: "MeOH" ; O=c1[nH]c2c(F)cccc2cc1[N+](=O)[O-] ; Nc1cc2cccc(F)c2[nH]c1=O]

The title compound was prepared from 8-fluoro-3-nitroquinolin-2(1H)-one according to general procedure I. MS (m/z): [M+H]⁺: 179.1

### Example 263: Preparation of Compound 120

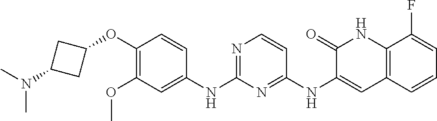 [SMILES: COc1cc(Nc2nccc(Nc3cc4cccc(F)c4[nH]c3=O)n2)ccc1O[C@H]1C[C@@H](N(C)C)C1]

Compound 120 was prepared from the corresponding intermediate according to general procedure C. MS (m/z): [M+H]⁺ 491.3. ¹H NMR (500 MHz, DMSO) δ 12.29 (s, 1H), 9.08 (d, J=13.6 Hz, 2H), 9.00 (s, 1H), 8.08 (d, J=5.7 Hz, 1H), 7.26 (dd, J=10.9, 9.1 Hz, 2H), 7.21 (d, J=7.5 Hz, 1H), 7.15 (dd, J=12.7, 7.8 Hz, 1H), 6.80 (d, J=8.7 Hz, 1H), 6.77 (d, J=5.7 Hz, 1H), 4.41-4.31 (m, 1H), 3.69 (s, 3H), 2.64-2.56 (m, 2H), 2.37 (d, J=1.9 Hz, 1H), 2.09 (s, 6H), 1.87 (dd, J=19.0, 8.4 Hz, 2H).

### Example 264: Preparation of 3-amino-6-fluoroquinolin-2(1H)-one (Intermediate 114)

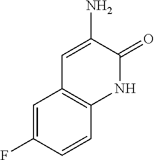 [SMILES: Nc1cc2cc(F)ccc2[nH]c1=O]

### Step 1: Preparation of 2-amino-5-fluorobenzaldehyde

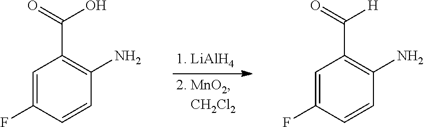 [SMILES: Nc1ccc(F)cc1C(=O)O ; Nc1ccc(F)cc1C=O]

The title compound was prepared from 2-amino-4,5-difluorobenzoic acid according to general procedure Y. ¹H NMR (500 MHz, DMSO) δ 9.82-9.76 (m, 1H), 7.40 (dd, J=9.1, 3.1 Hz, 1H), 7.23 (ddd, J=9.0, 8.4, 3.1 Hz, 1H), 7.01 (s, 2H), 6.79 (dd, J=9.1, 4.5 Hz, 1H).

### Step 2: Preparation of 6-fluoro-3-nitroquinolin-2(1H)-one

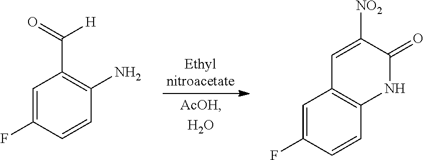 [SMILES: Nc1ccc(F)cc1C=O ; O=c1[nH]c2ccc(F)cc2cc1[N+](=O)[O-]]

The title compound was prepared from 2-amino-5-fluorobenzaldehyde according to general procedure Q. MS (m/z): [M+H]⁺: 209.1

### Step 3: Preparation of 3-amino-6-fluoroquinolin-2(1H)-one

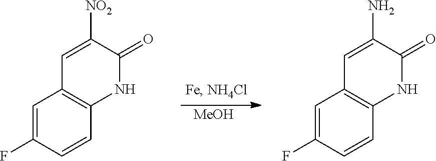 [SMILES: "MeOH" ; O=c1[nH]c2ccc(F)cc2cc1[N+](=O)[O-] ; Nc1cc2cc(F)ccc2[nH]c1=O]

The title compound was prepared from 6-fluoro-3-nitroquinolin-2(1H)-one according to general procedure I. MS (m/z): [M+H]⁺: 179.1

### Example 265: Preparation of Compound 121

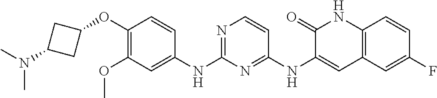 [SMILES: COc1cc(Nc2nccc(Nc3cc4cc(F)ccc4[nH]c3=O)n2)ccc1O[C@H]1C[C@@H](N(C)C)C1]

Compound 121 was prepared from the corresponding intermediate according to general procedure C. MS (m/z): [M+H]⁺ 491.3. ¹H NMR (500 MHz, DMSO) δ 12.22 (s, 1H), 8.98 (s, 2H), 8.87 (s, 1H), 8.01 (d, J=5.7 Hz, 1H), 7.23 (dd, J=8.9, 5.0 Hz, 1H), 7.20-7.11 (m, 3H), 7.10-7.03 (m, 1H), 6.75 (d, J=8.6 Hz, 1H), 6.68 (d, J=5.7 Hz, 1H), 4.33-4.23 (m, 1H), 3.63 (s, 3H), 2.55-2.47 (m, 2H), 2.25 (dt, J=15.0, 7.5 Hz, 1H), 1.99 (s, 6H), 1.78 (ddd, J=11.1, 8.4, 2.5 Hz, 2H).

### Example 266: Preparation of 3-amino-7-fluoroquinolin-2(1H)-one (Intermediate 115)

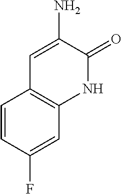 [SMILES: Nc1cc2ccc(F)cc2[nH]c1=O]

### Step 1: Preparation of 7-fluoro-3-nitroquinolin-2(1H)-one

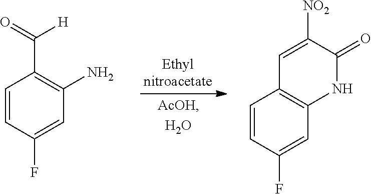 [SMILES: Nc1cc(F)ccc1C=O ; O=c1[nH]c2cc(F)ccc2cc1[N+](=O)[O-]]

The title compound was prepared from 2-amino-4-fluorobenzaldehyde according to general procedure Q. MS (m/z): [M+H]⁺: 209.1

### Step 2: Preparation of 3-amino-7-fluoroquinolin-2(1H)-one

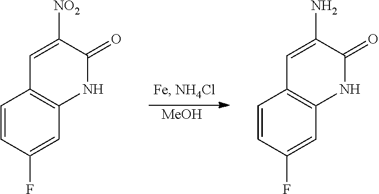 [SMILES: "MeOH" ; O=c1[nH]c2cc(F)ccc2cc1[N+](=O)[O-] ; Nc1cc2ccc(F)cc2[nH]c1=O]

The title compound was prepared from 7-fluoro-3-nitroquinolin-2(1H)-one according to general procedure I. MS (m/z): [M+H]⁺: 179.0

### Example 267: Preparation of Compound 122

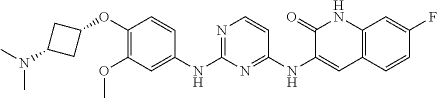 [SMILES: COc1cc(Nc2nccc(Nc3cc4ccc(F)cc4[nH]c3=O)n2)ccc1O[C@H]1C[C@@H](N(C)C)C1]

Compound 122 was prepared from the corresponding intermediate according to general procedure C. MS (m/z): [M+H]⁺ 491.3. H NMR (500 MHz, DMSO) δ 12.31 (s, 1H), 9.06 (s, 1H), 8.95 (s, 2H), 8.06 (d, J=5.7 Hz, 1H), 7.48 (s, 1H), 7.27 (s, 1H), 7.22 (d, J=7.5 Hz, 1H), 7.06 (t, J=8.2 Hz, 1H), 6.79 (d, J=8.7 Hz, 1H), 6.72 (d, J=5.7 Hz, 1H), 4.42-4.31 (m, 1H), 3.69 (s, 3H), 2.67-2.55 (m, 2H), 2.37 (s, 1H), 2.09 (s, 6H), 1.86 (d, J=9.7 Hz, 2H).

### Example 268: Preparation of 3-amino-5-fluoroquinolin-2(1H)-one (Intermediate 116)

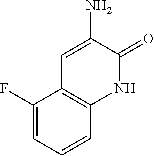 [SMILES: Nc1cc2c(F)cccc2[nH]c1=O]

### Step 1: Preparation of 5-fluoro-3-nitroquinolin-2(1H)-one

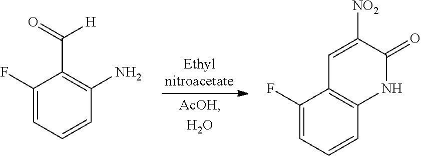 [SMILES: Nc1cccc(F)c1C=O ; O=c1[nH]c2cccc(F)c2cc1[N+](=O)[O-]]

The title compound was prepared from 2-amino-6-fluorobenzaldehyde according to general procedure Q. MS (m/z): [M+H]⁺: 209.1

### Step 2: Preparation of 3-amino-5-fluoroquinolin-2(1H)-one

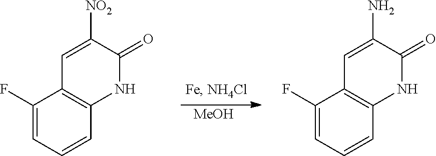 [SMILES: "MeOH" ; O=c1[nH]c2cccc(F)c2cc1[N+](=O)[O-] ; Nc1cc2c(F)cccc2[nH]c1=O]

The title compound was prepared from 5-fluoro-3-nitroquinolin-2(1H)-one according to general procedure I. MS (m/z): [M+H]⁺: 179.1

### Example 269: Preparation of Compound 123

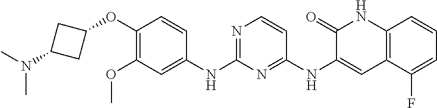 [SMILES: COc1cc(Nc2nccc(Nc3cc4c(F)cccc4[nH]c3=O)n2)ccc1O[C@H]1C[C@@H](N(C)C)C1]

Compound 123 was prepared from the corresponding intermediate according to general procedure D. MS (m/z): [M+H]⁺ 490.4. ¹H NMR (500 MHz, DMSO) δ 11.56 (s, 1H), 9.03 (s, 2H), 8.15 (s, 1H), 8.00 (d, J=5.7 Hz, 1H), 7.81 (s, 1H), 7.28 (d, J=8.8 Hz, 1H), 7.23 (s, 1H), 7.10 (d, J=7.8 Hz, 1H), 6.70 (d, J=8.7 Hz, 1H), 6.15 (d, J=5.7 Hz, 1H), 4.32-4.21 (m, 1H), 3.63 (s, 3H), 2.50 (m, 2H), 2.30-2.20 (m, 1H), 2.05 (td, J=8.1, 4.1 Hz, 1H), 1.98 (s, 6H), 1.75 (m, 2H), 0.96-0.89 (m, 2H).

### Example 270: Preparation of 3-amino-6-cyclopropylpicolinamide (Intermediate 117)

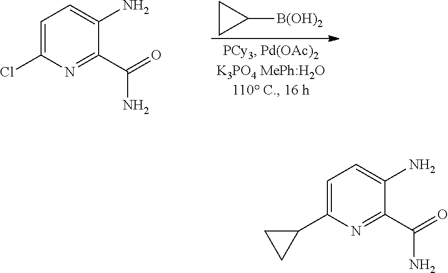 [SMILES: NC(=O)c1nc(Cl)ccc1N ; OB(O)C1CC1 ; NC(=O)c1nc(C2CC2)ccc1N]

The title compound was prepared in from 3-amino-6-chloropicolinamide and cyclopropylboronic acid according to general procedure P. MS (m/z): [M+H]⁺: 178.1

### Example 271: Preparation of Compound 124

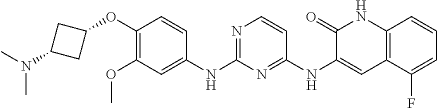 [SMILES: COc1cc(Nc2nccc(Nc3cc4c(F)cccc4[nH]c3=O)n2)ccc1O[C@H]1C[C@@H](N(C)C)C1]

Compound 124 was prepared from the corresponding intermediate according to general procedure C. MS (m/z): [M+H]⁺ 491.3. ¹H NMR (500 MHz, DMSO) δ 12.46 (s, 1H), 9.07 (d, J=18.6 Hz, 2H), 8.82 (s, 1H), 8.09 (d, J=5.6 Hz, 1H), 7.36 (dd, J=13.9, 8.0 Hz, 1H), 7.23 (d, J=8.7 Hz, 2H), 7.13 (d, J=8.3 Hz, 1H), 7.01-6.95 (m, 1H), 6.73 (dd, J=12.0, 7.0 Hz, 2H), 4.32 (p, J=7.1 Hz, 1H), 3.64 (s, 3H), 2.60-2.53 (m, 2H), 2.39-2.31 (m, 1H), 2.08 (s, 6H), 1.84 (dd, J=17.2, 6.6 Hz, 2H).

### Example 272: Preparation of 5,6-difluoroquinolin-3-amine (Intermediate 118)

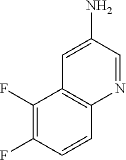 [SMILES: Nc1cnc2ccc(F)c(F)c2c1]

### Step 1: Preparation of 8-bromo-5,6-difluoroquinoline

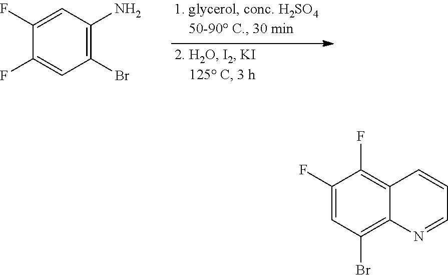 [SMILES: Nc1cc(F)c(F)cc1Br ; Fc1cc(Br)c2ncccc2c1F]

The title compound was prepared from 2-bromo-4,5-difluoroaniline according to general procedure V. MS (m/z): [M+H]⁺: 243.9, 245.9

### Step 2: Preparation of 5,6-difluoroquinoline

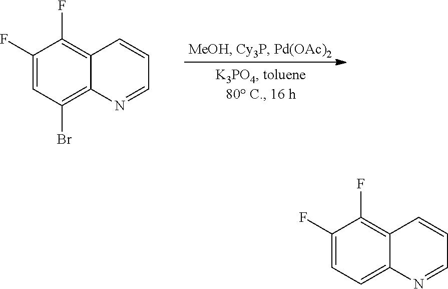 [SMILES: Fc1cc(Br)c2ncccc2c1F ; Fc1ccc2ncccc2c1F]

with a solution of 8-bromo-5,6-difluoroquinoline (1.00 g, 4.10 mmol), palladium(II) acetate (92.9 mg, 410 umol), tricyclohexylphosphine (244 mg, 820 umol), and potassium phosphate tribasic (1.33 g, 6.15 mmol) in anhydrous toluene (16 mL) and methanol (5 mL, 123 mmol) was stirred at 80° C. for 16 h. The reaction mixture was diluted with EtOAc and filtered through a pad of Celite. The filtrate was concentrated under reduced pressure and the crude product purified by flash column chromatography on silica gel column, eluting with a solvent gradient of 0-25% EtOAc/hexanes to afford the title product (478 mg, 71%).

### Step 3: Preparation of 3-bromo-5,6-difluoroquinoline

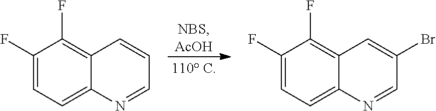 [SMILES: Fc1ccc2ncccc2c1F ; Fc1ccc2ncc(Br)cc2c1F]

The title compound was prepared from 5,6-difluoroquinoline according to general procedure W. MS (m/z): [M+H]⁺: 244.0

### Step 4: Preparation of 5,6-difluoroquinolin-3-amine

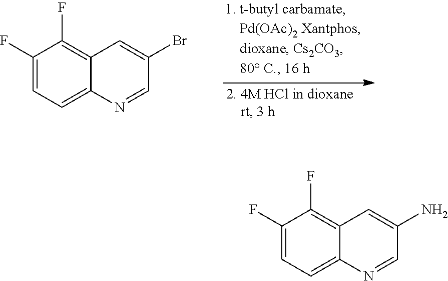 [SMILES: Fc1ccc2ncc(Br)cc2c1F ; Nc1cnc2ccc(F)c(F)c2c1]

The title compound was prepared from 3-bromo-5,6-difluoroquinoline according to general procedure X. MS (m/z): [M+H]⁺: 181.1

### Example 273: Preparation of Compound 125

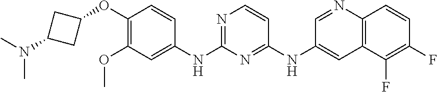 [SMILES: COc1cc(Nc2nccc(Nc3cnc4ccc(F)c(F)c4c3)n2)ccc1O[C@H]1C[C@@H](N(C)C)C1]

Compound 125 was prepared from the corresponding intermediate according to general procedure C. MS (m/z): [M+H]⁺ 493.4. ¹H NMR (500 MHz, DMSO) δ 10.01 (s, 1H), 9.14 (s, 1H), 9.09 (s, 1H), 8.86 (d, J=2.2 Hz, 1H), 8.13 (d, J=5.6 Hz, 1H), 7.84 (dd, J=9.2, 4.5 Hz, 1H), 7.71-7.63 (m, 1H), 7.28-7.19 (m, 2H), 6.73 (d, J=8.6 Hz, 1H), 6.33 (d, J=5.6 Hz, 1H), 4.35-4.27 (m, 1H), 3.61 (s, 3H), 2.56 (dtd, J=9.3, 6.7, 2.8 Hz, 2H), 2.35-2.27 (m, 1H), 2.06 (s, 6H), 1.83 (ddd, J=16.3, 8.5, 2.8 Hz, 2H).

### Example 274: Preparation of 6-((1R,5S)-8-oxa-3-azabicyclo[3.2.1]octan-3-yl)-5-methoxypyridin-3-amine (Intermediate 119)

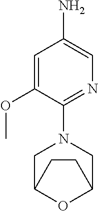 [SMILES: COc1cc(N)cnc1N1CC2CCC(C1)O2]

### Step 1: Preparation of (1R,5S)-3-(3-methoxy-5-nitropyridin-2-yl)-8-oxa-3-azabicyclo[3.2.1]octane

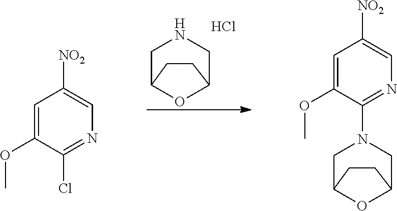 [SMILES: COc1cc([N+](=O)[O-])cnc1Cl ; C1CC2CNCC1O2 ; Cl ; COc1cc([N+](=O)[O-])cnc1N1CC2CCC(C1)O2]

The title compound was prepared from 2-chloro-3-methoxy-5-nitropyridine and 8-oxa-3-azabicyclo[3.2.1]octane hydrochloride according to general procedure G. MS (m/z): [M+H]⁺: 266.0

### Step 2: Preparation of 6-((1R,5S)-8-oxa-3-azabicyclo[3.2.1]octan-3-yl)-5-methoxypyridin-3-amine

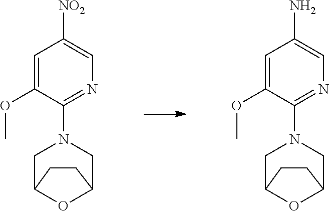 [SMILES: COc1cc([N+](=O)[O-])cnc1N1CC2CCC(C1)O2 ; COc1cc(N)cnc1N1CC2CCC(C1)O2]

The title compound was prepared from (1R,5S)-3-(3-methoxy-5-nitropyridin-2-yl)-8-oxa-3-azabicyclo[3.2.1]octane according to general procedure I. MS (m/z): [M+H]⁺: 236.2

### Example 275: Preparation of Compound 126

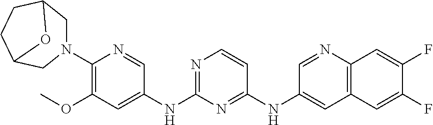 [SMILES: COc1cc(Nc2nccc(Nc3cnc4cc(F)c(F)cc4c3)n2)cnc1N1CC2CCC(C1)O2]

Compound 126 was prepared from the corresponding intermediate according to general procedure C. MS (m/z): [M+H]⁺ 492.4. H NMR (500 MHz, DMSO) δ 9.98 (s, 1H), 9.14 (s, 1H), 9.05 (s, 1H), 8.91 (s, 1H), 8.17 (d, J=2.1 Hz, 1H), 8.11 (d, J=5.7 Hz, 1H), 7.94 (dd, J=11.5, 8.0 Hz, 1H), 7.74 (s, 1H), 7.50 (s, 1H), 6.33 (d, J=5.7 Hz, 1H), 4.36 (s, 2H), 3.69 (s, 3H), 3.53 (d, J=11.5 Hz, 2H), 2.97 (d, J=11.2 Hz, 2H), 2.02-1.94 (m, 2H), 1.87-1.79 (m, 2H).

### Example 276: Preparation of 6-((1R,4R)-2-oxa-5-azabicyclo[2.2.1]heptan-5-yl)-5-methoxypyridin-3-amine (Intermediate 120)

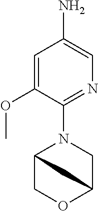 [SMILES: COc1cc(N)cnc1N1C[C@H]2C[C@@H]1CO2]

### Step 1: Preparation of (1R,4R)-5-(3-methoxy-5-nitropyridin-2-yl)-2-oxa-5-azabicyclo[2.2.1]heptane

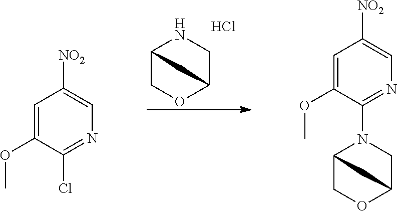 [SMILES: COc1cc([N+](=O)[O-])cnc1Cl ; C1O[C@H]2CN[C@@H]1C2 ; Cl ; COc1cc([N+](=O)[O-])cnc1N1C[C@H]2C[C@@H]1CO2]

The title compound was prepared from 2-chloro-3-methoxy-5-nitropyridine and (1R,4R)-2-oxa-5-azabicyclo[2.2.1]heptane hydrochloride according to general procedure G. MS (m/z): [M+H]⁺: 252.1

### Step 2: Preparation of 6-((1R,4R)-2-oxa-5-azabicyclo[2.2.1]heptan-5-yl)-5-methoxypyridin-3-amine

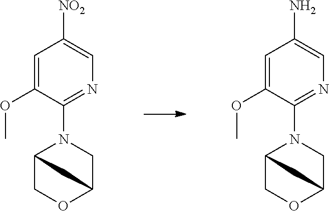 [SMILES: COc1cc([N+](=O)[O-])cnc1N1C[C@H]2C[C@@H]1CO2 ; COc1cc(N)cnc1N1C[C@H]2C[C@@H]1CO2]

The title compound was prepared from (1R,4R)-5-(3-methoxy-5-nitropyridin-2-yl)-2-oxa-5-azabicyclo[2.2.1]heptane according to general procedure I. MS (m/z): [M+H]⁺: 222.2

### Example 277: Preparation of Compound 127

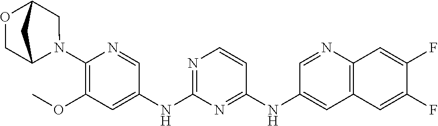 [SMILES: COc1cc(Nc2nccc(Nc3cnc4cc(F)c(F)cc4c3)n2)cnc1N1C[C@@H]2C[C@H]1CO2]

Compound 127 was prepared from the corresponding intermediate according to general procedure C. MS (m/z): [M+H]⁺ 478.6. ¹H NMR (500 MHz, DMSO) δ 9.92 (s, 1H), 9.00 (s, 2H), 8.91 (s, 1H), 8.09 (d, J=5.7 Hz, 1H), 8.05 (d, J=2.0 Hz, 1H), 7.93 (dd, J=11.4, 8.2 Hz, 1H), 7.70 (s, 1H), 7.41 (s, 1H), 6.30 (d, J=5.7 Hz, 1H), 4.84 (s, 1H), 4.56 (s, 1H), 3.86 (d, J=7.3 Hz, 1H), 3.77 (dd, J=7.3, 1.7 Hz, 1H), 3.70-3.62 (m, 4H) 3.37 (d, J=10.4 Hz, 1H), 1.86 (dd, J=9.5, 2.0 Hz, 1H), 1.79 (d, J=9.6 Hz, 1H).

### Example 278: Preparation of N2-(4-((1s,3s)-3-(dimethylamino)cyclobutoxy)-3-methoxyphenyl)-N4-(6-(trifluoromethoxy)quinolin-3-yl)pyrimidine-2,4-diamine (Compound 128)

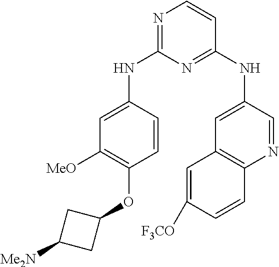 [SMILES: COc1cc(Nc2nccc(Nc3cnc4ccc(OC(F)(F)F)cc4c3)n2)ccc1O[C@H]1C[C@@H](N(C)C)C1]

The title compound was prepared according to the general SNAr procedure C with 6-(trifluoromethoxy)quinolin-3-amine. MS (m/z): [M+H]⁺−541.4. ¹H NMR (500 MHz, DMSO) δ 8.99 (d, J=2.6 Hz, 1H), 8.85 (s, 1H), 8.18 (d, J=5.7 Hz, 1H), 8.05-7.96 (m, 2H), 7.82 (d, J=8.2 Hz, 1H), 7.73 (s, 1H), 7.70-7.64 (m, 1H), 7.64-7.58 (m, 1H), 7.03 (d, J=8.9 Hz, 1H), 6.76-6.69 (m, 1H), 6.42 (d, J=5.7 Hz, 1H), 3.86 (s, 3H), 3.26 (s, 1H), 3.00-2.95 (m, 4H), 2.29 (s, 4H), 2.17 (s, 3H).

### Example 279: Preparation of Compound 129

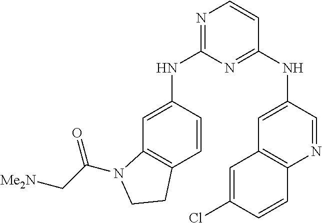 [SMILES: CN(C)CC(=O)N1CCc2ccc(Nc3nccc(Nc4cnc5ccc(Cl)cc5c4)n3)cc21]

### Step 1—Preparation of Intermediate 2-(dimethylamino)-1-(6-nitroindolin-1-yl)ethan-1-one

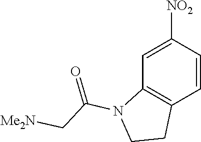 [SMILES: CN(C)CC(=O)N1CCc2ccc([N+](=O)[O-])cc21]

The title compound was prepared according to the general procedures J and K starting from 6-nitroindoline. MS (m/z): [M+H]⁺−250.2.

### Step 2—Preparation of Intermediate 1-(6-aminoindolin-1-yl)-2-(dimethylamino)ethan-1-one

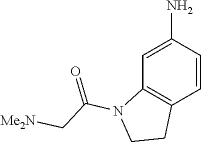 [SMILES: CN(C)CC(=O)N1CCc2ccc(N)cc21]

The title compound was prepared according to the general procedure H. MS (m/z): [M+H]⁺−220.1.

### Step 3—Preparation of Intermediate 1-(6-((4-chloropyrimidin-2-yl)amino)indolin-1-yl)-2-(dimethylamino)ethan-1-one

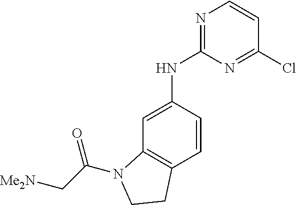 [SMILES: CN(C)CC(=O)N1CCc2ccc(Nc3nccc(Cl)n3)cc21]

The title compound was prepared according to the general procedure A. MS (m/z): [M+H]⁺−332.1.

### Step 4—Preparation of 1-(6-((4-((6-chloroquinolin-3-yl)amino)pyrimidin-2-yl)amino)indolin-1-yl)-2-(dimethylamino)ethan-1-one

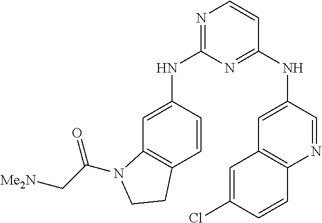 [SMILES: CN(C)CC(=O)N1CCc2ccc(Nc3nccc(Nc4cnc5ccc(Cl)cc5c4)n3)cc21]

The title compound was prepared according to the general SNAr procedure C. MS (m/z): [M+H]⁺474.4. H NMR (500 MHz, DMSO) δ 9.99 (s, 1H), 9.26 (s, 1H), 9.15 (s, 1H), 8.86 (s, 1H), 8.41 (s, 1H), 8.31 (br, 1H), 8.11 (d, J=5.6 Hz, 1H), 7.90 (d, J=8.9 Hz, 1H), 7.62 (br, 1H), 7.56 (d, J=6.6 Hz, 1H), 7.36 (br, 1H), 7.18 (d, J=8.0 Hz, 1H), 6.34 (d, J=5.7 Hz, 1H), 4.22 (t, J=8.4 Hz, 2H), 3.20-3.10 (m, 4H), 2.10 (s, 6H).

### Example 280: Preparation of 1-(6-((4-((6-bromoquinolin-3-yl)amino)pyrimidin-2-yl)amino)indolin-1-yl)-2-(dimethylamino)ethan-1-one (Compound 130)

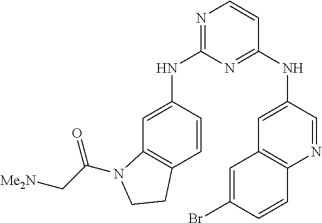 [SMILES: CN(C)CC(=O)N1CCc2ccc(Nc3nccc(Nc4cnc5ccc(Br)cc5c4)n3)cc21]

The title compound was prepared according to a similar procedure used for the synthesis of Compound 129. MS (m/z): [M+H]⁺−518.5, 520.1. H NMR (500 MHz, DMSO) δ 10.00 (s, 1H), 9.27 (s, 1H), 9.16 (s, 1H), 8.86 (s, 1H), 8.42 (s, 1H), 8.35 (br, 1H), 8.12 (d, J=5.7 Hz, 1H), 7.84 (d, J=8.8 Hz, 1H), 7.78 (br, 1H), 7.67 (d, J=10.8 Hz, 1H), 7.37 (br, 1H), 7.19 (d, J=7.9 Hz, 1H), 6.35 (d, J=5.7 Hz, 1H), 4.24 (t, J=8.5 Hz, 2H), 3.21-3.10 (m, 4H), 2.11 (s, 6H).

### Example 281: Preparation of N4-(6-chloroquinolin-3-yl)-N2-(6-((1s,3s)-3-(dimethylamino)cyclobutoxy)-5-methoxypyridin-3-yl)pyrimidine-2,4-diamine (Compound 131)

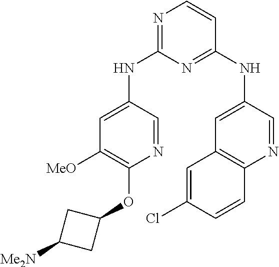 [SMILES: COc1cc(Nc2nccc(Nc3cnc4ccc(Cl)cc4c3)n2)cnc1O[C@H]1C[C@@H](N(C)C)C1]

The title compound was prepared according to the general SNAr procedure E. MS (m/z): [M+H]⁺492.6. ¹H NMR (400 MHz, DMSO) δ (ppm): 10.00 (br, 1H), 9.14 (s, 1H), 8.97 (s, 1H), 8.94-8.88 (m, 1H), 8.11 (d, J=5.7 Hz, 1H), 8.08 (d, J=2.2 Hz, 1H), 7.94 (d, J=8.9 Hz, 1H), 7.85 (s, 1H), 7.59 (dd, J=8.9, 2.4 Hz, 1H), 7.52 (br, 1H), 6.34 (d, J=5.7 Hz, 1H), 4.92 (dddd, J=7.3 Hz, 1H), 3.69 (s, 3H), 2.63-2.54 (m, 2H), 2.40-2.29 (m, 1H), 2.05 (s, 6H), 1.90-1.78 (m, 2H).

### Example 282: Preparation of N2-(6-((1s,3s)-3-(dimethylamino)cyclobutoxy)-5-methoxypyridin-3-yl)-N4-(quinolin-3-yl)pyrimidine-2,4-diamine (Compound 132)

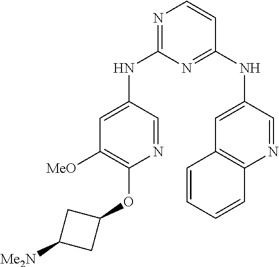 [SMILES: COc1cc(Nc2nccc(Nc3cnc4ccccc4c3)n2)cnc1O[C@H]1C[C@@H](N(C)C)C1]

The title compound was prepared according to the general SNAr procedure E. MS (m/z): [M+H]⁺458.4. ¹H NMR (400 MHz, DMSO) δ (ppm): 9.88 (s, 1H), 9.14 (s, 1H), 8.96-8.85 (m, 2H), 8.09 (d, J=5.7 Hz, 1H), 8.04 (d, J=2.1 Hz, 1H), 7.92 (d, J=8.3 Hz, 1H), 7.80-7.69 (m, 1H), 7.63-7.44 (m, 3H), 6.33 (d, J=5.7 Hz, 1H), 4.86 (dddd, J=7.6 Hz, 1H), 3.66 (s, 3H), 2.65-2.53 (m, 2H), 2.41-2.28 (m, 1H), 2.05 (s, 6H), 1.91-1.77 (m, 2H).

### Example 283: Preparation of N4-(3-bromo-8-fluoroquinolin-6-yl)-N2-(4-((1s,3s)-3-(dimethylamino)cyclobutoxy)-3-methoxyphenyl)pyrimidine-2,4-diamine (Compound 133)

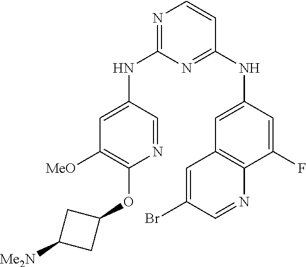 [SMILES: COc1cc(Nc2nccc(Nc3cc(F)c4ncc(Br)cc4c3)n2)cnc1O[C@H]1C[C@@H](N(C)C)C1]

The title compound was prepared according to the general SNAr procedure E with 3-bromo-8-fluoroquinolin-6-amine. MS (m/z): [M+H]⁺−553.6. ¹H NMR (500 MHz, DMSO) δ 9.90 (s, 1H), 9.08 (s, 1H), 8.81 (d, J=2.2 Hz, 1H), 8.48 (br, 1H), 8.32 (br, 1H), 8.10 (d, J=5.7 Hz, 1H), 7.91 (d, J=11.4 Hz, 1H), 7.31-7.20 (m, 2H), 6.78 (d, J=8.5 Hz, 1H), 6.28 (d, J=5.7 Hz, 1H), 4.36 (dddd, J=7.2 Hz, 1H), 3.68 (s, 3H), 2.61-2.54 (m, 2H), 2.36-2.28 (m, 1H), 2.05 (s, 6H), 1.88-1.79 (m, 2H).

### Example 284: Preparation of N4-(3-chloroquinoxalin-6-yl)-N2-(4-((1s,3s)-3-(dimethylamino)cyclobutoxy)-3-methoxyphenyl)pyrimidine-2,4-diamine (Compound 134)

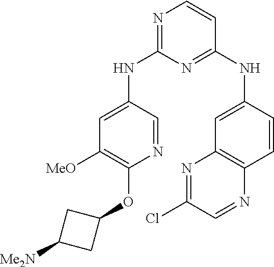 [SMILES: COc1cc(Nc2nccc(Nc3ccc4ncc(Cl)nc4c3)n2)cnc1O[C@H]1C[C@@H](N(C)C)C1]

The title compound was prepared according to the general SNAr procedure E with 3-chloroquinoxalin-6-amine. MS (m/z): [M+H]⁺−492.5. ¹H NMR (500 MHz, DMSO) δ 9.98 (br, 1H), 9.20 (s, 1H), 8.79 (s, 1H), 8.69 (s, 1H), 8.13 (d, J=5.6 Hz, 1H), 8.06-7.99 (m, 2H), 7.38 (dd, J=8.7, 2.4 Hz, 1H), 7.29 (br, 1H), 6.80 (d, J=8.7 Hz, 1H), 6.34 (d, J=5.7 Hz, 1H), 4.37 (dddd, J=7.2 Hz, 1H), 3.69 (s, 3H), 2.61-2.53 (m, 2H), 2.35-2.24 (m, 1H), 2.05 (s, 6H), 1.86-1.78 (m, 2H).

### Example 285: Preparation of Compound 135

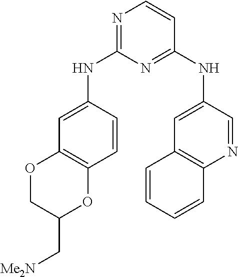 [SMILES: CN(C)CC1COc2cc(Nc3nccc(Nc4cnc5ccccc5c4)n3)ccc2O1]

### Step 1—Preparation of Intermediate 1-(4-bromo-2-(oxiran-2-ylmethoxy)phenyl)ethan-1-one

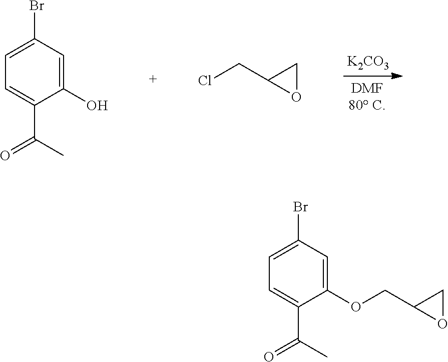

To a solution of 1-(4-bromo-2-hydroxyphenyl)ethan-1-one (1 equiv.) and K₂CO₃ (1.2 equiv.) in DMF (0.47 M) was added (+)-epichlorohydrine (5 equiv.). The reaction mixture was stirred at 80° C. for 2 hours. Upon cooling to room temperature, the mixture was added to water. Extraction was performed with MTBE. The organic layers were combined, washed with water, dried over anhydrous Na₂SO₄, and concentrated to dryness. The crude product was purified by flash chromatography (SiO₂, eluting with Hexanes/MTBE/CH₂Cl₂) to afford the title compound.

### Step 2—Preparation of intermediate (6-bromo-2,3-dihydrobenzo[b][1,4]dioxin-2-yl)methanol

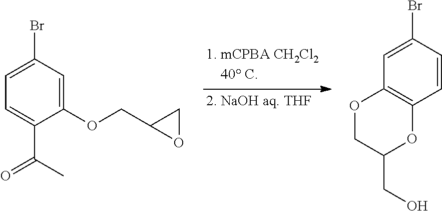

To a solution of 1-(4-bromo-2-(oxiran-2-ylmethoxy)phenyl)ethan-1-one (1 equiv.) in CH₂C₁₂ (0.19 M) was added mCPBA (4 equiv.). The reaction mixture was stirred at 40° C. for 20 hours. The reaction was cooled in an ice bath and quenched with a solution of aqueous sodium sulfite. The mixture was diluted with water and extracted with CH₂Cl₂. The organic extracts were combined and washed with NaHCO₃, aqueous sodium sulfite, dried over anhydrous Na₂SO₄, and concentrated to dryness to give the intermediate acetate ester.

The ester was diluted in THF (0.14 M) and NaOH (0.14 M, 1 M aqueous solution) was added. The reaction mixture was stirred for 2 hours. The mixture was diluted with water and extracted with EtOAc. The organic extracts were combined and washed with water, brine, dried over anhydrous Na₂SO₄, and concentrated to dryness to afford the title compound.

### Step 3—Preparation of Intermediate 1-(6-bromo-2,3-dihydrobenzo[b][1,4]dioxin-2-yl)-N,N-dimethylmethanamine

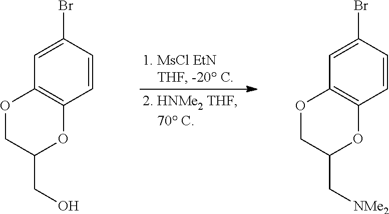 [SMILES: OCC1COc2cc(Br)ccc2O1 ; CN(C)CC1COc2cc(Br)ccc2O1]

To a solution of (6-bromo-2,3-dihydrobenzo[b][1,4]dioxin-2-yl)methanol (1 equiv.) in THF (0.25 M) at −20° C. was added trimethylamine (1.5 equiv.) and MsCl (1.3 equiv.). The reaction mixture was stirred at −20° C. for 30 mins. The mixture was filtered and concentrated to dryness to give the intermediate mesylate.

The mesylate was diluted in THF (0.4 M) in a pressure vessel and dimethylamine (20 equiv., 40% in water) was added. The reaction mixture was stirred at 70° C. for 16 hours. The mixture was concentrated to dryness and diluted with 1 M HCl aq. and washed with EtOAc. The aqueous acidic layer was basified to pH 10 using 6 NaOH, extracted with EtOAc, washed with brine, dried over anhydrous Na₂SO₄, and concentrated to dryness to afford the title compound. MS (m/z): [M+H]⁺−272.1, 274.1.

### Step 4—Preparation of Intermediate 2-((dimethylamino)methyl)-2,3-dihydrobenzo[b][1,4]dioxin-6-amine

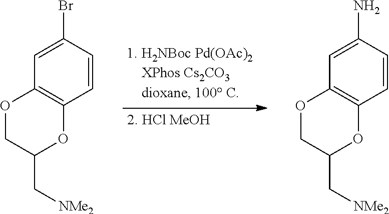 [SMILES: CN(C)CC1COc2cc(Br)ccc2O1 ; CN(C)CC1COc2cc(N)ccc2O1]

A mixture of 1-(6-bromo-2,3-dihydrobenzo[b][1,4]dioxin-2-yl)-N,N-dimethylmethanamine (1 equiv.), tert-butylcarbamate (1.2 equiv.), Pd(OAc)₂ (3 mol %), XPhos (9 mol %) and Cs₂CO₃ (2 equiv) in dioxane (0.2 M) was heated at a temperature 110° C. for 24 hours. The reaction was diluted with EtOAc/MeOH, filtered, and concentrated to dryness to give the boc-protected aniline intermediate. The aniline was diluted in MeOH (0.5 M) and concentrated HCl (0.5 M). The reaction mixture was stirred for 30 mins. The mixture was diluted with water and washed with MTBE. The aqueous layer was basified to pH 10 with 6 M NaOH, extracted with EtOAc, washed with brine, dried over anhydrous Na₂SO₄, and concentrated to dryness. The crude product was purified by flash chromatography (SiO₂, eluting with EtOAc/EtOH/NH₄OH) to afford the title compound. MS (m/z): [M+H]⁺−209.2.

### Step 5—Preparation of N2-(2-((dimethylamino)methyl)-2,3-dihydrobenzo[b][1,4]dioxin-6-yl)-N4-(quinolin-3-yl)pyrimidine-2,4-diamine

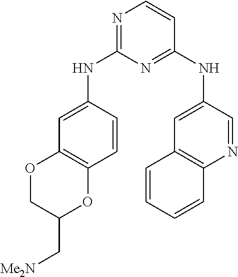 [SMILES: CN(C)CC1COc2cc(Nc3nccc(Nc4cnc5ccccc5c4)n3)ccc2O1]

The title compound was prepared according to the general SNAr procedure E. MS (m/z): [M+H]⁺429.4. ¹H NMR (500 MHz, DMSO) δ 9.85 (br, 1H), 9.09 (s, 1H), 8.98 (br, 1H), 8.92 (d, J=2.4 Hz, 1H), 8.08 (d, J=5.7 Hz, 1H), 7.93 (d, J=8.4 Hz, 1H), 7.82 (d, J=6.6 Hz, 1H), 7.62-7.53 (m, 2H), 7.37 (s, 1H), 7.12-7.08 (m, 1H), 6.78 (d, J=8.7 Hz, 1H), 6.32-6.29 (m, 1H), 4.30-4.23 (m, 2H), 3.94 (dd, J=11.7, 7.6 Hz, 1H), 2.53-2.52 (m, 2H), 2.24 (s, 6H).

### Example 286: Preparation of N4-(6-chloroquinolin-3-yl)-N2-(2-((dimethylamino)methyl)-2,3-dihydrobenzo[b][1,4]dioxin-6-yl)pyrimidine-2,4-diamine (Compound 136)

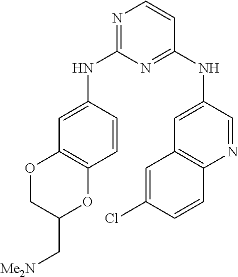 [SMILES: CN(C)CC1COc2cc(Nc3nccc(Nc4cnc5ccc(Cl)cc5c4)n3)ccc2O1]

The title compound was prepared according to a similar procedure used for the synthesis of Compound 135. MS (m/z): [M+H]⁺−463.5. ¹H NMR (500 MHz, DMSO) δ (ppm): 9.93 (s, 1H), 9.10 (s, 1H), 9.03 (br, 1H), 8.94-8.85 (m, 1H), 8.10 (d, J=5.7 Hz, 1H), 7.94 (d, J=8.9 Hz, 1H), 7.89 (br, 1H), 7.59 (dd, J=8.9, 2.4 Hz, 1H), 7.43-7.38 (m, 1H), 7.07-7.01 (m, 1H), 6.81 (d, J=8.7 Hz, 1H), 6.35-6.29 (m, 1H), 4.34-4.25 (m, 2H), 3.99-3.91 (m, 1H), 2.53 (s, 2H), 2.24 (s, 6H).

### Example 287: Preparation of Compound 137

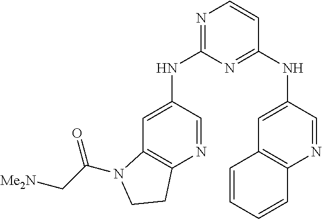 [SMILES: CN(C)CC(=O)N1CCc2ncc(Nc3nccc(Nc4cnc5ccccc5c4)n3)cc21]

### Step 1—Preparation of Intermediate 1-(6-amino-2,3-dihydro-1H-pyrrolo[3,2-b]255yridine-1-yl)-2-(dimethylamino)ethan-1-one

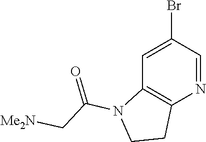 [SMILES: CN(C)CC(=O)N1CCc2ncc(Br)cc21]

The title compound was prepared according to the procedures J and K starting from 6-bromo-2,3-dihydro-1H-pyrrolo[3,2-b]pyridine. MS (m/z): [M+H]⁺−284.2, 286.0.

### Step 2—Preparation of intermediate tert-butyl (1-(dimethylglycyl)-2,3-dihydro-1H-pyrrolo[3,2-b]pyridin-6-yl)carbamate

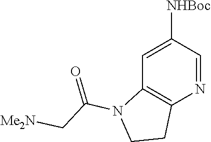 [SMILES: CN(C)CC(=O)N1CCc2ncc(NC(=O)OC(C)(C)C)cc21]

The title compound was prepared according to the procedure L. MS (m/z): [M+H]⁺−321.3.

### Step 3—Preparation of Intermediate 1-(6-amino-2,3-dihydro-1H-pyrrolo[3,2-b]pyridin-1-yl)-2-(dimethylamino)ethan-1-one

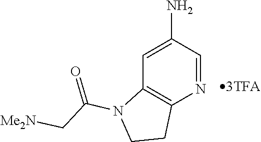 [SMILES: CN(C)CC(=O)N1CCc2ncc(N)cc21]

The title compound was prepared according to the procedure M. MS (m/z): [M+H]⁺−221.1.

### Step 4—Preparation of 2-(dimethylamino)-1-(6-((4-(quinolin-3-ylamino)pyrimidin-2-yl)amino)-2,3-dihydro-1H-pyrrolo[3,2-b]pyridin-1-yl)ethan-1-one

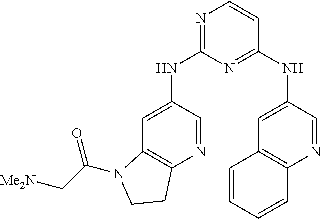 [SMILES: CN(C)CC(=O)N1CCc2ncc(Nc3nccc(Nc4cnc5ccccc5c4)n3)cc21]

The title compound was prepared using general procedure F. MS (m/z): [M+H]⁺−441.4. ¹H NMR (500 MHz, DMSO) δ 9.96 (s, 1H), 9.44 (s, 1H), 9.06 (s, 1H), 8.95 (s, 1H), 8.66 (s, 1H), 8.58 (s, 1H), 8.17 (d, J=5.7 Hz, 1H), 8.00-7.89 (m, 1H), 7.77 (br, 1H), 7.64-7.59 (m, 2H), 6.43-6.40 (m, 1H), 4.28 (t, J=8.6 Hz, 2H), 3.25 (t, J=8.5 Hz, 2H), 3.18 (s, 2H), 2.20 (s, 6H).

### Example 288: Preparation of 1-(6-((4-((7,8-difluoroquinolin-3-yl)amino)pyrimidin-2-yl)amino)indolin-1-yl)-2-(dimethylamino)ethan-1-one (Compound 138)

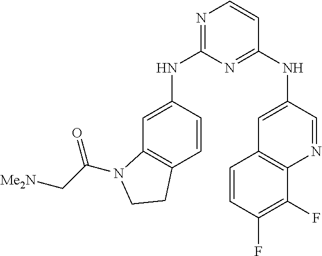 [SMILES: CN(C)CC(=O)N1CCc2ccc(Nc3nccc(Nc4cnc5c(F)c(F)ccc5c4)n3)cc21]

The title compound was prepared according to a similar procedure used for the synthesis of Compound 129. MS (m/z): [M+H]⁺−476.3. ¹H NMR (500 MHz, DMSO) δ 10.02 (s, 1H), 9.28 (s, 1H), 9.18 (s, 1H), 8.99 (s, 1H), 8.37 (s, 1H), 8.11 (d, J=5.7 Hz, 1H), 7.77-7.66 (m, 1H), 7.50 (s, 1H), 7.41 (s, 1H), 7.15 (d, J=8.1 Hz, 1H), 6.34 (d, J=5.6 Hz, 1H), 4.18 (t, J=8.2 Hz, 2H), 3.18-3.06 (m, 4H), 2.13 (s, 6H).

### Example 289: Preparation of Compound 139

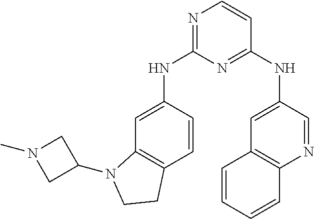 [SMILES: CN1CC(N2CCc3ccc(Nc4nccc(Nc5cnc6ccccc6c5)n4)cc32)C1]

### Step 1—Preparation of Intermediate 1-(1-methylazetidin-3-yl)-6-nitroindoline

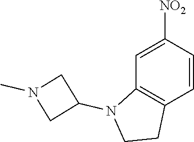 [SMILES: CN1CC(N2CCc3ccc([N+](=O)[O-])cc32)C1]

The title compound was prepared according to the procedure N starting from 6-nitroindoline. MS (m/z): [M+H]⁺−234.2.

### Step 2—Preparation of Intermediate 1-(1-methylazetidin-3-yl)indolin-6-amine

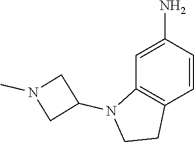

The title compound was prepared according to the general procedure H. MS (m/z): [M+H]⁺−204.3.

### Step 3—Preparation of N2-(1-(1-methylazetidin-3-yl)indolin-6-yl)-N4-(quinolin-3-yl)pyrimidine-2,4-diamine

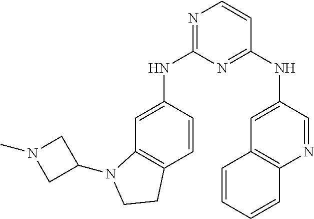 [SMILES: CN1CC(N2CCc3ccc(Nc4nccc(Nc5cnc6ccccc6c5)n4)cc32)C1]

The title compound was prepared according to the general SNAr procedure E. MS (m/z): [M+H]⁺424.5. ¹H NMR (500 MHz, DMSO) δ 9.85 (s, 1H), 9.10-9.01 (m, 2H), 9.01-8.94 (m, 1H), 8.13 (d, J=5.7 Hz, 1H), 7.97 (d, J=7.8 Hz, 1H), 7.84 (d, J=6.9 Hz, 1H), 7.65-7.57 (m, 2H), 7.07-7.01 (m, 1H), 6.98 (d, J=7.8 Hz, 1H), 6.82 (s, 1H), 6.34 (d, J=5.7 Hz, 1H), 3.78-3.71 (m, 1H), 3.44-3.39 (m, 2H), 3.38-3.32 (m, 2H), 2.99-2.94 (m, 2H), 2.90 (t, J=8.0 Hz, 2H), 2.19 (s, 3H).

### Preparation of Example 290: Preparation of Compound 140

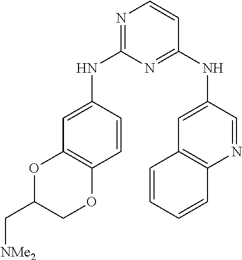 [SMILES: CN(C)CC1COc2ccc(Nc3nccc(Nc4cnc5ccccc5c4)n3)cc2O1]

### Step 1—Preparation of Intermediate 7-bromo-2-(iodomethyl)-2,3-dihydrobenzo[b][1,4]dioxine

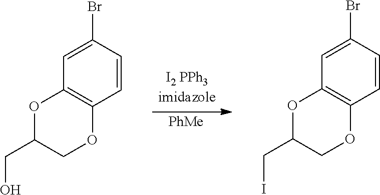 [SMILES: OCC1COc2ccc(Br)cc2O1 ; "PhMe" ; Brc1ccc2c(c1)OC(CI)CO2]

To a solution of (7-bromo-2,3-dihydrobenzo[b][1,4]dioxin-2-yl)methanol (1 equiv.) in PhMe (0.13 M) was added imidazole (1.1 equiv.), triphenylphospine (1.05 equiv.), and iodine (1.1 equiv.). The reaction mixture was stirred for 2 hours. The mixture was filtered and concentrated to dryness. The crude product was purified by flash chromatography (SiO₂, eluting with EtOAc/Hexanes) to afford the title compound.

### Step 2—Preparation of Intermediate 1-(7-bromo-2,3-dihydrobenzo[b][1,4]dioxin-2-yl)-N,N-dimethylmethanamine

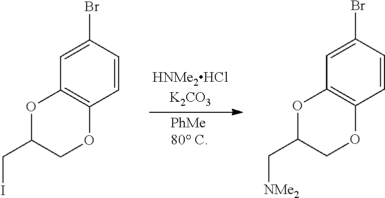 [SMILES: Brc1ccc2c(c1)OC(CI)CO2 ; "PhMe80° C." ; CN(C)CC1COc2ccc(Br)cc2O1]

To a solution of 7-bromo-2-(iodomethyl)-2,3-dihydrobenzo[b][1,4]dioxine (1 equiv.) in PhMe (0.46 M) was added dimethylamine hydrochloride (3 equiv.), and K₂CO₃ (2.6 equiv.). The reaction mixture was stirred at 80° C. for 16 hours. The mixture was concentrated to dryness. The crude product was purified by flash chromatography (SiO₂, eluting with MeOH/CH₂Cl₂) to afford the title compound. MS (m/z): [M+H]⁺−272.1, 274.0.

### Step 3—Preparation of intermediate tert-butyl (3-((dimethylamino)methyl)-2,3-dihydrobenzo[b][1,4]dioxin-6-yl)carbamate

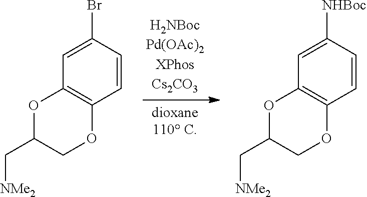 [SMILES: CN(C)CC1COc2ccc(Br)cc2O1 ; CN(C)CC1COc2ccc(NC(=O)OC(C)(C)C)cc2O1]

A mixture of 1-(7-bromo-2,3-dihydrobenzo[b][1,4]dioxin-2-yl)-N,N-dimethylmethanamine (1 equiv.), tert-butylcarbamate (1.2 equiv.), Pd(OAc)₂ (3 mol %), XPhos (9 mol %) and Cs₂CO₃ (2 equiv) in Dioxane (0.2 M) was heated at a temperature 110° C. for 24 hours. The reaction was diluted with EtOAc/MeOH, filtered, and concentrated to dryness. The crude product was purified by flash chromatography (SiO₂, eluting with MeOH/CH₂Cl₂/NH₄OH) to afford the title compound. MS (m/z): [M+H]⁺−309.3.

### Step 4—Preparation of Intermediate 3-((dimethylamino)methyl)-2,3-dihydrobenzo[b][1,4]dioxin-6-amine bis(2,2,2-trifluoroacetate)

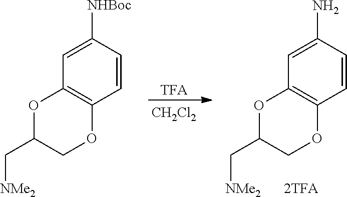

To a solution of tert-butyl (3-((dimethylamino)methyl)-2,3-dihydrobenzo[b][1,4]dioxin-6-yl)carbamate (1 equiv.) in CH₂Cl₂ (0.05 M) was added TFA (20 equiv.). The reaction mixture was stirred for 2 hours. The mixture was concentrated to dryness to afford the title compound. MS (m/z): [M+H]⁺−209.2.

### Step 5—Preparation of N2-(3-((dimethylamino)methyl)-2,3-dihydrobenzo[b][1,4]dioxin-6-yl)-N4-(quinolin-3-yl)pyrimidine-2,4-diamine

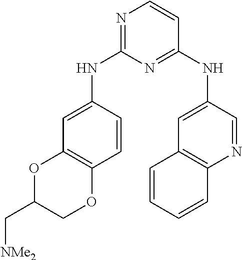 [SMILES: CN(C)CC1COc2ccc(Nc3nccc(Nc4cnc5ccccc5c4)n3)cc2O1]

The title compound was prepared according to the general SNAr procedure E. MS (m/z): [M+H]⁺429.5. H NMR (500 MHz, DMSO) δ 9.83 (s, 1H), 9.11 (s, 1H), 8.99 (s, 1H), 8.95-8.91 (m, 1H), 8.10 (d, J=5.7 Hz, 1H), 7.94 (d, J=7.4 Hz, 1H), 7.83 (br, 1H), 7.64-7.52 (m, 2H), 7.39 (s, 1H), 7.13-7.05 (m, 1H), 6.79 (d, J=8.7 Hz, 1H), 6.32 (d, J=5.7 Hz, 1H), 4.34-4.24 (m, 2H), 3.97-3.87 (m, 1H), 2.48-2.41 (m, 2H), 2.19 (s, 6H).

### Example 291: Preparation of 1-(6-((4-((6-chloroquinolin-3-yl)amino)pyrimidin-2-yl)amino)-2,3-dihydro-1H-pyrrolo[3,2-b]pyridin-1-yl)-2-(dimethylamino)ethan-1-one (Compound 141)

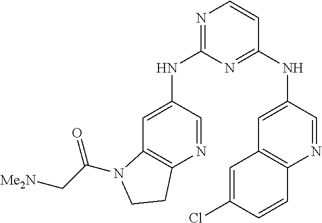 [SMILES: CN(C)CC(=O)N1CCc2ncc(Nc3nccc(Nc4cnc5ccc(Cl)cc5c4)n3)cc21]

The title compound was prepared according to a similar procedure used for the synthesis of Compound 137. MS (m/z): [M+H]⁺−475.4. ¹H NMR (500 MHz, DMSO) δ 10.06 (s, 1H), 9.41 (s, 1H), 9.09 (s, 1H), 8.90 (s, 1H), 8.62 (s, 1H), 8.53 (s, 1H), 8.17 (d, J=5.7 Hz, 1H), 7.93 (d, J=8.9 Hz, 1H), 7.78 (s, 1H), 7.59 (dd, J=8.9, 2.4 Hz, 1H), 6.41 (d, J=5.7 Hz, 1H), 4.29 (t, J=8.6 Hz, 2H), 3.25 (t, J=8.6 Hz, 2H), 3.19 (s, 2H), 2.16 (s, 6H).

### Example 292: Preparation of N4-(6-chloroquinolin-3-yl)-N2-(3-((dimethylamino)methyl)-2,3-dihydrobenzo[b][1,4]dioxin-6-yl)pyrimidine-2,4-diamine (Compound 142)

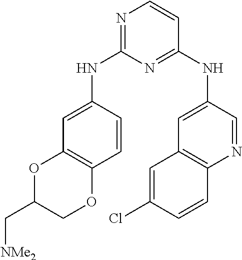 [SMILES: CN(C)CC1COc2ccc(Nc3nccc(Nc4cnc5ccc(Cl)cc5c4)n3)cc2O1]

The title compound was prepared according to a similar procedure used for the synthesis of Compound 140. MS (m/z): [M+H]⁺−463.5. ¹H NMR (500 MHz, DMSO) δ 9.94 (s, 1H), 9.10 (s, 1H), 9.03 (s, 1H), 8.92 (s, 1H), 8.11 (d, J=5.7 Hz, 1H), 7.95 (d, J=8.9 Hz, 1H), 7.85 (s, 1H), 7.59 (dd, J=8.9, 2.4 Hz, 1H), 7.41 (s, 1H), 7.06-6.99 (m, 1H), 6.81 (d, J=8.7 Hz, 1H), 6.32 (d, J=5.7 Hz, 1H), 4.33-4.25 (m, 2H), 3.99-3.90 (m, 1H), 2.48-2.38 (m, 2H), 2.18 (s, 6H).

### Example 293: Preparation of Compound 143

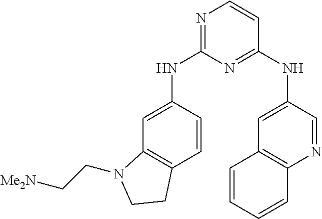 [SMILES: CN(C)CCN1CCc2ccc(Nc3nccc(Nc4cnc5ccccc5c4)n3)cc21]

### Step 1—Preparation of Intermediate 1-(2-(dimethylamino)ethyl)indolin-6-amine

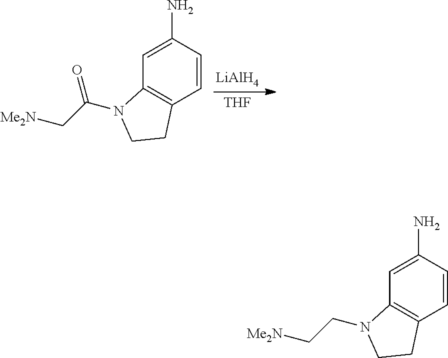

To a solution of 1-(6-aminoindolin-1-yl)-2-(dimethylamino)ethan-1-one (1 equiv.) in THF (0.2 M) was added LiAlH₄ (3 equiv.). Upon completion the reaction mixture was diluted with NaOH 1M aq. and EtOAc, and then filtered. The filtrate was extracted with EtOAc, dried over anhydrous Na₂SO₄, and concentrated to dryness to afford the title compound. MS (m/z): [M+H]⁺−206.3.

### Step 2—Preparation of N2-(1-(2-(dimethylamino)ethyl)indolin-6-yl)-N4-(quinolin-3-yl)pyrimidine-2,4-diamine

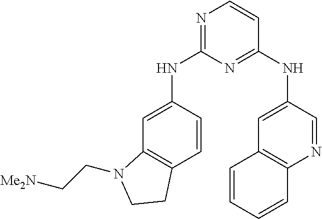 [SMILES: CN(C)CCN1CCc2ccc(Nc3nccc(Nc4cnc5ccccc5c4)n3)cc21]

The title compound was prepared according to the general SNAr procedure E. MS (m/z): [M+H]⁺426.4. ¹H NMR (500 MHz, DMSO) δ 9.81 (s, 1H), 9.05-8.98 (m, 2H), 8.92 (d, J=2.6 Hz, 1H), 8.08 (d, J=5.7 Hz, 1H), 7.91 (d, J=7.4 Hz, 1H), 7.77 (d, J=6.3 Hz, 1H), 7.62-7.52 (m, 2H), 6.95-6.90 (m, 2H), 6.87 (s, 1H), 6.29 (d, J=5.7 Hz, 1H), 3.35 (t, J=8.3 Hz, 2H), 2.97 (t, J=7.0 Hz, 2H), 2.87 (t, J=8.3 Hz, 2H), 2.30 (t, J=6.9 Hz, 2H), 2.06 (s, 6H).

### Example 294: Preparation of (S)—N4-(6-chloroquinolin-3-yl)-N2-(2-((dimethylamino)methyl)-2,3-dihydrobenzo[b][1,4]dioxin-6-yl)pyrimidine-2,4-diamine (Compound 144)

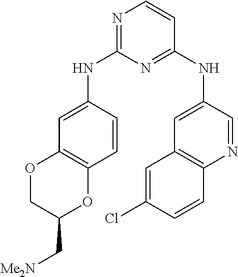 [SMILES: CN(C)C[C@H]1COc2cc(Nc3nccc(Nc4cnc5ccc(Cl)cc5c4)n3)ccc2O1]

The title compound was prepared according to a similar procedure used for the synthesis of Compound 135 and then enantiomerically resolved by chiral-HPLC. MS (m/z): [M+H]⁺−463.5. ¹H NMR (400 MHz, DMSO) δ 9.95 (s, 1H), 9.11 (s, 1H), 9.05 (s, 1H), 8.94-8.89 (m, 1H), 8.11 (d, J=5.7 Hz, 1H), 7.95 (d, J=8.9 Hz, 1H), 7.91 (s, 1H), 7.60 (dd, J=8.9, 2.4 Hz, 1H), 7.42 (s, 1H), 7.05 (d, J=6.8 Hz, 1H), 6.82 (d, J=8.7 Hz, 1H), 6.33 (d, J=5.7 Hz, 1H), 4.35-4.26 (m, 2H), 4.01-3.92 (m, 1H), 2.55-2.52 (m, 2H), 2.25 (s, 6H).

### Example 295: Preparation of (R)—N4-(6-chloroquinolin-3-yl)-N2-(2-((dimethylamino)methyl)-2,3-dihydrobenzo[b][1,4]dioxin-6-yl)pyrimidine-2,4-diamine (Compound 145)

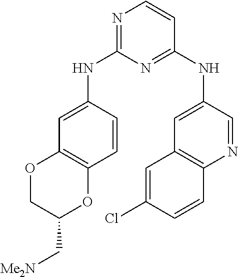 [SMILES: CN(C)C[C@@H]1COc2cc(Nc3nccc(Nc4cnc5ccc(Cl)cc5c4)n3)ccc2O1]

The title compound was prepared according to a similar procedure used for the synthesis of Compound 135 and then enantiomerically resolved by chiral-HPLC. MS (m/z): [M+H]⁺−463.5. ¹H NMR (400 MHz, DMSO) 1H NMR (400 MHz, DMSO) δ 9.95 (s, 1H), 9.11 (s, 1H), 9.05 (s, 1H), 8.94-8.89 (m, 1H), 8.11 (d, J=5.7 Hz, 1H), 7.95 (d, J=8.9 Hz, 1H), 7.91 (s, 1H), 7.60 (dd, J=8.9, 2.4 Hz, 1H), 7.42 (s, 1H), 7.05 (d, J=6.8 Hz, 1H), 6.82 (d, J=8.7 Hz, 1H), 6.33 (d, J=5.7 Hz, 1H), 4.35-4.26 (m, 2H), 4.01-3.92 (m, 1H), 2.55-2.52 (m, 2H), 2.25 (s, 6H).

### Example 296: Preparation of 1-(5-((4-((6-chloroquinolin-3-yl)amino)pyrimidin-2-yl)amino)indolin-1-yl)-2-(dimethylamino)ethan-1-one (Compound 146)

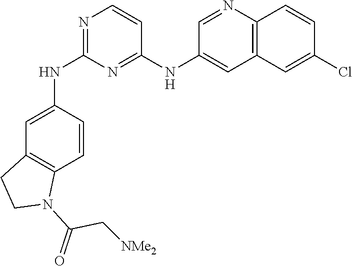 [SMILES: CN(C)CC(=O)N1CCc2cc(Nc3nccc(Nc4cnc5ccc(Cl)cc5c4)n3)ccc21]

The title compound was prepared according the procedures J, K, H, and E starting from 5-nitroindoline. MS (m/z): [M+H]⁺−474.5. ¹H NMR (400 MHz, DMSO) δ 9.96 (s, 1H), 9.20 (s, 1H), 9.02-8.91 (m, 2H), 8.12 (d, J=5.6 Hz, 1H), 8.01 (d, J=8.6 Hz, 1H), 7.94 (d, J=8.9 Hz, 1H), 7.82 (s, 1H), 7.67 (s, 1H), 7.58 (dd, J=8.9, 2.3 Hz, 1H), 7.37 (d, J=8.3 Hz, 1H), 6.32 (d, J=5.7 Hz, 1H), 4.16 (t, J=8.5 Hz, 2H), 3.20 (s, 2H), 3.06 (t, J=8.2 Hz, 2H), 2.28 (s, 6H).

### Example 297: Preparation of 1-(6-((4-((6,7-difluoroquinolin-3-yl)amino)pyrimidin-2-yl)amino)indolin-1-yl)-2-(dimethylamino)ethan-1-one (Compound 147)

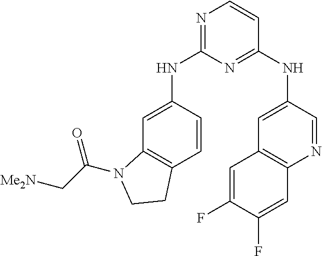 [SMILES: CN(C)CC(=O)N1CCc2ccc(Nc3nccc(Nc4cnc5cc(F)c(F)cc5c4)n3)cc21]

The title compound was prepared according to the procedures used to synthesize Compound 129. MS (m/z): [M+H]⁺−476.3. ¹H NMR (500 MHz, DMSO) δ 9.98 (s, 1H), 9.27 (s, 1H), 9.17 (s, 1H), 8.97 (s, 1H), 8.40 (s, 1H), 8.15 (d, J=5.7 Hz, 1H), 7.99-7.91 (m, 1H), 7.64 (br, 1H), 7.49 (br, 1H), 7.21 (d, J=8.1 Hz, 1H), 6.37 (d, J=5.7 Hz, 1H), 4.23 (t, J=8.3 Hz, 2H), 3.22-3.13 (m, 4H), 2.20 (s, 6H).

### Example 298: Preparation of Compound 148

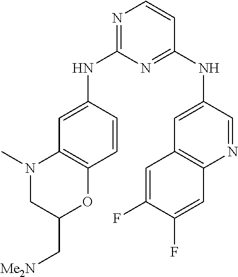 [SMILES: CN(C)CC1CN(C)c2cc(Nc3nccc(Nc4cnc5cc(F)c(F)cc5c4)n3)ccc2O1]

### Step 1—Preparation of intermediate (6-bromo-3,4-dihydro-2H-benzo[b][1,4]oxazin-2-yl)methanol

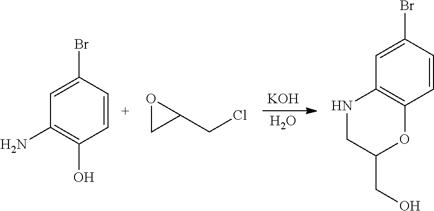

To a solution KOH (1.4 equiv.) in H₂O (0.2 M) was added 2-amino-4-bromophenol (1 equiv.). The mixture was stirred for 15 mins and then (+)-epichlorohydrine (1.2 equiv.) was added. The reaction mixture was stirred for 30 mins. The mixture neutralized with 1M HCl and then extracted with EtOAc. The combined organics was washed with brine, dried with Na₂SO₄, and concentrated to dryness. The crude product was purified by flash chromatography (SiO₂, eluting with EtOAc/Hexanes) to afford the title compound.

### Step 2—Preparation of intermediate (6-bromo-4-methyl-3,4-dihydro-2H-benzo[b][1,4]oxazin-2-yl)methanol

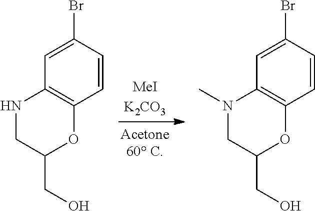

To a solution (6-bromo-3,4-dihydro-2H-benzo[b][1,4]oxazin-2-yl)methanol (1.4 equiv.) in acetone (0.2 M) was added iodomethane (2.2 equiv.) and K₂CO₃ (1.5 equiv.). The reaction mixture was stirred at 60° C. for 60 hours. The mixture was filtered and concentrated to dryness. The crude product was purified by flash chromatography (SiO₂, eluting with EtOAc/Hexanes) to afford the title compound.

### Step 3—Preparation of Intermediate 1-(6-bromo-4-methyl-3,4-dihydro-2H-benzo[b][1,4]oxazin-2-yl)-N,N-dimethylmethanamine

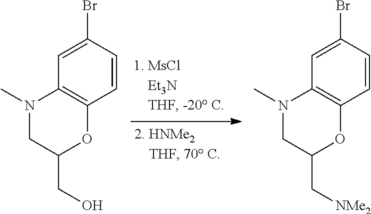 [SMILES: CN1CC(CO)Oc2ccc(Br)cc21 ; CN(C)CC1CN(C)c2cc(Br)ccc2O1]

To a solution of (6-bromo-4-methyl-3,4-dihydro-2H-benzo[b][1,4]oxazin-2-yl)methanol (1 equiv.) in THF (0.15 M) at 0° C. was added trimethylamine (1.5 equiv.) and MsCl (1.3 equiv.). The reaction mixture was stirred at 0° C. for 30 mins. The mixture was filtered and concentrated to dryness to give the intermediate mesylate.

The mesylate was diluted in THF (0.4 M) in a pressure vessel and dimethylamine (16 equiv., 40% in water) was added. The reaction mixture was stirred at 70° C. for 16 hours. The mixture was concentrated to dryness and diluted with 1 M HCl aq. and washed with EtOAc. The aqueous acidic layer was basified to pH 10 using 6 NaOH, extracted with EtOAc, washed with brine, dried over anhydrous Na₂SO₄, and concentrated to dryness to afford the title compound. MS (m/z): [M+H]⁺−285.1, 287.1.

### Step 4—Preparation of intermediate tert-butyl (2-((dimethylamino)methyl)-4-methyl-3,4-dihydro-2H-benzo[b][1,4]oxazin-6-yl)carbamate

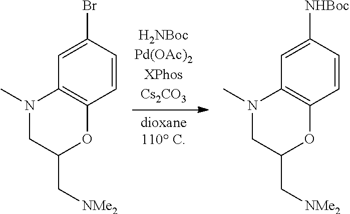 [SMILES: CN(C)CC1CN(C)c2cc(Br)ccc2O1 ; CN(C)CC1CN(C)c2cc(NC(=O)OC(C)(C)C)ccc2O1]

A mixture of 1-(6-bromo-4-methyl-3,4-dihydro-2H-benzo[b][1,4]oxazin-2-yl)-N,N-dimethylmethanamine (1 equiv.), tert-butylcarbamate (1.2 equiv.), Pd(OAc)₂ (5 mol %), XPhos (15 mol %) and Cs₂CO₃ (2 equiv) in Dioxane (0.2 M) was purged with N₂. The reaction mixture was heated at a temperature 100° C. for 16 hours. The reaction was diluted with EtOAc/MeOH, filtered, and concentrated to dryness. The crude product was purified by flash chromatography (SiO₂, eluting with MeOH/CH₂Cl₂/NH₄OH) to afford the title compound. MS (m/z): [M+H]⁺−322.3.

### Step 4—Preparation of Intermediate 2-((dimethylamino)methyl)-4-methyl-3,4-dihydro-2H-benzo[b][1,4]oxazin-6-amine tris(2,2,2-trifluoroacetate)

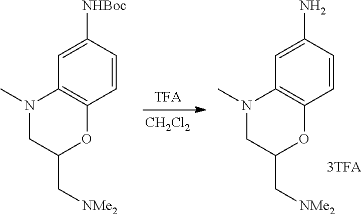

To a solution of tert-butyl (2-((dimethylamino)methyl)-4-methyl-3,4-dihydro-2H-benzo[b][1,4]oxazin-6-yl)carbamate (1 equiv.) in CH₂Cl₂ (0.05 M) was added TFA (20 equiv.). The reaction mixture was stirred for 5 hours. The mixture was concentrated to dryness to afford the title compound. MS (m/z): [M+H]⁺−222.2.

### Step 5—Preparation of N4-(6,7-difluoroquinolin-3-yl)-N2-(2-((dimethylamino)methyl)-4-methyl-3,4-dihydro-2H-benzo[b][1,4]oxazin-6-yl)pyrimidine-2,4-diamine

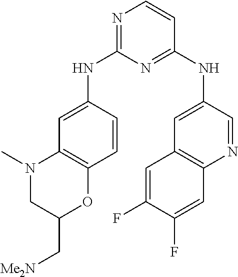 [SMILES: CN(C)CC1CN(C)c2cc(Nc3nccc(Nc4cnc5cc(F)c(F)cc5c4)n3)ccc2O1]

The title compound was prepared according to the general SNAr procedure E. MS (m/z): [M+H]⁺478.4. ¹H NMR (500 MHz, DMSO) δ (ppm): 9.88 (s, 1H), 9.04 (br, 1H), 8.93-8.86 (m, 2H), 8.08 (d, J=5.6 Hz, 1H), 7.96-7.87 (m, 1H), 7.67 (br, 1H), 6.98 (s, 1H), 6.85 (s, 1H), 6.65 (d, J=8.4 Hz, 1H), 6.27 (d, J=5.7 Hz, 1H), 4.34-4.26 (m, 1H), 3.32-3.26 (m, 1H), 2.98 (dd, J=11.7, 7.9 Hz, 1H), 2.67 (s, 3H), 2.50-2.46 (m, 2H), 2.24 (s, 6H).

### Example 299: Preparation of 1-(6-((4-((6,7-difluoroquinolin-3-yl)amino)pyrimidin-2-yl)amino)-2,3-dihydro-4H-benzo[b][1.4]oxazin-4-yl)-2-(dimethylamino)ethan-1-one (Compound 149)

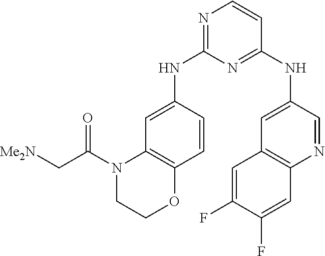 [SMILES: CN(C)CC(=O)N1CCOc2ccc(Nc3nccc(Nc4cnc5cc(F)c(F)cc5c4)n3)cc21]

The title compound was prepared according to the procedures used to synthesize Compound 137 starting from 6-bromo-3,4-dihydro-2H-benzo[b][1,4]oxazine. MS (m/z): [M+H]⁺−492.4. ¹H NMR (500 MHz, DMSO) δ 9.89 (s, 1H), 9.09 (s, 1H), 9.00-8.90 (m, 2H), 8.17 (br, 1H), 8.08 (d, J=5.7 Hz, 1H), 7.93 (dd, J=9.8 Hz, 1H), 7.66 (br, 1H), 7.34 (s, 1H), 6.85 (d, J=8.8 Hz, 1H), 6.29 (d, J=5.7 Hz, 1H), 4.27 (t, J=4.4 Hz, 2H), 3.93 (t, J=4.4 Hz, 2H), 3.19 (s, 2H), 2.11 (s, 6H).

### Example 300: Preparation of 1 N4-(6,7-difluoroquinolin-3-yl)-N2-(4-methyl-3,4-dihydro-2H-benzo[b][1,4]oxazin-6-yl)pyrimidine-2,4-diamine (Compound 150)

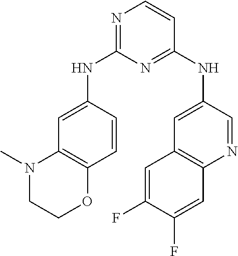 [SMILES: CN1CCOc2ccc(Nc3nccc(Nc4cnc5cc(F)c(F)cc5c4)n3)cc21]

The title compound was prepared according to the general SNAr procedure E with 4-methyl-3,4-dihydro-2H-benzo[b][1,4]oxazin-6-amine. MS (m/z): [M+H]⁺−421.4. ¹H NMR (500 MHz, DMSO) δ 9.87 (s, 1H), 9.04 (s, 1H), 8.94-8.85 (m, 2H), 8.07 (d, J=5.6 Hz, 1H), 7.92 (dd, J=11.4, 8.2 Hz, 1H), 7.69 (br, 1H), 6.98 (s, 1H), 6.85 (s, 1H), 6.62 (d, J=8.4 Hz, 1H), 6.26 (d, J=5.7 Hz, 1H), 4.27-4.22 (m, 2H), 3.25-3.20 (m, 2H), 2.69 (s, 3H).

### Example 301: Preparation of 3-((2-((1-(dimethylglycyl)indolin-5-yl)amino)pyrimidin-4-yl)amino)-8-fluoroquinolin-2(1H)-one (Compound 151)

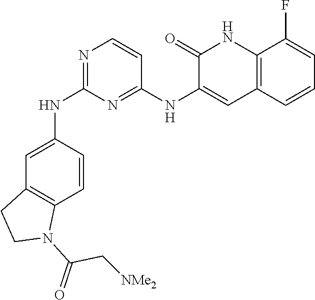 [SMILES: CN(C)CC(=O)N1CCc2cc(Nc3nccc(Nc4cc5cccc(F)c5[nH]c4=O)n3)ccc21]

The title compound was prepared according to a similar procedure used for the synthesis of Compound 129. MS (m/z): [M+H]⁺−474.4. ¹H NMR (500 MHz, DMSO) δ 9.23 (s, 1H), 9.09 (s, 1H), 9.02 (br, 1H), 8.11 (d, J=5.7 Hz, 1H), 8.06 (d, J=8.8 Hz, 1H), 7.64 (s, 1H), 7.42 (d, J=8.2 Hz, 1H), 7.35-7.22 (m, 2H), 7.19-7.11 (m, 1H), 6.79 (d, J=5.7 Hz, 1H), 4.18 (t, J=8.5 Hz, 2H), 3.23 (s, 2H), 3.09 (t, J=8.4 Hz, 2H), 2.31 (s, 6H).

### Example 302: Preparation of 1-(7-((4-((6,7-difluoroquinolin-3-yl)amino)pyrimidin-2-yl)amino)-2,3-dihydro-4H-benzo[b][1.4]oxazin-4-yl)-2-(dimethylamino)ethan-1-one (Compound 152)

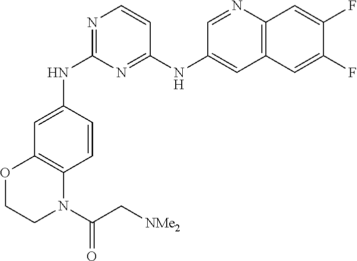 [SMILES: CN(C)CC(=O)N1CCOc2cc(Nc3nccc(Nc4cnc5cc(F)c(F)cc5c4)n3)ccc21]

The title compound was prepared according to general procedures J, K, M, and E starting from 7-nitro-3,4-dihydro-2H-benzo[b][1,4]oxazine. MS (m/z): [M+H]⁺−492.5. ¹H NMR (500 MHz, DMSO) δ 9.96 (s, 1H), 9.25 (s, 1H), 9.02 (s, 1H), 8.94 (s, 1H), 8.26 (s, 1H), 8.13 (d, J=5.7 Hz, 1H), 7.94 (dd, J=11.5, 8.1 Hz, 1H), 7.86-7.76 (m, 1H), 7.46 (s, 1H), 7.11 (d, J=10.2 Hz, 1H), 6.35 (d, J=5.7 Hz, 1H), 4.28-4.19 (m, 2H), 3.92 (s, 2H), 3.47-3.30 (m, 2H), 2.24 (s, 6H).

### Example 303: Preparation of N2-(2-chloro-4-((1s,3s)-3-(dimethylamino)cyclobutoxy)-5-methoxyphenyl)-N4-(6,7-difluoroquinolin-3-yl)pyrimidine-2,4-diamine (Compound 153)

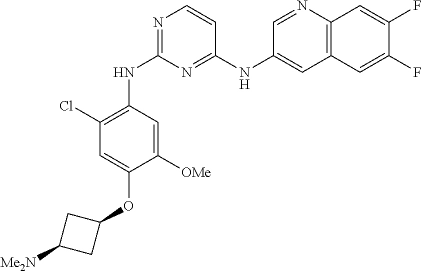 [SMILES: COc1cc(Nc2nccc(Nc3cnc4cc(F)c(F)cc4c3)n2)c(Cl)cc1O[C@H]1C[C@@H](N(C)C)C1]

### Step 1—Preparation of intermediate (1s,3s)-3-(5-chloro-2-methoxy-4-nitrophenoxy)-N,N-dimethylcyclobutan-1-amine

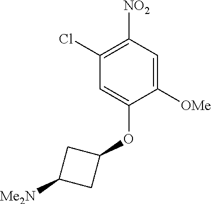 [SMILES: COc1cc([N+](=O)[O-])c(Cl)cc1O[C@H]1C[C@@H](N(C)C)C1]

The title compound was prepared according to general procedure G starting from 1-chloro-5-fluoro-4-methoxy-2-nitrobenzene. MS (m/z): [M+H]⁺−301.4.

### Step 2—Preparation of Intermediate 2-chloro-4-((1s,3s)-3-(dimethylamino)cyclobutoxy)-5-methoxyaniline

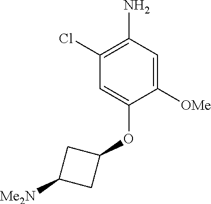 [SMILES: COc1cc(N)c(Cl)cc1O[C@H]1C[C@@H](N(C)C)C1]

The title compound was prepared according to general procedure I. MS (m/z): [M+H]⁺−271.2.

### Step 3—Preparation of N2-(2-chloro-4-((1s,3s)-3-(dimethylamino)cyclobutoxy)-5-methoxyphenyl)-N4-(6,7-difluoroquinolin-3-yl)pyrimidine-2,4-diamine

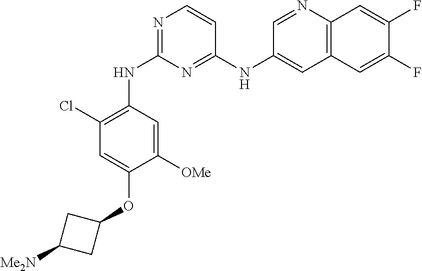 [SMILES: COc1cc(Nc2nccc(Nc3cnc4cc(F)c(F)cc4c3)n2)c(Cl)cc1O[C@H]1C[C@@H](N(C)C)C1]

The title compound was prepared according to the general SNAr procedure E. MS (m/z): [M+H]⁺527.6. ¹H NMR (500 MHz, DMSO) δ 9.91 (s, 1H), 8.82 (s, 1H), 8.75-8.60 (m, 2H), 8.05 (d, J=5.7 Hz, 1H), 7.89 (dd, J=11.5, 8.0 Hz, 1H), 7.30 (s, 1H), 7.20 (s, 1H), 6.99 (s, 1H), 6.27 (d, J=5.7 Hz, 1H), 4.48 (dddd, J=7.3 Hz, 1H), 3.64 (s, 3H), 2.67-2.58 (m, 2H), 2.40-2.30 (m, 1H), 2.05 (s, 6H), 1.90-1.80 (m, 2H).

### Example 304: Preparation of N4-(6,7-difluoroquinolin-3-yl)-N2-(4-((1s,3s)-3-(dimethylamino)cyclobutoxy)-5-methoxy-2-methylphenyl)pyrimidine-2,4-diamine (Compound 154)

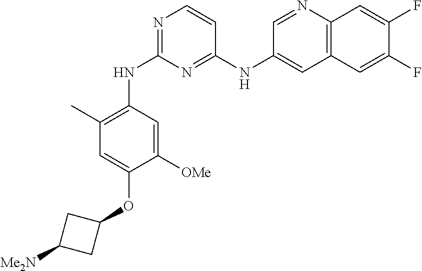 [SMILES: COc1cc(Nc2nccc(Nc3cnc4cc(F)c(F)cc4c3)n2)c(C)cc1O[C@H]1C[C@@H](N(C)C)C1]

### Step 1—Preparation of Intermediate 1-fluoro-2-methoxy-5-methyl-4-nitrobenzene

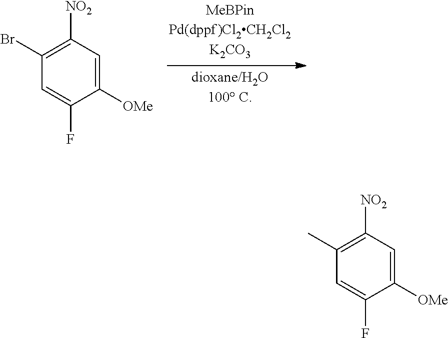

A mixture of 1-bromo-5-fluoro-4-methoxy-2-nitrobenzene (1 equiv.), Pd(dppf)Cl₂·CH₂Cl₂ (10 mol %), K₂CO₃ (3 equiv), and MeBPin (2 equiv.) in dioxane (0.2 M) was heated at 100° C. for 3 hours. The reaction mixture was filtered and concentrated to dryness, then diluted with water and extracted with EtOAc, washed with brine, dried over anhydrous Na₂SO₄, and concentrated to dryness. The crude product was purified by flash chromatography (SiO₂, eluting with EtOAc/Hexanes) to afford the title compound. The reaction mixture was heated at 100° C. for 3 hours.

### Step 2—Preparation of intermediate (1s,3s)-3-(2-methoxy-5-methyl-4-nitrophenoxy)-N,N-dimethylcyclobutan-1-amine

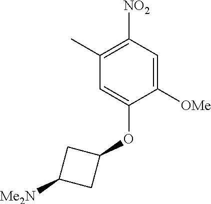 [SMILES: COc1cc([N+](=O)[O-])c(C)cc1O[C@H]1C[C@@H](N(C)C)C1]

The title compound was prepared according to general procedure G. MS (m/z): [M+H]⁺−281.2.

### Step 3—Preparation of Intermediate 4-((1s,3s)-3-(dimethylamino)cyclobutoxy)-5-methoxy-2-methylaniline

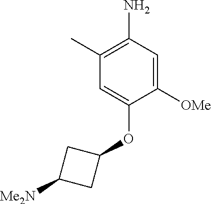 [SMILES: COc1cc(N)c(C)cc1O[C@H]1C[C@@H](N(C)C)C1]

The title compound was prepared according to general procedure H. MS (m/z): [M+H]⁺−251.2.

### Step 4—Preparation of N4-(6,7-difluoroquinolin-3-yl)-N2-(4-((1s,3s)-3-(dimethylamino)cyclobutoxy)-5-methoxy-2-methylphenyl)pyrimidine-2,4-diamine

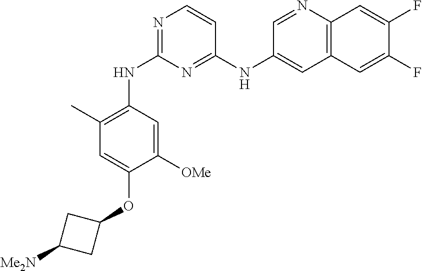 [SMILES: COc1cc(Nc2nccc(Nc3cnc4cc(F)c(F)cc4c3)n2)c(C)cc1O[C@H]1C[C@@H](N(C)C)C1]

The title compound was prepared according to the general SNAr procedure E. MS (m/z): [M+H]⁺507.4. ¹H NMR (500 MHz, DMSO) δ 9.83 (s, 1H), 8.78 (s, 1H), 8.70 (br, 1H), 8.55 (s, 1H), 8.03 (d, J=5.6 Hz, 1H), 7.88 (dd, J=11.5, 7.9 Hz, 1H), 6.96 (s, 1H), 6.80 (s, 1H), 6.22 (d, J=5.7 Hz, 1H), 4.44 (dddd, J=7.5 Hz, 1H), 3.62 (s, 3H), 2.68-2.60 (m, 2H), 2.39-2.31 (m, 1H), 2.13 (s, 3H), 2.07 (s, 6H), 1.93-1.83 (m, 2H).

### Example 305: Preparation of 1-(4-((4-((6,7-difluoroquinolin-3-yl)amino)pyrimidin-2-yl)amino)-2-methoxyphenyl)-4-methylpiperidin-4-ol (Compound 155)

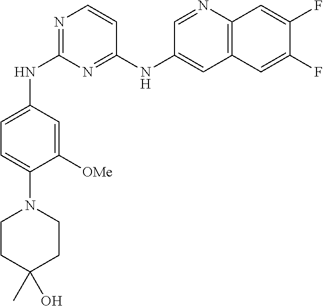 [SMILES: COc1cc(Nc2nccc(Nc3cnc4cc(F)c(F)cc4c3)n2)ccc1N1CCC(C)(O)CC1]

### Step 1—Preparation of Intermediate 1-(2-methoxy-4-nitrophenyl)-4-methylpiperidin-4-ol

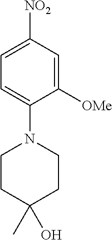 [SMILES: COc1cc([N+](=O)[O-])ccc1N1CCC(C)(O)CC1]

The title compound was prepared according to general procedure G starting from 1-fluoro-2-methoxy-4-nitrobenzene. MS (m/z): [M+H]⁺−267.2.

### Step 2—Preparation of Intermediate 1-(4-amino-2-methoxyphenyl)-4-methylpiperidin-4-ol

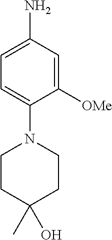 [SMILES: COc1cc(N)ccc1N1CCC(C)(O)CC1]

The title compound was prepared according to general procedure H. MS (m/z): [M+H]⁺−237.0.

### Step 3—Preparation of 1-(4-((4-((6,7-difluoroquinolin-3-yl)amino)pyrimidin-2-yl)amino)-2-methoxyphenyl)-4-methylpiperidin-4-ol

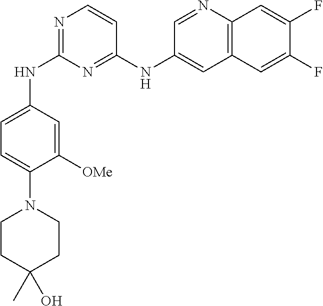 [SMILES: COc1cc(Nc2nccc(Nc3cnc4cc(F)c(F)cc4c3)n2)ccc1N1CCC(C)(O)CC1]

The title compound was prepared according to the general SNAr procedure E. MS (m/z): [M+H]⁺493.4. ¹H NMR (500 MHz, DMSO) δ 9.96 (s, 1H), 9.10-9.01 (m, 2H), 8.95 (s, 1H), 8.11 (d, J=5.7 Hz, 1H), 7.95 (dd, J=11.5, 8.1 Hz, 1H), 7.67 (s, 1H), 7.30-7.15 (m, 2H), 6.89 (d, J=8.5 Hz, 1H), 6.31 (d, J=5.7 Hz, 1H), 4.21 (br, 1H), 3.67 (s, 3H), 3.02-2.90 (m, 4H), 1.71-1.55 (m, 4H), 1.19 (s, 3H).

### Example 306: Preparation of 1-(5-((4-((6,7-difluoroquinolin-3-yl)amino)pyrimidin-2-yl)amino)-3-methoxypyridin-2-yl)-4-methylpiperidin-4-ol (Compound 156)

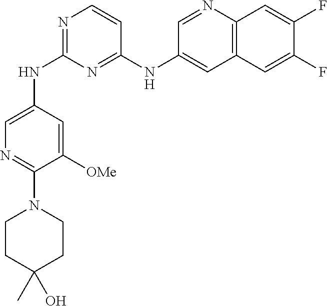 [SMILES: COc1cc(Nc2nccc(Nc3cnc4cc(F)c(F)cc4c3)n2)cnc1N1CCC(C)(O)CC1]

The title compound was prepared according to a similar procedure used for the synthesis of Compound 155 starting from 2-chloro-3-methoxy-5-nitropyridine. MS (m/z): [M+H]⁺−494.4. ¹H NMR (500 MHz, DMSO) δ 9.99 (br, 1H), 9.11 (s, 1H), 9.03 (s, 1H), 8.92 (s, 1H), 8.16 (d, J=2.1 Hz, 1H), 8.10 (d, J=5.7 Hz, 1H), 7.97-7.87 (m, 1H), 7.73 (s, 1H), 7.48 (s, 1H), 6.33 (d, J=5.7 Hz, 1H), 4.22 (s, 1H), 3.67 (s, 3H), 3.45-3.36 (m, 2H), 3.27-3.18 (m, 2H), 1.65-1.53 (m, 4H), 1.17 (s, 3H).

### Example 307: Preparation of N4-(6,7-difluoroquinolin-3-yl)-N2-(6-(2,2-dimethylmorpholino)-5-methoxypyridin-3-yl)pyrimidine-2,4-diamine (Compound 157)

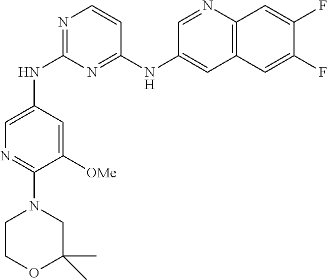 [SMILES: COc1cc(Nc2nccc(Nc3cnc4cc(F)c(F)cc4c3)n2)cnc1N1CCOC(C)(C)C1]

The title compound was prepared according to a similar procedure used for the synthesis of Compound 156. MS (m/z): [M+H]⁺−494.4. ¹H NMR (500 MHz, DMSO) δ 9.99 (s, 1H), 9.16 (s, 1H), 9.04 (s, 1H), 8.90 (s, 1H), 8.20 (d, J=2.1 Hz, 1H), 8.11 (d, J=5.7 Hz, 1H), 7.94 (dd, J=10.8, 8.8 Hz, 1H), 7.74 (s, 1H), 7.51 (s, 1H), 6.34 (d, J=5.7 Hz, 1H), 3.81-3.74 (m, 2H), 3.69 (s, 3H), 3.22-3.17 (m, 2H), 3.11-3.04 (m, 2H), 1.26 (s, 6H).

### Example 308: Preparation of Compound 158

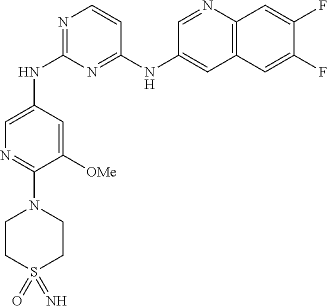 [SMILES: COc1cc(Nc2nccc(Nc3cnc4cc(F)c(F)cc4c3)n2)cnc1N1CCS(=N)(=O)CC1]

### Step 1—Preparation of Intermediate 4-(3-methoxy-5-nitropyridin-2-yl)thiomorpholine

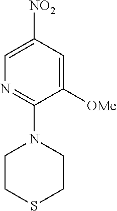 [SMILES: COc1cc([N+](=O)[O-])cnc1N1CCSCC1]

The title compound was prepared according to the general procedure G starting from 2-chloro-3-methoxy-5-nitropyridine.

### Step 2—Preparation of Intermediate 1-imino-4-(3-methoxy-5-nitropyridin-2-yl)-116-thiomorpholine 1-oxide

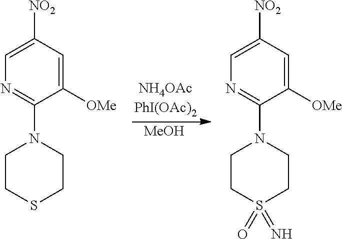 [SMILES: "MeOH" ; COc1cc([N+](=O)[O-])cnc1N1CCSCC1 ; COc1cc([N+](=O)[O-])cnc1N1CCS(=N)(=O)CC1]

To a solution of 4-(3-methoxy-5-nitropyridin-2-yl)thiomorpholine (1 equiv.) in MeOH (0.05 M) was added NH₄OAc (1.5 equiv.) and PhI(OAc)₂ (2.1 equiv.). The reaction mixture was stirred for 16 hours. The mixture was concentrated to dryness and the crude product was purified by flash chromatography (SiO₂, eluting with MeOH/CH₂Cl₂) to afford the title compound. MS (m/z): [M+H]⁺−287.7.

### Step 3—Preparation of Intermediate 4-(5-amino-3-methoxypyridin-2-yl)-1-imino-116-thiomorpholine 1-oxide

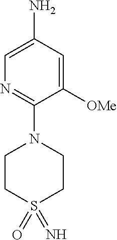 [SMILES: COc1cc(N)cnc1N1CCS(=N)(=O)CC1]

The title compound was prepared according to the general procedure H. MS (m/z): [M+H]⁺−257.3.

### Step 3—Preparation of 4-(5-((4-((6,7-difluoroquinolin-3-yl)amino)pyrimidin-2-yl)amino)-3-methoxypyridin-2-yl)-1-imino-116-thiomorpholine 1-oxide

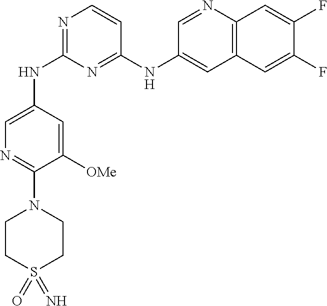 [SMILES: COc1cc(Nc2nccc(Nc3cnc4cc(F)c(F)cc4c3)n2)cnc1N1CCS(=N)(=O)CC1]

The title compound was prepared according to the general SNAr procedure E. MS (m/z): [M+H]⁺513.3. ¹H NMR (500 MHz, DMSO) δ 9.97 (s, 1H), 9.22 (s, 1H), 9.00-8.86 (m, 2H), 8.20 (s, 1H), 8.12 (d, J=5.7 Hz, 1H), 7.95 (dd, J=11.6, 8.0 Hz, 1H), 7.81-7.70 (m, 1H), 7.62 (s, 1H), 6.34 (d, J=5.7 Hz, 1H), 3.91-3.82 (m, 2H), 3.73-3.59 (m, 6H), 3.15-3.02 (m, 4H).

### Example 309: Preparation of 8-fluoro-3-((2-((6-(4-hydroxy-4-methylpiperidin-1-yl)-5-methoxypyridin-3-yl)amino)pyrimidin-4-yl)amino)quinolin-2(1H)-one (Compound 159)

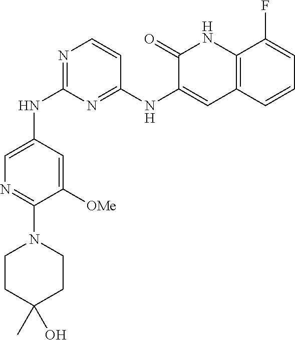 [SMILES: COc1cc(Nc2nccc(Nc3cc4cccc(F)c4[nH]c3=O)n2)cnc1N1CCC(C)(O)CC1]

The title compound was prepared according to a similar procedure used for the synthesis of Compound 155. MS (m/z): [M+H]⁺−492.4. ¹H NMR (500 MHz, DMSO) δ 9.13-9.01 (m, 2H), 8.83 (s, 1H), 8.12 (s, 1H), 8.05 (d, J=5.6 Hz, 1H), 7.45 (s, 1H), 7.24 (s, 1H), 7.13 (s, 1H), 7.02 (s, 1H), 6.70 (s, 1H), 4.25 (s, 1H), 3.70 (s, 3H), 3.46-3.37 (m, J=12.3 Hz, 2H), 3.26-3.14 (m, J=9.9 Hz, 2H), 1.67-1.53 (m, 4H), 1.18 (s, 3H).

### Example 310: Preparation of 3-((2-((6-(2,2-dimethylmorpholino)-5-methoxypyridin-3-yl)amino)pyrimidin-4-yl)amino)-8-fluoroquinolin-2(1H)-one (Compound 160)

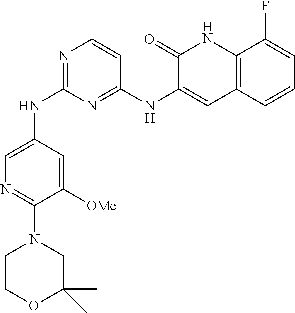 [SMILES: COc1cc(Nc2nccc(Nc3cc4cccc(F)c4[nH]c3=O)n2)cnc1N1CCOC(C)(C)C1]

The title compound was prepared according to a similar procedure used for the synthesis of Compound 155. MS (m/z): [M+H]⁺−492.4. ¹H NMR (500 MHz, DMSO) δ 9.13 (s, 1H), 9.09 (s, 1H), 8.80 (s, 1H), 8.13 (d, J=1.6 Hz, 1H), 8.05 (d, J=5.7 Hz, 1H), 7.54 (s, 1H), 7.20 (s, 1H), 7.15-7.05 (m, 1H), 6.96 (s, 1H), 6.70 (d, J=5.4 Hz, 1H), 3.82-3.75 (m, 2H), 3.71 (s, 3H), 3.22-3.17 (m, 2H), 3.07 (s, 2H), 1.26 (s, 6H).

### Example 311: Preparation of 4-(5-((4-((6,7-difluoroquinolin-3-yl)amino)pyrimidin-2-yl)amino)-3-methoxypyridin-2-yl)thiomorpholine 1,1-dioxide (Compound 161)

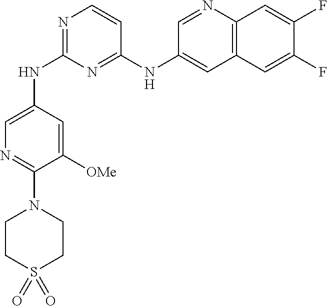 [SMILES: COc1cc(Nc2nccc(Nc3cnc4cc(F)c(F)cc4c3)n2)cnc1N1CCS(=O)(=O)CC1]

The title compound was prepared according to a similar procedure used for the synthesis of Compound 155. MS (m/z): [M+H]⁺−514.3. ¹H NMR (500 MHz, DMSO) δ 10.01 (s, 1H), 9.24 (s, 1H), 8.99 (s, 1H), 8.94 (s, 1H), 8.23 (d, J=1.7 Hz, 1H), 8.12 (d, J=5.7 Hz, 1H), 7.95 (dd, J=11.5, 8.2 Hz, 1H), 7.84-7.71 (m, 1H), 7.65 (s, 1H), 6.35 (d, J=5.7 Hz, 1H), 3.87-3.74 (m, 4H), 3.69 (s, 3H), 3.26-3.18 (m, 4H).

### Example 312: Preparation of 6-(difluoromethyl)quinolin-3-amine hydrochloride (Intermediate 121)

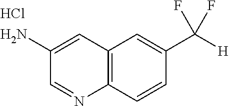 [SMILES: Cl ; Nc1cnc2ccc(C(F)F)cc2c1]

### Step-1

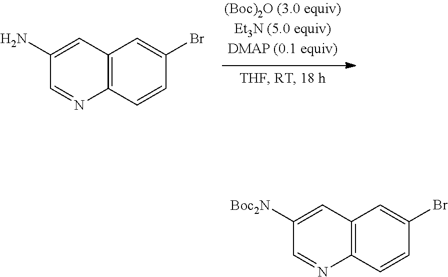

To 6-bromoquinolin-3-amine (400 mg, 1.79 mmol) in THF (17.9 mL) was charged triethylamine (1.26 mL, 8.97 mmol) and 4-dimethylaminopyridine (22.1 mg, 179 umol) and di-tert-butyl dicarbonate (1.24 mL, 5.38 mmol). The reaction mixture was stirred overnight. Ethyl acetate (25 mL), and water (25 mL) was added to the reaction mixture, layers separated, and aqueous layer extracted twice more with ethyl acetate (10 mL each), organics dried over MgSO4, filtered and concentrated under reduced pressure to afford the desired product as a yellow powder. The crude material carried forward to the next step without further purification. MS (m/z): [M+H]⁺=423.1.

### Step-2

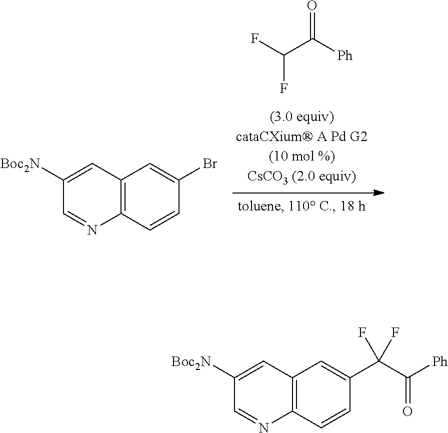

To a solution of di-tert-butyl (6-bromoquinolin-3-yl)iminodicarbonate (1 equiv.), 2,2-difluoroacetophenone (749 uL, 5.38 mmol), cesium carbonate (1.19 g, 3.59 mmol), in toluene (8.97 mL) was added cataCXium® A Pd G2 (120 mg, 179 umol) and the reaction heated to 115° C. for 18 h. The reaction mixture was diluted with ethyl acetate (50 mL), washed with water (25 mL×2), combined aqueous layer extracted once more with ethyl acetate (15 mL), combined organics dried over MgSO4, filtered, and concentrated under reduced pressure to afford the desired product as a brown paste. The compound was carried forward to the next step without further purification. MS (m/z): [M+H]⁺=499.2.

### Step-3

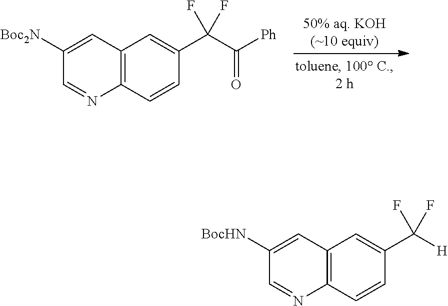

To the above step-2 intermediate (894 mg, 1.79 mmol) in toluene (10.0 mL) was added 50% KOH in Water (2.00 mL, 17.8 mmol). The reaction mixture was placed in a pre-heated oil bath (105° C.), stirring continued for 1 h. The reaction mixture was then concentrated under reduced pressure, and the resulting residue was partitioned between ethyl acetate (25 mL), and water (25 mL), and the aqueous layer was extracted with ethyl acetate (25 mL), combined organics dried over MgSO4, filtered, and concentrated under reduced pressure to afford a pale brown paste. The crude was carried forward to the next step. MS (m/z): [M+H]⁺=295.1.

### Step-4

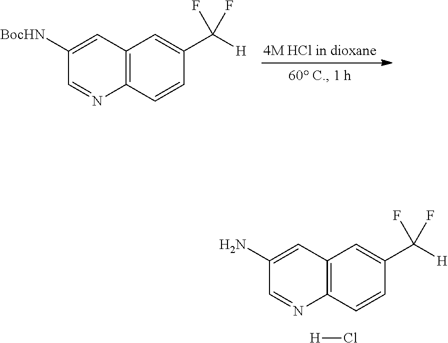

A solution of the step-3 crude material in 4M HCl in dioxane (11.2 mL, 44.7 mmol) and methanol (1 mL) was placed in a pre-heated oil bath (65° C.), stirring continued for 1 h, and the reaction mixture was concentrated under reduced pressure. Purification over reverse phase flash chromatography (wet load in DMF, 60 g, 5-100% ACN in 10 mM AMF mobile phase) afforded 6-(difluoromethyl)quinolin-3-amine hydrochloride (200 mg, 48% over 4-steps) as a pale brown paste. MS (m/z): [M+H]⁺=195.1.

### Example 313: Preparation of Compound 162

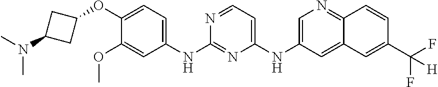 [SMILES: COc1cc(Nc2nccc(Nc3cnc4ccc(C(F)F)cc4c3)n2)ccc1O[C@H]1C[C@H](N(C)C)C1]

Compound 162 was prepared from the corresponding intermediate according to general procedure C. MS (m/z): [M+H]⁺ 507.5. H NMR (400 MHz, DMSO-D6) δ 9.93 (s, 1H), 9.11 (br s, 2H), 9.02 (d, J=2.5 Hz, 1H), 8.10 (d, J=5.7 Hz, 1H), 8.05 (d, J=8.6 Hz, 1H), 8.02 (br s, 1H), 7.72 (dd, J=8.7, 1.7 Hz, 1H), 7.30 (br s, 1H), 7.24 (dd, J=8.5, 1.8 Hz, 1H), 7.21 (t, J=55.8 Hz, 1H), 6.67 (d, J=8.7 Hz, 1H), 6.32 (d, J=5.7 Hz, 1H), 4.69-4.62 (m, 1H), 3.66 (s, 3H), 2.80 (tt, J=7.6, 5.3 Hz, 1H), 2.28 (ddd, J=12.2, 8.3, 4.8 Hz, 2H), 2.17-2.09 (m, 2H), 2.06 (s, 6H).

### Example 314: Preparation of Compound 163

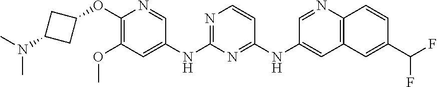 [SMILES: COc1cc(Nc2nccc(Nc3cnc4ccc(C(F)F)cc4c3)n2)cnc1O[C@H]1C[C@@H](N(C)C)C1]

Compound 163 was prepared from the corresponding intermediate according to general procedure C. MS (m/z): [M+H]⁺ 508.3. ¹H NMR (400 MHz, dmso-d6) δ 10.00 (s, 1H), 9.21 (s, 1H), 9.06 (s, 1H), 9.03 (d, J=2.2 Hz, 1H), 8.12 (d, J=5.7 Hz, 1H), 8.08-8.00 (m, 3H), 7.72 (dd, J=8.7, 1.7 Hz, 1H), 7.59 (s, 1H), 7.21 (t, J=55.8 Hz, 1H), 6.35 (d, J=5.7 Hz, 1H), 4.86 (p, J=7.5 Hz, 1H), 3.67 (s, 3H), 2.61-2.52 (m, 2H), 2.39-2.28 (m, 1H), 2.04 (s, 6H), 1.86-1.76 (m, 2H).

### Example 315: Preparation of 6-(4-(dimethylamino)piperidin-1-yl)-5-methoxypyridin-3-amine (Intermediate 122)

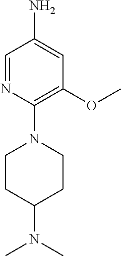 [SMILES: COc1cc(N)cnc1N1CCC(N(C)C)CC1]

### Step 1: 1-(3-methoxy-5-nitropyridin-2-yl)-N,N-dimethylpiperidin-4-amine

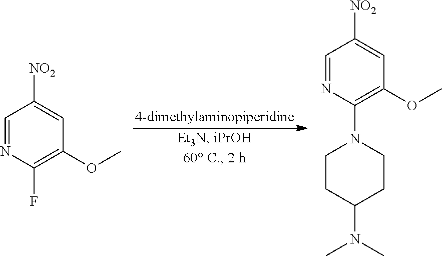 [SMILES: COc1cc([N+](=O)[O-])cnc1F ; "Et3N, iPrOH60° C., 2 h" ; COc1cc([N+](=O)[O-])cnc1N1CCC(N(C)C)CC1]

The title compound was prepared from 2-Chloro-3-methoxy-5-nitropyridine and 4-dimethylaminopiperidine according to the general procedure G. MS (m/z): [M+H]⁺: 281.3

### Step 2: 6-(4-(dimethylamino)piperidin-1-yl)-5-methoxypyridin-3-amine

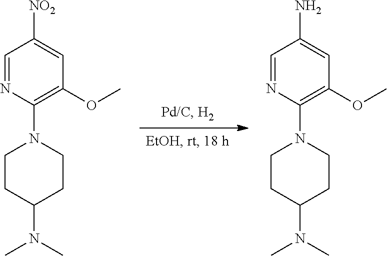 [SMILES: COc1cc([N+](=O)[O-])cnc1N1CCC(N(C)C)CC1 ; "EtOH, rt, 18 h" ; COc1cc(N)cnc1N1CCC(N(C)C)CC1]

The title compound was prepared from 1-(3-methoxy-5-nitropyridin-2-yl)-N,N-dimethylpiperidin-4-amine according to the general procedure H. MS (m/z): [M+H]⁺: 251.3

### Example 316: Preparation of Compound 164

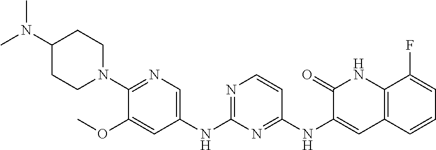 [SMILES: COc1cc(Nc2nccc(Nc3cc4cccc(F)c4[nH]c3=O)n2)cnc1N1CCC(N(C)C)CC1]

Compound 164 was prepared from the corresponding intermediate according to general procedure C. MS (m/z): [M+H]⁺ 504.6. H NMR (500 MHz, DMSO) δ 12.29-12.16 (m, 1H), 9.11 (d, J=16.6 Hz, 2H), 8.92 (s, 1H), 8.12 (d, J=1.9 Hz, 1H), 8.09-8.06 (m, 1H), 7.48 (s, 1H), 7.30-7.20 (m, 2H), 7.10 (s, 1H), 6.76 (d, J=5.7 Hz, 1H), 3.86 (d, J=12.6 Hz, 2H), 3.75-3.67 (m, 3H), 2.72-2.66 (m, 2H), 2.26-2.17 (m, 7H), 1.86-1.82 (m, 2H), 1.56-1.47 (m, 2H).

### Example 317: Preparation of (S)-5-methoxy-6-((1-methylpyrrolidin-3-yl)oxy)pyridin-3-amine (Intermediate 123)

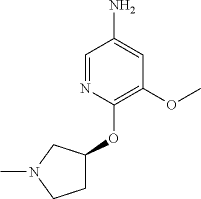

### Step 1: (S)-3-methoxy-2-((1-methylpyrrolidin-3-yl)oxy)-5-nitropyridine

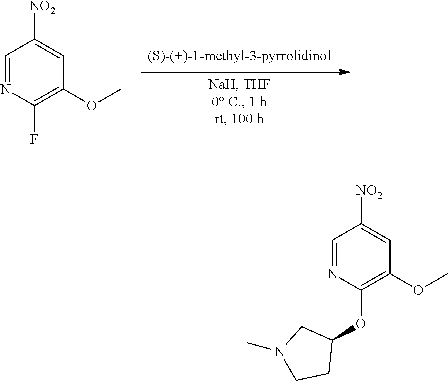

The title compound was prepared following general procedure G from (S)-(+)-1-methyl-3-pyrrolidinol and 2-chloro-3-methoxy-5-nitropyridine. MS (m/z): [M+H]⁺: 254.1

### Step 2: (S)-5-methoxy-6-((1-methylpyrrolidin-3-yl)oxy)pyridin-3-amine

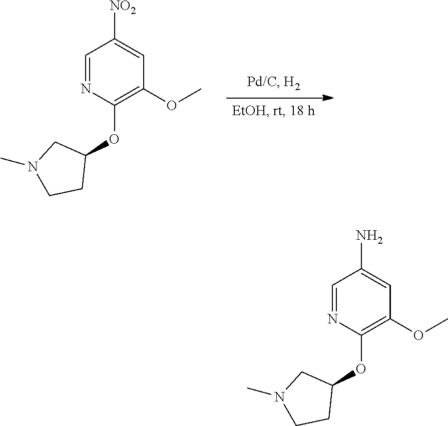

The title compound was prepared from (S)-3-methoxy-2-((1-methylpyrrolidin-3-yl)oxy)-5-nitropyridine according to the general procedure H. MS (m/z): [M+H]⁺: 224.3

### Example 318: Preparation of Compound 165

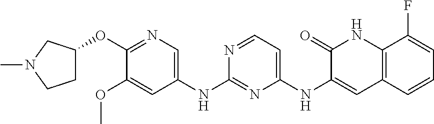 [SMILES: COc1cc(Nc2nccc(Nc3cc4cccc(F)c4[nH]c3=O)n2)cnc1O[C@@H]1CCN(C)C1]

Compound 165 was prepared from the corresponding intermediate according to general procedure C. MS (m/z): [M+H]⁺ 478.3. H NMR (500 MHz, DMSO) δ 12.29 (s, 1H), 9.14 (d, J=20.0 Hz, 2H), 8.93 (s, 1H), 8.10-8.03 (m, 2H), 7.52 (s, 1H), 7.30-7.22 (m, 2H), 7.14 (d, J=4.8 Hz, 1H), 6.80 (d, J=5.7 Hz, 1H), 5.41-5.34 (m, 1H), 3.71 (s, 3H), 2.82-2.78 (m, 1H), 2.70-2.65 (m, 1H), 2.63-2.60 (m, 1H), 2.40-2.35 (m, 1H), 2.32-2.23 (m, 4H), 1.87-1.79 (m, 1H).

### Example 319: Preparation of (R)-5-methoxy-6-((1-methylpyrrolidin-3-yl)oxy)pyridin-3-amine (Intermediate 124)

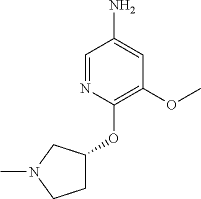

### Step 1: (R)-3-methoxy-2-((1-methylpyrrolidin-3-yl)oxy)-5-nitropyridine

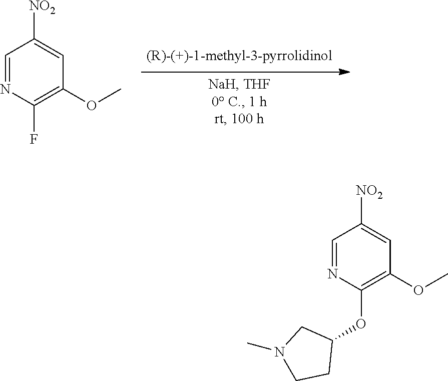

The title compound was prepared following general procedure G from (R)-(−)-1-methyl-3-pyrrolidinol and 2-chloro-3-methoxy-5-nitropyridine. MS (m/z): [M+H]⁺: 254.1.

### Step 2: (R)-5-methoxy-6-((1-methylpyrrolidin-3-yl)oxy)pyridin-3-amine

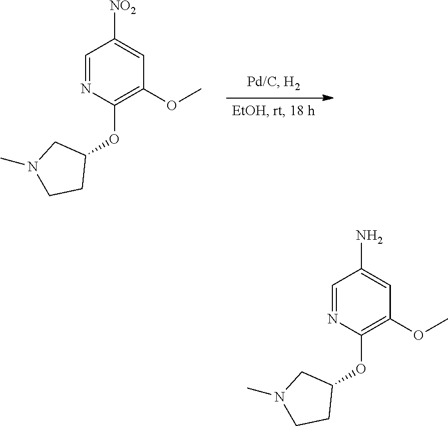

The title compound was prepared from (R)-3-methoxy-2-((1-methylpyrrolidin-3-yl)oxy)-5-nitropyridine according to the general procedure H. MS (m/z): [M+H]⁺: 224.3

### Example 320: Preparation of Compound 166

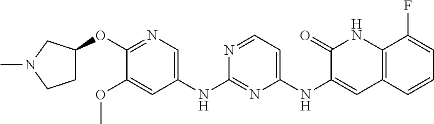 [SMILES: COc1cc(Nc2nccc(Nc3cc4cccc(F)c4[nH]c3=O)n2)cnc1O[C@H]1CCN(C)C1]

Compound 166 was prepared from the corresponding intermediate according to general procedure C. MS (m/z): [M+H]⁺ 478.6. H NMR (500 MHz, DMSO) δ 12.36-12.13 (m, 1H), 9.11 (d, J=7.7 Hz, 1H), 8.91-8.77 (m, 1H), 8.11-8.01 (m, 2H), 7.53 (s, 1H), 7.17 (s, 2H), 7.07-6.98 (m, 1H), 6.73 (s, 1H), 6.66 (s, 1H), 5.42-5.34 (m, 1H), 3.71 (d, J=7.9 Hz, 3H), 2.82-2.78 (m, 1H), 2.70-2.63 (m, 2H), 2.60 (d, J=2.9 Hz, 1H), 2.33-2.19 (m, 4H), 1.87-1.80 (m, 1H).

### Example 321: Preparation of 5-methoxy-6-((2-methyl-2-azaspiro[3.3]heptan-6-yl)oxy)pyridin-3-amine (Intermediate 125)

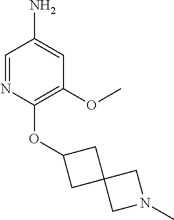 [SMILES: COc1cc(N)cnc1OC1CC2(C1)CN(C)C2]

### Step 1: 2-methyl-2-azaspiro[3.3]heptan-6-ol

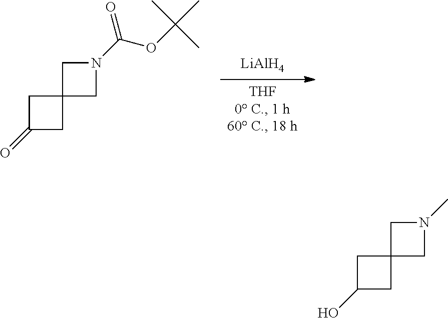

tert-Butyl 6-oxo-2-azaspiro[3.3]heptane-2-carboxylate (1.00 g, 4.73 mmol) was dissolved in THF (9.47 mL) and cooled to 0° C. Lithium aluminium hydride (7.10 mL, 14.2 mmol) was added and the mixture was left to stir for one hour. The temperature was then increased to 60° C. and the mixture was left to stir overnight. The reaction was allowed to cooldown to RT and diluted with Et₂O (20 mL). 0.54 mL of water was added followed by 0.67 mL of aqueous NaOH (3 M). 1.62 mL of water was added again and the mixture was left to stir for 15 min at room temperature. Magnesium sulfate was added and the mixture was left to stir for 15 more minutes. The solid was filtered and the filtrate was concentrated in order vacuum to afford the title compound. ¹H NMR (400 MHz, CDCl₃) δ 4.20-4.07 (m, 1H), 3.16 (d, J=6.0 Hz, 4H), 2.54-2.41 (m, 2H), 2.25 (s, 3H), 2.24 (s, 1H), 2.02-1.95 (m, 2H).

### Step 2: 6-((3-methoxy-5-nitropyridin-2-yl)oxy)-2-methyl-2-azaspiro[3.3]heptane

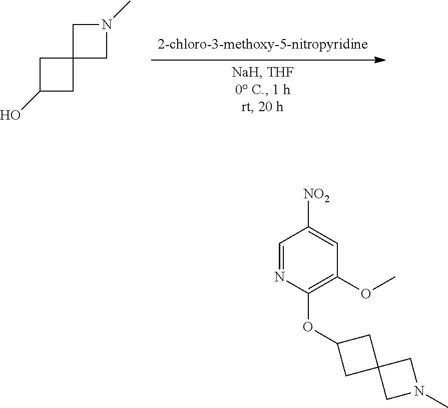

The title compound was prepared from 2-methyl-2-azaspiro[3.3]heptan-6-ol and 2-chloro-3-methoxy-5-nitropyridine according to the general procedure G. MS (m/z): [M+H]⁺: 281.3

### Step 3: 5-methoxy-6-((2-methyl-2-azaspiro[3.3]heptan-6-yl)oxy)pyridin-3-amine

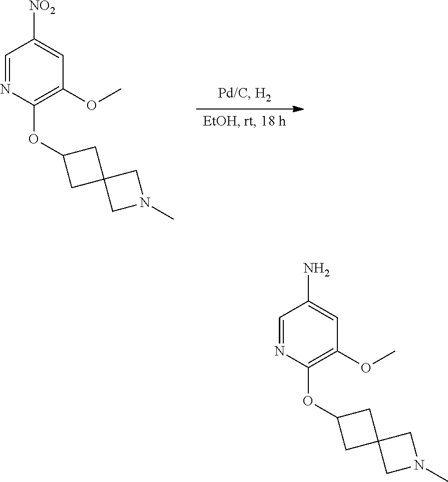 [SMILES: COc1cc([N+](=O)[O-])cnc1OC1CC2(C1)CN(C)C2 ; "EtOH, rt, 18 h" ; "Pd/C, H2" ; COc1cc(N)cnc1OC1CC2(C1)CN(C)C2]

The title compound was prepared from (R)-3-methoxy-2-((1-methylpyrrolidin-3-yl)oxy)-5-nitropyridine according to the general procedure H. MS (m/z): [M+H]⁺: 251.3

### Example 322: Preparation of Compound 167

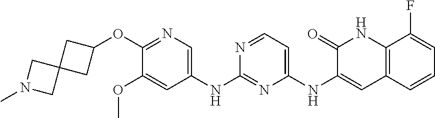 [SMILES: COc1cc(Nc2nccc(Nc3cc4cccc(F)c4[nH]c3=O)n2)cnc1OC1CC2(C1)CN(C)C2]

Compound 167 was prepared from the corresponding intermediate according to general procedure C. MS (m/z): [M+H]⁺ 504.3. H NMR (500 MHz, DMSO) δ 12.29 (s, 1H), 9.13 (d, J=19.9 Hz, 2H), 8.97-8.88 (m, 1H), 8.08 (d, J=5.7 Hz, 1H), 8.01 (s, 1H), 7.53 (s, 1H), 7.29-7.21 (m, 2H), 7.14 (s, 1H), 6.79 (d, J=5.8 Hz, 1H), 5.07-5.02 (m, 1H), 3.72 (d, J=18.8 Hz, 3H), 3.29 (s, 1H), 3.24 (d, J=13.5 Hz, 2H), 3.16 (s, 2H), 2.61-2.56 (m, 2H), 2.23-2.14 (m, 4H).

### Example 323: Preparation of N4-(7-chloro-1,8-naphthyridin-2-yl)-N2-(4-((1s,3s)-3-(dimethylamino)cyclobutoxy)-3-methoxyphenyl)pyrimidine-2,4-diamine (Compound 168)

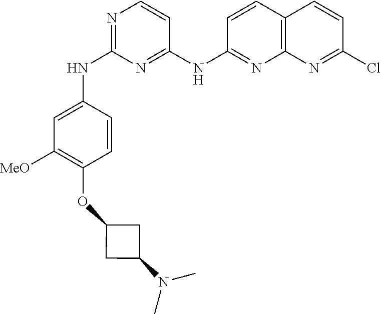 [SMILES: COc1cc(Nc2nccc(Nc3ccc4ccc(Cl)nc4n3)n2)ccc1O[C@H]1C[C@@H](N(C)C)C1]

To a solution of 4-chloro-N-(4-((1s,3s)-3-(dimethylamino)cyclobutoxy)-3-methoxyphenyl)pyrimidin-2-amine (30.0 mg, 86.0 umol) dissolved in DMF (433 uL) was added 7-chloro-1,8-naphthyridin-2-amine (10.8 mg, 57.3 umol) and stirred. Cesium carbonate (76.2 mg, 229 umol) and cesium fluoride (17.4 mg, 115 uL) were added and the reaction was stirred for 16 h at 80° C. After completion of the reaction, the crude product was purified by Prep-MS eluting with a solvent gradient of 35-55% MeCN/H2O (0.1% of 10 mM ammonium bicarbonate buffer). The clean fractions were combined and lyophilized to afford the title product (2.50 mg, 8.7%) as a white solid. MS (m/z): [M+H]⁺−492.6, 494.3. ¹H NMR (500 MHz, MeOD) δ (ppm): 8.27 (s, 1H), 8.21-8.19 (m, 1H), 7.97 (d, J=8.9 Hz, 1H), 7.52 (d, J=5.7 Hz, 1H), 7.47-7.42 (m, 1H), 7.25 (d, J=2.4 Hz, 1H), 7.19-7.16 (m, 1H), 6.85-6.81 (m, 1H), 4.45-4.40 (m, 1H), 3.82 (s, 3H), 3.75-3.64 (m, 2H), 2.70-2.67 (m, 1H), 2.50-2.41 (m, 2H), 2.35-2.30 (m, 1H), 2.23-2.10 (m, 6H), 2.06-2.00 (m, 2H).

### Example 324: Preparation of N-(2-chloropyrimidin-4-yl)-6,7-difluoroquinolin-3-amine (Intermediate 126)

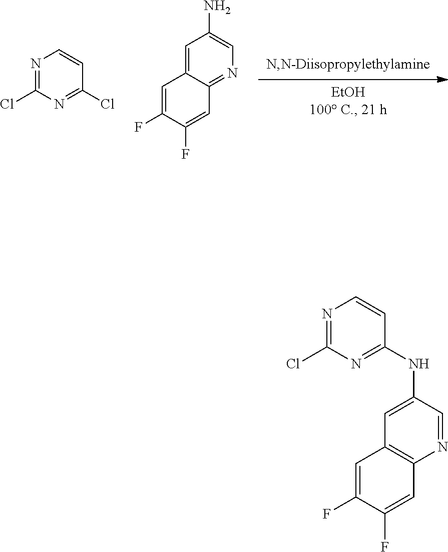

The title compound was prepared according to the SNAr general procedure B starting from 6,7-difluoroquinolin-3-amine and 2,4-dichloropyrimidine. MS (m/z): [M+H]⁺−293.1.

### Example 325: Preparation of Compound 169

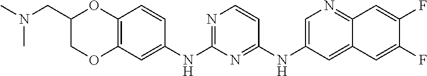 [SMILES: CN(C)CC1COc2cc(Nc3nccc(Nc4cnc5cc(F)c(F)cc5c4)n3)ccc2O1]

Compound 169 was prepared from the corresponding intermediate according to general procedure C. MS (m/z): [M+H]⁺ 465.5. H NMR (500 MHz, DMSO) δ 9.90 (s, 1H), 9.07 (s, 1H), 9.00 (s, 1H), 8.94-8.87 (m, 1H), 8.11-8.05 (m, 1H), 7.96-7.91 (m, 1H), 7.77 (s, 1H), 7.38-7.33 (m, 1H), 7.06-6.99 (m, 1H), 6.79 (d, J=8.7 Hz, 1H), 6.30 (d, J=5.7 Hz, 1H), 4.32-4.25 (m, 2H), 3.96-3.91 (m, 1H), 2.54-2.51 (m, 2H), 2.22 (s, 6H).

### Example 326: Preparation of 6-chloro-N-(2-chloropyrimidin-4-yl)quinolin-3-amine (Intermediate 127)

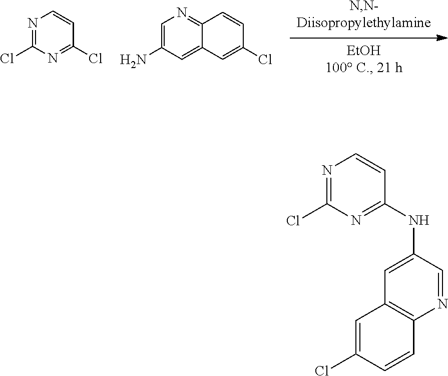 [SMILES: Clc1ccnc(Cl)n1 ; Nc1cnc2ccc(Cl)cc2c1 ; "N,N-Diisopropylethylamine" ; "EtOH100° C., 21 h" ; Clc1ccc2ncc(Nc3ccnc(Cl)n3)cc2c1]

The title compound was prepared according to the SNAr general procedure B starting from 6-chloroquinolin-3-amine and 2,4-dichloropyrimidine. MS (m/z): [M+H]⁺−291.0, 295.0.

### Example 327: Preparation of Compound 170

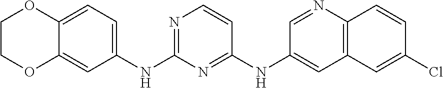 [SMILES: Clc1ccc2ncc(Nc3ccnc(Nc4ccc5c(c4)OCCO5)n3)cc2c1]

Compound 170 was prepared from the corresponding intermediate according to general procedure C. MS (m/z): [M+H]⁺ 406.6, 408.4. H NMR (500 MHz, DMSO) δ 9.93 (s, 1H), 9.09 (s, 1H), 9.03 (s, 1H), 8.89 (d, J=2.3 Hz, 1H), 8.10 (d, J=5.7 Hz, 1H), 7.93 (d, J=8.9 Hz, 1H), 7.89 (s, 1H), 7.59-7.56 (m, 1H), 7.38 (d, J=1.9 Hz, 1H), 7.02 (d, J=7.6 Hz, 1H), 6.79 (d, J=8.7 Hz, 1H), 6.31 (d, J=5.7 Hz, 1H), 4.24 (s, 4H).

### Example 328: Preparation of N4-(6-chloroquinolin-3-yl)-N2-(4-((1s,3s)-3-(dimethylamino)cyclobutoxy)-3-methoxyphenyl)-5-fluoropyrimidine-2,4-diamine (Compound 210)

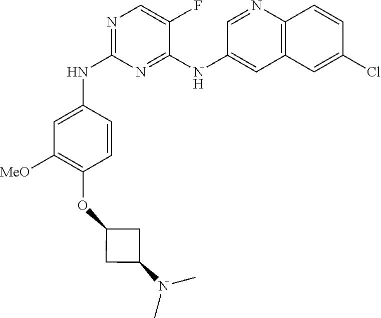 [SMILES: COc1cc(Nc2ncc(F)c(Nc3cnc4ccc(Cl)cc4c3)n2)ccc1O[C@H]1C[C@@H](N(C)C)C1]

### Step 1: 6-chloro-N-(2-chloro-5-fluoropyrimidin-4-yl)quinolin-3-amine

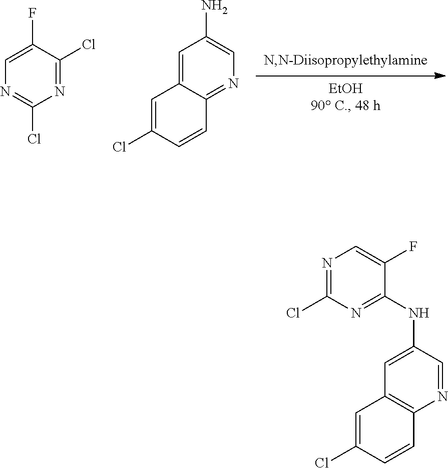 [SMILES: Fc1cnc(Cl)nc1Cl ; Nc1cnc2ccc(Cl)cc2c1 ; "EtOH90° C., 48 h" ; "N,N-Diisopropylethylamine" ; Fc1cnc(Cl)nc1Nc1cnc2ccc(Cl)cc2c1]

The title compound was prepared according to the coupling general procedure B starting from 2,4-Dichloro-5-fluoropyrimidine and 6-chloroquinolin-3-amine. MS (m/z): [M+H]⁺−309.5, 311.1.

### Step 2—Preparation of N4-(6-chloroquinolin-3-yl)-N2-(4-((1s,3s)-3-(dimethylamino)cyclobutoxy)-3-methoxyphenyl)-5-fluoropyrimidine-2,4-diamine

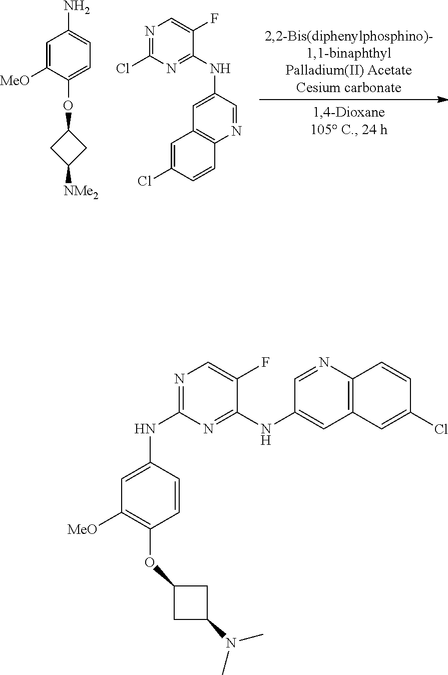

The title compound was prepared according to the coupling general procedure F starting from 4-((1s,3s)-3-(dimethylamino)cyclobutoxy)-3-methoxyaniline and 6-chloro-N-(2-chloro-5-fluoropyrimidin-4-yl)quinolin-3-amine. MS (m/z): [M+H]⁺−509.5, 511.6. ¹H NMR (400 MHz, dmso) δ (ppm): 9.89 (s, 1H), 9.15 (d, J=2.5 Hz, 1H), 9.10 (s, 1H), 8.98 (s, 1H), 8.18 (d, J=3.6 Hz, 1H), 7.96 (d, J=8.9 Hz, 1H), 7.87 (s, 1H), 7.64-7.60 (m, 1H), 7.20 (d, J=8.3 Hz, 2H), 6.71 (d, J=8.4 Hz, 1H), 4.34-4.29 (m, 1H), 3.62 (s, 3H), 2.68-2.64 (m, 1H), 2.33-2.27 (m, 2H), 2.04 (s, 6H), 1.84-1.79 (m, 2H).

### Example 329: Preparation of 3-((2-((2-((dimethylamino)methyl)-2,3-dihydrobenzo[b][1,4]dioxin-6-yl)amino)pyrimidin-4-yl)amino)quinoline-2-carboxamide (Compound 171)

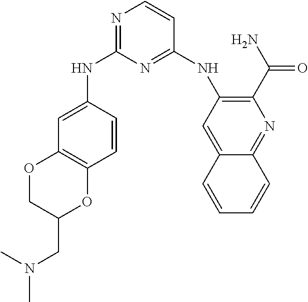 [SMILES: CN(C)CC1COc2cc(Nc3nccc(Nc4cc5ccccc5nc4C(N)=O)n3)ccc2O1]

### Step 1—Preparation of ethyl 3-((2-((2-((dimethylamino)methyl)-2,3-dihydrobenzo[b][1,4]dioxin-6-yl)amino)pyrimidin-4-yl)amino)quinoline-2-carboxylate

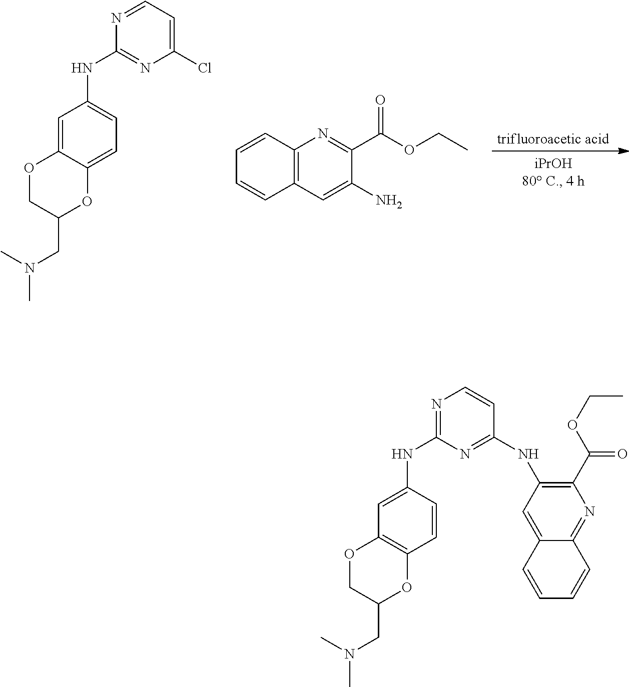 [SMILES: CN(C)CC1COc2cc(Nc3nccc(Cl)n3)ccc2O1 ; CCOC(=O)c1nc2ccccc2cc1N ; "iPrOH80° C., 4 h" ; "trifluoroacetic acid" ; CCOC(=O)c1nc2ccccc2cc1Nc1ccnc(Nc2ccc3c(c2)OCC(CN(C)C)O3)n1]

The title compound was prepared according to the coupling general procedure C starting from 4-chloro-N-(2-((dimethylamino)methyl)-2,3-dihydrobenzo[b][1,4]dioxin-6-yl)pyrimidin-2-amine and ethyl 3-aminoquinoline-2-carboxylate. MS (m/z): [M+H]⁺−501.4.

### Step 2—Preparation of 3-((2-((2-((dimethylamino)methyl)-2,3-dihydrobenzo[b][1,4]dioxin-6-yl)amino)pyrimidin-4-yl)amino)quinoline-2-carboxamide

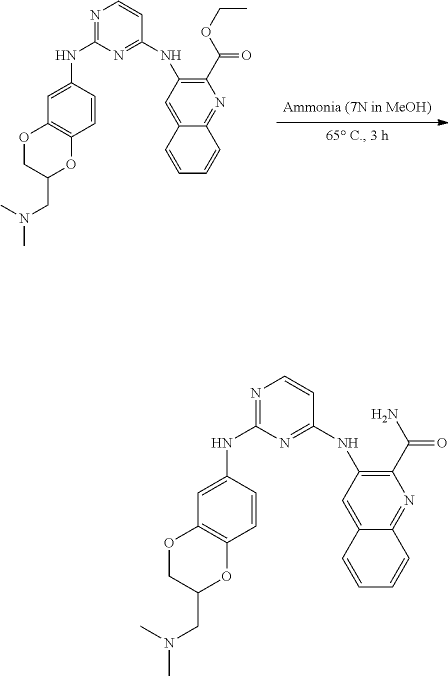 [SMILES: CCOC(=O)c1nc2ccccc2cc1Nc1ccnc(Nc2ccc3c(c2)OCC(CN(C)C)O3)n1 ; "65° C., 3 h" ; "Ammonia (7N in MeOH)" ; CN(C)CC1COc2cc(Nc3nccc(Nc4cc5ccccc5nc4C(N)=O)n3)ccc2O1]

The above intermediate (60.0 mg, 120 umol) was dissolved in ammonia (7 N in MeOH) (1 mL, 106 mmol) and stirred. The reaction was heated in a sealed vial to 65° C. for 3 h. The reaction was cooled in a dry-ice acetone bath and then evaporated to dryness at room temperature. The reaction was re-dissolved in 30% NH40H and stirred overnight in a sealed vial at room temperature. After completion of the reaction solvent was evaporated and the crude product was purified by HPLC Prep-MS eluting with a solvent gradient of 40-60% MeCN/H2O (0.1% of 10 mM ammonium bicarbonate buffer). The clean fractions were combined and lyophilized to afford the title product (31.0 mg, 54%) as a white solid. MS (m/z): [M+H]⁺ 472.5. H NMR (500 MHz, DMSO) δ (ppm): 11.80 (s, 1H), 9.62 (s, 1H), 9.18 (s, 1H), 8.76 (s, 1H), 8.16 (s, 1H), 8.1 (d, J=5.6 Hz, 1H), 8.02-7.99 (m, 1H), 7.86 (s, 1H), 7.70-7.64 (m, 2H), 7.36 (s, 1H), 7.08 (d, J=6.6 Hz, 1H), 6.81 (d, J=8.7 Hz, 1H), 6.31 (d, J=5.7 Hz, 1H), 4.31-4.25 (m, 2H), 3.97-3.92 (m, 1H), 2.53-2.51 (m, 2H), 2.24 (s, 6H).

### Example 330: Preparation of 3-((2-((6-((1s,3s)-3-(dimethylamino)cyclobutoxy)-5-methoxypyridin-3-yl)amino)pyrimidin-4-yl)amino)quinoline-2-carboxamide (Compound 172)

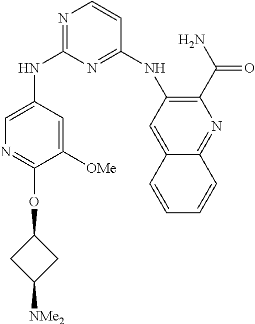 [SMILES: COc1cc(Nc2nccc(Nc3cc4ccccc4nc3C(N)=O)n2)cnc1O[C@H]1C[C@@H](N(C)C)C1]

### Step 1—Preparation of ethyl 3-((2-((6-((1s,3s)-3-(dimethylamino)cyclobutoxy)-5-methoxypyridin-3-yl)amino)pyrimidin-4-yl)amino)quinoline-2-carboxylate

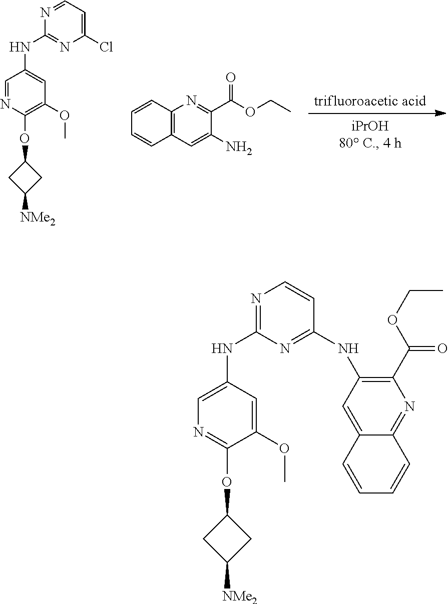 [SMILES: COc1cc(Nc2nccc(Cl)n2)cnc1O[C@H]1C[C@@H](N(C)C)C1 ; CCOC(=O)c1nc2ccccc2cc1N ; "iPrOH80° C., 4 h" ; "trifluoroacetic acid" ; CCOC(=O)c1nc2ccccc2cc1Nc1ccnc(Nc2cnc(O[C@H]3C[C@@H](N(C)C)C3)c(OC)c2)n1]

The title compound was prepared according to the coupling general procedure C starting from 4-chloro-N-(6-((1s,3s)-3-(dimethylamino)cyclobutoxy)-5-methoxypyridin-3-yl)pyrimidin-2-amine and ethyl 3-aminoquinoline-2-carboxylate. MS (m/z): [M+H]⁺−530.5.

### Step 2—Preparation of 3-((2-((6-((1s,3s)-3-(dimethylamino)cyclobutoxy)-5-methoxypyridin-3-yl)amino)pyrimidin-4-yl)amino)quinoline-2-carboxamide

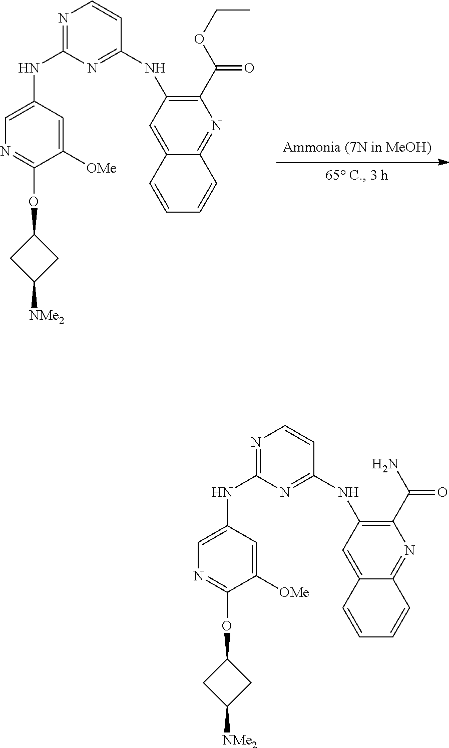 [SMILES: CCOC(=O)c1nc2ccccc2cc1Nc1ccnc(Nc2cnc(O[C@H]3C[C@@H](N(C)C)C3)c(OC)c2)n1 ; "65° C., 3 h" ; "Ammonia (7N in MeOH)" ; COc1cc(Nc2nccc(Nc3cc4ccccc4nc3C(N)=O)n2)cnc1O[C@H]1C[C@@H](N(C)C)C1]

The above intermediate (70.0 mg, 132 umol) was dissolved in ammonia (7 N in MeOH) (1 mL, 117 mmol) and stirred. The reaction was heated in a sealed vial to 65° C. for 3 h. The reaction was cooled in a dry-ice acetone bath and then evaporated to dryness at room temperature. The reaction was re-dissolved in 30% NH₄OH and stirred overnight in a sealed vial at room temperature. The reaction was again evaporated to dryness and the crude product was purified by HPLC Prep-MS eluting with a solvent gradient of 35-55% MeCN/H2O (0.1% of 10 mM ammonium bicarbonate buffer). The clean fractions were combined and lyophilized to afford the title product (10.0 mg, 15%) as a white solid. MS (m/z): [M+H]⁺−501.6. ¹H NMR (500 MHz, DMSO) δ (ppm): 11.85 (s, 1H), 9.60-9.54 (m, 1H), 9.23 (s, 1H), 8.76 (s, 1H), 8.17 (s, 1H), 8.13 (d, J=5.7 Hz, 1H), 8.06 (d, J=2.2 Hz, 1H), 8.01 (d, J=8.5 Hz, 1H), 7.83-7.78 (m, 1H), 7.70-7.67 (m, 1H), 7.65-7.62 (m, 1H), 7.54 (s, 1H), 6.34 (d, J=5.7 Hz, 1H), 4.91-4.88 (m, 1H), 3.69 (s, 3H), 2.64-2.60 (m, 2H), 2.40-2.37 (m, 1H), 2.07 (s, 6H), 1.89-1.83 (m, 2H).

### Example 331: Preparation of N4-(6,7-difluoroquinolin-3-yl)-N2-(4-((1s,3s)-3-(dimethylamino)cyclobutoxy)-3-methoxyphenyl)-5-fluoropyrimidine-2,4-diamine (Compound 173)

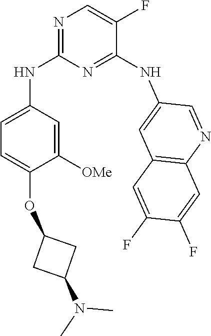 [SMILES: COc1cc(Nc2ncc(F)c(Nc3cnc4cc(F)c(F)cc4c3)n2)ccc1O[C@H]1C[C@@H](N(C)C)C1]

### Step 1—Preparation of N-(2-chloro-5-fluoropyrimidin-4-yl)-6,7-difluoroquinolin-3-amine

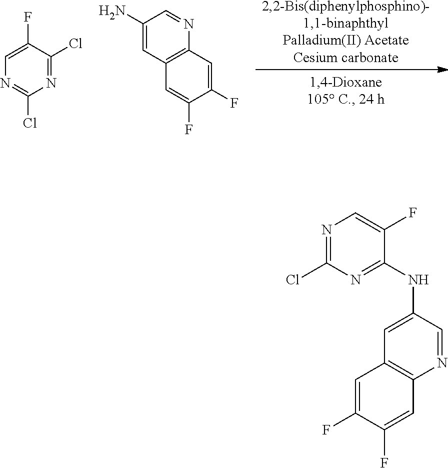 [SMILES: Fc1cnc(Cl)nc1Cl ; Nc1cnc2cc(F)c(F)cc2c1 ; "1,4-Dioxane105° C., 24 h" ; "2,2-Bis(diphenylphosphino)-1,1-binaphthylPalladium(II) AcetateCesium carbonate" ; Fc1cc2cc(Nc3nc(Cl)ncc3F)cnc2cc1F]

The title compound was prepared according to the coupling general procedure F starting from 6,7-difluoroquinolin-3-amine and 2,4-dichloro-5-fluoropyrimidine. MS (m/z): [M−H]⁻−309.2, 311.1.

### Step 2—Preparation of N4-(6,7-difluoroquinolin-3-yl)-N2-(4-((1s,3s)-3-(dimethylamino)cyclobutoxy)-3-methoxyphenyl)-5-fluoropyrimidine-2,4-diamine

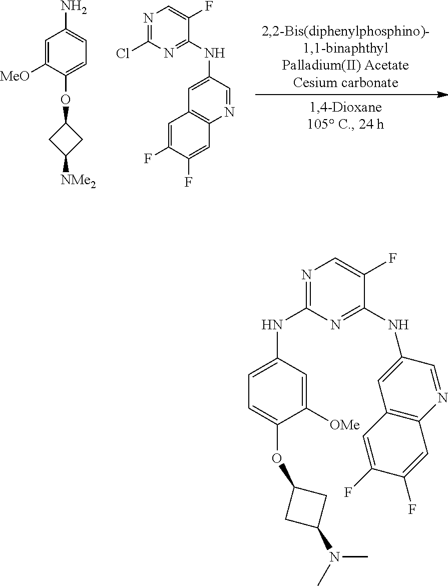

The title compound was prepared according to the coupling general procedure F starting from 4-((1s,3s)-3-(dimethylamino)cyclobutoxy)-3-methoxyaniline and N-(2-chloro-5-fluoropyrimidin-4-yl)-6,7-difluoroquinolin-3-amine. MS (m/z): [M+H]⁺−511.4. ¹H NMR (500 MHz, DMSO) δ (ppm): 9.88 (s, 1H), 9.13 (s, 1H), 9.02 (s, 1H), 8.91 (s, 1H), 8.14 (s, 1H), 7.97-7.91 (m, 1H), 7.72 (s, 1H), 7.20-7.14 (m, 2H), 6.70 (d, J=8.6 Hz, 1H), 4.33-4.27 (m, 1H), 3.62 (s, 3H), 2.57-2.52 (m, 2H), 2.33-2.27 (m, 1H), 2.08-1.99 (m, 6H), 1.84-1.78 (m, 2H).

### Example 332: Preparation of 8-fluoro-3-((2-((5-methoxy-6-(4-methyl-3-(trifluoromethyl)piperazin-1-yl)pyridin-3-yl)amino)pyrimidin-4-yl)amino)qiuinolin-2(1H)-one (Compound 174)

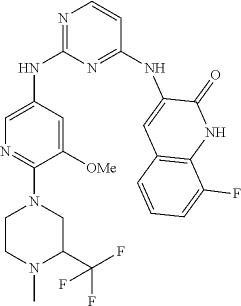 [SMILES: COc1cc(Nc2nccc(Nc3cc4cccc(F)c4[nH]c3=O)n2)cnc1N1CCN(C)C(C(F)(F)F)C1]

### Step 1—Preparation of 4-(3-methoxy-5-nitropyridin-2-yl)-1-methyl-2-(trifluoromethyl)piperazine

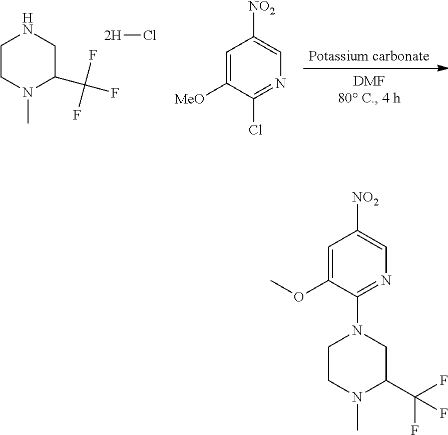

The title compound was prepared according to the coupling general procedure G starting from 2-chloro-3-methoxy-5-nitropyridine and 1-methyl-2-(trifluoromethyl)piperazine hydrochloride. MS (m/z): [M+H]⁺−321.1.

### Step 2—Preparation of 5-methoxy-6-(4-methyl-3-(trifluoromethyl)piperazin-1-yl)pyridin-3-amine

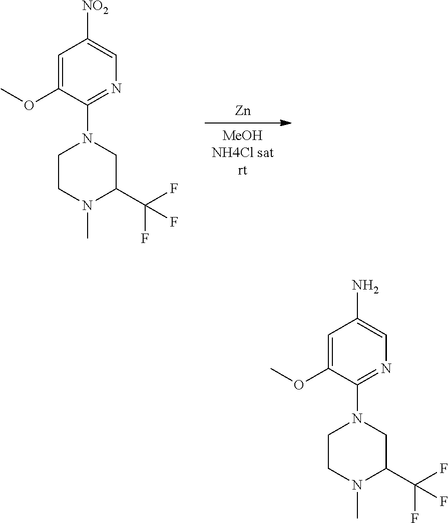

The title compound was prepared according to the coupling general procedure I starting from 4-(3-methoxy-5-nitropyridin-2-yl)-1-methyl-2-(trifluoromethyl)piperazine. MS (m/z): [M+H]⁺−291.2.

### Step 3—Preparation of 8-fluoro-3-((2-((5-methoxy-6-(4-methyl-3-(trifluoromethyl)piperazin-1-yl)pyridin-3-yl)amino)pyrimidin-4-yl)amino)quinolin-2(1H)-one

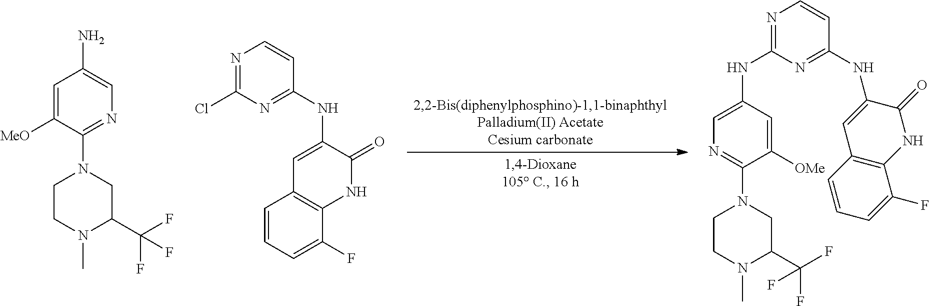 [SMILES: COc1cc(N)cnc1N1CCN(C)C(C(F)(F)F)C1 ; O=c1[nH]c2c(F)cccc2cc1Nc1ccnc(Cl)n1 ; "1,4-Dioxane105° C., 16 h" ; "2,2-Bis(diphenylphosphino)-1,1-binaphthylPalladium(II) AcetateCesium carbonate" ; COc1cc(Nc2nccc(Nc3cc4cccc(F)c4[nH]c3=O)n2)cnc1N1CCN(C)C(C(F)(F)F)C1]

The title compound was prepared according to the coupling general procedure F starting from 5-methoxy-6-(4-methyl-3-(trifluoromethyl)piperazin-1-yl)pyridin-3-amine and 3-((2-chloropyrimidin-4-yl)amino)-8-fluoroquinolin-2(1H)-one. MS (m/z): [M+H]⁺−545.5. ¹HNMR (500 MHz, MeOD) δ (ppm): 8.96 (s, 1H), 8.55-8.53 (m, 1H), 8.11-8.06 (m, 2H), 7.63 (s, 1H), 7.25-7.15 (m, 2H), 6.51 (d, J=5.9 Hz, 1H), 4.59 (s, 3H), 3.80 (s, 2H), 3.48-3.45 (m, 1H), 3.20-3.15 (m, 2H), 2.55 (s, 2H), 1.31 (s, 3H).

### Example 333: Preparation of 8-fluoro-3-((2-((3-methoxy-4-(3,3,4-trimethylpiperazin-1-yl)phenyl)amino)pyrimidin-4-yl)amino)quinolin-2(1H)-one (Compound 175)

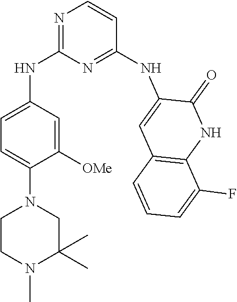 [SMILES: COc1cc(Nc2nccc(Nc3cc4cccc(F)c4[nH]c3=O)n2)ccc1N1CCN(C)C(C)(C)C1]

### Step 1—Preparation of 4-(2-methoxy-4-nitrophenyl)-1,2,2-trimethylpiperazine

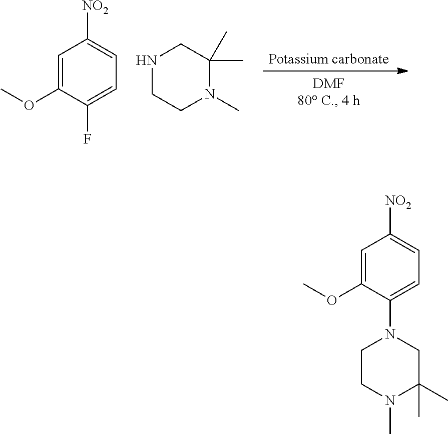

The title compound was prepared according to the coupling general procedure G starting from 1,2,2-Trimethylpiperazine and 2-chloro-3-methoxy-5-nitropyridine. MS (m/z): [M+H]⁺−280.5.

### Step 2—Preparation of 3-methoxy-4-(3,3,4-trimethylpiperazin-1-yl)aniline

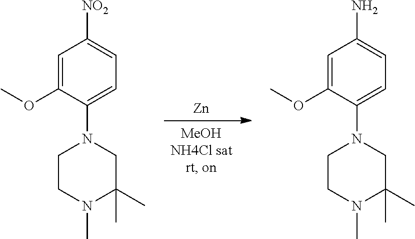

The title compound was prepared according to the coupling general procedure I starting from 4-(2-methoxy-4-nitrophenyl)-1,2,2-trimethylpiperazine. MS (m/z): [M+H]⁺−250.3.

### Step 3—Preparation of 8-fluoro-3-((2-((3-methoxy-4-(3,3,4-trimethylpiperazin-1-yl)phenyl)amino)pyrimidin-4-yl)amino)quinolin-2(1H)-one

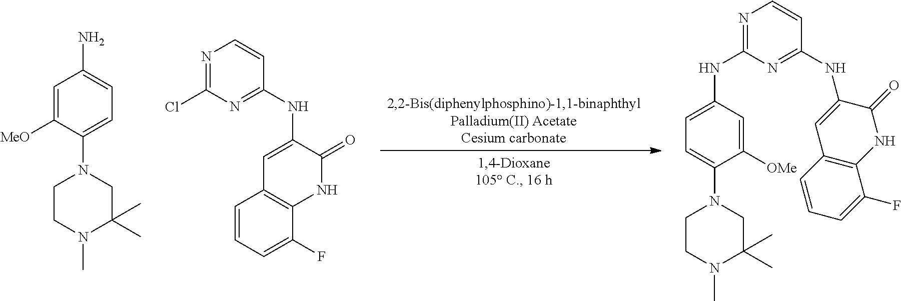 [SMILES: COc1cc(N)ccc1N1CCN(C)C(C)(C)C1 ; O=c1[nH]c2c(F)cccc2cc1Nc1ccnc(Cl)n1 ; "1,4-Dioxane105° C., 16 h" ; "2,2-Bis(diphenylphosphino)-1,1-binaphthylPalladium(II) AcetateCesium carbonate" ; COc1cc(Nc2nccc(Nc3cc4cccc(F)c4[nH]c3=O)n2)ccc1N1CCN(C)C(C)(C)C1]

The title compound was prepared according to the coupling general procedure F starting from 3-methoxy-4-(3,3,4-trimethylpiperazin-1-yl)aniline and 3-((2-chloropyrimidin-4-yl)amino)-8-fluoroquinolin-2(1H)-one. MS (m/z): [M+H]⁺−504.4. ¹H NMR (500 MHz, DMSO) δ (ppm): 12.28 (s, 1H), 9.15 (s, 1H), 9.10 (d, J=4.0 Hz, 1H), 9.06 (s, 1H), 8.50-8.47 (m, 1H), 8.08-8.07 (m, 1H), 8.01 (s, 1H), 7.28-7.20 (m, 3H), 6.81-6.74 (m, 2H), 3.69 (d, J=5.2 Hz, 3H), 2.94 (s, 2H), 2.70-2.62 (m, 2H), 2.59-2.57 (m, 2H), 2.17 (s, 3H), 1.06 (s, 6H).

### Example 334: Preparation of N4-(6,7-difluoroquinolin-3-yl)-N2-(5-methoxy-6-(3-(trifluoromethyl)piperazin-1-yl)pyridin-3-yl)pyrimidine-2,4-diamine (Compound 176)

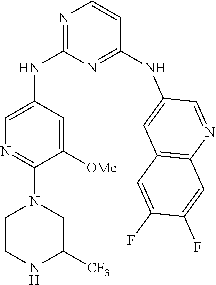 [SMILES: COc1cc(Nc2nccc(Nc3cnc4cc(F)c(F)cc4c3)n2)cnc1N1CCNC(C(F)(F)F)C1]

### Step 1—Preparation of tert-butyl 4-(3-methoxy-5-nitropyridin-2-yl)-2-(trifluoromethyl)piperazine-1-carboxylate

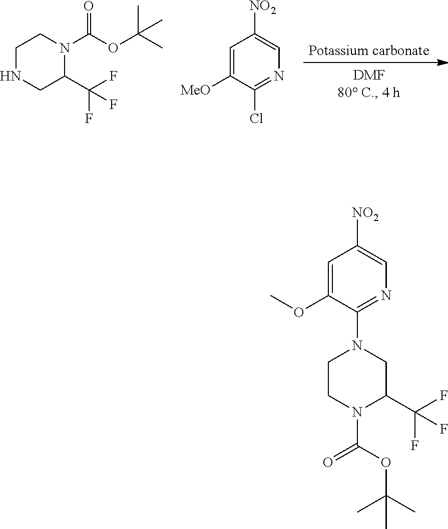 [SMILES: CC(C)(C)OC(=O)N1CCNCC1C(F)(F)F ; COc1cc([N+](=O)[O-])cnc1Cl ; "DMF80° C., 4 h" ; "Potassium carbonate" ; COc1cc([N+](=O)[O-])cnc1N1CCN(C(=O)OC(C)(C)C)C(C(F)(F)F)C1]

The title compound was prepared according to the coupling general procedure G starting from tert-butyl 2-(trifluoromethyl)piperazine-1-carboxylate and 2-chloro-3-methoxy-5-nitropyridine. MS (m/z): [M+H]⁺−407.3.

### Step 2—Preparation of tert-butyl 4-(5-amino-3-methoxypyridin-2-yl)-2-(trifluoromethyl)piperazine-1-carboxylate

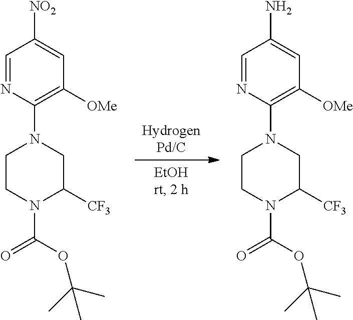 [SMILES: COc1cc([N+](=O)[O-])cnc1N1CCN(C(=O)OC(C)(C)C)C(C(F)(F)F)C1 ; "EtOHrt, 2 h" ; "HydrogenPd/C" ; COc1cc(N)cnc1N1CCN(C(=O)OC(C)(C)C)C(C(F)(F)F)C1]

The title compound was prepared according to the coupling general procedure H starting from tert-butyl 4-(3-methoxy-5-nitropyridin-2-yl)-2-(trifluoromethyl)piperazine-1-carboxylate. MS (m/z): [M+H]⁺−377.4.

### Step 3—Preparation of tert-butyl 4-(5-((4-((6,7-difluoroquinolin-3-yl)amino)pyrimidin-2-yl)amino)-3-methoxypyridin-2-yl)-2-(trifluoromethyl)piperazine-1-carboxylate

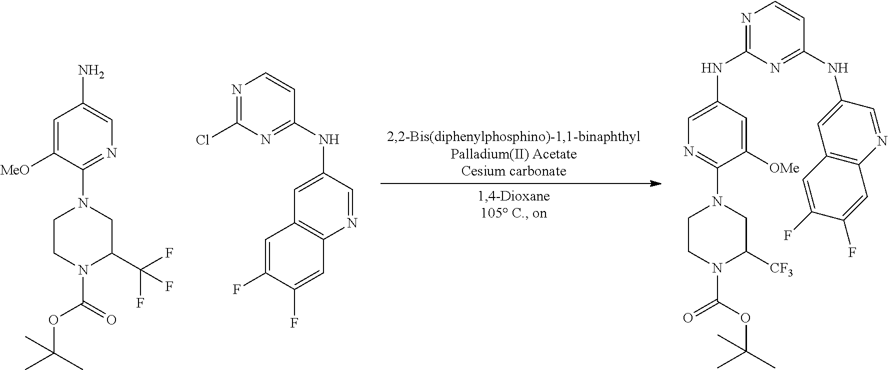 [SMILES: COc1cc(N)cnc1N1CCN(C(=O)OC(C)(C)C)C(C(F)(F)F)C1 ; Fc1cc2cc(Nc3ccnc(Cl)n3)cnc2cc1F ; "1,4-Dioxane105° C., on" ; "2,2-Bis(diphenylphosphino)-1,1-binaphthylPalladium(II) AcetateCesium carbonate" ; COc1cc(Nc2nccc(Nc3cnc4cc(F)c(F)cc4c3)n2)cnc1N1CCN(C(=O)OC(C)(C)C)C(C(F)(F)F)C1]

The title compound was prepared according to the coupling general procedure F starting from tert-butyl 4-(5-amino-3-methoxypyridin-2-yl)-2-(trifluoromethyl)piperazine-1-carboxylate and N-(2-chloropyrimidin-4-yl)-6,7-difluoroquinolin-3-amine. MS (m/z): [M+Boc]⁺−633.5.

### Step 4—Preparation of N4-(6,7-difluoroquinolin-3-yl)-N2-(5-methoxy-6-(3-(trifluoromethyl)piperazin-1-yl)pyridin-3-yl)pyrimidine-2,4-diamine

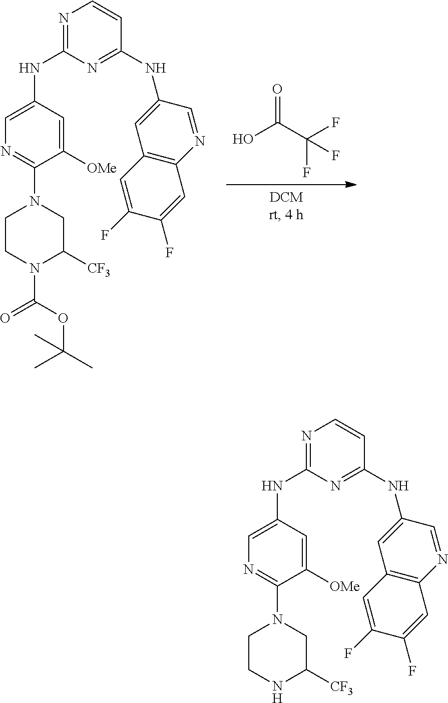

A solution of tert-butyl 4-(5-((4-((6,7-difluoroquinolin-3-yl)amino)pyrimidin-2-yl)amino)-3-methoxypyridin-2-yl)-2-(trifluoromethyl)piperazine-1-carboxylate (60 mg, 94.8 umol) in DCM (1 mL) was treated with TFA (147 uL) for 4 h at room temperature. The solvent was removed under vacuum and the crude product was dissolved in DMSO (1 mL) and was purified by HPLC Prep-MS eluting with a solvent gradient of 35-55% MeCN/H2O (0.1% of 10 mM ammonium bicarbonate buffer). The clean fractions were combined and lyophilized to afford the title product (10.0 mg, 20%) as a white solid. MS (m/z): [M+H]⁺−533.5. ¹H NMR (500 MHz, DMSO) δ (ppm): 9.99 (d, J=13.5 Hz, 1H), 9.17 (d, J=20.9 Hz, 1H), 8.99 (s, 1H), 8.93 (s, 1H), 8.55 (s, 1H), 8.22-8.20 (m, 1H), 8.12-8.10 (m, 1H), 7.96-7.89 (m, 1H), 7.73 (s, 1H), 7.56 (s, 1H), 6.33 (m, 1H), 3.76 (d, J=11.6 Hz, 1H), 3.67 (d, J=11.2 Hz, 3H), 3.51 (s, 1H), 3.00-2.94 (m, 1H), 2.88-2.72 (m, 3H).

### Example 335: Preparation of 3-((dimethylamino)methyl)-2,3-dihydro-[1,4]dioxino[2,3-b]pyridin-7-amine (Intermediate 128)

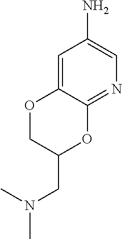

### Step 1: 5-bromo-2-chloro-3-(oxiran-2-ylmethoxy)pyridine

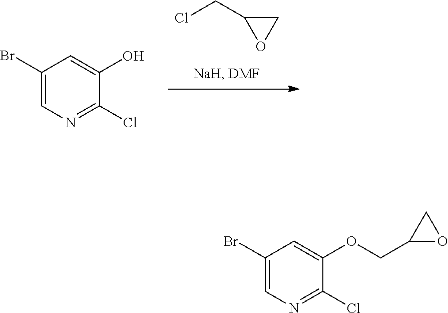

To a stirred suspension of sodium hydride 60% in dispersion in mineral oil (461 mg, 11.5 mmol) in DMF (20 mL) at rt was added 2-Chloro-5-methylpyridin-3-ol (2.00 g, 9.60 mmol) in 3 portions. After 45 min, (+)-epichlorohydrin (8.97 g, 96.0 mmol) was added via syringe and the mixture was heated at 60° C. overnight. After cooling to room temperature the mixture was poured into cold water and extracted with EtOAcOAc (50 mL×3). The organic layer was washed with brine and dried over MgSO4, evaporated to afford 5-bromo-2-chloro-3-(oxiran-2-ylmethoxy)pyridine (2.50 g, 89%) and used in the next step without further purification. MS (m/z): [M+H]⁺: 264.0, 266.0.

### Step 2: 1-((5-bromo-2-chloropyridin-3-yl)oxy)-3-(dimethylamino)propan-2-ol

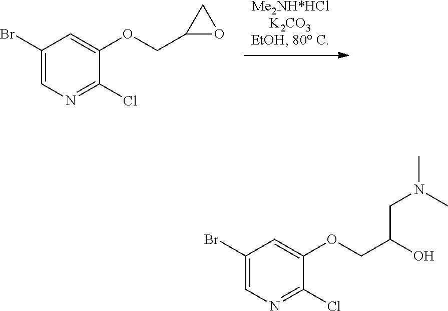 [SMILES: Clc1ncc(Br)cc1OCC1CO1 ; "Me2NH*HClK2CO3EtOH, 80° C." ; CN(C)CC(O)COc1cc(Br)cnc1Cl]

To a suspension of potassium carbonate (1.32 g, 9.36 mmol) in EtOH (40.0 mL) was added dimethylamine hydrochloride (728 mg, 8.93 mmol) and the solution was stirred for 30 min at rt. To the reaction mixture was added 5-bromo-2-chloro-3-(oxiran-2-ylmethoxy)pyridine (1.25 g, 4.25 mmol) in EtOH (20.00 mL) and the resulting solution was stirred at reflux for 2 h. The solvent was evaporated, residue was diluted with water, extracted with EtOAc (50 mL×3), dried over MgSO₄, filtered and the solvent was removed in vacuo to give the 1-((5-bromo-2-chloropyridin-3-yl)oxy)-3-(dimethylamino)propan-2-ol (1.10 g, 67%) which was used in next step without further purification. MS (m/z): [M+H]⁺: 309.0, 311.0.

### Step 3: 1-(7-bromo-2,3-dihydro-[1,4]dioxino[2,3-b]pyridin-3-yl)-N,N-dimethylmethanamine

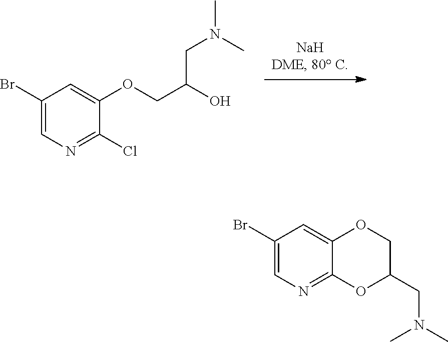 [SMILES: CN(C)CC(O)COc1cc(Br)cnc1Cl ; "NaHDME, 80° C." ; CN(C)CC1COc2cc(Br)cnc2O1]

To a suspension of sodium hydride 60% in dispersion in mineral oil (388 mg, 9.69 mmol) in dry dimethoxyethane (125.0 mL) was added a solution of 1-((5-bromo-2-chloropyridin-3-yl)oxy)-3-(dimethylamino)propan-2-ol (1.50 g, 4.85 mmol) in dimethoxyethane. The resulting mixture was heated at 80° C. for 72 h. After cooling to room temperature, the reaction was quenched with water and extracted with EtOAc. The organic layer was dried over MgSO4, filtered and concentrated in vacuo. Purification by flash column chromatography (eluent: i-PrOH/CH₂Cl₂, gradient: 0.2:9.8 to 1.5:8.5) to afford 1-(7-bromo-2,3-dihydro-[1,4]dioxino[2,3-b]pyridin-3-yl)-N,N-dimethylmethanamine (680 mg, 26%). MS (m/z): [M+H]⁺: 273.0, 275.0.

### Step 4: tert-butyl (3-((dimethylamino)methyl)-2,3-dihydro-[1,4]dioxino[2,3-b]pyridin-7-yl)carbamate

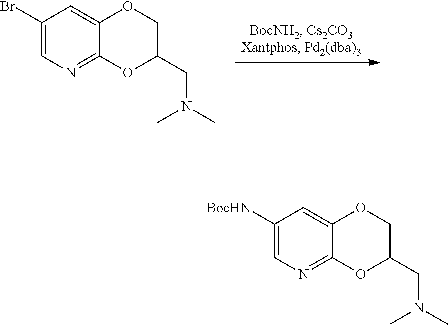 [SMILES: CN(C)CC1COc2cc(Br)cnc2O1 ; "BocNH2, Cs2CO3Xantphos, Pd2(dba)3" ; CN(C)CC1COc2cc(NC(=O)OC(C)(C)C)cnc2O1]

A suspension of 1-(7-bromo-2,3-dihydro-[1,4]dioxino[2,3-b]pyridin-3-yl)-N,N-dimethylmethanamine (580 mg, 2.12 mmol), tert-Butyl carbamate (305 mg, 2.55 mmol), Tris(dibenzylideneacetone)-dipalladium(0) (205 mg, 212 umol), 4,4-bis(diphenylphosphino)-9,9-dimethylxanthene (188 mg, 319 umol) and cesium carbonate (1.06 g, 3.19 mmol) in anhydrous dioxane (4.26 mL) was degassed and purged with N₂ (3×). The reaction mixture was then heated at 100° C. for 16 h. The reaction was cooled to rt, water (15 mL) was added, then extracted with EtOAc (50 mL×3). The combined organic layers were washed brine (15 mL), dried over anhydrous Na₂SO₄ and concentrated. The residue was purified by flash column chromatography (0% to 10% MeOH in DCM) to give tert-butyl (3-((dimethylamino)methyl)-2,3-dihydro-[1,4]dioxino[2,3-b]pyridin-7-yl)carbamate (420 mg, 64%) as a yellow oil. MS (m/z): [M+H]⁺: 310.4.

### Step 5: 3-((dimethylamino)methyl)-2,3-dihydro-[1,4]dioxino[2,3-b]pyridin-7-amine

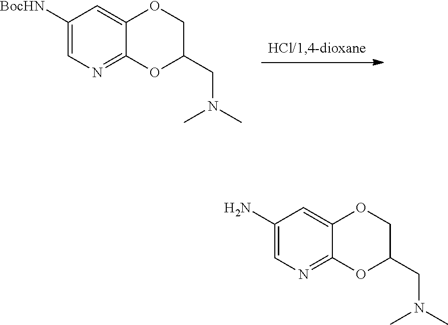 [SMILES: CN(C)CC1COc2cc(NC(=O)OC(C)(C)C)cnc2O1 ; "HCl/1,4-dioxane" ; CN(C)CC1COc2cc(N)cnc2O1]

The title compound was prepared according to the general procedure AB starting from tert-butyl (3-((dimethylamino)methyl)-2,3-dihydro-[1,4]dioxino[2,3-b]pyridin-7-yl)carbamate. MS (m/z): [M+H]⁺: 210.2

### Example 336: Preparation of Compound 177

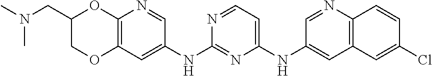 [SMILES: CN(C)CC1COc2cc(Nc3nccc(Nc4cnc5ccc(Cl)cc5c4)n3)cnc2O1]

Compound 177 was prepared from the corresponding intermediate according to general procedure E. MS (m/z): [M+H]⁺ 464.2. H NMR (500 MHz, DMSO) δ (ppm): 10.07 (s, 1H), 9.31 (s, 1H), 8.98 (s, 1H), 8.92 (s, 1H), 8.12 (d, J=5.7 Hz, 1H), 7.99 (s, 1H), 7.94 (d, J=8.9 Hz, 2H), 7.81 (s, 1H), 7.59 (dd, J=8.9, 2.4 Hz, 1H), 6.37 (d, J=5.7 Hz, 1H), 4.52-4.43 (m, 1H), 4.32 (dd, J=11.5, 2.3 Hz, 1H), 3.98 (dd, J=11.5, 7.5 Hz, 1H), 2.56 (dd, J=6.1, 1.9 Hz, 2H), 2.25 (s, 6H).

### Example 337: Preparation of Compound 178

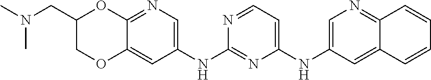 [SMILES: CN(C)CC1COc2cc(Nc3nccc(Nc4cnc5ccccc5c4)n3)cnc2O1]

Compound 178 was prepared from the corresponding intermediate according to general procedure E. MS (m/z): [M+H]⁺ 430.4. H NMR (500 MHz, DMSO) δ 9.91 (s, 1H), 9.27 (s, 1H), 8.92 (s, 2H), 8.10 (d, J=5.7 Hz, 1H), 8.01 (d, J=2.2 Hz, 1H), 7.93 (d, J=8.1 Hz, 1H), 7.84 (d, J=7.5 Hz, 1H), 7.77 (s, 1H), 7.65-7.50 (m, 2H), 6.35 (d, J=5.7 Hz, 1H), 4.48-4.42 (m, 1H), 4.27 (dd, J=11.5, 2.2 Hz, 1H), 3.96 (dd, J=11.5, 7.3 Hz, 1H), 2.55 (dd, J=6.1, 2.9 Hz, 2H), 2.24 (s, 6H).

### Example 338: Preparation of Compound 179

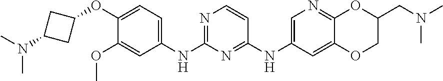

Compound 179 was prepared from the corresponding intermediate according to general procedure C. MS (m/z): [M+H]⁺ 522.5. H NMR (500 MHz, DMSO) δ 9.32 (s, 1H), 8.98 (s, 1H), 8.26 (s, 1H, HCOOH), 8.01-7.94 (m, 2H), 7.83 (d, J=2.1 Hz, 1H), 7.29 (s, 1H), 7.22 (dd, J=8.7, 2.1 Hz, 1H), 6.71 (d, J=8.8 Hz, 1H), 6.13 (d, J=5.7 Hz, 1H), 4.51-4.42 (m, 1H), 4.34-4.25 (m, 2H), 3.99 (dd, J=11.5, 7.3 Hz, 1H), 3.66 (s, 3H), 2.62-2.53 (m, 4H), 2.45-2.36 (m, 1H), 2.25 (s, 6H), 2.11 (s, 6H), 1.89-1.83 (m, 2H) (formate salt).

### Example 339: Preparation of cis-(1,2)-2-((5-amino-3-fluoropyridin-2-yl)oxy)cyclohexanol (Intermediate 129)

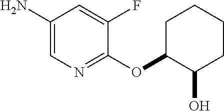

### Step 1: cis-(1,2)-2-((3-fluoro-5-nitropyridin-2-yl)oxy)cyclohexanol

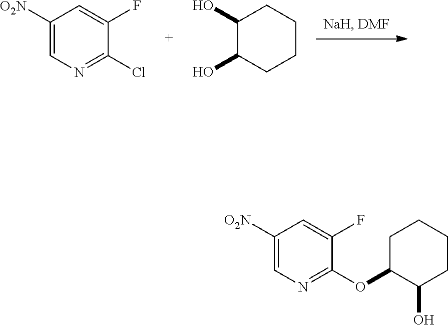

The title compound was prepared according to the general procedure AD starting from 2-chloro-3-fluoro-5-nitropyridine and cis-cyclohexane-1,2-diol. MS (m/z): [M+H]⁺: 257.2.

### Step 2: cis-(1,2)-2-((5-amino-3-fluoropyridin-2-yl)oxy)cyclohexanol

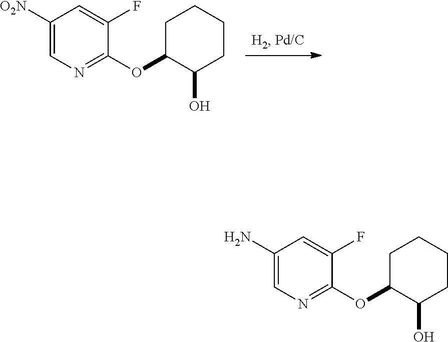

The title compound was prepared according to the general procedure H starting from cis-(1,2)-2-((3-fluoro-5-nitropyridin-2-yl)oxy)cyclohexanol. MS (m/z): [M+H]⁺: 227.2.

### Example 340: Preparation of Compound 180

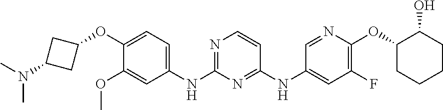 [SMILES: COc1cc(Nc2nccc(Nc3cnc(O[C@H]4CCCC[C@H]4O)c(F)c3)n2)ccc1O[C@H]1C[C@@H](N(C)C)C1]

Compound 180 was prepared from the corresponding intermediate according to general procedure C. MS (m/z): [M+H]⁺ 539.5. H NMR (500 MHz, DMSO) δ 9.43 (s, 1H), 9.02 (s, 1H), 8.22 (s, 1H), 8.15 (d, J=12.5 Hz, 1H), 8.00 (d, J=5.7 Hz, 1H), 7.28 (s, 1H), 7.22 (d, J=7.3 Hz, 1H), 6.71 (d, J=8.7 Hz, 1H), 6.15 (d, J=5.7 Hz, 1H), 5.09 (d, J=8.1 Hz, 1H), 4.42-4.18 (m, J=7.1 Hz, 1H), 3.88 (d, J=7.4 Hz, 1H), 3.68 (s, 3H), 2.65-2.52 (m, 2H), 2.38-2.24 (m, 1H), 2.06 (s, 6H), 1.96-1.86 (m, 1H), 1.85-1.77 (m, 2H), 1.77-1.69 (m, 1H), 1.67-1.59 (m, 1H), 1.59-1.50 (m, 3H), 1.41-1.25 (m, 2H).

### Example 341: Preparation of cis-(5a,9a)-5a,6,7,8,9,9a-hexahydrobenzo[5,6][1,4]dioxino[2,3-b]pyridin-3-amine (Intermediate 130)

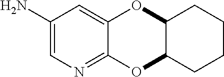

### Step 1: cis-(5a,9a)-3-nitro-5a,6,7,8,9,9a-hexahydrobenzo[5,6][1,4]dioxino[2,3-b]pyridine

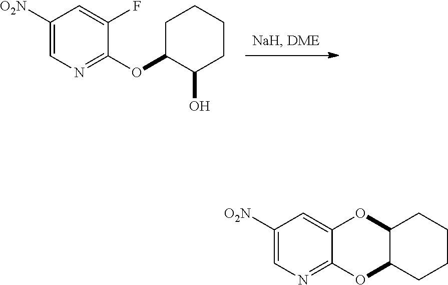

The title compound was prepared according to the general procedure AE starting from cis-(1,2)-2-((5-amino-3-fluoropyridin-2-yl)oxy)cyclohexanol. MS (m/z): [M+H]⁺: 237.3.

### Step 2: cis-(5a,9a)-3-nitro-5a,6,7,8,9,9a-hexahydrobenzo[5,6][1,4]dioxino[2,3-b]pyridine

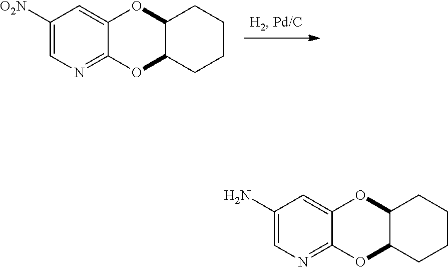

The title compound was prepared according to the general procedure H starting from cis-(5a,9a)-3-nitro-5a,6,7,8,9,9a-hexahydrobenzo[5,6][1,4]dioxino[2,3-b]pyridine. MS (m/z): [M+H]⁺: 207.1.

### Example 342: Preparation of Compound 190

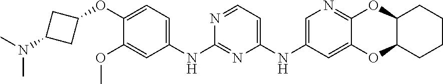 [SMILES: COc1cc(Nc2nccc(Nc3cnc4c(c3)O[C@@H]3CCCC[C@@H]3O4)n2)ccc1O[C@H]1C[C@@H](N(C)C)C1]

Compound 190 was prepared from the corresponding intermediate according to general procedure C. MS (m/z): [M+H]⁺ 519.6. H NMR (500 MHz, DMSO) δ 9.28 (s, 1H), 8.96 (s, 1H), 7.96 (d, J=5.7 Hz, 1H), 7.92 (s, 1H), 7.80 (d, J=2.1 Hz, 1H), 7.28 (s, 1H), 7.18 (d, J=7.5 Hz, 1H), 6.70 (s, 1H), 6.12 (d, J=5.7 Hz, 1H), 4.45-4.35 (m, 1H), 4.35-4.21 (m, 2H), 3.65 (s, 3H), 2.58-2.51 (m, 2H), 2.36-2.21 (m, 1H), 2.04 (s, 6H), 1.84-1.69 (m, 6H), 1.67-1.53 (m, 2H), 1.48-1.34 (m, 2H).

### Example 343: Preparation of trans-(5a,9a)-5a,6,7,8,9,9a-hexahydrobenzo[5,6][1,4]dioxino[2,3-b]pyridin-3-amine (Intermediate 131)

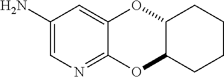

### Step 1: trans-(1,2)-2-((3-fluoro-5-nitropyridin-2-yl)oxy)cyclohexanol

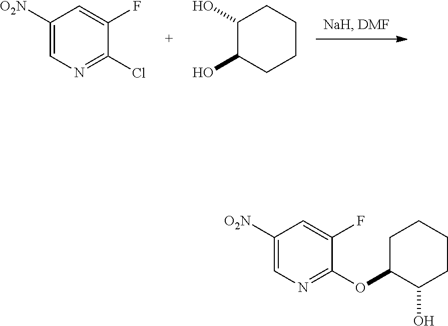

The title compound was prepared according to the general procedure AD starting from trans-(1,2)-cyclohexane-1,2-diol. MS (m/z): [M+H]⁺: 257.1.

### Step 2: trans-(5a,9a)-3-nitro-5a,6,7,8,9,9a-hexahydrobenzo[5,6][1,4]dioxino[2,3-b]pyridine

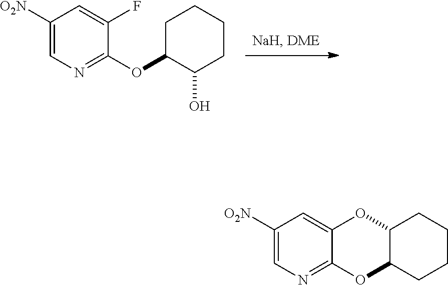

The title compound was prepared according to the general procedure AE starting from trans-(1,2)-2-((3-fluoro-5-nitropyridin-2-yl)oxy)cyclohexanol. MS (m/z): [M+H]⁺: 237.0.

### Step 3: trans-(5a,9a)-5a,6,7,8,9,9a-hexahydrobenzo[5,6][1,4]dioxino[2,3-b]pyridin-3-amine

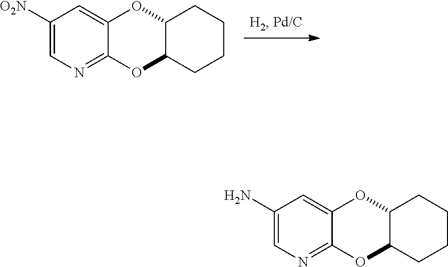

The title compound was prepared according to the general procedure H starting from trans-(5a,9a)-3-nitro-5a,6,7,8,9,9a-hexahydrobenzo[5,6][1,4]dioxino[2,3-b]pyridine. MS (m/z): [M+H]⁺: 207.1.

### Example 344: Preparation of Compound 191

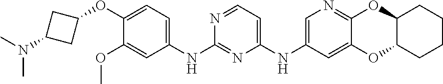 [SMILES: COc1cc(Nc2nccc(Nc3cnc4c(c3)O[C@H]3CCCC[C@@H]3O4)n2)ccc1O[C@H]1C[C@@H](N(C)C)C1]

Compound 191 was prepared from the corresponding intermediate according to general procedure C. MS (m/z): [M+H]⁺ 519.5. H NMR (500 MHz, DMSO) δ 9.28 (s, 1H), 8.97 (s, 1H), 7.97 (d, J=5.7 Hz, 2H), 7.78 (d, J=2.3 Hz, 1H), 7.28 (s, 1H), 7.20 (d, J=8.6 Hz, 1H), 6.69 (d, J=8.7 Hz, 1H), 6.12 (d, J=5.7 Hz, 1H), 4.30 (p, J=7.3 Hz, 1H), 3.88 (ddd, J=11.2, 8.2, 4.6 Hz, 1H), 3.83-3.72 (m, 1H), 3.65 (s, 3H), 2.55 (dtd, J=9.3, 6.7, 2.8 Hz, 2H), 2.41-2.25 (m, 1H), 2.17 (d, J=10.3 Hz, 2H), 2.07 (s, 6H), 1.82 (ddd, J=16.4, 8.5, 2.8 Hz, 2H), 1.76 (d, J=6.3 Hz, 2H), 1.59-1.34 (m, 4H).

### Example 345: Preparation of trans-(5a,8a)-6,7,8,8a-tetrahydro-5aH-cyclopenta[5,6][1,4]dioxino[2,3-b]pyridin-3-amine (Intermediate 132)

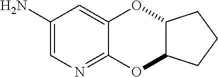

### Step 1: trans-(1S,2S)-2-((3-fluoro-5-nitropyridin-2-yl)oxy)cyclopentanol

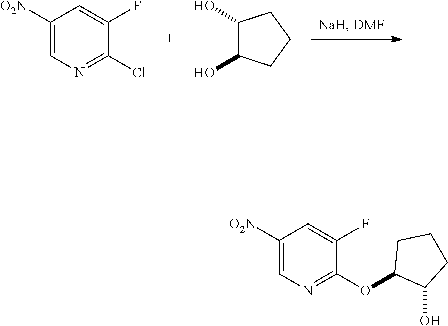

The title compound was prepared according to the general procedure AD starting from trans-(1,2)-cyclopentane-1,2-diol. MS (m/z): [M−H]−: 241.0.

### Step 2: trans-(5a,8a)-3-nitro-6,7,8,8a-tetrahydro-5aH-cyclopenta[5,6][1,4]dioxino[2,3-b]pyridine

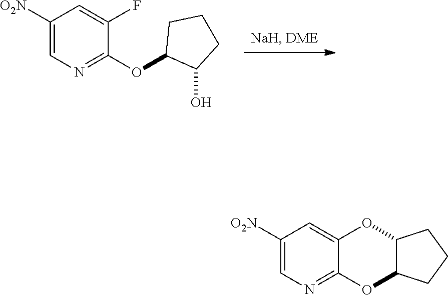

The title compound was prepared according to the general procedure AE starting from trans-(1,2)-2-((3-fluoro-5-nitropyridin-2-yl)oxy)cyclopentanol. MS (m/z): [M+H]⁺: 223.0.

### Step 3: trans-(5a,8a)-6,7,8,8a-tetrahydro-5aH-cyclopenta[5,6][1,4]dioxino[2,3-b]pyridin-3-amine

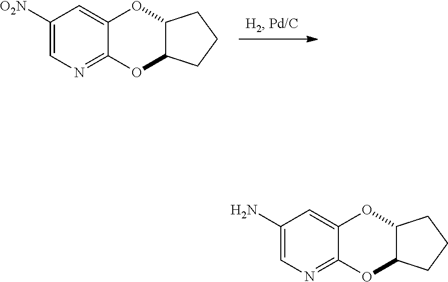

The title compound was prepared according to the general procedure H starting from trans-(5a,8a)-3-nitro-6,7,8,8a-tetrahydro-5aH-cyclopenta[5,6][1,4]dioxino[2,3-b]pyridine MS (m/z): [M+H]⁺: 193.2.

### Example 346: Preparation of Compound 192

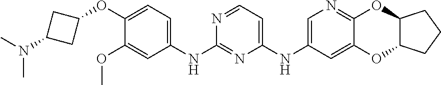 [SMILES: COc1cc(Nc2nccc(Nc3cnc4c(c3)O[C@H]3CCC[C@@H]3O4)n2)ccc1O[C@H]1C[C@@H](N(C)C)C1]

Compound 192 was prepared from the corresponding intermediate according to general procedure C. MS (m/z): [M+H]⁺ 505.5. ¹H NMR (500 MHz, DMSO) δ 9.35 (s, 1H), 8.98 (s, 1H), 8.27 (s, 1H, HCOOH), 8.04 (d, J=1.6 Hz, 1H), 7.97 (d, J=5.7 Hz, 1H), 7.86 (d, J=2.2 Hz, 1H), 7.29 (d, J=1.8 Hz, 1H), 7.22 (dd, J=8.7, 2.1 Hz, 1H), 6.70 (d, J=8.7 Hz, 1H), 6.13 (d, J=5.7 Hz, 1H), 4.35-4.25 (m, 1H), 4.13 (dt, J=10.8, 7.8 Hz, 1H), 4.04 (dt, J=10.8, 7.8 Hz, 1H), 3.66 (s, 3H), 2.54 (tdd, J=6.6, 5.8, 2.4 Hz, 2H), 2.35-2.27 (m, 1H), 2.20-2.07 (m, 2H), 2.10-2.01 (m, 6H), 1.88 (ddd, J=15.5, 10.6, 5.1 Hz, 2H), 1.81 (ddd, J=16.3, 8.5, 2.8 Hz, 2H), 1.68 (tt, J=20.7, 10.5 Hz, 2H).

### Example 347: Preparation of Compound 193

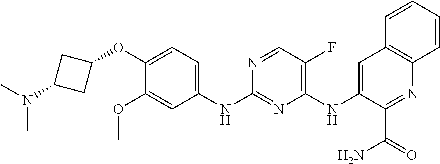 [SMILES: COc1cc(Nc2ncc(F)c(Nc3cc4ccccc4nc3C(N)=O)n2)ccc1O[C@H]1C[C@@H](N(C)C)C1]

### Step 1—Preparation of 3-((2-chloro-5-fluoropyrimidin-4-yl)amino)quinoline-2-carboxamide

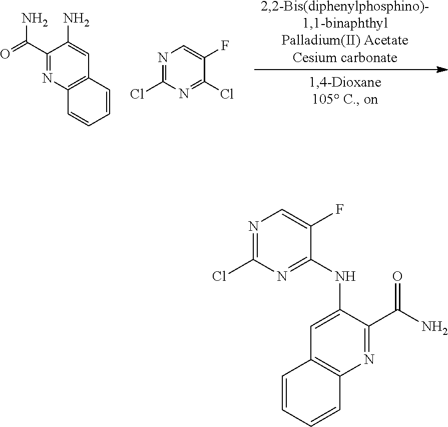

The title compound was prepared according to the coupling general procedure F starting from 3-aminoquinoline-2-carboxamide and 2,4-dichloro-5-fluoropyrimidine. MS (m/z): [M−H]⁻−316.3, 318.1.

### Step 2—Preparation of N4-(6,7-difluoroquinolin-3-yl)-N2-(4-((1s,3s)-3-(dimethylamino)cyclobutoxy)-3-methoxyphenyl)-5-fluoropyrimidine-2,4-diamine

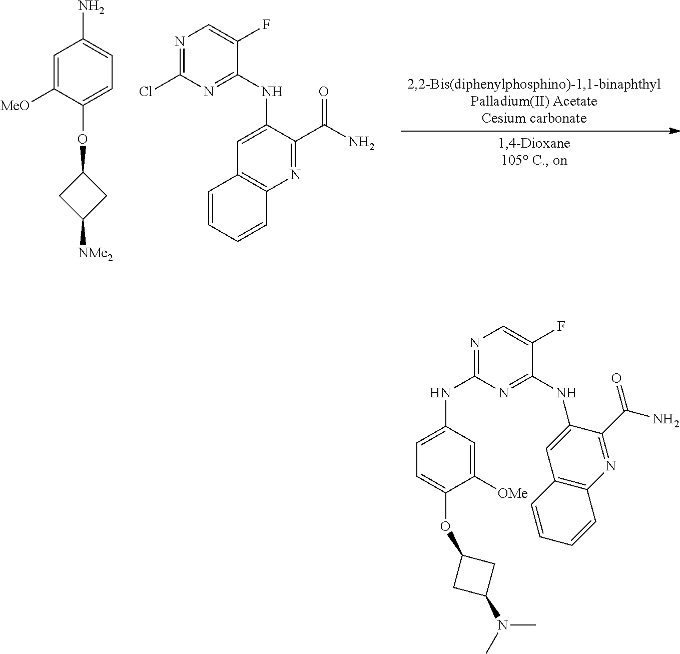

The title compound was prepared according to the coupling general procedure F starting from 4-((1s,3s)-3-(dimethylamino)cyclobutoxy)-3-methoxyaniline and 3-((2-chloro-5-fluoropyrimidin-4-yl)amino) quinoline-2-carboxamide. MS (m/z): [M+H]⁺−518.4. ¹H NMR (500 MHz, DMSO) δ (ppm): 12.49 (s, 1H), 9.77 (s, 1H), 9.27 (s, 1H), 8.84 (s, 1H), 8.26-8.23 (m, 2H), 8.05 (d, J=8.4 Hz, 1H), 7.82 (s, 1H), 7.75-7.69 (m, 2H), 7.30-7.23 (m, 2H), 6.84 (d, J=8.6 Hz, 1H), 4.48-4.39 (m, 1H), 3.70 (s, 3H), 3.31-3.30 (m, 1H), 2.78 (s, 2H), 2.55-2.52 (m, 6H), 2.29-2.16 (m, 2H).

### Example 348: Preparation of Compound 194

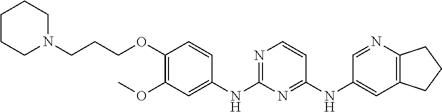 [SMILES: COc1cc(Nc2nccc(Nc3cnc4c(c3)CCC4)n2)ccc1OCCCN1CCCCC1]

Compound 194 was prepared from the corresponding intermediate according to general procedure C. MS (m/z): [M+H]⁺: 475.6. H NMR (400 MHz, DMSO) δ 9.29 (s, 1H), 8.89 (s, 1H), 8.36 (s, 1H), 8.04 (d, J=2.1 Hz, 1H), 7.94 (d, J=5.7 Hz, 1H), 7.22 (d, J=2.1 Hz, 1H), 7.11 (d, J=8.6 Hz, 1H), 6.78 (d, J=8.7 Hz, 1H), 6.11 (d, J=5.7 Hz, 1H), 3.88 (t, J=6.4 Hz, 2H), 3.58 (s, 3H), 2.79 (t, J=7.6 Hz, 4H), 2.35-2.25 (m, 6H), 2.01 (q, J=7.2 Hz, 2H), 1.77 (q, J=7.2 Hz, 2H), 1.47-1.40 (m, 4H), 1.35-1.30 (m, 2H).

### Example 349: Preparation of Compound 195

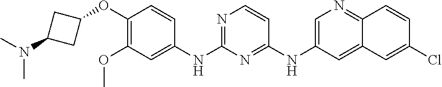 [SMILES: COc1cc(Nc2nccc(Nc3cnc4ccc(Cl)cc4c3)n2)ccc1O[C@H]1C[C@H](N(C)C)C1]

Compound 195 was prepared according to general procedure C. MS (m/z): [M+H]⁺: 491.6, 493.5. ¹H NMR (400 MHz, DMSO) δ 9.93 (s, 1H), 9.06 (s, 1H), 9.01 (s, 1H), 8.94-8.90 (m, 1H), 8.13-8.09 (m, 1H), 7.95-7.92 (m, 1H), 7.81 (s, 1H), 7.61-7.57 (m, 1H), 7.27 (s, 1H), 7.22 (d, J=8.1 Hz, 1H), 6.67 (s, 1H), 6.30 (s, 1H), 4.69 (s, 1H), 3.68 (s, 3H), 2.84-2.80 (m, 1H), 2.32-2.28 (m, 2H), 2.19-2.00 (m, 8H).

### Example 350: Preparation of Compound 196

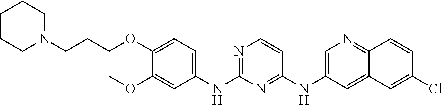 [SMILES: COc1cc(Nc2nccc(Nc3cnc4ccc(Cl)cc4c3)n2)ccc1OCCCN1CCCCC1]

Compound 196 was prepared according to general procedure C. MS (m/z): [M+H]⁺: 519.6. ¹H NMR (500 MHz, DMSO) δ 9.94 (s, 1H), 9.08 (s, 1H), 9.05 (s, 1H), 8.92 (d, J=2.1 Hz, 1H), 8.11 (d, J=5.6 Hz, 1H), 7.94 (d, J=8.9 Hz, 1H), 7.82 (s, 1H), 7.59 (dd, J=8.9, 2.4 Hz, 1H), 7.25 (d, J=8.6 Hz, 2H), 6.91 (d, J=8.6 Hz, 1H), 6.32 (d, J=5.7 Hz, 1H), 3.99 (t, J=6.4 Hz, 2H), 3.68 (s, 3H), 2.40 (t, J=7.2 Hz, 2H), 2.33 (s, 4H), 1.90-1.82 (m, 2H), 1.52-1.45 (m, 4H), 1.38 (d, J=4.9 Hz, 2H).

### Example 351: Preparation of Compound 197

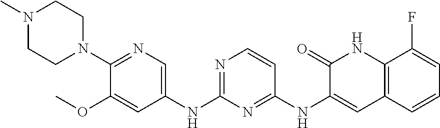 [SMILES: COc1cc(Nc2nccc(Nc3cc4cccc(F)c4[nH]c3=O)n2)cnc1N1CCN(C)CC1]

Compound 197 was prepared according to general procedure C. MS (m/z): [M+H]⁺: 477.5. ¹H NMR (500 MHz, DMSO) δ 12.29 (s, 1H), 9.16 (s, 1H), 9.11 (s, 1H), 8.96 (s, 1H), 8.14 (d, J=1.7 Hz, 1H), 8.08 (d, J=5.7 Hz, 1H), 7.50 (s, 1H), 7.31-7.23 (m, 2H), 7.12 (s, 1H), 6.79 (d, J=5.7 Hz, 1H), 3.72 (s, 3H), 3.29 (s, 4H), 2.47 (s, 4H), 2.24 (s, 3H).

### Example 352: Preparation of Compound 198

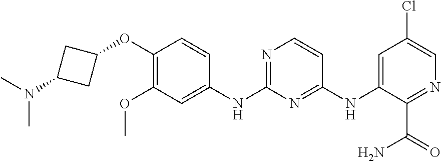 [SMILES: COc1cc(Nc2nccc(Nc3cc(Cl)cnc3C(N)=O)n2)ccc1O[C@H]1C[C@@H](N(C)C)C1]

Compound 198 was prepared according to general procedure C. MS (m/z): [M+H]⁺: 484.3. ¹H NMR (400 MHz, DMSO) δ 11.97 (s, 1H), 9.29 (s, 1H), 9.19 (s, 1H), 8.41 (s, 1H), 8.16 (d, J=2.1 Hz, 1H), 8.09 (d, J=5.6 Hz, 1H), 7.97 (s, 1H), 7.10 (d, J=8.8 Hz, 2H), 6.74 (d, J=8.4 Hz, 1H), 6.22 (d, J=5.6 Hz, 1H), 4.34-4.23 (m, 1H), 3.65 (s, 3H), 2.52 (dd, J=9.1, 2.6 Hz, 1H), 2.33-2.22 (m, 2H), 2.00 (s, 6H), 1.77 (dd, J=19.3, 8.3 Hz, 2H).

### Example 353: Preparation of Compound 199

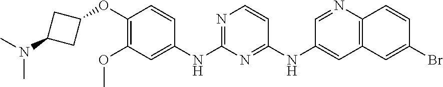 [SMILES: COc1cc(Nc2nccc(Nc3cnc4ccc(Br)cc4c3)n2)ccc1O[C@H]1C[C@H](N(C)C)C1]

Compound 199 was prepared according to general procedure C. MS (m/z): [M+H]⁺: 535.6, 537.3. ¹H NMR (500 MHz, DMSO) δ 9.93 (s, 1H), 9.06 (s, 1H), 9.04-9.01 (m, 1H), 8.94-8.92 (m, 1H), 8.11-8.08 (m, 1H), 8.01-7.98 (m, 1H), 7.88-7.84 (m, 1H), 7.71-7.68 (m, 1H), 7.28-7.21 (m, 2H), 6.68 (d, J=8.7 Hz, 1H), 6.31 (d, J=5.7 Hz, 1H), 4.69 (s, 1H), 3.68 (s, 3H), 2.84-2.80 (m, 1H), 2.32-2.27 (m, 2H), 2.18-2.13 (m, 2H), 2.06 (s, 6H).

### Example 354: Preparation of Compound 200

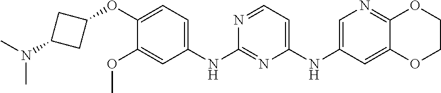 [SMILES: COc1cc(Nc2nccc(Nc3cnc4c(c3)OCCO4)n2)ccc1O[C@H]1C[C@@H](N(C)C)C1]

Compound 200 was prepared according to general procedure E. MS (m/z): [M+H]⁺: 465.6. ¹H NMR (400 MHz, DMSO): δ 9.23 (s, 1H), 8.90 (s, 1H), 7.94 (d, J=2.4 Hz, 1H), 7.92 (d, J=5.7 Hz, 1H), 7.72 (d, J=2.3 Hz, 1H), 7.25 (d, J=2.3 Hz, 1H), 7.15 (dd, J=8.5, 2.4 Hz, 1H), 6.66 (d, J=8.7 Hz, 1H), 6.07 (d, J=5.8 Hz, 1H), 4.32 (dd, J=5.2, 3.0 Hz, 2H), 4.29-4.21 (m, 1H), 4.19 (dd, J=5.1, 3.0 Hz, 2H), 3.61 (s, 3H), 2.54-2.47 (m, 2H), 2.24 (d, J=8.2 Hz, 1H), 1.99 (s, 6H), 1.75 (ddd, J=16.1, 8.5, 2.2 Hz, 2H).

### Example 355: Preparation of Compound 201

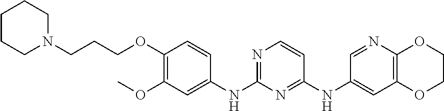 [SMILES: COc1cc(Nc2nccc(Nc3cnc4c(c3)OCCO4)n2)ccc1OCCCN1CCCCC1]

Compound 201 was prepared according to general procedure C. MS (m/z): [M+H]⁺: 493.5. ¹H NMR (500 MHz, DMSO) δ 9.23 (s, 1H), 8.92 (s, 1H), 8.02-7.87 (m, 2H), 7.73 (d, J=2.0 Hz, 1H), 7.27 (s, 1H), 7.18 (dd, J=8.5, 1.9 Hz, 1H), 6.78 (d, J=8.8 Hz, 1H), 6.08 (d, J=5.6 Hz, 1H), 4.32 (dd, J=4.7, 2.9 Hz, 2H), 4.21 (dd, J=4.9, 2.3 Hz, 2H), 3.88 (t, J=6.3 Hz, 2H), 3.63 (s, 3H), 2.41-2.25 (m, 6H), 1.84-1.71 (m, 2H), 1.50-1.39 (m, 4H), 1.39-1.25 (m, 2H).

### Example 356: Preparation of Compound 202

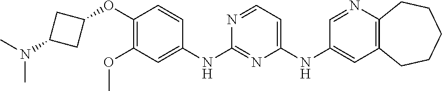 [SMILES: COc1cc(Nc2nccc(Nc3cnc4c(c3)CCCCC4)n2)ccc1O[C@H]1C[C@@H](N(C)C)C1]

Compound 202 was prepared according to general procedure C. MS (m/z): [M+H]⁺: 475.2 ¹H NMR (500 MHz, DMSO) δ (ppm): 9.33 (s, 1H), 8.95 (s, 1H), 8.44 (s, 1H), 7.99 (d, J=5.7 Hz, 1H), 7.95 (d, J=2.4 Hz, 1H), 7.27 (s, 1H), 7.17 (s, 1H), 6.72 (d, J=8.7 Hz, 1H), 6.17 (d, J=5.7 Hz, 1H), 4.32-4.29 (m, 1H), 3.65 (s, 3H), 2.95-2.90 (m, 2H), 2.71-2.67 (m, 2H), 2.56-2.54 (m, 1H), 2.34-2.27 (m, 2H), 2.10-2.03 (m, 6H), 1.86-1.77 (m, 4H), 1.63-1.56 (m, 4H).

### Example 357: Preparation of Compound 203

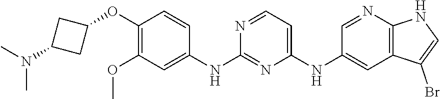 [SMILES: COc1cc(Nc2nccc(Nc3cnc4[nH]cc(Br)c4c3)n2)ccc1O[C@H]1C[C@@H](N(C)C)C1]

Compound 203 was prepared according to general procedure C. MS (m/z): [M+H]⁺: 524.4, 526.2. ¹H NMR (500 MHz, DMSO) δ 11.99 (s, 1H), 9.30 (s, 1H), 8.90 (s, 1H), 8.50 (s, 1H), 7.99-7.96 (m, 2H), 7.68 (s, 1H), 7.27 (s, 1H), 7.20-7.17 (m, 1H), 6.62 (d, J=8.7 Hz, 1H), 6.14 (d, J=5.7 Hz, 1H), 4.28-4.23 (m, 1H), 3.53 (s, 3H), 2.54-2.51 (m, 2H), 2.31-2.26 (m, 1H), 2.04 (s, 6H), 1.80-1.75 (m, 2H).

### Example 358: Preparation of Compound 204

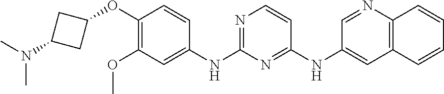 [SMILES: COc1cc(Nc2nccc(Nc3cnc4ccccc4c3)n2)ccc1O[C@H]1C[C@@H](N(C)C)C1]

Compound 204 was prepared according to general procedure E. MS (m/z): [M+H]⁺: 457.4. ¹H NMR (500 MHz, DMSO): δ 9.82 (s, 1H), 9.07 (s, 1H), 8.96 (s, 1H), 8.92 (d, J=2.2 Hz, 1H), 8.08 (d, J=5.7 Hz, 1H), 7.92 (d, J=8.2 Hz, 1H), 7.74 (d, J=7.8 Hz, 1H), 7.64-7.57 (m, 1H), 7.53 (t, J=6.9 Hz, 1H), 7.27 (s, 1H), 7.22 (d, J=8.6 Hz, 1H), 6.76 (d, J=8.6 Hz, 1H), 6.30 (d, J=5.7 Hz, 1H), 4.34 (p, J=7.1 Hz, 1H), 3.63 (s, 3H), 2.57 (ddd, J=11.8, 9.3, 6.8 Hz, 2H), 2.36-2.27 (m, 1H), 2.06 (s, 6H), 1.84 (ddd, J=16.1, 8.4, 2.7 Hz, 2H).

### Example 359: Preparation of Compound 205

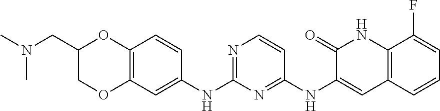 [SMILES: CN(C)CC1COc2cc(Nc3nccc(Nc4cc5cccc(F)c5[nH]c4=O)n3)ccc2O1]

Compound 205 was prepared according to general procedure C. MS (m/z): [M+H]⁺: 463.4. ¹H NMR (500 MHz, DMSO) δ 11.93 (s, 1H), 9.08 (d, J=23.5 Hz, 2H), 8.98 (s, 1H), 8.07 (d, J=5.7 Hz, 1H), 7.35 (s, 2H), 7.26-7.22 (m, 1H), 7.18-7.14 (m, 1H), 7.06 (d, J=8.8 Hz, 1H), 6.82-6.75 (m, 2H), 4.30-4.25 (m, 2H), 3.96-3.92 (m, 1H), 2.53-2.51 (m, 2H), 2.24 (s, 6H).

### Example 360: Preparation of Compound 206

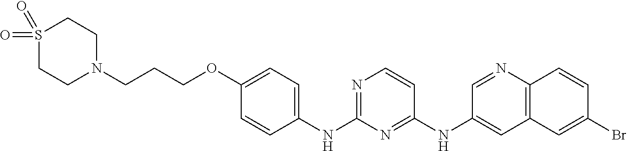 [SMILES: O=S1(=O)CCN(CCCOc2ccc(Nc3nccc(Nc4cnc5ccc(Br)cc5c4)n3)cc2)CC1]

Compound 206 was prepared according to general procedure C. MS (m/z): [M+H]⁺: 583.6, 585.6. ¹H NMR (500 MHz, DMSO) δ 9.93 (s, 1H), 9.08 (s, 1H), 9.03-8.99 (m, 1H), 8.90 (d, J=2.4 Hz, 1H), 8.09 (d, J=5.7 Hz, 1H), 7.98 (s, 1H), 7.86 (d, J=8.9 Hz, 1H), 7.70-7.68 (m, 1H), 7.56 (d, J=8.7 Hz, 2H), 6.91-6.89 (m, 2H), 6.30 (d, J=5.7 Hz, 1H), 4.02 (t, J=6.3 Hz, 2H), 3.10-3.06 (m, 4H), 2.92-2.89 (m, 4H), 2.64 (t, J=7.0 Hz, 2H), 1.91-1.87 (m, 2H).

### Example 361: Preparation of Compound 207

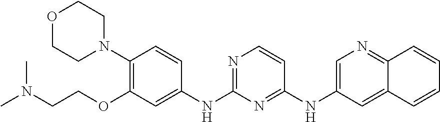 [SMILES: CN(C)CCOc1cc(Nc2nccc(Nc3cnc4ccccc4c3)n2)ccc1N1CCOCC1]

Compound 207 was prepared according to general procedure C. MS (m/z): [M+H]⁺: 486.5. ¹H NMR (500 MHz, DMSO) δ 9.85 (s, 1H), 9.10 (s, 1H), 9.00 (s, 1H), 8.92 (s, 1H), 8.09 (d, J=5.4 Hz, 1H), 7.93 (d, J=8.3 Hz, 1H), 7.71 (s, 1H), 7.62-7.57 (m, 1H), 7.53 (t, J=7.3 Hz, 1H), 7.30 (s, 1H), 7.21 (d, J=7.8 Hz, 1H), 6.82 (d, J=8.5 Hz, 1H), 6.31 (d, J=5.6 Hz, 1H), 3.87 (s, 2H), 3.78-3.71 (m, 4H), 3.00 (d, J=3.9 Hz, 4H), 2.48 (s, 2H), 2.11 (s, 6H).

### Example 362: Preparation of Compound 208

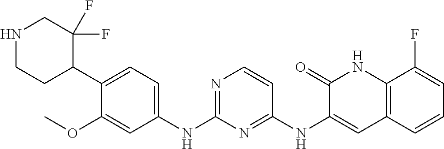 [SMILES: COc1cc(Nc2nccc(Nc3cc4cccc(F)c4[nH]c3=O)n2)ccc1C1CCNCC1(F)F]

The title compound was prepared according to procedure F starting from 4-(3,3-difluoropiperidin-4-yl)-3-methoxyaniline and 3-((2-chloropyrimidin-4-yl)amino)-8-fluoroquinolin-2(1H)-one. MS (m/z): [M+H]⁺: 497.3. ¹H NMR: (500 MHz, DMSO) δ (ppm): 12.29 (s, 1H), 9.32 (s, 11H), 9.13 (s, 1H), 9.07 (s, 1H), 8.12 (d, J=5.7 Hz, 1H), 7.29 (dt, J=19.4, 10.0 Hz, 5H), 7.16-7.08 (m, 1H), 6.83 (d, J=5.7 Hz, 1H), 3.73-3.56 (m, 4H), 3.11 (t, J=11.9 Hz, 1H), 3.00 (d, J=13.1 Hz, 1H), 2.84 (dd, J=31.9, 13.2 Hz, 1H), 2.63 (t, J=12.3 Hz, 1H), 1.99-1.88 (m, 1H), 1.68 (d, J=12.4 Hz, 1H). 1H overlap with solvent peak.

The following compounds were prepared similarly to the examples above substituting the appropriate reagents and intermediates. Some examples may require additional functional group transformations or deprotection to arrive at the final compounds.

TABLE A

| Cmpd | MS (m/z) | |
|---|---|---|
| # | [M + H]+ | ¹H NMR |
| 209 | 515.6 | 1H NMR (400 MHz, DMSO) δ 9.29 (s, 1H), |
| | | 8.88 (s, 1H), 8.32 (d, J = 1.9 Hz, |
| | | 1H), 8.07 (d, J = 2.4 Hz, 1H), 7.94 |
| | | (d, J = 5.7 Hz, 1H), 7.15 (dd, J = 8.6, 2.2 |
| | | Hz, 1H), 6.77 (d, J = 8.7 Hz, 1H), 6.12 |
| | | (d, J = 5.7 Hz, 1H), 3.87 (t, J = 6.5 Hz, |
| | | 2H), 3.60 (s, 3H), 2.97 (s, 1H), 2.93 |
| | | (s, 1H), 2.35-2.20 (m, 6H), 1.80-1.68 |
| | | (m, 6H), 1.47-1.42 (m, 4H), 1.36-1.26 (m, 6H) |
| 210 | 509.5, | 1HNMR(400 MHz, dmso) δ 9.90 (s, 1H), |
| | 511.6 | 9.15 (d, J = 2.5 Hz, 1H), 9.10 (s, |
| | | 1H), 8.98 (s, 1H), 8.19 (d, J = 3.6 Hz, 1H), |
| | | 7.96 (d, J = 8.9 Hz, 1H), 7.88 (s, |
| | | 1H), 7.64-7.61 (m, 1H), 7.20 (d, J = 8.3 Hz, |
| | | 2H), 6.71 (d, J = 8.4 Hz, 1H), |
| | | 4.34-4.30 (m, 1H), 3.62 (s, 3H), 2.57-2.53 |
| | | (m, 2H), 2.35-2.27 (m, 2H), |
| | | 2.04 (s, 6H), 1.84-1.79 (m, 2H). |
| 211 | 501.4 | 1H NMR(500 MHz, DMSO) δ 12.04 |
| | | (s, 1H), 9.04 (s, 1H), 8.76 (s, 1H), 8.62 |
| | | (d, J = 9.4 Hz, 1H), 8.34 (s, 1H), 8.03 |
| | | (d, J = 5.7 Hz, 1H), 7.28-7.20 (m, 2H), |
| | | 7.10-7.06 (m, 2H), 6.76 (d, J = 5.7 Hz, |
| | | 1H), 3.85 (s, 3H), 3.78 (s, 2H), 2.97- |
| | | 2.93 (m, 2H), 2.75-2.69 (m, 1H), 2.36-2.30 |
| | | (m, 2H), 1.97-1.84 (m, 4H). |
| 212 | 501.4 | 1HNMR(500 MHz, DMSO) δ 9.09 (s, 1H), |
| | | 8.79 (s, 1H), 8.40 (d, J = 2.8 Hz, |
| | | 2H), 8.05 (d, J = 5.7 Hz, 1H), 8.00 (d, |
| | | J = 7.1 Hz, 1H), 7.27-7.21 (m, 2H), |
| | | 7.15-7.11 (m, 1H), 6.89 (d, J = 7.8 Hz, |
| | | 1H), 6.78 (d, J = 5.7 Hz, 1H), 3.93- |
| | | 3.89 (m, 3H), 3.77 (s, 2H), 2.93 (d, |
| | | J = 11.1 Hz, 2H), 2.65-2.61 (m, 1H), 2.36- |
| | | 2.29 (m, 2H), 1.95 (d, J = 10.4 Hz, 2H), |
| | | 1.86-1.79 (m, 2H). |
| 213 | 500.5 | 1HNMR(500 MHz, DMSO) δ 9.05 (d, |
| | | J = 14.3 Hz, 1H), 8.69 (s, 1H), 8.09 (s, |
| | | 1H), 8.02 (t, J = 6.2 Hz, 1H), 7.71 (d, J = 7.7 |
| | | Hz, 1H), 7.08 (s, 2H), 7.00 (d, J = |
| | | 1.6 Hz, 2H), 6.87-6.84 (m, 1H), 6.68 |
| | | (s, 1H), 3.91-3.80 (m, 3H), 3.78 (s, |
| | | 2H), 2.94 (d, J = 10.9 Hz, 2H), 2.61-2.52 |
| | | (m, 2H), 2.37-2.28 (m, 2H), 1.88 |
| | | (d, J = 12.3 Hz, 2H), 1.82-1.71 (m, 2H). |
| 214 | 463.4 | 1HNMR(500 MHz, DMSO) δ 12.20 (s, 1H), |
| | | 9.06 (s, 1H), 8.76 (s, 1H), 8.63 (s, |
| | | 1H), 8.37 (s, 1H), 8.03 (d, J = 5.7 Hz, |
| | | 1H), 7.32-7.19 (m, 2H), 7.13-7.05 |
| | | (m, 2H), 6.76 (d, J = 5.7 Hz, 1H), |
| | | 4.03-3.97 (m, 2H), 3.85 (s, 3H), 3.52- |
| | | 3.45 (m, 2H), 3.02-2.94 (m, 1H), |
| | | 2.10-1.77 (m, 4H). |
| 215 | 490.4 | 1H NMR (500 MHz, DMSO) δ 9.01 |
| | | (s, 1H), 8.72 (s, 1H), 8.09 (s, 1H), 8.03 (d, |
| | | J = 5.7 Hz, 1H), 7.65 (d, J = 7.7 Hz, 1H), |
| | | 7.21-7.13 (m, 1H), 7.13-7.01 (m, |
| | | 2H), 6.96 (s, 1H), 6.86 (d, J = 8.0 Hz, 1H), |
| | | 6.70 (d, J = 5.7 Hz, 1H), 4.11 (s, |
| | | 1H), 3.80 (s, 3H), 2.48-2.43 (m, 1H), |
| | | 2.00-1.84 (m, 2H), 1.76-1.58 (m, |
| | | 4H), 1.54-1.42 (m, 2H), 1.17 (s, 3H). |
| 216 | 490.4 | 1H NMR (500 MHz, DMSO) δ 9.02 (s, |
| | | 1H), 8.73 (s, 1H), 8.07 (s, 1H). 8.02 (d, |
| | | J = 5.7 Hz, 1H), 7.68 (d, J = 7.9 Hz, |
| | | 1H), 7.23-7.15 (m, 1H), 7.10 (s, 1H), |
| | | 7.05-6.94 (m, 2H), 6.87 (d, J = 8.2 Hz, 1H), |
| | | 6.70 (d, J = 5.7 Hz, 1H), 4.36 (s, |
| | | 1H), 3.81 (s, 3H), 2.58-2.53 (m, 1H), |
| | | 1.86-1.79 (m, 2H), 1.72-1.66 (m, |
| | | 2H), 1,65-1.52 (m, 4H), 1.24 (s, 3H). |
| 217 | 527.4 | 1HNMR(500 MHz, DMSO) δ 12.16 (s, |
| | | 1H), 8.94 (s, 1H), 8.49 (s, 2H), 8.16 (s, |
| | | 1H), 8.08 (s, 1H), 7.97 (d, J = 5.6 Hz, |
| | | 1H), 7.25-7.19 (m, 1H), 7.09 (s, 1H), |
| | | 6.68 (d, J = 13.2 Hz, 1H), 6.51 (s, 1H), |
| | | 6.36-6.11 (m, 1H), 3.95-3.76 (m, |
| | | 2H), 3.61-3.44 (m, 3H), 3.32-3.23 |
| | | (m, 2H), 2.87-2.50 (m, 6H). |
| 218 | 513.4 | 1HNMR(500 MHz, DMSO) δ 8.94 (s, 1H), |
| | | 8.55 (s, 1H), 8.10 (d, J = 10.1 Hz, |
| | | 2H), 7.95 (d, J = 5.5 Hz, 1H), 7.10 (s, |
| | | 1H), 6.99-6.93 (m, 1H), 6.58 (s, 1H), |
| | | 6.52 (s, 1H), 6.17-5.90 (m, 2H), 4.37 (d, |
| | | J = 12.3 Hz, 1H), 4.04 (d, J = 12.4 |
| | | Hz, 1H), 3.78 (s, 3H), 3.10-3.03 (m, 2H), |
| | | 2.91-2.85 (m, 1H), 2.82-2.74 |
| | | (m, 2H), 2.69 (s, 1H), 2.54-2.51 (m, 1H). |
| 219 | 462.5 | 1HNMR(500 MHz, DMSO) δ 12.30 (s, |
| | | 1H), 9.24 (s, 1H), 9.11 (s, 1H), 9.05 (s, |
| | | 1H), 8.11 (d, J = 5.7 Hz, 1H), 7.32-7.20 |
| | | (m, 4H), 7.12 (d, J = 8.8 Hz, 2H), |
| | | 6.80 (d, J = 5.7 Hz, 1H), 3.96 (d, J = 10.2 |
| | | Hz, 2H), 3.71 (s, 3H), 3.49-3.43 |
| | | (m, 2H), 3.13-3.07 (m, 1H), 1.72-1.64 (m, 4H). |
| 220 | 462.5 | 1HNMR(500 MHz, DMSO) δ 12.26 |
| | | (s, 1H), 9.02 (s, 1H), 8.79 (s, 1H), 8.14 (s, |
| | | 1H), 8.04 (d, J = 5.7 Hz, 1H), 7.68 (d, |
| | | J = 7.8 Hz, 1H), 7.25-7.21 (m, 1H), |
| | | 7.15-7.08 (m, 2H), 7.00 (d, J = 1.5 Hz, 1H), |
| | | 6.87-6.84 (m, 1H), 6.74 (d, J = |
| | | 5.7 Hz, 1H), 4.02-3.97 (m, 2H), 3.81 |
| | | (s, 3H), 3.51-3.45 (m, 2H), 1.80- |
| | | 1.73 (m, 4H). |
| 221 | 489.5 | 1H NMR (500 MHz, DMSO) δ 8.93 (s, 1H), |
| | | 8.33-8.19 (m, 3H), 8.13 (s, 1H), |
| | | 8.04 (s, 1H), 7.98 (d, J = 5.3 Hz, 1H), |
| | | 7.27-7.19 (m, 1H), 7.19-7.10 (m, |
| | | 1H), 6.66 (d, J = 5.7 Hz, 1H), 6.43 (s, 1H), |
| | | 3.76 (s, 3H), 3.56-3.46 (m, 2H), |
| | | 3.44-3.35 (m, 2H), 2.98-2.87 (m, 2H), |
| | | 0.57-0.48 (m, 4H). |
| 222 | 497.3 | 1H NMR (500 MHz, DMSO): δ 12.11 (s, 1H), |
| | | 9.06 (s, 1H), 8.85 (s, 1H), 8.14 |
| | | (s, 1H), 8.06 (d, J = 5.7 Hz, 1H), 7.81 (d, |
| | | J = 8.1 Hz, 1H), 7.26-7.16 (m, 2H), |
| | | 7.10 (td, J = 7.9, 4.9 Hz, 1H), 7.00 (s, 1H), |
| | | 6.92 (d, J = 8.1 Hz, 1H), 6.78 (d, J = |
| | | 5.7 Hz, 1H), 3.82 (s, 3H), 3.23 (ddd, |
| | | J = 12.8, 8.3, 3.9 Hz, 1H), 3.14 (t, J = |
| | | 11.4 Hz, 1H), 3.03 (d, J = 12.1 Hz, 1H), 2.86 |
| | | (dd, J = 32.1, 13.3 Hz, 1H), 2.65 |
| | | (t, J = 11.5 Hz, 1H), 2.09-1.98 (m, 1H), |
| | | 1.82 (d, J = 13.2 Hz, 1H). 1H overlap |
| | | with solvent peak. |
| 223 | 462.3 | 1H NMR (500 MHz, DMSO): δ 9.36 |
| | | (s, 1H), 9.17 (s, 1H), 9.02 (s, 1H), 8.48 |
| | | (d, J = 1.6 Hz, 1H), 8.12 (d, J = 5.7 Hz, |
| | | 1H), 7.62 (s, 1H), 7.38 (dd, J = 7.6, 2.4 |
| | | Hz, 1H), 7.25 (dd, J = 10.7, 7.7 Hz, 1H), |
| | | 7.18-7.12 (m, 1H), 6.85 (d, J = 5.7 |
| | | Hz, 1H), 3.73 (s, 3H), 3.11 (dd, J = 28.0, |
| | | 11.9 Hz, 3H), 2.65 (d, J = 11.9 Hz, |
| | | 2H), 1.81-1.70 (m, 2H), 1.67 (d, J = 13.4 |
| | | Hz, 2H). 2H overlap with solvent |
| | | peak. |
| 224 | 463.4 | 1HNMR(500 MHz, DMSO) δ 12.11 (s, 1H), |
| | | 9.07 (s, 1H), 8.76 (s, 1H), 8.37 (s, |
| | | 1H), 8.06-8.00 (m, 2H), 7.25-7.18 (m, 2H), |
| | | 7.14-7.10 (m, 1H), 6.89 (d, J = |
| | | 7.8 Hz, 1H), 6.77 (d, J = 5.7 Hz, 1H), |
| | | 4.01-3.97 (m, 2H), 3.91 (s, 3H), 3.51- |
| | | 3.46 (m, 2H), 2.92-2.86 (m, 1H), |
| | | 1.87-1.78 (m, 4H). |
| 225 | 519.4 | 1H NMR (500 MHz, DMSO) δ 12.21 |
| | | (s, 1H), 8.94 (s, 1H), 8.40 (s, 1H), 8.15 |
| | | (s, 1H), 8.08 (s, 1H), 7.97 (d, J = 5.3 Hz, |
| | | 1H), 7.27-7.16 (m, 1H), 7.13-7.01 |
| | | (m, 1H), 6.67 (d, J = 5.7 Hz, 1H), 6.51 (s, |
| | | 1H), 4.60 (dd, J = 6.5 Hz, 2H), 4.52 |
| | | (dd, J = 6.1 Hz, 2H), 3.78 (s, 3H), |
| | | 3.61-3.55 (m, 4H), 3.51-3.45 (m, 1H). |
| | | 2.45-2.39 (m, 4H). |
| 226 | 502.3 | 1HNMR(500 MHz, DMSO) δ 8.95 (s, 1H), |
| | | 8.69-8.45 (m, 1H), 8.34 (s, 1H), |
| | | 8.17 (s, 1H), 8.08 (s, 1H), 7.98 (d, J = 5.5 |
| | | Hz, 1H), 7.23-7.18 (m, 1H), 7.10 |
| | | (s, 2H), 6.67 (d, J = 5.7 Hz, 1H), 6.54 |
| | | (s, 1H), 3.85 (s, 2H), 3.78 (s, 3H), 3.62 |
| | | (s, 4H), 2.68-2.59 (m, 4H). |
| 227 | 546.4 | 1H NMR (500 MHz, DMSO) δ 12.21 (s, |
| | | 1H), 8.93 (s, 1H), 8.22 (s, 1H), 7.98 |
| | | (d, J = 5.6 Hz, 1H), 7.58 (s, 1H), |
| | | 7.25-7.16 (m, 1H), 7.14-7.01 (m, 1H), |
| | | 6.66 (d, J = 5.7 Hz, 1H), 6.47 (d, J = 8.3 |
| | | Hz, 1H), 6.11 (s, 1H), 4.31-4.20 (m, |
| | | 2H), 3.79 (s, 3H), 3.17-3.08 (m, 2H), |
| | | 1.84-1.68 (m, 4H). |
| 228 | 546.4 | 1H NMR (500 MHz, DMSO) δ 12.29 (s, |
| | | 1H), 9.17 (s, 1H), 9.11 (s, 1H), 8.96 |
| | | (s, 1H), 8.18 (s, 1H), 8.09 (d, J = 5.6 Hz, |
| | | 1H), 7.49 (s, 1H), 7.32 (s, 1H), 7.28- |
| | | 7.20 (m, 1H), 7.20-7.09 (m, 1H), 6.80 (d, |
| | | J = 5.7 Hz, 1H), 5.97 (s, 1H), 3.83- |
| | | 3.68 (m, 5H), 3.10-2.99 (m, 2H), 1.91-1.80 |
| | | (m, 2H), 1.80-1.70 (m, 2H). |
| 229 | 505.3 | 1HNMR(500 MHz, DMSO) δ 9.15 (s, 1H), |
| | | 9.11 (s, 1H), 8.95 (s, 1H), 8.29 (s, |
| | | 1H), 8.13-8.06 (m, 2H), 7.48 (s, 1H), |
| | | 7.35-7.21 (m, 2H), 7.11 (s, 1H), 6.79 |
| | | (d, J = 5.7 Hz, 1H), 3.71 (s, 3H), 3.25 |
| | | (s, 2H), 3.01 (s, 2H), 2.63-2.57 (m, |
| | | 2H), 2.19 (s, 3H), 1.06 (s, 6H). |
| 230 | 492.4 | 1H NMR (500 MHz, DMSO) δ 8.93 |
| | | (s, 1H), 8.12 (s, 1H), 8.04 (s, 1H). 7.98 (d, |
| | | J = 5.4 Hz, 1H), 7.26-7.16 (m, 1H), |
| | | 7.15-7.05 (m, 1H), 6.66 (d, J = 5.7 Hz, |
| | | 1H), 6.48 (s, 1H), 3.94-3.85 (m, 2H), 3.77 |
| | | (s, 3H), 3.45-3.38 (m, 2H), 1.64- |
| | | 1.50 (m, 4H), 1.20 (s, 3H). |
| 231 | 492.4 | 1H NMR (500 MHz, DMSO) δ 8.92 (s, |
| | | 1H), 8.31 (s, 1H), 8.19 (s, 1H), 7.98 (d, |
| | | J = 5.6 Hz, 1H), 7.56-7.46 (m, 1H), |
| | | 7.26-7.18 (m, 1H), 7.15-7.09 (m, 1H), |
| | | 6.65 (d, J = 5.7 Hz, 1H), 6.40 (d, J = 8.3 |
| | | Hz, 1H), 3.88-3.80 (m, 2H), 3.77 (8, |
| | | 3H), 3.42-3.33 (m, 2H), 1.64-1.52 |
| | | (m, 4H), 1.20 (s, 3H). |
| 232 | 545.5 | 1H NMR (500 MHz, DMSO) δ 12.28 |
| | | (s, 1H), 9.18 (s, 1H), 9.11 (s, 1H), 8.95 |
| | | (s, 1H), 8.15 (s, 1H), 8.08 (d, J = 3.5 Hz, |
| | | 1H), 7.52 (s, 1H), 7.34-7.19 (m, |
| | | 2H), 7.15-7.06 (m, 1H). 6.79 (d, J = 5.6 |
| | | Hz, 1H), 3.72 (s, 3H), 3.29 (s, 2H), |
| | | 3.26-3.19 (m, 4H), 2.81-2.75 (m, 4H). |
| 233 | 513.5 | 1HNMR(500 MHz, DMSO) δ 12.27 (s, |
| | | 1H), 9.18 (s, 1H), 9.11 (s, 1H), 8.95 (s, |
| | | 1H), 8.17 (d, J = 2.0 Hz, 1H), 8.08 (d, |
| | | J = 5.7 Hz, 1H), 7.52 (s, 1H), 7.33- |
| | | 7.21 (m, 2H), 7.12 (s, 1H), 6.79 (d, J = 5.7 |
| | | Hz, 1H), 3.71 (s, 3H), 3.68-3.61 |
| | | (m, 2H), 3.12 (s, 2H), 3.00-2.96 (m, 1H), |
| | | 2.88-2.73 (m, 4H). |
| 234 | 476.5 | 1H NMR (400 MHz, DMSO): δ 12.19 (s, |
| | | 1H), 9.06 (s, 1H), 9.02 (s, 1H), 8.89 |
| | | (s, 1H), 8.05 (d, J = 5.7 Hz, 1H), 8.00 (d, |
| | | J = 1.9 Hz, 1H), 7.41-7.08 (m, 4H), |
| | | 6.73 (d, J = 5.8 Hz, 1H), 4.73 (s, 4H), |
| | | 4.15 (s, 4H), 3.66 (s, 3H). |
| 235 | 463.4 | 1H NMR (500 MHz, DMSO): δ 9.13 (s, 1H), |
| | | 9.11 (s, 1H), 8.96 (s, 1H), 8.14 |
| | | (d, J = 2.1 Hz, 1H), 8.08 (d, J = 5.7 Hz, |
| | | 1H), 7.48 (s, 1H), 7.35-7.05 (m, 2H), |
| | | 6.81-6.75 (m, 1H), 3.72 (s, 4H), 3.20 |
| | | (s, 4H), 2.88-2.83 (m, 4H). 2H |
| | | overlap with solvent peak |
| 236 | 502.1 | 1H NMR (500 MHz, DMSO): δ 12.28 (s, |
| | | 1H), 9.18 (s, 1H), 9.11 (s, 1H), 8.97 |
| | | (s, 1H), 8.16 (d, J = 1.9 Hz, 1H), 8.09 (d, |
| | | J = 5.7 Hz, 1H), 7.54 (s, 1H), 7.34- |
| | | 7.07 (m, 3H), 6.80 (d, J = 5.7 Hz, 1H), |
| | | 3.80 (s, 2H), 3.73 (s, 3H), 2.67-2.64 |
| | | (m, 4H). 4H overlap with water peak. |
| 237 | 519.5 | 1H NMR (400 MHz, DMSO): δ 12.28 |
| | | (s, 1H), 9.17 (s, 1H), 9.12 (s, 1H), 8.97 |
| | | (s, 1H), 8.14 (d, J = 1.4 Hz, 1H), 8.08 (d, |
| | | J = 5.7 Hz, 1H), 7.51 (s, 1H), 7.35- |
| | | 7.09 (m, 3H), 6.79 (d, J = 5.7 Hz, 1H), 4.57 (t, |
| | | J = 6.5 Hz, 2H), 4.50 (t, J = 6.1 |
| | | Hz, 2H), 3.71 (s, 3H), 3.51-3.43 (m, 1H), |
| | | 2.45-2.40 (m, 4H). 4H overlap |
| | | with water peak. |
| 238 | 520.8 | 1H NMR (500 MHz, DMSO): δ 12.28 |
| | | (s, 1H), 9.15 (s, 1H), 9.11 (s, 1H), 8.95 |
| | | (s, 1H), 8.21 (s, 1H), 8.15 (d, J = 1.8 Hz, 1H), |
| | | 8.08 (d, J = 5.7 Hz, 1H), 7.48 (s, |
| | | 1H), 7.33-7.10 (m, 3H), 6.79 (d, J = 5.7 Hz, |
| | | 1H), 4.45 (s, 1H), 3.81 (d, J = |
| | | 11.2 Hz, 1H), 3.71 (d, J = 11.0 Hz, 4H), 3.05 |
| | | (d, J = 11.3 Hz, 1H), 2.88 (t, J = |
| | | 10.3 Hz, 1H), 2.65 (dd, J = 11.9, 6.4 Hz, 2H), |
| | | 2.46 (d, J = 10.7 Hz, 1H), 1.14 |
| | | (s, 3H), 1.13 (s, 3H). |
| 239 | 531.4 | 1HNMR(500 MHz, DMSO) δ 12.33 |
| | | (s, 1H), 9.25 (s, 1H), 9.16 (s, 1H), 8.97 (s, |
| | | 1H), 8.51 (s, 1H), 8.19 (d, J = 1.8 Hz, 1H), |
| | | 8.09 (d, J = 5.7 Hz, 1H), 7.54 (s, |
| | | 1H), 7.27-7.23 (m, 1H), 7.11 (s, 1H), 6.81 |
| | | (d, J = 5.7 Hz, 1H), 3.80-3.61 |
| | | (m, 5H), 3.55 (s, 1H), 3.02-2.89 (m, 2H), |
| | | 2.88-2.82 (m, 2H), 2.79-2.72 |
| | | (m, 1H). |
| 240 | 511.5 | 1H NMR (500 MHz, DMSO) δ 9.22 (s, |
| | | 1H), 9.13 (s, 1H), 8.86 (br, 1H), 8.24 |
| | | (d, J = 2.0 Hz, 1H), 8.08 (d, J = 5.7 Hz, |
| | | 1H), 7.61 (s, 1H), 7.23 (s, 1H), 7.19- |
| | | 7.04 (m, 2H), 6.75 (d, J = 5.5 Hz, 1H), |
| | | 3.93-3.85 (m, 2H), 3.78-3.71 (m, |
| | | 4H), 3.70-3.61 (m, 2H). 3.19-3.06 (m, 4H). |
| 241 | 464.3 | 1HNMR(500 MHz, DMSO) δ 12.27 (s, 1H), |
| | | 9.18 (s, 1H), 9.12 (s, 1H), 8.94 (s, |
| | | 1H), 8.16 (d, J = 1.8 Hz, 1H), 8.08 (d, |
| | | J = 5.7 Hz, 1H), 7.54 (s, 1H), 7.34- |
| | | 7.21 (m, 2H), 7.10 (d, J = 4.7 Hz, 1H), |
| | | 6.78 (d, J = 5.7 Hz, 1H), 3.77-3.74 (m, |
| | | 4H), 3.72 (s, 3H), 3.28-3.25 (m, 4H). |
| 242 | 509.5 | 1HNMR(500 MHz, DMSO) δ 9.23 (s, |
| | | 1H), 8.84 (s, 1H), 8.36 (s, 1H), 8.33 (s, |
| | | 1H), 8.23 (d, J = 3.0 Hz, 1H), 7.34-7.30 |
| | | (m, 1H), 7.26-7.14 (m, 3H), 6.81 |
| | | (d, J = 8.6 Hz, 1H), 6.27 (s, 1H), 4.39-4.33 |
| | | (m, 1H), 3.69 (s, 3H), 2.61-2.56 |
| | | (m, 2H), 2.34-2.29 (m, 1H), 2.13 (s, 6H), |
| | | 1.87-1.81 (m, 2H). |
| 243 | 447.5 | 1H NMR (500 MHz, DMSO): δ 12.29 (s, |
| | | 1H), 9.22 (s, 1H), 9.08 (s, 1H), 9.04 |
| | | (s, 1H), 8.10 (d, J = 5.8 Hz, 1H), 7.37 (s, |
| | | 1H), 7.31-7.17 (m, 3H), 7.11 (d, J = |
| | | 8.1 Hz, 1H), 7.07-7.03 (m, 1H), 6.80 (d, |
| | | J = 5.7 Hz, 1H), 4.96-4.89 (m, |
| | | 1H), 3.27-3.20 (m, 1H), 2.91 (dd, J = 15.3, |
| | | 7.7 Hz, 1H), 2.60-2.55 (m, 1H), |
| | | 2.49-2.45 (m, 1H), 2.22 (s, 6H). |
| 244 | 492.5 | 1HNMR(500 MHz, DMSO) δ 12.28 (s, |
| | | 1H), 9.12 (s, 1H), 9.00 (s, 1H), 8.91 (s, |
| | | 1H), 8.09-8.01 (m, 2H), 7.55 (s, 2H), 7.10-7.00 |
| | | (m, 2H), 6.74 (d, J = 5.8 |
| | | Hz, 1H), 4.90-4.83 (m, 1H), 3.71 (s, 3H), |
| | | 2.63-2.56 (m, 2H), 2.39-2.33 |
| | | (m, 1H), 2.06 (s, 6H), 1.88-1.80 (m, 2H). |
| 245 | 479.4 | 1HNMR(500 MHz, DMSO) δ 12.24 (s, 1H), |
| | | 9.06 (d, J = 8.4 Hz, 2H), 8.93 (s, |
| | | 1H), 8.07 (d, J = 5.7 Hz, 1H), 7.56 (s, 1H), |
| | | 7.42-7.34 (m, 2H), 7.28 (d, J = 8.7 |
| | | Hz, 1H), 7.01 (s, 1H), 6.82 (d, J = 8.7 Hz, |
| | | 1H), 6.74 (d, J = 5.7 Hz, 1H), 4.34- |
| | | 4.27 (m, 2H), 3.97-3.92 (m, 1H), 2.53-2.51 |
| | | (m, 2H), 2.23 (s, 6H). |
| 246 | 492.3 | 1HNMR(500 MHz, DMSO) δ 12.29 (s, 1H), |
| | | 9.13 (d, J = 21.2 Hz, 2H), 8.97- |
| | | 8.89 (m, 1H), 8.09 (d, J = 5.7 Hz, 1H), 8.03 |
| | | (d, J = 1.9 Hz, 1H), 7.53 (s, 1H), |
| | | 7.30-7.21 (m, 2H), 7.15 (s, 1H), 6.80 (d, |
| | | J = 5.7 Hz, 1H), 4.91-4.86 (m, |
| | | 1H), 3.71 (s, 3H), 2.66-2.57 (m, 2H), 2.37 (d, |
| | | J = 5.2 Hz, 1H), 2.06 (s, 7H), |
| | | 1.88-1.80 (m, 2H). |
| 247 | 533, | 1H NMR (500 MHz, DMSO): δ 12.35 (s, |
| | 535 | 1H), 9.08 (d, J = 4.5 Hz, 2H), 9.00 |
| | | (s, 1H), 8.08 (d, J = 5.7 Hz, 1H), 7.56 (s, 1H), |
| | | 7.39 (dd, J = 8.7, 2.3 Hz, 1H), |
| | | 7.33 (s, 1H), 7.29 (d, J = 8.7 Hz, 1H), 7.25 (d, |
| | | J = 7.4 Hz, 1H), 6.93 (d, J = 8.7 |
| | | Hz, 1H), 6.76 (d, J = 5.7 Hz, 1H), |
| | | 4.38-4.30 (m, 1H), 3.73 (s, 3H), 2.23- |
| | | 2.14 (m, 7H), 1.97-1.89 (m, 2H), 1.76-1.63 |
| | | (m, 2H), 1.56-1.47 (m, 4H). |
| 248 | 443.5 | 1HNMR (500 MHz, DMSO) δ 9.06 (s, |
| | | 1H), 8.93 (s, 1H), 8.88-8.85 (m, 1H), |
| | | 8.53 (s, 1H), 8.04 (d, J = 5.7 Hz, 1H), 7.54 |
| | | (d, J = 8.5 Hz, 2H), 7.38-7.35 (m, |
| | | 1H), 7.28 (s, 1H), 7.19-7.16 (m, 1H), |
| | | 6.81-6.78 (m, 2H), 6.70 (d, J = 5.7 |
| | | Hz, 1H), 4.72-4.69 (m, 1H), 2.85-2.82 |
| | | (m, 1H), 2.35-2.31 (m, 2H), 2.22- |
| | | 2.06 (m, 8H), 2.01 (s, 1H). |
| 249 | 473.4 | 1H NMR (500 MHz, DMSO) δ 12.23 (s, |
| | | 1H), 9.08 (s, 1H), 8.96 (s, 2H), 8.06 |
| | | (d, J = 5.7 Hz, 1H), 7.43 (s, 1H), 7.39-7.35 |
| | | (m, 1H), 7.30 (d, J = 8.1 Hz, 1H), |
| | | 7.27 (s, 1H), 7.23 (d, J = 8.4 Hz, 1H), 7.18 |
| | | (t, J = 7.2 Hz, 1H), 6.72 (dd, J = |
| | | 7.2, 4.2 Hz, 2H), 4.78-4.63 (m, 1H), |
| | | 3.70 (s, 3H), 2.90 (s, 1H), 2.43-2.27 |
| | | (m, 2H), 2.27-2.16 (m, 2H), 2.12 (s, 6H). |
| 250 | 501.3 | 1H NMR (500 MHz, DMSO) δ 12.23 |
| | | (s, 1H), 9.09 (s, 1H), 8.97 (s, 2H), 8.07 |
| | | (d, J = 5.7 Hz, 1H), 7.45 (s, 1H), 7.39-7.33 |
| | | (m, 1H), 7.30 (d, J = 7.6 Hz, 2H), |
| | | 7.23 (d, J = 8.2 Hz, 1H), 7.18 (t, J = 7.4 Hz, |
| | | 1H), 6.93 (d, J = 8.7 Hz, 1H), 6.73 |
| | | (d, J = 5.8 Hz, 1H), 4.00 (t, J = 6.4 Hz, |
| | | 2H), 3.70 (s, 3H), 2.45 (s, 4H), 1.93 (s, |
| | | 2H), 1.55 (s, 4H), 1.42 (s, 2H). |
| | | *one peak missing due to overlap. |
| 251 | 473.4 | 1H NMR (500 MHz, DMSO) δ 12.22 (s, |
| | | 1H), 9.06 (s, 1H), 8.94 (s, 1H), 8.90 |
| | | (s, 1H), 8.05 (d, J = 5.7 Hz, 1H), 7.55 (d, |
| | | J = 7.8 Hz, 2H), 7.45 (s, 1H), 7.36 |
| | | (dd, J = 11.1, 4.1 Hz, 1H), 7.29 (d, J = 8.1 |
| | | Hz, 1H), 7.19 (t, J = 7.3 Hz, 1H), |
| | | 6.92 (d, J = 9.0 Hz, 2H), 6.71 (d, J = 5.7 Hz, |
| | | 1H), 4.02 (t, J = 6.4 Hz, 2H), 3.62- |
| | | 3.56 (m, 4H), 2.45 (t, J = 7.2 Hz, 2H), 2.37 |
| | | (dd, J = 5.9, 4.0 Hz, 4H), 1.95- |
| | | 1.87 (m, 2H). |
| 252 | 509.4 | 1H NMR (500 MHz, DMSO) δ 9.36 (s, |
| | | 1H), 8,92-8.84 (m, 2H), 8.62 (s, 1H), |
| | | 8.11 (s, 1H), 7.93-7.83 (m, 1H), 7.75-7.65 |
| | | (m, 1H), 7.59 (s, 1H), 6.27 (s, |
| | | 2H), 5.76 (s, 1H), 5.45 (s, 1H), 4.90-4.82 |
| | | (m, 1H), 3.70 (s, 3H), 2.61-2.53 |
| | | (m, 2H), 2.39-2.31 (m, 1H), 2.06 |
| | | (s, 6H), 1.87-1.79 (m, 2H). |
| 253 | 518.2 | 1H NMR (500 MHz, DMSO) δ 10.02 |
| | (M-H) | (s, 1H), 9.19 (s, 1H), 8.96 (s, 2H), 8.11 |
| | | (d, J = 5.7 Hz, 1H), 8.05 (s, 1H), 7.93 (t, |
| | | J = 9.8 Hz, 1H), 7.75 (s, 1H), 7.56 (s, |
| | | 1H), 6.35 (d, J = 5.7 Hz, 1H), 4.69 (t, |
| | | J = 7.8 Hz, 1H), 3.68 (s, 3H), 2.55 (s, |
| | | 1H), 2.30 (s, 6H), 1.96 (s, 1H), |
| | | 1.28 (s, 3H), 1.05 (s, 3H). |
| 254 | 522.7 | 1H NMR (500 MHz, DMSO) δ 9.16 (s, |
| | | 1H), 9.11 (s, 1H), 8.93 (s, 1H), 8.09 (d, |
| | | J = 5.7 Hz, 1H), 7.99 (s, 1H), 7.54 (s, 1H), |
| | | 7.27 (d, J = 8.6 Hz, 1H), 7.24 (d, J = |
| | | 8.4 Hz, 1H), 7.16-7.07 (m, 1H), 6.80 (d, J = 5.7 |
| | | Hz, 1H), 4.68 (t, J = 7.8 Hz, |
| | | 1H), 3.70 (8, 3H), 2.45 (dt, J = 10.5, 7.0 Hz, |
| | | 1H), 2.02 (8, 6H), 1.91 (dd, J = |
| | | 9.4, 7.0 Hz, 1H), 1.83-1.73 (m, 1H), |
| | | 1.26 (s, 3H), 0.98 (s, 3H). |
| 255 | 519.5 | 1HNMR(500 MHz, DMSO) δ 12.50 (s, |
| | | 1H), 9.70 (s, 1H), 9.38 (s, 1H), 8.83 (s, |
| | | 1H), 8.26 (s, 1H), 8.23 (d, J = 3.1 Hz, 1H), 8.07 |
| | | (d, J = 2.1 Hz, 1H), 8.03 (d, J = |
| | | 8.5 Hz, 1H). 7.84 (s, 1H), 7.74-7.70 (m, |
| | | 1H), 7.68-7.63 (m, 1H), 7.52 (s, |
| | | 1H), 4.93-4.87 (m, 1H), 3.70 (s, 4H), |
| | | 2.66-2.58 (m, 3H), 2.42-2.36 |
| | | (m, 1H), 2.08-2.02 (m, 8H), 1.90-1.83 (m, 2H). |
| 256 | 504.6 | 1H NMR (500 MHz, DMSO) δ 11.64 |
| | | (s, 1H), 9.08 (s, 1H), 8.80 (s, 1H), 8.26 |
| | | (s, 1H), 8.07 (d, J = 5.6 Hz, 1H), 7.84 (s, |
| | | 1H), 7.21-7.09 (m, 2H), 6.78 (d, J = |
| | | 8.7 Hz, 1H), 6.21 (d, J = 5.7 Hz, 1H), 4.33 |
| | | (dt, J = 14.3, 7.3 Hz, 1H), 3.66 (s, |
| | | 3H), 2.79 (t, J = 6.3 Hz, 2H), 2.68 (s, 2H), |
| | | 2.61-2.55 (m, 2H), 2.31 (dt, J = |
| | | 15.7, 7.9 Hz, 1H), 2.06 (s, 6H), 1.88-1.79 (m, |
| | | 4H), 1.75 (d, J = 5.7 Hz, 2H). |
| 257 | 514.2 | 1H NMR (500 MHz, DMSO) δ 10.00 |
| | | (s, 1H), 9.23 (s, 1H), 9.08-8.87 (m, |
| | | 2H), 8.13 (d, J = 5.7 Hz, 1H), 8.11 (d, |
| | | J = 2.1 Hz, 1H), 7.95 (d, J = 8.9 Hz, |
| | | 1Hf), 7.91 (s, 1H), 7.68 (s, 1H), 7.60 (dd, J = 8.9, |
| | | 2.4 Hz, 1H), 6.37 (d, J = 5.7 |
| | | Hz, 1H), 5.57-5.42 (m, 1H), 3.73 (s, 3H), |
| | | 3.14 (td, J = 13.7, 10.5 Hz, 1H), |
| | | 2.98-2.85 (m, 2H), 2.71-2.63 (m, 1H), |
| | | 2.07-2.01 (m, 1H), 1,79-1.70 (m, |
| | | 1H). |
| 258 | 520.5 | 1H NMR (500 MHz, DMSO) δ 10.08 (s, |
| | | 1H), 9.17 (s, 1H), 8.99 (s, 1H), 8.94 |
| | | (s, 1H), 8.36 (s, 1H), 8.12 (d, J = 5.6 Hz, 1H), |
| | | 8.08 (s, 1H), 7.93 (d, J = 8.8 Hz, |
| | | 1H), 7.86 (s, 1H), 7.58 (dd, J = 8.8, 1.9 Hz, |
| | | 1H), 7.54 (s, 1H), 6.36 (d, J = 5.6 |
| | | Hz, 1H), 4.74-4.67 (m, 1H), 3.70 (s, 3H), |
| | | 2.47-2.40 (m, 1H), 2.02 (s, 6H), |
| | | 1.95-1.87 (m, 1H), 1.79 (dd, J = 19.1, |
| | | 9.6 Hz, 1H), 1.24 (s, 3H), 0.95 (s, 3H). |
| 259 | 522.5 | 1H NMR (500 MHz, DMSO) δ 9.32 |
| | | (s, 1H), 8.97 (s, 1H), 8.24 (s, 1H, |
| | | HCOOH), 7.98 (d, J = 4.9 Hz, 2H), 7.83 (d, |
| | | J = 2.0 Hz, 1H), 7.29 (s, 1H), 7.22 |
| | | (d, J = 8.7 Hz, 1H), 6.71 (d, J = 8.7 Hz, |
| | | 1H), 6.13 (d, J = 5.7 Hz, 1H), 4.46 (dt, |
| | | J = 8.5, 4.2 Hz, 1H), 4.30 (td, J = 11.5, |
| | | 4.8 Hz, 2H), 3.99 (dd, J = 11.5, 7.3 Hz, |
| | | 1H), 3.67 (s, 3H), 2.57 (ddd, J = 9.8, 6.2, |
| | | 3.7 Hz, 4H), 2.39 (dt, J = 15.0, 7.6 |
| | | Hz, 1H), 2.25 (s, 6H), 2.10 (s, 6H), 1.85 |
| | | (ddd, J = 16.2, 8.4, 2.8 Hz, 2H). |
| 260 | 435.5 | 1HNMR(500 MHz, DMSO) δ 9.26 |
| | | (s, 1H), 8.97 (s, 1H), 7.98-7.92 (m, 2H), |
| | | 7.79 (d, J = 2.3 Hz, 1H), 7.54 (d, J = 9.0 Hz, |
| | | 2H), 6.76-6.72 (m, 2H), 6.10 (d, |
| | | J = 5.7 Hz, 1H), 4.38-4.32 (m, 3H), |
| | | 4.26-4.24 (m, 2H), 2.63-2.56 (m, 2H), |
| | | 2.35-2.30 (m, 1H), 2.04 (s, 6H), |
| | | 1.82-1.75 (m, 2H). |
| 261 | 478.0 | 1H NMR (500 MHz, DMSO) δ 9.19 (s, 1H), |
| | | 9.12 (s, 1H), 8.95 (s, 1H), 8.32 (s, |
| | | 1H), 8.09 (d, J = 5.7 Hz, 1H), 8.03 (s, 1H), |
| | | 7.57 (s, 1H), 7.27 (d, J = 8.9 Hz, |
| | | 1H), 7.25 (d, J = 9.0 Hz, 1H), 7.18-7.11 (m, |
| | | 1H), 6.81 (d, J = 5.7 Hz, 1H), |
| | | 5.16 (dd, J = 10.8, 6.8 Hz, 1H), 3.72 (s, 3H), |
| | | 3.15-3.10 (m, 2H), 2.85 (t, J = |
| | | 9.8 Hz, 2H), 2.11-2.03 (m, 2H), |
| | | 1.79-1.62 (m, 2H). |
| 262 | 457.4 | 1H NMR (400 MHz, DMSO) δ 10.13 (s, |
| | | 1H), 8.69 (s, 1H), 8.58 (s, 1H), 7.98 |
| | | (d, J = 5.7 Hz, 1H), 7.89 (d, J = 8.2 Hz, 1H), |
| | | 7.83 (d, J = 1.3 Hz, 1H), 7.78 (d, J = |
| | | 8.3 Hz, 1H), 7.72-7.65 (m, 1H), 7.57-7.48 (m, |
| | | 1H), 7.23 (d, J = 2.4 Hz, |
| | | 1H), 7.14 (dd, J = 8.6, 2.3 Hz, 1H), 7.00 (dd, |
| | | J = 5.7, 1.7 Hz, 1H), 6.77 (d, J = |
| | | 8.6 Hz, 1H), 4.31 (p, J = 7.1 Hz, 1H), |
| | | 3.72 (s, 3H), 2.60-2.51 (m, 2H), 2.32 |
| | | (dd, J = 15.2, 8.3 Hz, 1H), 2.04 (s, 6H), |
| | | 1.81 (dd, J = 19.2, 8.4 Hz, 2H). |
| 263 | 516.3 | 1H NMR (500 MHz, DMSO) δ 9.96 (s, |
| | | 1H), 9.21 (s, 1H), 8.97 (s, 1H), 8.94 (s, |
| | | 1H), 8.13 (d, J = 5.7 Hz, 1H), 8.09 (d, J = 2.1 |
| | | Hz, 1H), 7.95 (t, J = 9.8 Hz, 1H), |
| | | 7.75 (s, 1H), 7.63 (s, 1H), 6.35 (d, J = 5.7 |
| | | Hz, 1H), 5.54-5.44 (m, 1H), 3.71 |
| | | (s, 3H), 3.19-3.08 (m, 1H), 2.96-2.85 |
| | | (m, 2H), 2.70-2.63 (m, 1H), 2.06- |
| | | 2.00 (m, 1H), 1.79-1.70 (m, 1H). |
| | | 1 proton overlaps with solvent. |
| 264 | 474.5 | 1H NMR (400 MHz, DMSO) δ 8.92 (s, 1H), |
| | | 8.83 (d, J = 2.7 Hz, 1H), 8.47 (d, |
| | | J = 1.9 Hz, 1H), 8.21 (s, 1H), 7.97 (d, J = 2.5 |
| | | Hz, 1H), 7.93 (d, J = 8.2 Hz, 1H), |
| | | 7.88 (dd, J = 8.1, 1.2 Hz, 1H), 7.73 (d, |
| | | J = 5.6 Hz, 1H), 7.62-7.50 (m, 2H), |
| | | 7.37 (d, J = 2.5 Hz, 1H), 7.30 (dd, |
| | | J = 8.7, 2.5 Hz, 1H), 6.75-6.69 (m, 2H), |
| | | 4.29 (p, J = 7.2 Hz, 1H), 3.72 (s, 3H), |
| | | 2.60-2.50 (m, 2H), 2.37-2.26 (m, |
| | | 1H), 2.04 (s, 6H), 1.85-1.73 (m, 2H). |
| 265 | 456.4 | 1H NMR (400 MHz, DMSO) δ 8.99 (s, |
| | | 1H), 8.75 (d, J = 2.6 Hz, 1H), 8.55 (s, |
| | | 1H), 8.19 (s, 1H), 8.02 (d, J = 2.5 Hz, |
| | | 1H), 7.96-7.83 (m, 2H), 7.61-7.50 |
| | | (m, 2H), 7.23 (d, J = 2.4 Hz, 1H), 7.10 (dd, |
| | | J = 8.7, 2.4 Hz, 1H), 6.71 (d, J = |
| | | 8.7 Hz, 1H), 6.53 (d, J = 1.7 Hz, 1H), 6.46 (dd, |
| | | J = 5.8, 1.9 Hz, 1H), 4.26 (p, J = |
| | | 7.2 Hz, 1H), 3.71 (s, 3H), 2.58-2.50 |
| | | (m, 2H), 2.35-2.24 (m, 1H), 2.04 (s, |
| | | 6H), 1.86-1.72 (m, 2H). |
| 266 | 473.4 | 1H NMR (500 MHz, DMSO) δ 11.00 (d, |
| | | J = 5.4 Hz, 1H), 9.69 (s, 1H), 9.05 (s, |
| | | 1H), 8.26 (s, 1H), 8.06 (t, J = 7.6 Hz, 2H), |
| | | 7.57 (d, J = 8.7 Hz, 1H), 7.28-7.21 |
| | | (m, 2H), 7.10-7.07 (m, 1H), 6.76 (d, |
| | | J = 8.7 Hz, 1H), 6.34 (s, 1H), 6.27 (d, J = |
| | | 5.7 Hz, 1H), 4.36-4.30 (m, 1H), |
| | | 3.67 (s, 3H), 2.60-2.54 (m, 2H), 2.34- |
| | | 2.28 (m, 1H), 2.05 (s, 6H), 1.85-1.79 (m, 2H). |
| 267 | 482.3 | 1H NMR (500 MHz, DMSO): δ 9.94 (s, |
| | | 1H), 9.48 (s, 1H), 9.00 (d, J = 2.5 Hz, |
| | | 1H), 8.88 (s, 1H), 8.16 (d, J = 5.7 Hz, 1H), |
| | | 7.94 (d, J = 8.2 Hz, 1H), 7.81 (d, J = |
| | | 7.7 Hz, 1H), 7.74 (s, 1H), 7.66 (d, |
| | | J = 2.4 Hz, 1H), 7.63-7.54 (m, 2H), 6.41 |
| | | (d, J = 5.8 Hz, 1H), 4.88-4.82 (m, 1H), |
| | | 3.69 (s, 3H), 2.97-2.87 (m, 1H), |
| | | 2.23-2.11 (m, 4H), 2.05 (s, 6H). |
| 268 | 493.4 | 1H NMR (500 MHz, DMSO) δ 9.26 (s, |
| | | 1H), 8.96 (s, 1H), 7.97 (d, J = 5.7 Hz, |
| | | 2H), 7.74 (d, J = 2.3 Hz, 1H), 7.29 (d, |
| | | J = 2.1 Hz, 1H), 7.19 (d, J = 8.9 Hz, |
| | | 1H), 6.70 (d, J = 8.7 Hz, 1H), 6.12 (d, |
| | | J = 5.7 Hz, 1H), 4.32-4.27 (m, 1H), |
| | | 4.07 (s, 2H), 3.66 (s, 3H), 2.57-2.53 (m, |
| | | 2H), 2.31-2.27 (m, 1H), 2.04 (s, |
| | | 7H), 1.83-1.77 (m, 2H), 1.32 (s, 6H). |
| 269 | 482.5 | 1H NMR (500 MHz, DMSO) δ 9.76 (s, |
| | | 1H), 9.02 (s, 1H), 8.73 (s, 1H), 8.09 (t, |
| | | J = 7.7 Hz, 2H), 8.00 (d, J = 8.2 Hz, 1H), |
| | | 7.89 (s, 1H), 7.83 (t, J = 7.5 Hz, 1H), |
| | | 7.75 (t, J = 7.3 Hz, 1H), 7.45 (s, 1H), |
| | | 6.36 (d, J = 5.7 Hz, 1H), 4.73-4.64 (m, |
| | | 1H), 3.44 (s, 3H), 2.49-2.43 (m, 2H), |
| | | 2.33-2.27 (m, 1H), 2.03 (s, 6H), 1.77- |
| | | 1.69 (m, 2H). |
| 270 | 476.5 | 1H NMR (500 MHz, DMSO) δ 10.00 (s, |
| | | 1H), 9.19 (s, 1H), 8.98 (s, 2H), 8.16- |
| | | 8.11 (m, 1H), 8.04 (d, J = 1.9 Hz, 1H), |
| | | 7.63-7.49 (m, 3H), 7.42-7.37 (m, |
| | | 1H), 6.35 (d, J = 5.7 Hz, 1H), 4.92-4.83 |
| | | (m, 1H), 3.68 (s, 3H), 2.68-2.62 |
| | | (m, 2H), 2.19 (s, 6H), 1.97-1.88 (m, 2H). |
| 271 | 494.6 | 1H NMR (500 MHz, DMSO) δ 9.31 (s, |
| | | 1H), 9.03 (s, 1H), 7.97 (d, J = 5.7 Hz, |
| | | 1H), 7.91 (d, J = 22.2 Hz, 2H), 7.70 (d, J = 1.8 |
| | | Hz, 1H), 7.60 (s, 1H), 6.14 (d, J = |
| | | 5.8 Hz, 1H), 4.84-4.79 (m, 1H), 4.06 |
| | | (s, 2H), 3.67 (s, 3H), 2.59-2.54 (m, |
| | | 2H), 2.37-2.33 (m, 1H), 2.05 (s, 6H), |
| | | 1.83-1.78 (m, 2H), 1.31 (s, 6H). |
| 272 | 476.4 | 1H NMR (500 MHz, CDCl3) δ 9.98 (s, 1H), |
| | | 9.20 (s, 1H), 9.19 (d, J = 2.5 Hz, |
| | | 1H), 9.01 (s, 1H), 9.00 (d, J = 2.8 Hz, 1H), |
| | | 8.21 (dd, J = 9.5, 2.7 Hz, 1H), 8.12 |
| | | (d, J = 5.6 Hz, 1H), 7.32 (s, 1H), 7.29 (dd, |
| | | J = 8.7, 2.2 Hz, 1H), 6.75 (d, J = 8.7 |
| | | Hz, 1H), 6.33 (d, J = 5.7 Hz, 1H), 4.33 |
| | | (p, J = 6.9 Hz, 1H), 3.65 (s, 3H), 2.58 |
| | | (m, 2H), 2.44-2.36 (m, 1H), 2.10 (s, 6H), |
| | | 1.85 (dd, J = 18.0, 9.6 Hz, 2H). |
| 273 | 493.4 | 1H NMR (500 MHz, DMSO): δ 9.28 (s, |
| | | 1H), 8.98 (s, 1H), 7.98 (s, 1H), 7.97 |
| | | (s, 1H), 7.79 (d, J = 2.2 Hz, 1H), 7.32 (d, |
| | | J = 1.6 Hz, 1H), 7.22 (dd, J = 8.7, 2.3 |
| | | Hz, 1H), 6.83 (d, J = 8.7 Hz, 1H), 6.12 |
| | | (d, J = 5.7 Hz, 1H), 4.38-4.34 (m, |
| | | 2H), 4.30-4.26 (m, 1H), 4.25-4.21 (m, 2H), |
| | | 3.68 (s, 3H), 2.19 (s, 6H), 2.17- |
| | | 2.12 (m, 1H), 1.93-1.86 (m, 2H), |
| | | 1.72-1.62 (m, 2H), 1.54-1.43 (m, 4H). |
| 274 | 476.4 | 1H NMR (500 MHz, DMSO) δ 9.27 (s, 1H), |
| | | 8.21 (d, J = 5.8 Hz, 1H), 7.89 (d, |
| | | J = 2.3 Hz, 1H), 7.68-7.53 (m, 2H), 7.04 |
| | | (d, J = 6.3 Hz, 1H), 6.89 (d, J = 2.4 |
| | | Hz, 1H), 6.75 (d, J = 8.7 Hz, 1H), 6.67 |
| | | (d, J = 5.7 Hz, 1H), 5.66 (s, 2H), 4.32 |
| | | (p, J = 7.0 Hz, 1H), 3.80 (s, 3H), |
| | | 2.61-2.51 (m, 1H), 2.32-2.24 (m, 1H). |
| | | 2.04 (s, 6H), 1.79 (ddd, J = 10.9, |
| | | 8.6, 2.7 Hz, 2H). |
| 275 | 484.6 | 1H NMR (500 MHz, DMSO) δ 11.74 (s, |
| | | 1H), 9.34 (s, 1H), 9.19 (s, 1H), 8.31 |
| | | (s, 1H), 8.13 (d, J = 5.6 Hz, 1H), 8.05 |
| | | (s, 1H), 7.53 (d, J = 8.9 Hz, 1H), 7.27 (s, |
| | | 1H), 7.17 (d, J = 8.1 Hz, 1H), 6.80 (d, |
| | | J = 8.7 Hz, 1H), 6.29 (d, J = 5.7 Hz, |
| | | 1H), 4.39-4.31 (m, 1H), 3.71 (s, 3H), |
| | | 2.62-2.54 (m, 2H), 2.36-2.28 (m, |
| | | 1H), 2.05 (d, J = 5.6 Hz, 6H), |
| | | 1.85-1.77 (m, 2H). |
| 276 | 466.5 | 1H NMR (500 MHz, DMSO) δ 9.31 (s, |
| | | 1H), 9.04 (s, 1H), 7.98 (d, J = 5.7 Hz, |
| | | 2H), 7.93 (s, 1H), 7.70 (s, 1H), 7.63 (s, |
| | | 1H), 6.14 (d, J = 5.8 Hz, 1H), 4.84- |
| | | 4.78 (m, 1H), 4.37-4.33 (m, 2H), |
| | | 4.25-4.22 (m, 2H), 3.68 (s, 3H), 2.59- |
| | | 2.54 (m, 2H), 2.37-2.31 (m, 1H), 2.05 |
| | | (s, 6H), 1.84-1.77 (m, 2H). |
| 277 | 487.4 | 1H NMR (500 MHz, DMSO) δ 9.76 (s, 1H), |
| | | 9.07 (s, 1H), 8.89 (s, 1H), 8.72 (s, |
| | | 1H), 8.07 (d, J = 5.7 Hz, 1H), 7.80 |
| | | (d, J = 9.1 Hz, 1H), 7.34 (s, 1H), 7.21- |
| | | 7.19 (m, 1H), 7.07 (s, 1H), 6.99 (s, 1H), 6.76 |
| | | (d, J = 8.6 Hz, 1H), 6.29 (d, J = |
| | | 5.7 Hz, 1H), 4.35-4.31 (m, 1H), 3.91 |
| | | (s, 3H), 3.60 (s, 3H), 2.60-2.55 (m, |
| | | 2H), 2.32 (s, 1H), 2.07 (s, 6H), 1.83-1.77 (m, 2H) |
| 278 | 492.6, | 1H NMR (500 MHz, DMSO) δ 10.05 (s, |
| | 494.7 | 1H), 9.23 (s, 1H), 9.17 (d, J = 2.5 Hz, |
| | | 1H), 9.03 (s, 1H), 8.92 (d, J = 2.4 Hz, 1H), |
| | | 8.49-8.46 (m, 1H), 8.13 (d, J = 5.6 |
| | | Hz, 1H), 7.34-7.27 (m, 2H), 6.76 (d, J = 8.6 |
| | | Hz, 1H), 6.34 (d, J = 5.7 Hz, |
| | | 1H), 4.35-4.31 (m, 1H), 3.66 (s, 3H), |
| | | 2.59-2.53 (m, 2H), 2.31 (d, J = |
| | | 7.9 Hz, 1H), 2.05 (s, 6H), 1.86-1.79 (m, 2H). |
| 279 | 493.5, | 1H NMR (500 MHz, DMSO) δ 10.11 (s, |
| | 496.0 | 1H), 9.32 (d, J = 9.7 Hz, 1H), 9.17 (d, |
| | | J = 2.5 Hz, 1H), 8.97 (s, 1H), 8.92 (d, |
| | | J = 2.4 Hz, 1H), 8.49-8.46 (m, 1H), |
| | | 8.16-8.13 (m, 1H), 8.00 (s, 1H), 7.67 (s, 1H), |
| | | 6.37 (d, J = 5.7 Hz, 1H), 4.86- |
| | | 4.79 (m, 1H), 3.67 (s, 3H), 2.60-2.54 |
| | | (m, 2H), 2.38-2.32 (m, 1H), 2.05 (s, |
| | | 7H), 1.85-1.79 (m, 2H). |
| 280 | 461.4 | 1H NMR (500 MHz, DMSO) δ 9.96 (s, |
| | | 1H), 9.41 (s, 1H), 9.14-9.09 (m, 2H), |
| | | 8.48-8.43 (m, 2H), 7.74 (s, 1H), 7.63 (d, J = 7.1 |
| | | Hz, 1H), 7.14 (d, J = 8.7 Hz, |
| | | 1H), 6.64 (d, J = 5.7 Hz, 1H), 4.79-4.72 |
| | | (m, 1H), 4.51 (s, 3H), 4.05 (s, 3H), |
| | | 3.03-2.97 (m, 2H), 2.79-2.72 (m, 1H), 2.50 |
| | | (s, 6H), 2.29-2.22 (m, 2H). |
| 281 | 460.4 | 1H NMR (500 MHz, DMSO) δ 11.18 (s, |
| | | 1H), 9.15 (s, 1H), 8.86 (s, 1H), 8.26 |
| | | (d, J = 2.1 Hz, 1H), 8.12 (s, 1H), 7.94 (d, |
| | | J = 5.7 Hz, 1H), 7.31 (s, 1H), 7.21 (d, |
| | | J = 0.8 Hz, 1H), 7.18-7.14 (m, 1H), 6.58 (d, |
| | | J = 8.7 Hz, 1H), 6.11 (d, J = 5.7 |
| | | Hz, 1H), 4.27-4.21 (m, 1H), 3.50 (s, 3H), |
| | | 2.54-2.51 (m, 2H), 2.31-2.25 |
| | | (m, 1H), 2.18 (s, 3H), 2.04 (s, 6H), |
| | | 1.80-1.74 (m, 2H). |
| 282 | 460.4 | 1H NMR (500 MHz, DMSO) δ 9.25 (s, 1H), |
| | | 8.89 (s, 1H), 8.35 (s, 2H), 7.95 (d, |
| | | J = 5.7 Hz, 1H), 7.49 (d, J = 3.4 Hz, 1H), |
| | | 7.31 (s, 1H), 7.18 (d, J = 8.3 Hz, 1H), |
| | | 6.65 (d, J = 8.7 Hz, 1H), 6.37 (d, J = 3.4 |
| | | Hz, 1H), 6.12 (d, J = 5.7 Hz, 1H), 4.31 |
| | | -4.24 (m, 1H), 3.81 (s, 3H), 3.55 (s, |
| | | 3H), 2.58-2.52 (m, 2H), 2.33-2.26 (m, |
| | | 1H), 2.04 (s, 6H), 1.83-1.75 (m, 2H). |
| 283 | 460.4 | 1H NMR (500 MHz, DMSO) δ 11.37 (s, 1H), |
| | | 9.13 (s, 1H), 8.86 (s, 1H), 8.13 |
| | | (s, 2H), 7.93 (d, J = 5.7 Hz, 1H), 7.28 |
| | | (s, 1H), 7.23-7.17 (m, 1H), 6.62 (d, J = |
| | | 8.7 Hz, 1H), 6.12-6.05 (m, 2H), |
| | | 4.30-4.24 (m, 1H), 3.56 (s, 3H), 2.57- |
| | | 2.52 (m, 2H), 2.39 (s, 3H), 2.32-2.26 |
| | | (m, 1H), 2.04 (s, 6H), 1.82-1.76 (m, |
| | | 2H). |
| 284 | 450.4 | 1H NMR (500 MHz, DMSO) δ 11.86 (s, 1H), |
| | | 9.28 (s, 1H), 9.16 (s, 1H), 8.47 |
| | | (s, 1H), 8.22 (dd, J = 4.4, 1.4 Hz, 1H), |
| | | 8.11 (d, J = 5.6 Hz, 1H), 7.95 (s, 1H), |
| | | 7.50 (dd, J = 8.7, 4.4 Hz, 1H), 7.31 (s, 1H), |
| | | 7.19 (d, J = 8.5 Hz, 1H), 6.78 (d, J = |
| | | 8.7 Hz, 1H), 6.27 (d, J = 5.7 Hz, 1H), |
| | | 4.39-4.30 (m, 1H), 3.71 (s, 3H), 2.62- |
| | | 2.54 (m, 2H), 2.33 (s, 1H), 2.06 (s, |
| | | 5H), 1.82 (d, J = 10.2 Hz, 1H). |
| 285 | 492.5, | 1H NMR (400 MHz, DMSO) δ 10.08 (s, 1H), |
| | 494.6 | 9.26 (s, 1H), 9.10 (d, J = 2.5 Hz, |
| | | 1H), 8.98 (s, 1H), 8.38-8.33 (m, 1H), 8.14 |
| | | (d, J = 5.6 Hz, 1H), 7.64 (d, J = 8.7 |
| | | Hz, 1H), 7.36-7.29 (m, 2H), 6.69 (d, |
| | | J = 8.6 Hz, 1H), 6.35 (d, J = 5.7 Hz, |
| | | 1H), 4.71-4.66 (m, 1H), 3.69 (s, 3H), |
| | | 2.82-2.78 (m, 1H), 2.31-2.23 (m, |
| | | 2H), 2.21-2.01 (m, 8H). |
| 286 | 519.4 | 1H NMR (400 MHz, DMSO): δ 9.93 (s, 1H), |
| | | 9.10 (s, 1H), 9.03 (s, 1H), 8.93 |
| | | (d, J = 2.5 Hz, 1H), 8.11 (d, J = 5.7 Hz, |
| | | 1H), 7.94 (d, J = 8.9 Hz, 1H), 7.87 (s, |
| | | 1H), 7.58 (dd, J = 8.9, 2.4 Hz, 1H), 7.32 |
| | | (s, 1H), 7.25 (dd, J = 8.5, 2.2 Hz, 1H), |
| | | 6.91 (d, J = 8.7 Hz, 1H), 6.32 (d, |
| | | J = 5.7 Hz, 1H), 4.34 (s, 1H), 3.68 (s, 3H), |
| | | 2.24-2.10 (m, 7H), 1.98-1.84 (m, 2H), |
| | | 1.76-1.64 (m, 2H), 1.57-1.44 (m, |
| | | 4H). |
| 288 | 465.3 | 1H NMR (500 MHz, DMSO) δ 9.06 (s, 1H), |
| | | 8.61 (s, 1H), 8.46 (s, 1H), 7.81- |
| | | 7.76 (m, 1H), 7.69-7.64 (m, 1H), 7.61 |
| | | (s, 1H), 6.66 (d, J = 8.4 Hz, 1H), 6.28 |
| | | (d, J = 2.4 Hz, 1H), 6.09-6.04 (m, 1H), |
| | | 4.93 (s, 2H), 4.60 (s, 1H), 4.12-4.02 |
| | | (m, 2H), 3.77-3.72 (m, 1H), 3.64 (s, 3H), |
| | | 3.54-3.50 (m, 1H), 3.14 (s, 3H). |
| 289 | 494.3 | 1H NMR (500 MHz, DMSO) δ 9.94 (s, 1H), |
| | | 9.12 (s, 1H), 8.94 (s, 2H), 8.11 (d, |
| | | J = 5.7 Hz, 1H), 8.06 (d, J = 2.2 Hz, |
| | | 1H), 7.97-7.90 (m, 1H), 7.76-7.66 (m, |
| | | 1H), 7.59-7.48 (m, 1H), 6.32 (d, |
| | | J = 5.7 Hz, 1H), 4.91-4.84 (m, 1H), 3.68 |
| | | (s, 3H), 2.61-2.54 (m, 2H), 2.38-2.33 |
| | | (m, 1H), 2.05 (s, 6H), 1.87-1.78 (m, |
| | | 2H) |
| 290 | 494.3, | 1HNMR(500 MHz, DMSO) δ 10.06 (s, 1H), |
| | 495.5 | 9.16 (s, 1H), 9.02 (s, 1H), 8.92 (s, |
| | | 1H), 8.13 (d, J = 5.7 Hz, 1H), 8.07 |
| | | (d, J = 2.2 Hz, 1H), 7.54-7.48 (m, 2H), |
| | | 7.36 (s, 1H), 6.35 (d, J = 5.7 Hz, 1H), |
| | | 4.92-4.86 (m, 1H), 3.70 (s, 3H), 2.62- |
| | | 2.56 (m, 2H), 2.37-2.34 (m, 1H), |
| | | 2.06 (s, 6H), 1.88-1.81 (m, 2H). |
| 291 | 494.4 | 1HNMR(500 MHz, DMSO) δ 9.99 (s, 1H), |
| | | 9.17 (s, 1H), 9.02 (d, J = 22.2 Hz, |
| | | 2H), 8.11 (d, J = 5.7 Hz, 1H), 8.03 |
| | | (d, J = 2.0 Hz, 1H), 7.70-7.63 (m, 2H), |
| | | 7.56 (s, 1H), 6.34 (d, J = 5.7 Hz, 1H), |
| | | 4.87-4.82 (m, 1H), 3.68 (s, 3H), 2.61- |
| | | 2.55 (m, 2H), 2.39-2.34 (m, 1H), |
| | | 2.06 (s, 6H), 1.87-1.79 (m, 2H) |
| 292 | 508.2, | 1HNMR(500 MHz, DMSO) δ 9.01 (s, 1H), |
| | 510.0 | 8.97 (s, 1H), 8.69-8.64 (m, 1H), |
| | | 8.61 (s, 1H), 8.35 (s, 1H), 7.66-7.58 (m, |
| | | 1H), 6.66 (d, J = 8.4 Hz, 1H), 6.28 |
| | | (d, J = 2.4 Hz, 1H), 6.08-6.05 (m, |
| | | 1H), 4.93 (s, 1H), 4.61 (s, 1H), 4.09 (s, |
| | | 2H), 3.82-3.70 (m, 2H), 3.64 (s, 3H), |
| | | 3.54-3.50 (m, 1H), 3.17 (s, 3H). |
| 293 | 463.4, | 1HNMR(400 MHz, dmso) δ 9.09 (s, 1H), |
| | 465.6 | 8.66 (s, 1H), 8.08 (d, J = 2.3 Hz, |
| | | 1H), 7.98 (d, J = 8.9 Hz, 1H), |
| | | 7.82-7.74 (m, 1H), 7.68-7.62 (m, 1H), 6.68 |
| | | (d, J = 8.4 Hz, 1H), 6.55 (s, 1H), 6.28 |
| | | (d, J = 2.4 Hz, 1H), 6.10-6.05 (m, 1H), |
| | | 4.99-4.83 (m, 1H), 4.64 (s, 1H), 4.18-4.09 |
| | | (m, 2H), 3.79 (d, J = 11.7 Hz, |
| | | 1H), 3.64 (s, 3H), 3.57 |
| | | (d, J = 13.8 Hz, 2H), 3.22 (s, 3H). |
| 294 | 433.6, | 1HNMR(400 MHz, dmso) δ 9.05 (s, 1H), |
| | 435.8 | 8.62 (s, 1H), 8.44 (s, 1H), 8.07 (d, J = |
| | | 2.3 Hz, 1H), 7.97 (d, J = 8.9 Hz, 1H), |
| | | 7.74 (d, J = 7.1 Hz, 1H), 7.65-7.61 |
| | | (m, 1H), 6.75-6.70 (m, 2H), 6.55-6.50 |
| | | (m, 2H), 6.47 (s, 1H), 4.89-4.85 |
| | | (m, 1H), 4.23-4.12 (m, 2H), 3.82 (d, J = 11.3 |
| | | Hz, 1H), 3.58 (d, J = 13.7 Hz, |
| | | 2H), 3.20 (s, 3H). |
| 295 | 399.6 | 1HNMR(400 MHz, dmso) δ 9.04 (s, 1H), |
| | | 8.63 (s, 1H), 8.47 (s, 2H), 8.03- |
| | | 7.86 (m, 2H), 7.81-7.50 (m, 3H), |
| | | 6.74-6.71 (m, 1H), 6.54-6.51 (m, 1H), |
| | | 6.44 (s, 1H), 4.86 (s, 1H), 4.17 (s, 2H), 3.82 |
| | | (d, J = 13.6 Hz, 1H), 3.61-3.49 |
| | | (m, 2H), 3.19 (s, 3H). |
| 296 | 501.6 | 1H NMR (500 MHz, DMSO) δ 13.36 (s, 1H), |
| | | 9.36 (s, 1H), 9.06 (s, 1H), 8.05 |
| | | (d, J = 5.7 Hz, 1H), 7.96 (d, J = 8.0 Hz, 1H), |
| | | 7.67 (s, 1H), 7.55 (t, J = 7.0 Hz, |
| | | 1H), 7.51 (d, J = 7.2 Hz, 1H), 7.32 (s, 1H), |
| | | 7.27 (d, J = 8.7 Hz, 1H), 6.79 (d, J = |
| | | 8.7 Hz, 1H), 6.20 (d, J = 5.7 Hz, 1H), |
| | | 4.40 (d, J = 6.4 Hz, 1H), 4.09 (d, J = |
| | | 5.6 Hz, 1H), 3.67 (s, 3H), 3.18 (d, |
| | | J = 4.8 Hz, 4H), 2.69 (s, 2H), 2.32 (s, 6H), |
| | | 2.03 (s, 2H). |
| 297 | 499.6 | 1H NMR (500 MHz, DMSO) δ 11.82 (s, 1H), |
| | | 9.63 (s, 1H), 9.17 (s, 1H), 8.76 |
| | | (s, 1H), 8.17 (s, 1H), 8.13 (d, J = 5.6 Hz, 1H), |
| | | 8.01 (d, J = 8.5 Hz, 1H), 7.77 (s, |
| | | 1H), 7.69 (ddd, J = 8.4, 6.8, 1.5 Hz, 1H), |
| | | 7.67-7.60 (m, 1H), 7.28 (s, 1H), |
| | | 7.23 (d, J = 8.2 Hz, 1H), 6.80 (d, |
| | | J = 8.7 Hz, 1H), 6.32 (d, J = 5.7 Hz, 1H), 4.41- |
| | | 4.30 (m, 1H), 3.68 (s, 3H), 2.62 (dd, |
| | | J = 18.1, 4.2 Hz, 2H), 2.43-2.32 (m, |
| | | 1H), 2.10 (s, 6H), 1.87 (d, J = 8.2 Hz, 2H). |
| 298 | 493.3 | 1HNMR(500 MHz, DMSO) δ 9.95 (s, 1H), |
| | | 9.10 (s, 1H), 9.05 (s, 2H), 8.10 (d, |
| | | J = 5.7 Hz, 1H), 7.71-7.59 (m, 2H), |
| | | 7.27 (s, 1H), 7.21 (d, J = 8.4 Hz, 1H), |
| | | 6.75 (d, J = 8.7 Hz, 1H), 6.31 (d, |
| | | J = 5.7 Hz, 1H), 4.36-4.30 (m, 1H), 3.65 (s, |
| | | 3H), 2.59-2.54 (m, 2H), 2.34-2.29 |
| | | (m, 1H), 2.04 (s, 6H), 1.85-1.78 (m, |
| | | 2H). |
| 299 | 446.6 | 1HNMR(500 MHz, DMSO) δ 11.11 (s, 1H), |
| | | 9.27 (s, 1H), 8.90 (s, 1H), 8.46 (d, |
| | | J = 2.1 Hz, 1H), 8.02 (s, 1H), 7.97 |
| | | (d, J = 5.7 Hz, 1H), 7.54-7.52 (m, 1H), |
| | | 7.36 (d, J = 2.4 Hz, 1H), 7.22-7.19 |
| | | (m, 1H), 6.63 (d, J = 8.7 Hz, 1H), 6.50 (d, |
| | | J = 2.1 Hz, 1H), 6.15 (d, J = 5.7 Hz, |
| | | 1H), 4.29-4.23 (m, 1H), 3.52 (s, 3H), |
| | | 2.55-2.51 (m, 2H), 2.32-2.26 (m, 1H), |
| | | 2.04 (s, 7H), 1.81-1.74 (m, 2H). |
| 300 | 447.5 | 1HNMR(500 MHz, DMSO) δ 13.54 (s, 1H), |
| | | 9.46 (s, 1H), 8.95 (s, 1H), 8.65 (s, |
| | | 1H), 8.58 (d, J = 2.3 Hz, 1H), 8.00 (d, |
| | | J = 5.7 Hz, 2H), 7.29 (s, 1H), 7.17 (d, J = |
| | | 8.6 Hz, 1H), 6.68 (d, J = 8.7 Hz, 1H), |
| | | 6.18 (d, J = 5.7 Hz, 1H), 4.33-4.27 |
| | | (m, 1H), 3.59 (s, 3H), 2.59-2.53 (m, 2H), |
| | | 2.32 (s, 1H), 2.06 (s, 6H), 1.86- |
| | | 1.77 (m, 2H). |
| 301 | 446.6 | 1HNMR(500 MHz, DMSO) δ 11.53 (s, 1H), |
| | | 9.19 (s, 1H), 8.86 (s, 1H), 8.32- |
| | | 8.26 (m, 2H), 7.94 (d, J = 5.7 Hz, |
| | | 1H), 7.45-7.42 (m, 1H), 7.32 (s, 1H), 7.17 |
| | | (d, J = 8.8 Hz, 1H), 6.64 (d, J = 8.7 |
| | | Hz, 1H), 6.40-6.33 (m, 1H), 6.12 (d, J = |
| | | 5.7 Hz, 1H), 4.31-4.25 (m, 1H), 3.55 |
| | | (s, 3H), 2.56-2.51 (m, 2H), 2.34- |
| | | 2.28 (m, 1H), 2.06 (s, 6H), 1.83-1.77 (m, 2H). |
| 302 | 481.3 | 1HNMR(400 MHz, DMSO) δ 10.46 (s, 1H), |
| | | 9.16 (s, 2H), 9.05 (d, J = 2.6 Hz, |
| | | 1H), 8.07 (d, J = 5.7 Hz, 1H), 8.01 (s, |
| | | 1H), 7.89 (d, J = 8.6 Hz, 1H), 7.59- |
| | | 7.56 (m, 1H), 7.30 (d, J = 6.7 Hz, 2H), 6.75 |
| | | (d, J = 9.0 Hz, 1H), 6.46 (d, J = 5.7 |
| | | Hz, 1H), 4.37 (s, 1H), 4.37-4.32 (m, 1H), |
| | | 3.68 (d, J = 6.5 Hz, 4H), 2.58 (s, |
| | | 2H), 2.08 (s, 6H), 1.90 (s, 2H) |
| 303 | 547.5 | 1H NMR (500 MHz, CDCl3) δ 9.37 (s, 1H), |
| | | 8.96 (s, 1H), 8.46 (s, 1H), 8.00 (d, |
| | | J = 5.7 Hz, 1H), 7.96 (d, J = 1.8 Hz, 1H), |
| | | 7.27 (s, 1H), 7.17 (d, J = 8.7 Hz, 1H), |
| | | 6.85 (d, J = 8.8 Hz, 1H), 6.17 (d, |
| | | J = 5.8 Hz, 1H), 3.97-3.91 (m, 6H), 3.65 (s, |
| | | 3H), 2.90 (t, J = 6.9 Hz, 2H), 2.87 (s, |
| | | 2H), 2.5 (broad singlet overlapping with |
| | | solvent peak, 6H), 1.95 (t, J = 6.8 Hz, |
| | | 2H), 1.90 (s, 2H), 1.55 (s, 4H), 1.42 (s, |
| | | 2H). |
| 304 | 489.4 | 1H NMR (400 MHz, DMSO): δ 10.26 (s, 1H), |
| | | 9.15 (s, 1H), 9.01 (s, 1H), 8.99 |
| | | (s, 1H), 8.16 (d, J = 5.6 Hz, 1H), 8.09 (s, 1H), |
| | | 7.24 (s, 1H), 7.17 (d, J = 8.6 Hz, |
| | | 1H), 6.81 (d, J = 8.7 Hz, 1H), 6.34 (d, |
| | | J = 5.6 Hz, 1H), 4.35 (dd, J = 14.2, 7.2 |
| | | Hz, 1H), 3.72 (s, 3H), 3.69 (s, 3H), |
| | | 2.62-2.55 (m, 2H), 2.37-2.27 (m, 1H), |
| | | 2.06 (s, 6H), 1.89-1.79 (m, 2H). |
| 305 | 487.2 | 1H NMR (500 MHz, DMSO) δ 9.76 (s, 1H), |
| | | 9.06 (s, 1H), 8.89 (s, 1H), 8.72 (s, |
| | | 1H), 8.07 (d, J = 5.7 Hz, 1H), 7.80 |
| | | (d, J = 9.1 Hz, 1H), 7.34 (s, 1H), 7.21- |
| | | 7.18 (m, 1H), 7.00 (s, 2H), 6.67 (d, J = 8.7 |
| | | Hz, 1H), 6.29 (d, J = 5.7 Hz, 1H), |
| | | 4.66-4.62 (m, 1H), 3.91 (s, 3H), 3.61 |
| | | (s, 3H), 2.81-2.78 (m, 1H), 2.31- |
| | | 2.26 (m, 2H), 2.13-2.09 (m, 2H), 2.07 (s, 6H). |
| 306 | 492.6, | 1H NMR (500 MHz, Acetone) δ 9.59 (s, 1H), |
| | 494.4 | 8.36 (d, J = 5.6 Hz, 1H), 8.13 (d, |
| | | J = 5.6 Hz, 1H), 7.92-7.88 (m, 1H), |
| | | 7.84 (d, J = 8.5 Hz, 1H), 7.62-7.60 (m, |
| | | 1H), 7.51 (d, J = 2.4 Hz, 1H), 7.46-7.43 |
| | | (m, 1H), 6.79 (d, J = 8.7 Hz, 1H), |
| | | 4.42-4.36 (m, 1H), 3.84-3.76 (m, 3H), |
| | | 2.65-2.60 (m, 2H), 2.18-2.16 (m, |
| | | 2H), 2.13 (s, 6H), 2.07-2.07 (m, 1H), |
| | | 1.98-1.92 (m, 2H). |
| 307 | 536.4 | 1H NMR (500 MHz, Acetone) δ 9.42 (s, 1H), |
| | | 8.37 (d, J = 5.7 Hz, 1H), 8.16 (d, |
| | | J = 5.7 Hz, 1H), 8.10 (d, J = 1.8 Hz, 1H), |
| | | 8.01 (d, J = 8.6 Hz, 1H), 7.70-7.68 |
| | | (m, 1H), 7.54-7.53 (m, 1H), 7.48-7.45 |
| | | (m, 1H), 6.81 (d, J = 8.7 Hz, 1H), |
| | | 4.39 (d, J = 7.5 Hz, 1H), 3.86 (s, 3H), |
| | | 2.65-2.61 (m, 2H), 2.20-2.19 (m, |
| | | 1H), 2.13-2.09 (m, 8H), 1.95-1.94 (m, 2H). |
| 308 | 544.6 | 1H NMR (400 MHz, DMSO): δ 9.92 (s, 1H), |
| | | 9.08 (s, 1H), 9.01 (s, 1H), 8.91 |
| | | (d, J = 2.4 Hz, 1H), 8.09 (d, J = 5.7 Hz, |
| | | 1H), 7.94 (d, J = 8.9 Hz, 1H), 7.85 (s, |
| | | 1H), 7.71 (s, 1H), 7.58 (dd, J = 8.9, |
| | | 2.4 Hz, 2H), 7.55 (s, 1H), 6.97-6.88 (m, |
| | | 2H), 6.30 (d, J = 5.7 Hz, 1H), 4.49-4.45 |
| | | (m, 1H), 3.16-3.10 (m, 2H), 3.04 |
| | | (s, 2H), 2.67-2.61 (m, 2H), 2.40-2.31 |
| | | (m, 1H), 1.99-1.90 (m, 2H), 1.71- |
| | | 1.52 (m, 6H). |
| 309 | 580.3 | 1H NMR (400 MHz, DMSO): δ 9.91 (s, 1H), |
| | | 9.09 (s, 1H), 9.01 (s, 1H), 8.91 |
| | | (d, J = 2.5 Hz, 1H), 8.09 (d, J = 5.7 Hz, 1H), |
| | | 7.94 (d, J = 8.9 Hz, 1H), 7.86 (s, |
| | | 1H), 7.58 (dd, J = 8.9, 2.4 Hz, 2H), 7.55 (s, |
| | | 1H), 6.94-6.88 (m, 2H), 6.30 (d, J = |
| | | 5.7 Hz, 1H), 4.49 (ddd, J = 4.2, 2.7, 1.7 |
| | | Hz, 1H), 3.07-3.03 (m, 4H), 2.99 |
| | | (dd, J = 6.6, 2.6 Hz, 4H), 2.62 (ddd, |
| | | J = 13.2, 6.8, 3.1 Hz, 1H), 1.99 (ddt, J = |
| | | 6.4, 5.0, 3.0 Hz, 2H), 1.69-1.50 (m, 6H). |
| 310 | 489.6 | 1H NMR (500 MHz, DMSO): δ 9.91 (s, 1H), |
| | | 9.08 (s, 1H), 9.02 (s, 1H), 8.91 |
| | | (d, J = 2.4 Hz, 1H), 8.09 (d, J = 5.6 Hz, |
| | | 1H), 7.94 (d, J = 8.9 Hz, 1H), 7.86 (s, |
| | | 1H), 7.58 (dd, J = 8.9, 2.4 Hz, 2H), 7.55 |
| | | (s, 1H), 6.92-6.88 (m, 2H), 6.30 (d, J = |
| | | 5.7 Hz, 1H), 4.47-4.42 (m, 1H), |
| | | 2.18 (s, 6H), 2.16-2.11 (m, 1H), 1.97- |
| | | 1.89 (m, 2H), 1.66-1.51 (m, 6H). |
| 311 | 531.3 | 1H NMR (500 MHz, DMSO): δ 9.92 (s, 1H), |
| | | 9.08 (s, 1H), 9.02 (s, 1H), 8.91 |
| | | (d, J = 2.4 Hz, 1H), 8.09 (d, J = 5.7 Hz, |
| | | 1H), 7.94 (d, J = 8.9 Hz, 1H), 7.85 (s, |
| | | 1H), 7.58 (dd, J = 8.9, 2.4 Hz, 2H), 7.55 |
| | | (s, 1H), 6.94-6.86 (m, 2H), 6.30 (d, J = |
| | | 5.7 Hz, 1H), 4.49-4.44 (m, 1H), 3.60-3.55 |
| | | (m, 4H), 2.48-2.45 (m, 4H), |
| | | 2.26-2.17 (m, 1H), 1.94 (ddt, J = 10.1, 5.2, |
| | | 3.5 Hz, 2H), 1.71-1.52 (m, 6H). |
| 312 | 487.3 | 1H NMR (500 MHz, DMSO) δ 9.82 (s, 1H), |
| | | 9.08 (s, 1H), 8.93 (s, 1H), 8.85 (s, |
| | | 1H), 8.08 (d, J = 5.7 Hz, 1H), 7.44 (t, |
| | | J = 7.9 Hz, 1H), 7.32-7.19 (m, 3H), |
| | | 7.08-6.96 (m, 1H), 6.77 (t, J = 8.4 Hz, 1H), |
| | | 6.30 (d, J = 5.7 Hz, 1H), 4.35 (p, |
| | | J = 7.3 Hz, 1H), 3.95 (s, 3H), 3.65 (s, 3H), |
| | | 2.59 (m, 2H), 2.35 (dd, J = 15.4, 8.2 |
| | | Hz, 1H), 2.07 (s, 6H), 1.85 (dd, |
| | | J = 19.5, 8.3 Hz, 2H). |
| 313 | 482.3 | 1H NMR (500 MHz, DMSO) δ 10.04 (s, 1H), |
| | | 9.12 (s, 1H), 9.12-9.06 (m, |
| | | 2H), 8.32 (s, 1H), 8.13 (d, J = 5.6 Hz, 1H), |
| | | 8.08 (d, J = 8.6 Hz, 1H), 7.88 (dd, J = |
| | | 8.6, 1.9 Hz, 1H), 7.26 (s, 2H), 7.24 (s, 1H), |
| | | 6.79 (d, J = 8.5 Hz, 1H), 6.33 (d, |
| | | J = 5.7 Hz, 1H), 4.40-4.32 (m, 1H), |
| | | 3.68 (s, 3H), 2.58 (m, 2H), 2.39-2.31 |
| | | (m, 1H), 2.06 (s, 6H), 1.84 (m, 2H). |
| 314 | 536.4, | 1H NMR (500 MHz, DMSO) δ 10.41 (s, 1H), |
| | 538.4 | 9.11 (s, 1H), 8.85 (d, J = 2.2 Hz, |
| | | 1H), 8.48 (d, J = 2.2 Hz, 1H), 8.28-8.20 |
| | | (m, 3H), 7.50 (d, J = 5.2 Hz, 1H), |
| | | 7.36-7.30 (m, 2H), 6.77 (d, J = 8.5 Hz, 1H), |
| | | 4.34 (dddd, J = 7.1 Hz, 1H), 3.75 |
| | | (s, 3H), 2.60-2.54 (m, 2H), 2.34-2.28 |
| | | (m, 1H), 2.05 (s, 6H), 1.86-1.76 (m, |
| | | 2H) |
| 315 | 431.7 | 1H NMR (500 MHz, DMSO) δ 9.30 (s, 1H), |
| | | 8.95 (s, 1H), 8.39 (s, 1H), 7.97 (d, |
| | | J = 5.7 Hz, 1H), 7.92 (s, 1H), 7.50 |
| | | (d, J = 8.9 Hz, 1H), 6.75-6.69 (m, 2H), |
| | | 6.14 (d, J = 5.7 Hz, 1H), 4.67-4.64 |
| | | (m, 1H), 2.82-2.78 (m, 1H), 2.77-2.73 |
| | | (m, 2H), 2.69-2.63 (m, 2H), 2.38-2.29 |
| | | (m, 3H), 2.15-1.96 (m, 8H), 1.86- |
| | | 1.79 (m, 2H), 1.76-1.71 (m, 2H). |
| 316 | 536.4, | 1H NMR (500 MHz, DMSO) δ 10.08 (s, 1H), |
| | 538.1 | 9.22 (s, 1H), 9.15 (d, J = 2.5 Hz, |
| | | 1H), 9.01 (s, 1H), 8.97 (d, J = 2.3 Hz, |
| | | 1H), 8.61-8.60 (m, 1H), 8.14-8.12 |
| | | (m, 1H), 7.31-7.28 (m, 2H), 6.76 (d, J = 8.5 |
| | | Hz, 1H), 6.34 (d, J = 5.6 Hz, |
| | | 1H), 4.35-4.31 (m, 1H), 3.66 (s, 3H), |
| | | 2.58-2.54 (m, 2H), 2.33-2.29 (m, |
| | | 1H), 2.05 (s, 6H), 1.83-1.79 (m, 2H). |
| 317 | 435.4 | 1H NMR (500 MHz, DMSO) δ 9.26 (s, 1H), |
| | | 8.97 (s, 1H), 7.95 (d, J = 5.7 Hz, |
| | | 2H), 7.79 (d, J = 2.3 Hz, 1H), 7.54 (d, |
| | | J = 9.0 Hz, 2H), 6.72-6.69 (m, 2H), |
| | | 6.10 (d, J = 5.7 Hz, 1H), 4.67-4.64 |
| | | (m, 1H), 4.38-4.36 (m, 2H), 4.27-4.24 |
| | | (m, 2H), 2.83-2.77 (m, 1H), 2.35-2.28 |
| | | (m, 2H), 2.14-2.10 (m, 2H), 2.09- |
| | | 2.05 (m, 6H). |
| 318 | 449.4 | 1H NMR (500 MHz, DMSO) δ 9.35 (s, |
| | | 1H), 9.02 (s, 1H), 8.31 (s, 1H), 8.00- |
| | | 7.97 (m, 2H), 7.51 (d, J = 8.9 Hz, 2H), |
| | | 6.78-6.74 (m, 2H), 6.15 (d, J = 5.7 Hz, |
| | | 1H), 4.69-4.65 (m, 1H), 3.29 (s, 1H), |
| | | 3.05-3.01 (m, 2H), 2.86-2.76 (m, |
| | | 3H), 2.35-2.28 (m, 2H), 2.16-2.01 (m, 9H). |
| 319 | 461.6, | 1H NMR (500 MHz, MeOD) δ 8.94 (s, 1H), |
| | 463.3 | 8.76 (d, J = 2.4 Hz, 1H), 8.03 (s, |
| | | 1H), 7.91 (d, J = 8.9 Hz, 1H), 7.60-7.49 |
| | | (m, 2H), 7.48-7.39 (m, 2H), 6.86 |
| | | (d, J = 8.9 Hz, 1H), 6.29 (d, J = 5.7 |
| | | Hz, 1H), 4.62 (s, 2H), 3.17-3.14 (m, 1H), |
| | | 2.54-2.48 (m, 2H), 2.47-2.41 (m, 2H), |
| | | 2.39-2.33 (m, 6H), 1.39-1.20 (m, |
| | | 2H). |
| 320 | 507.3, | 1H NMR (500 MHz, MeOD) δ 8.90 (s, |
| | 509.1 | 1H), 8.58 (s, 1H), 8.04 (s, 1H), 7.50 (d, |
| | | J = 8.6 Hz, 1H), 7.46-7.43 (m, 1H), |
| | | 7.26 (s, 1H), 7.20 (s, 1H), 7.07 (s, 1H), |
| | | 6.89-6.87 (m, 1H), 6.81 (d, J = 8.8 Hz, |
| | | 1H), 6.46 (d, J = 5.6 Hz, 1H), 3.79 (s, |
| | | 1H), 2.69 (s, 2H), 2.43-2.41 (m, 1H), |
| | | 2.24-2.23 (m, 2H), 1.32 (s, 6H), 0.93 |
| | | (s, 1H), 0.13-0.09 (m, 1H). |
| 321 | 473.3 | 1H NMR (400 MHz, DMSO) δ 9.27 (s, 1H), |
| | | 8.92 (s, 1H), 8.24 (d, J = 1.9 Hz, |
| | | 1H), 8.02 (d, J = 2.2 Hz, 1H), 7.97 (d, |
| | | J = 5.7 Hz, 1H), 7.26 (d, J = 2.1 Hz, |
| | | 1H), 7.16 (dd, J = 8.6, 2.2 Hz, 1H), 6.70 |
| | | (d, J = 8.7 Hz, 1H), 6.14 (d, J = 5.7 |
| | | Hz, 1H), 4.30 (p, J = 7.2 Hz, 1H), 3.64 |
| | | (s, 3H), 3.35 (s, 1H), 3.25 (s, 1H), 2.60- |
| | | 2.51 (m, 2H), 2.36-2.24 (m, 1H), 2.04 |
| | | (s, 6H), 1.94 (d, J = 8.4 Hz, 2H), |
| | | 1.86-1.75 (m, 2H), 1.73 (d, J = 8.9 Hz, |
| | | 1H), 1.55 (d, J = 8.9 Hz, 1H), 1.19- |
| | | 1.06 (m, 2H). |
| 322 | 519.5 | 1H NMR (400 MHz, DMSO) δ 9.37 (s, 1H), |
| | | 8.94 (s, 1H), 8.44 (s, 1H), 8.01- |
| | | 7.96 (m, 2H), 7.23 (s, 1H), 7.14 (d, J = 8.4 |
| | | Hz, 1H), 6.73 (d, J = 8.7 Hz, 1H), |
| | | 6.16 (d, J = 5.7 Hz, 1H), 4.31 (p, J = 7.2 |
| | | Hz, 1H), 3.97 (m, 4H), 3.63 (s, 3H), |
| | | 2.89 (m, 4H), 2.61-2.52 (m, 2H), 2.36-2.19 |
| | | (m, 1H), 2.04 (s, 6H), 1.95 (t, J = |
| | | 6.8 Hz, 2H), 1.81 (ddd, J = 16.6, 8.5, 2.7 Hz, 2H). |
| 323 | 493.3 | 1H NMR (500 MHz, DMSO) δ 9.89 (s, 1H), |
| | | 9.03 (s, 1H), 8.98 (s, 2H), 8.04 (d, |
| | | J = 5.7 Hz, 1H), 7.60 (m, 2H), 7.21 (s, 1H), |
| | | 7.15 (d, J = 7.9 Hz, 1H), 6.60 (d, J = |
| | | 8.7 Hz, 1H), 6.24 (d, J = 5.7 Hz, 1H), |
| | | 4.59 (m, 1H), 3.60 (s, 3H), 2.77 (m, |
| | | 1H), 2.31-2.17 (m, 2H), 2.15-2.05 |
| | | (m, 2H), 2.04 (s, 6H). |
| 324 | 491.6, | 1H NMR (500 MHz, DMSO) δ 9.91 (s, |
| | 493.5 | 1H), 9.08 (s, 1H), 9.01 (s, 1H), 8.96 |
| | | (d, J = 2.4 Hz, 1H), 8.09 (d, J = 5.7 Hz, |
| | | 1H), 7.97 (d, J = 2.1 Hz, 1H), 7.77 (s, |
| | | 1H), 7.60-7.57 (m, 1H), 7.27 (s, 1H), 7.22 (d, |
| | | J = 9.0 Hz, 1H), 6.68 (d, J = 8.7 |
| | | Hz, 1H), 6.30 (d, J = 5.7 Hz, 1H), |
| | | 4.68-4.65 (m, 1H), 3.65 (s, 3H), 2.84- |
| | | 2.81 (m, 1H), 2.31-2.27 (m, 2H), |
| | | 2.18-2.05 (m, 8H). |
| 325 | 497.4 | 1H NMR (500 MHz, DMSO) δ 9.75 (s, 1H), |
| | | 9.05 (s, 1H), 8.84 (s, 1H), 8.79 (d, |
| | | J = 2.1 Hz, 1H), 8.06 (d, J = 5.7 Hz, 1H), |
| | | 7.79 (d, J = 8.6 Hz, 1H), 7.36 (s, 1H), |
| | | 7.33-7.28 (m, 2H), 7.15 (s, 1H), 6.68-6.65 |
| | | (m,1H), 6.28 (d, J = 5.7 Hz, 1H), |
| | | 4.70-4.65 (m,1H), 3.74-3.60 (m, 3H), |
| | | 2.82-2.78 (m, 1H), 2.32-2.26 (m, |
| | | 2H), 2.17-2.05 (m, 8H), 1.33-1.30 (m, 1H), |
| | | 1.08-1.03 (m, 2H), 0.85-0.82 |
| | | (m, 2H). |
| 326 | 473.3 | 1H NMR (500 MHz, DMSO) δ 9.94 (s, 1H), |
| | | 9.60 (s, 1H), 8.98 (s, 1H), 8.80- |
| | | 8.78 (m, 1H), 8.76 (s, 1H), 8.03 (d, J = 5.7 |
| | | Hz, 1H), 7.62-7.58 (m, 1H), 7.26 |
| | | (s, 1H), 7.22 (d, J = 8.6 Hz, 1H), 7.19 |
| | | (d, J = 2.4 Hz, 1H), 7.13-7.10 (m, 1H), |
| | | 6.63 (d, J = 8.7 Hz, 1H), 6.23 (d, J = 5.7 |
| | | Hz, 1H), 4.67-4.63 (m, 1H), 3.62 (s, |
| | | 3H), 2.84-2.80 (m, 1H), 2.31-2.26 |
| | | (m, 2H), 2.16-2.11 (m, 2H), 2.08 (s, |
| | | 6H). |
| 327 | 487.3 | 1H NMR (500 MHz, DMSO): δ 9.78 (s, 1H), |
| | | 9.12 (d, J = 2.4 Hz, 1H), 9.10 (s, |
| | | 1H), 8.87 (s, 1H), 8.15 (d, J = 3.6 Hz, 1H), |
| | | 7.96 (d, J = 8.3 Hz, 1H), 7.80 (d, J = |
| | | 7.7 Hz, 1H), 7.64 (ddd, J = 8.4, 6.9, 1.5 |
| | | Hz, 1H), 7.60-7.55 (m, 1H), 7.49 |
| | | (d, J = 8.9 Hz, 2H), 6.72-6.67 (m, 2H), |
| | | 4.71-4.63 (m, 1H), 3.61 (t, J = 4.5 |
| | | Hz, 4H), 2.95-2.85 (m, 1H), 2.39-2.27 |
| | | (m, 6H), 2.16-2.08 (m, 2H). |
| 328 | 535.6, | 1H NMR (400 MHz, DMSO) δ 9.92 (s, 1H), |
| | 537.4 | 9.10 (s, 1H), 9.02 (s, 1H), 8.96 (d, |
| | | J = 2.5 Hz, 1H), 8.14 (d, J = 1.0 Hz, 1H), |
| | | 8.10 (d, J = 5.7 Hz, 1H), 7.70 (s, 2H), |
| | | 7.27 (s, 1H), 7.24 (s, 1H), 7.22 (s, 1H), 6.69 |
| | | (d, J = 8.7 Hz, 1H), 6.31 (d, J = |
| | | 5.7 Hz, 1H), 4.70-4.66 (m, 1H), 3.66 |
| | | (s, 3H), 2.85-2.81 (m, 1H), 2.34- |
| | | 2.29 (m, 2H), 2.19-2.13 (m, 2H), 2.08 (s, 6H). |
| 329 | 535.3 | 1H NMR (400 MHz, DMSO) δ 10.06 (s, 1H), |
| | | 9.33 (s, 1H), 9.18 (s, 1H), 9.10 |
| | | (d, J = 2.6 Hz, 1H), 8.44 (d, J = 21.0 Hz, |
| | | 1H), 8.17-8.11 (m, 2H), 8.03-7.99 |
| | | (m, 1H), 7.34 (s, 1H), 7.25 (d, J = 8.3 Hz, |
| | | 1H), 6.69 (d, J = 8.7 Hz, 1H), 6.34 |
| | | (d, J = 5.7 Hz, 1H), 4.70-4.65 (m, 1H), |
| | | 3.65 (s, 3H), 3.33-3.30 (m, 3H), |
| | | 2.84-2.79 (m, 1H), 2.32-2.27 (m, 2H), |
| | | 2.16-2.10 (m, 2H), 2.07 (s, 6H) |
| 330 | 473.4 | 1H NMR (500 MHz, DMSO) δ 9.89-9.83 (m, |
| | | 1H), 9.67 (s, 1H), 9.07 (s, 1H), |
| | | 8.75-8.68 (m, 2H), 8.06 (d, J = 5.7 Hz, 1H), |
| | | 7.75 (d, J = 8.9 Hz, 1H), 7.29 (d, |
| | | J = 6.9 Hz, 2H), 7.15-7.13 (m, 1H), |
| | | 7.02 (s, 1H), 6.63 (d, J = 9.2 Hz, 1H), |
| | | 6.27 (d, J = 5.7 Hz, 1H), 4.66-4.63 |
| | | (m, 1H), 3.66 (s, 3H), 2.82-2.79 (m, |
| | | 1H), 2.30-2.25 (m, 2H), 2.16-2.11 |
| | | (m, 2H), 2.06 (s, 6H). |
| 331 | 485.5 | 1H NMR (400 MHz, DMSO): δ 9.82 (s, 1H), |
| | | 9.10 (s, 1H), 8.97 (s, 1H), 8.93 |
| | | (d, J = 2.6 Hz, 1H), 8.09 (d, J = 5.7 Hz, 1H), |
| | | 7.92 (d, J = 8.4 Hz, 1H), 7.76 (d, J = |
| | | 7.4 Hz, 1H), 7.59 (ddd, J = 8.4, 6.9, |
| | | 1.6 Hz, 1H), 7.56-7.50 (m, 1H), 7.32 |
| | | (d, J = 1.0 Hz, 1H), 7.23 (dd, J = 8.7, 2.2 |
| | | Hz, 1H), 6.31 (d, J = 5.7 Hz, 1H), |
| | | 4.36-4.30 (m, 1H), 3.64 (s, 3H), 2.20 |
| | | (s, 6H), 2.19-2.10 (m, 2H), 1.96- |
| | | 1.87 (m, 2H), 1.76-1.64 (m, 2H), |
| | | 1.58-1.47 (m, 4H). |
| 332 | 519.6 | 1H NMR (400 MHz, DMSO): δ 9.98 (s, 1H), |
| | | 9.15 (s, 1H), 9.09 (d, J = 2.2 Hz, |
| | | 1H), 9.04 (s, 1H), 8.11 (d, J = 5.7 Hz, 1H), |
| | | 7.76 (dd, J = 7.4, 1.1 Hz, 1H), 7.73 |
| | | (s, 1H), 7.50 (t, J = 7.8 Hz, 1H), 7.32 (s, |
| | | 1H), 7.23 (dd, J = 8.3, 2.1 Hz, 1H), |
| | | 6.90 (d, J = 8.7 Hz, 1H), 6.33 (d, |
| | | J = 5.7 Hz, 1H), 4.37-4.31 (m, 1H), 3.66 (s, |
| | | 3H), 2.21-2.18 (m, 6H), 2.17-2.09 (m, 1H), |
| | | 1.97-1.87 (m, 2H), 1.75-1.63 |
| | | (m, 2H), 1.56-1.45 (m, 4H). |
| 333 | 561.6, | 1H NMR (400 MHz, DMSO): δ 9.99 (s, 1H), |
| | 563.9 | 9.15 (s, 1H), 9.09 (d, J = 1.9 Hz, |
| | | 1H), 9.04 (s, 1H), 8.11 (d, J = 5.7 Hz, |
| | | 1H), 7.75 (dd, J = 7.4, 1.2 Hz, 1H), 7.72 |
| | | (s, 1H), 7.54-7.47 (m, 1H), 7.32 (s, 1H), 7.23 |
| | | (d, J = 8.6 Hz, 1H), 6.90 (d, J = |
| | | 8.7 Hz, 1H), 6.33 (d, J = 5.7 Hz, 1H), |
| | | 4.37-4.31 (m, 1H), 3.65 (s, 3H), 3.62- |
| | | 3.56 (m, 4H), 2.49-2.44 (m, 4H), |
| | | 2.26-2.16 (m, 1H), 1.98-1.87 (m, 2H), |
| | | 1.78-1.66 (m, 2H), 1.60-1.47 (m, 4H). |
| 334 | 525.3 | 1H NMR (400 MHz, DMSO) δ 10.02 (s, 1H), |
| | | 9.22 (s, 1H), 9.11-9.06 (m, |
| | | 2H), 8.22 (s, 1H), 8.15-8.10 (m, 2H), |
| | | 7.83-7.80 (m, 1H), 7.32 (s, 1H), 7.22 |
| | | (d, J = 8.3 Hz, 1H), 6.67 (d, J = 8.7 Hz, |
| | | 1H), 6.33 (d, J = 5.7 Hz, 1H), 4.67- |
| | | 4.61 (m, 1H), 3.67 (s, 3H), 2.83-2.79 |
| | | (m, 1H), 2.30-2.24 (m, 2H), 2.16- |
| | | 2.09 (m, 2H), 2.06 (s, 6H). |
| 335 | 471.4 | 1H NMR (400 MHz, DMSO) δ 9.76 (s, 1H), |
| | | 9.04 (s, 1H), 8.83 (t, J = 5.3 Hz, |
| | | 2H), 8.07 (d, J = 5.7 Hz, 1H), 7.81 (d, |
| | | J = 8.5 Hz, 1H), 7.48 (s, 1H), 7.44- |
| | | 7.40 (m, 1H), 7.27 (s, 1H), 7.19 (d, J = 8.8 |
| | | Hz, 1H), 6.67 (d, J = 8.7 Hz, 1H), |
| | | 6.28 (d, J = 5.7 Hz, 1H), 4.70-4.65 |
| | | (m, 1H), 3.64 (s, 3H), 2.84-2.79 (m, |
| | | 1H), 2.51 (s, 3H), 2.31-2.26 (m, 2H), |
| | | 2.17-2.11 (m, 2H), 2.07 (s, 6H). |
| 336 | 471.6 | 1H NMR (500 MHz, DMSO) δ 9.74 |
| | | (s, 1H), 9.04 (s, 1H), 8.90-8.87 (m, 2H), |
| | | 8.06 (d, J = 5.7 Hz, 1H), 7.72 (s, 1H), 7.64 |
| | | (s, 1H), 7.39 (d, J = 8.2 Hz, 1H), |
| | | 7.26 (s, 1H), 7.21 (s, 1H), 6.65 (s, 1H), |
| | | 6.27 (d, J = 5.7 Hz, 1H), 4.68-4.65 |
| | | (m, 1H), 3.63 (s, 3H), 2.84-2.81 (m, 1H), |
| | | 2.51 (s, 3H), 2.31-2.27 (m, 2H), |
| | | 2.17-2.13 (m, 2H), 2.08 (s, 6H). |
| 337 | 487.4 | 1H NMR (500 MHz, DMSO) δ 9.66 (s, 1H), |
| | | 9.01 (s, 1H), 8.88-8.86 (m, 1H), |
| | | 8.84 (s, 1H), 8.04 (d, J = 5.7 Hz, 1H), |
| | | 7.66 (s, 1H), 7.33 (d, J = 2.5 Hz, 1H), |
| | | 7.27 (s, 1H), 7.23-7.19 (m, 2H), 6.64 (d, |
| | | J = 8.7 Hz, 1H), 6.25 (d, J = 5.7 Hz, |
| | | 1H), 4.67-4.64 (m, 1H), 3.90 (s, 3H), |
| | | 3.63 (s, 3H), 2.83-2.80 (m, 1H), 2.30- |
| | | 2.26 (m, 2H), 2.16-2.12 (m, 2H), 2.07 (s, 6H). |
| 338 | 489.5 | 1H NMR (400 MHz, DMSO) δ 9.33 (s, |
| | | 1H), 8.93 (s, 1H), 8.43 (s, 1H), 7.98 (d, |
| | | J = 5.7 Hz, 1H), 7.95 (d, J = 2.1 Hz, 1H), |
| | | 7.22 (s, 1H), 7.17 (dd, J = 8.8, 2.1 |
| | | Hz, 1H), 6.62 (d, J = 8.8 Hz, 1H), 6.15 |
| | | (d, J = 5.9 Hz, 1H), 4.70-4.50 (m, |
| | | 1H), 3.63 (s, 3H), 2.80 (tt, J = 7.6, 5.1 Hz, |
| | | 1H), 2.69 (t, J = 6.0 Hz, 2H), 2.26 |
| | | (m, 2H), 2.17-2.07 (m, 2H), 2.07 (s, 6H), |
| | | 1.99 (s, 2H) 1.53 (t, J = 6.7 Hz, |
| | | 2H), 0.98 (s, 6H). |
| 339 | 489.4 | 1H NMR (400 MHz, CD3CN) δ 8.42 (d, |
| | | J = 2.0 Hz, 1H), 7.98 (d, J = 5.7 Hz, |
| | | 1H), 7.79 (d, J = 2.1 Hz, 1H), 7.58 (s, 1H), |
| | | 7.47 (s, 1H), 7.24 (d, J = 2.1 Hz, |
| | | 1H), 7.05 (dd, J = 8.6, 2.3 Hz, 1H), 6.65 |
| | | (d, J = 8.6 Hz, 1H), 6.12 (d, J = 5.9 |
| | | Hz, 1H), 4.69 (tt, J = 7.2, 3.7 Hz, 1H), |
| | | 3.70 (s, 3H), 2.84 (m, J = 13.3, 4.4 Hz, |
| | | 3H), 2.48 (s, 2H), 2.38-2.28 (m, 2H), 2.19 |
| | | (s, 8H), 1.66 (t, J = 6.9 Hz, 2H), |
| | | 1.00 (s, 6H). |
| 340 | 487.4 | 1H NMR (500 MHz, MeOD) δ 8.91 (s, 1H), 7.88 |
| | | (d, J = 5.8 Hz, 1H), 7.63 (d, J = |
| | | 7.9 Hz, 1H), 7.38 (m, 2H), 7.27-7.20 |
| | | (m, 1H), 7.10 (d, J = 2.4 Hz, 1H), |
| | | 6.98 (dd, J = 8.5, 2.4 Hz, 1H), 6.61 (d, |
| | | J = 8.6 Hz, 1H), 6.33 (d, J = 5.9 Hz, |
| | | 1H), 4.06 (s, 3H), 3.58 (s, 3H), 3.19-3.14 |
| | | (m, 1H), 2.37-2.31 (m, 4H), 2.25 |
| | | (m, 7H). |
| 341 | 525.3 | 1H NMR (400 MHz, DMSO) δ 10.06 (s, 1H), |
| | | 9.16 (s, 2H), 9.06 (d, J = 2.5 Hz, |
| | | 1H), 8.25 (s, 1H), 8.14 (d, J = 5.7 Hz, 1H), |
| | | 7.94 (s, 1H), 7.81 (d, J = 8.6 Hz, |
| | | 1H), 7.30-7.18 (m, 2H), 6.72 (d, J = 8.6 Hz, |
| | | 1H), 6.35 (d, J = 5.7 Hz, 1H), |
| | | 4.73-4.66 (m, 1H), 3.68 (s, 3H), |
| | | 2.87-2.79 (m, 1H), 2.36-2.26 (m, 2H), |
| | | 2.20-2.13 (m, 2H), 2.08 (s, 6H). |
| 342 | 491.6, | 1H NMR (500 MHz, DMSO): δ 9.89 (s, |
| | 493.4 | 1H), 9.08 (s, 1H), 9.01 (s, 1H), 8.97 |
| | | (d, J = 2.3 Hz, 1H), 8.09 (d, J = 5.7 Hz, |
| | | 1H), 7.97 (d, J = 1.9 Hz, 1H), 7.78 (s, |
| | | 1H), 7.58 (dd, J = 8.7, 2.2 Hz, 1H), 7.26 |
| | | (s, 1H), 7.21 (d, J = 8.4 Hz, 1H), 6.75 |
| | | (d, J = 8.7 Hz, 1H), 6.30 (d, J = 5.7 Hz, |
| | | 1H), 4.34 (p, J = 7.2 Hz, 1H), 3.64 (s, |
| | | 3H), 2.60-2.54 (m, 2H), 2.35-2.26 |
| | | (m, 1H), 2.06 (s, 6H), 1.88-1.79 (m, |
| | | 2H). |
| 343 | 491.4, | 1H NMR (500 MHz, DMSO): δ 9.92 (s, |
| | 493.6 | 1H), 9.07 (s, 1H), 9.02 (s, 1H), 8.92 |
| | | (d, J = 2.4 Hz, 1H), 8.10 (d, J = 5.6 Hz, 1H), |
| | | 7.94 (d, J = 8.9 Hz, 1H), 7.82 (s, |
| | | 1H), 7.58 (dd, J = 8.9, 2.4 Hz, 1H), |
| | | 7.28-7.20 (m, 2H), 6.77 (d, J = 9.0 Hz, |
| | | 1H), 6.31 (d, J = 5.7 Hz, 1H), 4.40-4.31 |
| | | (m, 1H), 3.66 (s, 3H), 2.61-2.53 |
| | | (m, 2H), 2.35-2.27 (m, 1H), 2.05 |
| | | (s, 6H), 1.88-1.79 (m, 2H). |
| 344 | 491.8, | 1H NMR (500 MHz, DMSO): δ 9.97 (s, 1H), |
| | 493.6 | 9.11 (s, 1H), 9.08 (s, 1H), 9.01 |
| | | (s, 1H), 8.10 (dd, J = 5.6, 1.0 Hz, 1H), |
| | | 7.76 (d, J = 7.4 Hz, 1H), 7.72 (s, 1H), |
| | | 7.51 (t, J = 7.8 Hz, 1H), 7.27 (s, 1H), |
| | | 7.23 (d, J = 8.2 Hz, 1H), 6.77 (d, J = 8.7 |
| | | Hz, 1H), 6.32 (d, J = 5.7 Hz, 1H), 4.34 (p, |
| | | J = 7.1 Hz, 1H), 3.65 (s, 3H), 2.61- |
| | | 2.54 (m, 2H), 2.35-2.28 (m, 1H), 2.05 |
| | | (s, 6H), 1.87-1.79 (m, 2H). |
| 345 | 499.4 | 1H NMR (500 MHz, DMSO): δ 9.65 (s, 1H), |
| | | 9.03 (s, 1H), 8.85 (d, J = 2.5 Hz, |
| | | 1H), 8.82 (s, 1H), 8.03 (d, J = 5.7 Hz, 1H), |
| | | 7.70 (d, J = 9.0 Hz, 1H), 7.55 (d, J = |
| | | 8.9 Hz, 2H), 7.33 (d, J = 2.6 Hz, 1H), |
| | | 7.23 (dd, J = 8.9, 2.6 Hz, 1H), 6.87- |
| | | 6.80 (m, 2H), 6.24 (d, J = 5.7 Hz, 1H), |
| | | 5.21-5.09 (m, 1H), 3.90 (s, 3H), 3.78- |
| | | 3.64 (m, 4H), 2.75 (s, 2H), 2.44 (s, 3H), |
| | | 1.94 (dd, J = 8.2, 3.0 Hz, 4H). |
| 346 | 499.4 | 1H NMR (500 MHz, DMSO): δ 9.65 (s, 1H), |
| | | 9.01 (s, 1H), 8.84 (s, 2H), 8.03 |
| | | (d, J = 5.7 Hz, 1H), 7.69 (d, J = 8.9 Hz, 1H), |
| | | 7.53 (d, J = 8.7 Hz, 2H), 7.33 (d, J = |
| | | 2.5 Hz, 1H), 7.21 (dd, J = 8.9, 2.6 Hz, 1H), |
| | | 6.85-6.78 (m, 2H), 6.24 (d, J = |
| | | 5.7 Hz, 1H), 4.62 (p, J = 6.2 Hz, 1H), 3.90 |
| | | (s, 3H), 3.79 (dd, J = 10.9, 2.3 Hz, |
| | | 2H), 3.38-3.36 (m, 2H), 2.67 (d, J = 8.6 |
| | | Hz, 2H), 2.43 (s, 3H), 2.43-2.34 |
| | | (m, 2H), 1.61 (dd, J = 14.4, 3.1 Hz, 2H). |
| 347 | 595.5, | 1H NMR (400 MHz, DMSO) δ 9.91 (s, 1H), |
| | 596.8 | 9.07 (s, 1H), 8.98 (s, 1H), 8.90 (s, |
| | | 1H), 8.08 (d, J = 5.6 Hz, 1H), 7.96 (s, 1H), |
| | | 7.88-7.83 (m, 1H), 7.69 (d, J = 8.9 |
| | | Hz, 1H), 7.59-7.52 (m, 2H), 6.78 (d, |
| | | J = 8.5 Hz, 2H), 6.29 (d, J = 4.9 Hz, |
| | | 1H), 3.25-3.22 (m, 1H), 3.10 (s, 4H), |
| | | 2.78 (s, 4H), 2.38-2.32 (m, 2H), 2.21 |
| | | (s, 2H). |
| 348 | 525.5 | 1H NMR (400 MHz, DMSO) δ 9.26 (s, 1H), |
| | | 8.98 (s, 1H), 7.95 (d, J = 5.7 Hz, |
| | | 2H), 7.79-7.77 (m, 1H), 7.55 (d, |
| | | J = 9.0 Hz, 2H), 6.76-6.69 (m, 2H), 6.10 |
| | | (d, J = 5.7 Hz, 1H), 4.69-4.64 (m, 1H), |
| | | 4.39-4.35 (m, 2H), 4.28-4.23 (m, |
| | | 2H), 3.25-3.20 (m, 1H), 3.13-3.06 |
| | | (m, 4H), 2.79 (s, 4H), 2.36-2.30 (m, |
| | | 2H), 2.21-2.14 (m, 2H). |
| 349 | 551.7, | 1H NMR (500 MHz, DMSO) δ 9.95 (s, 1H), |
| | 553.8 | 9.12 (s, 1H), 9.06 (s, 1H), 8.98 (s, |
| | | 1H), 8.09 (d, J = 5.7 Hz, 1H), |
| | | 7.78-7.71 (m, 2H), 7.58-7.50 (m, 3H), 6.82- |
| | | 6.76 (m, 2H), 6.31 (d, J = 5.7 Hz, 1H), |
| | | 4.71-4.68 (m, 1H), 3.26-3.24 (m, |
| | | 1H), 3.13-3.09 (m, 4H), 2.79 (s, 4H), |
| | | 2.38-2.32 (m, 2H), 2.24-2.18 (m, |
| | | 2H). |
| 350 | 550.4, | 1H NMR (500 MHz, DMSO) δ 9.88 (s, 1H), |
| | 551.7 | 9.09 (s, 1H), 8.95 (s, 2H), 8.08 (d, |
| | | J = 5.7 Hz, 1H), 7.98 (d, J = 2.1 Hz, |
| | | 1H), 7.79 (s, 1H), 7.61-7.54 (m, 3H), |
| | | 6.80-6.74 (m , 2H), 6.29 (d, J = 5.7 Hz, |
| | | 1H), 4.72-4.68 (m, 1H), 3.26-3.24 |
| | | (m, 1H), 3.14-3.08 (m, 4H), 2.80 (s, 4H), |
| | | 2.37-2.32 (m, 2H), 2.23-2.17 |
| | | (m, 2H). |
| 351 | 533.4 | 1HNMR(500 MHz, DMSO) δ 9.27 (s, 1H), |
| | | 8.94 (s, 1H), 8.21 (s, 1H), 8.03 (d, J = |
| | | 2.3 Hz, 1H), 7.96 (d, J = 5.7 Hz, 1H), |
| | | 7.58-7.51 (m, 2H), 6.77-6.70 (m, |
| | | 2H), 6.13 (d, J = 5.7 Hz, 1H), 4.70-4.65 |
| | | (m, 1H), 3.37 (s, 1H), 3.29-3.22 |
| | | (m, 2H), 3.11 (m, 4H), 2.79 (s, 4H), 2.38-2.32 |
| | | (m, 2H), 2.20 (d, J = 8.0 Hz, |
| | | 2H), 1.96 (d, J = 8.6 Hz, 1H), 1.88-1.79 |
| | | (m, 1H), 1.74 (d, J = 9.0 Hz, 1H), |
| | | 1.57 (d, J = 8.9 Hz, 1H), 1.33-1.23 (m, |
| | | 1H), 1.14 (d, J = 9.4 Hz, 1H). |
| 352 | 499.4 | ¹H NMR (500 MHz, DMSO): δ 9.65 (s, 1H), |
| | | 9.02 (s, 1H), 8.84 (d, J = 2.4 Hz, |
| | | 1H), 8.80 (s, 1H), 8.03 (d, J = 5.7 Hz, 1H), |
| | | 7.68 (d, J = 8.8 Hz, 1H), 7.53 (d, J = |
| | | 8.8 Hz, 2H), 7.34 (d, J = 2.5 Hz, 1H), |
| | | 7.23 (dd, J = 8.9, 2.6 Hz, 1H), 6.76 (d, |
| | | J = 9.0 Hz, 2H), 6.24 (d, J = 5.7 Hz, 1H), |
| | | 4.42 (p, J = 7.3 Hz, 1H), 3.90 (s, 3H), |
| | | 3.58 (t, J = 4.6 Hz, 4H), 2.66-2.57 |
| | | (m, 2H), 2.48-2.40 (m, 1H), 2.31-2.21 |
| | | (m, 4H), 1.90-1.77 (m, 2H). |
| 353 | 547.4 | ¹H NMR (400 MHz, DMSO): δ 9.61 (s, 1H), |
| | | 8.97 (s, 1H), 8.78 (s, 1H), 8.76 (s, |
| | | 1H), 8.18 (s, 1H), 7.98 (d, J = 5.7 Hz, 1H), |
| | | 7.64 (d, J = 9.3 Hz, 1H), 7.50 (d, J = |
| | | 8.7 Hz, 2H), 7.29 (d, J = 2.4 Hz, 1H), |
| | | 7.18 (dd, J = 8.9, 2.5 Hz, 1H), 6.68 (d, |
| | | J = 9.0 Hz, 2H), 6.19 (d, J = 5.7 Hz, |
| | | 1H), 4.68-4.58 (m, 1H), 3.85 (s, 3H), |
| | | 3.18-3.16 (m, 1H), 3.08-3.04 (m, 4H), |
| | | 2.78-2.71 (m, 4H), 2.34-2.25 (m, |
| | | 2H), 2.19-2.08 (m, 2H). |
| 354 | 497.4 | ¹H NMR (500 MHz, DMSO): δ 9.68 (s, 1H), |
| | | 9.05 (s, 1H), 8.83 (d, J = 9.8 Hz, |
| | | 2H), 8.04 (d, J = 5.7 Hz, 1H), 7.69 (d, |
| | | J = 5.3 Hz, 1H), 7.55 (d, J = 8.8 Hz, |
| | | 2H), 7.34 (d, J = 2.4 Hz, 1H), 7.23 (dd, |
| | | J = 8.9, 2.5 Hz, 1H), 6.72 (d, J = 9.0 |
| | | Hz, 2H), 6.24 (d, J = 5.7 Hz, 1H), |
| | | 4.71-4.62 (m, 1H), 3.91 (s, 3H), 2.92- |
| | | 2.82 (m, 1H), 2.57-2.52 (m, 2H), 2.35-2.29 |
| | | (m, 2H), 2.26-2.21 (m, 2H), |
| | | 2.16-2.10 (m, 2H), 1.55-1.47 |
| | | (m, 4H), 1.46-1.31 (m, 2H). |
| 355 | 457.5 | ¹H NMR (500 MHz, DMSO): δ 9.67 (s, 1H), |
| | | 9.05 (s, 1H), 8.83 (d, J = 9.6 Hz, |
| | | 2H), 8.03 (d, J = 5.8 Hz, 1H), 7.69 (d, |
| | | J = 10.5 Hz, 1H), 7.54 (d, J = 8.6 Hz, |
| | | 2H), 7.33 (d, J = 2.5 Hz, 1H), 7.22 (dd, |
| | | J = 8.9, 2.5 Hz, 1H), 6.72 (d, J = 9.0 |
| | | Hz, 2H), 6.24 (d, J = 5.7 Hz, 1H), |
| | | 4.73-4.60 (m, 1H), 3.90 (s, 3H), 2.82 (t, J = |
| | | 5.5 Hz, 1H), 2.39-2.27 (m, 2H), |
| | | 2.17-2.09 (m, 2H), 2.08 (s, 6H). |
| 356 | 499.4 | ¹H NMR (400 MHz, DMSO): δ 9.65 (s, 1H), |
| | | 9.02 (s, 1H), 8.84 (d, J = 2.5 Hz, |
| | | 1H), 8.81 (s, 1H), 8.03 (d, J = 5.7 Hz, 1H), |
| | | 7.69 (d, J = 8.9 Hz, 1H), 7.54 (d, J = |
| | | 8.9 Hz, 2H), 7.34 (d, J = 2.5 Hz, 1H), |
| | | 7.22 (dd, J = 8.9, 2.6 Hz, 1H), 6.78- |
| | | 6.67 (m, 2H), 6.24 (d, J = 5.7 Hz, 1H), |
| | | 4.73-4.64 (m, 1H), 3.90 (s, 3H), 3.61 |
| | | (t, J = 4.5 Hz, 4H), 2.95-2.87 (m, 1H), |
| | | 2.40-2.28 (m, 6H), 2.16-2.09 (m, |
| | | 2H). |
| 357 | 465.3 | 1HNMR(500 MHz, DMSO) δ 9.27 (s, 1H), |
| | | 8.95 (s, 1H), 7.99-7.96 (m, 2H), |
| | | 7.78 (d, J = 2.3 Hz, 1H), 7.29 (s, 1H), |
| | | 7.22-7.19 (m, 1H), 6.62 (d, J = 8.8 Hz, |
| | | 1H), 6.12 (d, J = 5.7 Hz, 1H), 4.64-4.59 |
| | | (m, 1H), 4.39-4.35 (m, 2H), 4.26- |
| | | 4.23 (m, 2H), 3.67 (s, 3H), 2.83-2.78 (m, |
| | | 1H), 2.32-2.23 (m, 2H), 2.15- |
| | | 2.10 (m, 2H), 2.10-2.05 (m, 6H). |
| 358 | 491.7, | 1HNMR(500 MHz, DMSO) δ 9.97 (s, 1H), |
| | 493.5 | 9.11 (s, 1H), 9.07 (s, 1H), 9.02 (s, |
| | | 1H), 8.10 (d, J = 5.7 Hz, 1H), 7.77-7.75 |
| | | (m, 1H), 7.51 (d, J = 7.8 Hz, 1H), |
| | | 7.27 (s, 1H), 7.24-7.21 (m, 1H), 6.67 (s, 1H), |
| | | 6.31 (s, 1H), 4.67 (s, 1H), 3.66 |
| | | (s, 3H), 2.82 (s, 2H), 2.31-2.27 (m, 2H), |
| | | 2.17-2.13 (m, 2H), 2.06 (s, 6H). |
| 359 | 555.3 | 1H NMR (500 MHz, DMSO): δ 9.28 (s, 1H), |
| | | 8.96 (s, 1H), 8.00 (d, J = 2.0 Hz, |
| | | 1H), 7.98 (d, J = 5.7 Hz, 1H), 7.77 (d, |
| | | J = 2.2 Hz, 1H), 7.32 (d, J = 2.2 Hz, |
| | | 1H), 7.22 (dd, J = 8.7, 2.4 Hz, 1H), |
| | | 6.64 (d, J = 8.8 Hz, 1H), 6.12 (d, J = 5.7 |
| | | Hz, 1H), 4.67-4.59 (m, 1H), 4.40-4.35 |
| | | (m, 2H), 4.27-4.23 (m, 2H), 3.68 |
| | | (s, 3H), 3.27-3.19 (m, 1H), 3.14-3.07 |
| | | (m, 4H), 2.82-2.75 (m, 4H), 2.34- |
| | | 2.26 (m, 2H), 2.23-2.15 (m, 2H). |
| 360 | 505.4 | 1H NMR (500 MHz, DMSO): δ 9.19 (s, 1H), |
| | | 8.90 (s, 1H), 8.13 (s, 1H), 7.95 |
| | | (d, J = 5.7 Hz, 1H), 7.87 (s, 1H), 7.26 (s, |
| | | 1H), 7.15 (d, J = 8.6 Hz, 1H), 6.62 (d, |
| | | J = 8.7 Hz, 1H), 6.10 (d, J = 5.7 Hz, |
| | | 1H), 4.66-4.60 (m, 1H), 4.26-4.21 (m, |
| | | 2H), 3.63 (s, 3H), 3.59 (t, J = 4.6 Hz, 4H), |
| | | 2.92-2.83 (m, 1H), 2.71 (t, J = 6.4 |
| | | Hz, 2H), 2.34-2.23 (m, 6H), 2.15-2.06 |
| | | (m, 2H), 1.94-1.86 (m, 2H). |
| 361 | 533.4, | 1H NMR (500 MHz, DMSO): δ 9.93 (s, 1H), |
| | 535.3 | 9.07 (s, 1H), 9.01 (s, 1H), 8.91 |
| | | (d, J = 2.4 Hz, 1H), 8.10 (d, J = 5.7 Hz, |
| | | 1H), 7.93 (d, J = 8.9 Hz, 1H), 7.81 (s, |
| | | 1H), 7.58 (dd, J = 8.9, 2.4 Hz, 1H), 7.27 |
| | | (s, 1H), 7.21 (d, J = 8.5 Hz, 1H), 6.69 |
| | | (d, J = 8.7 Hz, 1H), 6.31 (d, J = 5.7 Hz, |
| | | 1H), 4.74-4.67 (m, 1H), 3.67 (s, 3H), |
| | | 3.59 (t, J = 4.5 Hz, 4H), 2.96-2.85 |
| | | (m, 1H), 2.39-2.22 (m, 6H), 2.19-2.10 |
| | | (m, 2H). |
| 362 | 384.3 | 1H NMR (400 MHz, DMSO): δ 9.89 (s, 1H), |
| | | 9.28 (s, 1H), 8.99-8.93 (m, |
| | | 2H), 8.13 (d, J = 5.7 Hz, 1H), 7.94 (d, |
| | | J = 8.6 Hz, 1H), 7.85 (d, J = 7.3 Hz, |
| | | 1H), 7.63-7.53 (m, 2H), 7.36 (s, 1H), 7.27 |
| | | (d, J = 8.1 Hz, 1H), 7.13 (t, J = 8.1 |
| | | Hz, 1H), 6.43 (dd, J = 8.1, 1.6 Hz, 1H), |
| | | 6.35 (d, J = 5.7 Hz, 1H), 4.55 (p, J = |
| | | 7.5 Hz, 1H), 2.36-2.30 (m, 2H), 2.00-1.96 |
| | | (m, 2H), 1.72 (q, J = 9.7 Hz, |
| | | 1H), 1.63-1.49 (m, 1H). |
| 363 | 467.7 | ¹H NMR (500 MHz, DMSO): δ 9.80 (s, 1H), |
| | | 9.07 (s, 1H), 8.92 (s, 1H), 8.90 (s, |
| | | 1H), 8.06 (d, J = 5.7 Hz, 1H), 7.93 (d, |
| | | J = 8.3 Hz, 1H), 7.74 (s, 1H), 7.62- |
| | | 7.50 (m, 4H), 6.83-6.75 (m, 2H), 6.28 (d, |
| | | J = 5.7 Hz, 1H), 4.41 (p, J = 7.2 Hz, |
| | | 1H), 2.69-2.59 (m, 2H), 2.41-2.32 (m, |
| | | 1H), 2.22 (brs, 4H), 1.86-1.77 (m, |
| | | 2H), 1.56-1.45 (m, 4H), 1.43-1.33 (m, 2H). |
| 364 | 461.6 | 1H NMR (400 MHz, CD3OD): δ 8.32 (d, |
| | | J = 2.4 Hz, 1H), 8.02 (d, J = 2.5 Hz, |
| | | 1H), 7.92 (d, J = 5.9 Hz, 1H), 7.18 (d, |
| | | J = 2.4 Hz, 1H), 6.99 (dd, J = 8.6, 2.4 |
| | | Hz, 1H), 6.80 (d, J = 8.6 Hz, 1H), |
| | | 6.15 (d, J = 5.9 Hz, 1H), 4.44-4.34 (m, |
| | | 1H), 3.67 (s, 3H), 2.83 (t, J = 6.4 Hz, |
| | | 2H), 2.72-2.61 (m, 4H), 2.54-2.40 (m, |
| | | 1H), 2.19 (s, 6H), 2.08-1.96 (m, 2H), |
| | | 1.95-1.86 (m, 2H), 1.85-1.76 (m, |
| | | 2H). |
| 365 | 463.5 | 1H NMR (400 MHz, DMSO): δ 9.19 (s, 1H), |
| | | 8.89 (s, 1H), 8.14 (s, 1H), 7.95 |
| | | (d, J = 5.7 Hz, 1H), 7.87 (d, J = 2.7 Hz, 1H), |
| | | 7.26 (d, J = 2.1 Hz, 1H), 7.15 (dd, |
| | | J = 8.4, 2.1 Hz, 1H), 6.71 (d, J = 8.7 Hz, |
| | | 1H), 6.10 (d, J = 5.8 Hz, 1H), 4.35- |
| | | 4.26 (m, 1H), 4.26-4.20 (m, 2H), 3.62 |
| | | (s, 3H), 2.76-2.68 (m, 2H), 2.59- |
| | | 2.52 (m, 2H), 2.34-2.24 (m, 1H), 2.04 |
| | | (s, 6H), 1.94-1.86 (m, 2H), 1.85- |
| | | 1.74 (m, 2H). |
| 366 | 479.6 | 1H NMR (400 MHz, DMSO): δ 9.36 (s, 1H), |
| | | 9.00 (s, 1H), 8.38 (s, 1H), 8.00 |
| | | (d, J = 5.7 Hz, 1H), 7.92 (d, J = 2.4 Hz, |
| | | 1H), 7.26-7.15 (m, 2H), 6.76 (d, J = |
| | | 8.5 Hz, 1H), 6.16 (d, J = 5.7 Hz, 1H), |
| | | 4.36-4.25 (m, 1H), 3.66 (s, 3H), 3.08- |
| | | 2.98 (m, 2H), 2.89-2.75 (m, 2H), |
| | | 2.60-2.52 (m, 2H), 2.34-2.25 (m, 1H), |
| | | 2.17-2.08 (m, 2H), 2.04 (s, 6H), 1.81 (ddd, |
| | | J = 16.2, 8.5, 2.8 Hz, 2H). |
| 367 | 427.5 | ¹H NMR (500 MHz, DMSO): δ 9.80 (s, 1H), |
| | | 9.07 (s, 1H), 8.92 (s, 1H), 8.90 |
| | | (d, J = 2.2 Hz, 1H), 8.06 (d, J = 5.7 Hz, 1H), |
| | | 7.93 (d, J = 8.3 Hz, 1H), 7.75 (d, J = |
| | | 6.2 Hz, 1H), 7.59 (ddd, J = 8.4, 6.9, |
| | | 1.6 Hz, 1H), 7.57-7.50 (m, 3H), 6.82- |
| | | 6.77 (m, 2H), 6.28 (d, J = 5.7 Hz, 1H), |
| | | 4.38 (p, J = 7.2 Hz, 1H), 2.64-2.59 (m, |
| | | 2H), 2.38-2.32 (m, 1H), 2.06 (s, 6H), |
| | | 1.87-1.77 (m, 2H). |
| 368 | 412.3 | 1H NMR (400 MHz, DMSO): δ 9.89 (s, 1H), |
| | | 9.25 (s, 1H), 8.97 (d, J = 17.8 |
| | | Hz, 2H), 8.12 (d, J = 5.6 Hz, 1H), 7.93 |
| | | (d, J = 7.9 Hz, 1H), 7.84 (d, J = 7.9 Hz, |
| | | 1H), 7.65-7.52 (m, 2H), 7.39 (s, 1H), 7.28 |
| | | (d, J = 8.2 Hz, 1H), 7.14 (t, J = 8.1 |
| | | Hz, 1H), 6.54 (d, J = 7.5 Hz, 1H), 6.35 |
| | | (d, J = 5.6 Hz, 1H), 4.18 (t, J = 8.6 Hz, |
| | | 1H), 1.90-1.81 (m, 2H), 1.70-1.60 (m, 2H), |
| | | 1.52-1.44 (m, 1H), 1.38-1.20 |
| | | (m, 5H). |
| 369 | 457.5 | 1H NMR (500 MHz, DMSO): δ 9.82 (s, 1H), |
| | | 9.08 (s, 1H), 8.96 (s, 1H), 8.92 |
| | | (d, J = 2.3 Hz, 1H), 8.08 (d, J = 5.7 Hz, |
| | | 1H), 7.92 (d, J = 8.4 Hz, 1H), 7.74 (s, |
| | | 1H), 7.59 (ddd, J = 8.4, 6.9, 1.6 Hz, 1H), |
| | | 7.54 (t, J = 6.9 Hz, 1H), 7.27 (s, 1H), |
| | | 7.23 (d, J = 7.9 Hz, 1H), 6.67 (d, J = 8.7 Hz, |
| | | 1H), 6.29 (d, J = 5.7 Hz, 1H), 4.70- |
| | | 4.62 (m, 1H), 3.64 (s, 3H), 2.91-2.82 |
| | | (m, 1H), 2.35-2.24 (m, 2H), 2.22- |
| | | 2.12 (m, 2H), 2.10 (s, 6H). |
| 370 | 543.8 | 1H NMR (400 MHz, DMSO) δ 9.31 (s, 1H), |
| | | 8.95 (s, 1H), 8.63 (s, 1H), 7.99 (d, |
| | | J = 5.7 Hz, 1H), 7.73 (d, J = 2.3 Hz, 1H), |
| | | 7.26 (d, J = 1.2 Hz, 1H), 7.18 (dd, J = |
| | | 8.7, 2.0 Hz, 1H), 6.81 (d, J = 8.8 Hz, 1H), |
| | | 6.16 (d, J = 5.7 Hz, 1H), 3.92 (t, J = |
| | | 6.4 Hz, 2H), 3.63 (s, 3H), 2.66 (t, J = 4.6 |
| | | Hz, 2H), 2.39-2.28 (m, 6H), 2.11- |
| | | 1.99 (m, 2H), 1.87-1.78 (m, 4H), |
| | | 1.74-1.66 (m, 6H), 1.62-1.54 (m, 2H), |
| | | 1.52-1.46 (m, 4H), 1.42-1.33 (m, 2H). |
| 371 | 521.4 | 1HNMR(500 MHz, DMSO) δ 8.20 (s, 1H), |
| | | 8.01 (s, 1H), 7.95-7.93 (m, 1H), |
| | | 7.60-7.46 (m, 4H), 6.90-6.85 (m, 3H), |
| | | 6.17 (m, 1H), 3.99-3.97 (m, 2H), |
| | | 3.09 (s, 5H), 2.92 (s, 5H), 2.63 (s, 2H), |
| | | 2.08 (s, 1H), 1.97-1.85 (m, 5H), 1.75- |
| | | 1.71 (m, 1H), 1.58-1.55 (m, 1H), 1.32-1.23 |
| | | (m, 4H), 1.14-1.11 (m, 1H). |
| 372 | 473.4 | 1H NMR (500 MHz, DMSO) δ 9.29 (s, 1H), |
| | | 8.95 (s, 1H), 8.19 (s, 1H), 8.05 (s, |
| | | 1H), 7.95 (s, 1H), 7.56-7.50 (m, 1H), |
| | | 6.88-6.82 (m, 1H), 6.13-6.11 (m, |
| | | 1H), 3.98-3.95 (m,2H), 3.58-3.56 (m, 3H), |
| | | 3.35 (s, 2H), 3.25 (s, 1H), 2.54- |
| | | 2.52 (m, 1H), 2.44-2.40 (m, 2H), 2.36 |
| | | (s, 3H), 2.00-1.93 (m, 2H), 1.88- |
| | | 1.84 (m, 2H), 1.75-1.70 (m, 1H), 1.57-1.53 |
| | | (m, 1H), 1.16-1.11 (m, 2H). |
| 373 | 487.5 | 1H NMR (500 MHz, DMSO) δ 9.35 (s, 1H), |
| | | 8.96 (s, 1H), 8.31 (s, 1H), 8.14- |
| | | 8.12 (m, 1H), 7.98-7.96 (m, 1H), 7.53-7.48 |
| | | (m, 2H), 6.82 (d, 2H), 6.15 (d, J = |
| | | 5.7 Hz, 1H), 3.95 (t, J = 6.4 Hz, 2H), |
| | | 3.60-3.56 (m, 4H), 3.02-2.97 (m, |
| | | 2H), 2.43-2.40 (m, 2H), 2.36 (s, 4H), |
| | | 1.89-1.84 (m, 2H), 1.82-1.75 (m, |
| | | 4H), 1.38-1.29 (m, 4H). |
| 374 | 489.5 | 1H NMR (500 MHz, DMSO) δ 9.33 (s, 1H), |
| | | 8.98 (s, 1H), 8.49 (s, 1H), 8.00 (d, |
| | | J = 5.7 Hz, 1H), 7.87 (d, J = 2.0 Hz, 1H), |
| | | 7.31 (s, 1H), 7.19 (d, J = 8.0 Hz, 1H), |
| | | 6.86 (d, J = 8.7 Hz, 1H), 6.17 (d, J = 5.7 |
| | | Hz, 1H), 3.97 (t, J = 6.1 Hz, 2H), 3.64 |
| | | (s, 3H), 2.86 (broad, 6H), 2.76 (t, J = 6.4 Hz, |
| | | 2H), 2.67 (t, J = 6.0 Hz, 2H), 2.00 |
| | | (s, 2H), 1.87-1.78 (m, 2H), 1.77-1.70 (m, |
| | | 2H), 1.64 (s, 4H), 1.49 (s, 2H). |
| 375 | 491.5 | 1H NMR (500 MHz, DMSO) δ 9.33 (s, 1H), |
| | | 8.98 (s, 1H), 8.49 (s, 1H), 8.00 (d, |
| | | J = 5.7 Hz, 1H), 7.87 (d, J = 2.0 Hz, |
| | | 1H), 7.31 (s, 1H), 7.19 (d, J = 8.0 Hz, 1H), |
| | | 6.86 (d, J = 8.7 Hz, 1H), 6.17 (d, J = 5.7 |
| | | Hz, 1H), 3.97 (t, J = 6.1 Hz, 2H), 3.64 |
| | | (s, 3H), 2.86 (broad s, 4H), 2.76 (t, J = 6.4 |
| | | Hz, 2H), 2.67 (t, J = 6.0 Hz, 2H), |
| | | 2.00 (broad s, 2H), 1.87-1.78 (m, 2H), |
| | | 1.77-1.69 (m, 2H), 1.65 (broad s, |
| | | 4H), 1.49 (broad s, 2H). |
| 376 | 492.5 | 1H NMR (400 MHz, dmso) δ 9.02 (s, 1H), 8.82 |
| | | (s, 1H), 7.89 (d, J = 5.9 Hz, |
| | | 1H), 7.29 (d, J = 2.0 Hz, 1H), 7.25-7.18 |
| | | (m, 2H), 7.01 (dd, J = 8.8, 2.3 Hz, |
| | | 1H), 6.78 (d, J = 8.8 Hz, 1H), 6.72 (d, |
| | | J = 8.8 Hz, 1H), 6.04 (d, J = 5.9 Hz, |
| | | 1H), 4.18 (dd, J = 8.7, 5.2 Hz, 4H), 3.89 |
| | | (t, J = 6.3 Hz, 2H), 3.63 (s, 3H), 2.40 |
| | | (broad m, 6H), 1.82 (broad s, 2H), |
| | | 1.49 (broad s, 4H), 1.36 (broad s, 2H). |
| 377 | 507.5 | 1H NMR (500 MHz, DMSO) δ 9.46 (broad m, |
| | | 1H), 8.95 (broad s, 1H), 8.55 |
| | | (d, J = 0.5 Hz, 1H), 8.02 (m, 2H), 7.29 |
| | | (s, 1H), 7.22-7.11 (m, 1H), 6.92 (d, J = |
| | | 8.7 Hz, 1H), 6.24 (d, J = 5.8 Hz, 1H), |
| | | 4.01 (t, J = 5.8 Hz, 2H), 3.72 (s, 2H), |
| | | 3.67 (s, 3H), 3.51 (d, J = 11.8 Hz, 2H), |
| | | 3.26-3.19 (m, 2H), 3.08-2.87 (m, |
| | | 6H), 2.16-2.07 (m, 2H), 1.85 (d, J = 14.3 Hz, |
| | | 2H), 1.77-1.58 (m, 3H), 1.40 |
| | | (g, J = 13.3 Hz, 1H). |
| 378 | 507.4 | 1H NMR (500 MHz, DMSO) δ 9.40 (s, 1H), |
| | | 9.04 (s, 1H), 8.43 (s, 1H), 8.01 (d, |
| | | J = 5.7 Hz, 1H), 7.91 (d, J = 2.4 Hz, 1H), |
| | | 7.29 (s, 1H), 7.22 (dd, J = 8.7, 1.8 |
| | | Hz, 1H), 6.89 (d, J = 8.8 Hz, 1H), 6.17 (d, |
| | | J = 5.7 Hz, 1H), 3.97 (t, J = 6.1 Hz, |
| | | 2H), 3.68 (s, 3H), 3.05-2.99 (m, 2H), |
| | | 2.91 (broad s, 6H), 2.85-2.80 (m, 2H), |
| | | 2.12 (m, 2H), 2.01 (broad s, 2H), 1.66 |
| | | (broad s, 4H), 1.49 (broad s, 2H). |
| 379 | 504.6 | 1H NMR (500 MHz, DMSO) δ 9.39 (s, 1H), |
| | | 8.95 (s, 1H), 8.46 (s, 1H), 8.00 (d, |
| | | J = 5.7 Hz, 1H), 7.96 (d, J = 2.2 Hz, 1H), |
| | | 7.24 (s, 1H), 7.17 (d, J = 8.0 Hz, 1H), |
| | | 6.85 (d, J = 8.8 Hz, 1H), 6.17 (d, |
| | | J = 5.7 Hz, 1H), 3.94 (t, J = 6.4 Hz, 2H), 3.65 |
| | | (3, 2H), 3.43 (s, 2H), 2.83 (t, J = 5.9 Hz, |
| | | 2H), 2.68 (t, J = 6,0 Hz, 2H), 2.58- |
| | | 2.43 (m, 6H, overlaps with DMSO), |
| | | 2.36 (s, 3H), 1.93-1.82 (m, 2H), 1.58- |
| | | 1.49 (m, 4H), 1.45-1.36 (m, 2H). |
| 380 | 474.5 | 1H NMR (500 MHz, DMSO) δ 13.54 (s, 1H), |
| | | 9.46 (s, 1H), 8.94 (s, 1H), 8.66 |
| | | (s, 1H), 8.58 (d, J = 2.4 Hz, 1H), 8.00 (d, |
| | | J = 5.6 Hz, 2H), 7.29 (s, 1H), 7.17 (d, |
| | | J = 7.3 Hz, 1H), 6.80 (d, J = 8.7 Hz, |
| | | 1H), 6.18 (d, J = 5.7 Hz, 1H), 3.94-3.90 |
| | | (m, 2H), 3.62-3.57 (m, 3H), 2.42-2.25 |
| | | (m, 6H), 1.86-1.80 (m, 2H), 1.53- |
| | | 1.44 (m, 4H), 1.41-1.33 (m, 2H). |
| 381 | 474.4 | 1H NMR (500 MHz, DMSO) δ 11.52 (s, 1H), |
| | | 9.20 (s, 1H), 8.85 (s, 1H), 8.33 |
| | | (s, 1H), 8.27 (d, J = 2.3 Hz, 1H), 7.94 (d, |
| | | J = 5.7 Hz, 1H), 7.42 (d, J = 2.9 Hz, |
| | | 1H), 7.32 (s, 1H), 7.20-7.17 (m, 1H), 6.77 |
| | | (d, J = 8.7 Hz, 1H), 6.38-6.35 |
| | | (m, 1H), 6.12 (d, J = 5.8 Hz, 1H), |
| | | 3.92-3.89 (m, 2H), 3.56 (s, 3H), 2.41- |
| | | 2.32 (m, 6H), 1.84-1.80 (m, 2H), 1.51-1.47 |
| | | (m, 4H), 1.40-1.36 (m, 2H). |
| 382 | 493.4 | 1H NMR (500 MHz, DMSO) δ 9.64 (s, 1H), 8.94 |
| | | (s, 1H), 8.01 (d, J = 5.7 Hz, |
| | | 1H), 7.70 (d, J = 7.7 Hz, 1H), 7.33 (d, |
| | | J = 2.2 Hz, 1H), 7.27 (dd, J = 8.7, 2.4 |
| | | Hz, 1H). 7.21 (d, J = 8.5 Hz, 1H), 6.85 (d, |
| | | J = 8.7 Hz, 1H), 6.60 (d, J = 5.4 Hz, |
| | | 1H), 4.42-4.34 (m, 2H), 4.27-4.18 (m, 3H), |
| | | 3.93 (t, J = 6.4 Hz, 3H), 3.71 (s, |
| | | 3H), 2.38 (m, 6H), 1.86-1.78 (m, 2H), |
| | | 1.52-1.46 (m, 4H), 1.42-1.33 (m, |
| | | 2H). |
| 383 | 491.5 | 1H NMR (500 MHz, DMSO) δ 9.43 (s, 1H), |
| | | 9.07 (s, 1H), 8.33 (d, J = 1.9 Hz, |
| | | 1H), 8.07 (d, J = 5.7 Hz, 1H), 7.79 (d, |
| | | J = 1.9 Hz, 1H), 7.38 (s, 1H), 7.30 (dd, J = |
| | | 8.6, 2.0 Hz, 1H), 6.90 (d, J = 8.8 Hz, |
| | | 1H), 6.23 (d, J = 5.8 Hz, 1H), 4.24- |
| | | 4.16 (m, 2H), 4.00 (t, J = 6.4 Hz, 2H), |
| | | 3.74 (s, 3H), 2.86 (t, J = 6.5 Hz, 2H), |
| | | 2.42 (broad s, 6H), 2.14-2.02 (m, 2H), |
| | | 1.91 (broad s, 2H), 1.57 (broad s, 4H), |
| | | 1.46 (broad s, 2H). |
| 384 | 506.4 | 1H NMR (500 MHz, DMSO) δ 9.33 (s, 1H), |
| | | 8.88 (s, 1H), 8.28-8.26 (m, 1H). |
| | | 7.99 (d, J = 5.7 Hz, 1H), 7.42-7.40 (m, |
| | | 1H), 7.32 (s, 1H), 7.22 (s, 1H), 6.81 |
| | | (d, J = 8.7 Hz, 1H), 6.13 (d, J = 5.7 Hz, 1H), |
| | | 4.72 (s, 1H), 3,90 (t, J = 6.5 Hz, |
| | | 2H), 3.66 (s, 3H), 2.66-2.61 (m, 2H), |
| | | 2.40-2.27 (m, 6H), 1.84-1.79 (m, |
| | | 2H), 1.54 (s, 1H), 1.51-1.45 (m, 3H), 1.37 (s, 2H). |
| 385 | 519.6 | 1H NMR (500 MHz, DMSO) δ 9.42 (s, 1H), |
| | | 8.95 (s, 1H), 8.51 (d, J = 0.8 Hz, |
| | | 1H), 8.00 (d, J = 5.7 Hz, 1H), 7.98 (d, |
| | | J = 2.2 Hz, 1H), 7.25 (s, 1H), 7.14 (d, J = |
| | | 8.2 Hz, 1H), 6.84 (d, J = 8.7 Hz, 1H), |
| | | 6.17 (d, J = 5.8 Hz, 1H), 4.62 (s, 2H), |
| | | 3.93 (t, J = 6.5 Hz, 2H), 3.64 (s, 3H), |
| | | 2.70 (s, 2H), 2.43-2.23 (m, 6H), 1.87- |
| | | 1.78 (m, 2H), 1.49 (p, J = 5.4 Hz, 4H), |
| | | 1.43-1.30 (m, 2H), 1.23 (s, 6H). |
| 386 | 507.4 | 1H NMR (500 MHz, DMSO) δ 9.28 (s, 1H), |
| | | 8.98 (s, 1H), 8.02 (s, 1H), 7.98 (d, |
| | | J = 5.7 Hz, 1H), 7.79 (d, J = 1.9 Hz, 1H), |
| | | 7.32 (s, 1H), 7.24 (d, J = 8.3 Hz, 1H), |
| | | 6.83 (d, J = 8.7 Hz, 1H), 6.13 (d, |
| | | J = 5.7 Hz, 1H), 4.46-4.38 (m, 1H), 4.30 |
| | | (dd, J = 11.4, 2.3 Hz, 1H), 3.93 (t, |
| | | J = 6.4 Hz, 2H), 3.84 (dd, J = 11.4, 7.9 Hz, |
| | | 1H), 3.68 (s, 3H), 2.42 (s, 4H), 1.87 (s, 2H), |
| | | 1.53 (s, 4H), 1.41 (s, 2H), 1.34 (d, |
| | | J = 6.4 Hz, 3H). |
| 387 | 507.4 | 1H NMR (500 MHz, DMSO) δ 9.28 (s, 1H), |
| | | 8.98 (s, 1H), 8.02 (s, 1H), 7.98 (d, |
| | | J = 5.7 Hz, 1H), 7.79 (d, J = 1.9 Hz, 1H), |
| | | 7.32 (s, 1H), 7.24 (d, J = 8.3 Hz, 1H), |
| | | 6.83 (d, J = 8.7 Hz, 1H), 6.13 (d, |
| | | J = 5.7 Hz, 1H), 4.46-4.38 (m, 1H), 4.30 |
| | | (dd, J = 11.4, 2.3 Hz, 1H), 3.93 (t, |
| | | J = 6.4 Hz, 2H), 3.84 (dd, J = 11.4, 7.9 Hz, |
| | | 1H), 3.68 (s, 3H), 2.42 (s, 4H), 1.87 (s, 2H), |
| | | 1.53 (s, 4H), 1.41 (s, 2H), 1.34 (d, |
| | | J = 6.4 Hz, 3H). |
| 388 | 465.6 | 1H NMR (500 MHz, DMSO) δ 9.27 (s, 1H), |
| | | 8.98 (s, 1H), 7.95 (d, J = 5.7 Hz, |
| | | 2H), 7.79 (d, J = 2.3 Hz, 1H), 7.55 |
| | | (d, J = 9.0 Hz, 2H), 6.83-6.80 (m, 2H), |
| | | 6.10 (d, J = 5.7 Hz, 1H), 4.38-4.35 |
| | | (m, 2H), 4.27-4.24 (m, 2H), 3.96 (t, J = |
| | | 6.4 Hz, 2H), 3.60-3.54 (m, 4H), 2.42 (t, |
| | | J = 7.2 Hz, 2H), 2.36 (s, 4H), 1.88- |
| | | 1.83 (m, 2H). |
| 389 | 495.3 | 1H NMR (400 MHz, DMSO) δ 9.27 (s, 1H), |
| | | 8.96 (s, 1H), 8.00-7.94 (m, 2H), |
| | | 7.77 (d, J = 2.4 Hz, 1H), 7.31 (d, |
| | | J = 2.3 Hz, 1H), 7.24-7.19 (m, 1H), 6.82 (d, |
| | | J = 8.8 Hz, 1H), 6.12 (d, J = 5.8 Hz, |
| | | 1H), 4.38-4.33 (m, 2H), 4.26-4.22 (m, |
| | | 2H), 3.93 (t, J = 6.4 Hz, 2H), 3.67 (s, |
| | | 3H), 3.59-3.54 (m, 4H), 2.44-2.40 (m, |
| | | 2H), 2.36 (s, 4H), 1.86-1.80 (m, 2H). |
| 390 | 511.6 | 1H NMR (400 MHz, dmso) δ 9.52 (s, 1H), |
| | | 8.92 (s, 1H), 8.54 (s, 1H), 8.10 (s, |
| | | 1H), 7.97 (d, J = 5.7 Hz, 1H), 7.46 (d, |
| | | J = 9.0 Hz, 2H), 6.88-6.76 (m, 2H), |
| | | 6.14 (d, J = 5.7 Hz, 1H), 4.38 (s, 2H), 3.93 |
| | | (t, J = 6.3 Hz, 2H), 3.55-3.47 (m, |
| | | 6H), 3.37-3.28 (m, 2H overlap with water), |
| | | 2.37 (m, 6H), 1.90-1.72 (m, |
| | | 2H). |
| 391 | 539.5 | 1H NMR (500 MHz, DMSO) δ 9.58 (s, 1H), |
| | | 8.96 (s, 1H), 8.65 (s, 1H), 8.14 (s, |
| | | 1H), 8.04 (d, J = 5.7 Hz, 1H), 7.25 (s, 1H), |
| | | 7.20 (d, J = 9.0 Hz, 1H), 6.88 (d, J = |
| | | 8.7 Hz, 1H), 6,20 (d, J = 5.8 Hz, 1H), |
| | | 4.45 (s, 2H), 3.94 (t, J = 6.5 Hz, 2H), |
| | | 3.69 (s, 3H), 3.54 (t, J = 6.5 Hz, 2H), |
| | | 3.34 (t, J = 6.7 Hz, 23H), 2.40-2.35 (m, |
| | | 2H), 2.32 (s, 4H), 1.87-1.78 (m, 2H), |
| | | 1.52-1.45 (m, 4H), 1.41-1.32 (m, |
| | | 2H). |
| 392 | 508.4 | 1H NMR (500 MHz, DMSO) δ 9.98 (s, 1H), |
| | (M-H) | 9.07 (s, 1H), 9.02 (s, 2H), 8.22 (s, |
| | | 1H), 8.06 (d, J = 5.6 Hz, 1H), 8.01 (d, |
| | | J = 8.6 Hz, 1H), 7.80 (dd, J = 8.6, 1.8 |
| | | Hz, 1H), 7.20 (s, 1H), 7.17 (d, J = 8.2 Hz, 1H), |
| | | 6.85 (d, J = 8.7 Hz, 1H), 6.26 |
| | | (d, J = 5.7 Hz, 1H), 3.92 (t, J = 6.4 Hz, |
| | | 2H), 3.60 (s, 3H), 2.34 (t, J = 7.2 Hz, |
| | | 2H), 2.27 (s, 4H), 1.84-1.76 (m, 2H), 1.41 |
| | | (dt, J = 10.9, 5.5 Hz, 4H), 1.31 (d, |
| | | J = 5.1 Hz, 2H). |
| 393 | 521.3 | 1H NMR (500 MHz, DMSO) δ 9.96 (s, 1H), |
| | | 9.10 (s, 1H), 9.08 (s, 1H), 9.05 (s, |
| | | 1H), 8.12 (t, J = 6.5 Hz, 1H), 7.69 (dd, |
| | | J = 16.9, 9.5 Hz, 1H), 7.63 (s, 1H), 7.29 |
| | | (s, 1H), 7.23 (d, J = 8.2 Hz, 1H), 6.88 |
| | | (d, J = 8.7 Hz, 1H), 6.31 (d, J = 5.7 Hz, |
| | | 1H), 3.96 (t, J = 6.5 Hz, 2H), 3.70-3.62 |
| | | (m, 3H), 2.39 (t, J = 7.1 Hz, 2H), 2.33 |
| | | (s, 4H), 1.91-1.81 (m, 2H), 1.53-1.45 (m, 4H), |
| | | 1.38 (d, J = 5.0 Hz, 2H). |
| 394 | 457.4 | 1H NMR (500 MHz, DMSO) δ 9.73 (s, 1H), |
| | | 9.01 (s, 1H), 8.85 (t, J = 7.7 Hz, |
| | | 2H), 8.00 (d, J = 5.7 Hz, 1H), 7.86 (d, |
| | | J = 8.0 Hz, 1H), 7.68 (s, 1H), 7.50 (dtd, |
| | | J = 14.7, 6.8, 1.5 Hz, 4H), 6.84-6.77 |
| | | (m, 2H), 6.22 (d, J = 5.7 Hz, 1H), 3.93 |
| | | (t, J = 6.4 Hz, 2H), 3.53-3.49 (m, 4H), 2.37 |
| | | (t, J = 7.2 Hz, 2H), 2.31 (s, 4H), |
| | | 1.86-1.79 (m, 2H). |
| 395 | 525.3 | 1H NMR (400 MHz, DMSO) δ 10.08 (s, 1H), |
| | | 9.21-8.98 (m, 3H), 8.25 (s, |
| | | 1H), 8.12 (d, J = 5.7 Hz, 1H), 7.95 (s, 1H), |
| | | 7.81 (d, J = 8.2 Hz, 1H), 7.56 (d, J = |
| | | 8.0 Hz, 2H), 6.90 (d, J = 8.8 Hz, 2H), |
| | | 6.33 (d, J = 5.5 Hz, 1H), 4.06-3.98 |
| | | (m, 2H), 3.61-3.53 (m, 4H), 2.47-2.32 (m, 6H), |
| | | 1.96-1.85 (m, 2H). |
| 396 | 482.3 | 1H NMR (500 MHz, DMSO) δ 10.03 (s, 1H), |
| | | 9.08 (d, J = 13.0 Hz, 3H), 8.27 |
| | | (s, 1H), 8.12 (d, J = 5.6 Hz, 1H), 8.08 (d, |
| | | J = 8.6 Hz, 1H), 7.87 (dd, J = 8.6, 1.9 |
| | | Hz, 1H), 7.55 (d, J = 8.7 Hz, 2H), 6.92 (d, |
| | | J = 9.0 Hz, 2H), 6.32 (d, J = 5.7 Hz, |
| | | 1H), 4.03 (t, J = 6.4 Hz, 2H), 3.61-3.54 (m, 4H), |
| | | 2.45 (t, J = 7.2 Hz, 2H), 2.37 |
| | | (s, 4H), 1.91 (dt, J = 13.4, 6.7 Hz, 2H). |
| 397 | 485.3 | 1H NMR (500 MHz, DMSO) δ 9.75 (s, 1H), |
| | | 9.06 (s, 1H), 8.88 (s, 2H), 8.06 (d, |
| | | J = 5.7 Hz, 1H), 7.73 (s, 1H), 7.70 (s, 1H), |
| | | 7.56 (d, J = 8.4 Hz, 2H), 7.44 (d, J = |
| | | 7.0 Hz, 1H), 6.87 (d, J = 9.0 Hz, 2H), 6.27 |
| | | (d, J = 5.7 Hz, 1H), 4.00 (t, J = 6.4 |
| | | Hz, 2H), 3.62-3.55 (m, 4H), 2.81 |
| | | (dd, J = 15.1, 7.6 Hz, 2H), 2.47-2.42 (m, |
| | | 2H), 2.38 (s, 4H), 1.90 (dd, J = 13.8, 6.7 Hz, |
| | | 2H), 1.29 (t, J = 7.6 Hz, 3H). |
| 398 | 539.6, | 1H NMR (500 MHz, DMSO) δ 9.95 (s, 1H), |
| | 541.7 | 9.12 (s, 1H), 9.06 (s, 1H), 9.00 (s, |
| | | 1H), 8.09 (d, J = 5.7 Hz, 1H), 7.77-7.72 |
| | | (m, 2H), 7.58-7.51 (m, 3H), 6.90- |
| | | 6.87 (m, 2H), 6.31 (d, J = 5.7 Hz, 1H), |
| | | 4.00 (t, J = 6.3 Hz, 2H), 3.11-3.06 (m, |
| | | 4H), 2.94-2.89 (m, 4H), 2.64 (t, |
| | | J = 7.1 Hz, 2H), 1.91-1.86 (m, 2H). |
| 399 | 539.7, | 1H NMR (500 MHz, DMSO) δ 9.89 (s, 1H), |
| | 541.7 | 9.09 (s, 1H), 9.01-8.94 (m, 2H), |
| | | 8.08 (d, J = 5.7 Hz, 1H), 7.98 (d, |
| | | J = 2.1 Hz, 1H), 7.79 (s, 1H), 7.61-7.54 (m, |
| | | 3H), 6.90-6.86 (m, 2H), 6.29 (d, J = 5.7 Hz, |
| | | 1H), 4.00 (t, J = 6.4 Hz, 2H), |
| | | 3.10-3.06 (m, 4H), 2.95-2.90 (m, 4H), |
| | | 2.64 (t, J = 7.1 Hz, 2H), 1.91-1.86 |
| | | (m, 2H). |
| 400 | 533.4 | 1H NMR (500 MHz, DMSO) δ 9.68 (s, 1H), |
| | | 9.05 (s, 1H), 8.87-8.80 (m, 2H), |
| | | 8.03 (d, J = 5.7 Hz, 1H), 7.68 (d, J = 8.5 |
| | | Hz, 1H), 7.55 (d, J = 8.8 Hz, 2H), 7.34 |
| | | (d, J = 2.5 Hz, 1H), 7.25-7.22 (m, 1H), |
| | | 6.87-6.83 (m, 2H), 6.25 (d, J = 5.7 |
| | | Hz, 1H), 3.98 (t, J = 6.4 Hz, 2H), 3.90 |
| | | (s, 3H), 3.10-3.07 (m, 4H), 2.93-2.89 |
| | | (m, 4H), 2.65-2.61 (m, 2H), 1.91-1.85 (m, 2H). |
| 401 | 491.4, | 1H NMR (500 MHz, Acetone) δ 9.09 (s, 1H), |
| | 493.4 | 9.03 (s, 1H), 8.88 (s, 1H), 8.35 |
| | | (s, 1H), 8.09 (d, J = 5.6 Hz, 1H), 7.95 (d, |
| | | J = 9.0 Hz, 1H), 7.73 (s, 1H), 7.65- |
| | | 7.62 (m, 2H), 7.56-7.53 (m, 1H), 6.95 (d, |
| | | J = 8.6 Hz, 2H), 6.33 (d, J = 5.6 Hz, |
| | | 1H), 4.11 (t, J = 6.3 Hz, 2H), 3.63-3.59 (m, |
| | | 4H), 2.51 (t, J = 7.1 Hz, 2H), 2.42 |
| | | (s, 4H), 2.00-1.95 (m, 2H). |
| 402 | 555.3 | 1H NMR (400 MHz, DMSO) δ 9.81 (s, 1H), |
| | | 9.05 (s, 1H), 8.95-8.89 (m, 2H), |
| | | 8.07 (d, J = 5.7 Hz, 1H), 7.90 (d, |
| | | J = 8.0 Hz, 1H), 7.77-7.68 (m, 1H), 7.61- |
| | | 7.50 (m, 2H), 7.28 (s, 1H), 7.19 (d, J = 8.5 |
| | | Hz, 1H), 6.87 (d, J = 8.7 Hz, 1H), |
| | | 6.28 (d, J = 5.7 Hz, 1H), 4.05-3.99 (m, |
| | | 1H), 3.96 (t, J = 6.3 Hz, 2H), 3.79- |
| | | 3.66 (m, 3H), 3.62-3.55 (m, 1H), 2.43-2.37 |
| | | (m, 2H), 2.36-2.27 (m, 4H), |
| | | 1.90-1.71 (m, 5H), 1.61-1.52 (m, 1H), |
| | | 1.51-1.43 (m, 4H), 1.40-1.32 (m, |
| | | 2H). |
| 403 | 493.4 | 1H NMR (500 MHz, MeOD) (broad |
| | | peaks) δ 8.98 (s, 1H), 8.85 (s, 1H), 7.99 |
| | | (s, 1H), 7.55-7.37 (m, 4H), 6.92 (s, 2H), |
| | | 6.25 (s, 1H), 4.06 (s, 2H), 3.72 (s, |
| | | 4H), 2.65-2.45 (m, 6H), 2.03 (s, 2H). |
| 404 | 541.3 | 1H NMR (500 MHz, DMSO) δ 9.98 (s, 1H), |
| | | 9.14 (s, 1H), 9.08 (s, 1H), 9.02 (s, |
| | | 1H), 8.11 (d, J = 5.6 Hz, 1H), 7.78 |
| | | (s, 1H), 7.65-7.46 (m, 4H), 6.92-6.85 |
| | | (m, 2H), 6.32 (d, J = 5.7 Hz, 1H), 4.00 |
| | | (t, J = 6.4 Hz, 2H), 3.59-3.57 (m, 4H), |
| | | 2.44 (t, J = 7.2 Hz, 2H), |
| | | 2.38 (s, 4H), 1.93-1.85 (m, 2H). |
| 405 | 497.4 | 1H NMR (400 MHz, dmso) δ 9.66 (s, 1H), 9.00 |
| | | (s, 1H), 8.80 (s, 1H), 8.00 (d, J = |
| | | 3.7 Hz, 1H), 7.57 (s, 2H), 7.50 (d, J = 7.3 |
| | | Hz, 2H), 7.22 (d, J = 8.4 Hz, 1H), |
| | | 6.81 (d, J = 7.0 Hz, 2H), 6.21 (d, J = 5.7 Hz, |
| | | 1H), 3.94 (t, J = 5.6 Hz, 2H), 3.53 |
| | | (s, 4H), 2.39 (s, 2H), 2.33 (s, 4H), 2.07 |
| | | (s, 1H), 1.88-1.78 (m, 2H), 1.00 (d, J = |
| | | 8.2 Hz, 2H), 0.77 (d, J = 4.8 Hz, 2H). |
| 406 | 487.5 | 1H NMR (500 MHz, DMSO) δ 9.68 (s, 1H), |
| | | 9.05 (s, 1H), 8.86 (d, J = 17.2 Hz, |
| | | 2H), 8.04 (d, J = 5.7 Hz, 1H), 7.68 (s, 1H), |
| | | 7.55 (d, J = 8.5 Hz, 2H), 7.34 (d, J = |
| | | 2.5 Hz, 1H), 7.24-7.22 (m, 1H), 6.87-6.81 |
| | | (m, 2H), 6.24 (d, J = 5.7 Hz, |
| | | 1H), 3.98 (t, J = 6.4 Hz, 2H), 3.90 (s, 3H), |
| | | 3.60-3.55 (m, 4H), 2.45-2.41 (m, |
| | | 2H), 2.37 (s, 4H), 1.90-1.86 (m, 2H). |
| 407 | 471.6 | 1H NMR (400 MHz, dmso) δ 9.73 (s, 1H), |
| | | 9.05 (s, 1H), 8.86 (s, 2H), 7.72 (s, |
| | | 1H), 7.66 (d, J = 7.7 Hz, 1H), 7.55 |
| | | (d, J = 8.9 Hz, 2H), 7.41-7.38 (m, 1H), |
| | | 6.86 (d, J = 9.0 Hz, 2H), 6.26 (s, 1H), |
| | | 4.01-3.97 (m, 2H), 3.60-3.56 (m, |
| | | 4H), 2.49 (s, 3H), 2.47-2.42 (m, 2H), |
| | | 2.38 (s, 4H), 1.91-1.86 (m, 2H). |
| 408 | 473.3 | 1H NMR (500 MHz, DMSO) δ 9.93 (s, 1H), |
| | | 9.61 (s, 1H), 9.03 (s, 1H), 8.77 (d, |
| | | J = 7.0 Hz, 2H), 8.02 (d, J = 5.7 Hz, 1H), |
| | | 7.62 (d, J = 11.2 Hz, 1H), 7.55 (d, J = |
| | | 8.7 Hz, 2H), 7.20 (d, J = 2.3 Hz, 1H), 7.13 |
| | | (dd, J = 8.8, 2.4 Hz, 1H), 6.84 (d, J = |
| | | 9.0 Hz, 2H), 6.23 (d, J = 5.7 Hz, 1H), |
| | | 3.98 (t, J = 6.4 Hz, 2H), 3.58 (t, J = 4.5 |
| | | Hz, 4H), 2.44 (t, J = 7.1 Hz, 2H), |
| | | 2.38 (s, 4H), 1.92-1.85 (m, 2H). |
| 409 | 535.1, | 1H NMR (500 MHz, DMSO) δ 9.95 (s, 1H), |
| | 537.1 | 9.16 (s, 1H), 9.04 (s, 1H), 8.98 (s, |
| | | 1H), 8.18 (s, 1H), 8.13 (d, J = 5.7 Hz, 1H), |
| | | 7.75 (s, 2H), 7.59 (d, J = 7.4 Hz, |
| | | 2H), 6.92 (d, J = 9.0 Hz, 2H), 6.33 (d, |
| | | J = 5.7 Hz, 1H), 4.04 (t, J = 6.4 Hz, 2H), |
| | | 3.65-3.58 (m, 4H), 2.51-2.45 (m, 2H), |
| | | 2.42 (s, 4H), 1.97-1.89 (m, 2H). |
| 410 | 491.6, | 1HNMR(500 MHz, DMSO) δ 9.90 (s, |
| | 493.6 | 1H), 9.11 (s, 1H), 9.00 (s, 1H), 8.95 (s, |
| | | 1H), 8.08 (d, J = 5.6 Hz, 1H), 7.98 |
| | | (d, J = 2.0 Hz, 1H), 7.79 (s, 1H), 7.61- |
| | | 7.52 (m, 3H), 6.90-6.85 (m, 2H), 6.29 (d, |
| | | J = 5.7 Hz, 1H), 3.99 (t, J = 6.4 Hz, |
| | | 2H), 3.62-3.54 (m, 4H), 2.46-2.41 |
| | | (m, 2H), 2.38 (s, 4H), 1.92-1.86 (m, |
| | | 2H). |
| 411 | 535.4 | 1H NMR (400 MHz, dmso) δ 9.98 (s, 1H), |
| | | 9.28 (s, 1H), 9.14 (s, 1H), 9.02 (d, J = |
| | | 2.6 Hz, 1H), 8.46 (s, 1H), 8.11 (d, J = 8.8 Hz, |
| | | 1H), 8.06 (d, J = 5.7 Hz, 1H), |
| | | 7.97 (dd, J = 8.8, 2.1 Hz, 1H), 7.56 (d, |
| | | J = 8.9 Hz, 2H), 6.83 (t, J = 6.2 Hz, 2H), |
| | | 6.28 (d, J = 5.7 Hz, 1H), 3.94 (t, |
| | | J = 6.4 Hz, 2H), 3.55-3.48 (m, 4H), 2.41- |
| | | 2.35 (m, 2H), 2.32 (s, 4H), 1.86-1.78 (m, 2H). |
| | | * one H overlap with H2O |
| | | peak. |
| 412 | 521.6, | 1HNMR(500 MHz, DMSO) δ 10.00 (s, 1H), |
| | 523.9 | 9.15 (s, 1H), 9.08 (s, 2H), 8.11 (d, |
| | | J = 5.7 Hz, 1H), 7.77-7.75 (m, 1H), 7.52 |
| | | (d, J = 7.4 Hz, 1H), 7.23 (s, 3H), |
| | | 6.90 (d, J = 8.7 Hz, 1H), 6.32 (d, J = 5.7 |
| | | Hz, 1H), 3.98 (t, J = 6.4 Hz, 2H), 3.66 |
| | | (s, 3H), 3.59-3.56 (m, 2H), 2.65-2.63 |
| | | (m, 1H), 2.45-2.42 (m, 2H), 2.41- |
| | | 2.35 (m, 4H), 2.09-2.08 (m, 1H), |
| | | 1.89-1.85 (m, 2H). |
| 413 | 521.4, | 1HNMR(400 MHz, DMSO) δ 9.98 (s, 1H), |
| | 523.3 | 9.12 (s, 1H), 9.08 (s, 1H), 9.03 (s, |
| | | 1H), 8.11 (d, J = 5.7 Hz, 1H), 7.75 (t, |
| | | J = 7.4, 1.2 Hz, 2H), 7.51 (d, J = 7.7 Hz, |
| | | 1H), 7.29 (s, 1H), 7.23 (d, J = 7.8 Hz, |
| | | 1H), 6.89 (d, J = 8.7 Hz, 1H), 6.32 (s, |
| | | 1H), 3.98 (t, J = 6.4 Hz, 2H), 3.66 |
| | | (s, 3H), 3.59-3.55 (m, 4H), 2.46-2.35 (m, |
| | | 6H), 1.90-1.85 (m, 2H). |
| 414 | 473.4 | 1H NMR (500 MHz, DMSO) δ 9.77 (s, 1H), |
| | | 9.19 (s, 1H), 8.82 (s, 1H), 8.75 (d, |
| | | J = 2.3 Hz, 1H), 8.14 (d, J = 5.7 Hz, 1H), |
| | | 7.84 (d, J = 9.0 Hz, 1H), 7.68 (d, J = |
| | | 8.8 Hz, 2H), 7.23 (dd, J = 9.0, 2.7 Hz, 1H), |
| | | 7.11 (s, 1H), 7.00-6.92 (m, 2H), |
| | | 6.36 (d, J = 5.7 Hz, 1H), 4.08 (t, J = 6.4 Hz, |
| | | 2H), 3.66 (t, J = 4.6 Hz, 4H), 2.54- |
| | | 2.49 (m, 2H), 2.46 (s, 4H), 2.02-1.91 (m, 2H). |
| 415 | 525.3 | 1H NMR (400 MHz, DMSO) δ 9.93 (s, 1H), |
| | | 9.08 (s, 2H), 8.97 (s, 1H), 8.05 (d, |
| | | J = 5.7 Hz, 1H), 7.99 (s, 1H), 7.94 (d, J = 7.3 Hz, |
| | | 1H), 7.63 (t, J = 7.7 Hz, 1H), |
| | | 7.51 (d, J = 8.8 Hz, 2H), 6.86-6.81 (m, 2H), |
| | | 6.27 (d, J = 5.7 Hz, 1H), 3.94 (t, |
| | | J = 6.4 Hz, 2H), 3.55-3.50 (m, 4H), 2.39 |
| | | (t, J = 6.7 Hz, 2H), 2.34-2.30 (m, |
| | | 4H), 1.89-1.76 (m, 2H). |
| 416 | 491.6, | 1HNMR(500 MHz, DMSO) δ 9.96 (s, 1H), |
| | 493.5 | 9.11 (s, 1H), 9.05 (s, 1H), 9.00 (s, |
| | | 1H), 8.09 (d, J = 5.6 Hz, 1H), 7.75 |
| | | (d, J = 8.2 Hz, 2H), 7.57-7.51 (m, 3H), |
| | | 6.88 (d, J = 8.9 Hz, 2H), |
| | | 6.31 (d, J = 5.7 Hz, |
| | | 1H), 3.99 (t, J = 6.3 Hz, 2H), 3.59- |
| | | 3.56 (m, 4H), 2.45-2.42 (m, 2H), 2.37 |
| | | (s, 4H), 1.91-1.86 (m, 2H). |
| 417 | 485.5 | 1H NMR (500 MHz, DMSO) δ 9.84 (s, 1H), |
| | | 9.11 (s, 1H), 8.94 (s, 1H), 8.86 (d, |
| | | J = 2.2 Hz, 1H), 8.11 (d, J = 5.7 Hz, 1H), |
| | | 7.62-7.56 (m, 2H), 7.49 (t, J = 8.0 |
| | | Hz, 1H), 7.33 (s, 1H), 7.06 (dd, J = 7.8, |
| | | 0.9 Hz, 1H), 6.96-6.89 (m, 2H), 6.32 |
| | | (d, J = 5.7 Hz, 1H), 4.06-4.01 (m, 2H), |
| | | 4.00-3.97 (s, 3H), 3.61 (dd, J = 9.1, |
| | | 4.5 Hz, 4H), 2.47 (dd, J = 14.8, 7.4 Hz, 2H), |
| | | 2.43 (d, J = 9.2 Hz, 4H), 1.93 (dt, |
| | | J = 11.5, 5.5 Hz, 2H). |
| 418 | 569.3 | 1H NMR (400 MHz, DMSO) δ 9.83 (s, 1H), |
| | | 9.08 (s, 1H), 8.91 (s, 2H), 8.07 (d, |
| | | J = 5.7 Hz, 1H), 7.90 (d, J = 8.1 Hz, |
| | | 1H), 7.76-7.64 (m, 1H), 7.60-7.48 (m, |
| | | 2H), 7.29 (s, 1H), 7.19-7.09 (m, 1H), 6.87 |
| | | (d, J = 8.7 Hz, 1H), 6.27 (d, J = 5.7 |
| | | Hz, 1H), 3.95 (t, J = 6.2 Hz, 2H), |
| | | 3.81-3.71 (m, 2H), 3.64-3.51 (m, 2H), |
| | | 3.20 (t, J = 11.4 Hz, 2H), 2.39 (t, |
| | | J = 7.2 Hz, 2H), 2.35-2.22 (m, 4H), 1.88- |
| | | 1.73 (m, 3H), 1.54-1.40 (m, 6H), |
| | | 1.40-1.29 (m, 2H), 1.24-1.07 (m, 2H). |
| 419 | 555.3 | 1H NMR (500 MHz, DMSO) δ 10.13 (s, 1H), |
| | | 9.34 (s, 1H), 9.20 (s, 1H), 9.16 |
| | | (d, J = 2.5 Hz, 1H), 8.31 (s, 1H), 8.25-8.19 |
| | | (m, 2H), 7.91 (dd, J = 8.8, 2.0 Hz, |
| | | 1H), 7.41 (s, 1H), 7.36 (d, J = 8.5 Hz, 1H), |
| | | 6.98 (d, J = 8.7 Hz, 1H), 6.43 (d, J = |
| | | 5.7 Hz, 1H), 4.05 (t, J = 6.5 Hz, 2H), |
| | | 3.76 (s, 3H), 3.69-3.64 (m, 4H), 2.52 |
| | | (t, J = 7.2 Hz, 2H), 2.46 (s, 4H), |
| | | 1.99-1.91 (m, 2H). |
| 420 | 567.4 | 1H NMR (400 MHz, DMSO) δ 9.84 (s, 1H), |
| | | 9.08 (s, 1H), 8.95-8.84 (m, 2H), |
| | | 8.06 (d, J = 5.7 Hz, 1H), 7.90 (d, |
| | | J = 8.1 Hz, 1H), 7.75-7.65 (m, 1H), 7.60- |
| | | 7.48 (m, 2H), 7.29 (s, 1H), 7.18-7.08 (m, 1H), |
| | | 6.86 (d, J = 8.7 Hz, 1H), 6.28 |
| | | (d, J = 5.7 Hz, 1H), 3.95 (t, J = 6.1 Hz, |
| | | 2H), 3.51 (s, 2H), 2.46-2.26 (m, 5H), |
| | | 1.94-1.79 (m, 2H), 1.74-1.28 (m, 14H), |
| | | 1.22-0.98 (m, 3H), 0.89-0.72 |
| | | (m, 1H). |
| 421 | 563.5 | 1H NMR (500 MHz, MeOD) δ 8.92 (d, |
| | | J = 2.3 Hz, 1H), 8.67 (d, J = 2.6 Hz, |
| | | 1H), 7.92 (d, J = 5.8 Hz, 1H), 7.72 (d, |
| | | J = 8.9 Hz, 2H), 7.57 (dd, J = 8.9, 2.2 |
| | | Hz, 1H), 7.10 (d, J = 2.3 Hz, 1H), 7.01 (dd, |
| | | J = 8.5, 2.4 Hz, 1H), 6.86 (d, J = |
| | | 8.6 Hz, 1H), 6.18 (d, J = 5.8 Hz, 1H), |
| | | 4.02 (t, J = 6.1 Hz, 2H), 3.62 (s, 3H), |
| | | 2.66-2.58 (m, 2H), 2.53 (s, 4H), 1.98 (dd, |
| | | J = 9.6, 6.0 Hz, 2H), 1.57 (dt, J = |
| | | 11.2, 5.7 Hz, 4H), 1.43 (s, 2H). |
| 422 | 519.6 | 1H NMR (500 MHz, DMSO) δ 9.96 (s, 1H), |
| | | 9.43 (s, 1H), 9.08 (s, 1H), 8.66 (d, |
| | | J = 1.9 Hz, 1H), 8.12 (d, J = 5.6 Hz, 1H), |
| | | 7.95 (d, J = 8.4 Hz, 1H), 7.72 (dd, J = |
| | | 7.5, 1.0 Hz, 1H), 7.59 (dd, J = 8.4, |
| | | 7.6 Hz, 1H), 7.32 (s, 1H), 7.23-7.14 (m, |
| | | 1H), 6.81 (d, J = 8.7 Hz, 1H), 6.31 (d, |
| | | J = 5.7 Hz, 1H), 3.93 (t, J = 6.3 Hz, 2H), |
| | | 3.56 (s, 3H), 2.45-2.30 (m, 4H), 1.85 (s, 2H), |
| | | 1.51 (s, 4H), 1.40 (s, 2H). * one |
| | | peak missing due to overlap. |
| 423 | 503.4 | 1H NMR (500 MHz, DMSO) δ 9.92 (s, 1H), |
| | | 9.20 (s, 1H), 9.11 (s, 1H), 8.75 (d, |
| | | J = 1.9 Hz, 1H), 8.12 (d, J = 5.6 Hz, 1H), |
| | | 7.81 (d, J = 8.5 Hz, 1H), 7.61-7.55 |
| | | (m, 1H), 7.43-7.35 (m, 1H), 7.28 (s, 1H), 7.24 |
| | | (dd, J = 8.6, 2.4 Hz, 1H), 6.82 |
| | | (d, J = 8.7 Hz, 1H), 6.31 (d, J = 5.7 Hz, |
| | | 1H), 3.93 (t, J = 6.5 Hz, 2H), 3.60 (s, |
| | | 3H), 2.41-2.36 (m, 2H), 2.33 (s, 4H), |
| | | 1.87-1.80 (m, 2H), 1.49 (dt, J = 11.0, |
| | | 5.6 Hz, 4H), 1.38 (d, J = 5.7 Hz, 2H). |
| 424 | 499.5 | 1H NMR (500 MHz, MeOD) δ 8.47 |
| | | (d, J = 6.5 Hz, 1H), 7.92-7.87 (m, 2H), |
| | | 7.70 (d, J = 8.4 Hz, 1H), 7.62 (ddd, |
| | | J = 8.4, 6.9, 1.4 Hz, 1H), 7.49-7.44 (m, |
| | | 1H), 7.04 (d, J = 2.4 Hz, 1H), 6.87 (dd, |
| | | J = 8.6, 2.5 Hz, 1H), 6.56 (d, J = 8.7 |
| | | Hz, 1H), 6.23 (d, J = 5.9 Hz, 1H), 3.88 (t, |
| | | J = 6.0 Hz, 2H), 3.36 (s, 3H), 2.67 (s, |
| | | 4H), 2.62 (s, 3H), 1.98-1.90 (m, 2H), |
| | | 1.65-1.58 (m, 4H), 1.47 (s, 2H), 1.22 |
| | | (s, 2H). |
| 425 | 525.4 | |
| 426 | 515.2 | 1H NMR (500 MHz, DMSO) δ 9.68 (s, 1H), |
| | | 9.03 (s, 1H), 8.88 (d, J = 2.3 Hz, |
| | | 1H), 8.86 (s, 1H), 8.06 (d, J = 5.7 Hz, 1H), |
| | | 7.68 (d, J = 6.6 Hz, 1H), 7.34 (d, J = |
| | | 2.5 Hz, 1H), 7.30 (s, 1H), 7.23 (dd, J = 8.9, |
| | | 2.5 Hz, 2H), 6.86 (d, J = 8.7 Hz, |
| | | 1H), 6.27 (s, 1H), 3.96 (t, J = 6.4 Hz, 2H), |
| | | 3.91 (s, 3H), 3.63 (s, 3H), 2.42 (t, J = |
| | | 7.2 Hz, 2H), 2.37 (s, 4H), 1.90-1.82 |
| | | (m, 2H), 1.54-1.47 (m, 4H), 1.39 (s, |
| | | 2H). |
| 427 | 503.4 | 1H NMR (500 MHz, DMSO) δ 9.91 (s, 1H), |
| | | 9.08 (s, 1H), 9.00 (s, 1H), 8.90 (d, |
| | | J = 2.1 Hz, 1H), 8.11 (d, J = 5.7 Hz, 1H), |
| | | 7.98 (dd, J = 9.1, 5.6 Hz, 1H), 7.47 |
| | | (td, J = 8.8, 2.8 Hz, 1H), 7.43 (s, 1H), 7.29 |
| | | (s, 1H), 7.21 (d, J = 7.4 Hz, 1H), |
| | | 6.91 (d, J = 8.7 Hz, 1H), 6.32 (d, J = 5.7 Hz, |
| | | 1H), 3.98 (t, J = 6.5 Hz, 2H), 3.66 |
| | | (s, 3H), 2.40 (dd, J = 15.1, 7.8 Hz, 2H), |
| | | 2.34 (s, 4H), 1.90-1.82 (m, 2H), 1.53- |
| | | 1.45 (m, 4H), 1.38 (d, J = 5.6 Hz, 2H). |
| 428 | 519.9 | 1H NMR (500 MHz, DMSO) δ 9.91 (s, 1H), |
| | | 9.10 (s, 1H), 9.03 (s, 1H), 8.97 (d, |
| | | J = 2.2 Hz, 1H), 8.11 (d, J = 5.7 Hz, 1H), |
| | | 7.98 (d, J = 2.0 Hz, 1H), 7.79 (s, 1H), |
| | | 7.59 (dd, J = 8.7, 2.0 Hz, 1H), 7.29 (s, 1H), |
| | | 7.23 (d, J = 8.4 Hz, 1H), 6.89 (d, J = |
| | | 8.7 Hz, 1H), 6.31 (d, J = 5.7 Hz, 1H), |
| | | 3.97 (t, J = 6.5 Hz, 2H), 3.66 (s, 3H), |
| | | 2.42 (t, J = 7.2 Hz, 2H), 2.36 (s, 4H), |
| | | 1.92-1.83 (m, 2H), 1.51 (dt, J = 10.9, |
| | | 5.5 Hz, 4H), 1.39 (d, J = 5.1 Hz, 2H). |
| 429 | 499.5 | 1H NMR (500 MHz, DMSO) δ 9.85 (s, 1H), |
| | | 9.08 (s, 1H), 8.90 (s, 1H), 8.84 (s, |
| | | 1H), 8.08 (d, J = 5.7 Hz, 1H), 7.81 (d, |
| | | J = 8.5 Hz, 1H), 7.50 (s, 1H), 7.42 (dd, J = |
| | | 8.5, 1.7 Hz, 1H), 7.28 (s, 1H), 7.23 (d, |
| | | J = 8.2 Hz, 1H), 6.90 (d, J = 8.7 Hz, |
| | | 1H), 6.30 (d, J = 5.7 Hz, 1H), 3.98 (t, |
| | | J = 6.4 Hz, 2H), 3.65 (s, 3H), 2.49 (s, |
| | | 3H), 2.48-2.44 (m, 2H), 2.40 (s, 4H), |
| | | 1.92-1.84 (m, 2H), 1.54-1.48 (m, |
| | | 4H), 1.42-1.36 (m, 2H). |
| 430 | 519.5 | 1H NMR (500 MHz, DMSO) δ 9.98 (s, 1H), |
| | | 9.13 (s, 1H), 9.08 (s, 1H), 9.04 (s, |
| | | 1H), 8.12 (d, J = 5.7 Hz, 1H), 7.76 (d, J = 7.4 |
| | | Hz, 1H), 7.72 (s, 1H), 7.53 (t, J = |
| | | 7.8 Hz, 1H), 7.30 (s, 1H), 7.24 (d, J = 8.6 Hz, |
| | | 1H), 6.90 (d, J = 8.7 Hz, 1H), |
| | | 6.33 (d, J = 5.7 Hz, 1H), 3.97 (t, J = 6.5 |
| | | Hz, 2H), 3.67 (s, 3H), 2.41 (t, J = 7.2 |
| | | Hz, 2H), 2.35 (s, 4H), 1.90-1.83 (m, 2H), 1.50 (dt, |
| | | J = 10.8, 5.4 Hz, 4H), 1.38 |
| | | (s, 2H). |
| 431 | 503.4 | 1H NMR (500 MHz, DMSO): δ 9.79 (s, 1H), |
| | | 9.15 (d, J = 2.5 Hz, 1H), 9.10 (s, |
| | | 1H), 8.92 (d, J = 1.4 Hz, 1H), 8.17 (d, |
| | | J = 3.6 Hz, 1H), 7.95 (d, J = 8.3 Hz, |
| | | 1H), 7.79 (d, J = 8.0 Hz, 1H), 7.63 |
| | | (ddd, J = 8.4, 6.9, 1.5 Hz, 1H), 7.60-7.54 |
| | | (m, 1H), 7.22 (d, J = 1.8 Hz, 1H), |
| | | 7.18 (dd, J = 8.6, 2.2 Hz, 1H), 6.82 (d, J = |
| | | 8.7 Hz, 1H), 3.94 (t, J = 6.5 Hz, 2H), |
| | | 3.59 (s, 3H), 2.42-2.35 (m, 2H), 2.32 (s, |
| | | 4H), 1.90-1.78 (m, 2H), 1.54-1.44 (m, 4H), 1.38 |
| | | (dd, J = 11.7, 6.3 Hz, 2H). |
| 432 | 473.5 | 1H NMR (500 MHz, MeOD): δ 8.98 |
| | | (d, J = 2.6 Hz, 1H), 8.89 (d, J = 2.4 Hz, |
| | | 1H), 7.99-7.93 (m, 2H), 7.71 (d, J = 8.0 |
| | | Hz, 1H), 7.65 (ddd, J = 8.4, 6.9, 1.5 |
| | | Hz, 1H), 7.56 (ddd, J = 8.1, 6.9, 1.1 |
| | | Hz, 1H), 7.43-7.38 (m, 2H), 6.88-6.82 |
| | | (m, 2H), 4.02 (t, J = 6.1 Hz, 2H), |
| | | 2.72-2.51 (m, 6H), 2.08-1.97 (m, 2H), |
| | | 1.72-1.62 (m, 4H), 1.53 (dd, J = 11.8, 7.2 Hz, 2H). |
| 433 | 485.4 | 1H NMR (500 MHz, DMSO) δ 10.12 (s, 1H), |
| | | 9.04 (s, 1H), 8.26 (s, 1H), 8.23- |
| | | 8.19 (m, 2H), 8.03 (d, J = 8.6 Hz, 1H), |
| | | 7.87-7.84 (m, 1H), 7.79 (d, J = 8.1 Hz, |
| | | 1H), 7.69-7.65 (m, 1H), 7.44-7.33 (m, 3H), |
| | | 6.88 (d, J = 8.5 Hz, 1H), 3.95 (t, |
| | | J = 6.4 Hz, 2H), 3.73 (s, 3H), 2.48-2.43 |
| | | (m, 2H), 2.40 (s, 4H), 1.89-1.82 (m, |
| | | 2H), 1.61-1.42 (m, 4H), 1.41-1.32 (m, 2H). |
| 434 | 560.5 | 1H NMR (400 MHz, dmso) δ 8.09 (d, |
| | | J = 33.4 Hz, 1H), 7.93 (d, J = 6.4 Hz, |
| | | 1H), 7.29-7.23 (m, 3H), 7.16-7.01 (m, 2H), |
| | | 6.95-6.91 (m, 1H), 6.29-6.23 |
| | | (m, 1H), 4.01 (t, J = 5.9 Hz, 2H), |
| | | 3.75-3.72 (m, 4H), 3.66 (s, 3H), 3.60-3.57 |
| | | (m, 4H), 3.24-3.16 (m, 4H), 2.91 (s, 2H), |
| | | 2.10 (dd ,J = 9.9, 5.8 Hz, 2H), 1.93- |
| | | 1.33 (m, 6H). |
| 435 | 489.4 | 1H NMR (400 MHz, dmso) δ 9.46 (s, 1H), |
| | | 8.94 (s, 1H), 8.53 (s, 1H), 8.25 (s, |
| | | 1H), 8.06 (d, J = 8.3 Hz, 1H), 8.01 (d, |
| | | J = 5.7 Hz, 1H), 7.78 (d, J = 2.0 Hz, |
| | | 1H), 7.47 (d, J = 8.3 Hz, 1H), 7.32-7.26 |
| | | (m, 2H), 6.86 (d, J = 8.4 Hz, 1H), |
| | | 6.19 (d, J = 5.7 Hz, 1H), 4.33 (s, 2H), |
| | | 3.94-3.91 (m, 2H), 3.64 (s, 3H), 2.46- |
| | | 2.40 (m, 4H), 1.89-1.82 (m, 2H), 1.75 |
| | | (s, 1H), 1.55-1.48 (m, 4H), 1.43- |
| | | 1.36 (m, 2H). |
| 436 | 474.6 | 1H NMR (500 MHz, CD3CN) δ 8.00 (s, 1H), |
| | | 7.95 (d, J = 5.7 Hz, 2H), 7.69 (s, |
| | | 1H), 7.55 (d, J = 8.6 Hz, 1H), 7.48 (s, |
| | | 1H), 7.33 (d, J = 2.5 Hz, 1H), 7.28- |
| | | 7.24 (m, 1H), 7.15-7.12 (m, 1H), 6.83 (d, |
| | | J = 8.7 Hz, 1H), 6.14 (d, J = 5.8 Hz, |
| | | 1H), 3.98 (t, J = 6.5 Hz, 2H), 3.67 (s, 3H), |
| | | 2.46-2.38 (m, 7H), 1.90-1.87 (m, |
| | | 2H), 1.58-1.52 (m, 4H), 1.46-1.40 (m, 2H). |
| 437 | 488.4 | 1H NMR (400 MHz, dmso) δ 9.27 (s, 1H), |
| | | 8.87 (s, 1H), 8.25 (s, 1H), 7.96- |
| | | 7.93 (m, 1H), 7.86 (s, 1H), |
| | | 7.55 (d, J = 9.0 Hz, 1H), |
| | | 7.42 (d, J = 8.8 Hz, 1H), |
| | | 7.32 (s, 1H), 7.20 (d, J = 8.9 Hz, 1H), 6.81 |
| | | (d, J = 7.9 Hz, 1H), 6.14 (d, J = 4.8 |
| | | Hz, 1H), 4.02 (d, J = 1.0 Hz, 4H), 3.93 |
| | | (t ,J = 6.2 Hz, 3H), 3.60 (s, 4H), 2.40- |
| | | 2.29 (m, 9H), 1.86-1.79 (m, 3H), |
| | | 1.51-1.43 (m, 6H), 1.37 (d, J = 4.5 Hz, |
| | | 3H). |
| 438 | 490.4 | 1H NMR (400 MHz, DMSO) δ 9.39 (s, 1H), |
| | | 8.91 (s, 1H), 8.41 (d, J = 1.5 Hz, |
| | | 1H), 7.98 (d ,J = 5.7 Hz, 1H), 7.87 (d, |
| | | J = 8.7 Hz, 1H), 7.72 (d, J = 5.4 Hz, |
| | | 1H), 7.54-7.47 (m, 1H), 7.34-7.26 (m, 2H), |
| | | 7.24-7.19 (m, 1H), 6.83 (d, J = |
| | | 8.7 Hz, 1H), 6.19 (d, J = 5.7 Hz, 1H), |
| | | 3.94 (t, J = 6.4 Hz, 2H), 3.61 (s, 3H), |
| | | 2.47-2.34 (m, 6H), 1.88-1.82 (m, 2H), |
| | | 1.54-1.47 (m, 4H), 1.38 (d, J = 5.2 |
| | | Hz, 2H). |
| 439 | 517.5 | 1H NMR (500 MHz, DMSO) δ 9.97 (s, 1H), |
| | | 9.12 (s, 1H), 8.20 (s, 1H), 8.10 |
| | | (dd, J = 18.7, 3.7 Hz, 2H), 7.72 (d, |
| | | J = 8.3 Hz, 1H), 7.29-7.23 (m, 2H), 6.90 |
| | | (d, J = 8.6 Hz, 1H), 6.27 (d, J = 5.7 |
| | | Hz, 1H), 3.95 (t, J = 6.5 Hz, 2H), 3.68 (s, |
| | | 3H), 3.02 (s, 3H), 2.42-2.29 (m, 6H), |
| | | 1.87-1.82 (m, 2H), 1.51-1.45 (m, |
| | | 3H), 1.40-1.35 (m, 2H). |
| 440 | 473.5 | 1H NMR (500 MHz, DMSO) δ 10.89 (s, 1H), |
| | | 9.09 (s, 1H), 8.80 (s, 1H), 7.92 |
| | | (d, J = 5.7 Hz, 1H), 7.58 (s, 1H), 7.45 (d, |
| | | J = 8.4 Hz, 1H), 7.39 (d, J = 2.4 Hz, |
| | | 1H), 7.28-7.25 (m, 2H), 7.22-7.19 (m, 1H), |
| | | 6.77 (d, J = 8.8 Hz, 1H), 6.38- |
| | | 6.36 (m, 1H), 6.13 (d, J = 5.8 Hz, 1H), |
| | | 3.89 (t, J = 6.5 Hz, 2H), 3.56 (s, 3H), |
| | | 2.39-2.27 (m, 6H), 1.83-1.77 (m, 2H), |
| | | 1.51-1.46 (m, 4H), 1.39-1.33 (m, |
| | | 2H). |
| 441 | 473.4 | 1H NMR (500 MHz, DMSO) δ 10.97 (s, 1H), |
| | | 9.00 (s, 1H), 8.78 (s, 1H), 7.89 |
| | | (d, J = 5.8 Hz, 2H), 7.36 (d, J = 2.0 Hz, |
| | | 1H), 7.33-7.28 (m, 2H), 7.24-7.22 |
| | | (m, 1H), 7.17-7.13 (m, 1H), 6.78 (d, |
| | | J = 8.7 Hz, 1H), 6.34 (s, 1H), 6.08 (d, J = |
| | | 5.8 Hz, 1H), 3.93-3.89 (m, 2H), 3.58 |
| | | (s, 3H), 2.40-2.24 (m, 6H), 1.85- |
| | | 1.78 (m, 2H), 1.52-1.45 (m, 4H), |
| | | 1.40-1.34 (m, 2H). |
| 442 | 504.6 | 1H NMR (500 MHz, DMSO) δ 9.16 (s, 1H), |
| | | 8.84 (s, 1H), 7.94 (d, J = 5.8 Hz, |
| | | 1H), 7.60 (d, J = 7.4 Hz, 1H), 7.46 (d, |
| | | J = 2.0 Hz, 1H), 7.33 (d, J = 2.2 Hz, |
| | | 1H), 7.25 (dd, J = 8.7, 2.4 Hz, 1H), |
| | | 6.97 (d, J-8.4 Hz, 1H), 6.82 (d, J = 8.8 |
| | | Hz, 1H), 6.14 (d, J = 5.7 Hz, 1H), |
| | | 5.04 (d, J = 6.1 Hz, 1H), 4.57-4.50 (m, |
| | | 1H), 3.92 (t, J = 6.4 Hz, 2H), 3.64 (s, 3H), |
| | | 2.73-2.57 (m, 2H), 2.46-2.29 (m, |
| | | 6H), 1.94-1.78 (m, 4H), 1.72-1.60 |
| | | (m, 2H), 1.53-1.45 (m, 4H), 1.42-1.32 |
| | | (m, 2H). |
| 443 | 491.4 | 1H NMR (500 MHz, DMSO) δ 9.55-9.50 (m, 1H), |
| | | 9.35 (s, 1H), 9.00 (s, 1H), |
| | | 8.58 (s, 1H), 8.05-8.01 (m, 2H), 7.35-7.28 |
| | | (m, 2H), 6.85 (d, J = 8.7 Hz, |
| | | 1H), 6.23 (d, J = 5.7 Hz, 1H), |
| | | 3.96-3.92 (m, 2H), 3.63 (s, 3H), 2.43-2.32 |
| | | (m, 6H), 1.86-1.81 (m, 2H), 1.53-1.47 |
| | | (m, 4H), 1.41-1.35 (m, 2H). |
| 444 | 514.6 | 1H NMR (500 MHz, DMSO) δ 9.44 (s, 1H), |
| | | 8.96 (s, 1H), 8.43 (s, 1H), 8.00 (d, |
| | | J = 5.7 Hz, 1H), 7.74 (d, J = 8.9 Hz, 1H), |
| | | 7.64 (d, J = 8.8 Hz, 1H), 7.57 (dd, J = |
| | | 8.8, 2.0 Hz, 1H), 7.33 (s, 1H), 7.31-7.27 |
| | | (m, 1H), 7.27-7.24 (m, 1H), 7.12 |
| | | (dd, J = 8.9, 2.6 Hz, 1H), 6.85 (d, J = 8.7 Hz, |
| | | 1H), 6.23 (d, J = 5.8 Hz, 1H), |
| | | 3.96 (t, J = 6.3 Hz, 2H), 3.86 (s, 3H), 3.63 |
| | | (s, 3H), 2.58 (s, 4H), 1.92 (t, J = 6.8 |
| | | Hz, 2H), 1.55 (d, J = 4.9 Hz, 4H), 1.42 (s, 2H). |
| 445 | 488.4 | 1H NMR (500 MHz, DMSO) δ 9.49 (s, 1H), |
| | | 9.00 (s, 1H), 8.22 (s, 1H), 8.02 (d, |
| | | J = 5.7 Hz, 1H), 7.91 (d, J = 1.0 Hz, 1H), |
| | | 7.64-7.61 (m, 1H), 7.33 (d, J = 2.2 |
| | | Hz, 1H), 7.22-7.19 (m, 1H), 7.15-7.12 (m, |
| | | 1H), 6.84 (d, J = 8.7 Hz, 1H), |
| | | 6.25 (d, J = 5.7 Hz, 1H), 3.94-3.90 (m, |
| | | 2H), 3.84 (s, 3H), 3.57 (s, 3H), 2.41- |
| | | 2.29 (m, 6H), 1.85-1.80 (m, 2H), 1.52-1.47 |
| | | (m, 4H), 1.38 (d, J = 5.1 Hz, |
| | | 2H). |
| 446 | 474.5 | 1H NMR (500 MHz, DMSO) δ 12.77 (s, |
| | | 1H), 9.39 (s, 1H), 8.96 (s, 1H), 8.00 |
| | | (d, J = 5.7 Hz, 1H), 7.96 (d, J = 1.0 Hz, |
| | | 1H), 7.87 (s, 1H), 7.64 (d, J = 8.7 Hz, |
| | | 1H), 7.38 (d, J = 2.4 Hz, 1H), 7.36-7.33 |
| | | (m, 1H), 7.31-7.28 (m, 1H), 6.82 |
| | | (d, J = 8.8 Hz, 1H), 6.22 (d, |
| | | J = 5.7 Hz, 1H), 3.94-3.89 (m, 2H), 3.59 (s, 3H), |
| | | 2.39-2.27 (m, 6H), 1.84-1.78 (m, 2H), |
| | | 1.51-1.43 (m, 4H), 1.37 (d, J = 5.4 |
| | | Hz, 2H). |
| 447 | 503.4 | 1H NMR (500 MHz, DMSO) δ 9.78 (s, 1H), 9.09 |
| | | (s, 1H), 8.77 (dd, J = 4.1, 1.6 |
| | | Hz, 1H), 8.29 (s, 1H), 8.16 (s, 1H), 8.09 (d, |
| | | J = 5.7 Hz, 1H), 7.88 (d, J = 13.3 |
| | | Hz, 1H), 7.55 (dd, J = 8.4, 4.2 Hz, 1H), |
| | | 7.30 (s, 1H), 7.25 (d, J = 8.4 Hz, 1H), |
| | | 6.89 (d, J = 8.7 Hz, 1H), 6.27 (d, J = 5.7 |
| | | Hz, 1H), 3.97 (t, J = 6.4 Hz, 2H), 3.66 |
| | | (s, 3H), 2.40 (t, J = 7.1 Hz, 2H), 2.34 |
| | | (s, 4H), 1.91-1.80 (m, 2H), 1.49 (dt, J = |
| | | 11.0, 5.5 Hz, 4H), 1.38 (d, J = 5.1 Hz, 2H). |
| 448 | 503.5 | 1H NMR (500 MHz, DMSO) δ 9.98 (s, 1H), |
| | | 9.14 (s, 1H), 8.86 (s, 1H). 7.93 (d, |
| | | J = 5.7 Hz, 1H), 7.56 (s, 1H), 7.35 (d, |
| | | J = 8.0 Hz, 1H), 7.30 (s, 1H), 7.22 (dd, J = |
| | | 8.7, 2.1 Hz, 1H), 6.82 (d, J = 8.7 Hz, 1H), |
| | | 6.79 (d, J = 8.4 Hz, 1H), 6.10 (d, J = |
| | | 5.8 Hz, 1H), 3.93 (t, J = 6.3 Hz, 2H), 3.64 |
| | | (s, 3H), 2.80 (t, J = 7.5 Hz, 2H), |
| | | 2.43 (m, 8H, overlap with DMSO), 1.91-1.81 |
| | | (m, 2H), 1.53 (s, 4H), 1.40 (s, |
| | | 2H). |
| 449 | 502.5 | 1H NMR (500 MHz, DMSO) δ 9.68 (s, 1H), 9.05 |
| | | (s, 1H), 8.07 (d, J = 5.7 Hz, |
| | | 1H), 7.84 (d, J = 1.7 Hz, 1H), 7.78 (d, |
| | | J = 8.7 Hz, 1H), 7.55 (d, J = 6.7 Hz, |
| | | 1H), 7.27 (s, 1H), 7.20 (d, J = 8.3 Hz, 1H), |
| | | 6.88 (d, J = 8.7 Hz, 1H), 6.25 (d, J = |
| | | 5.7 Hz, 1H), 3.95 (t, J = 6.4 Hz, 2H), |
| | | 3.67 (s, 3H), 2.87-2.76 (m, 2H), 2.07- |
| | | 1.97 (m, 2H), 1.92-1.81 (m, 2H), |
| | | 1.57-1.45 (m, 4H), 1.44-1.33 (m, 2H). |
| | | 8 protons overlap with DMSO-d6 |
| 450 | 503.5 | 1H NMR (500 MHz, DMSO) δ 9.98 (s, 1H), |
| | | 9.14 (s, 1H), 8.86 (s, 1H), 7.93 (d, |
| | | J = 5.7 Hz, 1H), 7.56 (s, 1H), 7.35 (d, |
| | | J == 8.0 Hz, 1H), 7.30 (s, 1H), 7.22 (dd, J = |
| | | 8.7, 2.1 Hz, 1H), 6.82 (d, J = 8.7 Hz, 1H), 6.79 |
| | | (d, J = 8.4 Hz, 1H), 6.10 (d, J = |
| | | 5.8 Hz, 1H), 3.93 (t, J = 6.3 Hz, 2H), 3.64 |
| | | (s, 3H), 2.80 (t, J = 7.5 Hz, 2H), |
| | | 2.43 (m, 8H, overlap with DMSO), |
| | | 1.91-1.81 (m, 2H), 1.53 (s, 4H), 1.40 (s, |
| | | 2H). |
| 451 | 503.5 | 1H NMR (500 MHz, DMSO) δ 8.82 (s, 1H), |
| | | 8.77 (s, 1H), 7.87 (d, J = 5.8 Hz, |
| | | 1H), 7.35 (d, J = 0.6 Hz, 1H), 7.23 (dd, |
| | | J = 8.7, 2.4 Hz, 1H), 7.20-7.09 (m, |
| | | 2H), 6.79 (d, J = 8.7 Hz, 1H), 6.54 (d, |
| | | J = 8.7 Hz, 1H), 6.02 (d, J = 5.7 Hz, |
| | | 1H), 3.92 (t, J = 6.4 Hz, 2H), 3.63 (s, 3H), |
| | | 3.17-3.11 (m, 2H), 2.81 (s, 3H), |
| | | 2.66 (t, J = 6.4 Hz, 2H), 2.38 (broad s, |
| | | 6H), 1.94-1.87 (m, 2H), 1.86-1.80 |
| | | (m, 2H), 1.51 (s, 4H), 1.39 (s, 2H). |
| 452 | 514.4 | 1H NMR (400 MHz, DMSO) δ 9.46 (s, 1H), |
| | | 8.99 (s, 1H), 8.48 (s, 1H), 7.98 (d, |
| | | J = 5.7 Hz, 1H), 7.66 (t, J = 8.5 Hz, 2H), |
| | | 7.32 (d, J = 7.0 Hz, 2H), 7.14 (d, J = |
| | | 7.7 Hz, 1H), 6.94 (dd, J = 8.9, 2.3 Hz, 2H), |
| | | 6.83 (d, J = 8.7 Hz, 1H), 6.21 (d, J = |
| | | 5.7 Hz, 1H), 3.88 (t, J = 6.5 Hz, 2H), |
| | | 3.81 (s, 3H), 3.56 (s, 3H), 2.37 (dd, J = |
| | | 16.7, 9.3 Hz, 6H), 1.84-1.76 (m, 2H), |
| | | 1.49-1.42 (m, 4H), 1.34 (d, J = 5.0 |
| | | Hz, 2H). |
| 453 | 514.4 | 1H NMR (400 MHz, dmso) δ 9.50 (s, 1H), |
| | | 8.98 (s, 1H), 8.48 (s, 1H), 8.03- |
| | | 7.96 (m, 2H), 7.50 (d, J = 9.2 Hz, 1H), |
| | | 7.33-7.19 (m, 4H), 6.82 (d, J = 8.6 Hz, |
| | | 1H), 6.76 (d, J = 7.7 Hz, 1H), 6.21 (d, |
| | | J = 5.7 Hz, 1H), 3.93 (d, J = 6.2 Hz, |
| | | 2H), 3.90 (s, 3H), 3.59 (s, 3H), 1.86 (s, 2H), |
| | | 1.48 (s, 4H), 1.36 (s, 2H). * 2H |
| | | overlap with solvent peaks, |
| 454 | 498.4 | 1H NMR (500 MHz, DMSO) δ 9.46 (s, 1H), |
| | | 9.00 (s, 1H), 8.38 (s, 1H), 8.03 (t, |
| | | J = 5.9 Hz, 1H), 7.91 (d, J = 8.2 Hz, 1H), |
| | | 7.71 (d, J = 7.2 Hz, 1H), 7.48-7.43 |
| | | (m, 2H), 7.43-7.37 (m, 1H), 7.29 (d, |
| | | J = 7.5 Hz, 2H), 6.86 (d, J = 8.9 Hz, |
| | | 1H), 6.26 (d, J = 5.7 Hz, 1H), 3.97 |
| | | (t, J = 6.4 Hz, 2H), 3.63 (s, 3H), 2.61 (s, |
| | | 3H), 2.42 (s, 2H), 2.37 (s, 4H), 1.90-1.83 |
| | | (m, 2H), 1.54-1.48 (m, 4H), 1.39 |
| | | (s, 2H). |
| 455 | 485.4 | 1H NMR (400 MHz,cd3cn) δ 8.52 |
| | | (s, 1H), 8.32-8.25 (m, 1H), 8.13-8.08 |
| | | (m, 1H), 7.93 (s, 1H), 7.84-7.78 (m, 1H), |
| | | 7.64 (d, J = 8.9 Hz, 1H), 7.61- |
| | | 7.56 (m, 1H), 7.32 (s, 1H), 7.17 (s, 1H), 6.93 |
| | | (d, J = 8.6 Hz, 1H), 6.30 (s, 1H), |
| | | 4.07 (s, 4H), 3.71 (s, 3H), 2.99-2.95 |
| | | (m, 8H), 2.17-2.08 (m, 4H), 1.80- |
| | | 1.50 (m, 2H). |
| 456 | 499.5 | 1H NMR (500 MHz, DMSO) δ 9.66 (s, 1H), |
| | | 9.02 (s, 1H), 8.55 (s, 1H), 8.05 (d, |
| | | J = 5.7 Hz, 1H), 7.99 (d, J = 7.7 Hz, 1H), |
| | | 7.87-7.76 (m, 2H), 7.38-7.30 (m, |
| | | 2H), 7.26 (d, J = 7.5 Hz, 1H), 6.88 (d, |
| | | J = 8.7 Hz, 1H), 6.28 (d, J = 5.7 Hz, |
| | | 1H), 3.97 (t, J = 6.3 Hz, 2H), 3.64 (s, 3H), |
| | | 2.62 (s, 3H), 2.45 (s, 4H), 1.90 (dt, J = |
| | | 13.4, 6.6 Hz, 2H), 1.57-1.49 (m, 4H), |
| | | 1.40 (s, 2H). * one peak obscured by |
| | | DMSO |
| 457 | 519.6 | 1H NMR (500 MHz, DMSO) δ 9.83 (s, 1H), |
| | | 9.12 (s, 1H), 8.87 (dd, J = 4.1, 1.6 |
| | | Hz, 1H), 8.59 (s, 1H), 8.19 (s, 1H), 8.12 (d, |
| | | J = 3.7 Hz, 1H), 8.11 (s, 1H), 7.60 |
| | | (dd, J = 8.3, 4.1 Hz, 1H), 7.32-7.26 (m, |
| | | 2H), 6.92 (d, J = 8.6 Hz, 1H), 6.30 (d, |
| | | J = 5.7 Hz, 1H), 3.99 (t, J = 6.4 Hz, |
| | | 2H), 3.68 (s, 3H), 2.47-2.42 (m, 2H), |
| | | 2.39 (s, 4H), 1.89 (dt, J = 13.3, 6.5 Hz, 2H), |
| | | 1.53 (dt, J = 10.7, 5.6 Hz, 4H), |
| | | 1.41 (s, 2H). |
| 458 | 485.5 | 1H NMR(500 MHz, DMSO) δ 9.92 (s, 1H), |
| | | 9.05 (s, 2H), 8.79 (s, 1H), 8.52 (s, |
| | | 3H), 8.34 (d, J = 5.6 Hz, 1H), 8.06 (d, |
| | | J = 5.7 Hz, 1H), 7.89-7.82 (m, 2H), |
| | | 7.71 (d, J = 5.7 Hz, 1H), 7.33 (s, 1H), |
| | | 7.27-7.24 (m,1H), 6.88 (d, J = 8.7 Hz, |
| | | 1H), 6.31 (d, J = 5.7 Hz, 1H), 3.98-3.95 |
| | | (m, 2H), 3.65 (s, 3H), 2.41-2.37 |
| | | (m, 2H), 2.32 (s, 3H), 1.86-1.81 (m, 2H), |
| | | 1.51-1.45 (m, 4H), 1.37 (s, 2H). |
| 459 | 433.6 | 1H NMR (500 MHz, DMSO): δ 10.51 (s, 1H), |
| | | 9.87 (s, 1H), 8.94 (s, 1H), 8.82 |
| | | (s, 1H), 8.07 (d, J = 6.4 Hz, 1H), 7.97 (d, |
| | | J = 8.7 Hz, 1H), 7.74 (s, 2H), 7.69- |
| | | 7.62 (m, 1H), 7.58 (t, J = 7.0 Hz, 1H), 7.52 (d, |
| | | J = 6.5 Hz, 1H), 7.15 (d, J = 8.7 |
| | | Hz, 1H), 6.46 (d, J = 6.4 Hz, 1H), |
| | | 3.81-3.71 (m, 4H), 3.00-2.93 (m, 4H). |
| 460 | 413.4 | 1H NMR (500 MHz, DMSO): δ 9.88 (s, 1H), |
| | | 9.39 (s, 1H), 8.99 (s, 1H), 8.95 |
| | | (d, J = 2.6 Hz, 1H), 8.13 (d, J = 5.7 Hz, 1H), |
| | | 7.94 (d, J = 8.1 Hz, 1H), 7.87 (d, J = |
| | | 7.9 Hz, 1H), 7.75 (d, J = 8.8 Hz, 2H), |
| | | 7.65-7.53 (m, 2H), 7.31-7.25 (m, |
| | | 2H), 6.37 (d, J = 5.7 Hz, 1H), 4.21 (s, 2H), |
| | | 3.99 (dd, J = 5.8, 4.3 Hz, 2H), 3.72 |
| | | (dd, J = 5.7, 4.4 Hz, 2H). |
| 461 | 463.6, | 1HNMR(500 MHz, CD3CN) δ 8.98 (d, |
| | 465.7 | J = 2.6 Hz, 1H), 8.92 (d, J = 2.5 Hz, |
| | | 1H), 8.20 (s, 1H), 8.09 (d, J = 5.7 Hz, 1H), |
| | | 7.72-7.70 (m, 1H), 7.68 (s, 2H), |
| | | 7.49-7.45 (m, 1H), 7.26 (d, J = 10.0 Hz, 1H), |
| | | 7.17 (dd, J = 8.5, 2.3 Hz, 1H), |
| | | 6.88 (d, J = 8.4 Hz, 1H), 6.29 (d, J = 5.7 |
| | | Hz, 1H), 3.81-3.74 (m, 4H), 3.70 (s, |
| | | 3H), 3.01-2.99 (m, 2H), 2.09-2.06 (m, 2H). |
| 462 | 463.5, | 1HNMR(400 MHz, DMSO) δ 9.97 (s, |
| | 465.6 | 1H), 9.13 (s, 1H), 9.06 (s, 1H), 8.92- |
| | | 8.90 (m,J = 2.4 Hz, 1H), 8.29 (s, 1H), 8.11 (d, |
| | | J = 5.7 Hz, 1H), 7.94 (d, J = 8.9 |
| | | Hz, 1H), 7.80 (s, 1H), 7.59-7.56 (m, 1H), |
| | | 7.27 (s, 1H), 6.89-6.84 (m, 1H), |
| | | 6.32 (d, J = 5.7 Hz, 1H), 3.76-3.72 |
| | | (m, 4H), 3.68 (s, 3H), 3.01-2.95 (m, |
| | | 4H) |
| 463 | 472.5 | 1HNMR(500 MHz, DMSO) δ 9.60 (s, 1H), |
| | | 8.72 (s, 2H), 7.97 (d, J = 5.7 Hz, |
| | | 1H), 7.89 (s, 1H), 7.52-7.44 (m, 2H), |
| | | 7.29 (d, J = 2.4 Hz, 1H), 7.15-7.11 |
| | | (m, 1H), 6.67 (d, J = 2.5 Hz, 1H), 6.50-6.47 |
| | | (m, 1H), 6.18 (d, J = 5.7 Hz, |
| | | 1H), 3.88 (s, 3H), 3.75 (s, 3H), 3.21-3.15 |
| | | (m, 4H), 2.52-2.51 (m, 4H), 2.26 |
| | | (s, 3H). |
| 464 | 476.6, | 1HNMR(500 MHz, DMSO) δ 9.83 (s, 1H), |
| | 478.6 | 8.86 (s, 1H), 8.83 (s, 1H), 8.04- |
| | | 8.01 (m, 2H), 7.94-7.93 (m, 1H), 7.61 (s, 2H), |
| | | 7.50-7.41 (m, 3H), 6.68 (d, J = |
| | | 2.5 Hz, 1H), 6.54-6.51 (m, 1H), 6.22 (d, |
| | | J = 5.6 Hz, 1H), 3.75 (s, 3H), 3.23- |
| | | 3.18 (m, 4H), 2.27 (s, 3H), 1.29-1.22 (m, 2H). |
| 465 | 470.1 | 1H NMR (400 MHz, DMSO) δ 9.78 (s, |
| | | J = 5.1 Hz, 1H), 9.03 (s, 1H), 9.00- |
| | | 8.91 (m, 1H), 8.88-8.83 (m, 1H), 8.08-7.99 |
| | | (m, 1H), 7.90 (d, J = 8.1 Hz, |
| | | 1H), 7.77-7.66 (m, 1H), 7.61-7.44 (m, 4H), |
| | | 6.92 (d, J = 9.0 Hz, 2H), 6.26 |
| | | (d, J = 5.6 Hz, 1H), 4.95 (d, J = 6.9 Hz, 1H), |
| | | 4.48 (p, J = 6.5 Hz, 1H), 3.79- |
| | | 3.51 (m, 4H), 3.15-2.98 (m, 4H), |
| | | 1.21 (d, J = 6.5 Hz, 3H). |
| 466 | 520.4, | 1HNMR(500 MHz, DMSO) δ 9.87 (s, 1H), |
| | 522.2 | 8.93 (s, 1H), 8.84-8.81 (m, 1H), |
| | | 8.14 (s, 1H), 8.02 (d, J = 5.6 Hz, 1H), |
| | | 7.97 (s, 1H), 7.84 (d, J = 8.9 Hz, 1H), |
| | | 7.67-7.65 (m, 1H), 7.59-7.57 (m, 1H), 6.69 |
| | | (d, J = 2.5 Hz, 1H), 6.55-6.52 |
| | | (m, 1H), 6.25 (d, J = 5.7 Hz, 1H), 3.79 |
| | | (s, 3H), 3.24 (s, 4H), 2.66 (s, 4H), 2.36 |
| | | (s, 3H). |
| 467 | 476.5, | 1HNMR(500 MHz, DMSO) δ 9.89 (s, 1H), |
| | 478.4 | 8.89 (s, 2H), 8.06 (s, 1H), 8.02 (d, |
| | | J = 5.6 Hz, 1H), 7.74-7.71 (m, 1H), |
| | | 7.51 (s, 1H), 7.45-7.39 (m, 2H), 6.69 |
| | | (d, J = 2.5 Hz, 1H), 6.55-6.52 (m, 1H), |
| | | 6.24 (d, J = 5.6 Hz, 1H), 3.75 (s, 3H), |
| | | 3.23-3.18 (m, 4H), 2.54-2.51 |
| | | (m, 4H), 2.27 (s, 3H). |
| 468 | 479.4 | 1H NMR (500 MHz, DMSO) δ 9.96 (s, 1H), |
| | | 9.14 (s, 1H), 9.02 (s, 1H), 8.90 (s, |
| | | 1H), 8.18 (d, J = 2.1 Hz, 1H), 8.10 (d, |
| | | J = 5.7 Hz, 1H), 7.95-7.91 (m, 1H), |
| | | 7.74 (s, 1H), 7.50 (s, 1H), 6.32 (d, J = 5.7 Hz, |
| | | 1H), 3.67 (s, 3H), 3.27 (s, 4H), |
| | | 2.46 (s, 4H), 2.22 (s, 3H). |
| 469 | 466.4 | 1HNMR(500 MHz, DMSO) δ 9.96 (s, 1H), |
| | | 9.17 (s, 1H), 9.02 (s, 1H), 8.91 (s, |
| | | 1H), 8.21 (d, J = 2.1 Hz, 1H), 8.11 (d, |
| | | J = 5.7 Hz, 1H), 7.95-7.91 (m, 1H), |
| | | 7.75 (s, 1H), 7.53 (s, 1H), 6.33 (d, |
| | | J = 5.7 Hz, 1H), 3.76-3.72 (m, 4H), 3.70 |
| | | (s, 3H), 3.27-3.24 (m, 4H). |
| 470 | 506.5 | 1HNMR(500 MHz, DMSO) δ 9.92 (s, 1H), |
| | | 9.07 (d, J = 12.0 Hz, 2H), 8.92 (s, |
| | | 1H), 8.10 (d, J = 5.7 Hz, 1H), 7.93 (t, |
| | | J = 9.8 Hz, 1H), 7.64 (s, 1H), 7.25 (s, |
| | | 1H), 7.20 (d, J = 7.8 Hz, 1H), 6.80 (d, |
| | | J = 8.5 Hz, 1H), 6.30 (d, J = 5.7 Hz, |
| | | 1H), 3.66 (s, 3H), 2.94 (s, 2H), 2.68 (s, 2H), |
| | | 2.59-2.55 (m, 2H), 2.17 (s, 3H), |
| | | 1.06 (s, 6H). |
| 470 | 478.3 | 1H NMR (500 MHz, DMSO) δ 9.92 (s, 1H), |
| | | 9.00 (s, 2H), 8.91 (s, 1H), 8.09 (d, |
| | | J = 5.7 Hz, 1H), 8.05 (d, J = 2.0 Hz, 1H), |
| | | 7.93 (dd, J = 11.3, 8.2 Hz, 1H), 7.71 |
| | | (s, 1H), 7.41 (s, 1H), 6.30 (d, J = 5.7 Hz, 1H), |
| | | 4.84 (s, 1H), 4.56 (s, 1H), 3.86 |
| | | (d, J = 7.3 Hz, 1H), 3.77 (dd, J = 7.3, 1.6 |
| | | Hz, 1H), 3.65 (d, J = 6.2 Hz, 4H), |
| | | 3.37 (d, J = 10.5 Hz, 1H), 1.86 (dd, |
| | | J = 9.5, 1.9 Hz, 1H), 1.79 (d, J = 9.5 Hz, |
| | | 1H). |
| 471 | 479.4 | 1H NMR (500 MHz, DMSO) δ 10.02 (s, 1H), |
| | | 9.21 (s, 1H), 9.03-8.93 (m, |
| | | 2H), 8.22 (s, 1H), 8.13 (d, J = 5.7 Hz, 1H), |
| | | 7.99-7.91 (m, 1H), 7.86 (s, 1H), |
| | | 7.77 (s, 1H), 7.63 (s, 1H), 6.36 (d, |
| | | J = 5.7 Hz, 1H), 3.81 (s, 2H), 3.72 (s, 3H), |
| | | 3.51 (t, J = 5,2 Hz, 2H), |
| | | 3.27 (t, J = 5.1 Hz, 2H). |
| 472 | 479.4 | 1H NMR (500 MHz, DMSO) δ 9.82 (s, 1H), |
| | | 8.82 (broad s, 1H), 8.77 (s, 1H), |
| | | 8.17 (s, 1H), 8.00 (d, J = 5.6 Hz, 1H), 7.88 |
| | | (dd, J = 11.4, 8.1 Hz, 1H), 7.61 (s, |
| | | 1H), 7.46 (broad s, 1H), 6.40 (d, J = 8.4 Hz, 1H), |
| | | 6.21 (d, J = 5.6 Hz, 1H), 3.79 |
| | | (s, 3H), 3.51 (m, 4H), 2.48-2.43 |
| | | (m, 4H), 2.25 (s, 3H). |
| 473 | 479.5 | 1H NMR (400 MHz, dmso) δ 9.81 (s, 1H), |
| | | 8.85 (s, 1H), 8.70 (s, 1H), 8.16 (s, |
| | | 1H), 8.11 (s, 1H), 8.05 (s, 1H), 7.96 (d, J = 5.6 Hz, |
| | | 1H), 7.83 (dd, J = 11.7, 8.0 |
| | | Hz, 1H), 7.51 (s, 1H), 6.46 (s, 1H), 6.18 (d, |
| | | J = 5.7 Hz, 1H), 3.74 (s, 3H), 3.55- |
| | | 3.49 (m, 4H), 2.44-2.38 (m, 4H), 2.20 (s, 3H). |
| | | 1 H overlaps with water |
| 474 | 547.4 | 1H NMR (500 MHz, DMSO) δ 9,99 (s, 1H), |
| | | 9.16 (s, 1H), 9.03 (s, 1H), 8.93 (§, |
| | | 1H), 8.21 (d, J = 2.1 Hz, 1H), 8.12 (d, |
| | | J = 5.7 Hz, 1H), 7.94 (dd, J = 11,5, 7.9 |
| | | Hz, 1H), 7.81-7.69 (m, 1H), 7.53 (s, 1H), |
| | | 6.34 (d, J = 5.7 Hz, 1H), 3.69 (s, |
| | | 3H), 3.31-3.20 (m, 6H), 2.81-2.75 (m, 4H). |
| 475 | 547.4 | 1H NMR (500 MHz, DMSO) δ 10.01 (s, 1H), |
| | | 9.22 (s, 1H), 9.01 (s, 1H), 8.92 |
| | | (s, 1H), 8.22 (t, J = 5.1 Hz, 1H), 8.11 (d, |
| | | J = 5.7 Hz, 1H), 7.96-7.91 (m, 1H), |
| | | 7.73 (s, 1H), 7.55 (s, 1H), 6.33 (d, J = 5.7 Hz, |
| | | 1H), 3.68 (s, 3H), 3.61 (d, J = |
| | | 10.5 Hz, 1H), 3.24-3.16 (m, 3H), 3.10 (d, |
| | | J = 9.0 Hz, 1H), 2.94 (d, J = 13.0 |
| | | Hz, 1H), 2.57-2.52 (m, 1H), 2.44 (s, 3H). |
| 476 | 492.7 | 1H NMR (500 MHz, DMSO) δ 9.98 (s, 1H), |
| | | 9.14 (s, 1H), 9.02 (s, 1H), 8.89 (s, |
| | | 1H), 8.16 (d, J = 2.0 Hz, 1H), 8.11 (d, |
| | | J = 5.7 Hz, 1H), 7.93 (d, J = 8.9 Hz, |
| | | 1H), 7.84 (s, 1H), 7.58 (dd, J = 8.9, 2.4 Hz, 1H), |
| | | 7.51 (s, 1H), 6.33 (d, J = 5.7 |
| | | Hz, 1H), 4.20 (s, 1H), 3.68 (s, 3H), |
| | | 3.43-3.34 (m, J = 12.4 Hz, 2H), 3.25- |
| | | 3.14 (m, J = 7.9 Hz, 2H), 1.66-1.51 |
| | | (m, 4H), 1.16 (s, 3H). |
| 477 | 477.5, | 1HNMR(500 MHz, DMSO) δ 10.00 (s, 1H), |
| | 479.9 | 9.17 (s, 1H), 9.02 (s, 1H), 8.89 (s, |
| | | 1H), 8.20 (d, J = 2.0 Hz, 1H), 8.11 (d, |
| | | J = 5.7 Hz, 1H), 7.93 (d, J = 8.9 Hz, |
| | | 1H), 7.84 (s, 1H), 7.59-7.51 (m, 2H), 6.34 |
| | | (d, J = 5.7 Hz, 1H), 3.68 (s, 3H), |
| | | 3.27 (s, 4H), 2.45 (s, 4H), 2.22 (s, 3H). |
| 478 | 465.3 | 1H NMR (500 MHz, DMSO): δ 9.96 (s, 1H), |
| | | 9.13 (s, 1H), 9.01 (s, 1H), 8.91 |
| | | (s, 1H), 8.18 (s, 1H), 8.10 (d, J = 5.7 |
| | | Hz, 1H), 7.97-7.90 (m, 1H), 7.75 (s, |
| | | 1H), 7.51 (s, 1H), 6.32 (d, J = 5.6 Hz, |
| | | 1H), 3.67 (s, 3H), 3.25-3.19 (m, 4H), |
| | | 2.95-2.83 (m, 4H), 2.04 (s, 1H). |
| 479 | 502.7 | 1H NMR (500 MHz, DMSO): δ 10.02 (s, 1H), |
| | | 9.20 (s, 1H), 9.02 (s, 1H), 8.89 |
| | | (s, 1H), 8.21 (d, J = 2.0 Hz, 1H), 8.12 (d, |
| | | J = 5.7 Hz, 1H), 7.93 (d, J = 8.9 Hz, |
| | | 1H), 7.83 (s, 1H), 7.58 (dd, J = 8.9, 2.4 Hz, |
| | | 1H), 7.55 (s, 1H), 6.35 (d, J = 5.7 |
| | | Hz, 1H), 3.80 (s, 2H), 3.70 |
| | | (s, 3H), 2.69-2.60 (m, 4H). |
| | | 4H overlap with water |
| | | peak |
| 480 | 505.7, | 1HNMR(500 MHz, DMSO) δ 9.98 (s, 1H), |
| | 507.8 | 9.15 (s, 1H), 9.03 (s, 1H), 8.89 (s, |
| | | 1H), 8.19 (d, J = 2.1 Hz, 1H), 8.11 (d, |
| | | J = 5.7 Hz, 1H), 7.93 (d, J = 8.9 Hz, |
| | | 1H), 7.85 (s, 1H), 7.59-7.56 (m, 1H), 7.53 |
| | | (s, 1H), 6.34 (d, J = 5.7 Hz, 1H), |
| | | 3.69 (s, 3H), 3.25 (s, 2H), 2.98 (s, 2H), |
| | | 2.59-2.55 (m, 2H), 2.16 (s, 3H), |
| | | 1.03 (s, 6H). |
| 481 | 519.7 | 1H NMR (500 MHz, DMSO): δ 9.98 (s, 1H), |
| | | 9.17 (s, 1H), 9.01 (s, 1H), 8.89 |
| | | (s, 1H), 8.21 (d, J = 2.1 Hz, 1H), 8.11 (d, |
| | | J = 5.7 Hz, 1H), 7.93 (d, J = 8.9 Hz, |
| | | 1H), 7.85 (s, 1H), 7.58 (dd, J = 8.9, 2.4 |
| | | Hz, 1H), 7.54 (s, 1H), 6.34 (d, J = 5.7 |
| | | Hz, 1H), 4.56 (t, J = 6.5 Hz, 2H), 4.48 (t, |
| | | J = 6.1 Hz, 2H), 3.68 (s, 3H), 3.53- |
| | | 3.41 (m, 1H), 2.45-2.37 (m, 4H). |
| | | 4H overlap with water peak. |
| 482 | 515.5 | 1H NMR (500 MHz, DMSO) δ 9.97 (s, 1H), |
| | | 9.16 (s, 1H), 8.99 (s, 1H), 8.92 (s, |
| | | 1H), 8.23 (s, 1H), 8.20 (d, J = 2.1 Hz, 1H), |
| | | 8.11 (d, J = 5.7 Hz, 1H), 7.95- |
| | | 7.91 (m, 1H), 7.73 (s, 1H), 7.55 (s, 1H), |
| | | 6.33 (d, 1H), 3.66 (s, 3H), 3.64-3.59 |
| | | (m, 2H), 3.16-3.06 (m, 2H), 2.99-2.95 |
| | | (m, 1H), 2.87-2.73 (m, 3H). |
| 483 | 513.8, | 1H NMR (500 MHz, MeOD) δ 9.05 (s, 2H), |
| | 515.5 | 8.79 (d, J = 2.4 Hz, 2H), 8.10- |
| | | 8.07 (m, 4H), 7.92 (d, J = 8.9 Hz, 2H), |
| | | 7.74 (s, 2H), 7.65 (s, 2H), 7.59 (d, J = |
| | | 2.3 Hz, 1H), 7.57 (d, J = 2.3 Hz, 1H), |
| | | 6.34 (d, J = 5.8 Hz, 2H), 6.06-5.81 (m, |
| | | 2H), 3.73 (d, J = 12.5 Hz, 10H), |
| | | 3.30-2.93 (m, 12H). |
| 484 | 507.4 | 1H NMR (500 MHz, DMSO) δ 9.95 (s, 1H), |
| | | 9.12 (s, 1H), 9.03 (s, 1H), 8.89 (s, |
| | | 1H), 8.16 (d, J = 2.1 Hz, 1H), 8.10 (d, |
| | | J = 5.7 Hz, 1H), 7.95-7.91 (m, 1H), |
| | | 7.73 (s, 1H), 7.49 (s, 1H), 6.32 (d, J = 5.7 Hz, |
| | | 1H), 3.67 (s, 3H), 3.24 (s, 2H), |
| | | 2.99 (s, 2H), 2.59-2.56 (m, 2H), |
| | | 2.17 (s, 3H), 1.04 (s, 6H). |
| 485 | 477.4 | 1H NMR (500 MHz, DMSO) δ 9.85 (s, 1H), |
| | | 8.87 (s, 1H), 8.79 (s, 1H), 8.15 (s, |
| | | 1H), 8.00 (d, J = 5.6 Hz, 1H), 7.90 |
| | | (d, J = 8.9 Hz, 1H), 7.68 (doublet |
| | | overlapping with a broad singlet, J = 8.1 Hz, |
| | | 2H), 7.54 (dd, J = 8.9, 2.3 Hz, |
| | | 1H), 6.39 (d, J = 8.4 Hz, 1H), 6.23 (d, |
| | | J = 5.6 Hz, 1H), 3.81 (s, 3H), 3.52 (s, |
| | | 4H), 2.47 (s, 4H), 2.25 (s, 3H). |
| 486 | 477.4 | 1H NMR (500 MHz, DMSO) δ 9.87 (s, 1H), |
| | | 8.91 (s, 1H), 8.77 (s, 1H), 8.20 (s, |
| | | 1H), 8.10 (s, 1H), 8.01 (d, J = 5.3 Hz, 1H), |
| | | 7.90 (d, J = 8.9 Hz, 1H), 7.76 (8, |
| | | 1H), 7.55 (dd, J = 8.9, 2.3 Hz, 1H), 6.50 |
| | | (s, 1H), 6.25 (d, J = 5.6 Hz, 1H). 3.80 |
| | | (s, 3H), 3.57 (s, 4H), 2.48-2.42 |
| | | (m, 4H), 2.24 (s, 3H). |
| 487 | 494.4 | 1H NMR (500 MHz, DMSO) δ 9.84 (s, 1H), |
| | | 8.90-8.71 (m, 2H), 8.14 (s, 1H), |
| | | 8.00 (d, J = 5.5 Hz, 1H), 7.88 (dd, |
| | | J = 11,4, 8.1 Hz, 1H), 7.63-7.39 (m, 2H), |
| | | 6.39 (d, J = 8.4 Hz, 1H), 6.21 (d, |
| | | J = 5.6 Hz, 1H), 4.35 (s, 1H), 3.87-3.75 (m), |
| | | 5H), 3.43-3.36 (m, 2H), 1.61-1.50 |
| | | (m, 4H), 1.17 (s, 3H). |
| 488 | 494.4 | 1H NMR (500 MHz, DMSO) δ 9.86 (s, 1H), |
| | | 8.90 (s, 1H), 8.75 (s, 1H), 8.12 (s, |
| | | 1H), 8.06 (s, 1H), 8.00 (d, J = 5.6 Hz, 1H), |
| | | 7.87 (dd, J = 11.4, 8.1 Hz, 1H), |
| | | 7.60 (s, 1H), 6.47 (s, 1H), 6.22 (d, J = 5.6 Hz, |
| | | 1H), 4.34 (s, 1H), 3.90 (d, J = |
| | | 12.9 Hz, 2H), 3.77 (s, 3H), 3.46-3.38 |
| | | (m, 2H), 1.61-1.48 (m, 4H), 1.17 (s, |
| | | 3H). |
| 489 | 492.6 | 1H NMR (500 MHz, DMSO) δ 9.87 (s, 1H), |
| | | 8.79 (s, 1H), 8.11 (s, 1H), 8.00 (d, |
| | | J = 5.5 Hz, 1H), 7.90 (d, J = 8.9 Hz, 1H), |
| | | 7.63 (d, J = 8.2 Hz, 1H), 7.54 (dd, J = |
| | | 8.9, 2.3 Hz, 1H), 6.40 (d, J = 8.4 Hz, 1H), |
| | | 6.23 (d, J = 5.6 Hz, 1H), 4.34 (s, |
| | | 1H), 3.88-3.73 (m, 5H), 3.49-3.37 |
| | | (m, 2H), 1.61-1.47 (m, 4H), 1.16 (s, |
| | | 3H). |
| 490 | 492.6 | 1H NMR (500 MHz, DMSO) δ 9.80 (s, 1H), |
| | | 8.70 (s, 1H), 8.07 (s, 1H), 8.00 (s, |
| | | 1H), 7.93 (d, J = 5.4 Hz, 1H), 7.82 (d, |
| | | J = 8.9 Hz, 1H), 7.67 (s, 1H), 7.47 (dd, J = |
| | | 8.9. 2.3 Hz, 1H). 6.40 (s, 1H), 6.16 (d, |
| | | J = 5.6 Hz, 1H), 4.25 (s, 1H), 3.87- |
| | | 3.78 (m, 2H), 3.71 (s, 3H), 3.41-3.31 |
| | | (m, 2H), 1.53-1.39 (m, 4H), 1.09 (s, |
| | | 3H) |
| 491 | 519.5 | 1H NMR (500 MHz, DMSO) δ 12.51 (s, 1H), |
| | | 9.71 (s, 1H), 9.23 (s, 1H), 8.83 |
| | | (s, 1H), 8.30-8.09 (m, 3H), 8.03 (d, |
| | | J = 8.1 Hz, 1H), 7.89 (s, 1H), 7.76-7.60 |
| | | (m, 2H), 7.42 (s, 1H), 4.28 (s, 1H), |
| | | 3.71 (s, 3H), 3.48-3.41 (m, 2H), 3.26- |
| | | 3.21 (m, 2H), 1.71-1.57 (m, 4H), 1.20 (s, 3H). |
| 492 | 519.4 | 1H NMR (500 MHz, DMSO) δ 12.43 (s, 1H), |
| | | 8.80 (s, 1H), 8.26 (s, 1H), 8.20 |
| | | (s, 1H), 8.12 (s, 1H), 8.07 (s, 1H), 7.99 |
| | | (d, J = 8.5 Hz, 1H), 7.75-7.52 (m, |
| | | 3H), 6.50 (s, 1H), 4.41 (s, 1H), 3.98-3.87 |
| | | (m, 2H), 3.76 (s, 3H), 3.48-3.39 |
| | | (m, 2H), 1.67-1.51 (m, 4H), 1.21 (s, 3H). |
| 493 | 529.6 | 1H NMR (500 MHz, DMSO) δ 12.43 (d, |
| | | J = 2.0 Hz, 1H), 9.46-9.33 (m, 1H), |
| | | 8.79 (s, 1H), 8.51 (s, 1H), 8.31 (s, 1H), 8.20 |
| | | (s, 1H), 8.12 (d, J = 4.4 Hz, 2H), |
| | | 7.99 (d, J = 8.5 Hz, 1H), 7.70-7.61 (m, 2H), |
| | | 6.56 (s, 1H), 3.86 (s, 2H), 3.78 |
| | | (s, 3H), 3.65 (s, 4H), 2.70-2.61 (m, 4H). |
| 494 | 519.6 | 1H NMR (500 MHz, DMSO) δ 9.87 (s, 1H), |
| | | 8.78 (s, 1H), 8.21 (s, 1H), 8.10 (s, |
| | | 1H), 8.01 (d, J = 5.5 Hz, 1H), 7.90 (d, |
| | | J = 8.9 Hz, 1H), 7.77 (s, 1H), 7.55 (dd, J = |
| | | 8.9, 2.3 Hz, 1H), 6.51 (s, 1H), 6.25 (d, |
| | | J = 5.6 Hz, 1H), 4.59 (dd, J = 6.5 Hz, |
| | | 2H), 4.50 (dd, J = 6.1 Hz, 2H), 3.81 (s, 3H), |
| | | 3.61 (s, 4H), 3.45 (dddd, J = 6.3 |
| | | Hz, 1H), 2.44-2.34 (m, 4H). |
| 495 | 546.5 | 1H NMR (500 MHz, DMSO) δ 12.42 (s, 1H), |
| | | 8.79 (s, 1H), 8.29 (s, 1H), 8.20 |
| | | (s, 1H). 8.15-8.08 (m, 2H), 7,99 (d, |
| | | J = 8.4 Hz, 1H), 7.74-7.57 (m, 3H), |
| | | 6.53 (s, 1H), 4.61 (dd, J = 6.5 Hz, 2H), 4.53 |
| | | (dd, J = 6.1 Hz, 2H), 3.77 (s, 3H), |
| | | 3.65-3.59 (m, 4H), 3.53-3.47 (m, 1H), |
| | | 2.46-2.41 (m, 4H). |
| 496 | 489.6 | 1H NMR (500 MHz, DMSO) δ 9.90 (s, 1H), |
| | | 8.79 (s, 1H), 8.14 (s, 1H), 8.07 (s, |
| | | 1H), 8.00 (s, 1H), 7.90 (d, J = 9.1 Hz, 1H), |
| | | 7.74 (br, 1H), 7.54 (d, J = 9.1 Hz, |
| | | 1H), 6.42 (s, 1H), 6.25 (d, J = 4.6 Hz, |
| | | 1H), 3.79 (s, 3H), 3.62-3.50 (m, 2H), |
| | | 3.46-3.37 (m, 2H), 2.94-2.83 |
| | | (m, 2H), 0.56-0.43 (m, 4H). |
| 497 | 479.5 | 1H NMR (500 MHz, DMSO) δ 9.94 (s, 1H), |
| | | 9.13 (s, 2H), 9.00 (d, J = 2.4 Hz, |
| | | 1H), 8.10 (d, J = 5.7 Hz, 1H), 8.06 (d, |
| | | J = 8.7 Hz, 2H), 7.70 (s, 1H), 7.41 (s, |
| | | 1H), 7.23 (s, 1H), 7.08 (s, 1H), 6.78 (d, |
| | | J = 8.7 Hz, 1H), 6.32 (d, J = 5.7 Hz, |
| | | 1H), 4.30-4.22 (m, 3H), |
| | | 3.94-3.88 (m, 2H), 2.23 (s, 6H). |
| 498 | 477.4 | 1H NMR (500 MHz, DMSO) δ 9.96 (s, 1H), |
| | | 9.31 (s, 1H), 9.02 (d, J = 2.3 Hz, |
| | | 1H), 8.84 (s, 1H), 8.40 (d, J = 2.3 Hz, 1H), |
| | | 8.13 (d, J = 5.7 Hz, 1H), 8.02- |
| | | 7.89 (m, 2H), 7.83-7.72 (m, 1H), 6.35 (d, |
| | | J = 5.7 Hz, 1H), 3.99 (t, J = 8.5 Hz, |
| | | 2H), 3.80 (s, 2H), 3.00 |
| | | (t, J = 8.3 Hz, 2H), 2.30 (s, 6H). |
| 499 | 466.2 | 1H NMR (500 MHz, DMSO) δ 9.27 (s, 1H), 8.94 |
| | | (s, 2H), 8.12 (dd, J = 5.7, 3.6 |
| | | Hz, 1H), 8.01 (d, J = 2.2 Hz, 1H), 7.99 |
| | | (d, J = 2.3 Hz, 1H), 7.93 (s, 1H), 7.85 |
| | | (s, 1H), 7.76 (s, 1H), 6.35 (d, J = 5.7 Hz, |
| | | 1H), 4.48-4.40 (m, 1H), 4.30 (dd, J = |
| | | 11.5, 2.2 Hz, 1H), 4.01-3.93 (m, J = 11.4, |
| | | 4.5 Hz, 1H), 2.59-2.53 (m, 2H), |
| | | 2.25 (s, 6H). |
| 500 | 464.4 | 1H NMR (500 MHz, DMSO) δ 10.09 (s, 1H), |
| | | 9.30 (s, 1H), 8.93 (s, 1H), 8.38 |
| | | (s, 1H), 8.13 (s, 1H), 8.12 (d, J = 5.7 Hz, |
| | | 1H), 7.97 (d, J = 2.3 Hz, 1H), 7.94 (d, |
| | | J = 8.9 Hz, 1H), 7.90 (s, 1H), 7.80 (s, |
| | | 1H), 7.59 (dd, J = 8.9, 2.4 Hz, 1H), 6.37 |
| | | (d, J = 5.7 Hz, 1H), 4.43 (dd, J = 11.6, |
| | | 2.2 Hz, 1H), 4.33 (td, J = 8.4, 6.5 Hz, |
| | | 1H), 4.11 (dd, J = 11.7, 7.3 Hz, 1H), |
| | | 2.83-2.80 (m, 1H), 2.48-2.41 (m, 1H), |
| | | 2.17 (s, 6H). |
| 501 | 451.2 | 1H NMR (400 MHz, dmso) δ 9.80 (s, 1H), |
| | | 9.16 (s, 1H), 8.95-8.79 (m, 2H), |
| | | 8.08 (d, J = 5.7 Hz, 1H), 7.87 (d, J = 8.3 Hz, |
| | | 1H), 7.79 (s, 1H), 7.53 (t, J = 8.2 |
| | | Hz, 1H), 7.47-7.37 (m, 2H), 7.28-7.16 |
| | | (m, 2H), 6.39 (d, J = 2.9 Hz, 1H), |
| | | 6.27 (d, J = 5.7 Hz, 1H), 3.83-3.56 (m, |
| | | 4H), 3.08-2.84 (m, 2H), 1.93-1.74 |
| | | (m, 1H), 1.34-0.97 (m, 4H). |
| 502 | 435.3 | 1H NMR (400 MHz, DMSO) δ 9.83 (s, 1H), |
| | | 9.18 (s, 1H), 8.93 (d, J = 2.4 Hz, |
| | | 1H), 8.90-8.79 (m, 1H), 8.08 (d, J = 5.6 Hz, |
| | | 1H), 7.94-7.79 (m, 2H), 7.54 (t, |
| | | J = 8.3 Hz, 1H), 7.48-7.34 (m, 3H), 7.24 (d, |
| | | J = 3.1 Hz, 2H), 6.36 (d, J = 2.9 |
| | | Hz, 1H), 6.27 (d, J = 5.7 Hz, 1H), |
| | | 3.82-3.66 (m, 2H), 2.28-2.13 (m, 1H), |
| | | 1.52-1.27 (m, 6H), 1.08-0.94 (m, 2H). |
| 503 | 449.4 | 1H NMR (400 MHz, dmso) δ 9.97-9.85 |
| | | (m, 1H), 9.34-9.18 (m, 1H), 8.95- |
| | | 8.78 (m, 2H), 8.06 (d, J = 5.7 Hz, 1H), |
| | | 7.87 (d, J = 8.4 Hz, 1H), 7.74 (s, 1H), |
| | | 7.62-7.38 (m, 4H), 7.29-7.17 (m, 2H), 6.38 |
| | | (d, J = 2.8 Hz, 1H), 6.29 (d, J = |
| | | 5.8 Hz, 1H), 3.75-3.62 (m, 2H), |
| | | 1.70-1.55 (m, 1H), 1.54-1.42 (m, 3H), |
| | | 1.38-1.27 (m, 2H), 1.02-0.89 (m, 3H), |
| | | 0.84-0.72 (m, 2H). |
| 504 | 367.2 | 1H NMR (500 MHz, DMSO) δ 10.20 (s, 1H), |
| | | 9.74 (s, 1H), 9.04 (s, 1H), 8.90 |
| | | (d, J = 2.6 Hz, 1H), 8.79 (s, 1H), 8.00 (d, |
| | | J = 5.7 Hz, 1H), 7.86 (d, J = 8.3 Hz, |
| | | 1H), 7.66 (s, 1H), 7.54 (dd, J = 6.8, 1.5 Hz, 2H), |
| | | 7.52-7.45 (m, 1H), 7.35 (d, J = |
| | | 8.2 Hz, 1H), 6.68 (d, J = 8.3 Hz, 1H), |
| | | 6.21 (d, J = 5.7 Hz, 1H), 3.30 (s, 2H). |
| 505 | 372.3 | 1H NMR (500 MHz, DMSO) δ 9.84 (s, 1H), |
| | | 9.10 (s, 1H), 9.00 (s, 1H), 8.92 (d, |
| | | J = 2.4 Hz, 1H), 8.07 (dd, J = 9.1, 5.7 Hz, |
| | | 1H), 7.98-7.89 (m, 1H), 7.82 (s, |
| | | 1H), 7.65-7.52 (m, 2H), 7.35 (s, 1H), 7.10 (dd, |
| | | J = 8.7, 2.5 Hz, 1H), 6.78 (d, J = |
| | | 8.7 Hz, 1H), 6.31 (d, J = 5.7 Hz, 1H), 4.22 (s, 4H). |
| 506 | 513.4 | 1HNMR(500 MHz, DMSO) δ 9.27 (s, 1H), |
| | | 8.99 (s, 1H), 7.99-7.96 (m, 2H), |
| | | 7.79 (d, J = 2.3 Hz, 1H), 7.56 (d, |
| | | J = 9.0 Hz, 2H), 6.84-6.82 (m, 2H), 6.11 (d, |
| | | J = 5.7 Hz, 1H), 4.39-4.36 (m, 2H), 4.28-4.25 |
| | | (m, 2H), 3.97 (t, J = 6.3 Hz, |
| | | 2H), 3.11-3.07 (m, 4H), 2.93-2.88 |
| | | (m, 4H), 2.64-2.61 (m, 2H), 1.89-1.84 |
| | | (m, 2H). |

### II. Biologic and Pharmacologic Assays and Methods

The activity of the compounds as IGF-1R inhibitors is illustrated in the following assays.

### Example B-1: IGF-1R Assay

50K PC3 cells were seeded on day 1 in 50 μL of media in 96-well plate and incubated overnight at 37° C., 5% CO₂. On day 2, all cells were expected to be at 80-90% confluency. Cells were starved for two hours by changing the media to 90 μL of EMEM serum-free media and incubated for 2 hours. Next, cells were treated with 10 μL of 10× concentrated and 3× serially diluted compounds for two hours. It makes 1× of compounds in 100 μL media. This was followed by stimulation of cells with 11×IGF1 for 30 min in 10 μL of serum-free EMEM media at the final concentration of 200 ng/mL.

The media was removed and replaced by 50 μL of 1× lysis buffer from the AlphaLISA kit (with 1× of cOmplete™ protease inhibitor). The plates were sealed, and cells were allowed to lyse for 10 min in the shaker before storing the plate at −20° C. until used. The AlphaLISA assay with processed cell lysates were performed as described in the protocol. Briefly, 10 μL of the undiluted sample were loaded to 384 well Optiplates and 5 μL of the acceptor mix (as described in the AlphaLISA kit) was added to each well. The plates were sealed and briefly vortexed followed by the incubation at RT for 3 hours. In the dark, 5 μL of donor mix was added to each well. All plates were sealed and stored at RT overnight. The plates were read in EnVision reader using standard AlphaLISA settings.

Compound activity was analyzed using CBIS data analysis suite (ChemInnovation, CA). Percentage inhibition was calculated using the following formula: % Inhibition=100%×(1−(mean RLU of test sample−mean RLU of vehicle control)/(mean RLU of EC80 control−mean RLU of vehicle control)). The activity for representative examples of the invention is shown in the following table, wherein A: IC₅₀≤500 nM; B: 500 nM<IC₅₀≤2 μM; C: 2 μM<IC₅₀≤10 μM; D: IC₅₀>10 μM.

TABLE B1
Biological Activity against IGF-1R

| Cmpd # | IC₅₀ |
|---|---|
| 1 | A |
| 2 | A |
| 3 | A |
| 4 | C |
| 5 | A |
| 6 | A |
| 7 | A |
| 8 | A |
| 9 | A |
| 10 | A |
| 11 | A |
| 12 | A |
| 14 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 56 | A |
| 57 | A |
| 60 | B |
| 61 | A |
| 62 | A |
| 63 | A |
| 64 | A |
| 65 | A |
| 66 | B |
| 67 | B |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | B |
| 83 | A |
| 84 | B |
| 85 | B |
| 86 | A |
| 87 | A |
| 88 | A |
| 89 | B |
| 90 | A |
| 91 | A |
| 92 | A |
| 94 | A |
| 95 | A |
| 96 | A |
| 97 | A |
| 98 | A |
| 99 | A |
| 101 | A |
| 102 | A |
| 103 | A |
| 104 | A |
| 105 | A |
| 106 | B |
| 107 | A |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | A |
| 123 | A |
| 124 | A |
| 125 | A |
| 126 | B |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | B |
| 143 | A |
| 144 | A |
| 145 | A |
| 146 | A |
| 147 | B |
| 148 | A |
| 149 | A |
| 150 | B |
| 151 | A |
| 152 | A |
| 153 | A |
| 154 | A |
| 155 | A |
| 156 | A |
| 157 | B |
| 158 | B |
| 159 | A |
| 160 | A |
| 161 | B |
| 162 | A |
| 163 | A |
| 164 | A |
| 165 | B |
| 166 | B |
| 167 | A |
| 168 | A |
| 169 | A |
| 170 | B |
| 171 | A |
| 172 | A |
| 173 | A |
| 174 | B |
| 175 | A |
| 176 | A |
| 177 | A |
| 178 | A |
| 179 | A |
| 180 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | B |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | A |
| 234 | A |
| 235 | A |
| 236 | A |
| 237 | A |
| 238 | A |
| 239 | A |
| 240 | D |
| 241 | A |
| 242 | C |
| 243 | A |
| 244 | A |
| 245 | A |
| 246 | A |
| 247 | A |
| 248 | A |
| 249 | A |
| 250 | A |
| 251 | A |
| 252 | C |
| 253 | D |
| 254 | A |
| 255 | A |
| 256 | A |
| 257 | A |
| 258 | A |
| 259 | A |
| 260 | A |
| 261 | A |
| 262 | A |
| 263 | A |
| 264 | D |
| 265 | C |
| 266 | A |
| 267 | A |
| 268 | A |
| 269 | D |
| 270 | A |
| 271 | A |
| 272 | A |
| 273 | A |
| 274 | C |
| 275 | A |
| 276 | A |
| 277 | A |
| 278 | A |
| 279 | B |
| 280 | A |
| 281 | A |
| 282 | A |
| 283 | A |
| 284 | A |
| 285 | A |
| 286 | A |
| 288 | B |
| 289 | B |
| 290 | A |
| 291 | A |
| 292 | C |
| 293 | D |
| 294 | C |
| 295 | C |
| 296 | D |
| 297 | A |
| 298 | A |
| 299 | B |
| 300 | B |
| 301 | A |
| 302 | A |
| 303 | A |
| 304 | D |
| 306 | D |
| 307 | C |
| 308 | A |
| 309 | A |
| 310 | A |
| 311 | A |
| 312 | A |
| 313 | A |
| 314 | D |
| 315 | A |
| 316 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | A |
| 338 | A |
| 339 | A |
| 340 | A |
| 341 | B |
| 342 | A |
| 343 | A |
| 344 | A |
| 345 | B |
| 346 | B |
| 347 | A |
| 348 | A |
| 349 | B |
| 350 | C |
| 351 | B |
| 352 | C |
| 353 | C |
| 354 | C |
| 355 | B |
| 356 | B |
| 357 | A |
| 358 | A |
| 359 | A |
| 360 | A |
| 361 | A |
| 363 | A |
| 364 | A |
| 365 | A |
| 366 | A |
| 367 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | C |
| 381 | A |
| 383 | A |
| 385 | B |
| 386 | B |
| 387 | A |
| 388 | A |
| 389 | A |
| 390 | B |
| 391 | C |
| 392 | A |
| 393 | A |
| 394 | A |
| 395 | C |
| 396 | C |
| 397 | C |
| 398 | A |
| 399 | C |
| 400 | B |
| 401 | A |
| 402 | A |
| 403 | C |
| 404 | C |
| 405 | C |
| 406 | C |
| 407 | B |
| 408 | A |
| 409 | C |
| 410 | C |
| 411 | C |
| 412 | A |
| 413 | A |
| 414 | A |
| 415 | C |
| 416 | A |
| 417 | B |
| 418 | A |
| 419 | A |
| 421 | A |
| 422 | A |
| 423 | A |
| 424 | B |
| 425 | A |
| 426 | A |
| 427 | A |
| 428 | A |
| 429 | A |
| 430 | A |
| 431 | A |
| 432 | A |
| 433 | B |
| 443 | A |
| 444 | A |
| 447 | A |
| 448 | A |
| 449 | A |
| 450 | B |
| 451 | A |
| 452 | A |
| 453 | A |
| 454 | A |
| 456 | A |
| 457 | A |
| 459 | A |
| 461 | A |
| 462 | A |
| 463 | A |
| 464 | A |
| 465 | A |
| 466 | A |
| 467 | A |
| 468 | A |
| 469 | A |
| 470 | A |
| 470 | B |
| 471 | D |
| 472 | A |
| 473 | A |
| 474 | B |
| 475 | B |
| 476 | A |
| 477 | A |
| 478 | A |
| 479 | A |
| 480 | A |
| 481 | A |
| 482 | A |
| 483 | A |
| 484 | A |
| 485 | A |
| 486 | A |
| 487 | A |
| 488 | A |
| 489 | A |
| 490 | A |
| 491 | A |
| 492 | A |
| 493 | A |
| 494 | A |
| 495 | A |
| 496 | A |
| 497 | A |
| 498 | B |
| 499 | B |
| 500 | C |
| 501 | B |
| 502 | A |
| 503 | C |
| 504 | |
| 505 | A |
| 506 | A |

### Example B-2: IGF-1R Assay

PathHunter cell lines were expanded from freezer stocks according to standard procedures. Cells were seeded in a total volume of 20 μL into white walled, 384-well microplates and incubated for the appropriate time prior to testing.

Cells are pre-incubated with compound followed by EC80 agonist challenge as follows:
1. Intermediate dilution of sample stocks was performed to generate 5× sample in assay buffer.
2. 5 μL of 5× sample was added to cells and incubated at 37° C. for 60 minutes. Vehicle concentration was 1%.
3. 5 μL of 6×EC80 agonist in assay buffer was added to the cells and incubated at room temperature for 3 hours.

Assay signal was generated through a single addition of 12.5 or 15 μL (50% v/v) of PathHunter Detection reagent cocktail for agonist and antagonist assays respectively, followed by a one hour incubation at room temperature. Microplates were read following signal generation with a PerkinElmer Envision™ instrument for chemilumine-scent signal detection.

Compound activity was analyzed using CBIS data analysis suite (ChemInnovation, CA). Percentage inhibition was calculated using the following formula: % Inhibition=100%×(1−(mean RLU of test sample−mean RLU of vehicle control)/(mean RLU of EC80 control−mean RLU of vehicle control)). The activity for representative examples of the invention is shown in the following table, wherein A: IC₅₀≤500 nM; B: 500 nM<IC₅₀≤2 μM; C: 2 M<IC₅₀≤10 μM; D: IC₅₀>10 M.

TABLE B2
Biological Activity against IGF-1R

| Cmpd # | IC₅₀ |
|---|---|
| 3 | A |
| 10 | B |
| 17 | A |
| 21 | A |
| 77 | A |
| 92 | A |
| 94 | A |
| 109 | B |
| 115 | B |
| 116 | B |
| 129 | A |
| 130 | A |
| 134 | A |
| 135 | A |
| 136 | A |
| 137 | A |
| 138 | A |
| 139 | A |
| 140 | A |
| 141 | A |
| 142 | B |
| 143 | A |
| 144 | A |
| 145 | A |
| 146 | A |
| 147 | B |
| 148 | A |
| 149 | A |
| 150 | B |
| 152 | A |
| 153 | A |
| 159 | B |
| 162 | B |
| 169 | A |
| 170 | B |
| 171 | A |
| 177 | A |
| 178 | A |
| 195 | A |
| 196 | A |
| 200 | A |
| 204 | A |
| 211 | A |
| 212 | B |
| 213 | A |
| 214 | A |
| 215 | A |
| 216 | A |
| 217 | D |
| 218 | B |
| 219 | D |
| 220 | B |
| 221 | B |
| 222 | A |
| 223 | B |
| 224 | B |
| 291 | B |
| 297 | B |
| 357 | A |
| 466 | B |
| 496 | B |
| 497 | A |
| 498 | B |
| 499 | B |
| 500 | B |
| 501 | B |
| 502 | A |
| 503 | C |
| 505 | A |

### III. Pharmaceutical Compositions

### Example C-1: Oral Solution

To prepare a pharmaceutical composition for oral delivery, a sufficient amount of a compound disclosed herein, or a pharmaceutically acceptable salt or solvate thereof, is added to water (with optional solubilizer(s), optional buffer(s) and taste masking excipients) to provide a 20 mg/mL solution.

### Example C-2: Oral Tablet

A tablet is prepared by mixing 20-50% by weight of a compound disclosed herein, or a pharmaceutically acceptable salt or solvate thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example C-3: Oral Capsule

To prepare a pharmaceutical composition for oral delivery, 10-500 mg of a compound disclosed herein, or a pharmaceutically acceptable salt or solvate thereof, is mixed with starch or other suitable powder blend. The mixture is incorporated into an oral dosage unit such as a hard gelatin capsule, which is suitable for oral administration.

In another embodiment, 10-500 mg of a compound disclosed herein, or a pharmaceutically acceptable salt or solvate thereof, is placed into size 4 capsule, or size 1 capsule (hypromellose or hard gelatin) and the capsule is closed.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A quinolone compound that has the structure of Formula (I), or a pharmaceutically acceptable salt thereof: 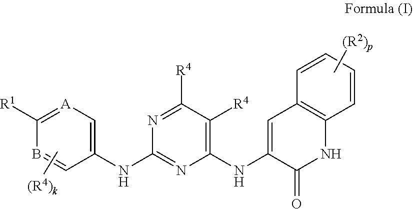
wherein:
R¹ is unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —OR²⁰, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
or R¹ is 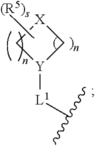
each n is independently 1, 2, or 3;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
Y is CH; and L¹ is —O—, —S—, —N(R²¹)—, -(unsubstituted or substituted C₁-C₆alkyl)-O—, -(unsubstituted or substituted C₁-C₆alkyl)-S—, or -(unsubstituted or substituted C₁-C₆alkyl)-N(R²¹)—;
or Y is N; and L¹ is -(unsubstituted or substituted C₁-C₆alkyl)-O—, -(unsubstituted or substituted C₁-C₆alkyl)-S—, or -(unsubstituted or substituted C₁-C₆alkyl)-N(R²¹)—;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
or both R⁸ are taken together with the carbon to which they are attached to form a unsubstituted or substituted C₃-C₈cycloalkyl, unsubstituted or substituted C₃-C₈heterocycloalkyl;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
B is C(R³) or N;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R², —CO₂R²¹, —N(R²¹)₂, —SR²⁰, —S(═O)R²⁰, or —SO₂R²⁰;
or R¹ and R³ are taken together with the intervening carbon atoms connecting R¹ to R³ to form a ring A that is monocyclic C₃-C₈cycloalkyl, monocyclic C₃-C₈heterocycloalkyl, bicyclic C₅-C₁₀cycloalkyl, or bicyclic C₅-C₁₀heterocycloalkyl, wherein ring A is unsubstituted or substituted with 1 or 2 R¹⁴, or 1 or 2 R¹⁵;
or R¹ and R³ are taken together with the intervening carbon atoms connecting R¹ to R³ to form a ring A that is monocyclic C₃-C₈cycloalkyl, bicyclic C₅-C₁₀cycloalkyl, monocyclic C₃-C₈heterocycloalkyl, or bicyclic C₅-C₁₀heterocycloalkyl, wherein ring A is unsubstituted or substituted with one or more groups independently selected from the group consisting of R¹⁴, R¹⁵, R¹⁶, and R¹⁷;
each R¹⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, substituted or unsubstituted C₁-C₆alkyl-N(R²¹)₂, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R¹⁵ is -substituted or unsubstituted C₁-C₆alkyl, -substituted or unsubstituted C₁-C₆alkyl-N(R²¹)₂, —C(═O)-substituted or unsubstituted C₁-C₆alkyl-N(R²¹)₂, -substituted or unsubstituted C₁-C₆alkyl-carbocycle, or heterocycle;
each X^{a} is independently —C(R¹⁶)₂—, —N(R¹⁷)—, —O—, or —S—;
each R¹⁶ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹⁷ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —C(═O)R²¹, —CO₂R²¹, or —C(═O)N(R²¹)₂;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
p is 0, 1, 2, 3, or 4;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

2. The compound of claim 1, or a pharmaceutically acceptable salt thereof, wherein:
R¹ is unsubstituted or substituted C₃-C₈cycloalkyl, unsubstituted or substituted C₃-C₈heterocycloalkyl, or —OR²⁰;
or R¹ is 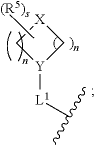
R³ is hydrogen, unsubstituted or substituted C₁-C₆alkyl, unsubstituted or substituted C₁-C₆fluoroalkyl, unsubstituted or substituted C₁-C₆heteroalkyl, or —O—C₁-C₆alkyl;
or R¹ and R³ are taken together with the intervening carbon atoms connecting R¹ to R³ to form a ring A that is monocyclic C₃-C₈heterocycloalkyl or bicyclic C₅-C₁₀heterocycloalkyl, wherein ring A is substituted with 1 or 2 R¹⁴, or 1 or 2 R¹⁵;
or R¹ and R³ are taken together with the intervening carbon atoms connecting R¹ to R³ to form a ring A that is 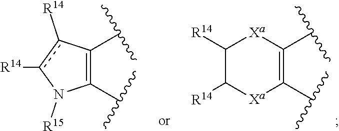
wherein [symbol] indicates a single or double bond;
each n is independently 1 or 2;
each R⁵ is independently hydrogen, halogen, or unsubstituted or substituted C₁-C₆alkyl;
s is 0, 1, or 2;
Y is CH; and L¹ is —O— or —N(R²¹)—;
X is —C(R⁸)₂—, —N(R⁹)—, or —O—;
each R⁸ is independently hydrogen, unsubstituted or substituted C₁-C₆alkyl, unsubstituted or substituted C₁-C₆fluoroalkyl, —C(═O)N(R²¹)₂, —N(R²¹)₂, or —NR²¹C(═O)R²¹;
R⁹ is hydrogen or C₁-C₆alkyl;
each R¹⁴ is independently hydrogen, halogen, unsubstituted or substituted C₁-C₆alkyl, or -(unsubstituted or substituted C₁-C₆alkyl)-N(R²¹)₂;
each R¹⁵ is unsubstituted or substituted C₁-C₆alkyl, -(unsubstituted or substituted C₁-C₆alkyl)-N(R²¹)₂, or —C(═O)-(unsubstituted or substituted C₁-C₆alkyl)-N(R²¹)₂,
each X^{a} is independently —C(R¹⁶)₂—, —N(R¹⁷)—, or —O—;
each R¹⁶ is independently hydrogen or unsubstituted or substituted C₁-C₆alkyl;
each R¹⁷ is independently hydrogen or unsubstituted or substituted C₁-C₆alkyl; and
each R²¹ is independently selected from hydrogen or unsubstituted or substituted C₁-C₆alkyl.

3. The compound of claim 1, or a pharmaceutically acceptable salt thereof, wherein:
R¹ is 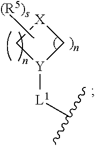
R³ is hydrogen, unsubstituted or substituted C₁-C₆alkyl, unsubstituted or substituted C₁-C₆fluoroalkyl, unsubstituted or substituted C₁-C₆heteroalkyl, or —O—C₁-C₆alkyl;
each n is independently 1 or 2;
each R⁵ is independently hydrogen, halogen, or unsubstituted or substituted C₁-C₆alkyl;
s is 0, 1, or 2;
Y is CH; and L¹ is —O— or —N(R²¹)—;
X is —C(R⁸)₂—, —N(R⁹)—, or —O—;
each R⁸ is independently hydrogen, unsubstituted or substituted C₁-C₆alkyl, unsubstituted or substituted C₁-C₆fluoroalkyl, —C(═O)N(R²¹)₂, —N(R²¹)₂, or —NR²¹C(═O)R²¹;
R⁹ is hydrogen or C₁-C₆alkyl; and
each R²¹ is independently selected from hydrogen or unsubstituted or substituted C₁-C₆alkyl.

4. The compound of claim 1, or a pharmaceutically acceptable salt thereof, wherein:
R¹ is

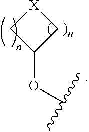

5. The compound of claim 1, or a pharmaceutically acceptable salt thereof, wherein:
R¹ is

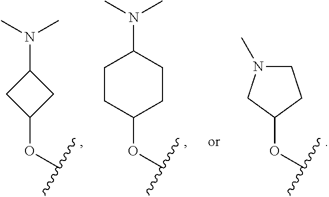

6. The compound of claim 1, or a pharmaceutically acceptable salt thereof, wherein:
B is C(R³); and
R¹ is substituted C₃-C₈cycloalkyl, unsubstituted or substituted C₃-C₈heterocycloalkyl, or O-(substituted C₁-C₆alkyl).

7. The compound of claim 6, or a pharmaceutically acceptable salt thereof, wherein:
R¹ is substituted C₃-C₈heterocycloalkyl, wherein C₃-C₈heterocycloalkyl contains 0-4 N atoms, and 0-2 O atoms.

8. The compound of claim 6, or a pharmaceutically acceptable salt thereof, wherein:
R¹ is

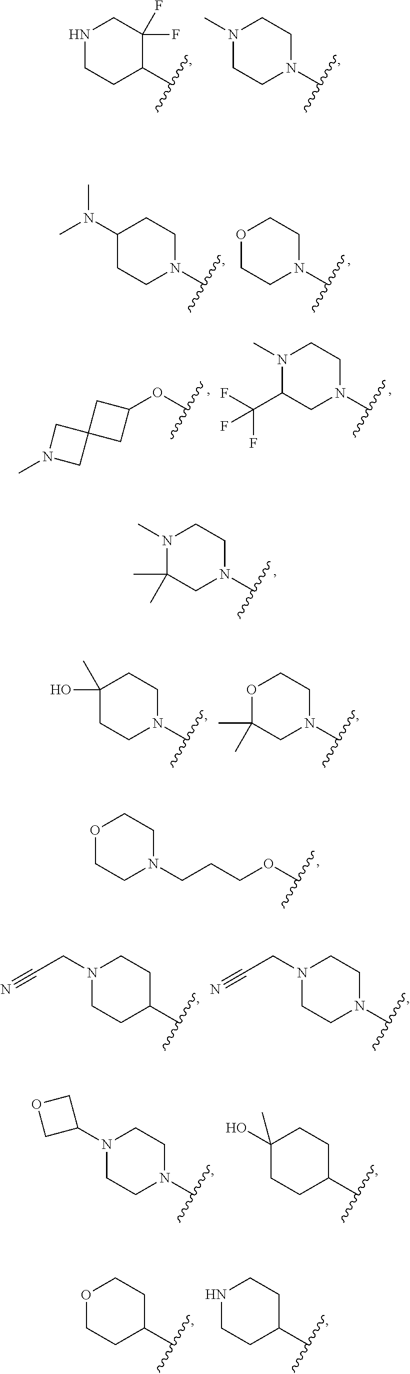

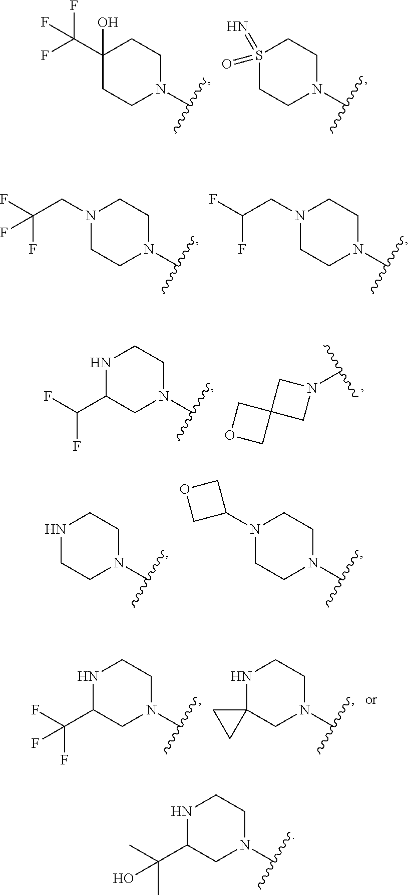

9. The compound of claim 1, or a pharmaceutically acceptable salt thereof, wherein:
B is C(R³); and
R³ is hydrogen or unsubstituted or substituted C₁-C₆alkoxy.

10. The compound of claim 1, or a pharmaceutically acceptable salt thereof, wherein:
B is C(R³);
R¹ and R³ are taken together with the intervening carbon atoms connecting R¹ to R³ to form a ring A that is 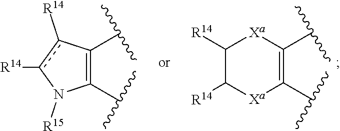
wherein [symbol] indicates a single or double bond;
each R⁵ is independently hydrogen, halogen, or C₁-C₆alkyl;
s is 0, 1, or 2;
Y is CH; and L¹ is —O— or —N(R²¹)—;
X is —C(R⁸)₂—, —N(R⁹)—, or —O—,
each R⁸ is independently hydrogen, unsubstituted or substituted C₁-C₆alkyl, unsubstituted or substituted C₁-C₆fluoroalkyl, —C(═O)N(R²¹)₂, —N(R²¹)₂, or —NR²¹C(═O)R²¹;
R⁹ is hydrogen or C₁-C₆alkyl;
each R¹⁴ is independently hydrogen, halogen, C₁-C₆alkyl, or —C₁-C₆alkyl-N(R²¹)₂;
each R¹⁵ is —C₁-C₆alkyl, —C₁-C₆alkyl-N(R²¹)₂, or —C(═O)—C₁-C₆alkyl-N(R²¹)₂,
each X^{a} is independently —C(R¹⁶)₂—, —N(R¹⁷)—, or —O—;
each R¹⁶ is independently hydrogen or unsubstituted or substituted C₁-C₆alkyl;
each R¹⁷ is independently hydrogen or unsubstituted or substituted C₁-C₆alkyl; and
each R²¹ is independently selected from hydrogen or unsubstituted or substituted C₁-C₆alkyl.

11. The compound of claim 1, or a pharmaceutically acceptable salt thereof, wherein:
B is C(R³);
R¹ and R³ are taken together with the intervening carbon atoms connecting R¹ to R³ to form a ring A that is monocyclic C₃-C₈heterocycloalkyl or bicyclic C₅-C₁₀heterocycloalkyl, wherein ring A is substituted with 1 or 2 R¹⁴, or 1 or 2 R¹⁵;
each R¹⁴ is independently hydrogen, halogen, C₁-C₆alkyl, or —C₁-C₆alkyl-N(R²¹)₂; and
each R¹⁵ is —C₁-C₆alkyl, —C₁-C₆alkyl-N(R²¹)₂, or —C(═O)—C₁-C₆alkyl-N(R²¹)₂.

12. The compound of claim 11, or a pharmaceutically acceptable salt thereof, wherein:
B is C(R³);
R¹ and R³ are taken together with the intervening carbon atoms connecting R¹ to R³ to form a ring A that is a bicyclic C₅-C₁₀heterocycloalkyl that is a fused bicyclic C₅-C₁₀heterocycloalkyl, bridged bicyclic C₅-C₁₀heterocycloalkyl, or spiro bicyclic C₅-C₁₀heterocycloalkyl.

13. The compound of claim 11, or a pharmaceutically acceptable salt thereof, wherein:
B is C(R³);
R¹ and R³ are taken together with the intervening carbon atoms connecting R¹ to R³ to form a ring A that is a bicyclic C₅-C₁₀heterocycloalkyl that is a spiro bicyclic C₅-C₁₀heterocycloalkyl containing at least one N atom in the ring.

14. The compound of claim 1, or a pharmaceutically acceptable salt thereof, wherein:
B is C(R³); and
R¹ and R³ are taken together with the intervening carbon atoms connecting R¹ to R³ to form a ring A, wherein 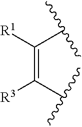
is

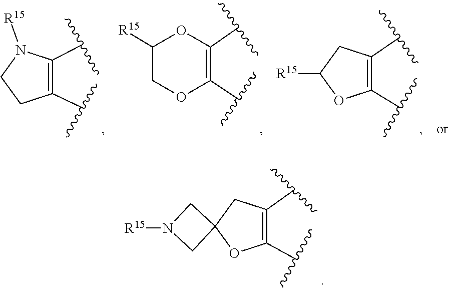

15. The compound of claim 14, or a pharmaceutically acceptable salt thereof, wherein:
B is C(R³); and
R¹ and R³ are taken together with the intervening carbon atoms connecting R¹ to R³ to form a ring A, wherein 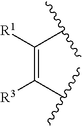
is

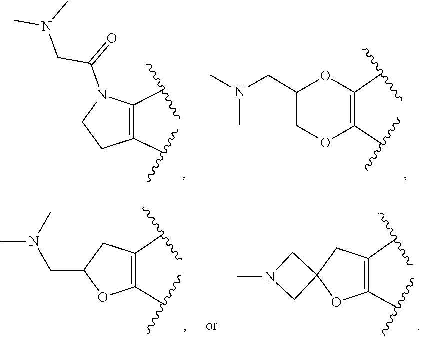 [SMILES: *C1=C(*)N(C(=O)CN(C)C)CC1 ; *C1=C(*)OC(CN(C)C)CO1 ; *C1=C(*)OC(CN(C)C)C1 ; *C1=C(*)OC2(C1)CN(C)C2]

16. The compound of claim 1, or a pharmaceutically acceptable salt thereof, wherein:
B is C(R³); and
R¹ is substituted C₃-C₈cycloalkyl, unsubstituted or substituted C₃-C₈heterocycloalkyl, or O-(substituted C₁-C₆alkyl).

17. The compound of claim 1, or a pharmaceutically acceptable salt thereof, wherein:
each R² is independently hydrogen, halogen, unsubstituted or substituted C₁-C₆alkyl, unsubstituted or substituted C₁-C₆fluoroalkyl, or unsubstituted or substituted C₁-C₆heteroalkyl;
p is 0, 1, or 2; and
each R⁴ is independently hydrogen, unsubstituted or substituted C₁-C₆alkyl, or unsubstituted or substituted C₁-C₆alkoxy.

18. The compound of claim 1, or a pharmaceutically acceptable salt thereof, wherein:
each R² is independently hydrogen, halogen, —CH₃, —CF₃, —CHF₂, or —CH₂F;
p is 0, 1, or 2; and
each R⁴ is independently hydrogen, —CH₃, or —OCH₃.

19. The compound of claim 1, or a pharmaceutically acceptable salt thereof, wherein:
each R² is hydrogen or halogen;
p is 0, 1, or 2; and
each R⁴ is independently hydrogen or —OCH₃.

20. The compound of claim 1, or a pharmaceutically acceptable salt thereof, wherein:

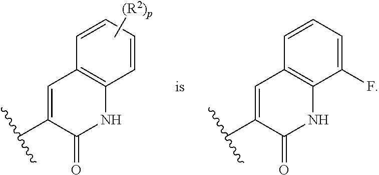

21. The compound of claim 1, wherein the compound has one of the following structures, or a pharmaceutically acceptable salt thereof:

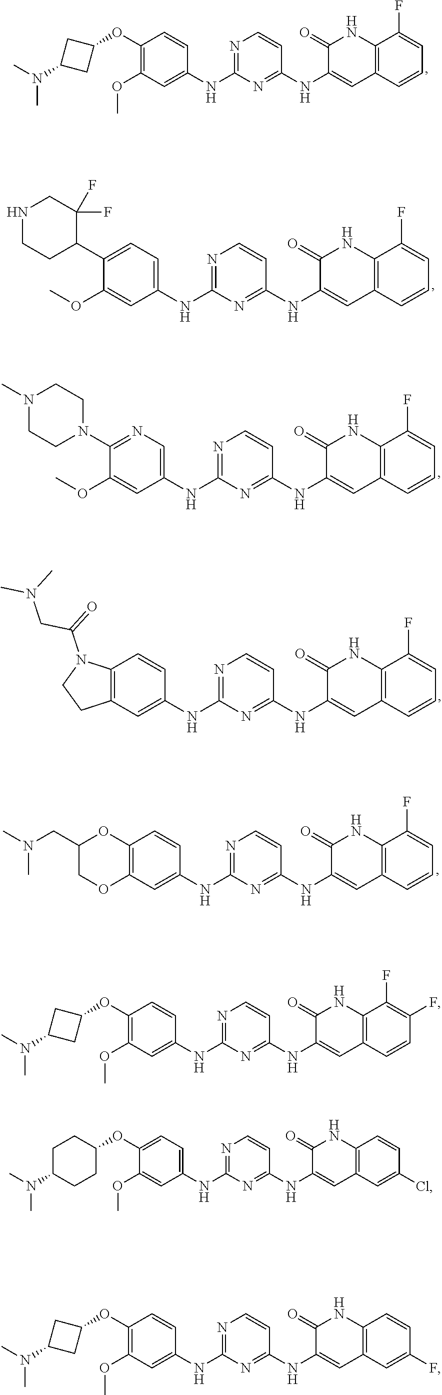

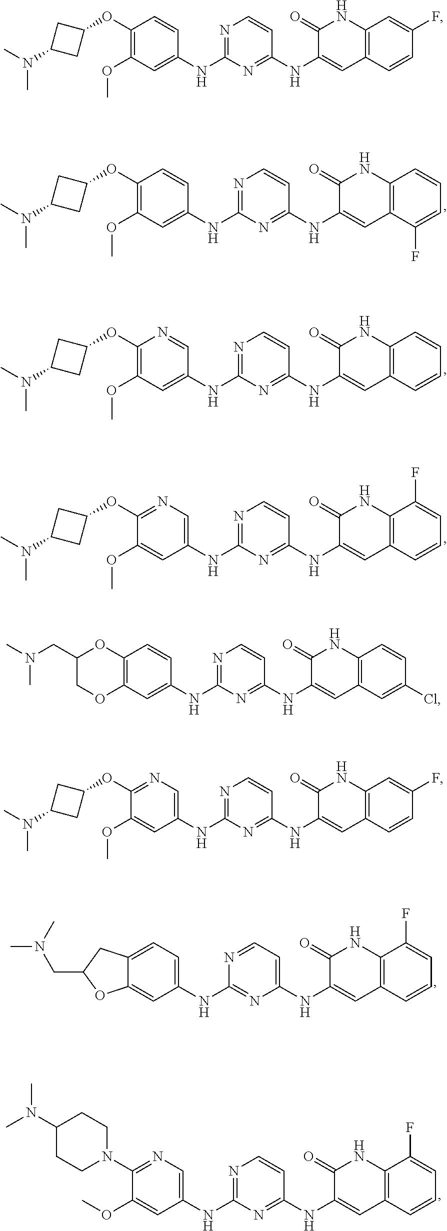

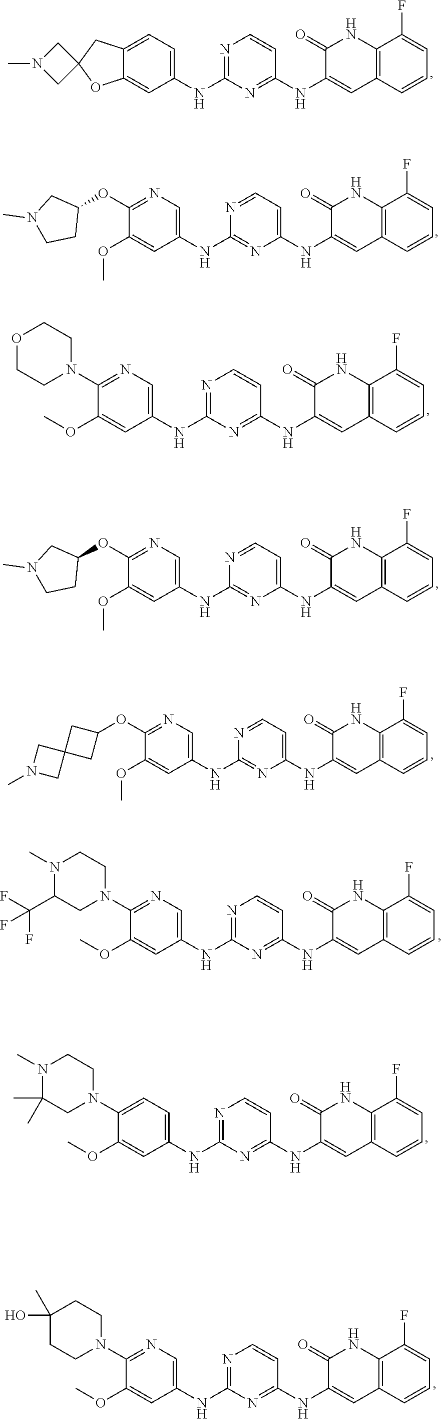

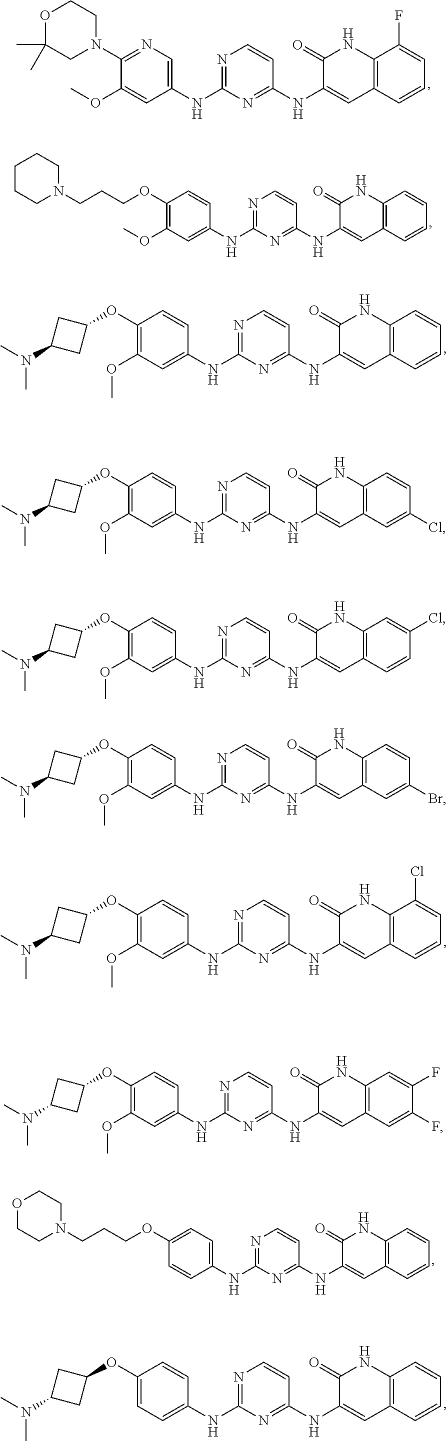

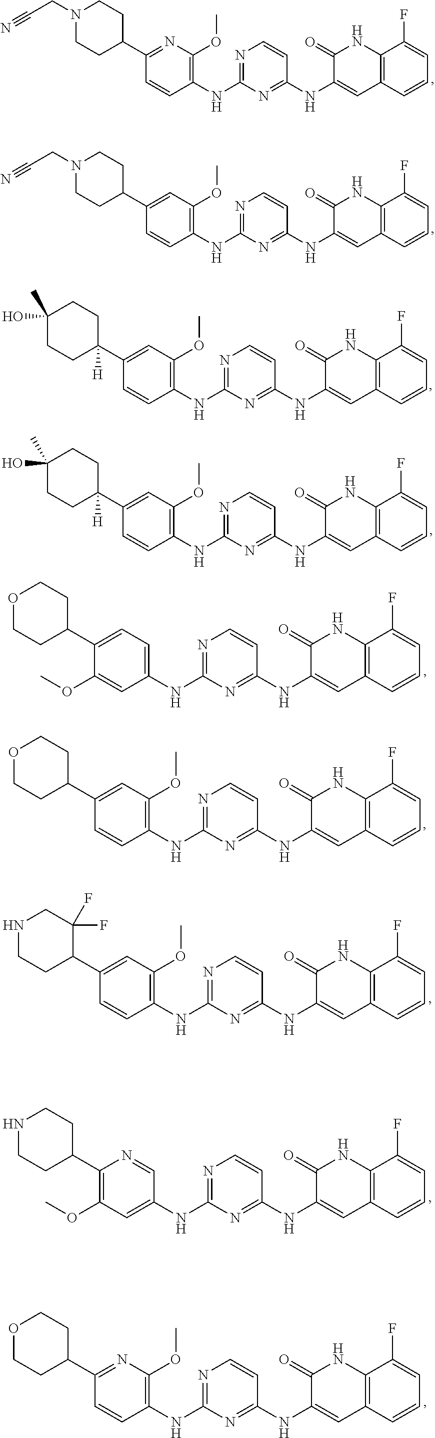

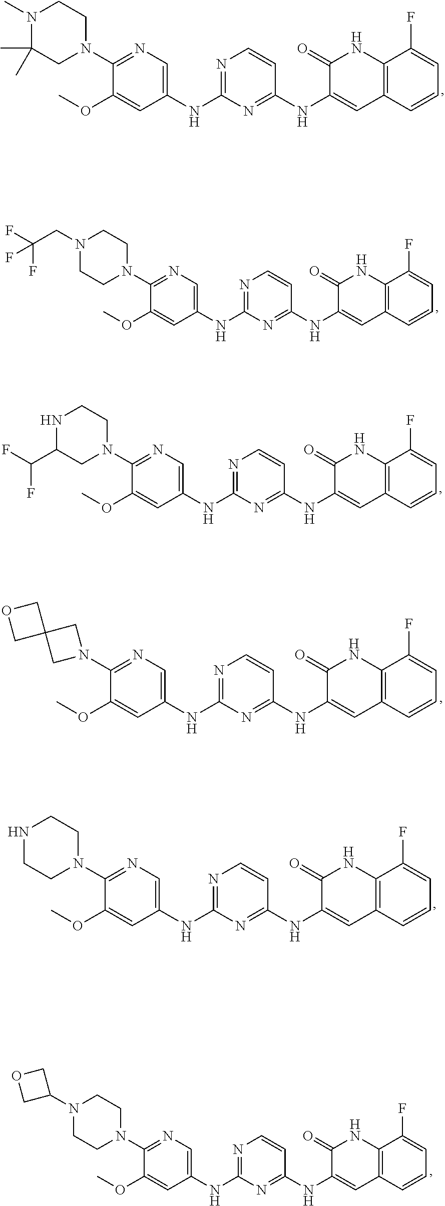 [SMILES: COc1cc(Nc2nccc(Nc3cc4cccc(F)c4[nH]c3=O)n2)cnc1N1CCN(C)C(C)(C)C1 ; COc1cc(Nc2nccc(Nc3cc4cccc(F)c4[nH]c3=O)n2)cnc1N1CCN(CC(F)(F)F)CC1 ; COc1cc(Nc2nccc(Nc3cc4cccc(F)c4[nH]c3=O)n2)cnc1N1CCNC(C(F)F)C1 ; COc1cc(Nc2nccc(Nc3cc4cccc(F)c4[nH]c3=O)n2)cnc1N1CC2(COC2)C1 ; COc1cc(Nc2nccc(Nc3cc4cccc(F)c4[nH]c3=O)n2)cnc1N1CCNCC1 ; COc1cc(Nc2nccc(Nc3cc4cccc(F)c4[nH]c3=O)n2)cnc1N1CCN(C2COC2)CC1]

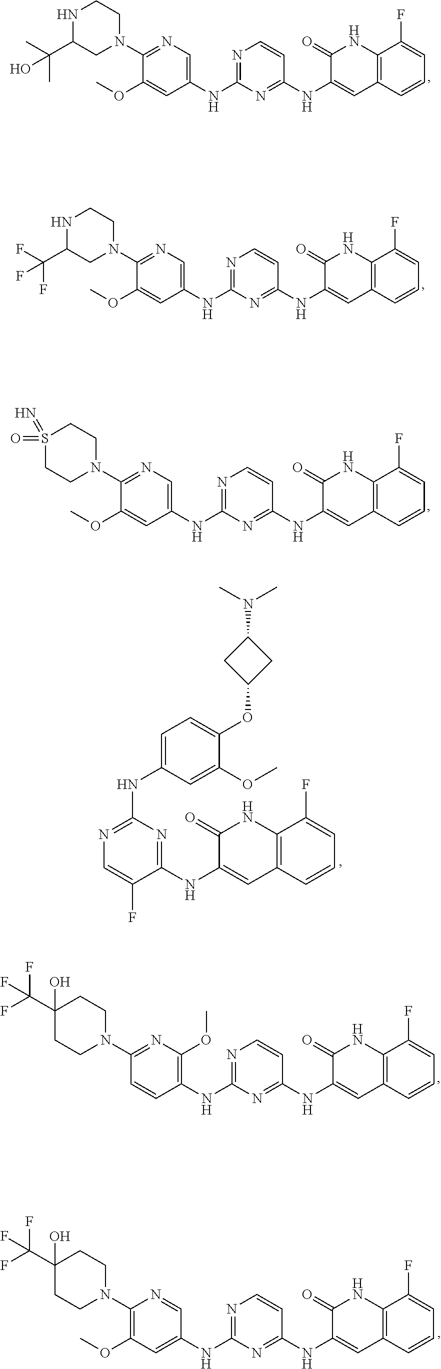 [SMILES: COc1cc(Nc2nccc(Nc3cc4cccc(F)c4[nH]c3=O)n2)cnc1N1CCNC(C(C)(C)O)C1 ; COc1cc(Nc2nccc(Nc3cc4cccc(F)c4[nH]c3=O)n2)cnc1N1CCNC(C(F)(F)F)C1 ; COc1cc(Nc2nccc(Nc3cc4cccc(F)c4[nH]c3=O)n2)cnc1N1CCS(=N)(=O)CC1 ; COc1cc(Nc2ncc(F)c(Nc3cc4cccc(F)c4[nH]c3=O)n2)ccc1O[C@H]1C[C@@H](N(C)C)C1 ; COc1nc(N2CCC(O)(C(F)(F)F)CC2)ccc1Nc1nccc(Nc2cc3cccc(F)c3[nH]c2=O)n1 ; COc1cc(Nc2nccc(Nc3cc4cccc(F)c4[nH]c3=O)n2)cnc1N1CCC(O)(C(F)(F)F)CC1]

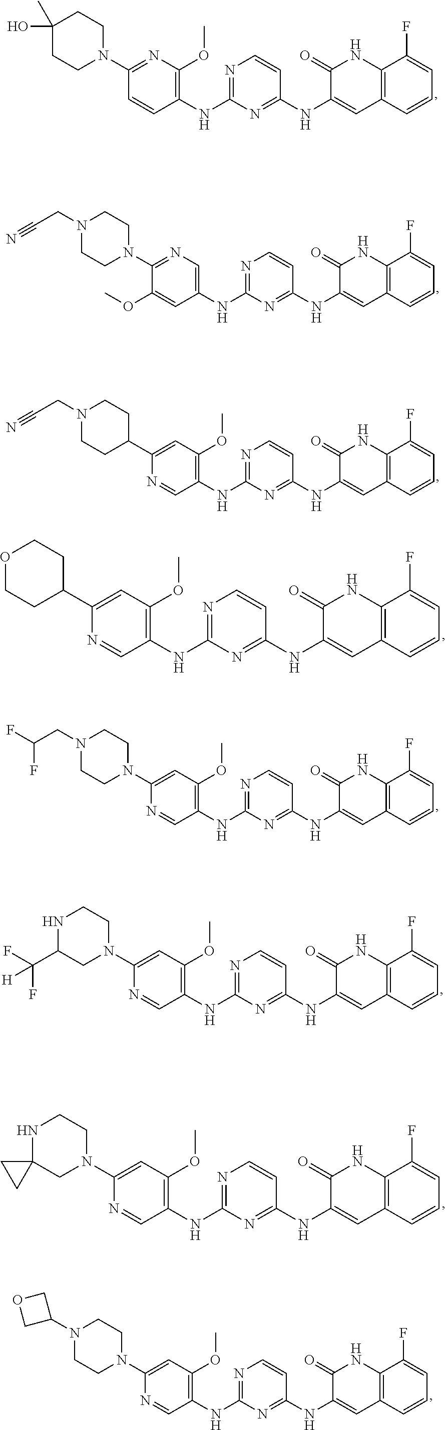

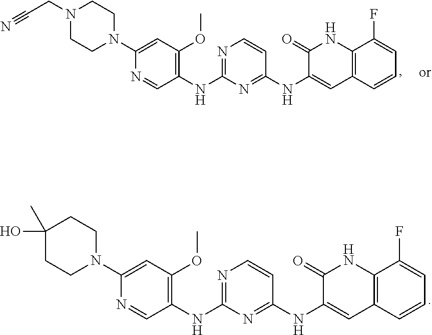 [SMILES: COc1cc(N2CCN(CC#N)CC2)ncc1Nc1nccc(Nc2cc3cccc(F)c3[nH]c2=O)n1 ; COc1cc(N2CCC(C)(O)CC2)ncc1Nc1nccc(Nc2cc3cccc(F)c3[nH]c2=O)n1]

22. A compound that has the structure of Formula (II), or a pharmaceutically acceptable salt thereof: 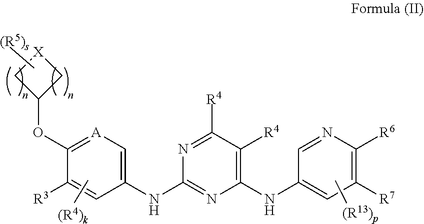
wherein:
each n is independently 1, 2, or 3;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
or both R⁸ are taken together with the carbon to which they are attached to form a unsubstituted or substituted C₃-C₈cycloalkyl, unsubstituted or substituted C₃-C₈heterocycloalkyl;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R²¹, —CO₂R²¹, —N(R²¹)₂, —SR²¹, —S(═O)R²⁰, or —SO₂R²¹;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
or R⁶ and R⁷ are taken together with the intervening carbon atoms connecting R⁶ to R⁷ to form 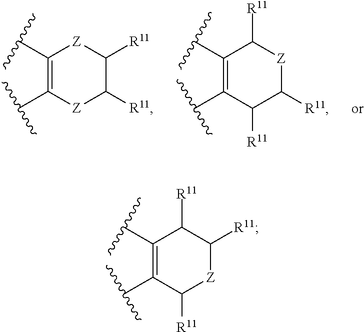
each Z is independently —C(R¹¹)₂, —N(R¹²)—, —O—, —S—, or —SO₂—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
each R¹³ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
p is 0, 1, 2, 3, or 4
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted heterocycle containing 1 or 2 N atoms, 0 or 1 O atoms, and 0 or 1 S, S(O), or S(O)₂.

23. The compound of claim 22, or a pharmaceutically acceptable salt thereof, wherein:
n is 1 or 2;
X is —C(R⁸)₂— or —N(R⁹)—;
each R⁸ is independently hydrogen, unsubstituted or substituted C₁-C₆alkyl, or —N(R²¹)₂;
R⁹ is hydrogen or unsubstituted or substituted C₁-C₆alkyl; and
s is 0.

24. The compound of claim 22, or a pharmaceutically acceptable salt thereof, wherein:
n is 1 or 2;
X is —C(R⁸)₂—;
each R⁸ is independently hydrogen or —N(R²¹)₂;
s is 0; and
R²¹ is hydrogen or C₁-C₆ alkyl, or two R²¹ on the same N atom are taken together with the N atom to which they are attached to form an unsubstituted or substituted heterocycle containing 1 or 2 N atoms, 0 or 1 O atoms, and 0 or 1 S, S(O), or S(O)₂.

25. The compound of claim 22, or a pharmaceutically acceptable salt thereof, wherein:
X is —CHN(R²¹)₂—; and
each R²¹ is independently C₁-C₆ alkyl, or two R²¹ on the same N atom are taken together with the N atom to which they are attached to form an unsubstituted or substituted monocyclic or bicyclic C₂-C₈heterocycloalkyl containing 1 or 2 N atoms, 0 or 1 O atoms, and 0 or 1 S, S(O), or S(O)₂ in the ring.

26. The compound of claim 22, or a pharmaceutically acceptable salt thereof, wherein:
X is —CHN(R²¹)₂—;
each R²¹ is independently C₁-C₄ alkyl, or two R²¹ on the same N atom are taken together with the N atom to which they are attached to form an unsubstituted or substituted monocyclic or bridged bicyclic C₂-C₈heterocycloalkyl containing 1 or 2 N atoms, 0 or 1 O atoms, and 0 or 1 S, S(O), or S(O)₂ in the ring.

27. The compound of claim 22, or a pharmaceutically acceptable salt thereof, wherein: 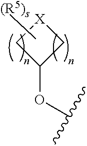
is

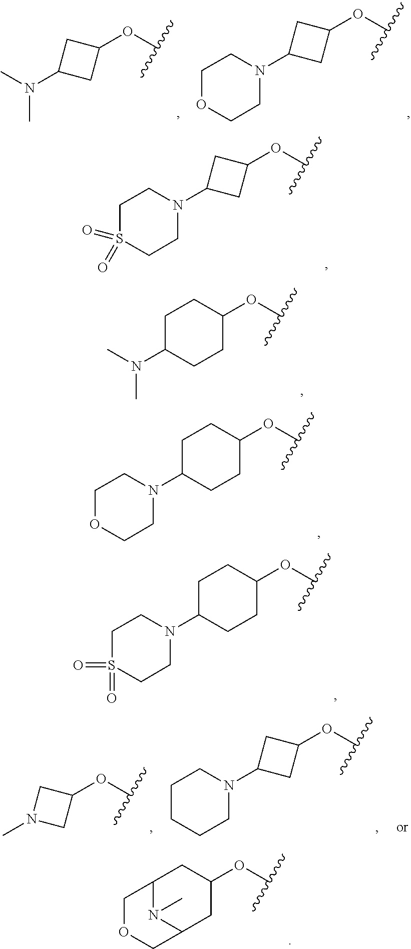

28. The compound of claim 22, or a pharmaceutically acceptable salt thereof, wherein:
each R⁴ is independently hydrogen, halogen, unsubstituted or substituted C₁-C₆alkyl, or unsubstituted or substituted C₁-C₆alkoxy; and
R³ is —O(C₁-C₆ alkyl).

29. The compound of claim 22, or a pharmaceutically acceptable salt thereof, wherein:
R⁴ is hydrogen, and
R³ is —OCH₃.

30. The compound of claim 22, or a pharmaceutically acceptable salt thereof, wherein:
R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
or R⁶ and R⁷ are taken together with the intervening carbon atoms connecting R⁶ to R⁷ to form 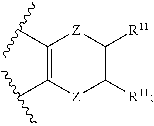
each Z is independently —C(R¹¹)₂, —N(R¹²)—, or —O—;
each R¹¹ is independently hydrogen, halogen, C₁-C₆alkyl, C₁-C₆fluoroalkyl, —CN, —OH, —OR²⁰, —N(R²¹)₂, or —NR²¹C(═O)R²¹;
each R¹³ is independently hydrogen, halogen, C₁-C₆alkyl, —CN, —OH, —OR²⁰, —CO₂R²¹, —C(═O)N(R²¹)₂, —N(R²¹)₂, or —NR²¹C(═O)R²¹; and
p is 0, 1, or 2.

31. The compound of claim 22, or a pharmaceutically acceptable salt thereof, wherein:
R⁶ and R⁷ are taken together with the intervening carbon atoms connecting R⁶ to R⁷ to form a ring B that is bicyclic C₅-C₁₀heterocycloalkyl that is a fused bicyclic C₅-C₁₀heterocycloalkyl, bridged bicyclic C₅-C₁₀heterocycloalkyl, or spiro bicyclic C₅-C₁₀heterocycloalkyl; or
R⁶ and R⁷ are taken together with the intervening carbon atoms connecting R⁶ to R⁷ to form a ring B that is bicyclic C₅-C₁₀cycloalkyl that is a fused bicyclic C₅-C₁₀cycloalkyl, bridged bicyclic C₅-C₁₀cycloalkyl, or spiro bicyclic C₅-C₁₀cycloalkyl.

32. The compound of claim 22, or a pharmaceutically acceptable salt thereof, wherein:
R⁶ and R⁷ are taken together with the intervening carbon atoms connecting R⁶ to R⁷ to form a ring B that is monocyclic C₃-C₈cycloalkyl, monocyclic C₃-C₈ aryl, or monocyclic C₃-C₈ heteroaryl, wherein ring B is unsubstituted or substituted with 1 or 2 halogen, C₁-C₆alkyl, C₁-C₆fluoroalkyl, —CN, —OH, —OR²⁰, —N(R²¹)₂, or —NR²¹C(═O)R²¹;
or R⁶ and R⁷ are taken together with the intervening carbon atoms connecting R⁶ to R⁷ to form 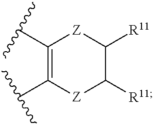
each Z is —O—;
each R¹³ is independently hydrogen, halogen, unsubstituted or substituted C₁-C₆alkyl, —CN, —OH, —OR²⁰, —CO₂R²¹, —C(═O)N(R²¹)₂; and
p is 0, 1, or 2.

33. The compound of claim 22, or a pharmaceutically acceptable salt thereof, wherein:

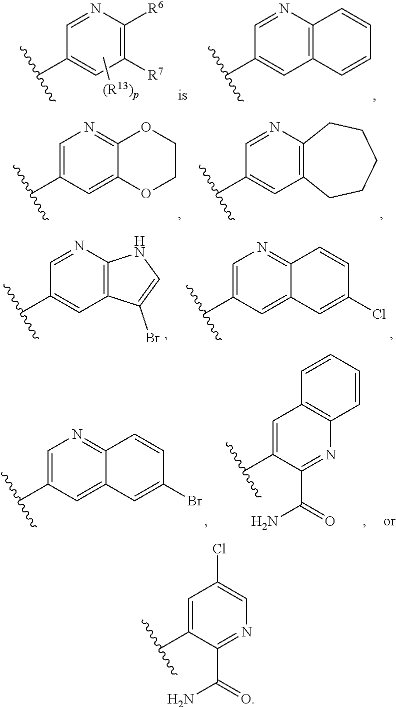

34. The compound of claim 22, wherein the compound has one of the following structures, or a pharmaceutically acceptable salt thereof:

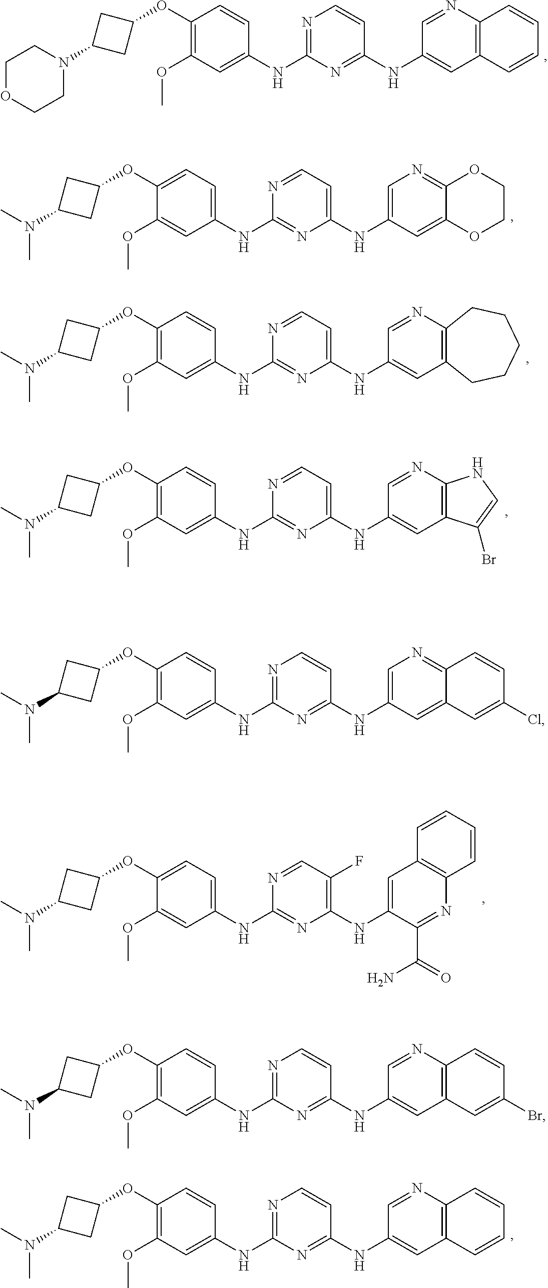

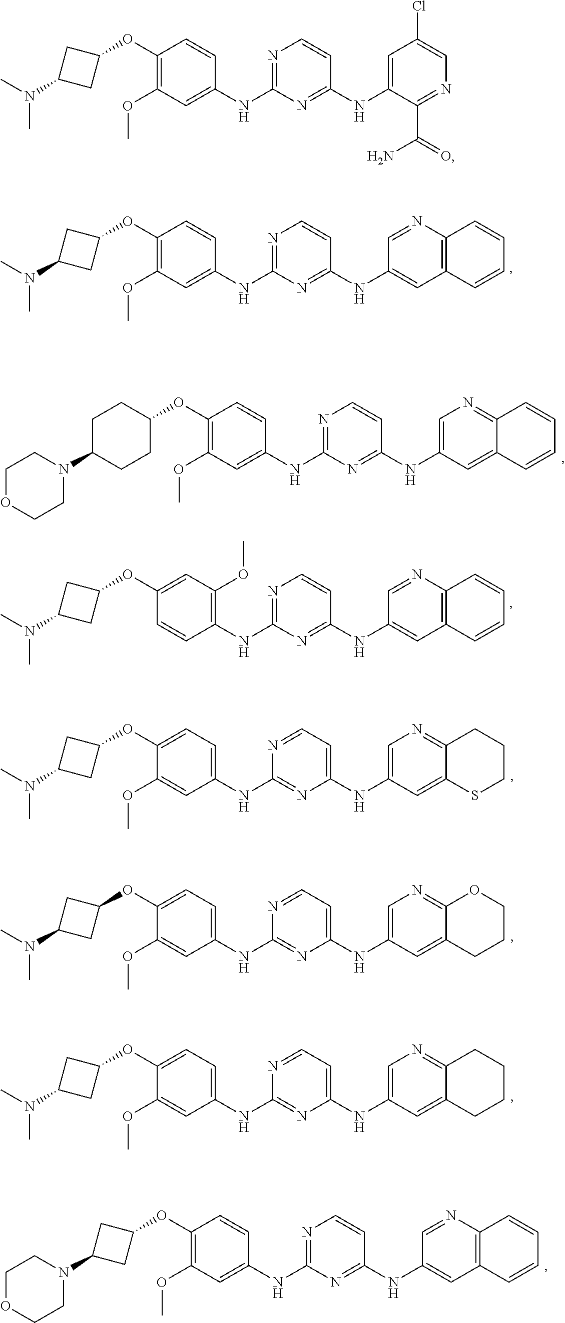

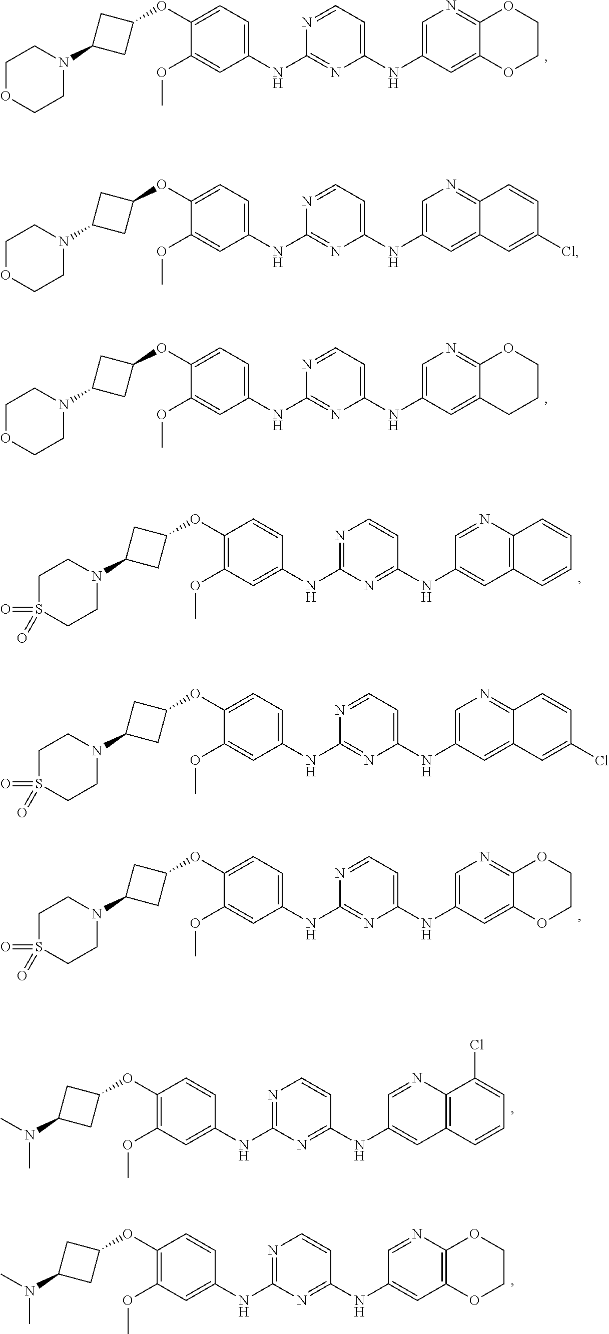

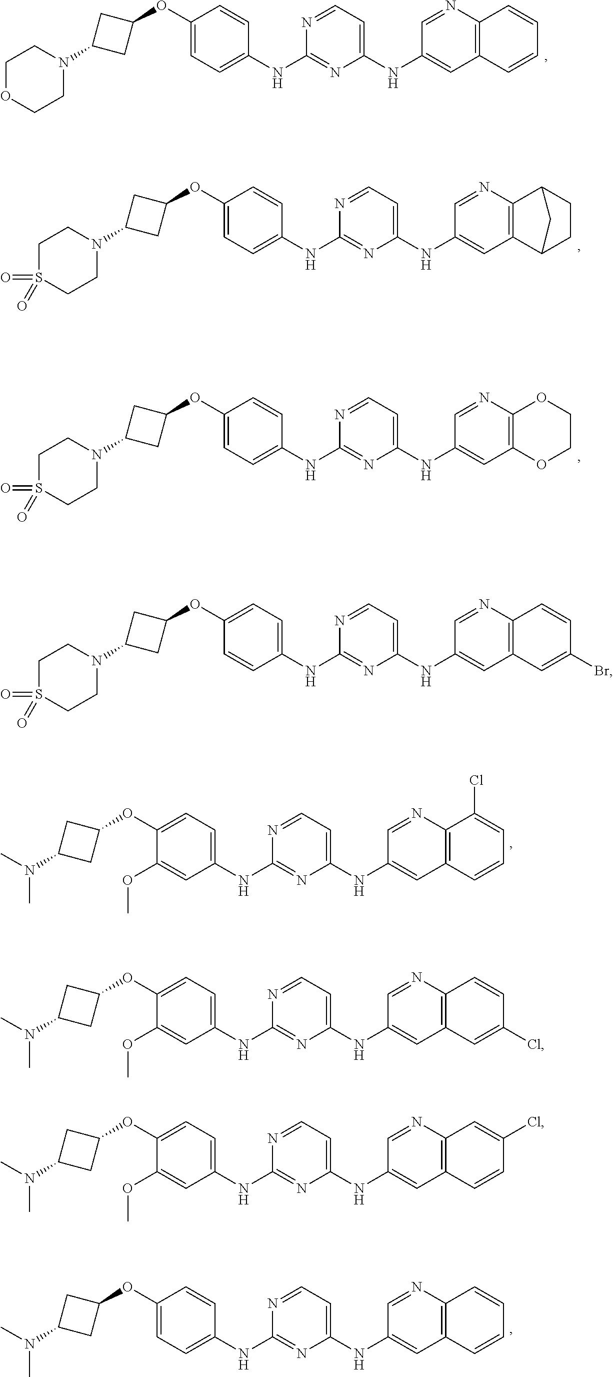

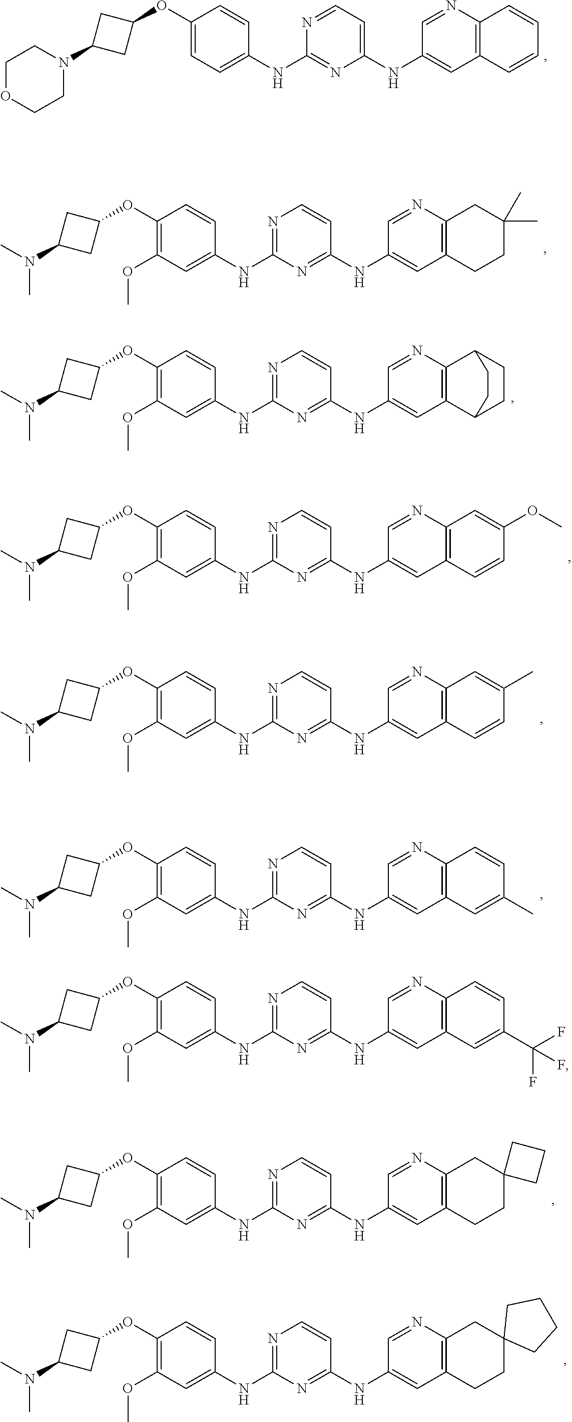

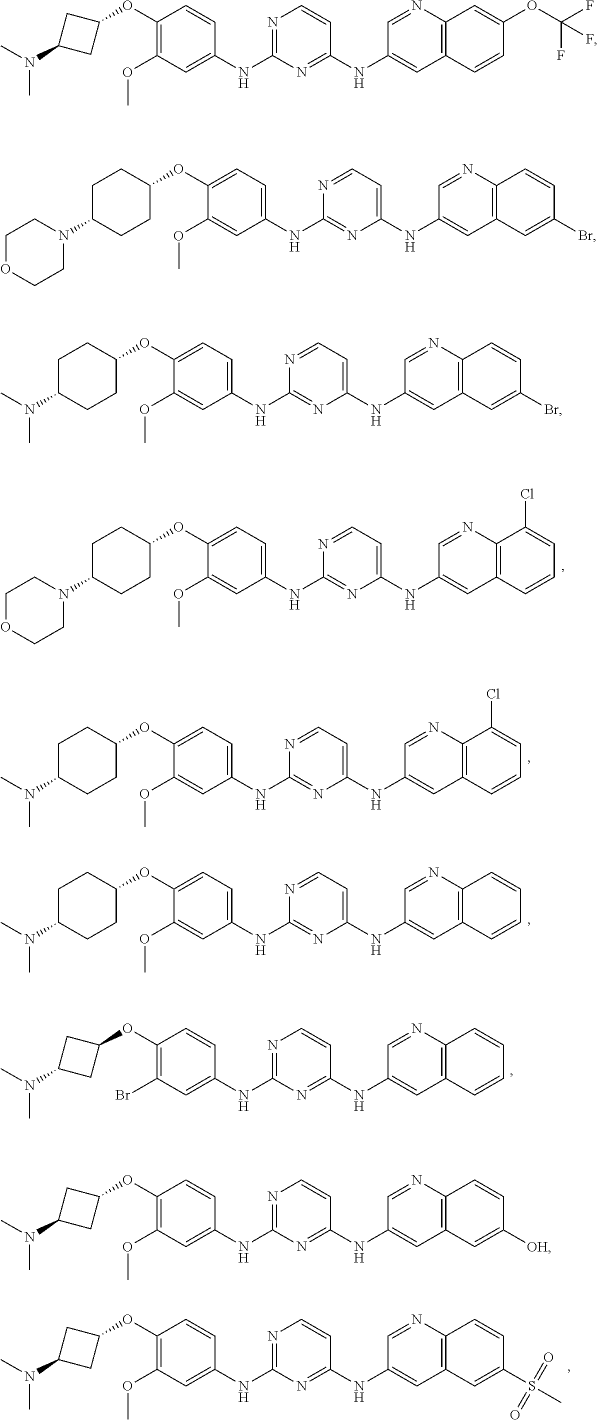

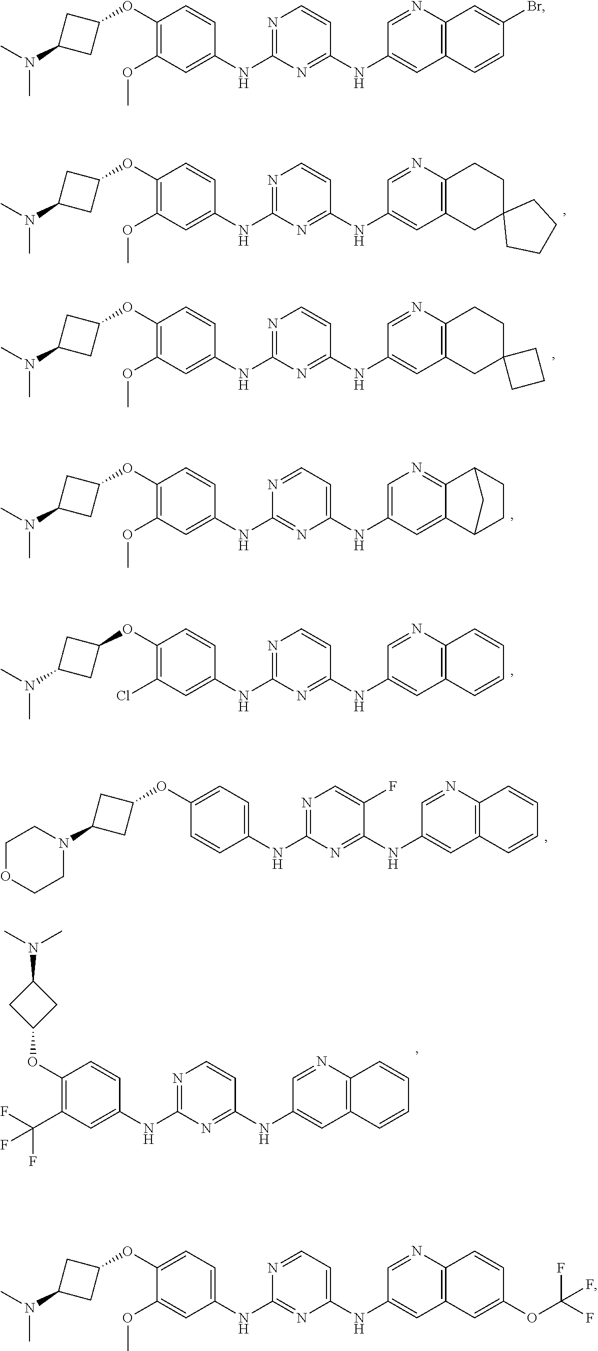

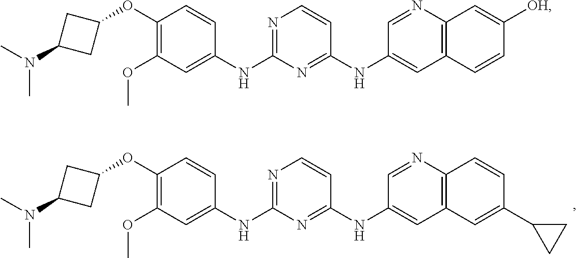 [SMILES: COc1cc(Nc2nccc(Nc3cnc4cc(O)ccc4c3)n2)ccc1O[C@H]1C[C@H](N(C)C)C1 ; COc1cc(Nc2nccc(Nc3cnc4ccc(C5CC5)cc4c3)n2)ccc1O[C@H]1C[C@H](N(C)C)C1]

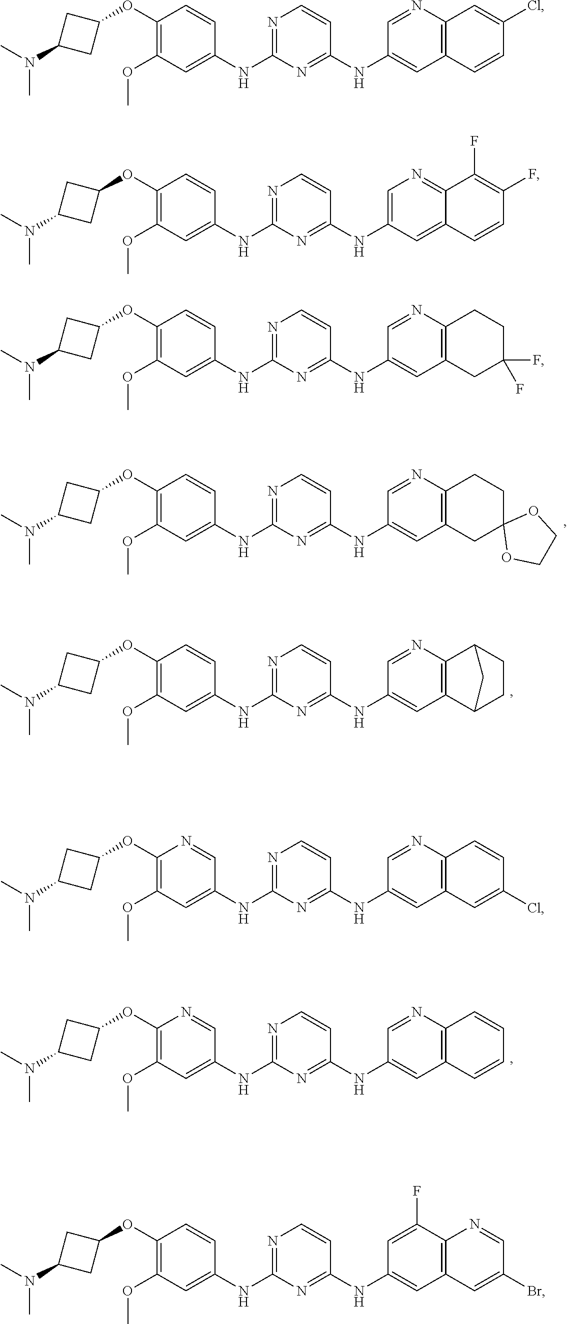

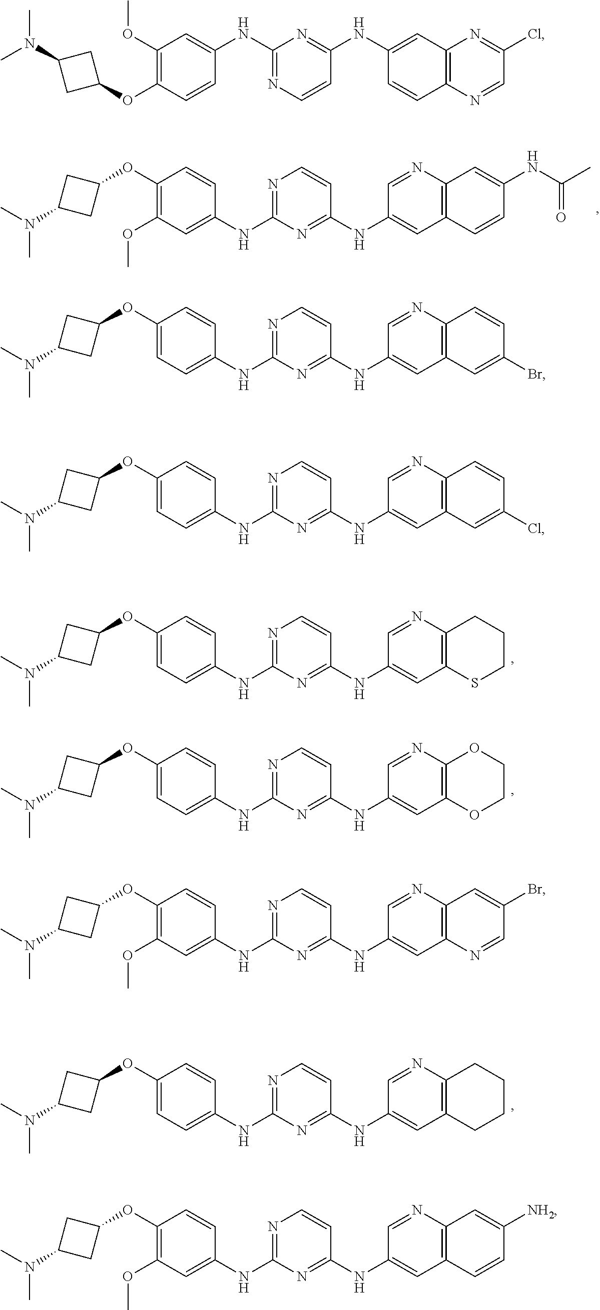

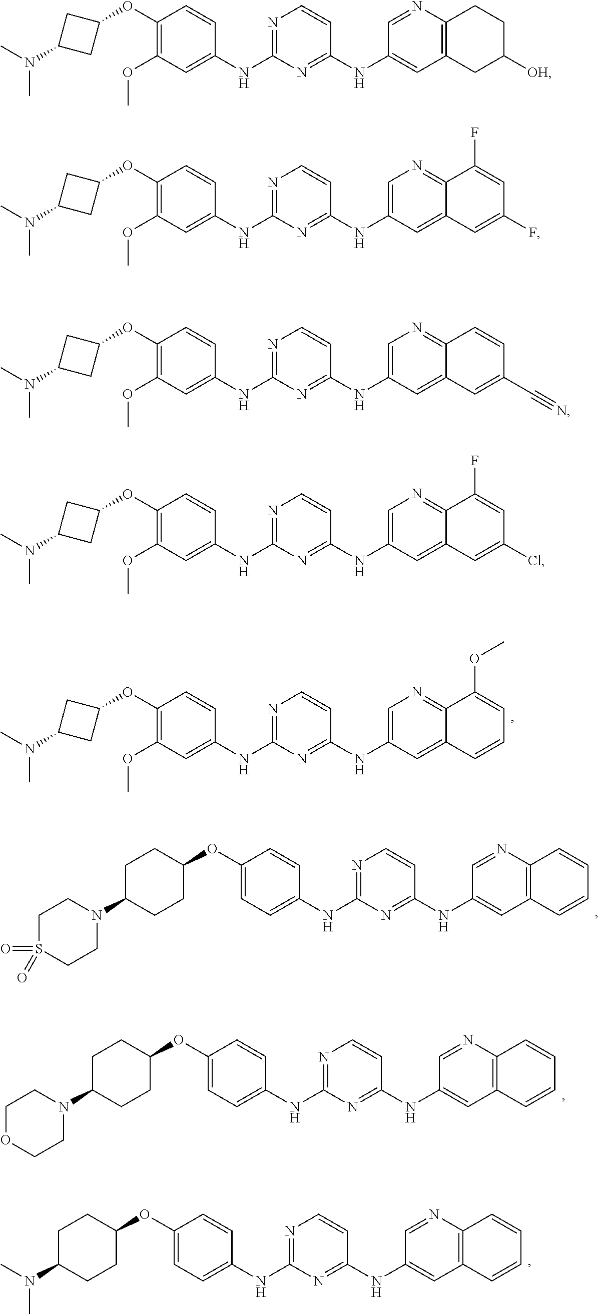

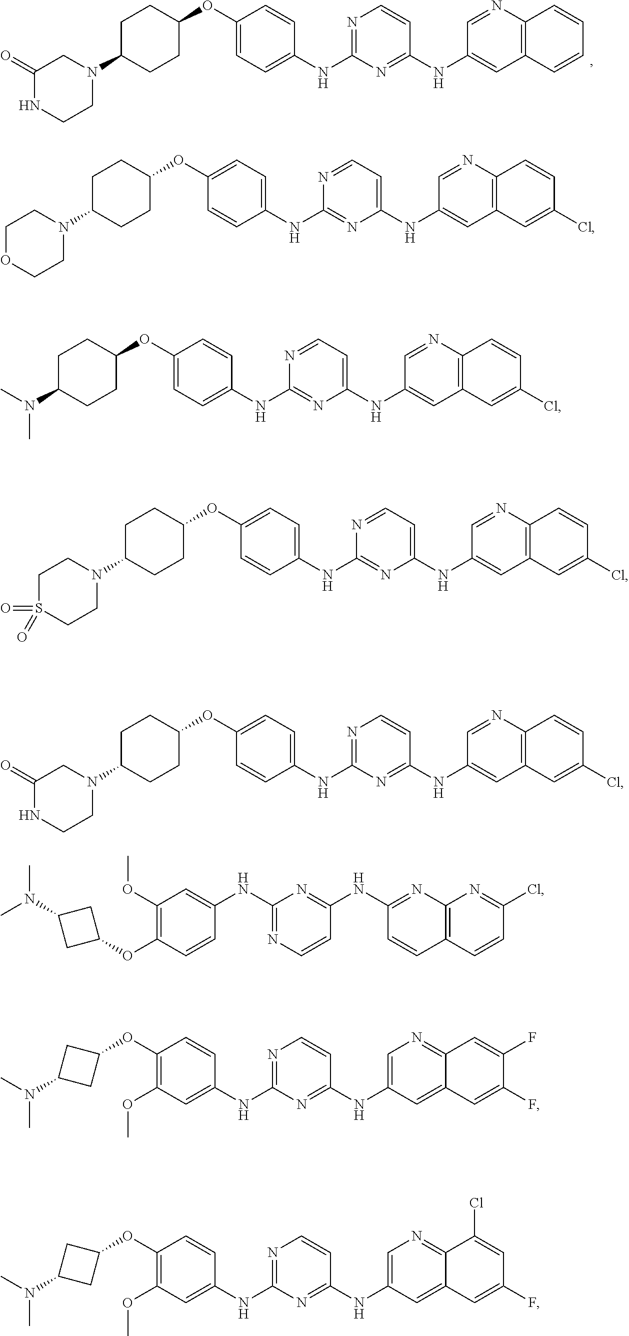

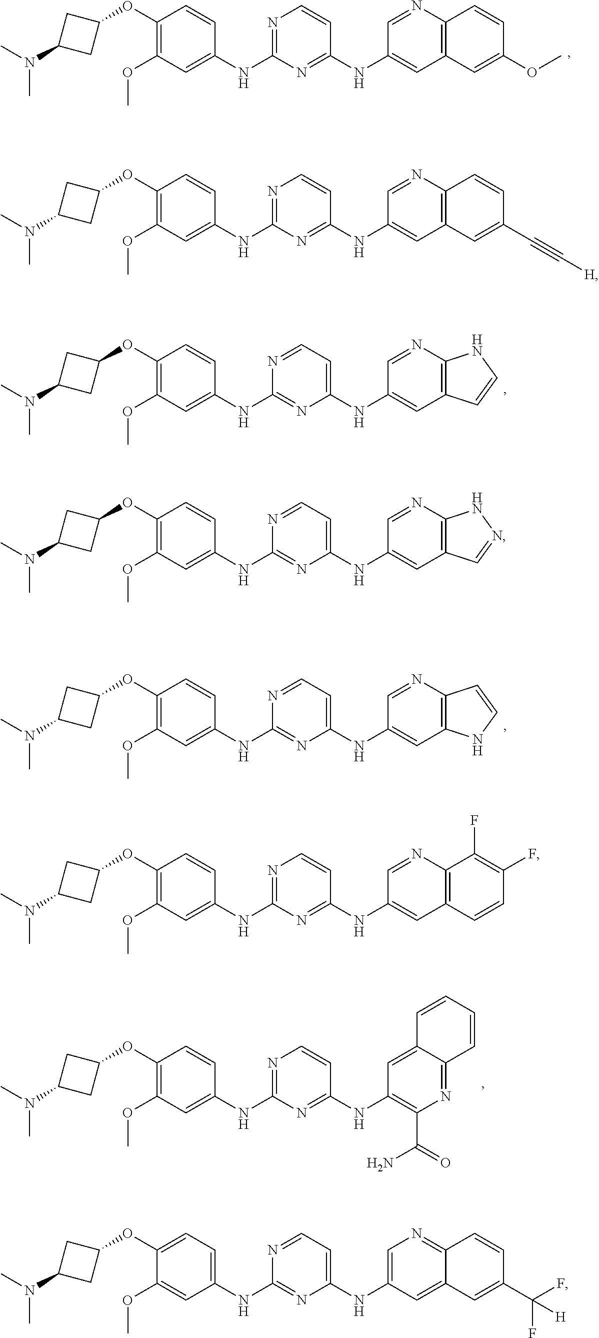

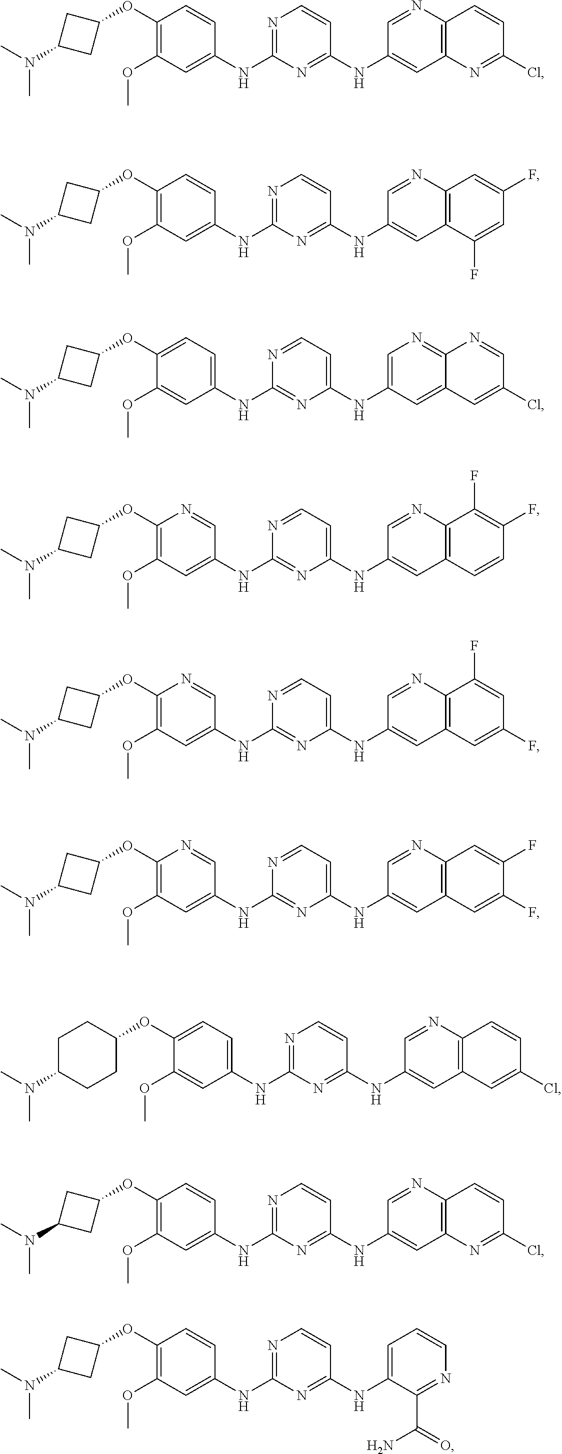

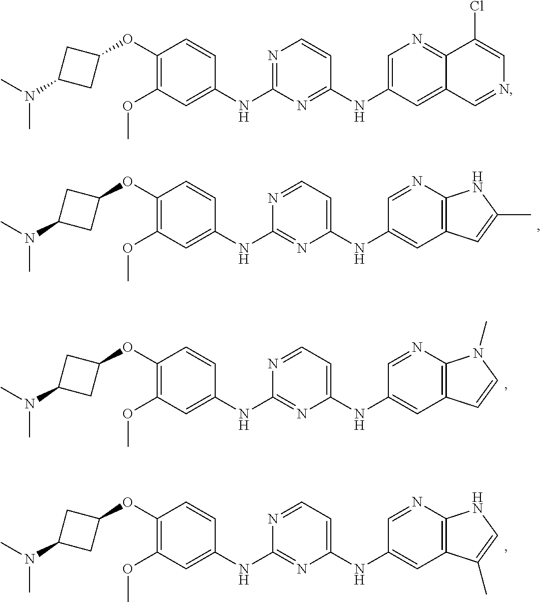

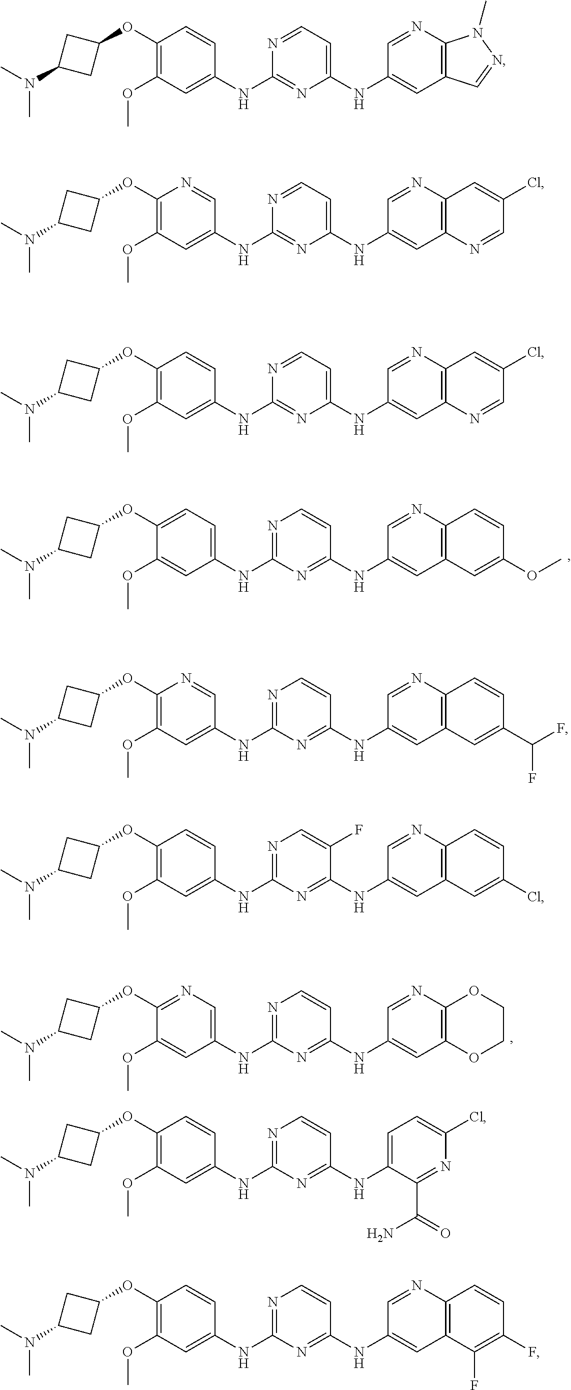

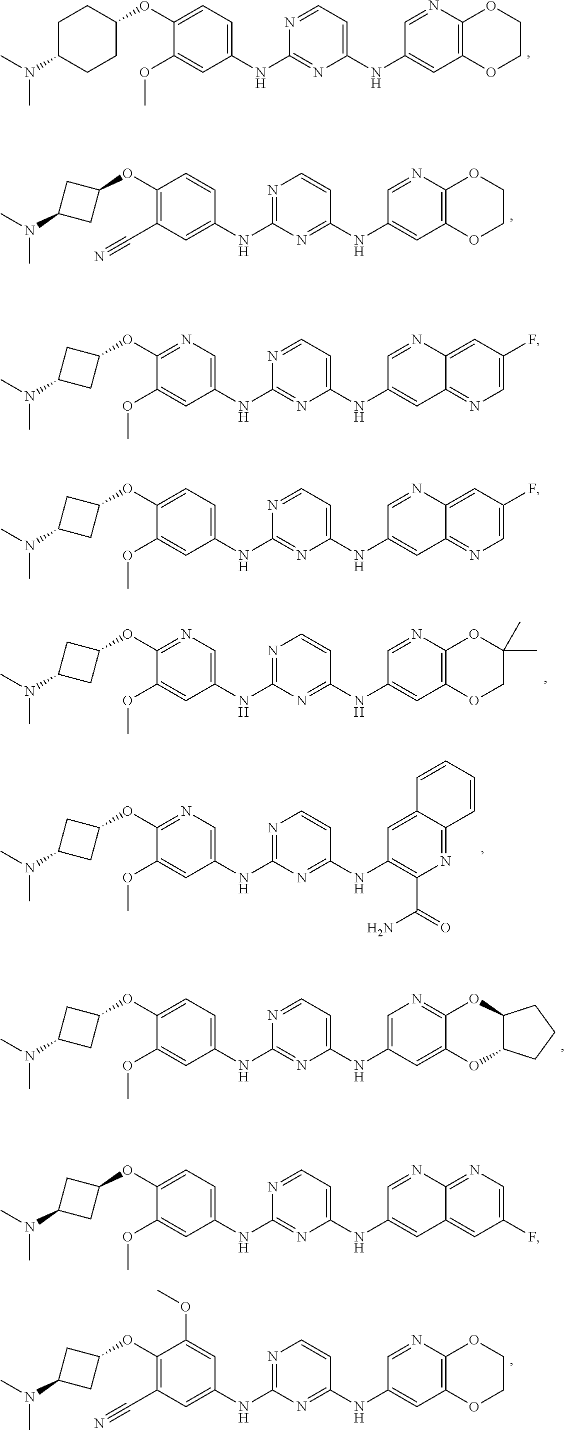

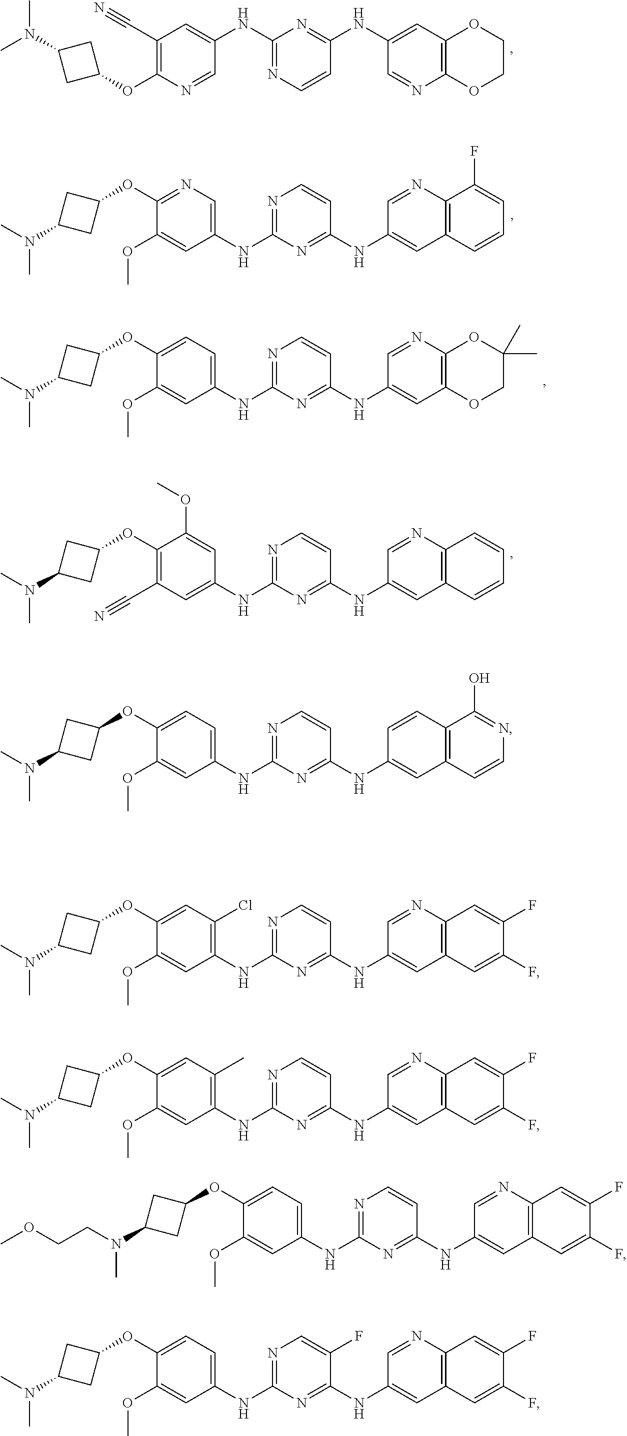

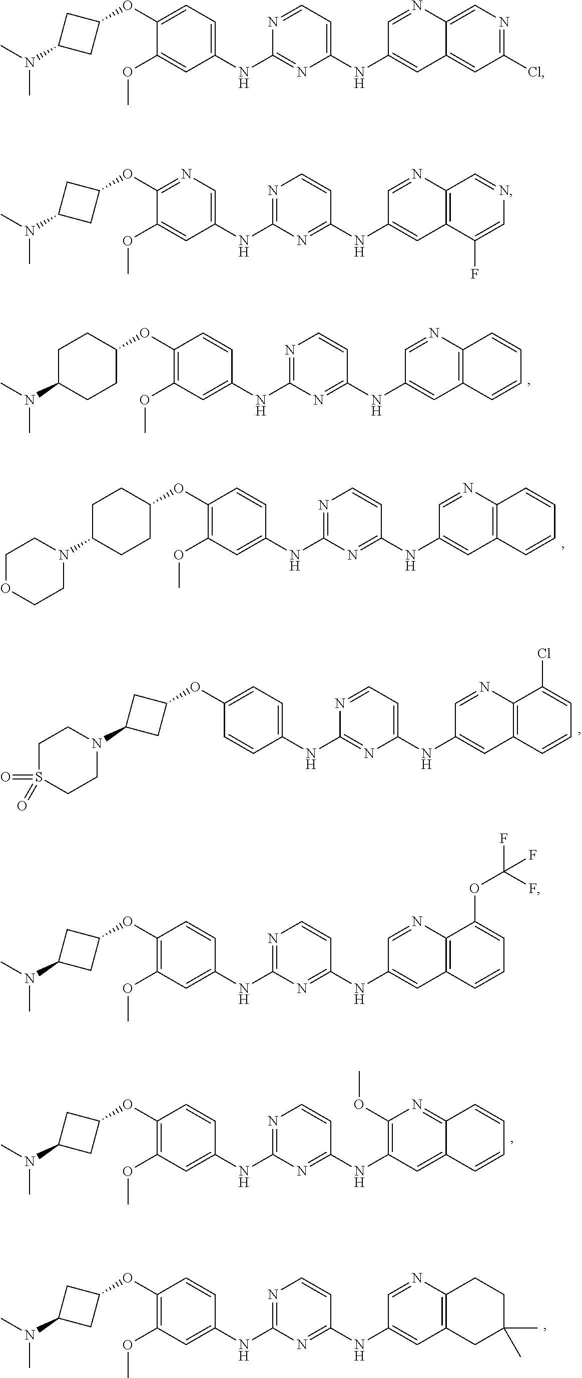

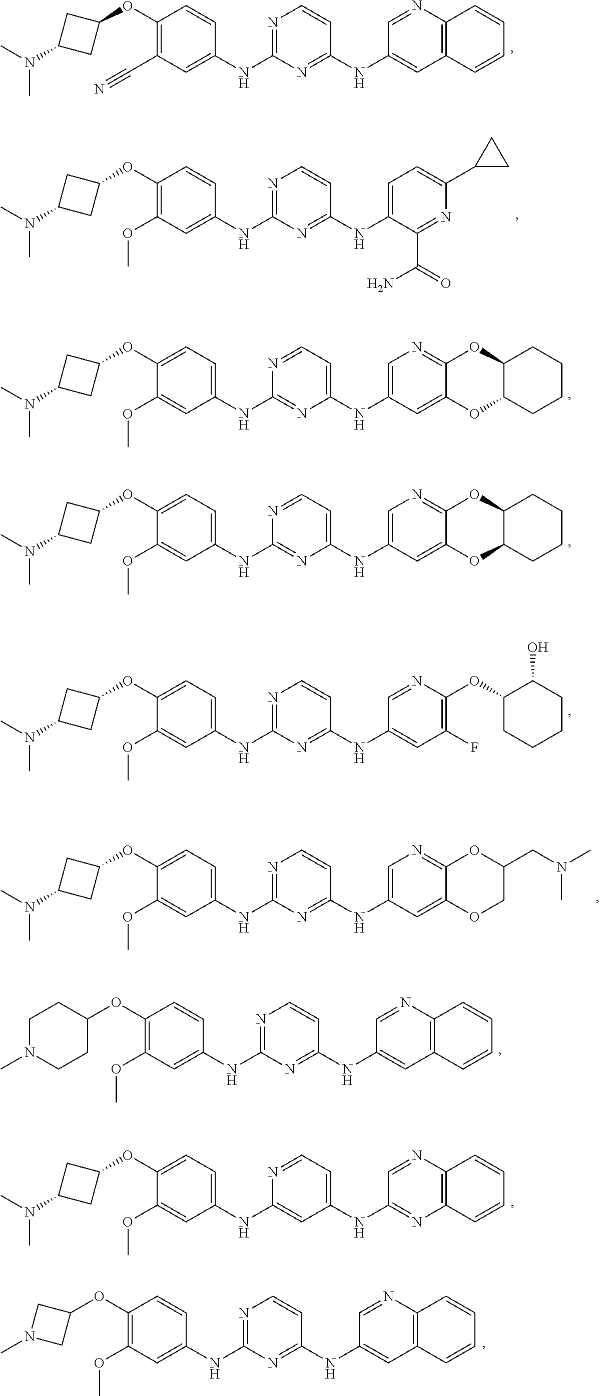

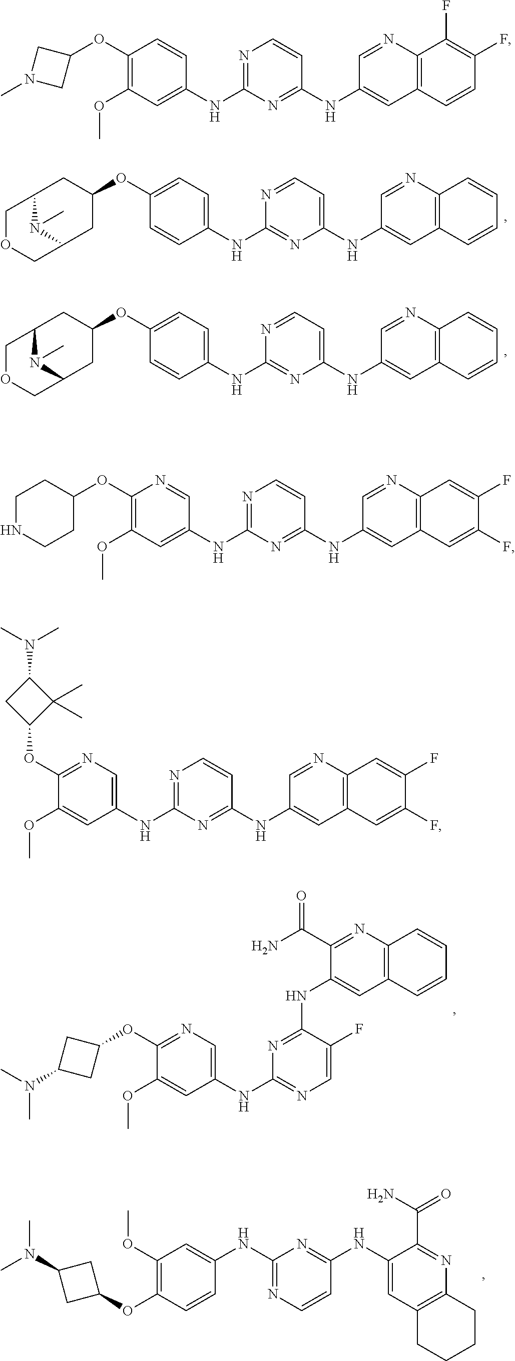

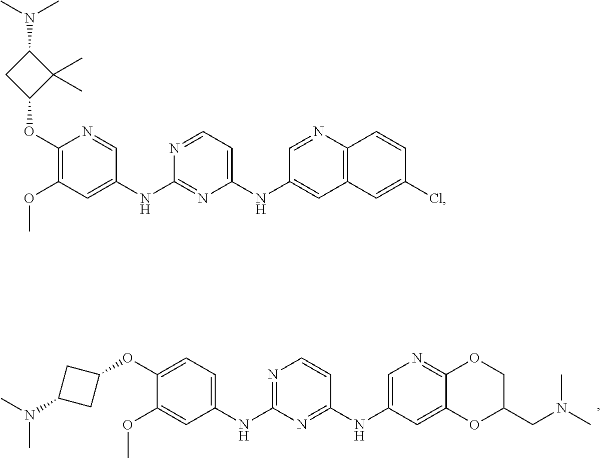

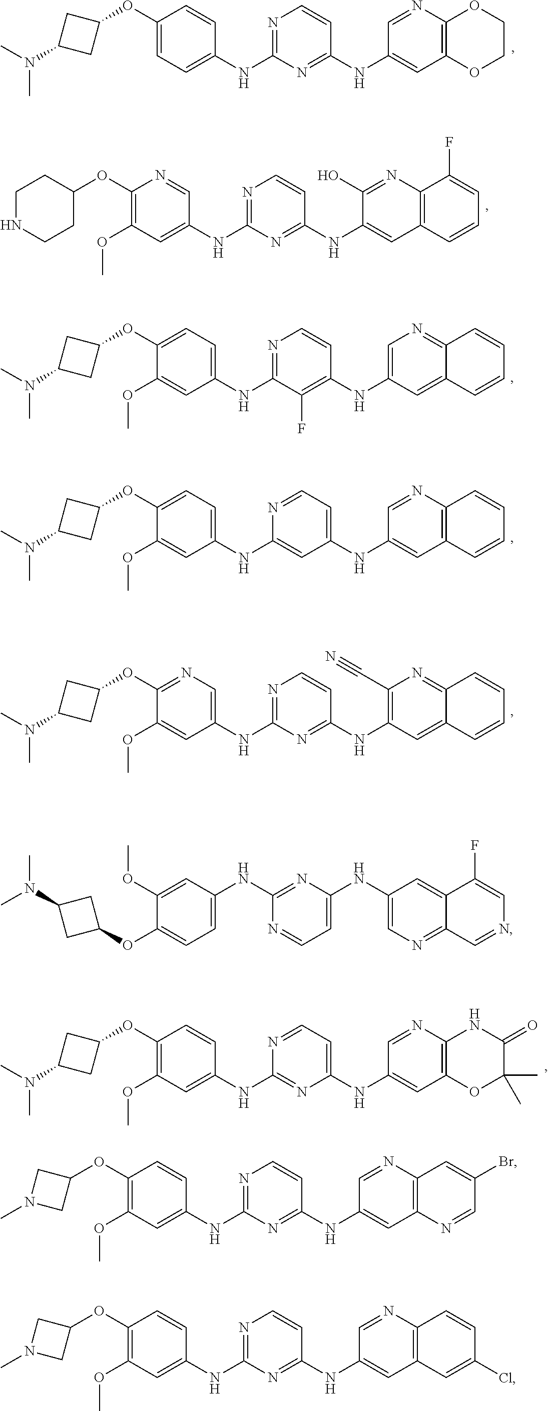

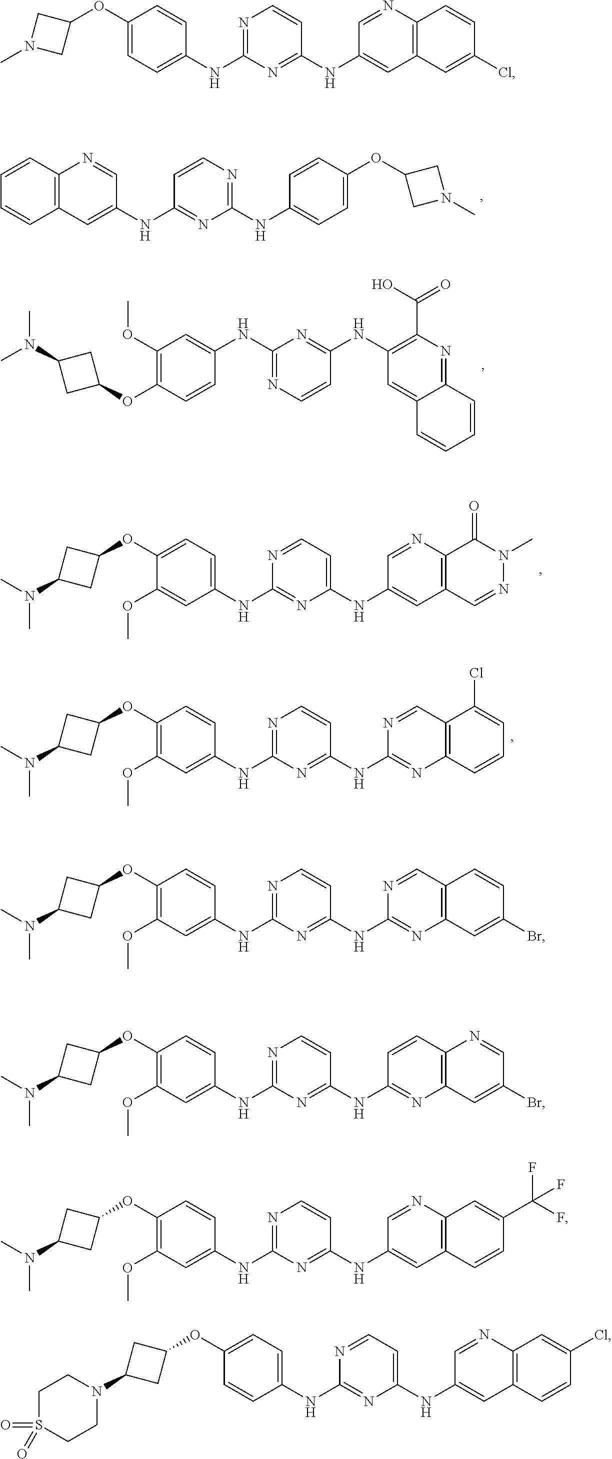

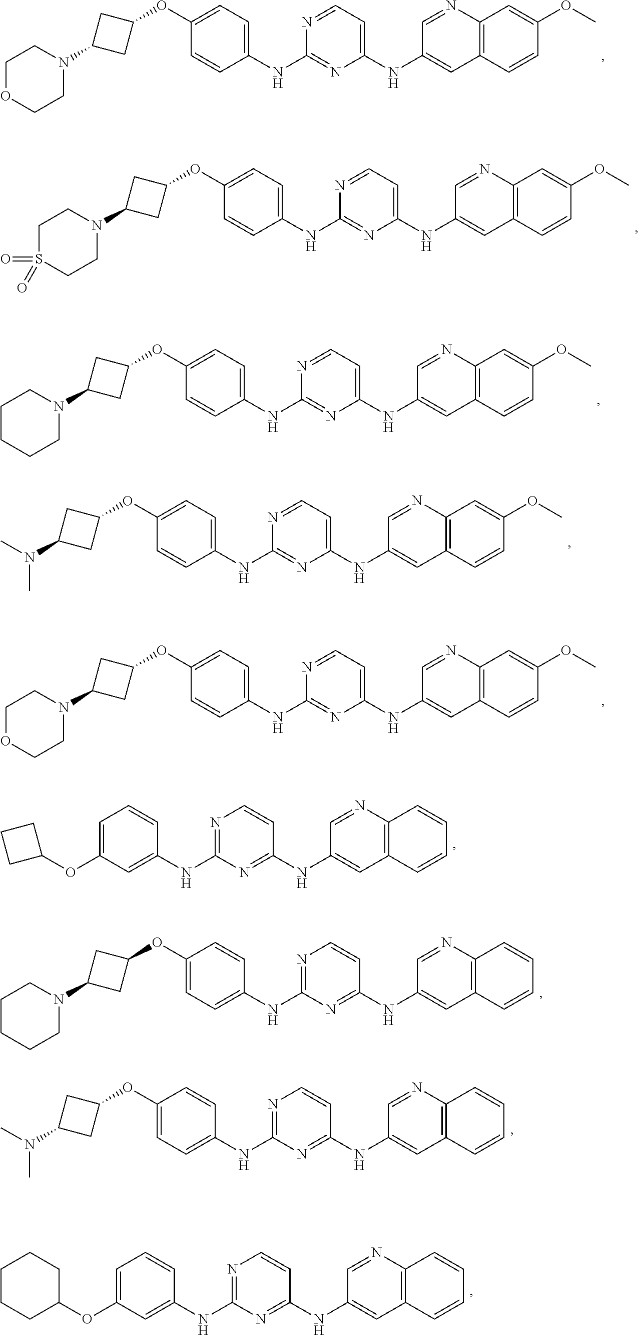

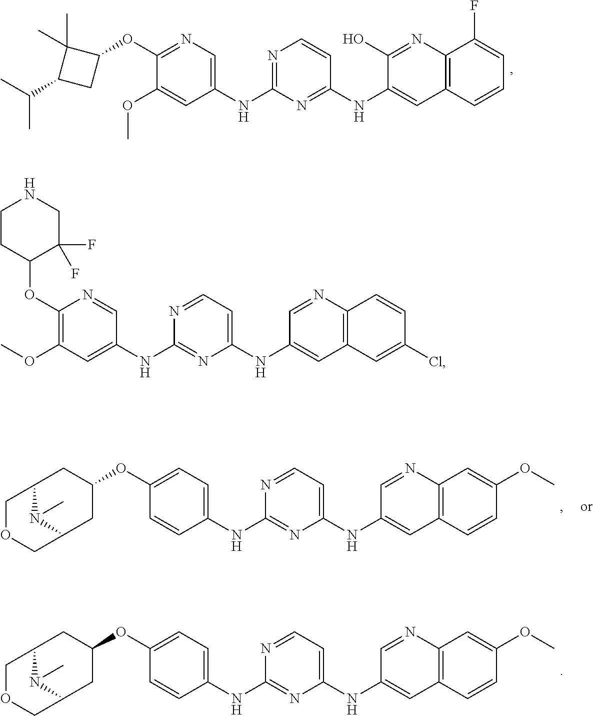 [SMILES: COc1cc(Nc2nccc(Nc3cc4cccc(F)c4nc3O)n2)cnc1O[C@@H]1C[C@H](C(C)C)C1(C)C ; COc1cc(Nc2nccc(Nc3cnc4ccc(Cl)cc4c3)n2)cnc1OC1CCNCC1(F)F ; COc1ccc2cc(Nc3ccnc(Nc4ccc(O[C@@H]5C[C@H]6COC[C@@H](C5)N6C)cc4)n3)cnc2c1 ; COc1ccc2cc(Nc3ccnc(Nc4ccc(O[C@H]5C[C@H]6COC[C@@H](C5)N6C)cc4)n3)cnc2c1]

35. A compound that has the structure of Formula (IIIA1), or a pharmaceutically acceptable salt thereof: 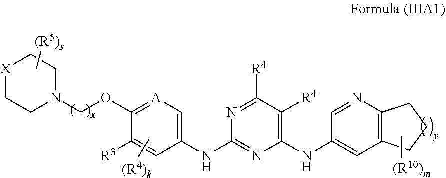
wherein:
x is 1, 2, 3, 4, 5, or 6;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR¹⁹, —C(═O)R²¹, —CO₂R²¹, —N(R²¹)₂, —SR²¹, —S(═O)R²⁰, or —SO₂R²¹;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
y is 1, 2, or 3;
each R¹⁰ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —CO₂R²¹, —C(═O)N(R²¹)₂, —N(R²¹)₂, —NR²¹C(═O)R²¹, —SR²⁰, —S(═O)R²⁰, —SO₂R²⁰, or —SO₂N(R²¹);
m is 0, 1, 2, 3, or 4;
wherein two R¹⁰ can be optionally connected by L;
L is C₁-C₆ alkyl, —N—, or —O—;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

36. The compound of claim 35, or a pharmaceutically acceptable salt thereof, wherein:
x is 1, 2, 3, or 4;
R³ is hydrogen, halogen, unsubstituted or substituted C₁-C₆alkyl, or —O(unsubstituted or substituted C₁-C₆alkyl);
X is —C(R⁸)₂—, —O—, or —SO₂—; and
each R⁸ is independently hydrogen, halogen, unsubstituted or substituted C₁-C₆alkyl.

37. The compound of claim 35, or a pharmaceutically acceptable salt thereof, wherein: 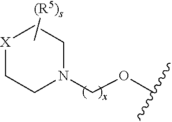
is

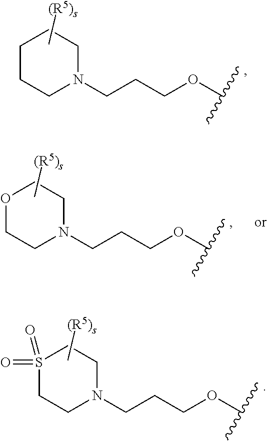

38. The compound of claim 35, or a pharmaceutically acceptable salt thereof, wherein:
y is 1 or 2;
each R¹⁰ is independently hydrogen, halogen, unsubstituted or substituted C₁-C₆alkyl, unsubstituted or substituted C₁-C₆fluoroalkyl, —OH, —OR²⁰, or —N(R²¹)₂;
m is 0, 1, 2, 3, or 4;
wherein two R¹⁰ can be optionally connected by L; and
L is C₁-C₆ alkyl or —O—.

39. The compound of claim 35, or a pharmaceutically acceptable salt thereof, wherein:
y is 1 or 2;
each R¹⁰ is independently hydrogen, halogen, or unsubstituted or substituted C₁-C₆alkyl;
m is 0, 1, 2, 3, or 4;
wherein two R¹⁰ can be optionally connected by L; and
L is unsubstituted or substituted C₁-C₆ alkyl or —O—.

40. The compound of claim 35, or a pharmaceutically acceptable salt thereof, wherein: 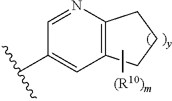
is

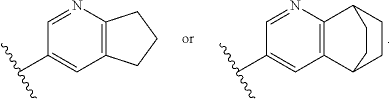 [SMILES: *c1cnc2c(c1)CCC2 ; *c1cnc2c(c1)C1CCC2CC1]

41. The compound of claim 35, wherein the compound has one of the following structures, or a pharmaceutically acceptable salt thereof:

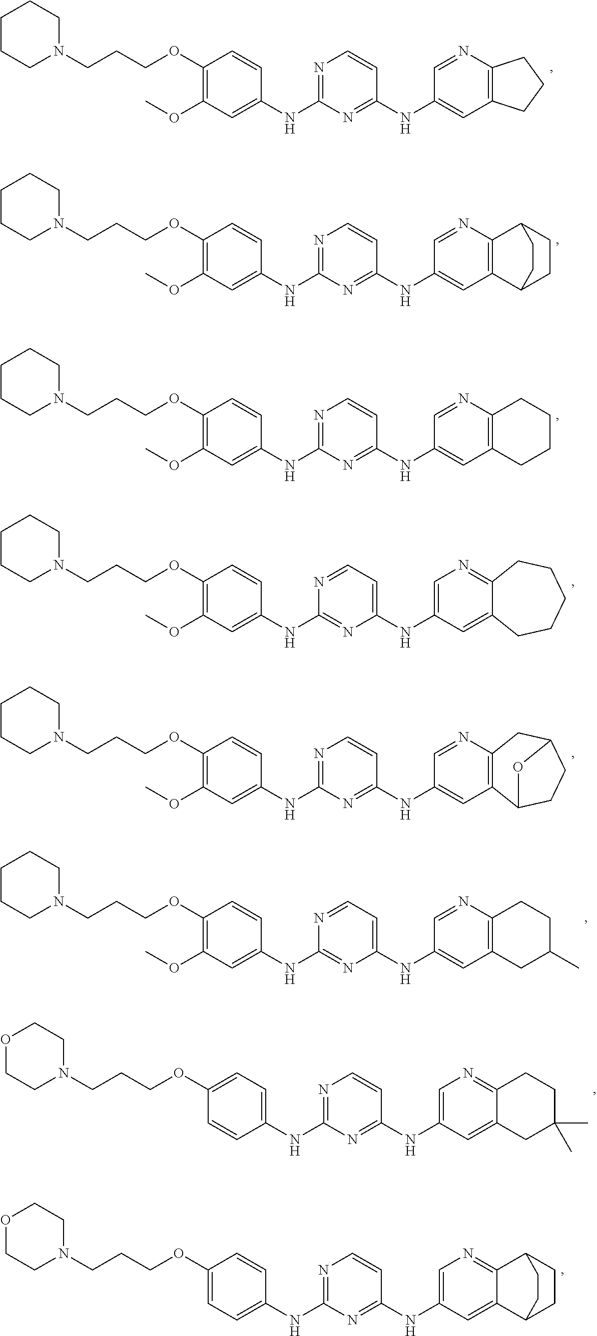

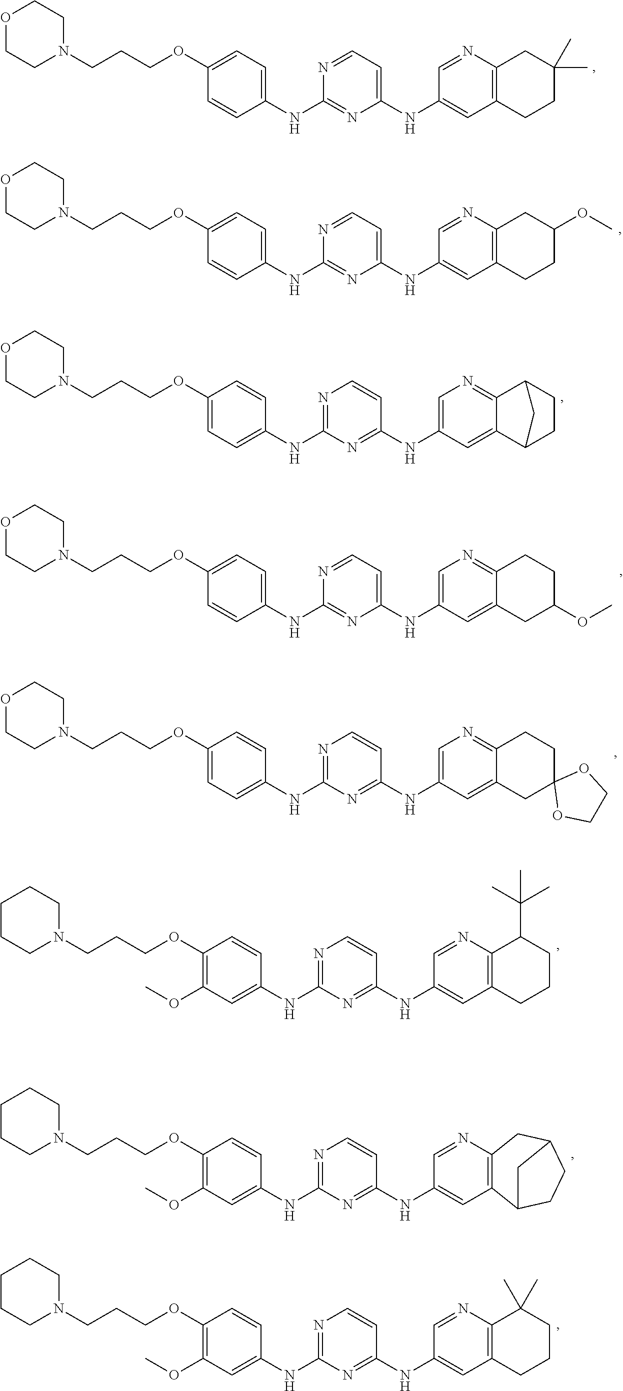

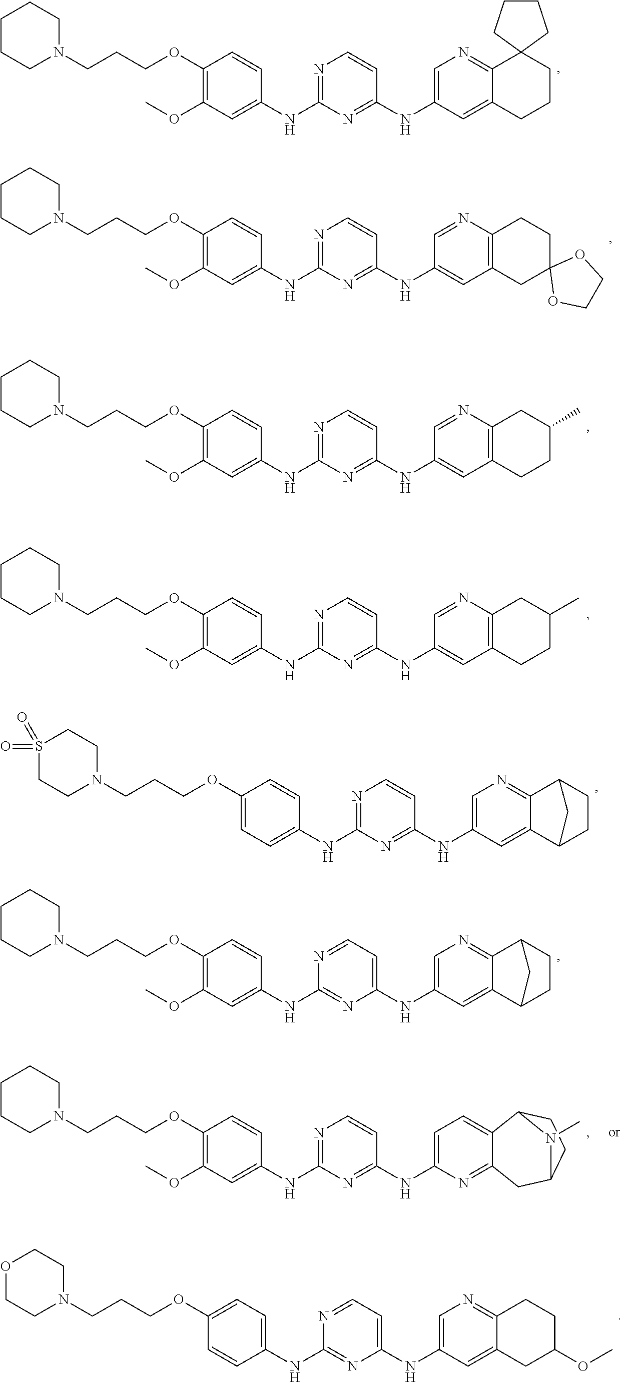

42. A compound that has the structure of Formula (IIIA2), or a pharmaceutically acceptable salt thereof: 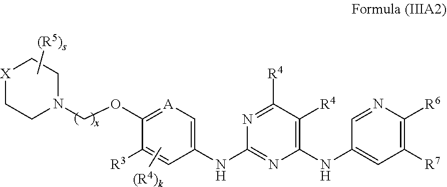
wherein:
x is 1, 2, 3, 4, 5, or 6;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²¹;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
R⁶ and R⁷ are taken together with the intervening carbon atoms connecting R⁶ to R⁷ to form 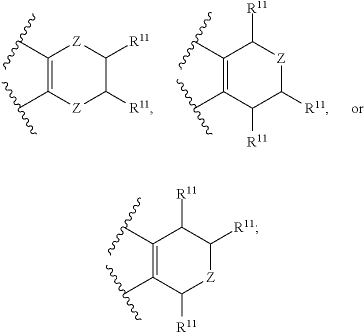
each Z is independently —C(R¹¹)₂, —N(R¹²)—, —O—, —S—, or —SO₂—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

43. The compound of claim 42, or a pharmaceutically acceptable salt thereof, wherein:
x is 1, 2, 3, or 4;
R³ is hydrogen, halogen, unsubstituted or substituted C₁-C₆alkyl, or —O(unsubstituted or substituted C₁-C₆alkyl);
X is —C(R⁸)₂—, —O—, or —SO₂—; and
each R⁸ is independently hydrogen, halogen, unsubstituted or substituted C₁-C₆alkyl.

44. The compound of claim 42, or a pharmaceutically acceptable salt thereof, wherein: 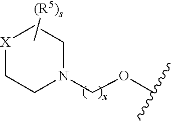
is

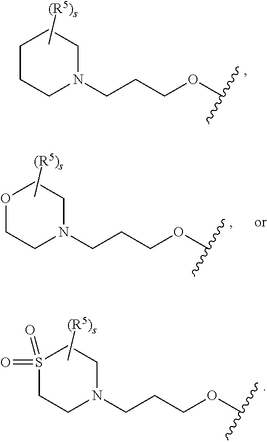

45. The compound of claim 42, or a pharmaceutically acceptable salt thereof, wherein:
R⁶ and R⁷ are taken together with the intervening carbon atoms connecting R⁶ to R⁷ to form 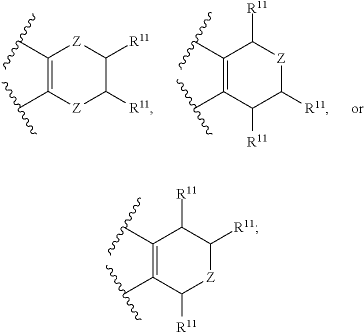
each Z is independently —C(R¹¹)₂, —N(R¹²)—, —O—, —S—, or —SO₂—;
each R¹¹ is independently hydrogen, halogen, unsubstituted or substituted C₁-C₆alkyl, unsubstituted or substituted C₁-C₆fluoroalkyl, oxo, —CN, —OH, —OR²⁰, or —N(R²¹)₂; and
each R¹² is hydrogen or unsubstituted or substituted C₁-C₆alkyl.

46. The compound of claim 42, or a pharmaceutically acceptable salt thereof, wherein:
R⁶ and R⁷ are taken together with the intervening carbon atoms connecting R⁶ to R⁷ to form

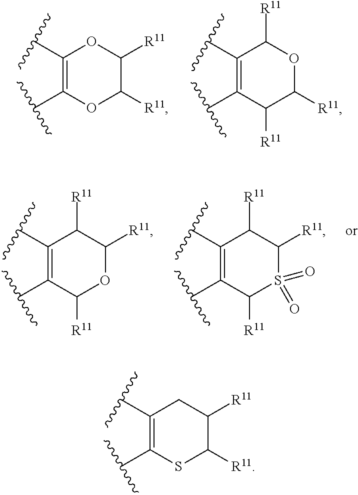

47. The compound of claim 42, or a pharmaceutically acceptable salt thereof, wherein:
each R¹¹ is independently hydrogen or methyl.

48. The compound of claim 42, wherein the compound has one of the following structures, or a pharmaceutically acceptable salt thereof:

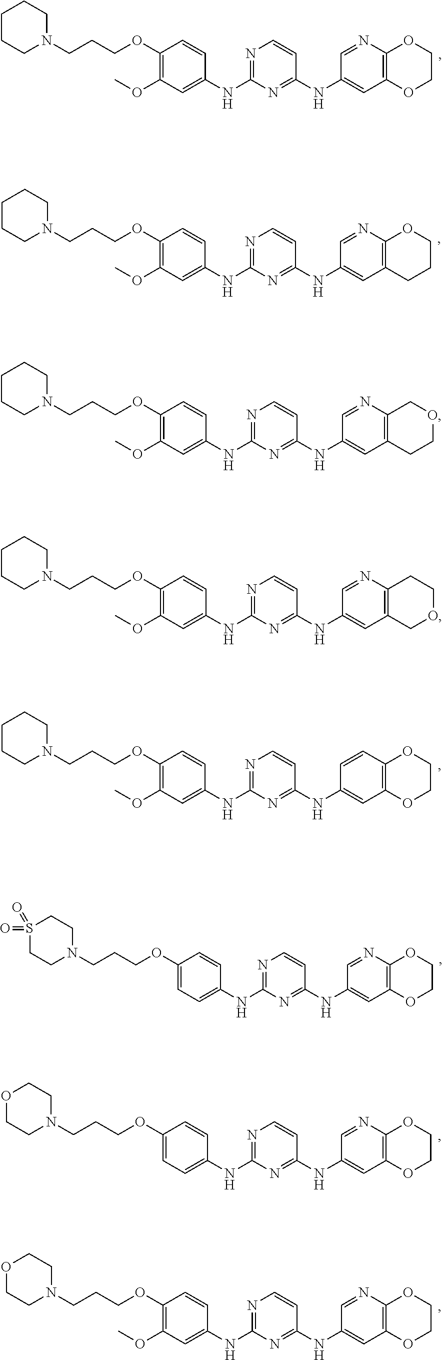

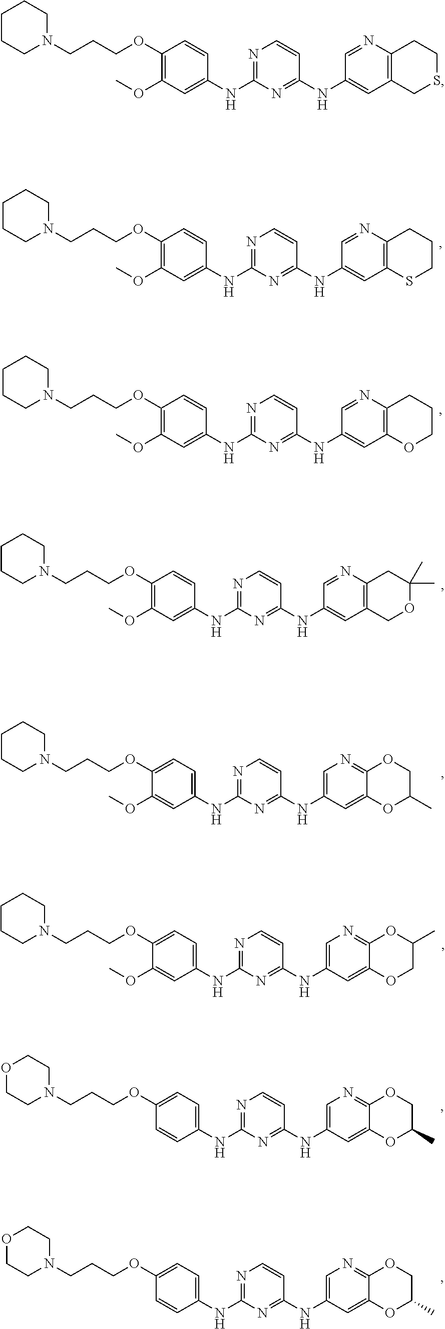

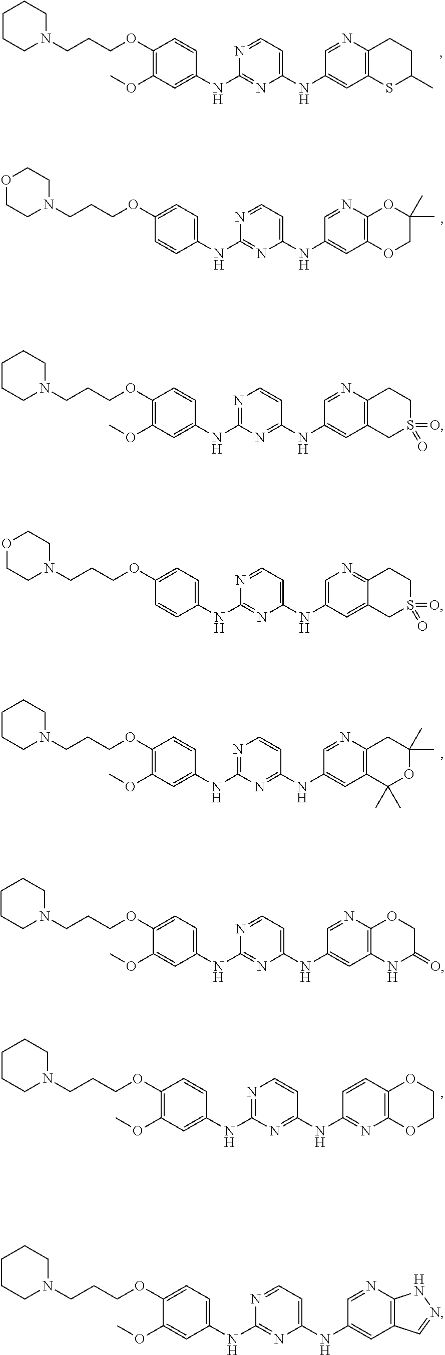

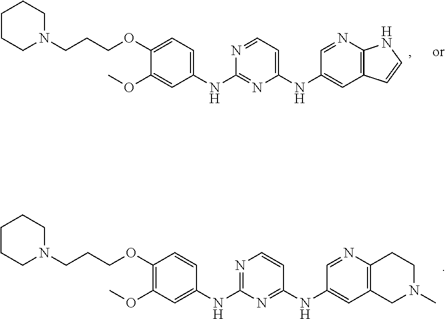 [SMILES: COc1cc(Nc2nccc(Nc3cnc4[nH]ccc4c3)n2)ccc1OCCCN1CCCCC1 ; COc1cc(Nc2nccc(Nc3cnc4c(c3)CN(C)CC4)n2)ccc1OCCCN1CCCCC1]

49. A compound that has the structure of Formula (IIIB), or a pharmaceutically acceptable salt thereof: 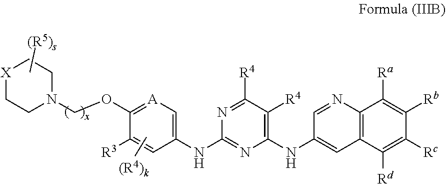
wherein:
x is 1, 2, 3, 4, 5, or 6;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²¹;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
R^{a} is hydrogen, halogen, C₁-C₆alkyl, C₁-C₆fluoroalkyl, C₁-C₆heteroalkyl, C₃-C₈cycloalkyl, C₃-C₈heterocycloalkyl, —CN, —OH, —OR²⁰, or —N(R²¹)₂;
R^{b} is hydrogen, halogen, C₁-C₆alkyl, C₃-C₈cycloalkyl, C₃-C₈heterocycloalkyl, —CN, —OH, —OR²⁰ or —N(R²¹)₂;
R^{c} is hydrogen, halogen, C₁-C₆alkyl, C₁-C₆fluoroalkyl, C₃-C₈cycloalkyl, C₃-C₈heterocycloalkyl, —CN, —OH, or —N(R²¹)₂;
R^{d} is hydrogen, halogen, C₁-C₆alkyl, C₁-C₆fluoroalkyl, C₁-C₆heteroalkyl, C₃-C₈cycloalkyl, C₃-C₈heterocycloalkyl, —CN, —OH, —OR²⁰, or —N(R²¹)₂;
wherein at least one of R^{a}, R^{b}, R^{c}, and R^{d} is not hydrogen;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

50. The compound of claim 49, or a pharmaceutically acceptable salt thereof, wherein:
x is 1, 2, 3, or 4;
R³ is hydrogen, halogen, unsubstituted or substituted C₁-C₆alkyl, or —O(unsubstituted or substituted C₁-C₆alkyl);
X is —C(R⁸)₂—, —O—, or —SO₂—; and
each R⁸ is independently hydrogen, halogen, unsubstituted or substituted C₁-C₆alkyl.

51. The compound of claim 48, or a pharmaceutically acceptable salt thereof, wherein: 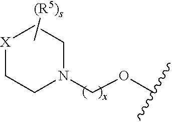
is

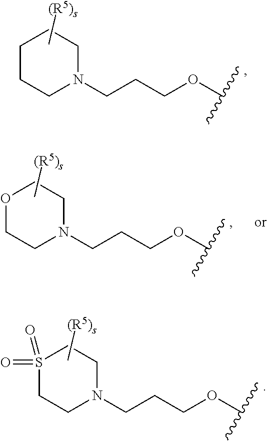

52. The compound of claim 49, or a pharmaceutically acceptable salt thereof, wherein:
R^{a} is hydrogen, halogen, unsubstituted or substituted C₁-C₆alkyl, unsubstituted or substituted C₁-C₆fluoroalkyl, or —OR²⁰;
R^{b} is hydrogen, halogen, unsubstituted or substituted C₁-C₆alkyl, unsubstituted or substituted C₃-C₈cycloalkyl, —OR²⁰, or —N(R²¹)₂;
R^{c} is hydrogen, halogen, unsubstituted or substituted C₁-C₆alkyl, unsubstituted or substituted C₁-C₆fluoroalkyl, or —CN; and
R^{d} is hydrogen, halogen, or unsubstituted or substituted C₁-C₆alkyl.

53. The compound of claim 49, or a pharmaceutically acceptable salt thereof, wherein:
R^{a} is hydrogen, halogen, unsubstituted or substituted C₁-C₆alkyl, —CF₃, —OCH₃, or —OCF₃;
R^{b} is hydrogen, halogen, unsubstituted or substituted C₁-C₆alkyl, unsubstituted or substituted C₃-C₆cycloalkyl, —OCH₃, or —OCF₃;
R^{c} is hydrogen, halogen, unsubstituted or substituted C₁-C₆alkyl, —CF₃, or —CN; and
R^{d} is hydrogen, halogen, or unsubstituted or substituted C₁-C₆alkyl.

54. The compound of claim 49, or a pharmaceutically acceptable salt thereof, wherein:
at least one of R^{a}, R^{b}, R^{c}, or R^{d} is halogen.

55. The compound of claim 49, wherein the compound has one of the following structures, or a pharmaceutically acceptable salt thereof:

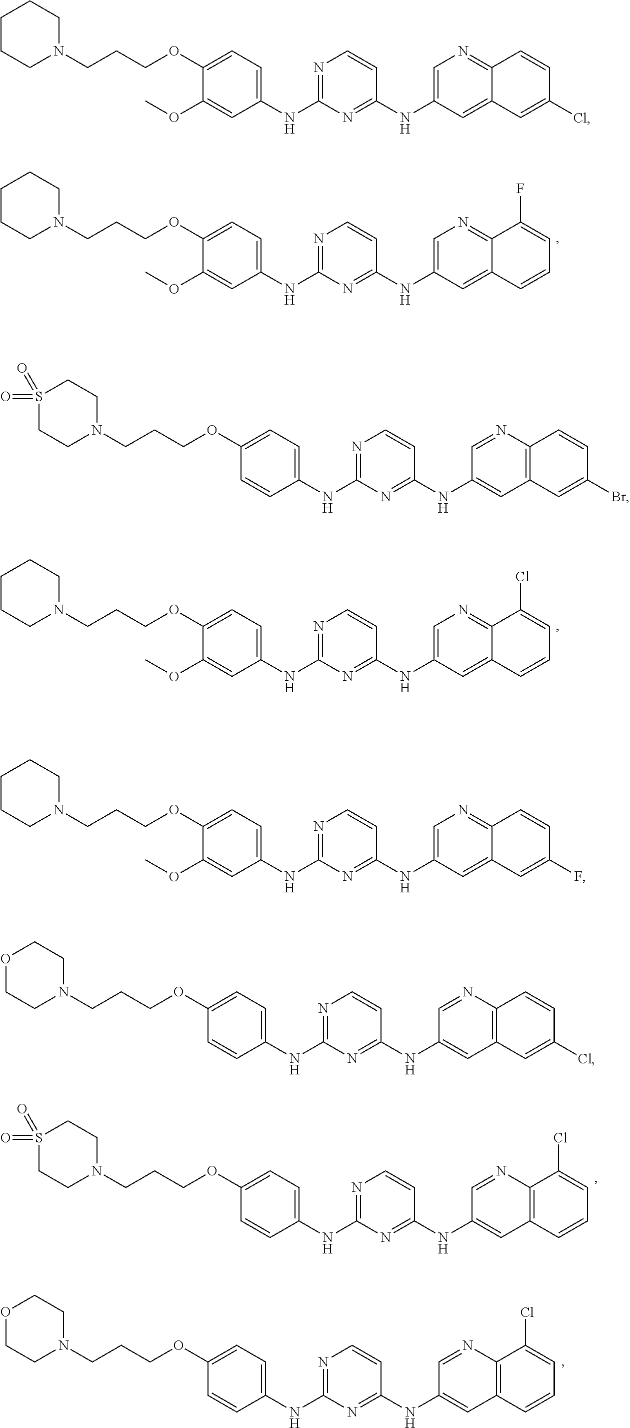

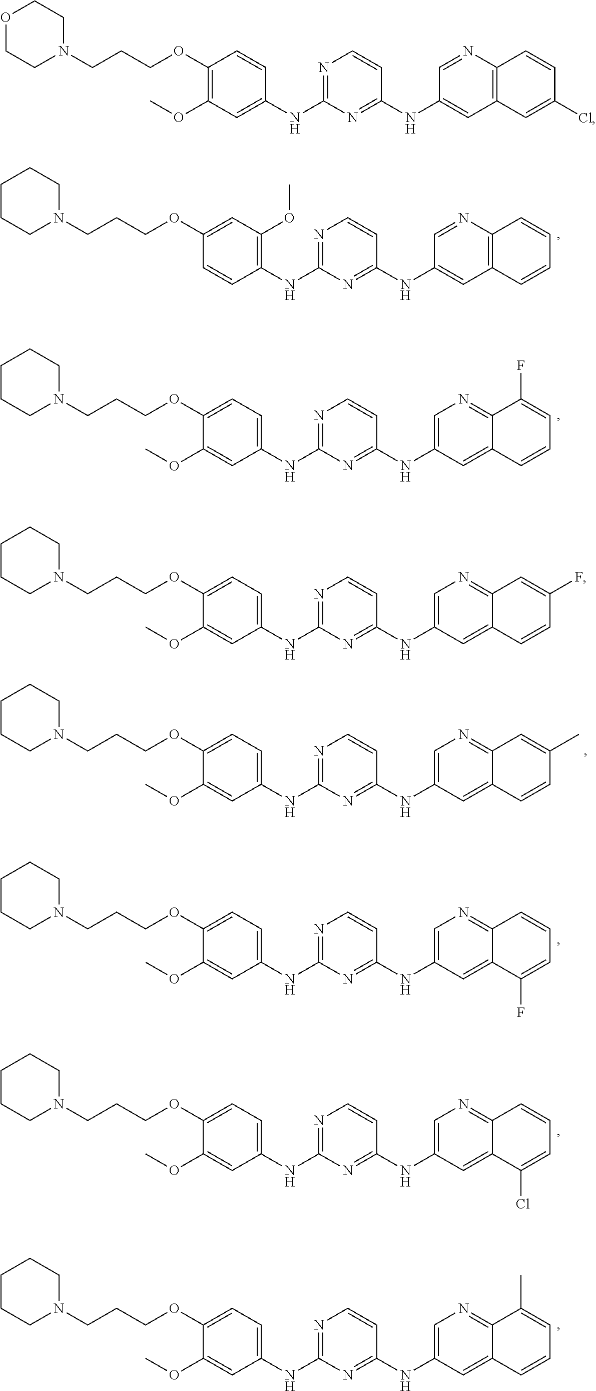

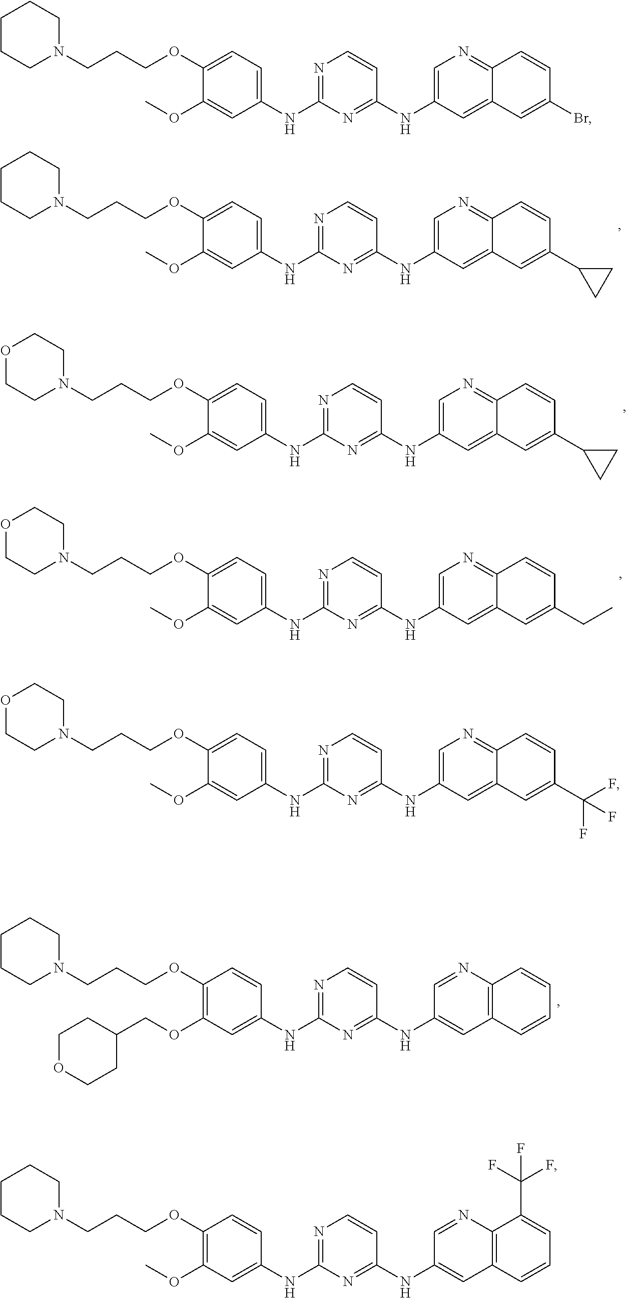

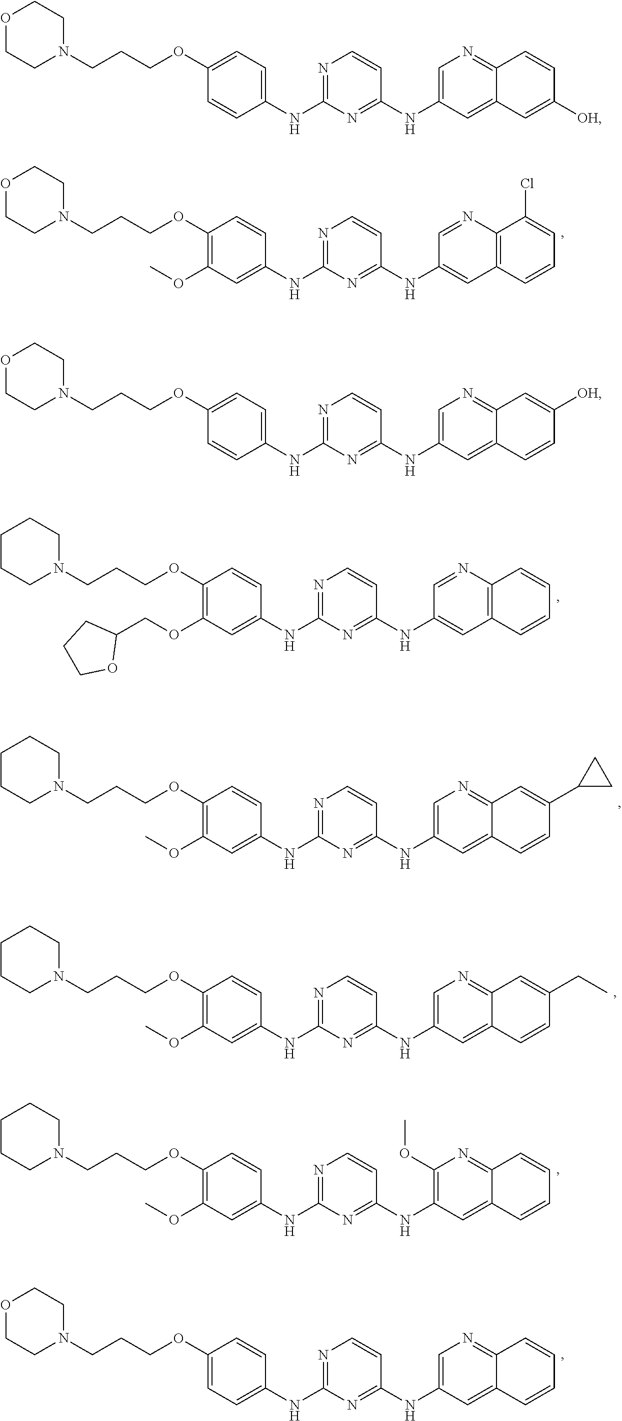

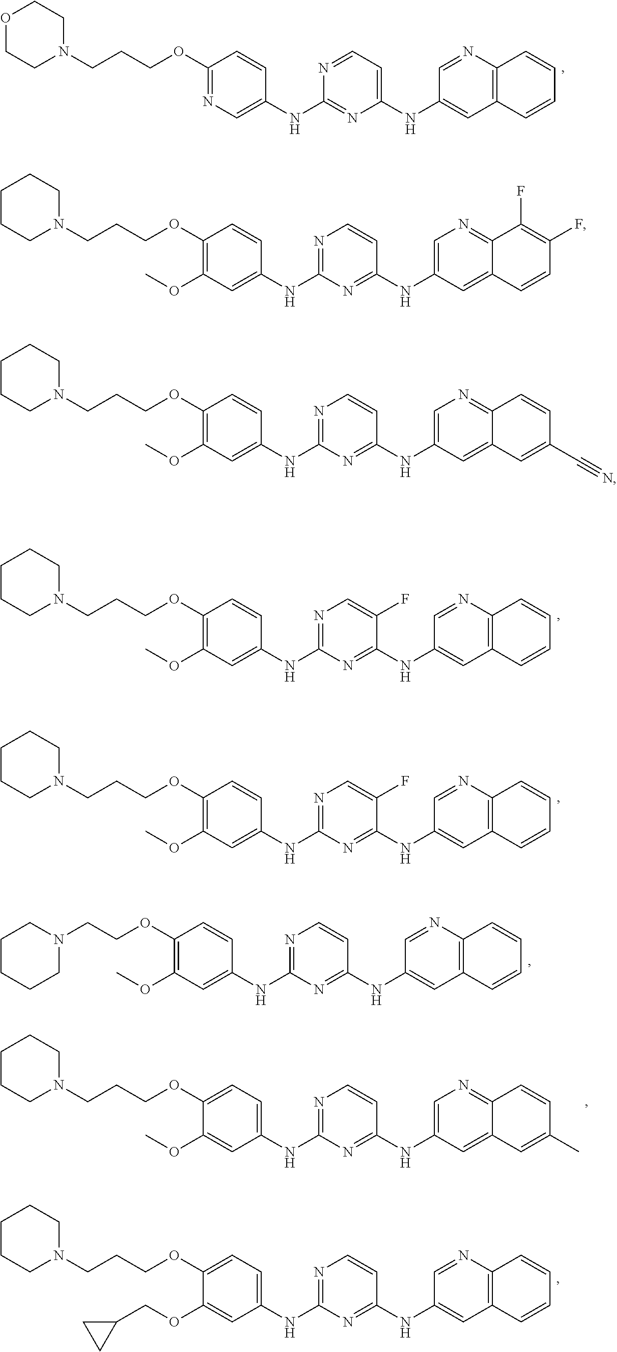

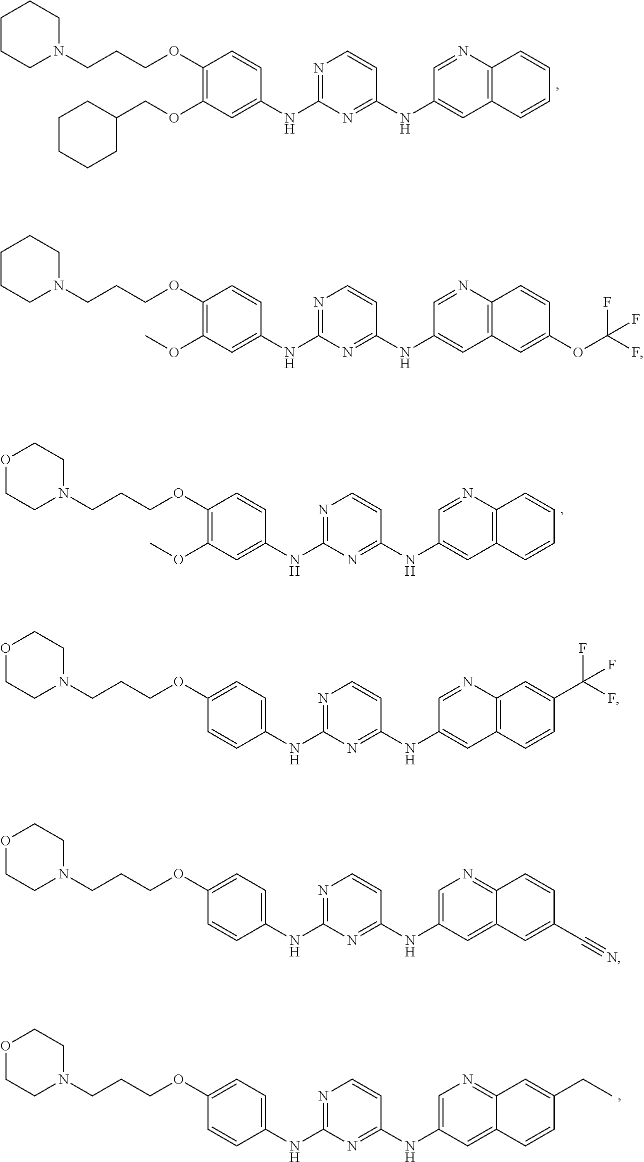 [SMILES: c1ccc2ncc(Nc3ccnc(Nc4ccc(OCCCN5CCCCC5)c(OCC5CCCCC5)c4)n3)cc2c1 ; COc1cc(Nc2nccc(Nc3cnc4ccc(OC(F)(F)F)cc4c3)n2)ccc1OCCCN1CCCCC1 ; COc1cc(Nc2nccc(Nc3cnc4ccccc4c3)n2)ccc1OCCCN1CCOCC1 ; FC(F)(F)c1ccc2cc(Nc3ccnc(Nc4ccc(OCCCN5CCOCC5)cc4)n3)cnc2c1 ; N#Cc1ccc2ncc(Nc3ccnc(Nc4ccc(OCCCN5CCOCC5)cc4)n3)cc2c1 ; CCc1ccc2cc(Nc3ccnc(Nc4ccc(OCCCN5CCOCC5)cc4)n3)cnc2c1]

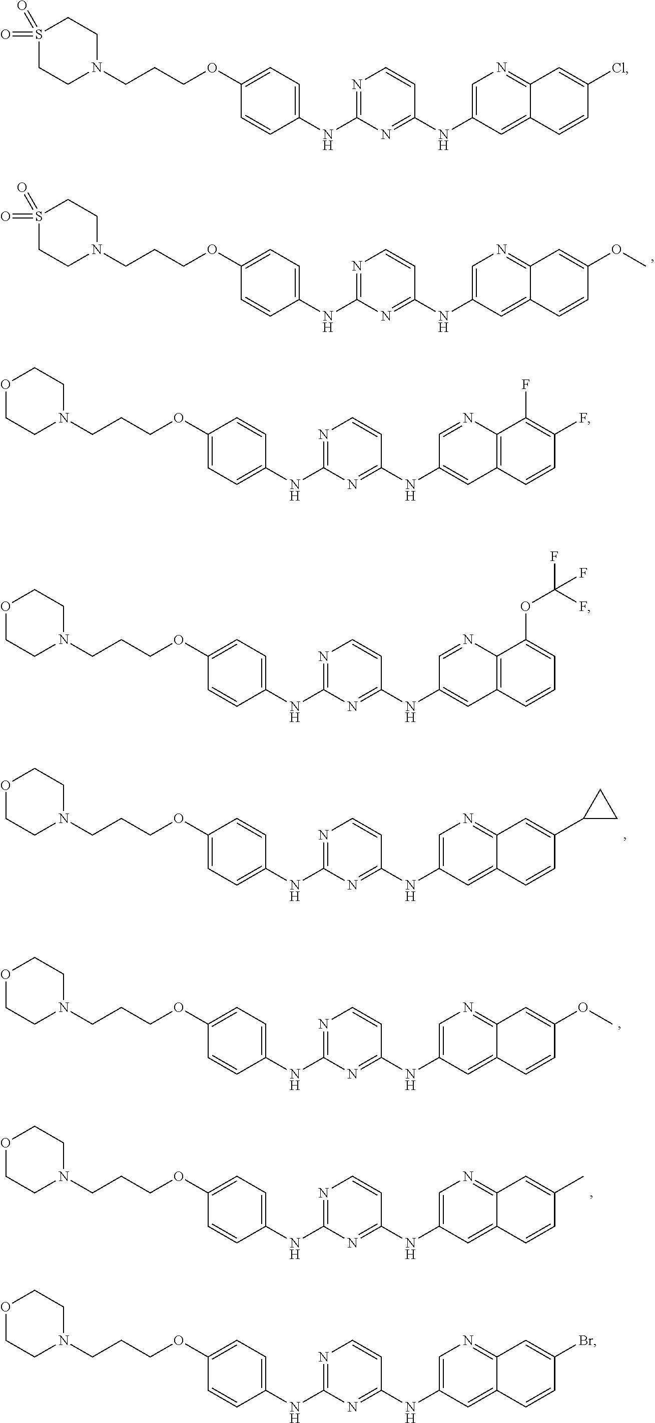

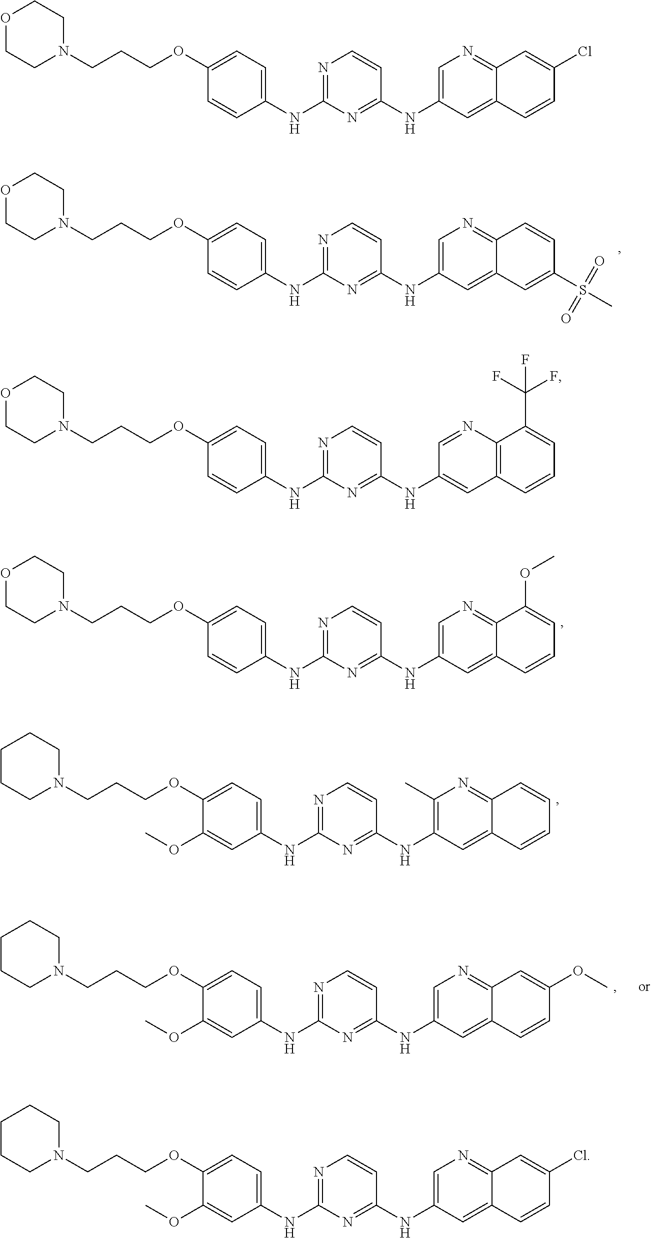

56. A compound that has the structure of Formula (IIIC), or a pharmaceutically acceptable salt thereof: 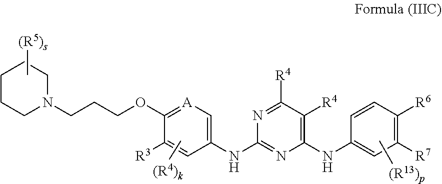
wherein:
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
R³ is —O-(unsubstituted or substituted C₁-C₆alkyl);
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, bicyclic C₅-C₁₀cycloalkyl, bicyclic C₅-C₁₀heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
or R⁶ and R⁷ are taken together with the intervening carbon atoms connecting R⁶ to R⁷ to form 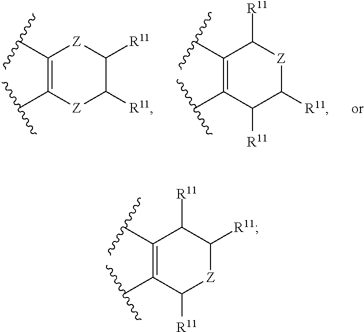
each Z is independently —C(R¹¹)₂, —N(R¹²)—, —O—, or —S—;
each R¹¹ is independently hydrogen, halogen, unsubstituted or substituted C₁-C₆alkyl, unsubstituted or substituted C₁-C₆fluoroalkyl, unsubstituted or substituted C₁-C₆heteroalkyl, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹² is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
each R¹³ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
p is 0, 1, 2, 3, or 4
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

57. The compound of claim 56, or a pharmaceutically acceptable salt thereof, wherein:
s is 0 and k is 0.

58. The compound of claim 56, or a pharmaceutically acceptable salt thereof, wherein:
A is —CH.

59. The compound of claim 56, or a pharmaceutically acceptable salt thereof, wherein:
R³ is —O-(unsubstituted or substituted C₁-C₆alkyl).

60. The compound of claim 56, or a pharmaceutically acceptable salt thereof, wherein:
R³ is —OCH₃.

61. The compound of claim 56, or a pharmaceutically acceptable salt thereof, wherein:
R⁴ is hydrogen.

62. The compound of claim 56, or a pharmaceutically acceptable salt thereof, wherein:
R⁶ and R⁷ are taken together with the intervening carbon atoms connecting R⁶ to R⁷ to form a ring B that is monocyclic C₃-C₈cycloalkyl, monocyclic C₃-C₈heterocycloalkyl, monocyclic C₃-C₈ aryl, or monocyclic C₃-C₈ heteroaryl, wherein ring B is unsubstituted or substituted with 1 or 2 R¹¹, or 0 or 1 R¹²;
or R⁶ and R⁷ are taken together with the intervening carbon atoms connecting R⁶ to R⁷ to form 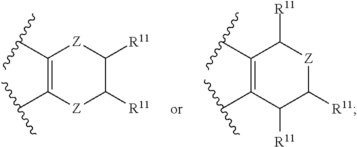
each Z is independently —C(R¹¹)₂, —N(R¹²)—, or —O—;
each R¹¹ is independently hydrogen, halogen, unsubstituted or substituted C₁-C₆alkyl, unsubstituted or substituted C₁-C₆fluoroalkyl, —CN, —OH, —OR²⁰, —N(R²¹)₂, or —NR²¹C(═O)R²¹;
each R¹³ is independently hydrogen, halogen, unsubstituted or substituted C₁-C₆alkyl, —CN, —OH, —OR²⁰, —CO₂R²¹, —C(═O)N(R²¹)₂, —N(R²¹)₂, or —NR²¹C(═O)R²¹;
p is 0, 1, or 2.

63. The compound of claim 56, or a pharmaceutically acceptable salt thereof, wherein:
R⁶ and R⁷ are taken together with the intervening carbon atoms connecting R⁶ to R⁷ to form a ring B that is monocyclic C₃-C₈ aryl, wherein Ring B is unsubstituted or substituted with 1 or 2 R¹¹, or 0 or 1 R¹².

64. The compound of claim 63, or a pharmaceutically acceptable salt thereof, wherein: 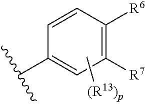
is

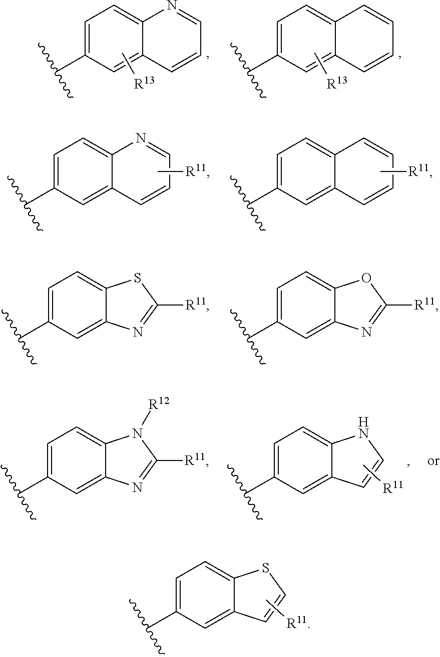

65. The compound of claim 56, or a pharmaceutically acceptable salt thereof, wherein:
R⁶ and R⁷ are taken together with the intervening carbon atoms connecting R⁶ to R⁷ to form a ring B that is monocyclic C₃-C₈cycloalkyl or monocyclic C₃-C₈heterocycloalkyl, wherein ring B is unsubstituted or substituted with 1 or 2 R¹¹, or 0 or 1 R¹².

66. The compound of claim 65, or a pharmaceutically acceptable salt thereof, wherein: 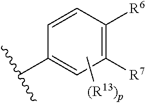
is

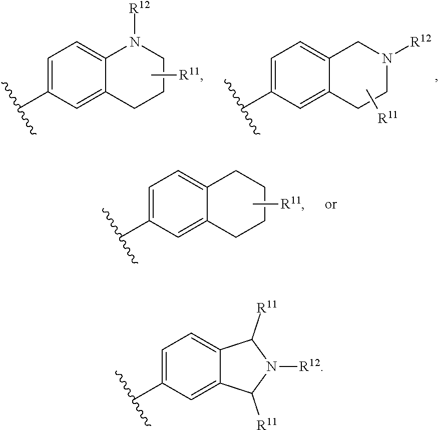

67. The compound of claim 56, wherein the compound has one of the following structures, or a pharmaceutically acceptable salt thereof:

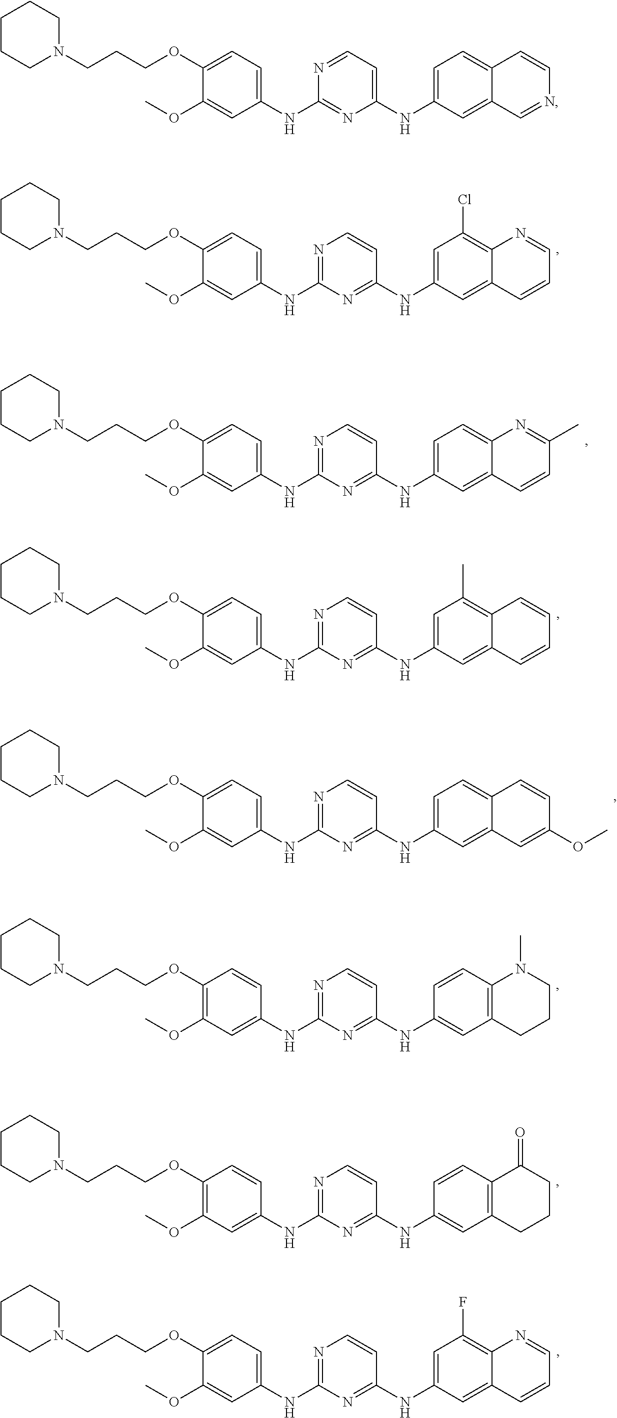

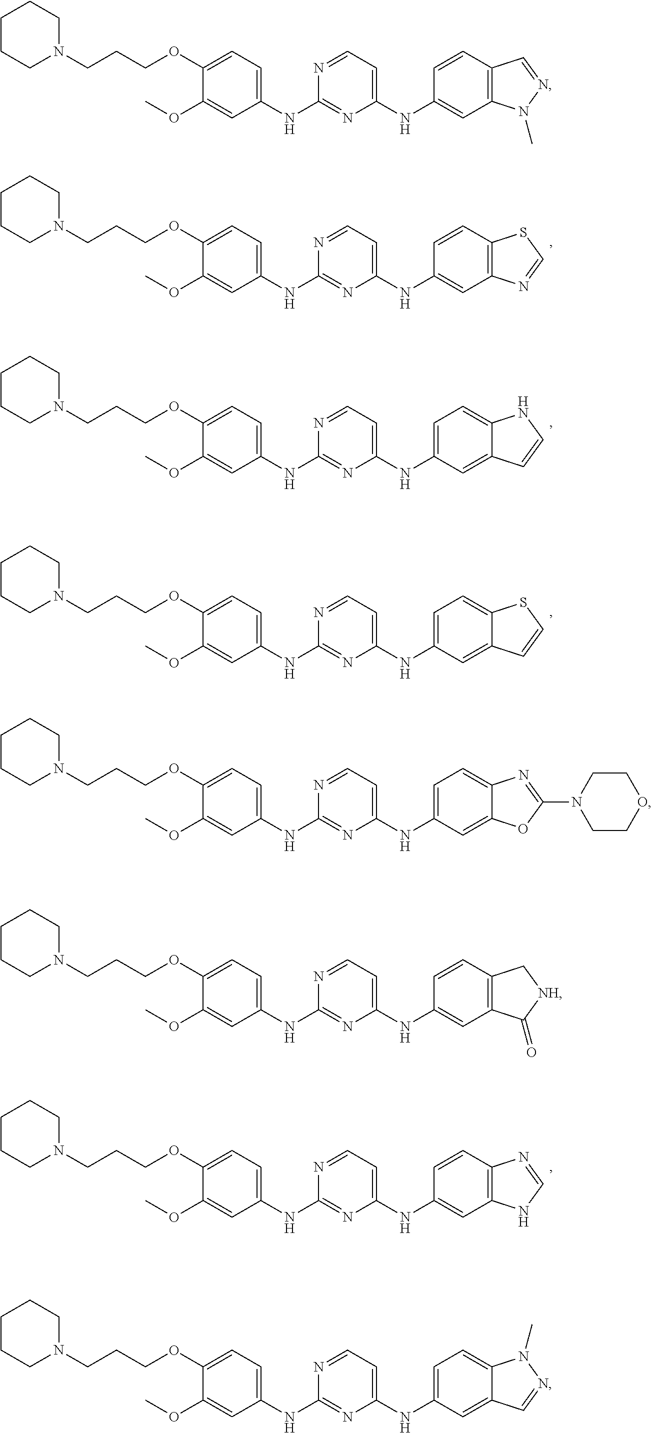

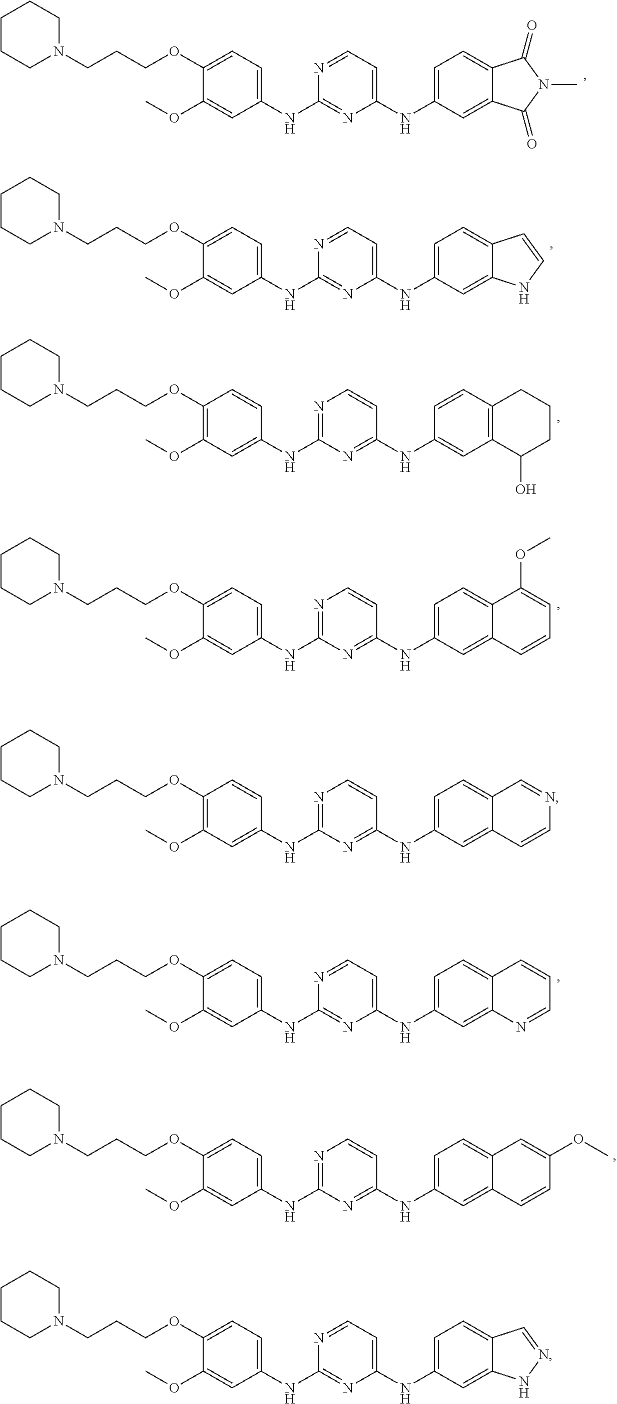

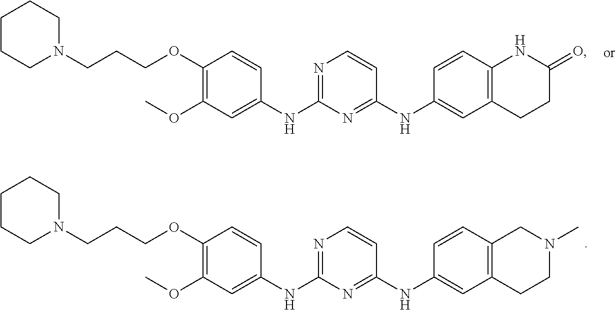 [SMILES: COc1cc(Nc2nccc(Nc3ccc4c(c3)CCC(=O)N4)n2)ccc1OCCCN1CCCCC1 ; COc1cc(Nc2nccc(Nc3ccc4c(c3)CCN(C)C4)n2)ccc1OCCCN1CCCCC1]

68. A compound of claim that has the structure of Formula (IV), or a pharmaceutically acceptable salt thereof: 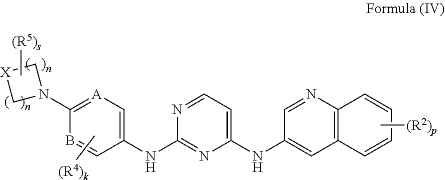
wherein:
each n is independently 1, 2, or 3;
each R⁵ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
s is 0, 1, 2, 3, or 4;
X is —C(R⁸)₂—, —N(R⁹)—, —O—, —S—, —S(═O)—, or —SO₂—;
each R⁸ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
or both R⁸ are taken together with the carbon to which they are attached to form a unsubstituted or substituted C₃-C₈cycloalkyl, unsubstituted or substituted C₃-C₈heterocycloalkyl;
R⁹ is hydrogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈heterocycloalkyl, —C(═O)R²¹, —CO₂R²¹, or —C(═O)N(R²¹)₂;
B is C(R³) or N;
R³ is hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²¹;
each R⁴ is independently hydrogen, halogen, unsubstituted or substituted C₁-C₆alkyl, or unsubstituted or substituted C₁-C₆alkoxy;
k is 0, 1, or 2;
A is C(R⁴) or N;
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OH, —OR²⁰, —C(═O)R²¹, —CO₂R²¹, —N(R²¹)₂, —SR²¹, —S(═O)R²⁰, or —SO₂R²¹;
p is 0, 1, 2, 3, or 4;
wherein
when 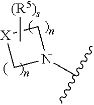
is 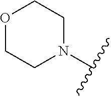 [SMILES: *N1CCOCC1]
and p is 0, at least one of R³ or R⁴ is —O-(unsubstituted C₁-C₆alkyl); or
when 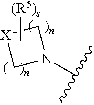
is 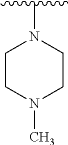 [SMILES: *N1CCN(C)CC1]
and p is 0, at least one of R³ or R⁴ is —O-(unsubstituted or substituted C₁-C₆alkyl);
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

69. The compound of claim 68, or a pharmaceutically acceptable salt thereof, wherein:
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, or —N(R²¹)₂.

70. The compound of claim 68, or a pharmaceutically acceptable salt thereof, wherein:
each R² is independently hydrogen or halogen.

71. The compound of claim 68, or a pharmaceutically acceptable salt thereof, wherein:
A is C(R⁴) and B is N;
or A is N and B is C(R³).

72. The compound of any one of claim 68, or a pharmaceutically acceptable salt thereof, wherein:
A is C-(unsubstituted or substituted C₁-C₆alkoxy) and B is N;
or A is N and B is C-(unsubstituted or substituted C₁-C₆alkoxy).

73. The compound of any one of claim 68, or a pharmaceutically acceptable salt thereof, wherein:
each n is independently 1 or 2;
each R⁵ is independently hydrogen, halogen, unsubstituted or substituted C₁-C₆alkyl, unsubstituted or substituted C₁-C₆fluoroalkyl, —CN, OH, —OR²⁰, or N(R²¹)₂; and
X is —C(R⁸)₂—, —N(R⁹)—, or —O—.

74. The compound of claim 68, or a pharmaceutically acceptable salt thereof, wherein:
X is —C(R⁸)₂—, —N(R⁹)—, or —O—;
each R⁸ is independently hydrogen, unsubstituted or substituted C₁-C₆alkyl, unsubstituted or substituted C₃-C₈cycloalkyl, unsubstituted or substituted C₃-C₈heterocycloalkyl, —OH, —N(R²¹)₂;
or both R⁸ are taken together with the carbon to which they are attached to form a unsubstituted or substituted C₃-C₈cycloalkyl, unsubstituted or substituted C₃-C₈heterocycloalkyl; and
R⁹ is hydrogen, unsubstituted or substituted C₁-C₆alkyl, unsubstituted or substituted C₃-C₈cycloalkyl, —CO₂R²¹, or —C(═O)N(R²¹)₂.

75. The compound of claim 68, or a pharmaceutically acceptable salt thereof, wherein:

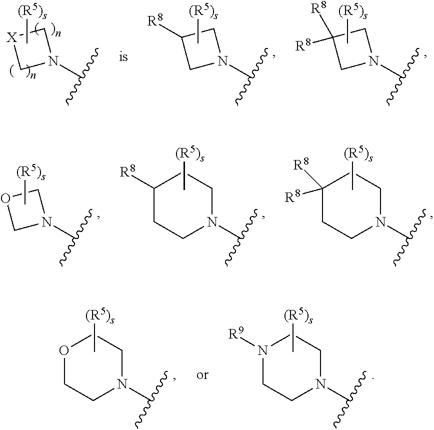

76. The compound of claim 68, or a pharmaceutically acceptable salt thereof, wherein: 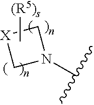
is

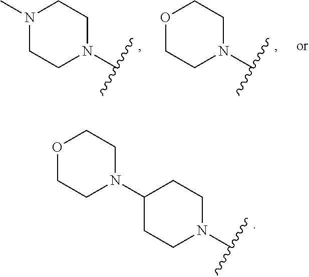

77. The compound of claim 68, wherein the compound has one of the following structures, or a pharmaceutically acceptable salt thereof:

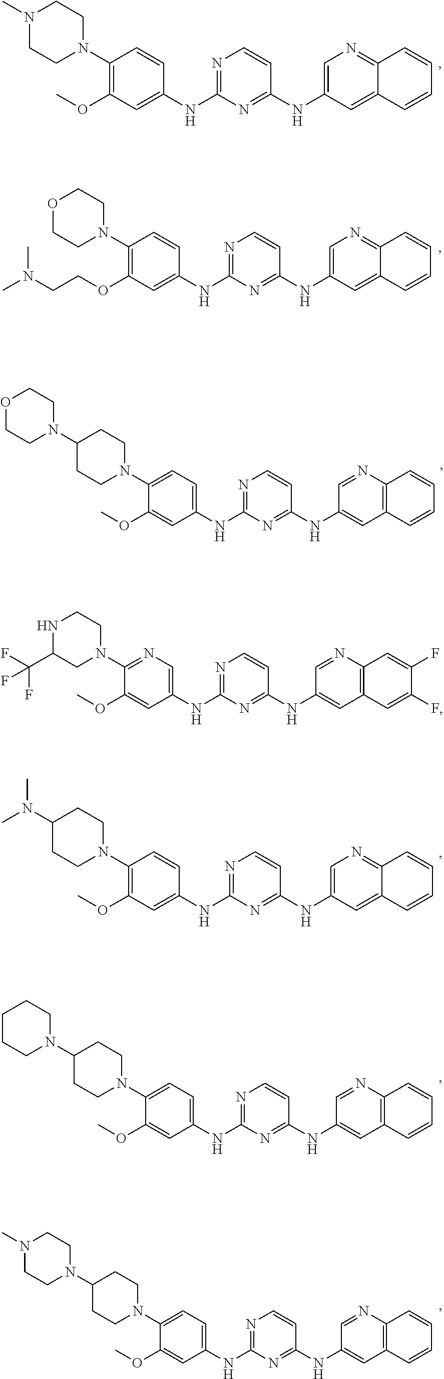

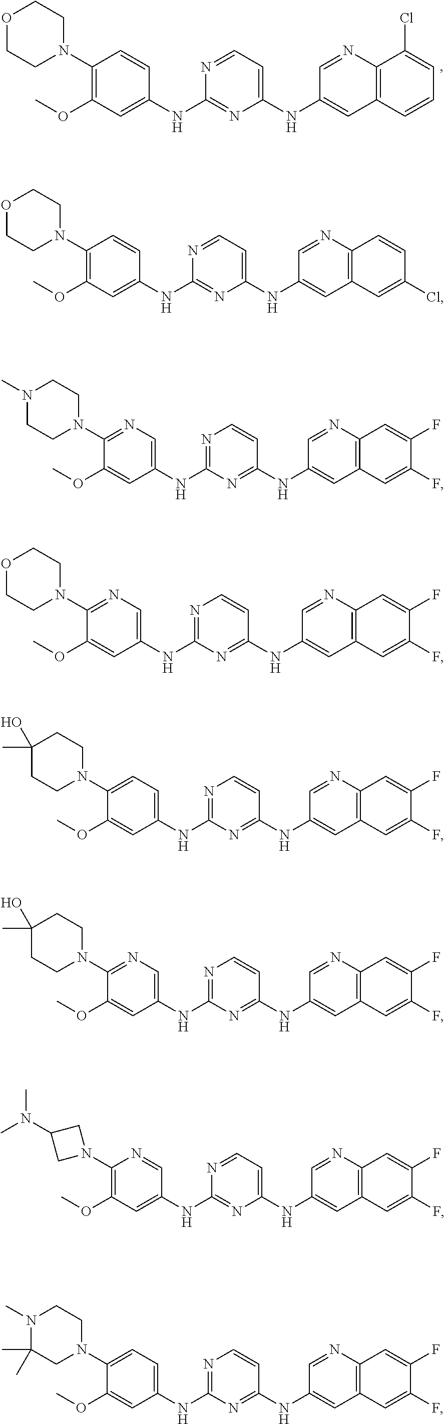

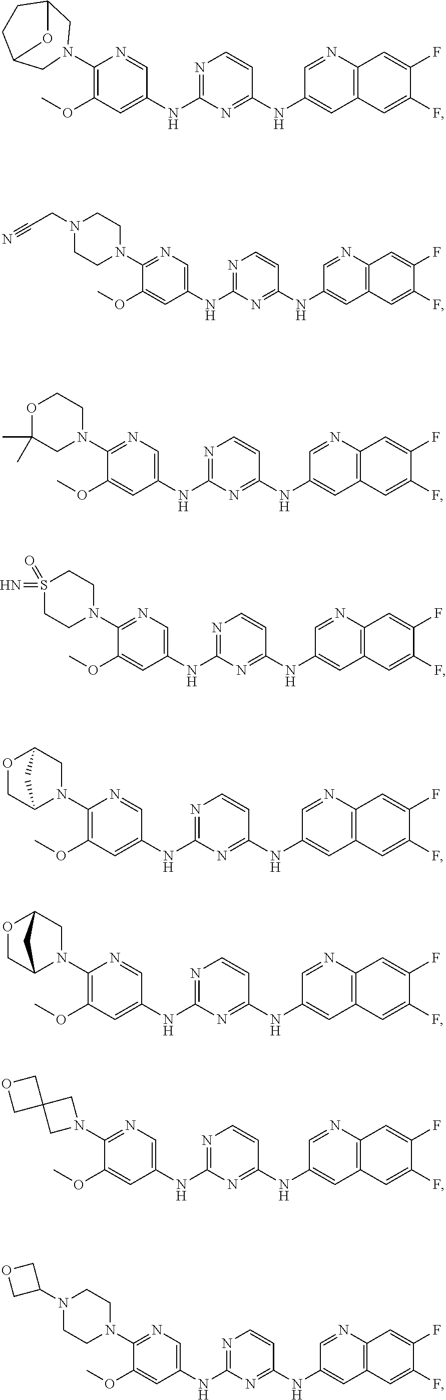

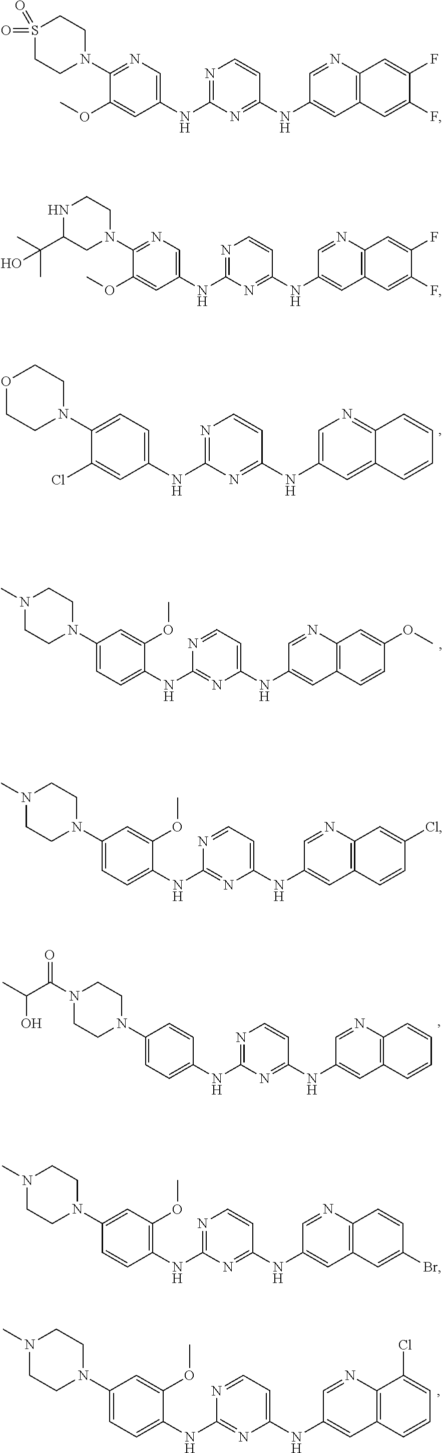

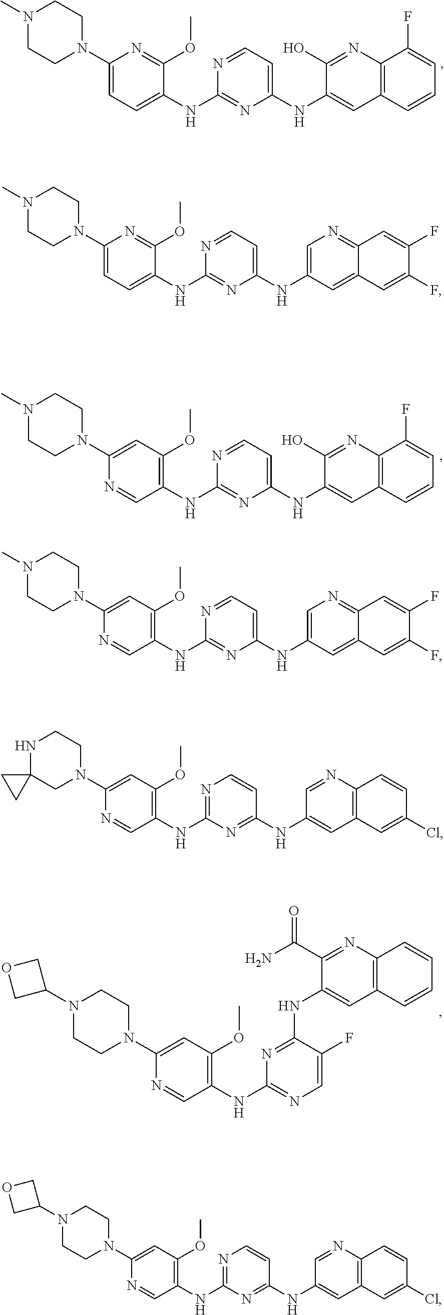

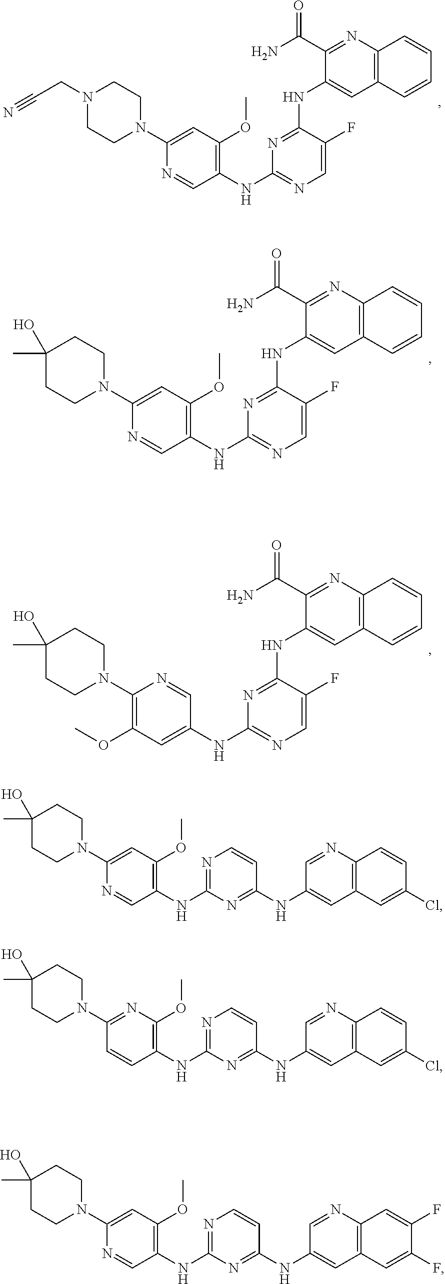 [SMILES: COc1cc(N2CCN(CC#N)CC2)ncc1Nc1ncc(F)c(Nc2cc3ccccc3nc2C(N)=O)n1 ; COc1cc(N2CCC(C)(O)CC2)ncc1Nc1ncc(F)c(Nc2cc3ccccc3nc2C(N)=O)n1 ; COc1cc(Nc2ncc(F)c(Nc3cc4ccccc4nc3C(N)=O)n2)cnc1N1CCC(C)(O)CC1 ; COc1cc(N2CCC(C)(O)CC2)ncc1Nc1nccc(Nc2cnc3ccc(Cl)cc3c2)n1 ; COc1nc(N2CCC(C)(O)CC2)ccc1Nc1nccc(Nc2cnc3ccc(Cl)cc3c2)n1 ; COc1cc(N2CCC(C)(O)CC2)ncc1Nc1nccc(Nc2cnc3cc(F)c(F)cc3c2)n1]

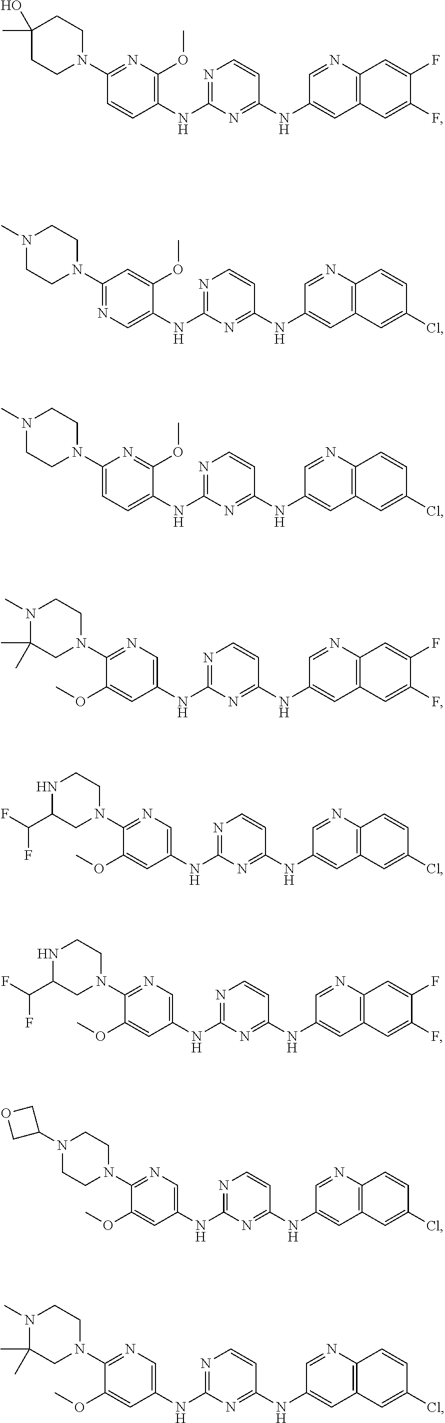

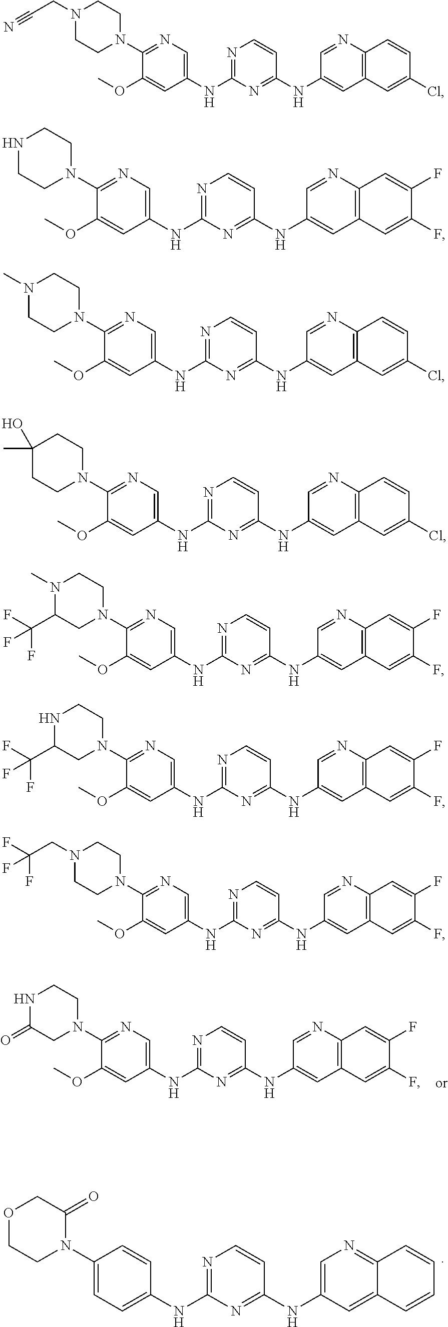

78. A compound of that has the structure of Formula (V), or a pharmaceutically acceptable salt thereof: 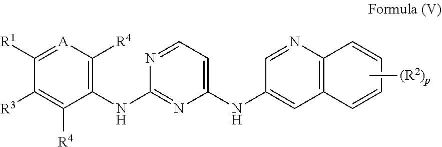
wherein:
R¹ and R³ are taken together with the intervening carbon atoms connecting R¹ to R³ to form a ring A that is 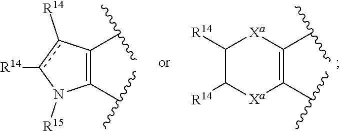
wherein [symbol] indicates a single or double bond;
each R¹⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, substituted or unsubstituted C₁-C₆alkyl-N(R²¹)₂, oxo, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
R¹⁵ is -substituted or unsubstituted C₁-C₆alkyl, -substituted or unsubstituted C₁-C₆alkyl-N(R²¹)₂, —C(═O)-substituted or unsubstituted C₁-C₆alkyl-N(R²¹)₂, -substituted or unsubstituted C₁-C₆alkyl-carbocycle, or heterocycle;
each X^{a} is independently —C(R¹⁶)₂—, —N(R¹⁷)—, —O—, or —S—;
each R¹⁶ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
each R¹⁷ is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —C(═O)R²¹, —CO₂R²¹, or —C(═O)N(R²¹)₂;
each R⁴ is independently hydrogen, halogen, unsubstituted or substituted C₁-C₆alkyl, unsubstituted or substituted C₁-C₆fluoroalkyl, unsubstituted or substituted C₁-C₆heteroalkyl, unsubstituted or substituted C₃-C₈cycloalkyl, unsubstituted or substituted C₃-C₈hetercycloalkyl, —CN, —OH, —OR²⁰, —C(═O)R²¹, —CO₂R²¹, —C(═O)N(R²¹)₂, —N(R²¹)₂, —NR²¹C(═O)R²¹, —NR²¹CO₂R²¹, —SR²¹, —S(═O)R²⁰, —SO₂R²¹, or —SO₂N(R²¹)₂;
A is C(R⁴) or N;
each R² is independently hydrogen, halogen, unsubstituted or substituted C₁-C₆alkyl, unsubstituted or substituted C₁-C₆fluoroalkyl, unsubstituted or substituted C₁-C₆heteroalkyl, —CN, —OH, —OR²⁰, —C(═O)R²¹, —CO₂R²¹, —N(R²¹)₂, —SR²¹, —S(═O)R²⁰, or —SO₂R²¹;
p is 0, 1, 2, 3, or 4;
each R²⁰ is independently selected from unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
each R²¹ is independently selected from hydrogen, unsubstituted or substituted C₁-C₆alkyl, unsubstituted or substituted C₁-C₆heteroalkyl, unsubstituted or substituted C₃-C₁₀cycloalkyl, substituted or unsubstituted C₂-C₁₀heterocycloalkyl, substituted or unsubstituted aryl, and substituted or unsubstituted heteroaryl;
or two R²¹ on the same N atom are taken together with the N atom to which they are attached to form an unsubstituted or substituted N-containing heterocycle.

79. The compound of claim 78, or a pharmaceutically acceptable salt thereof, wherein:
each R² is independently hydrogen, halogen, unsubstituted or substituted C₁-C₆alkyl, or unsubstituted or substituted C₁-C₆fluoroalkyl.

80. The compound of claim 78, or a pharmaceutically acceptable salt thereof, wherein:
each R¹⁴ is independently hydrogen, halogen, unsubstituted or substituted C₁-C₆alkyl, unsubstituted or substituted C₁-C₆fluoroalkyl, -(unsubstituted or substituted C₁-C₆alkyl)-N(R²¹)₂, oxo, or —CN;
R¹⁵ is unsubstituted or substituted C₁-C₆alkyl, -(unsubstituted or substituted C₁-C₆alkyl)-N(R²¹)₂, —C(═O)— unsubstituted or substituted C₁-C₆alkyl)-N(R²¹)₂, -(unsubstituted or substituted C₁-C₆alkyl)-carbocycle, or heterocycle;
each X^{a} is independently —N(R¹⁷)— or —O—.

81. The compound of claim 78, or a pharmaceutically acceptable salt thereof, wherein:
R¹ and R³ are taken together with the intervening carbon atoms connecting R¹ to R³ to form a ring A that is 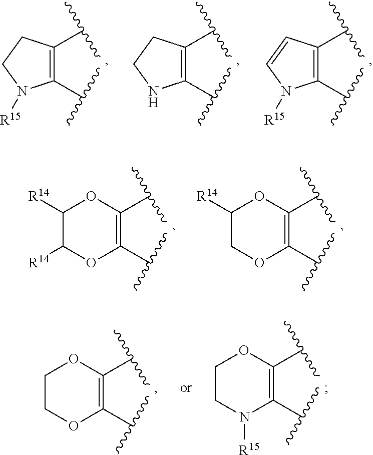
each R¹⁴ is independently halogen, C₁-C₆alkyl, C₁-C₆fluoroalkyl, —C₁-C₆alkyl-N(R²¹)₂, or oxo; and
R¹⁵ is -(unsubstituted or substituted C₁-C₆alkyl)-N(R²¹)₂, —C(═O)-(unsubstituted or substituted C₁-C₆alkyl)-N(R²¹)₂, -(unsubstituted or substituted C₁-C₆alkyl)-(unsubstituted or substituted C₃-C₈carbocycle), or unsubstituted or substituted C₃-C₈heterocycle.

82. The compound of claim 78, or a pharmaceutically acceptable salt thereof, wherein:
each R¹⁴ is independently hydrogen or —CH₂—N(CH₃)₂; and
R¹⁵ is —CH₂CH₂—N(CH₃)₂, —C(═O)—CH₂—N(CH₃)₂, —CH₂—(C₃-C₈ monocyclic carbocycle), or C₃-C₈heterocycle.

83. The compound of claim 78, wherein the compound has one of the following structures, or a pharmaceutically acceptable salt thereof:

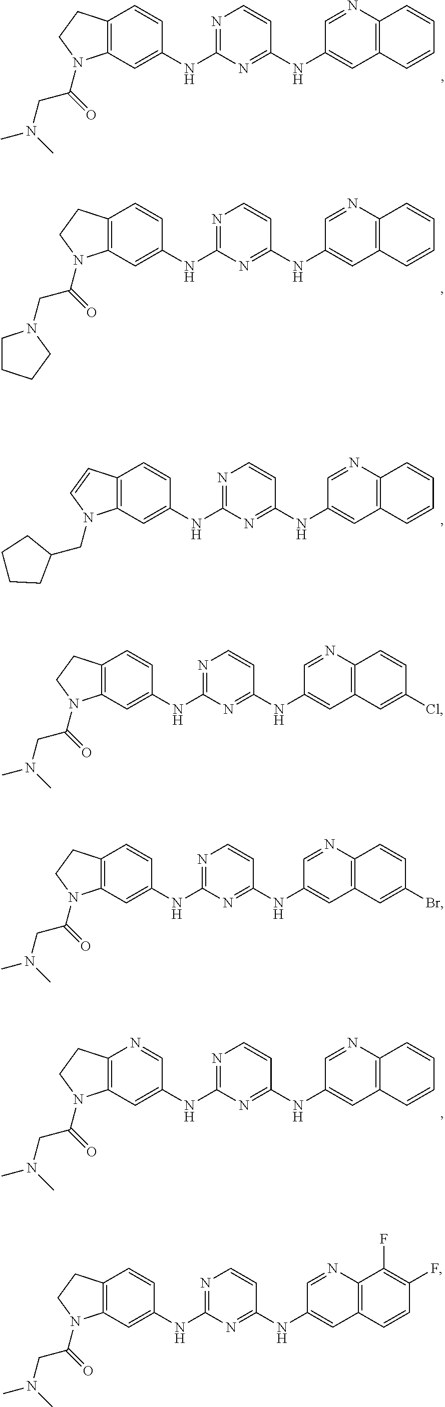

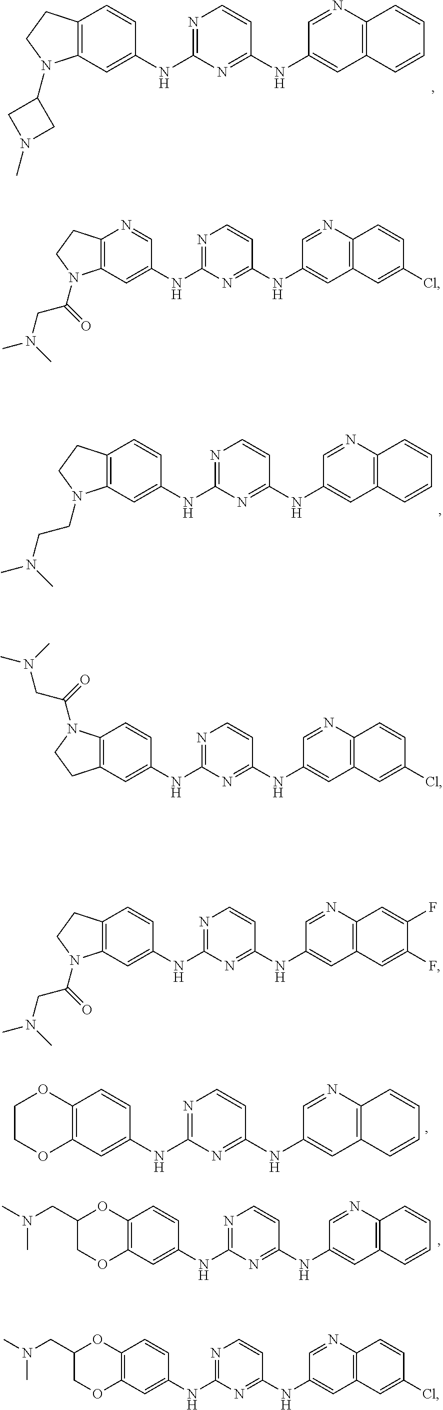

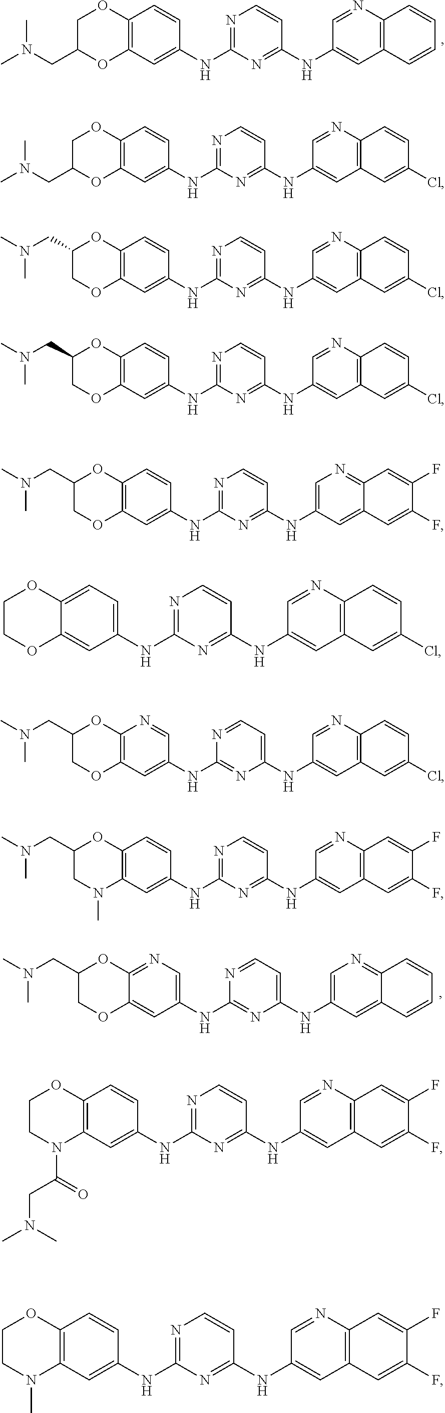

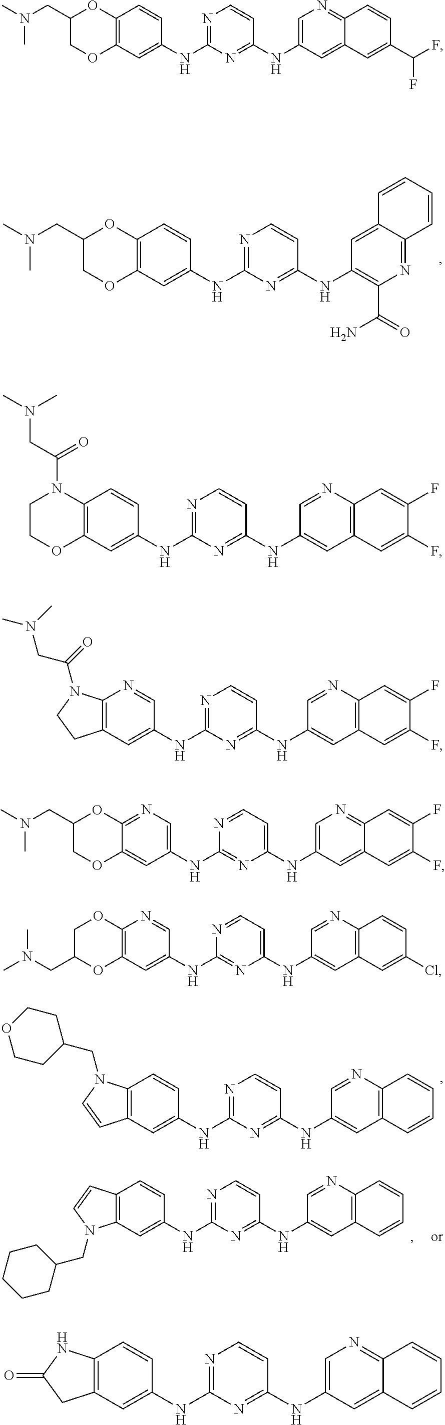

84. A pharmaceutical composition comprising a compound of any one of claims 1, 22, 35, 42, 49, 56, 68 and 78, or a pharmaceutically acceptable salt thereof, and at least one pharmaceutically acceptable excipient.

85. The pharmaceutical composition of claim 84, wherein the pharmaceutical composition is formulated for administration to a mammal by intravenous administration, subcutaneous administration, oral administration, inhalation, nasal administration, dermal administration, or ophthalmic administration.

86. The pharmaceutical composition of claim 85, wherein the pharmaceutical composition is in the form of a tablet, a pill, a capsule, a liquid, a suspension, a gel, a dispersion, a solution, an emulsion, an ointment, or a lotion.

87. Use of a compound of any one of claims 1, 22, 35, 42, 49, 56, 68 and 78, or a pharmaceutically acceptable salt thereof, in the treatment of a disease or condition in a mammal that would benefit from the modulation of insulin-like growth factor-1 receptor (IGF-1R).
